(19) United States
(12) Patent Application Publication
(10) Pub. No.: US 2025/0195483 A1
(43) Pub. Date: Jun. 19, 2025

(54) SPIRO PIPERIDINE DERIVATIVES AS INHIBITORS OF APOL1 AND METHODS OF USING SAME

(71) Applicant: Vertex Pharmaceuticals Incorporated, Boston, MA (US)

(72) Inventors: Timothy J. SENTER, Arlington, MA (US); Ming CHEN, Medford, MA (US); Leslie A. DAKIN, Framingham, MA (US); Zachary GALE-DAY, Brookline, MA (US); Sheila NAMIREMBE, Newton, MA (US); Jessica H. OLSEN, Jamaica Plain, MA (US); Akira J. SHIMIZU, Framingham, MA (US); Haoxuan WANG, Somerville, MA (US); Tiansheng WANG, Concord, MA (US)

(21) Appl. No.: 18/836,681

(22) PCT Filed: Feb. 8, 2023
(86) PCT No.: PCT/US2023/012583

§ 371 Date: Aug. 7, 2024

Related U.S. Application Data
(60) Provisional application No. 63/307,926, filed on Feb. 8, 2022.

(51) Int. Cl. A61K 31/438; A61K 31/444; A61K 31/506; C07D 401/06; C07D 401/14; C07D 471/10; C07D 491/107; C07D 495/10

(52) U.S. Cl. CPC A61K 31/438; A61K 31/444; A61K 31/506; C07D 401/06; C07D 401/14; C07D 471/10; C07D 491/107; C07D 495/10

(57) ABSTRACT

The disclosure provides at least one compound, tautomer, deuterated derivative, or pharmaceutically acceptable salt chosen from compounds of Formula I, tautomers thereof, deuterated derivatives of those compounds or tautomers, and pharmaceutically acceptable salts of any of the foregoing, compositions comprising the same, and methods of using the same, including uses in treating APOL1-mediated diseases, including pancreatic cancer, focal segmental glomerulosclerosis (FSGS), and/or non-diabetic kidney disease (NDKD).

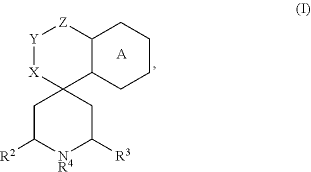

This application claims the benefit of priority of U.S. Provisional Application No. 63/307,926, filed Feb. 8, 2022, the entire disclosure of which is incorporated herein by reference.

This disclosure provides compounds that may inhibit apolipoprotein L1 (APOL1) and methods of using those compounds to treat APOL1-mediated diseases, such as, e.g., pancreatic cancer, focal segmental glomerulosclerosis (FSGS), and/or non-diabetic kidney disease (NDKD). In some embodiments, the FSGS and/or NDKD is associated with at least one of the 2 common APOL1 genetic variants (G1: S342G:I384M and G2: N388del:Y389del). In some embodiments, the pancreatic cancer is associated with elevated levels of APOL1 (such as, e.g., elevated levels of APOL1 in pancreatic cancer tissues).

FSGS is a rare kidney disease with an estimated global incidence of 0.2 to 1.1/100,000/year. FSGS is a disease of the podocyte (glomerular visceral epithelial cells) responsible for proteinuria and progressive decline in kidney function. NDKD is a kidney disease involving damage to the podocyte or glomerular vascular bed that is not attributable to diabetes. NDKD is a disease characterized by hypertension and progressive decline in kidney function. Human genetics support a causal role for the G1 and G2 APOL1 variants in inducing kidney disease. Individuals with 2 APOL1 alleles are at increased risk of developing end-stage kidney disease (ESKD), including primary (idiopathic) FSGS, human immunodeficiency virus (HIV) associated FSGS, NDKD, arterionephrosclerosis, lupus nephritis, microalbuminuria, and chronic kidney disease. See, P. Dummer et al., Semin Nephrol. 35(3): 222-236 (2015).

FSGS and NDKD can be divided into different subgroups based on the underlying etiology. One homogeneous subgroup of FSGS is characterized by the presence of independent common sequence variants in the apolipoprotein L1 (APOL1) gene termed G1 and G2, which are referred to as the “APOL1 risk alleles.” G1 encodes a correlated pair of non-synonymous amino acid changes (S342G and I384M), G2 encodes a 2 amino acid deletion (N388del:Y389del) near the C terminus of the protein, and G0 is the ancestral (low risk) allele. A distinct phenotype of NDKD is found in patients with APOL1 genetic risk variants as well. In both APOL1-mediated FSGS and NDKD, higher levels of proteinuria and a more accelerated loss of kidney function occur in patients with two risk alleles compared to patients with the same disease who have no or just 1 APOL1 genetic risk variant. Alternatively in AMKD, higher levels of proteinuria and accelerated loss of kidney function can also occur in patients with one risk allele. See, G. Vajgel et al., J. Rheumatol., November 2019, jrheum.190684.

APOL1 is a 44 kDa protein that is only expressed in humans, gorillas, and baboons. The APOL1 gene is expressed in multiple organs in humans, including the liver and kidney. APOL1 is produced mainly by the liver and contains a signal peptide that allows for secretion into the bloodstream, where it circulates bound to a subset of high-density lipoproteins. APOL1 is responsible for protection against the invasive parasite, Trypanosoma brucei brucei (T. b. brucei). APOL1 is endocytosed by T. b. brucei and transported to lysosomes, where it inserts into the lysosomal membrane and forms pores that lead to parasite swelling and death.

While the ability to lyse T. b. brucei is shared by all 3 APOL1 variants (G0, G1, and G2), APOL1 G1 and G2 variants confer additional protection against parasite species that have evolved a serum resistant associated-protein (SRA) which inhibits APOL1 G0; APOL1 G1 and G2 variants confer additional protection against trypanosoma species that cause sleeping sickness. G1 and G2 variants evade inhibition by SRA; G1 confers additional protection against T. b. gambiense (which causes West African sleeping sickness) while G2 confers additional protection against T. b. rhodesiense (which causes East African sleeping sickness).

In the kidney, APOL1 is expressed in podocytes, endothelial cells (including glomerular endothelial cells), and some tubular cells. Podocyte-specific expression of APOL1 G1 or G2 (but not G0) in transgenic mice induces structural and functional changes, including albuminuria, decreased kidney function, podocyte abnormalities, and glomerulosclerosis. Consistent with these data, G1 and G2 variants of APOL1 play a causative role in inducing FSGS and accelerating its progression in humans. Individuals with APOL1 risk alleles (i.e., homozygous or compound heterozygous for the APOL1 G1 or APOL1 G2 alleles) have increased risk of developing FSGS and they are at risk for rapid decline in kidney function if they develop FSGS. Thus, inhibition of APOL1 could have a positive impact in individuals who harbor APOL1 risk alleles.

Although normal plasma concentrations of APOL1 are relatively high and can vary at least 20-fold in humans, circulating APOL1 is not causally associated with kidney disease. However, APOL1 in the kidney is thought to be responsible for the development of kidney diseases, including FSGS and NDKD. Under certain circumstances, APOL1 protein synthesis can be increased by approximately 200-fold by pro-inflammatory cytokines such as interferons or tumor necrosis factor-a. In addition, several studies have shown that APOL1 protein can form pH-gated Na⁺/K⁺ pores in the cell membrane, resulting in a net efflux of intracellular K⁺, ultimately resulting in activation of local and systemic inflammatory responses, cell swelling, and death.

The risk of ESKD is substantially higher in people of recent sub-Saharan African ancestry as compared to those of European ancestry. In the United States, ESKD is responsible for nearly as many lost years of life in women as from breast cancer and more lost years of life in men than from colorectal cancer.

FSGS and NDKD are caused by damage to podocytes, which are part of the glomerular filtration barrier, resulting in proteinuria. Patients with proteinuria are at a higher risk of developing end-stage kidney disease (ESKD) and developing proteinuria-related complications, such as infections or thromboembolic events. There is no standardized treatment regimen nor approved drugs for FSGS or NDKD. Currently, FSGS and NDKD are managed with symptomatic treatment (including blood pressure control using blockers of the renin angiotensin system), and patients with FSGS and heavy proteinuria may be offered high dose steroids. Current therapeutic options for NDKD are anchored on blood pressure control and blockade of the renin angiotensin system.

Corticosteroids, alone or in combination with other immunosuppressants, induce remission in a minority of patients (e.g., remission of proteinuria in a minority of patients) and are associated with numerous side effects. However, remission is frequently indurable even in patients initially responsive to corticosteroid and/or immunosuppressant treatment. As a result, patients, in particular individuals of recent sub-Saharan African ancestry with 2 APOL1 risk alleles, experience rapid disease progression leading to end-stage renal disease (ESRD). Thus, there is an unmet medical need for treatment for FSGS and NDKD. Illustratively, in view of evidence that APOL1 plays a causative role in inducing and accelerating the progression of kidney disease, inhibition of APOL1 should have a positive impact on patients with APOL1 mediated kidney disease, particularly those who carry two APOL1 risk alleles (i.e., are homozygous or compound heterozygous for the G1 or G2 alleles).

Additionally, APOL1 is an aberrantly expressed gene in multiple cancers (Lin et al., Cell Death and Disease (2021), 12:760). Recently, APOL1 was found to be abnormally elevated in human pancreatic cancer tissues compared with adjacent tissues and was associated with poor prognosis in pancreatic cancer patients. In in vivo and in vitro experiments, knockdown of APOL1 significantly inhibited cancer cell proliferation and promoted the apoptosis of pancreatic cancer cells.

One aspect of the disclosure provides at least one compound, tautomer, deuterated derivative, or pharmaceutically acceptable salt chosen from compounds of Formula I, tautomers of Formula I, deuterated derivatives of those compounds or tautomers, and pharmaceutically acceptable salts of any of the foregoing, which can be employed in the treatment of diseases mediated by APOL1, such as FSGS and NDKD. For example, in some embodiments, the at least one compound is a compound represented by Formula I:

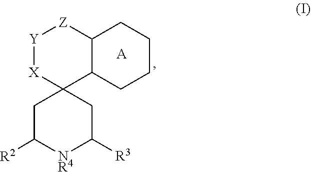

a tautomer thereof, a deuterated derivative of that compound or tautomer, or a pharmaceutically acceptable salt of any of the foregoing, wherein:
Ring A is chosen from 6-membered aryl and 6-membered heteroaryl groups, wherein the 6-membered aryl or 6-membered heteroaryl group is optionally substituted by 1, 2, 3, or 4 R¹ groups;
X is chosen from —CR^{1a}R^{1b}—, —C(O)—, —S—, —S(O)₂—, —NR^{1c}—, and —O—;
Y is chosen from —CR^{1a}R^{1b}—, —C(O)—, —S(O)₂—, —NR^{1c}—, and —O—;
Z is chosen from a bond, —CR^{1a}R^{1b}—, —NR^{1c}—, —C(O)—, —S(O)₂—, and —O—, wherein:
   at least one of X and Y is chosen from —CR^{1a}R^{1b}— and —C(O)—;
R^{1a}, R^{1b}, and R^{1c}, for each occurrence, are each independently chosen from hydrogen and R¹ groups;
R¹, for each occurrence, is independently chosen from halogen, —OH, cyano, phenyl, C₁-C₆ alkyl, C₁-C₆ alkenyl, C₁-C₆ alkoxy, C₃-C₆ carbocyclyl, 4- to 6-membered heterocyclyl, —C(═O)R^{c}, —C(═O)OR^{c}, —C(═O)N(R^{c})₂, and —OS(═O)₂R^{c} groups, wherein:
   R^{c}, for each occurrence, is independently chosen from hydrogen, C₁-C₄ alkyl, and C₁-C₄ haloalkyl groups;
   the 4- to 6-membered heterocyclyl of R¹ comprises 1 to 2 heteroatoms independently chosen from nitrogen and oxygen and is optionally substituted with a group selected from oxo and —OH;
   the C₁-C₆ alkyl and C₁-C₆ alkenyl of R¹ are each optionally substituted with 1 to 4 groups independently chosen from halogen, cyano, —OH, —NH₂, —NH(C₁-C₄ alkyl), —N(C₁-C₄ alkyl)₂, C₁-C₄ alkoxy, 3- to 5-membered heterocyclyl (optionally substituted with R¹), 3- to 5-membered heteroaryl groups, and phenyl;
   the C₁-C₆ alkoxy of R¹ is optionally substituted with 1 to 3 groups independently chosen from —OH, cyano, and halogen groups;
   the C₃-C₆ carbocyclyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups; and
   the phenyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups;
R² and R³ are each independently chosen from hydrogen and C₁-C₄ alkyl groups;
R⁴ is chosen from C₁-C₆ alkyl, —C(═O)O(C₁-C₄ alkyl), and

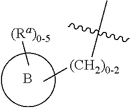

groups, wherein:
the C₁-C₆ alkyl of R⁴ is optionally substituted with 1 to 5 groups independently chosen from halogen, cyano, —OH, —NH₂, —NH(C₁-C₄ alkyl), —N(C₁-C₄ alkyl)₂, —C(═O)NH₂, —C(═O)(C₁-C₄ alkyl), —C(═O)OH, —C(═O)O(C₁-C₄ alkyl), —C(═O)NH(C₁-C₄ alkyl), —C(═O)N(C₁-C₄ alkyl)₂, C₁-C₄ alkoxy, C₃-C₆ carbocyclyl, C₆ aryl, —O—(C₆ aryl), 5- to 10-membered heterocyclyl, and 5- to 10-membered heteroaryl groups, wherein:
   the C₆ aryl and —O—(C₆ aryl) groups are each optionally substituted with 1 to 3 groups independently chosen from halogen and C₁-C₄ haloalkyl groups;
Ring B is chosen from C₃-C₁₂ carbocyclyl, 3- to 12-membered heterocyclyl, C₆ and C₁₀ aryl, and 5- to 10-membered heteroaryl groups, wherein Ring B is optionally substituted with 1, 2, 3, 4, or 5 R^{a} groups; wherein:
   R^{a}, for each occurrence, is independently chosen from halogen, cyano, oxo, C₁-C₈ alkyl, C₁-C₆ haloalkyl, C₂-C₅ alkenyl, C₁-C₆ haloalkenyl, C₁-C₆ alkoxy, C₁-C₆ haloalkoxy, C₃-C₁₂ carbocyclyl, C₆ and C₁₀ aryl, 3- to 12-membered heterocyclyl, 5- to 10-membered heteroaryl, —C(═O)NR^{h}Rⁱ, —C(═O)OR^{k},
—C(═O)(C₁-C₄ alkylene)OR^{k}, —C(═O)R^{k}, —C(═O)(C₁-C₄ alkylene)S(═O)ₚR^{k}, —C(═O)(C₁-C₄ alkylene)S(═O)ₚN^{h}Rⁱ, —C(═O)(C₁-C₄ alkylene)NRⁱS(═O)ₚR^{k}, —C(═O)(C₁-C₄ alkylene)NR^{h}C(═O)R^{k}, —C(═O)C(═O)R^{k}, —NR^{h}Rⁱ, —NH(CH₂)_{q}CHR^{h}Rⁱ, —NH(CH₂)_{q}NR^{h}Rⁱ, —NR^{h}C(═O)R^{k}, —NR^{h}C(═O)OR^{k}, —NR^{h}C(═O)(C₁-C₄ alkylene)OR^{k}, —NR^{h}C(═O)O(C₁-C₄ alkylene)R^{k}, —NR^{h}C(═O)NRⁱR^{j}, —NR^{h}C(═O)(C₁-C₄ alkylene)NRⁱS(═O)ₚR^{k}, —NR^{h}S(═O)ₚR^{k}, —NR^{h}C(═O)(C₁-C₄ alkylene)S(═O)ₚR^{k}, —NR^{h}S(O)ₚ(C₁-C₄ alkylene)C(═O)OR^{k}, —
NR^{h}C(═O)[O(CH₂)_{q}]ᵣOC(═O)NR^{h}Rⁱ(CH₂)_{q}[O(CH₂)_{q}]ᵣ(C₁-C₆ alkyl) (optionally substituted by 1 to 3 R^{m} groups), —NR^{h}C(═O)(C₁-C₆ alkylene)[O(CH₂)_{q}]ᵣOC(═O)NR^{h}Rⁱ(CH₂)_{q}[O(CH₂)_{q}]ᵣ(C₁-C₆ alkyl) (optionally substituted by 1 to 3 R^{m} groups), —OR^{k}, —OC(═O)R^{k}, —OC(═O)OR^{k}, —OC(═O)NR^{h}Rⁱ, —[O(CH₂)_{q}]ᵣO(C₁-C₆ alkyl), —S(═O)ₚR^{k}, and —S(═O)ₚNR^{h}Rⁱ groups, wherein:
   the C₁-C₄ alkylene in each of —C(═O)(C₁-C₄ alkylene)S(═O)ₚR^{k}, —C(═O)(C₁-C₄ alkylene)OR^{k}, —C(═O)(C₁-C₄ alkylene)S(═O)ₚNR^{h}Rⁱ, —C(═O)(C₁-C₄ alkylene)NRⁱS(═O)ₚR^{k}, —C(═O)(C₁-C₄ alkylene)NR^{h}C(═O)R^{k}, —NR^{h}C(═O)O(C₁-C₄ alkylene)R^{k}, —NR^{h}C(═O)(C₁-C₄ alkylene)OR^{k}, —NR^{h}S(═O)ₚ(C₁-C₄ alkylene)C(═O)OR^{k}, and —NR^{h}C(═O)(C₁-C₄ alkylene)NRⁱS(═O)ₚR^{k} of R^{a} are optionally substituted with 1 to 3 —OH groups;
   the C₁-C₈ alkyl, the C₁-C₆ haloalkyl, the C₁-C₆ alkoxy, and the C₂-C₅ alkenyl of R^{a} are each optionally substituted with 1 to 3 groups independently chosen from cyano, —C(═O)R^{k}, —C(═O)OR^{k}, —C(═O)NR^{h}Rⁱ, =NOR^{k}, —NR^{h}Rⁱ, —NR^{h}C(═O)R^{k}, —NR^{h}C(═O)OR^{k}, —NR^{h}C(═O)NRⁱR^{j}, —NR^{h}S(═O)ₚR^{k}, —OR^{k}, —[O(CH₂)_{q}]ᵣOH, —OC(═O)R^{k}, —OC(═O)OR^{k}, —OC(═O)NR^{h}Rⁱ, —SR^{k}, —S(═O)R^{k}, —S(═O)ₚNR^{h}Rⁱ, —[O(CH₂)_{q}]ᵣO(C₁-C₄ alkyl), —O—(C₆ aryl or 5- to 8-membered heteroaryl) (optionally substituted with 1 to 3 R^{m} groups), C₃-C₆ carbocyclyl (optionally substituted with 1 to 3 R^{m} groups), C₆ to C₁₀ aryl (optionally substituted with 1 to 3 R^{m} groups), 4- to 10-membered heterocyclyl (optionally substituted with 1 to 3 R^{m} groups), and 5- to 10-membered heteroaryl (optionally substituted with 1 to 3 R^{m} groups) groups;
   the C₃-C₁₂ carbocyclyl, the 3- to 12-membered heterocyclyl, the C₆ and C₁₀ aryl, and the 5- to 10-membered heteroaryl of R^{a} are each optionally substituted with 1 to 3 groups independently chosen from halogen, oxo, cyano, C₁-C₆ alkyl (optionally substituted with 1 to 3 R^{m} groups), —C(═O)R^{k},
—C(═O)OR^{k}, —NR^{h}Rⁱ, —OR^{k}, —S(═O)ₚR^{k}, —S(═O)ₚNR^{h}Rⁱ, and 5- to 10-membered heterocyclyl groups, wherein:
   R^{h}, Rⁱ,and R^{j}, for each occurrence, are each independently chosen from hydrogen, C₁-C₆ alkyl, C₆-C₁₀ aryl, C₃-C₈ carbocyclyl (optionally substituted with 1 to 3 R^{m} groups), 5- to 10-membered heteroaryl (optionally substituted with 1 to 3 R^{m} groups), and 5- to 10-membered heterocyclyl (optionally substituted with 1 to 3 R^{m} groups) groups, wherein:
      the C₁-C₆ alkyl of any one of R^{h}, Rⁱ, and R^{j} is optionally substituted with 1 to 3 groups independently chosen from halogen, cyano, —OH, C₁-C₄ alkoxy, —C(═O)NH(C₁-C₄ alkyl), C₃-C₆ carbocyclyl (optionally substituted with 1 to 3 R^{m} groups), 5- to 10-membered heteroaryl (optionally substituted with 1 to 3 R^{m}groups), and 5- to 10-membered heterocyclyl (optionally substituted with 1 to 3 R^{m} groups) groups;
   R^{k}, for each occurrence, is independently chosen from hydrogen, C₁-C₆ alkyl, benzyl, C₆ aryl, C₃-C₆ carbocyclyl, 5-to 10-membered heterocyclyl, and 5- to 10-membered heteroaryl groups, wherein:
      the C₁-C₆ alkyl of any one of R^{k} is optionally substituted with 1 to 5 groups independently chosen from halogen, cyano, —NH₂, —OH, C₁-C₄ alkoxy, C₃-C₆ cycloalkyl (optionally substituted with 1 to 3 halogen groups), 5- to 10-membered heterocyclyl (optionally substituted with 1 to 3 —OH groups), and 5- to 10-membered heteroaryl (optionally substituted with 1 to 3 —OH groups) groups; and
      the C₃-C₆ carbocyclyl, benzyl, and C₆ aryl of any one of R^{k} are each optionally substituted with 1 to 3 groups independently chosen from halogen, cyano, oxo, —OH, —C(═O)NH₂, —C(═O)N(CH₃)₂, C₁-C₆ alkyl, C₁-C₆ haloalkyl, C₁-C₆ alkoxy, C₃-C₆ cycloalkyl (optionally substituted with 1 to 3 halogen groups), C₆ aryl (optionally substituted with 1 to 3 halogen groups), and 5- to 10-membered heteroaryl (optionally substituted with 1 to 3 halogen groups) groups, wherein:
      the C₁-C₄ alkyl is optionally substituted by 1 to 3 —OH groups; and
      the 5- to 10-membered heteroaryl and 5- to 10-membered heterocyclyl of any one of R^{k} are each optionally substituted with 1 to 3 groups independently chosen from halogen, cyano, —C(═O)CH₃, —NH₂, —OH, C₁-C₄ alkyl, C₁-C₄ haloalkyl, C₃-C₆ cycloalkyl, and C₁-C₄ alkoxy groups, wherein:
      the C₁-C₄ alkyl is optionally substituted by 1 to 3 —OH groups;
   R^{m}, for each occurrence, is independently chosen from halogen, cyano, oxo, —(CH₂)ₙC(═O)NH₂, —NH₂, —NH(C₁-C₄ alkyl), —N(C₁-C₄ alkyl)₂, C₁-C₆ alkyl, C₁-C₆ alkoxy, —C(═O)R^{k}, —S(═O)ₚR^{k}, —OR^{k}, C₃-C₆ cycloalkyl, and 5- to 10-membered heterocyclyl groups, wherein:
      the C₁-C₆ alkyl, the C₁-C₆ alkoxy, and the 5- to 10-membered heterocyclyl of any one of R^{m} is optionally substituted with 1 to 3 groups independently chosen from halogen, cyano, —OH, and C₁-C₄ alkoxy groups; and
n, for each occurrence, is an integer chosen from 0, 1, and 2;
p, for each occurrence, is an integer independently chosen from 1 and 2; and
q and r, for each occurrence, are each an integer independently chosen from 0, 1, 2, and 3.

In some embodiments, at least one compound, tautomer, deuterated derivative, or pharmaceutically acceptable salt of the disclosure is a compound represented by the structural Formulae IA, II, IIA, III, IV, IVA, IVB, IVC, V, VA, VB, VC, VI, VIA, VIB, VIC, VII, VIIA, VIII, VIIIA, IX, and IXA as follows:

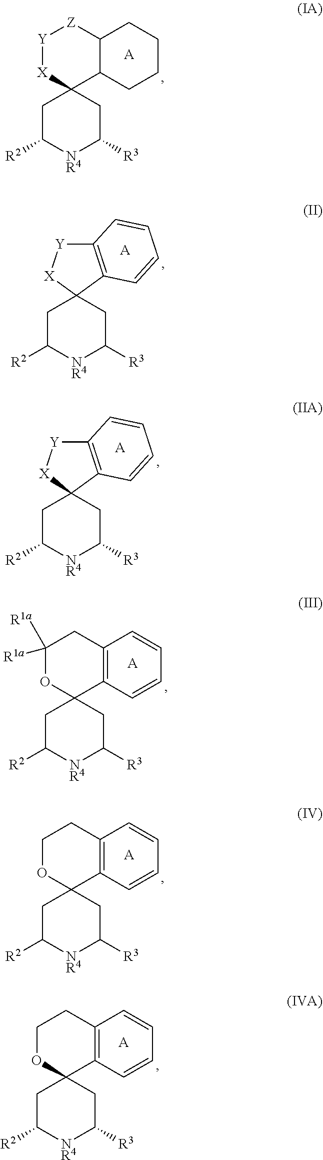

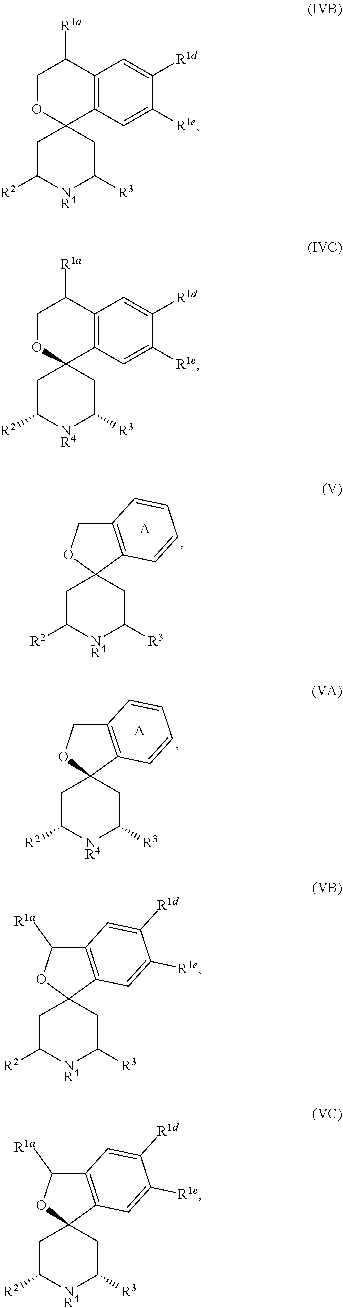

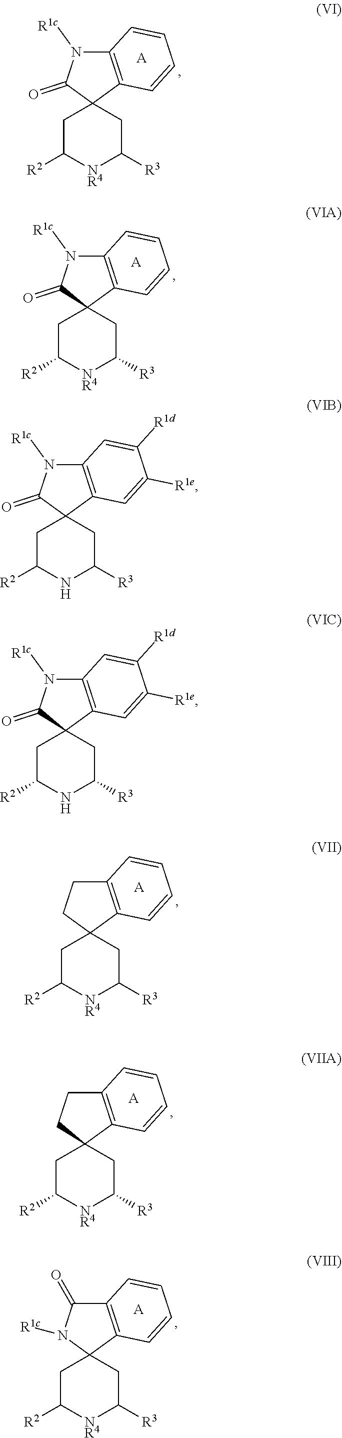

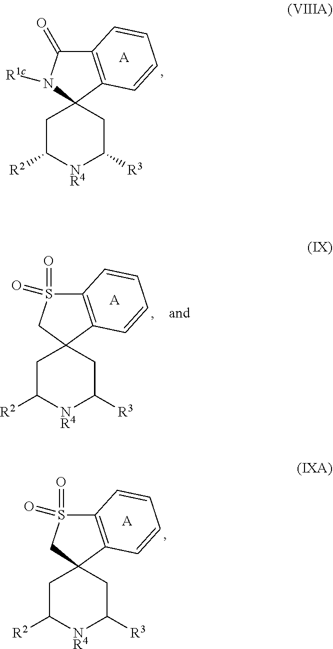

wherein all variables are as defined above for Formula I.

In one aspect of the disclosure, the compounds of Formulae I, IA, II, IIA, III, IV, IVA, IVB, IVC, V, VA, VB, VC, VI, VIA, VIB, VIC, VII, VIIA, VIII, VIIIA, IX, and IXA are chosen from Compounds 1 to 299, tautomers thereof, deuterated derivatives of those compounds and tautomers and pharmaceutically acceptable salts of any of the foregoing.

In some embodiments, the disclosure provides a pharmaceutical composition comprising at least one compound, tautomer, deuterated derivative, or pharmaceutically acceptable salt chosen from compounds of Formulae I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds or tautomers, and pharmaceutically acceptable salts of any of the foregoing. In some embodiments, the pharmaceutical composition may comprise at least one compound chosen from Compounds 1 to 299, tautomers thereof, deuterated derivatives of those compounds or tautomers, and pharmaceutically acceptable salts of any of the foregoing. These compositions may further include at least one additional active pharmaceutical ingredient and/or at least one carrier.

Another aspect of the disclosure provides methods of treating an APOL1-mediated disease comprising administering to a subject in need thereof, at least one compound, tautomer, deuterated derivative, or pharmaceutically acceptable salt chosen from compounds of Formulae I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt. In some embodiments, the methods comprise administering at least one compound, tautomer, deuterated derivative, or pharmaceutically acceptable salt chosen from Compounds 1 to 299, tautomers thereof, deuterated derivatives of those compounds or tautomers, and pharmaceutically acceptable salts of any of the foregoing.

Another aspect of the disclosure provides methods of treating an APOL1-mediated cancer (such as, e.g., pancreatic cancer) comprising administering to a subject in need thereof, at least one compound, tautomer, deuterated derivative, or pharmaceutically acceptable salt chosen from compounds of Formulae I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt. In some embodiments, the methods comprise administering at least one compound, tautomer, deuterated derivative, or pharmaceutically acceptable salt chosen from Compounds 1 to 299, tautomers thereof, deuterated derivatives of those compounds or tautomers, and pharmaceutically acceptable salts of any of the foregoing.

Another aspect of the disclosure provides methods of treating APOL1-mediated kidney disease (such as, e.g., ESKD, FSGS and/or NDKD) comprising administering to a subject in need thereof, at least one compound, tautomer, deuterated derivative, or pharmaceutically acceptable salt chosen from compounds of Formulae I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt. In some embodiments, the methods comprise administering at least one compound, tautomer, deuterated derivative, or pharmaceutically acceptable salt chosen from Compounds 1 to 299, tautomers thereof, deuterated derivatives of those compounds or tautomers, and pharmaceutically acceptable salts of any of the foregoing.

In some embodiments, the methods of treatment include administration of at least one additional active agent to the subject in need thereof, either in the same pharmaceutical composition as the at least one compound, tautomer, deuterated derivative, or pharmaceutically acceptable salt chosen from compounds of Formulae I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds or tautomers, and pharmaceutically acceptable salts of any of the foregoing, or as separate compositions. In some embodiments, the methods comprise administering at least one compound, tautomer, deuterated derivative, or pharmaceutically acceptable salt chosen from Compounds 1 to 299, tautomers thereof, deuterated derivatives of those compounds or tautomers, and pharmaceutically acceptable salts of any of the foregoing with at least one additional active agent, either in the same pharmaceutical composition or in a separate composition.

Also provided are methods of inhibiting APOL1, comprising administering to a subject in need thereof, at least one compound, tautomer, deuterated derivative, or pharmaceutically acceptable salt chosen from compounds of Formulae I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt. In some embodiments, the methods of inhibiting APOL1 comprise administering at least one compound, tautomer, deuterated derivative, or pharmaceutically acceptable salt chosen from Compounds 1 to 299, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt.

### DETAILED DESCRIPTION

### Definitions

The term “APOL1,” as used herein, means apolipoprotein L1 protein and the term “APOL1” means apolipoprotein L1 gene.

The term “APOL1 mediated disease” refers to a disease or condition associated with aberrant APOL1 (e.g., certain APOL1 genetic variants; elevated levels of APOL1). In some embodiments, an APOL1 mediated disease is an APOL1 mediated kidney disease. In some embodiments, an APOL1 mediated disease is associated with patients having two APOL1 risk alleles, e.g., patients who are homozygous or compound heterozygous for the G1 or G2 alleles. In some embodiments, an APOL1 mediated disease is associated with patients having one APOL1 risk allele.

The term “APOL1 mediated kidney disease” refers to a disease or condition that impairs kidney function and can be attributed to APOL1. In some embodiments, APOL1 mediated kidney disease is associated with patients having two APOL1 risk alleles, e.g., patients who are homozygous or compound heterozygous for the G1 or G2 alleles. In some embodiments, the APOL1 mediated kidney disease is chosen from ESKD, NDKD, FSGS, HIV-associated nephropathy, arterionephrosclerosis, lupus nephritis, microalbuminuria, and chronic kidney disease. In some embodiments, the APOL1 mediated kidney disease is chronic kidney disease or proteinuria.

The term “FSGS,” as used herein, means focal segmental glomerulosclerosis, which is a disease of the podocyte (glomerular visceral epithelial cells) responsible for proteinuria and progressive decline in kidney function, and associated with 2 common APOL1 genetic variants (GI: S342G:1384M and G2: N388del:Y389del).

The term “NDKD,” as used herein, means non-diabetic kidney disease, which is characterized by severe hypertension and progressive decline in kidney function, and associated with 2 common APOL1 genetic variants (GI: S342G:I384M and G2: N388del:Y389del).

The terms “ESKD” and “ESRD” are used interchangeably herein to refer to end stage kidney disease or end stage renal disease. ESKD/ESRD is the last stage of kidney disease, i.e., kidney failure, and means that the kidneys have stopped working well enough for the patient to survive without dialysis or a kidney transplant. In some embodiments, ESKD/ESRD is associated with two APOL1 risk alleles.

The term “compound,” when referring to a compound of this disclosure, refers to a collection of molecules having an identical chemical structure unless otherwise indicated as a collection of stereoisomers (for example, a collection of racemates, a collection of cis/trans stereoisomers, or a collection of (E) and (Z) stereoisomers), except that there may be isotopic variation among the constituent atoms of the molecules. Thus, it will be clear to those of skill in the art that a compound represented by a particular chemical structure containing indicated deuterium atoms will also contain lesser amounts of isotopologues having hydrogen atoms at one or more of the designated deuterium positions in that structure. The relative amount of such isotopologues in a compound of this disclosure will depend upon a number of factors including the isotopic purity of reagents used to make the compound and the efficiency of incorporation of isotopes in the various synthesis steps used to prepare the compound. However, as set forth above, the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

As used herein, “optionally substituted” is interchangeable with the phrase “substituted or unsubstituted.” In general, the term “substituted,” whether preceded by the term “optionally” or not, refers to the replacement of hydrogen radicals in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. Combinations of substituents envisioned by this disclosure are those that result in the formation of stable or chemically feasible compounds.

The term “isotopologue” refers to a species in which the chemical structure differs from a reference compound only in the isotopic composition thereof. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C or ¹⁴C, are within the scope of this disclosure.

Unless otherwise indicated, structures depicted herein are also meant to include all isomeric forms of the structures, e.g., racemic mixtures, cis/trans isomers, geometric (or conformational) isomers, such as (Z) and (E) double bond isomers, and (Z) and (E) conformational isomers. Therefore, geometric and conformational mixtures of the present compounds are within the scope of the disclosure. Unless otherwise stated, all tautomeric forms of the compounds of the disclosure are within the scope of the disclosure.

The term “tautomer,” as used herein, refers to one of two or more isomers of compound that exist together in equilibrium, and are readily interchanged by migration of an atom, e.g., a hydrogen atom, or group within the molecule.

“Stereoisomer,” as used herein, refers to enantiomers and diastereomers.

As used herein, “deuterated derivative” refers to a compound having the same chemical structure as a reference compound, but with one or more hydrogen atoms replaced by a deuterium atom (“D” or “²H”). It will be recognized that some variation of natural isotopic abundance occurs in a synthesized compound depending on the origin of chemical materials used in the synthesis. The concentration of naturally abundant stable hydrogen isotopes, notwithstanding this variation, is small and immaterial as compared to the degree of stable isotopic substitution of deuterated derivatives described herein. Thus, unless otherwise stated, when a reference is made to a “deuterated derivative” of a compound of the disclosure, at least one hydrogen is replaced with deuterium at well above its natural isotopic abundance (which is typically about 0.015%). In some embodiments, the deuterated derivatives of the disclosure have an isotopic enrichment factor for each deuterium atom, of at least 3500 (52.5% deuterium incorporation at each designated deuterium), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), or at least 6600 (99% deuterium incorporation).

The term “isotopic enrichment factor,” as used herein, means the ratio between the isotopic abundance and the natural abundance of a specified isotope.

The term “alkyl” or “aliphatic,” as used herein, means a straight-chain (i.e., linear or unbranched) or branched, substituted or unsubstituted hydrocarbon chain that is completely saturated. Unless otherwise specified, alkyl groups contain 1 to 20 alkyl carbon atoms. In some embodiments, alkyl groups contain 1 to 10 aliphatic carbon atoms. In some embodiments, alkyl groups contain 1 to 8 aliphatic carbon atoms. In some embodiments, alkyl groups contain 1 to 6 alkyl carbon atoms. In some embodiments, alkyl groups contain 1 to 4 alkyl carbon atoms, in other embodiments, alkyl groups contain 1 to 3 alkyl carbon atoms, and in yet other embodiments, alkyl groups contain 1 or 2 alkyl carbon atoms. In some embodiments, alkyl groups are linear or straight-chain or unbranched. In some embodiments, alkyl groups are branched.

The terms “cycloalkyl” and “cyclic alkyl,” as used herein, refer to a monocyclic C₃₋₈ hydrocarbon or a spirocyclic, fused, or bridged bicyclic or tricyclic Cs-14 hydrocarbon that is completely saturated, wherein any individual ring in said bicyclic ring system has 3 to 7 members. In some embodiments, the cycloalkyl is a C₃ to C₁₂ cycloalkyl. In some embodiments, the cycloalkyl is a C₃ to Cs cycloalkyl. In some embodiments, the cycloalkyl is a C₃ to C₆ cycloalkyl. Non-limiting examples of monocyclic cycloalkyls include cyclopropyl, cyclobutyl, cyclopentanyl, and cyclohexyl.

The terms “carbocyclyl” or “cycloaliphatic,” as used herein, encompass the terms “cycloalkyl” or “cyclic alkyl,” and refer to a monocyclic C₃₋₈ hydrocarbon or a spirocyclic, fused, or bridged bicyclic or tricyclic C₈₋₁₄ hydrocarbon that is completely saturated, or is partially saturated as in it contains one or more units of unsaturation but is not aromatic, wherein any individual ring in said bicyclic ring system has 3 to 7 members. Bicyclic carbocyclyls include combinations of a monocyclic carbocyclic ring fused to a phenyl. In some embodiments, the carbocyclyl is a C₃ to C₁₂ carbocyclyl. In some embodiments, the carbocyclyl is a C₃ to C₁₀ carbocyclyl. In some embodiments, the carbocyclyl is a C₃ to C₈ carbocyclyl.

The term “heteroalkyl,” or “heteroaliphatic,” as used herein, means an alkyl or aliphatic group as defined above, wherein one or two carbon atoms are independently replaced by one or more of oxygen, sulfur, nitrogen, phosphorus, or silicon.

The term “alkenyl,” as used herein, means a straight-chain (i.e., linear or unbranched) or branched hydrocarbon chain that contains one or more double bonds. In some embodiments, alkenyl groups are straight-chain. In some embodiments, alkenyl groups are branched.

The terms “heterocycle,” “heterocyclyl,” and “heterocyclic,” are used herein interchangeably to refer to non-aromatic (i.e., completely saturated or partially saturated as in it contains one or more units of unsaturation but is not aromatic), monocyclic, or spirocyclic, fused, or bridged bicyclic or tricyclic ring systems in which one or more ring members is an independently chosen heteroatom. Bicyclic heterocyclyls include the following combinations of monocyclic rings: a monocyclic heteroaryl fused to a monocyclic heterocyclyl; a monocyclic heterocyclyl fused to another monocyclic heterocyclyl; a monocyclic heterocyclyl fused to phenyl; a monocyclic heterocyclyl fused to a monocyclic carbocyclyl/cycloalkyl; and a monocyclic heteroaryl fused to a monocyclic carbocyclyl/cycloalkyl.

In some embodiments, the heterocycle comprises a ring atom substituted with one or more oxo groups (such as, e.g., a C═O group, a S═O group, or a SO₂ group).

In some embodiments, the “heterocycle,” “heterocyclyl,” “heterocycloaliphatic,” or “heterocyclic” group has 3 to 14 ring members in which one or more ring members is a heteroatom independently chosen from oxygen, sulfur, nitrogen, silicon, and phosphorus. In some embodiments, each ring in a bicyclic or tricyclic ring system contains 3 to 7 ring members. In some embodiments, the heterocycle has at least one unsaturated carbon-carbon bond. In some embodiments, the heterocycle has at least one unsaturated carbon-nitrogen bond. In some embodiments, the heterocycle has one heteroatom independently chosen from oxygen, sulfur, nitrogen, silicon, and phosphorus, the quaternized form of any basic nitrogen or; a substitutable nitrogen of a heterocyclic ring, for example, N (as in 3,4-dihydro-2H-pyrrolyl), NH (as in pyrrolidinyl) or NR⁺ (as in N-substituted pyrrolidinyl)). In some embodiments, the heterocycle has one heteroatom that is a nitrogen atom. In some embodiments, the heterocycle has one heteroatom that is an oxygen atom. In some embodiments, the heterocycle has two heteroatoms that are each independently chosen from nitrogen and oxygen. In some embodiments, the heterocycle has three heteroatoms that are each independently chosen from nitrogen and oxygen. In some embodiments, the heterocyclyl is a 3- to 12-membered heterocyclyl. In some embodiments, the heterocyclyl is a 3- to 10-membered heterocyclyl. In some embodiments, the heterocyclyl is a 3- to 8-membered heterocyclyl. In some embodiments, the heterocyclyl is a 5- to 10-membered heterocyclyl. In some embodiments, the heterocyclyl is a 5- to 8-membered heterocyclyl. In some embodiments, the heterocyclyl is a 5- or 6-membered heterocyclyl. Non-limiting examples of monocyclic heterocyclyls include piperidinyl, piperazinyl, tetrahydropyranyl, azetidinyl, tetrahydrothiophenyl 1,1-dioxide, and the like.

The term “unsaturated,” as used herein, means that a moiety has one or more units or degrees of unsaturation. Unsaturation is the state in which not all of the available valence bonds in a compound are satisfied by substituents and thus the compound contains double or triple bonds.

The term “alkoxy” or “thioalkyl,” as used herein, refers to an alkyl group, as previously defined, wherein one carbon of the alkyl group is replaced by an oxygen (“alkoxy”) or sulfur (“thioalkyl”) atom, respectively, provided that the oxygen and sulfur atoms are linked between two carbon atoms. A “cyclic alkoxy” refers to a monocyclic, spirocyclic, bicyclic, bridged bicyclic, tricyclic, or bridged tricyclic hydrocarbon that contains at least one alkoxy group, but is not aromatic. Non-limiting examples of cyclic alkoxy groups include tetrahydropyranyl, tetrahydrofuranyl, oxetanyl, 8-oxabicyclo[3.2.1]octanyl, and oxepanyl.

The terms “haloalkyl,” “haloalkenyl,” and “haloalkoxy,” as used herein, mean a linear or branched alkyl, alkenyl, or alkoxy, respectively, which is substituted with one or more halogen atoms. Non-limiting examples of haloalkyl groups include —CHF₂, —CH₂F, —CF₃, —CF₂—, and perhaloalkyls, such as —CF₂CF₃. Non-limiting examples of haloalkoxy groups include —OCHF₂, —OCH₂F, —OCF₃, and —OCF₂.

The term “halogen” includes F, Cl, Br, and I, i.e., fluoro, chloro, bromo, and iodo, respectively.

The term “aminoalkyl” means an alkyl group which is substituted with or contains an amino group.

As used herein, an “amino” refers to a group which is a primary, secondary, or tertiary amine.

As used herein, a “carbonyl” group refers to C═O.

As used herein, a “cyano” or “nitrile” group refer to —C≡N.

As used herein, a “hydroxy” group refers to —OH.

As used herein, a “thiol” group refers to —SH.

As used herein, “tert” and “t-” each refer to tertiary.

As used herein, “aromatic groups” or “aromatic rings” refer to chemical groups that contain conjugated, planar ring systems with delocalized pi electron orbitals comprised of [4n+2] p orbital electrons, wherein n is an integer ranging from 0 to 6. Non-limiting examples of aromatic groups include aryl and heteroaryl groups.

The term “aryl,” used alone or as part of a larger moiety as in “arylalkyl,” “arylalkoxy,” or “aryloxyalkyl,” refers to monocyclic or spirocyclic, fused, or bridged bicyclic or tricyclic ring systems having a total of five to fourteen ring members, wherein every ring in the system is an aromatic ring containing only carbon atoms and wherein each ring in a bicyclic or tricyclic ring system contains 3 to 7 ring members. Non-limiting examples of aryl groups include phenyl (C₆) and naphthyl (C₁₀) rings.

The term “heteroaryl,” used alone or as part of a larger moiety as in “heteroarylalkyl” or “heteroarylalkoxy,” refers to monocyclic or spirocyclic, fused, or bridged bicyclic or tricyclic ring systems having a total of five to fourteen ring members, wherein at least one ring in the system is aromatic, wherein at least one ring in the system contains one or more heteroatoms, and wherein each ring in a bicyclic or tricyclic ring system contains 3 to 7 ring members. Bicyclic heteroaryls include the following combinations of monocyclic rings: a monocyclic heteroaryl fused to another monocyclic heteroaryl; and a monocyclic heteroaryl fused to a phenyl. In some embodiments, heteroaryl groups have one or more heteroatoms chosen from nitrogen, oxygen, and sulfur. In some embodiments, heteroaryl groups have one heteroatom. In some embodiments, heteroaryl groups have two heteroatoms. In some embodiments, heteroaryl groups are monocyclic ring systems having five ring members. In some embodiments, heteroaryl groups are monocyclic ring systems having six ring members. In some embodiments, the heteroaryl is a 3- to 12-membered heteroaryl. In some embodiments, the heteroaryl is a 3- to 10-membered heteroaryl. In some embodiments, the heteroaryl is a 3-to 8-membered heteroaryl. In some embodiments, the heteroaryl is a 5- to 10-membered heteroaryl. In some embodiments, the heteroaryl is a 5- to 8-membered heteroaryl. In some embodiments, the heteroaryl is a 5- or 6-membered heteroaryl. Non-limiting examples of monocyclic heteroaryls are pyridinyl, pyrimidinyl, thiophenyl, thiazolyl, isoxazolyl, etc.

Non-limiting examples of useful protecting groups for nitrogen-containing groups, such as amine groups, include, for example, t-butyl carbamate (Boc), benzyl (Bn), tetrahydropyranyl (THP), 9-fluorenylmethyl carbamate (Fmoc) benzyl carbamate (Cbz), acetamide, trifluoroacetamide, triphenylmethylamine, benzylideneamine, and p-toluenesulfonamide. Methods of adding (a process generally referred to as “protecting”) and removing (process generally referred to as “deprotecting”) such amine protecting groups are well-known in the art and available, for example, in P. J. Kocienski, Protecting Groups, Thieme, 1994, which is hereby incorporated by reference in its entirety and in Greene and Wuts, Protective Groups in Organic Synthesis, 3rd Edition (John Wiley & Sons, New York, 1999) and 4^{th} Edition (John Wiley & Sons, New Jersey, 2014).

Non-limiting examples of suitable solvents that may be used in this disclosure include, but are not limited to, water, methanol (MeOH), ethanol (EtOH), dichloromethane or “methylene chloride” (CH₂Cl₂), toluene, acetonitrile (MeCN), dimethylformamide (DMF), dimethyl sulfoxide (DMSO), methyl acetate (MeOAc), ethyl acetate (EtOAc), heptane, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in this disclosure include, but are not limited to, 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH) and sodium methoxide (NaOMe; NaOCH₃).

The disclosure includes pharmaceutically acceptable salts of the disclosed compounds. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response, and the like, and are commensurate with a reasonable benefit/risk ratio. A “pharmaceutically acceptable salt” means any non-toxic salt that, upon administration to a recipient, is capable of providing, either directly or indirectly, a compound of this disclosure. Suitable pharmaceutically acceptable salts are, for example, those disclosed in S. M. Berge, et al. J. Pharmaceutical Sciences, 1977, 66, 1 to 19.

Acids commonly employed to form pharmaceutically acceptable salts include inorganic acids such as hydrogen bisulfide, hydrochloric acid, hydrobromic acid, hydroiodic acid, sulfuric acid, and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate, and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄ alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate, and aryl sulfonate. Other suitable, non-limiting examples of pharmaceutically acceptable salts include besylate and glucosamine salts.

The terms “patient” and “subject” are used interchangeably herein and refer to an animal, including a human.

The terms “effective dose” and “effective amount” are used interchangeably herein and refer to that amount of compound that produces a desired effect for which it is administered (e.g., improvement in a symptom of FSGS and/or NDKD, lessening the severity of FSGS and/NDKD or a symptom of FSGS and/or NDKD, and/or reducing progression of FSGS and/or NDKD or a symptom of FSGS and/or NDKD). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the term “treatment” and its cognates refer to slowing or stopping disease progression. “Treatment” and its cognates as used herein, include, but are not limited to, the following: complete or partial remission, lower risk of kidney failure (e.g., ESRD), and disease-related complications (e.g., edema, susceptibility to infections, or thrombo-embolic events). Improvements in or lessening the severity of any of these symptoms can be readily assessed according to methods and techniques known in the art or subsequently developed.

The terms “about” and “approximately,” when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent.

The at least one compound, tautomer, deuterated derivative, or pharmaceutically acceptable salt chosen from compounds of Formulae I and II, tautomers thereof, deuterated derivatives of those compounds and tautomers, and pharmaceutically acceptable salts of any of the foregoing, may be administered once daily, twice daily, or three times daily, for example, for the treatment of AMKD, including FSGS and/or NDKD. In some embodiments, at least one compound chosen from Compounds 1 to 299, tautomers thereof, deuterated derivatives of those compounds and tautomers, and pharmaceutically acceptable salts of any of the foregoing may be administered once daily, twice daily, or three times daily, for example, for the treatment of AMKD, including FSGS and/or NDKD. In some embodiments, at least one compound, tautomer, deuterated derivative, or pharmaceutically acceptable salt chosen from compounds of Formulae I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds and tautomers, and pharmaceutically acceptable salts of any of the foregoing is administered once daily. In some embodiments, at least one compound chosed from Compounds 1 to 299, tautomers thereof, deuterated derivatives of those compounds and tautomers, and pharmaceutically acceptable salts of any of the foregoing is administered once daily. In some embodiments, at least one compound, tautomer, deuterated derivative, or pharmaceutically acceptable salt chosen from compounds of Formulae I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds and tautomers, and pharmaceutically acceptable salts of any of the foregoing is administered twice daily. In some embodiments, at least one compound chosed from Compounds 1 to 299, tautomers thereof, deuterated derivatives of those compounds and tautomers, and pharmaceutically acceptable salts of any of the foregoing is administered twice daily. In some embodiments, at least one compound, tautomer, deuterated derivative, or pharmaceutically acceptable salt chosen from compounds of Formulae I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds and tautomers, and pharmaceutically acceptable salts of any of the foregoing is administered three times daily. In some embodiments, at least one compound chosed from Compounds 1 to 299, tautomers thereof, deuterated derivatives of those compounds and tautomers, and pharmaceutically acceptable salts of any of the foregoing is administered three times daily.

In some embodiments, 2 mg to 1500 mg or 5 mg to 1000 mg of at least one compound chosen from Formulae I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds or tautomers, and pharmaceutically acceptable salts of any of the foregoing is administered once daily, twice daily, or three times daily. In some embodiments, 2 mg to 1500 mg or 5 mg to 1000 mg of at least one compound chosen from Compounds 1 to 299, tautomera thereof, deuterated derivative of those compounds and tautomers, and pharmaceutically acceptable salts of any of the foregoing is administered once daily, twice daily, or three times daily.

One of ordinary skill in the art would recognize that, when an amount of compound is disclosed, the relevant amount of a pharmaceutically acceptable salt form of the compound is an amount equivalent to the concentration of the free base of the compound. The amounts of the compounds, pharmaceutically acceptable salts, solvates, and deuterated derivatives disclosed herein are based upon the free base form of the reference compound. For example, “1000 mg of at least one compound or pharmaceutically acceptable salt chosen from compounds of Formula I and pharmaceutically acceptable salts thereof” includes 1000 mg of a compound of Formula I and a concentration of a pharmaceutically acceptable salt of compounds of Formula I equivalent to 1000 mg of a compound of Formula I.

As used herein, the term “ambient conditions” means room temperature, open air condition, and uncontrolled humidity condition.

### Compounds and Compositions

In some embodiments, at least one compound chosen from I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds and tautomers, and pharmaceutically acceptable salt of any of the foregoing may be employed in the treatment of AMKD, including FSGS and NDKD. In some embodiments, the compound of I, IA, II, IIA, III, IV, IVA, IVB, IVC, V, VA, VB, VC, VI, VIA, VIB, VIC, VII, VIIA, VIII, VIIIA, IX, and IXA, may be chosen from Compounds 1 to 299, tautomers thereof, deuterated derivatives of those compounds and tautomers, and pharmaceutically acceptable salts of any of the foregoing. In some embodiments, a pharmaceutical composition comprising at least one compound chosen from I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds and tautomers, and pharmaceutically acceptable salt of any of the foregoing, may be employed in the treatment of AMKD, including FSGS and NDKD. In some embodiments the pharmaceutical composition comprises at least one compound chosen from Compounds 1 to 299, tautomers thereof, deuterated derivatives of those compounds and tautomers, and pharmaceutically acceptable salt of any of the foregoing.

In some embodiments of Formula I.

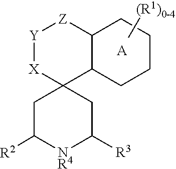

Ring A is an unsubstituted phenyl group. In some embodiments, Ring A is phenyl substituted with 1 or 2 R¹ groups and each R¹ is independently chosen from halogen, cyano, —CH₃, —CH₂CH₃, —CH₂CF₂, —CFCF₂, —CF₂, —CF₃, —OCH₃, —OCF₂, and —OCF₃. In some embodiments, Ring A is phenyl substituted with 1 or 2 R¹ groups and each R¹ is independently chosen from F and Cl.

In some embodiments of Formula I, X is chosen from —C(O)—, —O—, —S—, —CR^{1a}R^{1b}—, and —NR^{1c}—. In some embodiments of Formula I, X is chosen from —CH₂— and —NH— (i.e., R^{1a}, R^{1b}, and R^{1c} are each hydrogen). In some embodiments of Formula I, X is —CR^{1a}R^{1b}—, wherein R^{1a} is hydrogen and R^{1b} is chosen from R¹ groups. In some embodiments of Formula I, X is —CHOH— (i.e., wherein R^{1a} is hydrogen, R^{1b} is R¹, and R¹ is —OH). In some embodiments of Formula I, X is —O—. In some embodiments of Formula I, X is —C(O)—.

In some embodiments of Formula I, Y is chosen from —CR^{1a}R^{1b}— and —NR^{1c}—. In some embodiments of Formula I, Y is —CR^{1a}R^{1b}, wherein R^{1a} and R^{1b} are hydrogen. In some embodiments of Formula I, Y is —CR^{1a}R^{1b}, wherein R^{1a} and R^{1b} are each R¹, and R¹ is —CH₃. In some embodiments of Formula I, Y is —CR^{1a}R^{1b}, wherein R^{1a} is hydrogen and R^{1b} is chosen from R¹ groups. In some embodiments of Formula I, Y is —CR^{1a}R^{1b}, wherein R^{1a} is hydrogen, R^{1b} is R¹, and R¹ is chosen from

—OH, —CH₃, —C(O)NH₂, C(O)NHCH₃. In some embodiments of Formula I, Y is —CR^{1a}R^{1b}, wherein R^{1a} and R^{1b} are R¹, and R¹ is chosen from —OH and —CH₃.

In some embodiments of Formula I, X is chosen from —C(O)— and —CR^{1a}R^{1b}—, and Y is —NR^{1c}—, wherein R^{1c} is hydrogen. In some embodiments of Formula I, X is chosen from —C(O)— and —CR^{1a}R^{1b}—, and Y is —NR^{1c}—, wherein R^{1c} is chosen from R¹ groups. In some embodiments of Formula I, X is chosen from —C(O)— and —CR^{1a}R^{1b}—, and Y is —NR^{1c}—, wherein R^{1c} is R¹, and R¹ is chosen from C₁-C₆ alkyl and C₁-C₆ alkoxy optionally substituted with I to 3 halogen groups. In some embodiments of Formula I, X is chosen from —C(O)— and —CR^{1a}R^{1b}—, and Y is —NR^{1c}—, wherein R^{1c} is R¹, and R¹ is chosen from —CH₃, —CH₂CH₃, —CH₂CH₂OH, —CH₂CF₂, —CH₂CF₃, and —CH₂CH₂OCH₃. In some embodiments of Formula I, Y is —NR^{1c}—, wherein R^{1c} is R¹, and R¹ is chosen from 4- to 6-membered heterocyclyl. In some embodiments of Formula I, Y is is —NR^{1c}—, wherein R^{1c} is R¹, and R¹ is

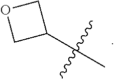 [SMILES: *C1COC1]

In some embodiments of Formula I, Y is —CH(C(O)NH₂)— In some embodiments of Formula I, Y is —CH(C(O)NHCH₃)—. In some embodiments of Formula I, Y is —CH(CH₃)₂—. In some embodiments of Formula I, Y is —CH₂—. In some embodiments of Formula I, Y is —CH(OH)—. In some embodiments of Formula I, Y is —NH—. In some embodiments of Formula I, Y is

—N(CH₃)—. In some embodiments of Formula I, Y is —N(C(O)CH₃)—.

In some embodiments of Formula I, Z is a bond (i.e., Y is connected directly to Ring A). In some embodiments of Formula I, and Z is chosen from —CR^{1a}R^{1b}—. In some embodiments of Formula I, and Z is —CR^{1a}R^{1b}—, wherein R^{1a} and R^{1b} are hydrogen. In some embodiments of Formula I, and Z is —CR^{1a}R^{1b}—, wherein R^{1a} and R^{1b} are fluorine. In some embodiments of Formula I, and Z is —CR^{1a}R^{1b}—, wherein R^{1a} is hydrogen and R^{1b} is —OH.

In some embodiments of Formula I, R² and R³ are independently chosen from hydrogen and CH₃. In some embodiments of Formula I, R² and R³ are both hydrogen. In some embodiments of Formula I, one of R² and R³ is hydrogen and the other is CH₃.

In some embodiments of Formula I, including embodiments of each of Formulae IA, II, IIA, III, IV, IVA, IVB, IVC, V, VA, VB, VC, VI, VIA, VIB, VIC, VII, VIIA, VIII, VIIIA, IX, and IXA, R⁴ is chosen from C₁-C₆ alkyl, optionally substituted with 1 to 5 groups independently chosen from halogen, cyano, —OH, —NH₂, —NH(C₁-C₄ alkyl), —N(C₁-C₄ alkyl)₂, —C(═O)NH₂,

—C(═O)(C₁-C₄ alkyl), —C(═O)OH, —C(═O)O(C₁-C₄ alkyl), —C(═O)NH(C₁-C₄ alkyl), —C(═O)N(C₁-C₄ alkyl)₂, C₁-C₄ alkoxy, C₃-C₆ carbocyclyl, C₆ aryl (optionally substituted with 1 to 3 groups independently chosen from halogen and C₁-C₄ haloalkyl groups), —O—(C₆ aryl) (optionally substituted with 1 to 3 groups independently chosen from halogen and C₁-C₄ haloalkyl groups), 5- to 10-membered heterocyclyl, and 5- to 10-membered heteroaryl groups. In some embodiments of Formula I, including embodiments of each of Formulae IA, II, IIA, III, IV, IVA, IVB, IVC, V, VA, VB, VC, VI, VIA, VIB, VIC, VII, VIIA, VIII, VIIIA, IX, and IXA, R⁴ is chosen from C₁-C₆ alkyl substituted with 1 to 2 groups independently chosen from —OH, phenyl, and phenyl further substituted with halogen. In some embodiments of Formula I, R⁴ is chosen from C₁-C₆ alkyl substituted with —OH and phenyl. In some embodiments of Formula I, including embodiments of each of Formulae IA, II, IIA, III, IV, IVA, IVB, IVC, V, VA, VB, VC, VI, VIA, VIB, VIC, VII, VIIA, VIII, VIIIA, IX, and IXA, R⁴ is chosen from C₁-C₆ alkyl substituted with —OH and phenyl substituted with C₁. In some embodiments of Formula I, R⁴ is chosen from C₁-C₆ alkyl substituted with —OH and phenyl substituted with 2F. In some embodiments of Formula I, R⁴ is chosen from —C(═O)O(C₁-C₄ alkyl).

In some embodiments of Formula I, including embodiments of each of Formulae IA, II, IIA, III, IV, IVA, IVB, IVC, V, VA, VB, VC, VI, VIA, VIB, VIC, VII, VIIA, VIII,

VIIIA, IX, and IXA, R⁴ is chosen from

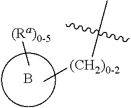

groups. In some embodiments of Formula I, including embodiments of each of Formulae IA, II, IIA, III, IV, IVA, IVB, IVC, V, VA, VB, VC, VI, VIA, VIB, VIC, VII, VIIA, VIII, VIIIA, IX, and IXA, R⁴ is chosen from

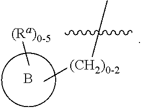

In some embodiments, Ring B is chosen from

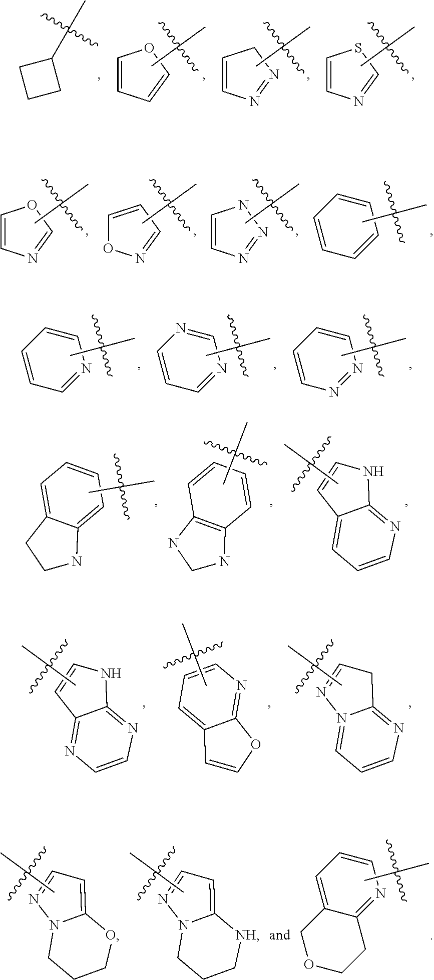

In some embodiments of Formula I, including embodiments of each of Formulae IA, II, IIA, III, IV, IVA, IVB, IVC, V, VA, VB, VC, VI, VIA, VIB, VIC, VII, VIIA, VIII, VIIIA, IX, and IXA, Ring B is unsubstituted. In some embodiments, Ring B is substituted with 1 R^{a} group. In some embodiments, Ring B is substituted with 2 R^{a} groups. In some embodiments, Ring B is substituted with 3 R^{a} groups. In some embodiments, Ring B is substituted with 4 R^{a} groups.

In some embodiments of Formula I, including embodiments of each of Formulae IA, II, IIA, III, IV, IVA, IVB, IVC, V, VA, VB, VC, VI, VIA, VIB, VIC, VII, VIIA, VIII, VIIIA, IX, and IXA, each R^{a} is independently selected from chosen from halogen, cyano, oxo, C₁-C₈ alkyl, C₁-C₆ haloalkyl, C₁-C₆ alkoxy, C₁-C₆ haloalkoxy, C₃-C₁₂ carbocyclyl, C₆ and C₁₀ aryl, 3- to 12-membered heterocyclyl, 5- to 10-membered heteroaryl, —C(═O)NR^{h}Rⁱ, —C(═O)OR^{k}, —NR^{h}Rⁱ,
—NR^{h}C(═O)R^{k}, —NR^{h}C(O)OR^{k}, —NR^{h}S(═O)ₚR^{k}, —S(═O)ₚR^{k}, and —S(═O)ₚNR^{h}Rⁱ groups,
wherein:
   the C₁-C₈ alkyl, the C₁-C₆ haloalkyl, the C₁-C₆ alkoxy, and the C₂-C₅ alkenyl of R^{a} are each optionally substituted with 1 to 3 groups independently chosen from —C(═O)NR^{h}Rⁱ, =NOR^{k}, —NR^{h}Rⁱ, —NR^{h}C(═O)NRⁱR^{j}, —NR^{h}S(═O)ₚR^{k}, —OR^{k}, —S(═O)ₚR^{k}, —S(═O)ₚNR^{h}Rⁱ, C₃-C₆ carbocyclyl (optionally substituted with 1 to 3 R^{m} groups), 4- to 10-membered heterocyclyl (optionally substituted with 1 to 3 R^{m} groups), and 5- to 10-membered heteroaryl (optionally substituted with 1 to 3 R^{m} groups) groups;
   the C₃-C₁₂ carbocyclyl, the 3- to 12-membered heterocyclyl, the C₆ and C₁₀ aryl, and the 5- to 10-membered heteroaryl of R^{a} are each optionally substituted with 1 to 3 groups independently chosen from oxo, halogen, cyano, C₁-C₆ alkyl (optionally substituted with 1 to 3 R^{m} groups), —OR^{k}, —S(═O)ₚR^{k}, —S(═O)ₚNR^{h}Rⁱ, and 5- to 10-membered heterocyclyl groups, wherein:
      R^{h}, Rⁱ, and R^{j}, for each occurrence, are each independently chosen from hydrogen, C₁-C₆ alkyl, 5- to 10-membered heteroaryl (optionally substituted with 1 to 3 R^{m} groups), and 5- to 10-membered heterocyclyl (optionally substituted with 1 to 3 R^{m} groups) groups, wherein:
         the C₁-C₆ alkyl of any one of R^{h}, Rⁱ, and R^{j} is optionally substituted with 1 to 3 groups independently chosen from halogen, cyano, —OH, C₃-C₆ carbocyclyl (optionally substituted with 1 to 3 R^{m} groups);
      R^{k}, for each occurrence, is independently chosen from hydrogen, C₁-C₆ alkyl, C₃-C₆ carbocyclyl, and 5- to 10-membered heteroaryl groups, wherein:
         the C₁-C₆ alkyl of any one of R^{k} is optionally substituted with 1 to 5 groups independently chosen from halogen, cyano, —NH₂, and —OH; and
         the 5- to 10-membered heteroaryl and 5- to 10-membered heterocyclyl of any one of R^{k} are each optionally substituted with 1 to 3 groups independently chosen from halogen, cyano, —OH, C₁-C₄ alkyl (optionally substituted by 1 to 3 —OH groups) C₁-C₄ haloalkyl, C₃-C₆ cycloalkyl, and C₁-C₄ alkoxy groups, wherein:
      R^{m}, for each occurrence, is independently chosen from halogen, cyano, oxo, —C(═O)NH₂, —NH₂, —C₁-C₆ alkyl, C₁-C₆ alkoxy, —OR^{k}, and C₃-C₆ cycloalkyl, wherein:
         the C₁-C₆ alkyl, the C₁-C₆ alkoxy, and the 5- to 10-membered heterocyclyl of any one of R^{m} is optionally substituted with 1 to 3 groups independently chosen from halogen, cyano, —OH, and C₁-C₄ alkoxy groups;
   p, for each occurrence, is an integer independently chosen from 1 and 2.

In some embodiments of Formula I, including embodiments of each of Formulae IA, II, IIA, III, IV, IVA, IVB, IVC, V, VA, VB, VC, VI, VIA, VIB, VIC, VII, VIIA, VIII, VIIIA, IX, and IXA, each R^{a} is independently chosen from
-halogen;
-oxo;
-cyano;
—C₁ haloalkoxy;
CH₃; —CF₂;
C₁ alkyl substituted with —OR^{k}, wherein R^{k} is —CH₃;
C₁ alkyl substituted with —C(═O)NR^{h}Rⁱ, wherein R^{h} and Rⁱ are both CH₃;
—C₁ alkyl substituted with —C(═O)NR^{h}Rⁱ, wherein R^{h} is hydrogen and Rⁱ is CH₃;
—C₁ alkyl substituted with —C(═O)NR^{h}Rⁱ, wherein R^{h} is hydrogen and Rⁱ is CH₃ substituted with C₃-C₆ carbocyclyl optionally substituted cyano;
—C₁ alkyl substituted with —C(═O)NR^{h}Rⁱ, wherein R^{h} is hydrogen and Rⁱ is CH₃ substituted with a 5-membered heteroaryl;
C₁ alkyl substituted with a cyclopropyl, which is further substituted with R^{m}, wherein R^{m} is selected from CH₃, CH₂OH, and CH₂C(═O)NH₂;
—C₁ alkyl substituted with a 4-membered heterocycle,

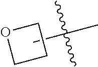

which is further substituted with R^{m}, wherein R^{m} is C(═O)NH₂;
—C₁ alkyl substituted with a 5-membered optionally substituted heterocycle;
—C₁ alkyl substituted with

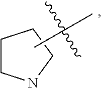

which is optionally substituted with oxo and CH₃;
—C₁ alkyl substituted with a 5-membered optionally substituted heteroaryl;
—C₁ alkyl substituted with

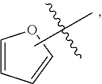

which is optionally substituted with —OH or C(═O)NH₂;
—C₁ alkyl substituted with

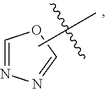

which is optionally substituted with —C₁ to —C₃ alkyl; —C₁ alkyl substituted with

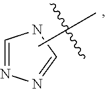

which is optionally substituted with N(CH₃)₂;
—C₁ alkyl substituted with

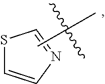

which is optionally substituted with 1 to 3 R^{m} groups independently selected from CH₃, CH₂OH, and C(═O)NH₂;
—C₁ alkyl substituted with a 6-membered optionally substituted heteroaryl;
—C₁ alkyl substituted with

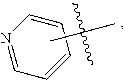

which is optionally substituted with 1 to 3 R^{m} groups independently selected from oxo, CH3, and CH₂OH;
—C₁ alkyl substituted with

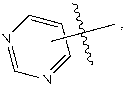

which is optionally substituted with 1 to 3 R^{m} groups independently selected from oxo, CH₃, —C₃ branched alkyl, cyclopropyl, and NHCH₃;
—C₁ alkyl substituted with

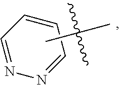

which is optionally substituted with 1 to 3 R^{m} groups independently selected from —OH, oxo, and C₁-C₃ alkyl;
—C₁ alkyl substituted with

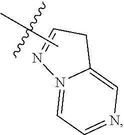

optionally substituted with 1 to 2 R^{m} groups independently selected from oxo and CH₃;
—C₁ alkyl substituted with

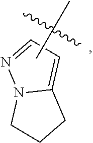

optionally substituted with 1 to 2 R^{m} groups independently selected from oxo and CH₃:
—C₁ alkyl substituted with

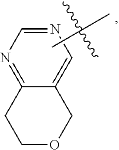

optionally substituted with 1 to 2 R^{m} groups;
C₁-C₃ alkoxy;
—C₂ alkyl;
—C₂ alkyl substituted with —OH and —C₃-C₆ carbocyclyl (optionally substituted with —OH);
—C₂ alkyl substituted with C₁ alkoxy;
—C₂ alkyl substituted with —OH;
—C₂ alkyl substituted with —NR^{h}Rⁱ, wherein Rh is hydrogen, and R is a 6-membered heteroaryl optionally substituted with an R^{m} group selected from —C₁-C₃ alkyl;
—C₂ alkyl substituted with ═NOR^{k}, wherein R^{k} is —C₂ alkyl;
—C₂ alkyl substituted with —NR^{h}S(═O)ₚR^{k}, wherein Rh is hydrogen, p is 2 and R^{k} is cyclopropyl;
—C₂ alkyl substituted with —NR^{h}C(═O)NRⁱR^{j}, wherein R^{h} and Rare hydrogen, and Rⁱ is —C₂ alkyl;
—C₂ alkyl substituted with S(═O)ₚR^{k} wherein p is 2 and R^{k} is CH₃;
—C₂ alkyl substituted with —S(═O)ₚNR^{h}Rⁱ, wherein p is 2 and R^{h} and Rⁱ both hydrogen;
—C₂ alkyl substituted with —S(═O)ₚNR^{h}Rⁱ, wherein R^{h} is hydrogen and Rⁱ is CH₃;
—C₂ alkyl substituted with —OH and 6 membered heterocycle (optionally further substituted with —OH);
—C₂ alkyl substituted with a 6-membered heterocycle,

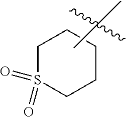

which is further substituted with R^{m}, wherein R^{m} is —OR^{k}, wherein R^{k} is —OH;
—C₂ alkyl substituted with —OH and optionally substituted

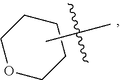

which is further substituted with OH;
—C₂ alkyl substituted with a 5-membered, optionally-substituted heteroaryl;
—C₂ alkyl substituted with 1 to 2 halogen groups and

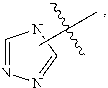

which is optionally substituted with C₁-C₃ alkyl;
—C₂ alkyl substituted with a 6-membered, optionally substituted heteroaryl;
—C₂ alkyl substituted with

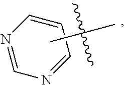

which is optionally substituted with oxo;
—C₃ alkyl optionally substituted with 1-2 —OH groups;
—C₃ haloalkyl substituted with —OH;
—C₃ carbocycle;
—C₃ alkyl substituted with 5-membered, optionally-substitute heteroaryl;
—C₃ alkyl substituted with

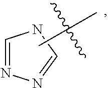

which is optionally substituted with oxo;
—C₃ alkyl substituted with 6-membered heteroaryl, optionally substituted with 1 to 2 oxo groups;
—C₃ alkyl substituted with

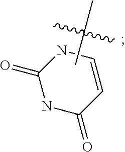

—C₄ alkyl substituted with two —OH groups;
—C₄ branched alkyl substituted with —OH;
—C₄ branched alkyl substituted with —NR^{h}S(═O)ₚR^{k}, wherein R^{h} is hydrogen, p is 2 and R^{k} is CH₃;
—C₄ branched alkoxy substituted with —NR^{h}Rⁱ, wherein wherein R^{h} and Rⁱ both hydrogen;
—C₅ branched alkyl substituted with two —OH groups;
—C₅ branched alkyl substituted with —S(═O)ₚNR^{h}Rⁱ, wherein p is 2 and R^{h} and Rⁱ both hydrogen;
—C₆ aryl, optionally substituted with CF₃;
—C(═O)OR^{k}, wherein R^{k} is CH₃;
—C(═O)NR^{h}Rⁱ, wherein wherein R^{h} is hydrogen and Rⁱ is CH₃;
—C(═O)NR^{h}Rⁱ, wherein wherein R^{h} and Rⁱ are both hydrogen;
—NR^{h}Rⁱ, wherein wherein R^{h} and Rⁱ both hydrogen;
NR^{h}Rⁱ, wherein R^{h} is hydrogen and R is CH₃;
—NR^{h}Rⁱ, wherein wherein R^{h} is hydrogen and Rⁱ is branched —C₄ alkyl substituted with —OH;
—NR^{h}C(═O)R^{k}, wherein R^{h} is hydrogen and R^{k} is CH₃;
—NR^{h}C(═O)R^{k}, wherein wherein R^{h} is hydrogen and R^{k} is a 5 membered heteroaryl,

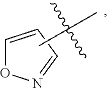

substituted with a group chosen from Cl and cyclopropyl;
—NR^{h}C(═O)R^{k}, wherein R^{h} is hydrogen and R^{k} is unsubstituted

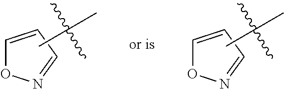

substituted with CH₃;
—NR^{h}S(═O)ₚR^{k}, wherein R^{h} is hydrogen, p is 2, and R^{k} is —C₂ alkyl;
—NR^{h}S(═O)ₚR^{k}, wherein R^{h} is hydrogen, p is 2, and R^{k} is cyclopropyl;
—NR^{h}S(═O)ₚR^{k}, wherein R^{h} is hydrogen, p is 2, and R^{k} is -cyano;
S(═O)ₚR^{k}, wherein R^{k} is CH₃; —S(═O)ₚNR^{h}Rⁱ, wherein p is 2, and R^{h} and Rⁱ, are both hydrogen; 4-membered heterocycle,

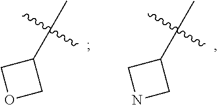 [SMILES: *C1COC1 ; *C1CNC1]

optionally substituted with S(O)₂CH₃;
5-membered heterocycle;

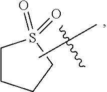

optionally substituted with CH₃;

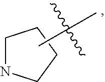

optionally substituted with oxo and CH₃;
6 membered heterocycle;
optionally substituted

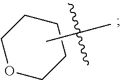

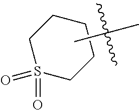

optionally substituted with —OH;
5-membered heteroaryl;
6 membered heteroaryl;

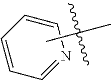

substituted with —C₁-C₃ alkyl

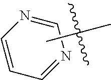

optionally substituted with —C₁-C₃ alkyl.

In some embodiments, the at least one compound, tautomer, deuterated derivative, or pharmaceutically acceptable salt of the disclosure is chosen from Compounds 1 to 299 depicted in Table 1, a tautomer thereof, a deuterated derivative of that compound or tautomer, or a pharmaceutically acceptable salt of any of the foregoing. A wavy line in a compound in Table 1 (i.e., [symbol] ) depicts a bond between two atoms and indicates a position of mixed stereochemistry for a collection of molecules, such as a racemic mixture, cis/trans isomers, or (E)/(Z) isomers. An asterisk adjacent to an atom (e.g., [symbol] ) in a compound in Table 1, indicates a chiral position in the molecule.

In some embodiments, the compound of Formula I is selected from the compounds presented in Table 1 below, tautomers of those compounds, deuterated derivatives of those compounds and tautomers, and pharmaceutically acceptable salts of any of the foregoing.

TABLE 1
Compounds 1 to 314
1

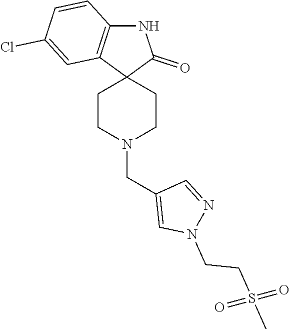 [SMILES: CS(=O)(=O)CCn1cc(CN2CCC3(CC2)C(=O)Nc2ccc(Cl)cc23)cn1]

2

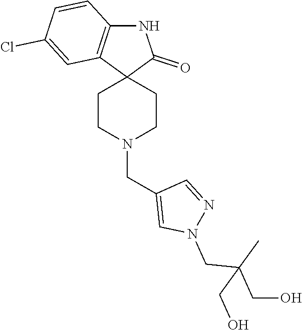 [SMILES: CC(CO)(CO)Cn1cc(CN2CCC3(CC2)C(=O)Nc2ccc(Cl)cc23)cn1]

3

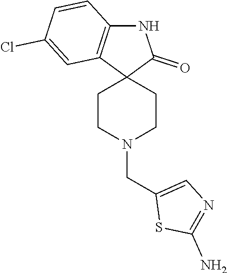 [SMILES: Nc1ncc(CN2CCC3(CC2)C(=O)Nc2ccc(Cl)cc23)s1]

4

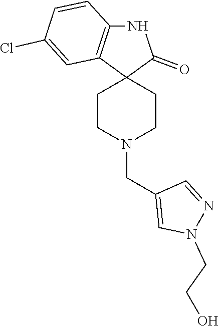 [SMILES: O=C1Nc2ccc(Cl)cc2C12CCN(Cc1cnn(CCO)c1)CC2]

5

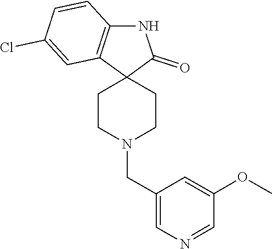 [SMILES: COc1cncc(CN2CCC3(CC2)C(=O)Nc2ccc(Cl)cc23)c1]

6

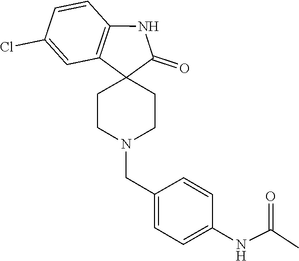 [SMILES: CC(=O)Nc1ccc(CN2CCC3(CC2)C(=O)Nc2ccc(Cl)cc23)cc1]

7

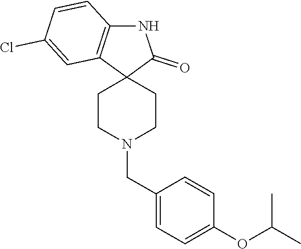 [SMILES: CC(C)Oc1ccc(CN2CCC3(CC2)C(=O)Nc2ccc(Cl)cc23)cc1]

8

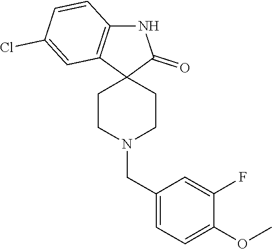 [SMILES: COc1ccc(CN2CCC3(CC2)C(=O)Nc2ccc(Cl)cc23)cc1F]

9

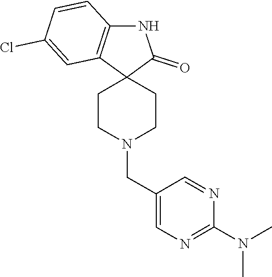 [SMILES: CN(C)c1ncc(CN2CCC3(CC2)C(=O)Nc2ccc(Cl)cc23)cn1]

10

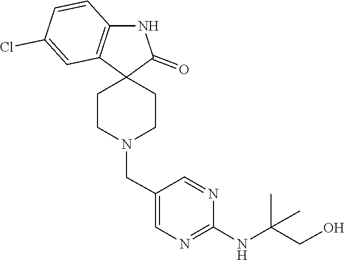 [SMILES: CC(C)(CO)Nc1ncc(CN2CCC3(CC2)C(=O)Nc2ccc(Cl)cc23)cn1]

11

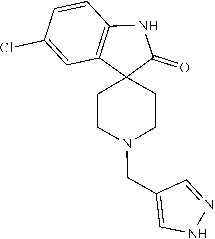 [SMILES: O=C1Nc2ccc(Cl)cc2C12CCN(Cc1cn[nH]c1)CC2]

12

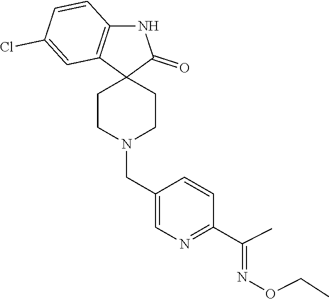 [SMILES: CCO/N=C(\C)c1ccc(CN2CCC3(CC2)C(=O)Nc2ccc(Cl)cc23)cn1]

13

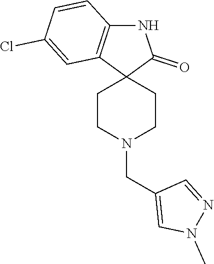 [SMILES: Cn1cc(CN2CCC3(CC2)C(=O)Nc2ccc(Cl)cc23)cn1]

14

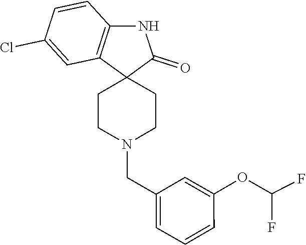 [SMILES: O=C1Nc2ccc(Cl)cc2C12CCN(Cc1cccc(OC(F)F)c1)CC2]

15

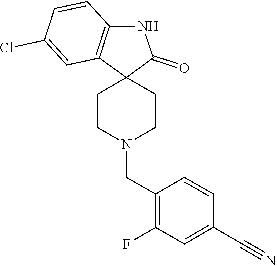 [SMILES: N#Cc1ccc(CN2CCC3(CC2)C(=O)Nc2ccc(Cl)cc23)c(F)c1]

16

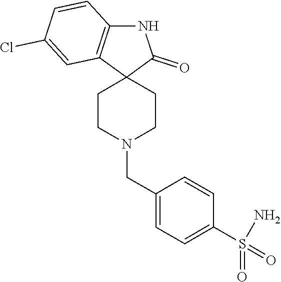 [SMILES: NS(=O)(=O)c1ccc(CN2CCC3(CC2)C(=O)Nc2ccc(Cl)cc23)cc1]

17

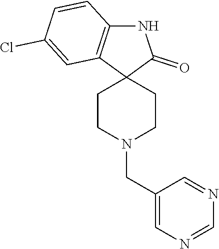 [SMILES: O=C1Nc2ccc(Cl)cc2C12CCN(Cc1cncnc1)CC2]

18

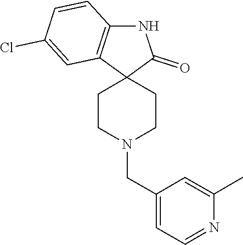 [SMILES: Cc1cc(CN2CCC3(CC2)C(=O)Nc2ccc(Cl)cc23)ccn1]

19

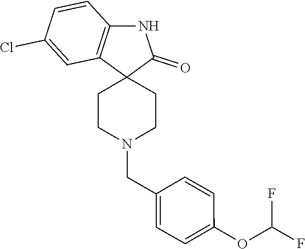 [SMILES: O=C1Nc2ccc(Cl)cc2C12CCN(Cc1ccc(OC(F)F)cc1)CC2]

20

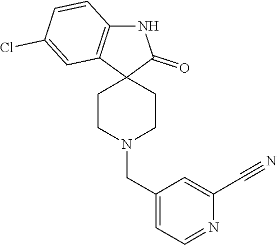 [SMILES: N#Cc1cc(CN2CCC3(CC2)C(=O)Nc2ccc(Cl)cc23)ccn1]

21

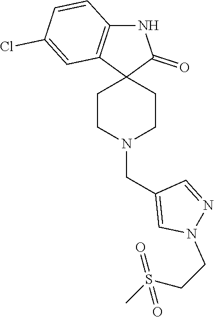 [SMILES: CS(=O)(=O)CCn1cc(CN2CCC3(CC2)C(=O)Nc2ccc(Cl)cc23)cn1]

22

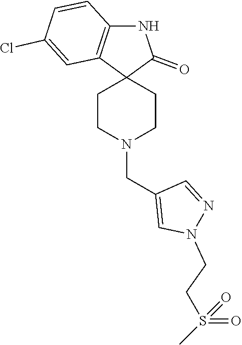 [SMILES: CS(=O)(=O)CCn1cc(CN2CCC3(CC2)C(=O)Nc2ccc(Cl)cc23)cn1]

23

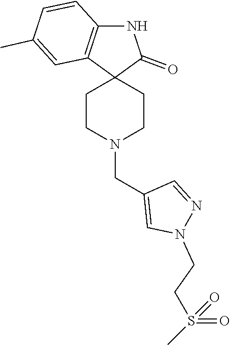 [SMILES: Cc1ccc2c(c1)C1(CCN(Cc3cnn(CCS(C)(=O)=O)c3)CC1)C(=O)N2]

24

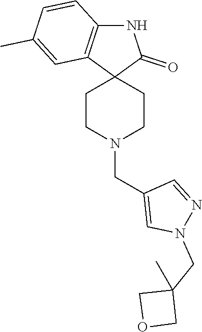 [SMILES: Cc1ccc2c(c1)C1(CCN(Cc3cnn(CC4(C)COC4)c3)CC1)C(=O)N2]

25

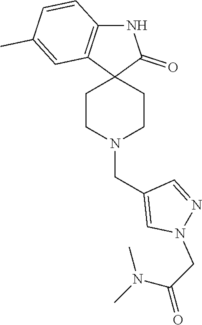 [SMILES: Cc1ccc2c(c1)C1(CCN(Cc3cnn(CC(=O)N(C)C)c3)CC1)C(=O)N2]

26

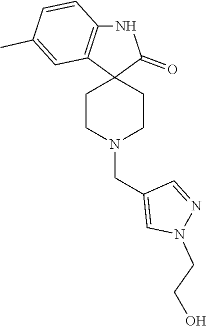 [SMILES: Cc1ccc2c(c1)C1(CCN(Cc3cnn(CCO)c3)CC1)C(=O)N2]

27

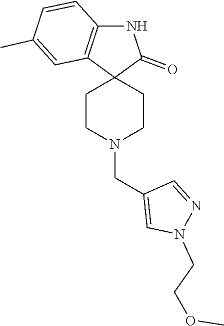

28

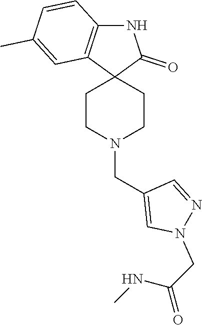 [SMILES: CNC(=O)Cn1cc(CN2CCC3(CC2)C(=O)Nc2ccc(C)cc23)cn1]

29

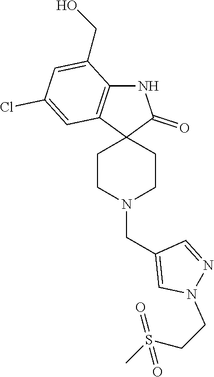 [SMILES: CS(=O)(=O)CCn1cc(CN2CCC3(CC2)C(=O)Nc2c(CO)cc(Cl)cc23)cn1]

30

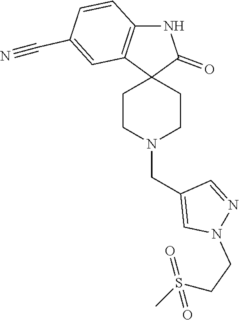 [SMILES: CS(=O)(=O)CCn1cc(CN2CCC3(CC2)C(=O)Nc2ccc(C#N)cc23)cn1]

31

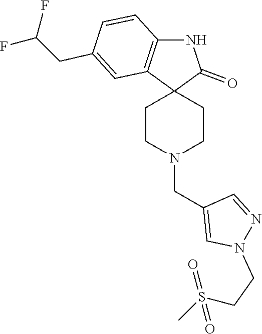 [SMILES: CS(=O)(=O)CCn1cc(CN2CCC3(CC2)C(=O)Nc2ccc(CC(F)F)cc23)cn1]

32

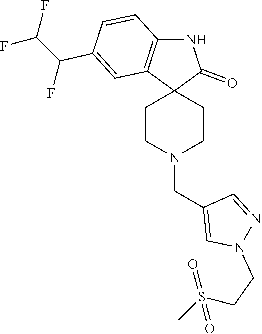 [SMILES: CS(=O)(=O)CCn1cc(CN2CCC3(CC2)C(=O)Nc2ccc(C(F)C(F)F)cc23)cn1]

33

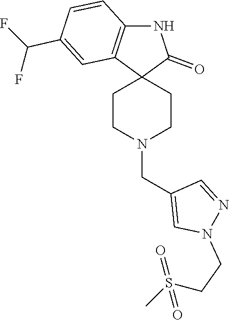 [SMILES: CS(=O)(=O)CCn1cc(CN2CCC3(CC2)C(=O)Nc2ccc(C(F)F)cc23)cn1]

34

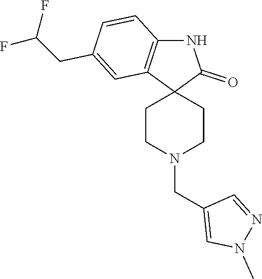 [SMILES: Cn1cc(CN2CCC3(CC2)C(=O)Nc2ccc(CC(F)F)cc23)cn1]

35

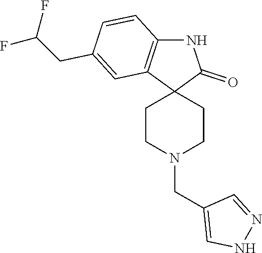 [SMILES: O=C1Nc2ccc(CC(F)F)cc2C12CCN(Cc1cn[nH]c1)CC2]

36

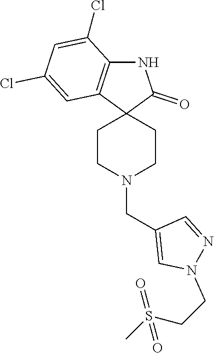 [SMILES: CS(=O)(=O)CCn1cc(CN2CCC3(CC2)C(=O)Nc2c(Cl)cc(Cl)cc23)cn1]

37

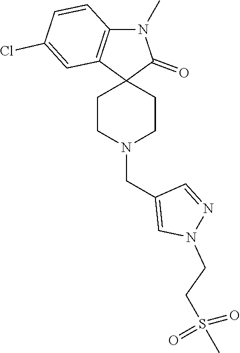 [SMILES: CN1C(=O)C2(CCN(Cc3cnn(CCS(C)(=O)=O)c3)CC2)c2cc(Cl)ccc21]

38

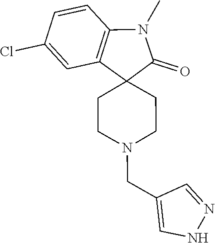 [SMILES: CN1C(=O)C2(CCN(Cc3cn[nH]c3)CC2)c2cc(Cl)ccc21]

39

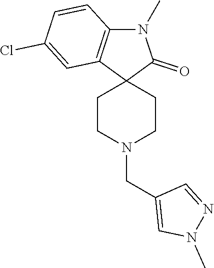 [SMILES: CN1C(=O)C2(CCN(Cc3cnn(C)c3)CC2)c2cc(Cl)ccc21]

40

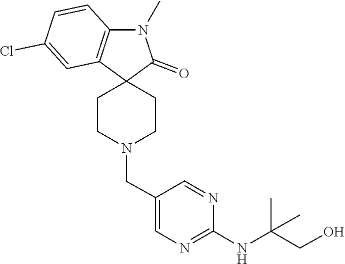 [SMILES: CN1C(=O)C2(CCN(Cc3cnc(NC(C)(C)CO)nc3)CC2)c2cc(Cl)ccc21]

41

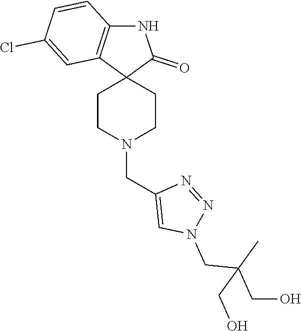 [SMILES: CC(CO)(CO)Cn1cc(CN2CCC3(CC2)C(=O)Nc2ccc(Cl)cc23)nn1]

42

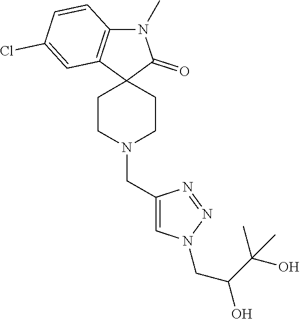 [SMILES: CN1C(=O)C2(CCN(Cc3cn(CC(O)C(C)(C)O)nn3)CC2)c2cc(Cl)ccc21]

43

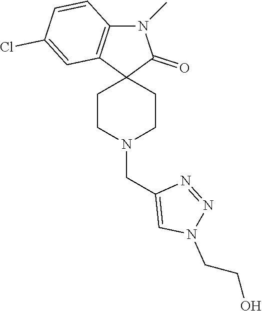 [SMILES: CN1C(=O)C2(CCN(Cc3cn(CCO)nn3)CC2)c2cc(Cl)ccc21]

44

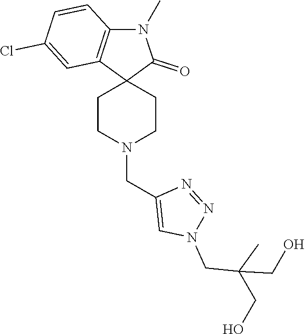 [SMILES: CN1C(=O)C2(CCN(Cc3cn(CC(C)(CO)CO)nn3)CC2)c2cc(Cl)ccc21]

45

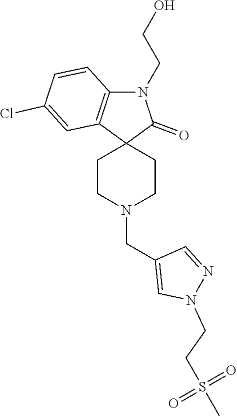 [SMILES: CS(=O)(=O)CCn1cc(CN2CCC3(CC2)C(=O)N(CCO)c2ccc(Cl)cc23)cn1]

46

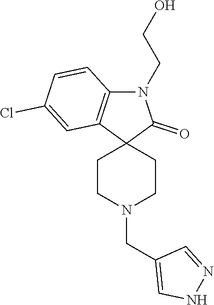 [SMILES: O=C1N(CCO)c2ccc(Cl)cc2C12CCN(Cc1cn[nH]c1)CC2]

47

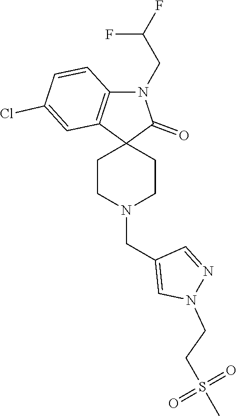 [SMILES: CS(=O)(=O)CCn1cc(CN2CCC3(CC2)C(=O)N(CC(F)F)c2ccc(Cl)cc23)cn1]

48

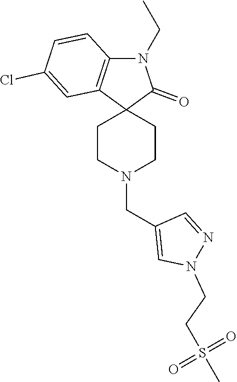 [SMILES: CCN1C(=O)C2(CCN(Cc3cnn(CCS(C)(=O)=O)c3)CC2)c2cc(Cl)ccc21]

49

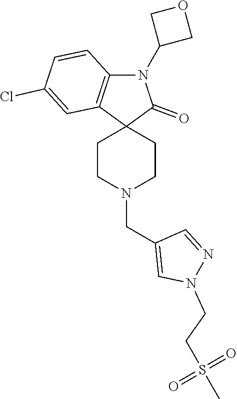 [SMILES: CS(=O)(=O)CCn1cc(CN2CCC3(CC2)C(=O)N(C2COC2)c2ccc(Cl)cc23)cn1]

50

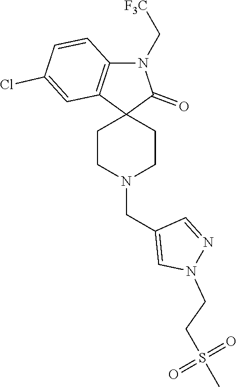 [SMILES: CS(=O)(=O)CCn1cc(CN2CCC3(CC2)C(=O)N(CC(F)(F)F)c2ccc(Cl)cc23)cn1]

51

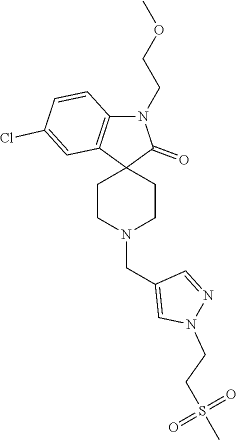 [SMILES: COCCN1C(=O)C2(CCN(Cc3cnn(CCS(C)(=O)=O)c3)CC2)c2cc(Cl)ccc21]

52

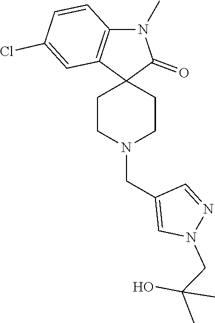 [SMILES: CN1C(=O)C2(CCN(Cc3cnn(CC(C)(C)O)c3)CC2)c2cc(Cl)ccc21]

53

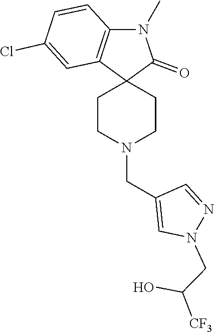 [SMILES: CN1C(=O)C2(CCN(Cc3cnn(CC(O)C(F)(F)F)c3)CC2)c2cc(Cl)ccc21]

54

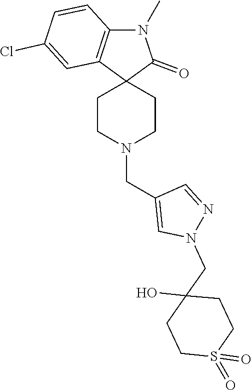 [SMILES: CN1C(=O)C2(CCN(Cc3cnn(CC4(O)CCS(=O)(=O)CC4)c3)CC2)c2cc(Cl)ccc21]

55

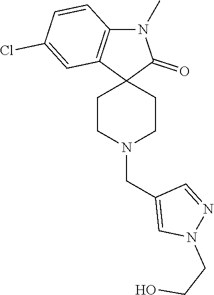 [SMILES: CN1C(=O)C2(CCN(Cc3cnn(CCO)c3)CC2)c2cc(Cl)ccc21]

56

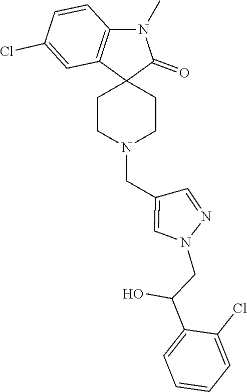 [SMILES: CN1C(=O)C2(CCN(Cc3cnn(CC(O)c4ccccc4Cl)c3)CC2)c2cc(Cl)ccc21]

57

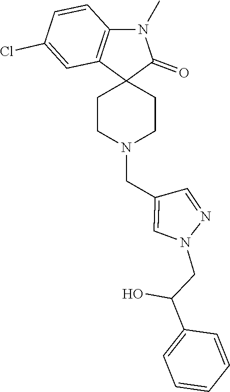 [SMILES: CN1C(=O)C2(CCN(Cc3cnn(CC(O)c4ccccc4)c3)CC2)c2cc(Cl)ccc21]

58

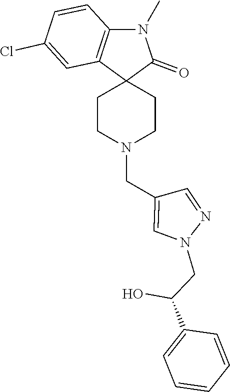 [SMILES: CN1C(=O)C2(CCN(Cc3cnn(C[C@@H](O)c4ccccc4)c3)CC2)c2cc(Cl)ccc21]

59

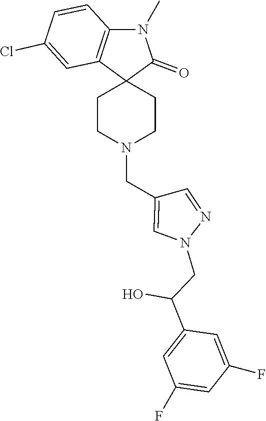 [SMILES: CN1C(=O)C2(CCN(Cc3cnn(CC(O)c4cc(F)cc(F)c4)c3)CC2)c2cc(Cl)ccc21]

60

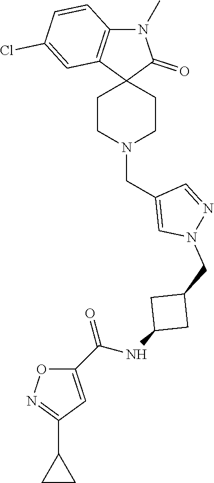 [SMILES: CN1C(=O)C2(CCN(Cc3cnn(C[C@H]4C[C@@H](NC(=O)c5cc(C6CC6)no5)C4)c3)CC2)c2cc(Cl)ccc21]

61

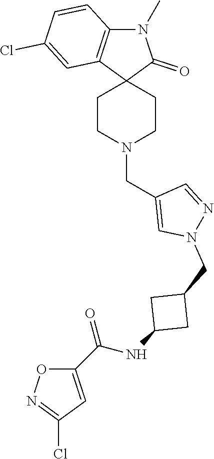 [SMILES: CN1C(=O)C2(CCN(Cc3cnn(C[C@H]4C[C@@H](NC(=O)c5cc(Cl)no5)C4)c3)CC2)c2cc(Cl)ccc21]

62

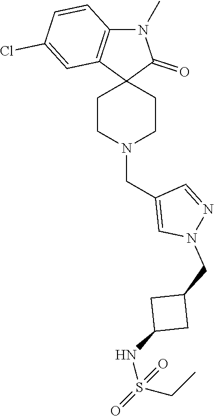 [SMILES: CCS(=O)(=O)N[C@H]1C[C@@H](Cn2cc(CN3CCC4(CC3)C(=O)N(C)c3ccc(Cl)cc34)cn2)C1]

63

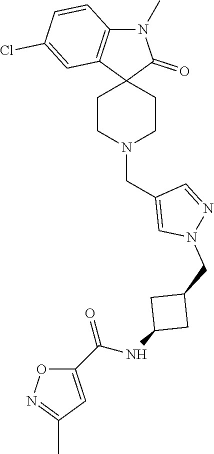 [SMILES: Cc1cc(C(=O)N[C@H]2C[C@@H](Cn3cc(CN4CCC5(CC4)C(=O)N(C)c4ccc(Cl)cc45)cn3)C2)on1]

64

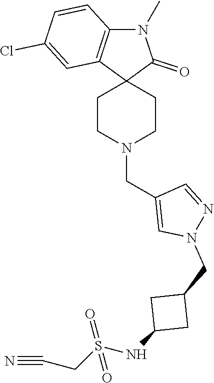 [SMILES: CN1C(=O)C2(CCN(Cc3cnn(C[C@H]4C[C@@H](NS(=O)(=O)CC#N)C4)c3)CC2)c2cc(Cl)ccc21]

65

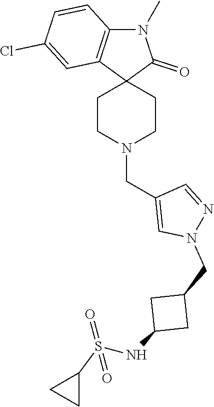 [SMILES: CN1C(=O)C2(CCN(Cc3cnn(C[C@H]4C[C@@H](NS(=O)(=O)C5CC5)C4)c3)CC2)c2cc(Cl)ccc21]

66

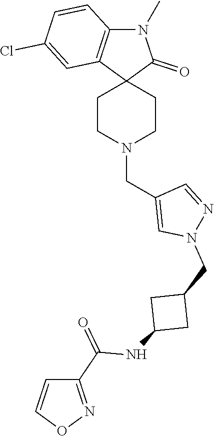 [SMILES: CN1C(=O)C2(CCN(Cc3cnn(C[C@H]4C[C@@H](NC(=O)c5ccon5)C4)c3)CC2)c2cc(Cl)ccc21]

67

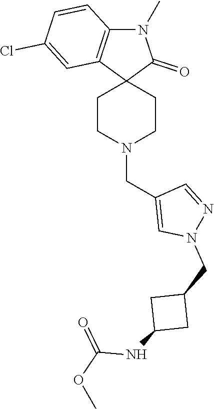 [SMILES: COC(=O)N[C@H]1C[C@@H](Cn2cc(CN3CCC4(CC3)C(=O)N(C)c3ccc(Cl)cc34)cn2)C1]

68

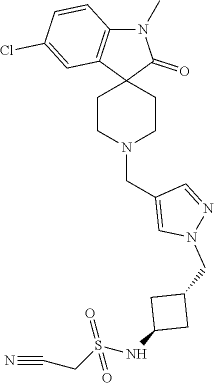 [SMILES: CN1C(=O)C2(CCN(Cc3cnn(C[C@H]4C[C@H](NS(=O)(=O)CC#N)C4)c3)CC2)c2cc(Cl)ccc21]

69

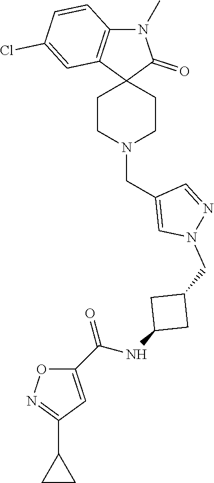 [SMILES: CN1C(=O)C2(CCN(Cc3cnn(C[C@H]4C[C@H](NC(=O)c5cc(C6CC6)no5)C4)c3)CC2)c2cc(Cl)ccc21]

70

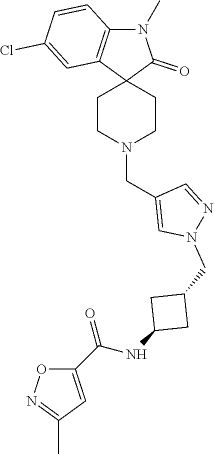 [SMILES: Cc1cc(C(=O)N[C@H]2C[C@H](Cn3cc(CN4CCC5(CC4)C(=O)N(C)c4ccc(Cl)cc45)cn3)C2)on1]

71

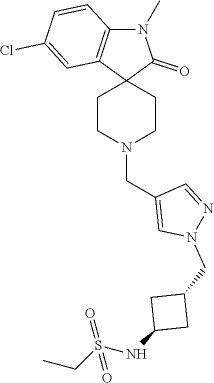 [SMILES: CCS(=O)(=O)N[C@H]1C[C@H](Cn2cc(CN3CCC4(CC3)C(=O)N(C)c3ccc(Cl)cc34)cn2)C1]

72

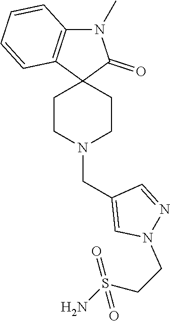 [SMILES: CN1C(=O)C2(CCN(Cc3cnn(CCS(N)(=O)=O)c3)CC2)c2ccccc21]

73

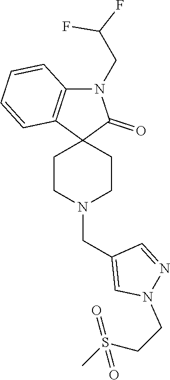 [SMILES: CS(=O)(=O)CCn1cc(CN2CCC3(CC2)C(=O)N(CC(F)F)c2ccccc23)cn1]

74

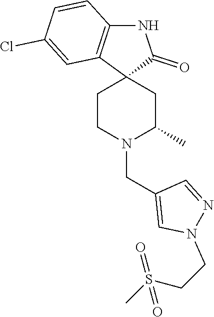 [SMILES: C[C@H]1C[C@]2(CCN1Cc1cnn(CCS(C)(=O)=O)c1)C(=O)Nc1ccc(Cl)cc12]

75

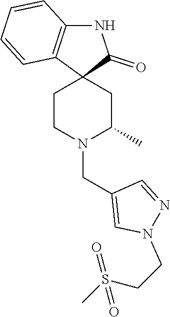 [SMILES: C[C@H]1C[C@@]2(CCN1Cc1cnn(CCS(C)(=O)=O)c1)C(=O)Nc1ccccc12]

76

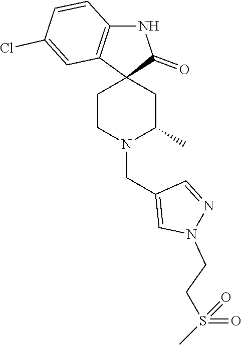 [SMILES: C[C@H]1C[C@@]2(CCN1Cc1cnn(CCS(C)(=O)=O)c1)C(=O)Nc1ccc(Cl)cc12]

77

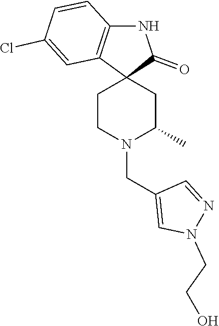 [SMILES: C[C@H]1C[C@@]2(CCN1Cc1cnn(CCO)c1)C(=O)Nc1ccc(Cl)cc12]

78

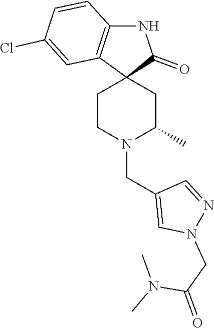 [SMILES: C[C@H]1C[C@@]2(CCN1Cc1cnn(CC(=O)N(C)C)c1)C(=O)Nc1ccc(Cl)cc12]

79

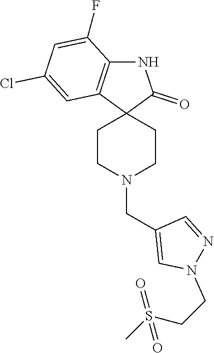 [SMILES: CS(=O)(=O)CCn1cc(CN2CCC3(CC2)C(=O)Nc2c(F)cc(Cl)cc23)cn1]

80

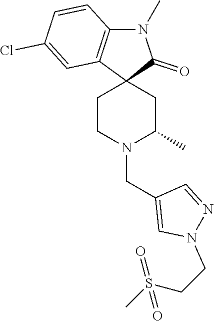 [SMILES: C[C@H]1C[C@@]2(CCN1Cc1cnn(CCS(C)(=O)=O)c1)C(=O)N(C)c1ccc(Cl)cc12]

81

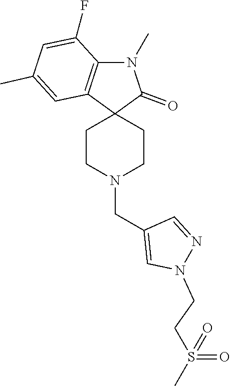 [SMILES: Cc1cc(F)c2c(c1)C1(CCN(Cc3cnn(CCS(C)(=O)=O)c3)CC1)C(=O)N2C]

82

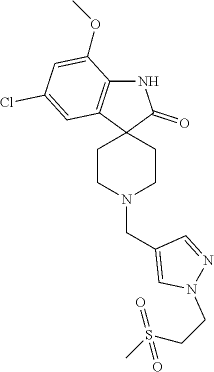 [SMILES: COc1cc(Cl)cc2c1NC(=O)C21CCN(Cc2cnn(CCS(C)(=O)=O)c2)CC1]

83

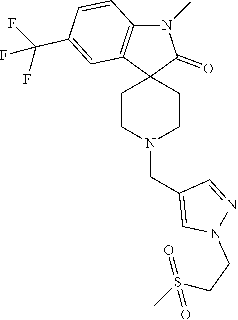 [SMILES: CN1C(=O)C2(CCN(Cc3cnn(CCS(C)(=O)=O)c3)CC2)c2cc(C(F)(F)F)ccc21]

84

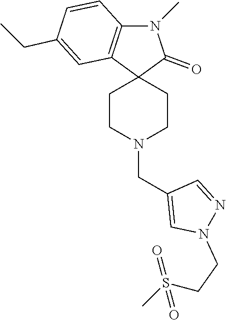 [SMILES: CCc1ccc2c(c1)C1(CCN(Cc3cnn(CCS(C)(=O)=O)c3)CC1)C(=O)N2C]

85

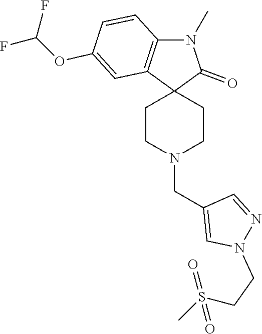 [SMILES: CN1C(=O)C2(CCN(Cc3cnn(CCS(C)(=O)=O)c3)CC2)c2cc(OC(F)F)ccc21]

86

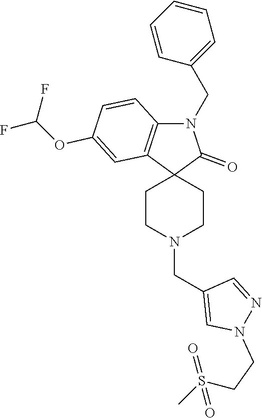 [SMILES: CS(=O)(=O)CCn1cc(CN2CCC3(CC2)C(=O)N(Cc2ccccc2)c2ccc(OC(F)F)cc23)cn1]

87

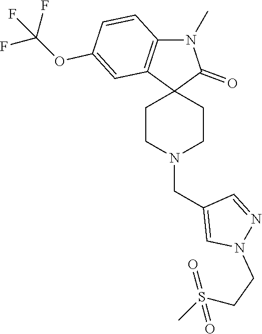 [SMILES: CN1C(=O)C2(CCN(Cc3cnn(CCS(C)(=O)=O)c3)CC2)c2cc(OC(F)(F)F)ccc21]

88

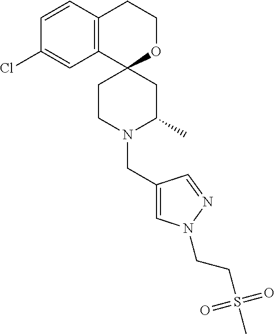 [SMILES: C[C@H]1C[C@@]2(CCN1Cc1cnn(CCS(C)(=O)=O)c1)OCCc1ccc(Cl)cc12]

89

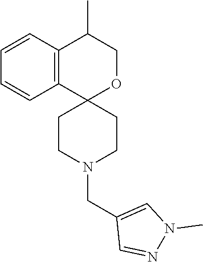 [SMILES: CC1COC2(CCN(Cc3cnn(C)c3)CC2)c2ccccc21]

90

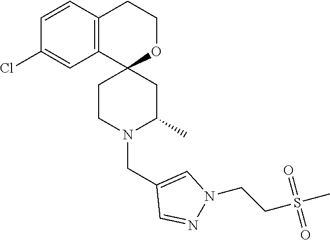 [SMILES: C[C@H]1C[C@@]2(CCN1Cc1cnn(CCS(C)(=O)=O)c1)OCCc1ccc(Cl)cc12]

91

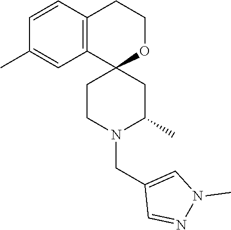

92

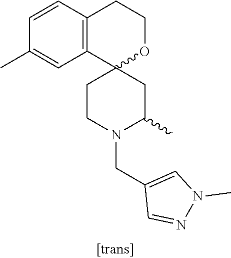

93

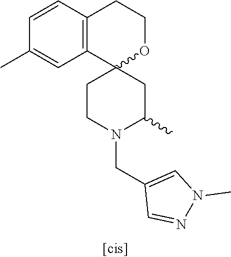

94

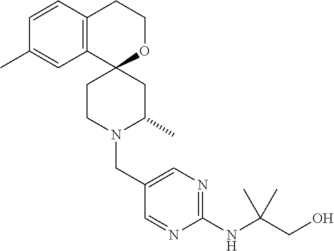 [SMILES: Cc1ccc2c(c1)[C@]1(CCN(Cc3cnc(NC(C)(C)CO)nc3)[C@@H](C)C1)OCC2]

95

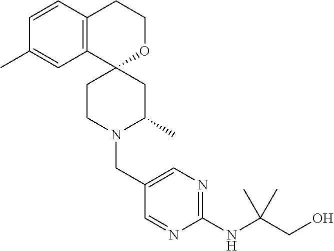 [SMILES: Cc1ccc2c(c1)[C@@]1(CCN(Cc3cnc(NC(C)(C)CO)nc3)[C@@H](C)C1)OCC2]

96

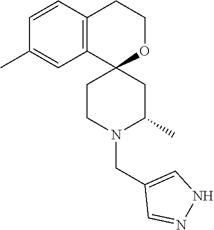 [SMILES: Cc1ccc2c(c1)[C@]1(CCN(Cc3cn[nH]c3)[C@@H](C)C1)OCC2]

97

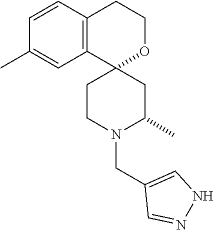

98

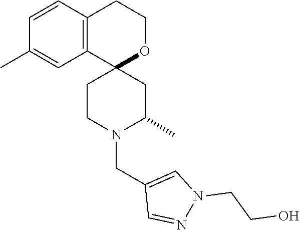

99

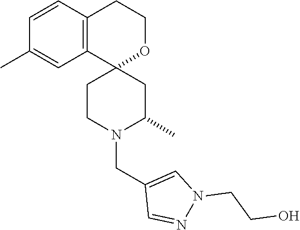

100

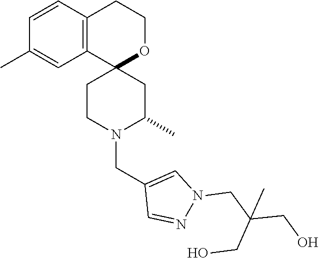 [SMILES: Cc1ccc2c(c1)[C@]1(CCN(Cc3cnn(CC(C)(CO)CO)c3)[C@@H](C)C1)OCC2]

101

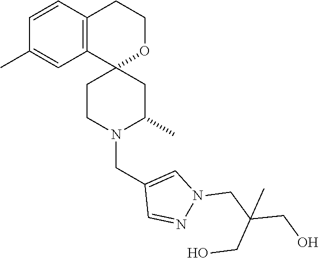 [SMILES: Cc1ccc2c(c1)[C@@]1(CCN(Cc3cnn(CC(C)(CO)CO)c3)[C@@H](C)C1)OCC2]

102

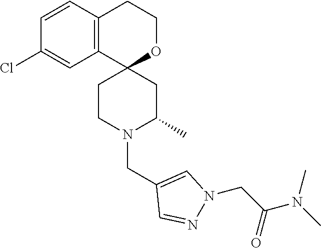 [SMILES: C[C@H]1C[C@@]2(CCN1Cc1cnn(CC(=O)N(C)C)c1)OCCc1ccc(Cl)cc12]

103

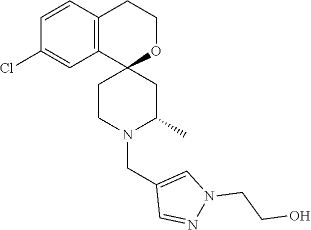 [SMILES: C[C@H]1C[C@@]2(CCN1Cc1cnn(CCO)c1)OCCc1ccc(Cl)cc12]

104

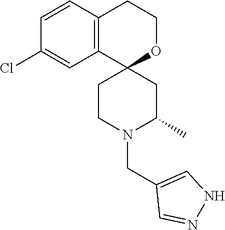 [SMILES: C[C@H]1C[C@@]2(CCN1Cc1cn[nH]c1)OCCc1ccc(Cl)cc12]

105

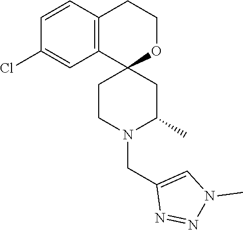 [SMILES: C[C@H]1C[C@@]2(CCN1Cc1cn(C)nn1)OCCc1ccc(Cl)cc12]

106

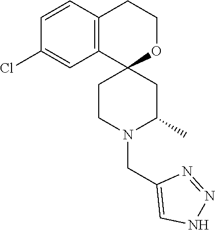 [SMILES: C[C@H]1C[C@@]2(CCN1Cc1c[nH]nn1)OCCc1ccc(Cl)cc12]

107

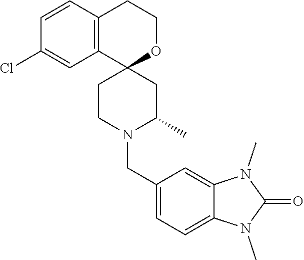 [SMILES: C[C@H]1C[C@@]2(CCN1Cc1ccc3c(c1)n(C)c(=O)n3C)OCCc1ccc(Cl)cc12]

108

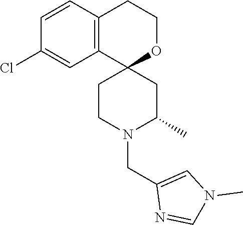 [SMILES: C[C@H]1C[C@@]2(CCN1Cc1cn(C)cn1)OCCc1ccc(Cl)cc12]

109

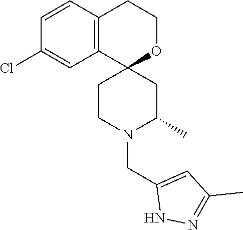 [SMILES: Cc1cc(CN2CC[C@]3(C[C@@H]2C)OCCc2ccc(Cl)cc23)[nH]n1]

110

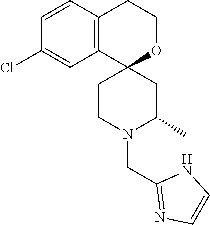 [SMILES: C[C@H]1C[C@@]2(CCN1Cc1ncc[nH]1)OCCc1ccc(Cl)cc12]

111

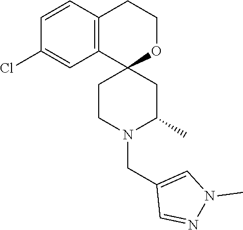 [SMILES: C[C@H]1C[C@@]2(CCN1Cc1cnn(C)c1)OCCc1ccc(Cl)cc12]

112

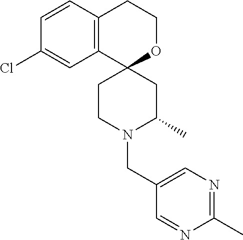 [SMILES: Cc1ncc(CN2CC[C@]3(C[C@@H]2C)OCCc2ccc(Cl)cc23)cn1]

113

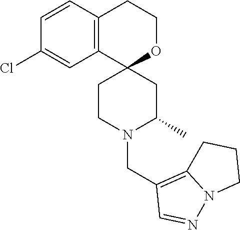 [SMILES: C[C@H]1C[C@@]2(CCN1Cc1cnn3c1CCC3)OCCc1ccc(Cl)cc12]

114

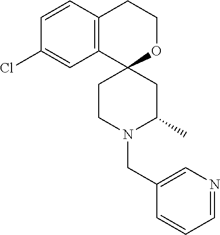 [SMILES: C[C@H]1C[C@@]2(CCN1Cc1cccnc1)OCCc1ccc(Cl)cc12]

115

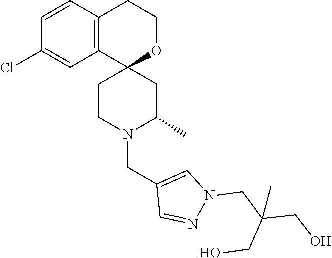 [SMILES: C[C@H]1C[C@@]2(CCN1Cc1cnn(CC(C)(CO)CO)c1)OCCc1ccc(Cl)cc12]

116

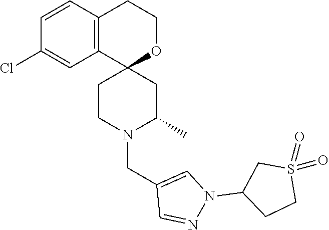 [SMILES: C[C@H]1C[C@@]2(CCN1Cc1cnn(C3CCS(=O)(=O)C3)c1)OCCc1ccc(Cl)cc12]

117

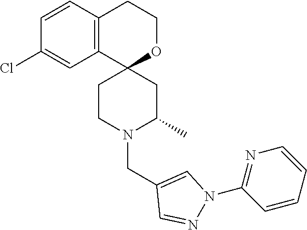 [SMILES: C[C@H]1C[C@@]2(CCN1Cc1cnn(-c3ccccn3)c1)OCCc1ccc(Cl)cc12]

118

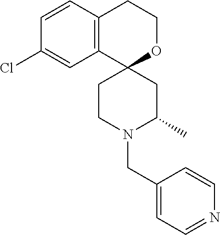 [SMILES: C[C@H]1C[C@@]2(CCN1Cc1ccncc1)OCCc1ccc(Cl)cc12]

119

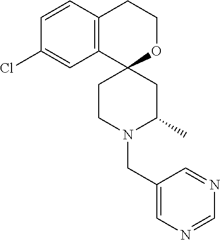 [SMILES: C[C@H]1C[C@@]2(CCN1Cc1cncnc1)OCCc1ccc(Cl)cc12]

120

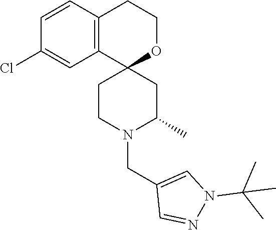 [SMILES: C[C@H]1C[C@@]2(CCN1Cc1cnn(C(C)(C)C)c1)OCCc1ccc(Cl)cc12]

121

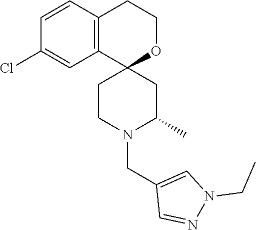 [SMILES: CCn1cc(CN2CC[C@]3(C[C@@H]2C)OCCc2ccc(Cl)cc23)cn1]

122

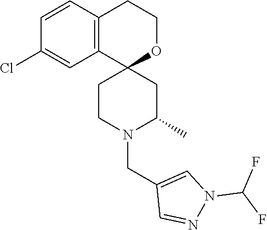 [SMILES: C[C@H]1C[C@@]2(CCN1Cc1cnn(C(F)F)c1)OCCc1ccc(Cl)cc12]

123

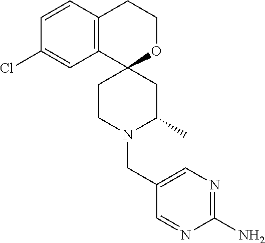 [SMILES: C[C@H]1C[C@@]2(CCN1Cc1cnc(N)nc1)OCCc1ccc(Cl)cc12]

124

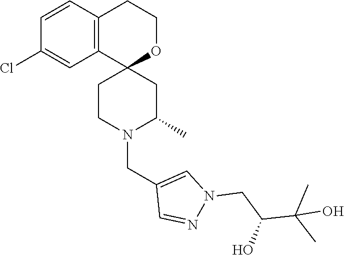 [SMILES: C[C@H]1C[C@@]2(CCN1Cc1cnn(C[C@@H](O)C(C)(C)O)c1)OCCc1ccc(Cl)cc12]

125

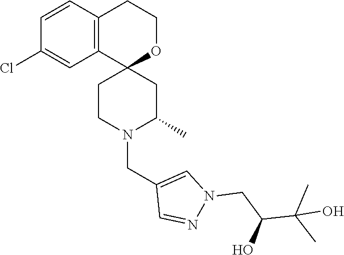 [SMILES: C[C@H]1C[C@@]2(CCN1Cc1cnn(C[C@H](O)C(C)(C)O)c1)OCCc1ccc(Cl)cc12]

126

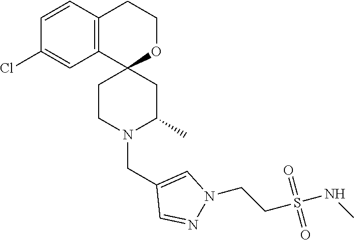 [SMILES: CNS(=O)(=O)CCn1cc(CN2CC[C@]3(C[C@@H]2C)OCCc2ccc(Cl)cc23)cn1]

127

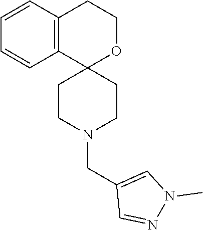 [SMILES: Cn1cc(CN2CCC3(CC2)OCCc2ccccc23)cn1]

128

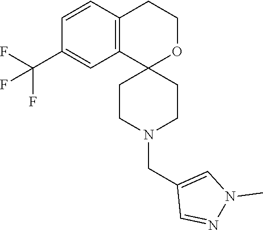 [SMILES: Cn1cc(CN2CCC3(CC2)OCCc2ccc(C(F)(F)F)cc23)cn1]

129

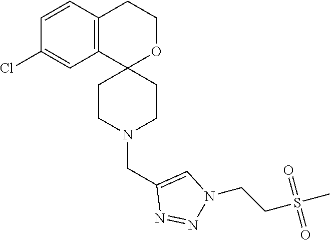 [SMILES: CS(=O)(=O)CCn1cc(CN2CCC3(CC2)OCCc2ccc(Cl)cc23)nn1]

130

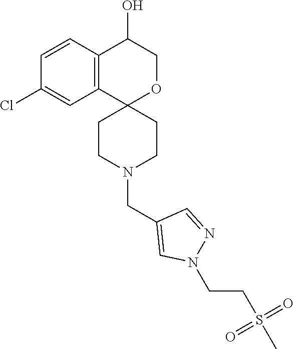 [SMILES: CS(=O)(=O)CCn1cc(CN2CCC3(CC2)OCC(O)c2ccc(Cl)cc23)cn1]

131

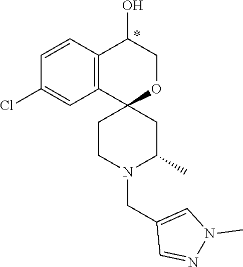

132

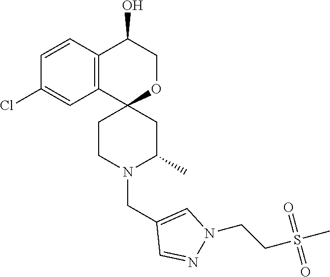 [SMILES: C[C@H]1C[C@@]2(CCN1Cc1cnn(CCS(C)(=O)=O)c1)OC[C@H](O)c1ccc(Cl)cc12]

133

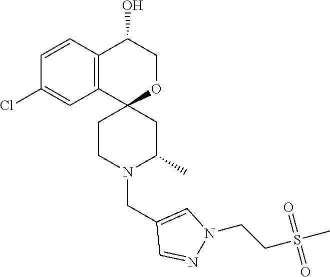 [SMILES: C[C@H]1C[C@@]2(CCN1Cc1cnn(CCS(C)(=O)=O)c1)OC[C@@H](O)c1ccc(Cl)cc12]

134

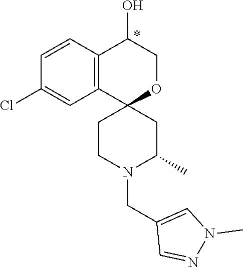

135

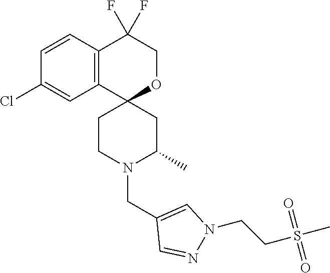 [SMILES: C[C@H]1C[C@@]2(CCN1Cc1cnn(CCS(C)(=O)=O)c1)OCC(F)(F)c1ccc(Cl)cc12]

136

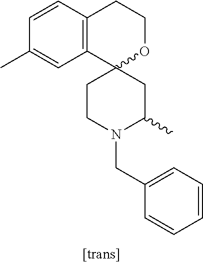 [SMILES: Cc1ccc2c(c1)C1(CCN(Cc3ccccc3)C(C)C1)OCC2]

137

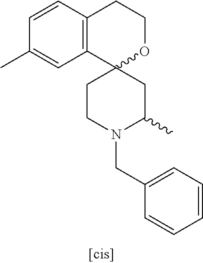 [SMILES: Cc1ccc2c(c1)C1(CCN(Cc3ccccc3)C(C)C1)OCC2]

138

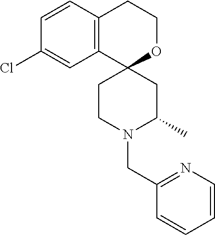 [SMILES: C[C@H]1C[C@@]2(CCN1Cc1ccccn1)OCCc1ccc(Cl)cc12]

139

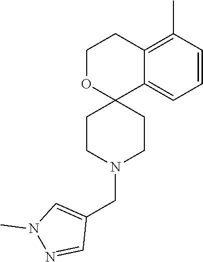 [SMILES: Cc1cccc2c1CCOC21CCN(Cc2cnn(C)c2)CC1]

140

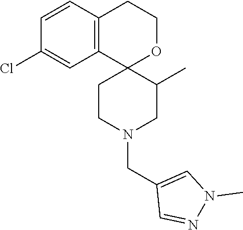 [SMILES: CC1CN(Cc2cnn(C)c2)CCC12OCCc1ccc(Cl)cc12]

141

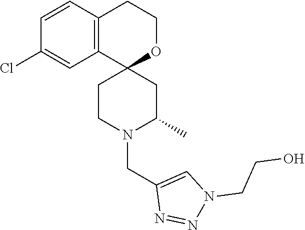 [SMILES: C[C@H]1C[C@@]2(CCN1Cc1cn(CCO)nn1)OCCc1ccc(Cl)cc12]

142

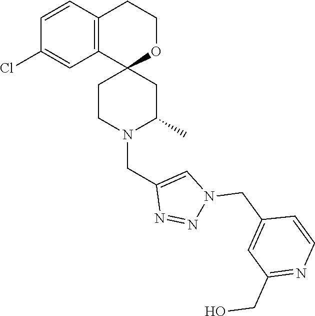 [SMILES: C[C@H]1C[C@@]2(CCN1Cc1cn(Cc3ccnc(CO)c3)nn1)OCCc1ccc(Cl)cc12]

143

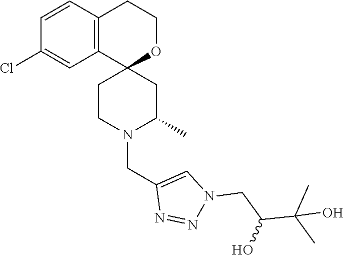 [SMILES: C[C@H]1C[C@@]2(CCN1Cc1cn(CC(O)C(C)(C)O)nn1)OCCc1ccc(Cl)cc12]

144

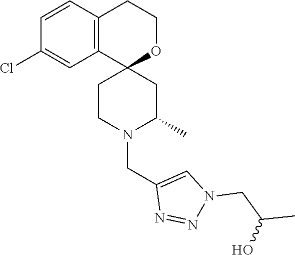 [SMILES: CC(O)Cn1cc(CN2CC[C@]3(C[C@@H]2C)OCCc2ccc(Cl)cc23)nn1]

145

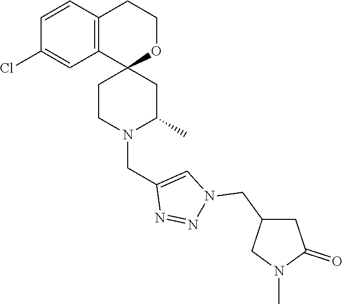 [SMILES: C[C@H]1C[C@@]2(CCN1Cc1cn(CC3CC(=O)N(C)C3)nn1)OCCc1ccc(Cl)cc12]

146

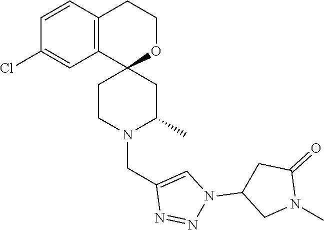 [SMILES: C[C@H]1C[C@@]2(CCN1Cc1cn(C3CC(=O)N(C)C3)nn1)OCCc1ccc(Cl)cc12]

147

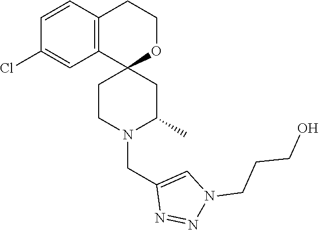 [SMILES: C[C@H]1C[C@@]2(CCN1Cc1cn(CCCO)nn1)OCCc1ccc(Cl)cc12]

148

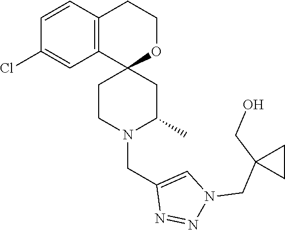 [SMILES: C[C@H]1C[C@@]2(CCN1Cc1cn(CC3(CO)CC3)nn1)OCCc1ccc(Cl)cc12]

149

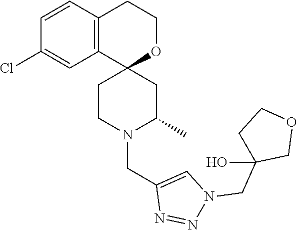 [SMILES: C[C@H]1C[C@@]2(CCN1Cc1cn(CC3(O)CCOC3)nn1)OCCc1ccc(Cl)cc12]

150

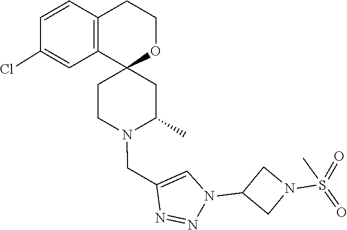 [SMILES: C[C@H]1C[C@@]2(CCN1Cc1cn(C3CN(S(C)(=O)=O)C3)nn1)OCCc1ccc(Cl)cc12]

151

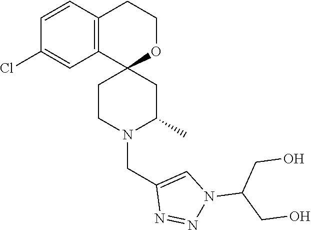 [SMILES: C[C@H]1C[C@@]2(CCN1Cc1cn(C(CO)CO)nn1)OCCc1ccc(Cl)cc12]

152

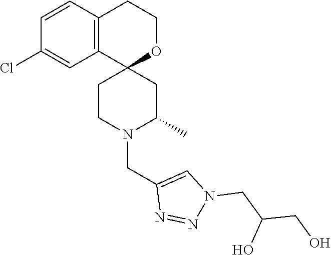 [SMILES: C[C@H]1C[C@@]2(CCN1Cc1cn(CC(O)CO)nn1)OCCc1ccc(Cl)cc12]

153

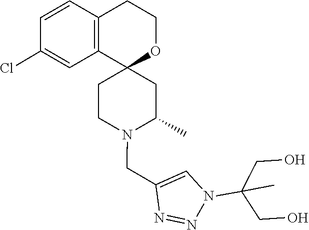 [SMILES: C[C@H]1C[C@@]2(CCN1Cc1cn(C(C)(CO)CO)nn1)OCCc1ccc(Cl)cc12]

154

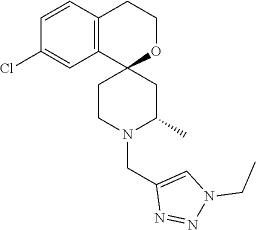 [SMILES: CCn1cc(CN2CC[C@]3(C[C@@H]2C)OCCc2ccc(Cl)cc23)nn1]

155

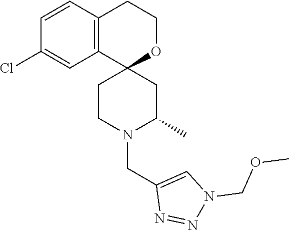 [SMILES: COCn1cc(CN2CC[C@]3(C[C@@H]2C)OCCc2ccc(Cl)cc23)nn1]

156

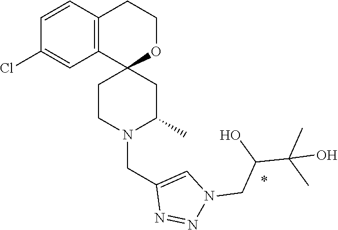 [SMILES: C[C@H]1C[C@@]2(CCN1Cc1cn(CC(O)C(C)(C)O)nn1)OCCc1ccc(Cl)cc12]

157

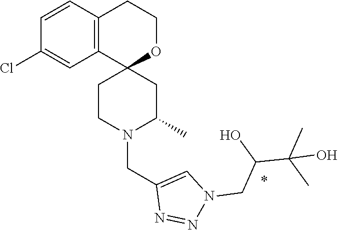 [SMILES: C[C@H]1C[C@@]2(CCN1Cc1cn(CC(O)C(C)(C)O)nn1)OCCc1ccc(Cl)cc12]

158

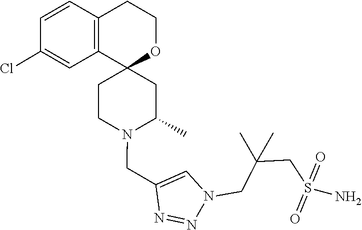 [SMILES: C[C@H]1C[C@@]2(CCN1Cc1cn(CC(C)(C)CS(N)(=O)=O)nn1)OCCc1ccc(Cl)cc12]

159

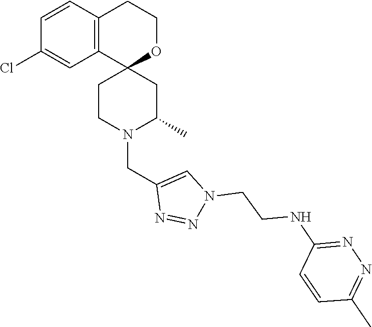 [SMILES: Cc1ccc(NCCn2cc(CN3CC[C@]4(C[C@@H]3C)OCCc3ccc(Cl)cc34)nn2)nn1]

160

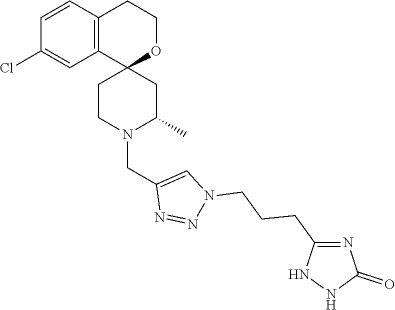 [SMILES: C[C@H]1C[C@@]2(CCN1Cc1cn(CCCc3nc(=O)[nH][nH]3)nn1)OCCc1ccc(Cl)cc12]

161

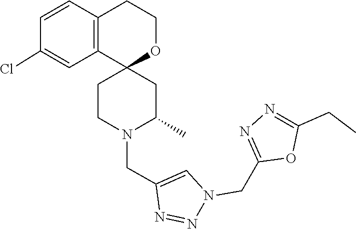 [SMILES: CCc1nnc(Cn2cc(CN3CC[C@]4(C[C@@H]3C)OCCc3ccc(Cl)cc34)nn2)o1]

162

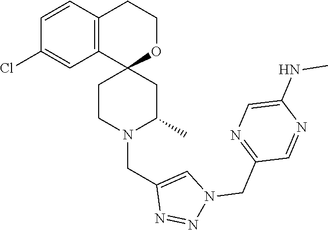 [SMILES: CNc1cnc(Cn2cc(CN3CC[C@]4(C[C@@H]3C)OCCc3ccc(Cl)cc34)nn2)cn1]

163

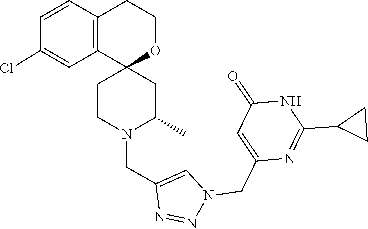 [SMILES: C[C@H]1C[C@@]2(CCN1Cc1cn(Cc3cc(=O)[nH]c(C4CC4)n3)nn1)OCCc1ccc(Cl)cc12]

164

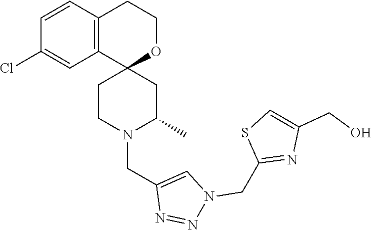 [SMILES: C[C@H]1C[C@@]2(CCN1Cc1cn(Cc3nc(CO)cs3)nn1)OCCc1ccc(Cl)cc12]

165

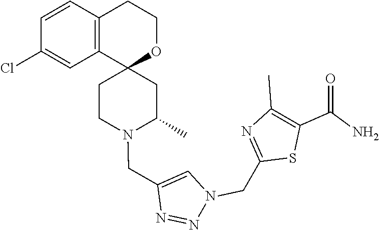 [SMILES: Cc1nc(Cn2cc(CN3CC[C@]4(C[C@@H]3C)OCCc3ccc(Cl)cc34)nn2)sc1C(N)=O]

166

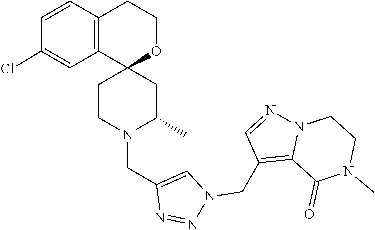 [SMILES: C[C@H]1C[C@@]2(CCN1Cc1cn(Cc3cnn4c3C(=O)N(C)CC4)nn1)OCCc1ccc(Cl)cc12]

167

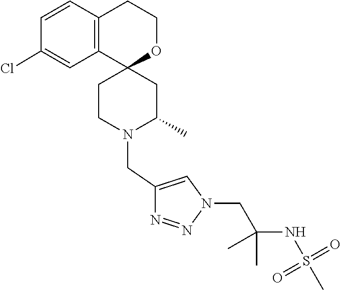 [SMILES: C[C@H]1C[C@@]2(CCN1Cc1cn(CC(C)(C)NS(C)(=O)=O)nn1)OCCc1ccc(Cl)cc12]

168

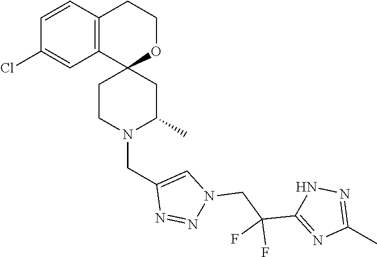 [SMILES: Cc1n[nH]c(C(F)(F)Cn2cc(CN3CC[C@]4(C[C@@H]3C)OCCc3ccc(Cl)cc34)nn2)n1]

169

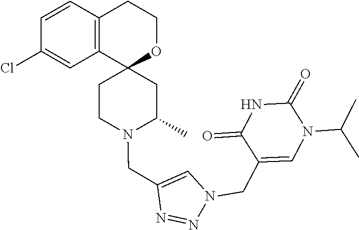 [SMILES: CC(C)n1cc(Cn2cc(CN3CC[C@]4(C[C@@H]3C)OCCc3ccc(Cl)cc34)nn2)c(=O)[nH]c1=O]

170

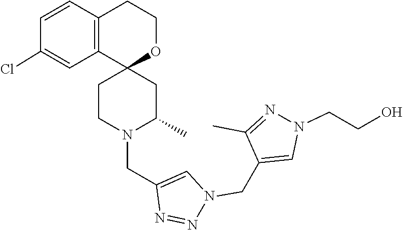 [SMILES: Cc1nn(CCO)cc1Cn1cc(CN2CC[C@]3(C[C@@H]2C)OCCc2ccc(Cl)cc23)nn1]

171

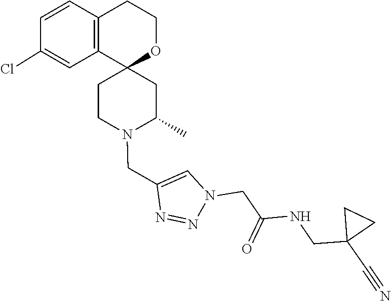 [SMILES: C[C@H]1C[C@@]2(CCN1Cc1cn(CC(=O)NCC3(C#N)CC3)nn1)OCCc1ccc(Cl)cc12]

172

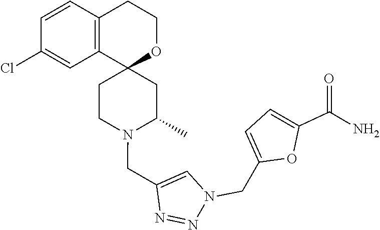 [SMILES: C[C@H]1C[C@@]2(CCN1Cc1cn(Cc3ccc(C(N)=O)o3)nn1)OCCc1ccc(Cl)cc12]

173

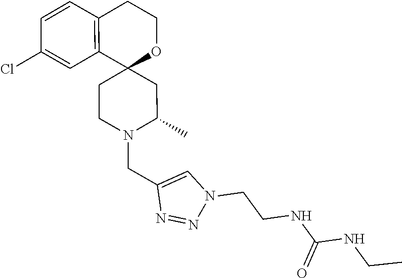 [SMILES: CCNC(=O)NCCn1cc(CN2CC[C@]3(C[C@@H]2C)OCCc2ccc(Cl)cc23)nn1]

174

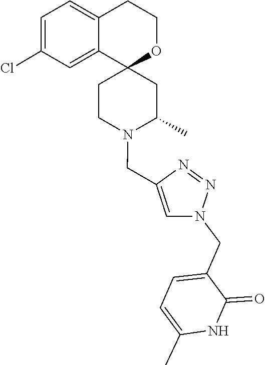 [SMILES: Cc1ccc(Cn2cc(CN3CC[C@]4(C[C@@H]3C)OCCc3ccc(Cl)cc34)nn2)c(=O)[nH]1]

175

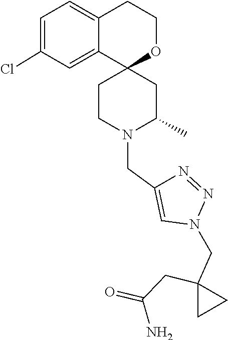 [SMILES: C[C@H]1C[C@@]2(CCN1Cc1cn(CC3(CC(N)=O)CC3)nn1)OCCc1ccc(Cl)cc12]

176

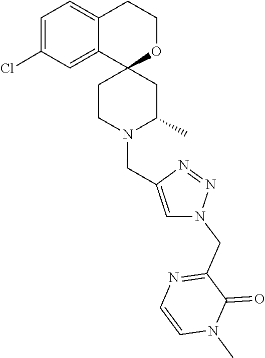 [SMILES: C[C@H]1C[C@@]2(CCN1Cc1cn(Cc3nccn(C)c3=O)nn1)OCCc1ccc(Cl)cc12]

177

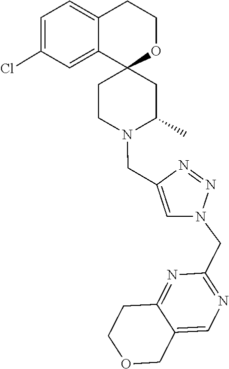 [SMILES: C[C@H]1C[C@@]2(CCN1Cc1cn(Cc3ncc4c(n3)CCOC4)nn1)OCCc1ccc(Cl)cc12]

178

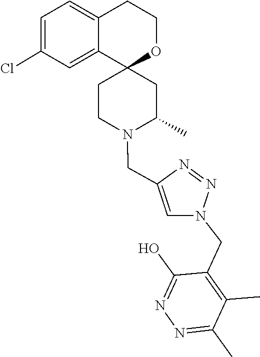 [SMILES: Cc1nnc(O)c(Cn2cc(CN3CC[C@]4(C[C@@H]3C)OCCc3ccc(Cl)cc34)nn2)c1C]

179

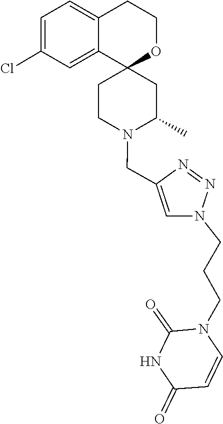 [SMILES: C[C@H]1C[C@@]2(CCN1Cc1cn(CCCn3ccc(=O)[nH]c3=O)nn1)OCCc1ccc(Cl)cc12]

180

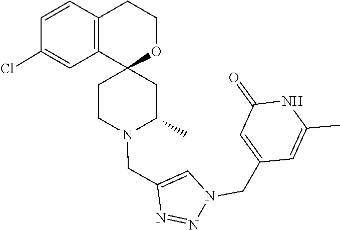 [SMILES: Cc1cc(Cn2cc(CN3CC[C@]4(C[C@@H]3C)OCCc3ccc(Cl)cc34)nn2)cc(=O)[nH]1]

181

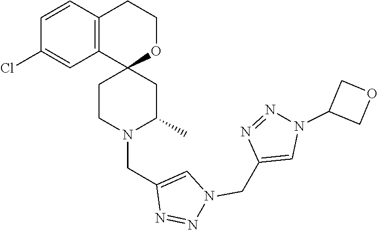 [SMILES: C[C@H]1C[C@@]2(CCN1Cc1cn(Cc3cn(C4COC4)nn3)nn1)OCCc1ccc(Cl)cc12]

182

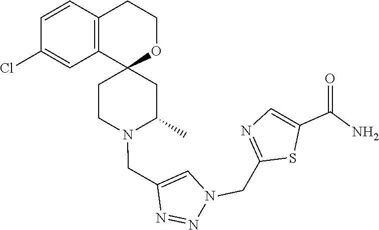 [SMILES: C[C@H]1C[C@@]2(CCN1Cc1cn(Cc3ncc(C(N)=O)s3)nn1)OCCc1ccc(Cl)cc12]

183

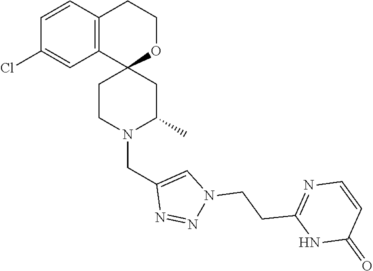 [SMILES: C[C@H]1C[C@@]2(CCN1Cc1cn(CCc3nccc(=O)[nH]3)nn1)OCCc1ccc(Cl)cc12]

184

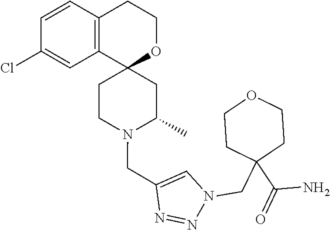 [SMILES: C[C@H]1C[C@@]2(CCN1Cc1cn(CC3(C(N)=O)CCOCC3)nn1)OCCc1ccc(Cl)cc12]

185

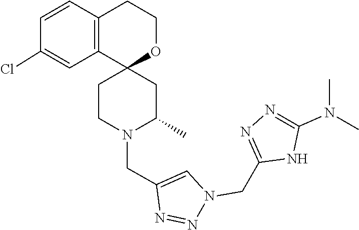 [SMILES: C[C@H]1C[C@@]2(CCN1Cc1cn(Cc3nnc(N(C)C)[nH]3)nn1)OCCc1ccc(Cl)cc12]

186

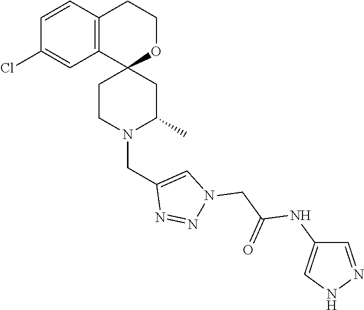 [SMILES: C[C@H]1C[C@@]2(CCN1Cc1cn(CC(=O)Nc3cn[nH]c3)nn1)OCCc1ccc(Cl)cc12]

187

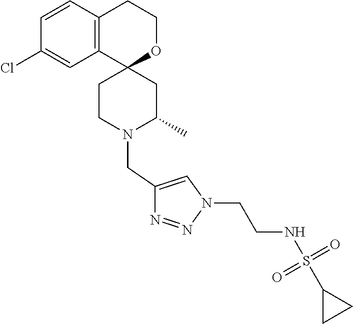 [SMILES: C[C@H]1C[C@@]2(CCN1Cc1cn(CCNS(=O)(=O)C3CC3)nn1)OCCc1ccc(Cl)cc12]

188

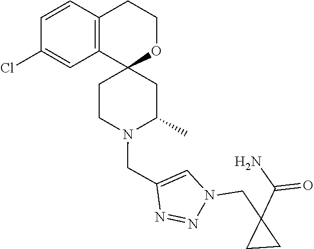 [SMILES: C[C@H]1C[C@@]2(CCN1Cc1cn(CC3(C(N)=O)CC3)nn1)OCCc1ccc(Cl)cc12]

189

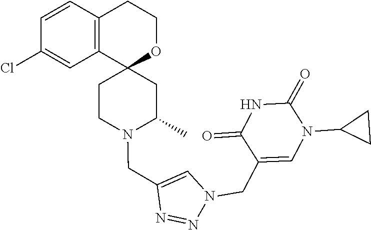 [SMILES: C[C@H]1C[C@@]2(CCN1Cc1cn(Cc3cn(C4CC4)c(=O)[nH]c3=O)nn1)OCCc1ccc(Cl)cc12]

190

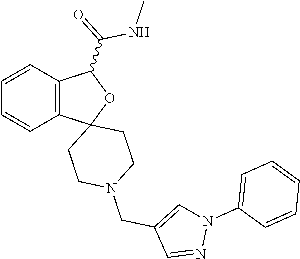 [SMILES: CNC(=O)C1OC2(CCN(Cc3cnn(-c4ccccc4)c3)CC2)c2ccccc21]

191

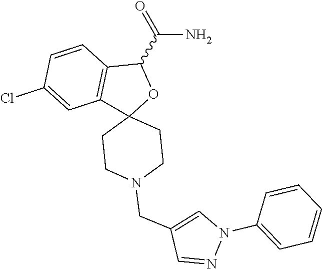 [SMILES: NC(=O)C1OC2(CCN(Cc3cnn(-c4ccccc4)c3)CC2)c2cc(Cl)ccc21]

192

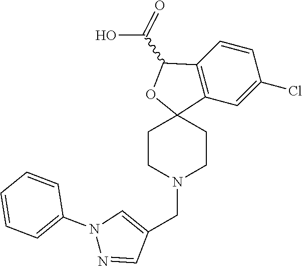 [SMILES: O=C(O)C1OC2(CCN(Cc3cnn(-c4ccccc4)c3)CC2)c2cc(Cl)ccc21]

193

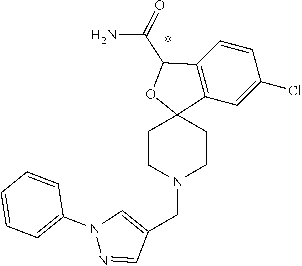 [SMILES: NC(=O)C1OC2(CCN(Cc3cnn(-c4ccccc4)c3)CC2)c2cc(Cl)ccc21]

194

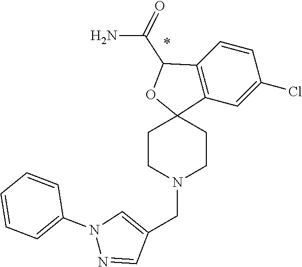 [SMILES: NC(=O)C1OC2(CCN(Cc3cnn(-c4ccccc4)c3)CC2)c2cc(Cl)ccc21]

195

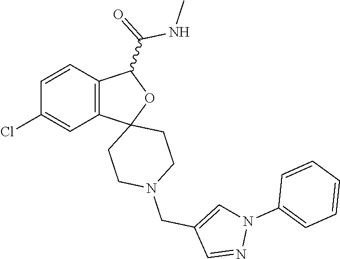 [SMILES: CNC(=O)C1OC2(CCN(Cc3cnn(-c4ccccc4)c3)CC2)c2cc(Cl)ccc21]

196

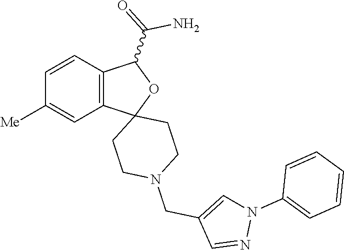 [SMILES: Cc1ccc2c(c1)C1(CCN(Cc3cnn(-c4ccccc4)c3)CC1)OC2C(N)=O]

197

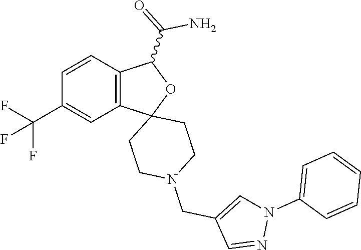 [SMILES: NC(=O)C1OC2(CCN(Cc3cnn(-c4ccccc4)c3)CC2)c2cc(C(F)(F)F)ccc21]

198

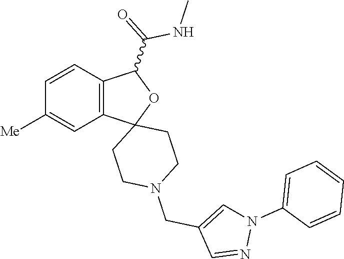 [SMILES: CNC(=O)C1OC2(CCN(Cc3cnn(-c4ccccc4)c3)CC2)c2cc(C)ccc21]

199

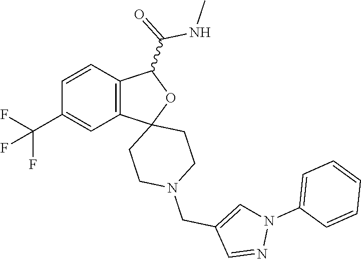 [SMILES: CNC(=O)C1OC2(CCN(Cc3cnn(-c4ccccc4)c3)CC2)c2cc(C(F)(F)F)ccc21]

200

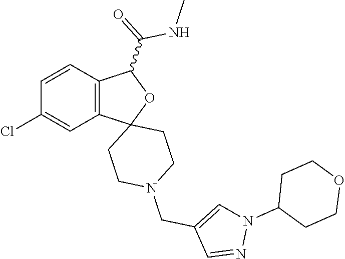 [SMILES: CNC(=O)C1OC2(CCN(Cc3cnn(C4CCOCC4)c3)CC2)c2cc(Cl)ccc21]

201

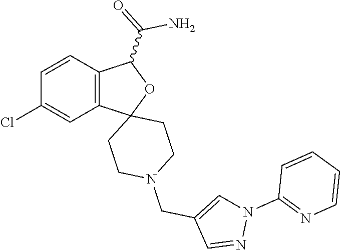 [SMILES: NC(=O)C1OC2(CCN(Cc3cnn(-c4ccccn4)c3)CC2)c2cc(Cl)ccc21]

202

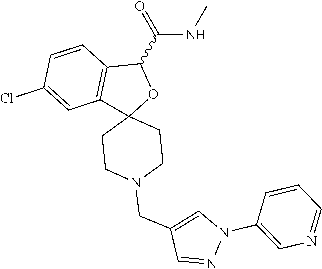 [SMILES: CNC(=O)C1OC2(CCN(Cc3cnn(-c4cccnc4)c3)CC2)c2cc(Cl)ccc21]

203

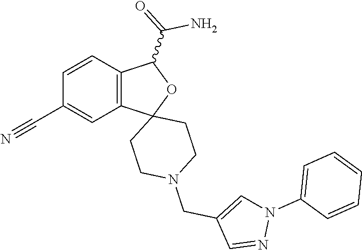 [SMILES: N#Cc1ccc2c(c1)C1(CCN(Cc3cnn(-c4ccccc4)c3)CC1)OC2C(N)=O]

204

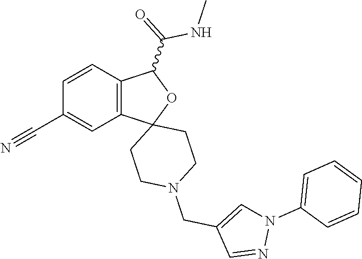 [SMILES: CNC(=O)C1OC2(CCN(Cc3cnn(-c4ccccc4)c3)CC2)c2cc(C#N)ccc21]

205

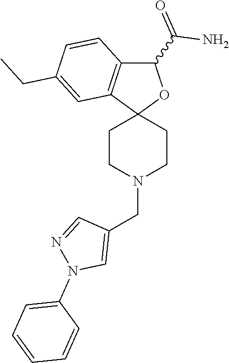 [SMILES: CCc1ccc2c(c1)C1(CCN(Cc3cnn(-c4ccccc4)c3)CC1)OC2C(N)=O]

206

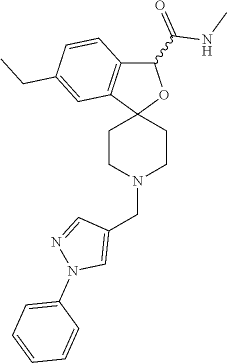 [SMILES: CCc1ccc2c(c1)C1(CCN(Cc3cnn(-c4ccccc4)c3)CC1)OC2C(=O)NC]

207

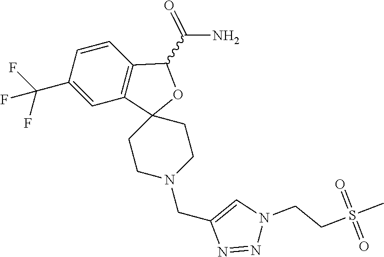 [SMILES: CS(=O)(=O)CCn1cc(CN2CCC3(CC2)OC(C(N)=O)c2ccc(C(F)(F)F)cc23)nn1]

208

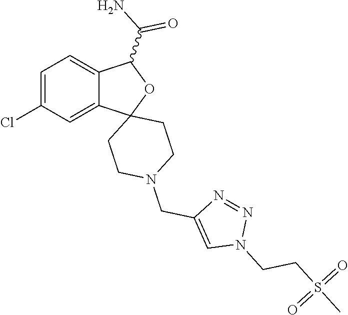 [SMILES: CS(=O)(=O)CCn1cc(CN2CCC3(CC2)OC(C(N)=O)c2ccc(Cl)cc23)nn1]

209

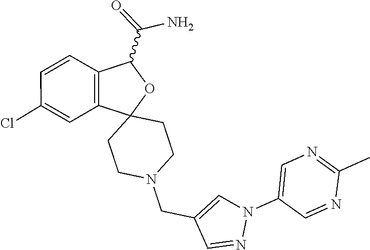 [SMILES: Cc1ncc(-n2cc(CN3CCC4(CC3)OC(C(N)=O)c3ccc(Cl)cc34)cn2)cn1]

210

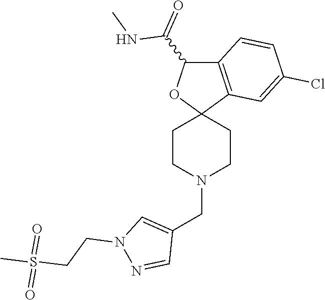 [SMILES: CNC(=O)C1OC2(CCN(Cc3cnn(CCS(C)(=O)=O)c3)CC2)c2cc(Cl)ccc21]

211

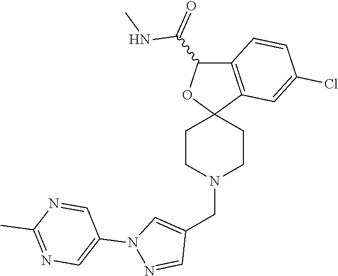 [SMILES: CNC(=O)C1OC2(CCN(Cc3cnn(-c4cnc(C)nc4)c3)CC2)c2cc(Cl)ccc21]

212

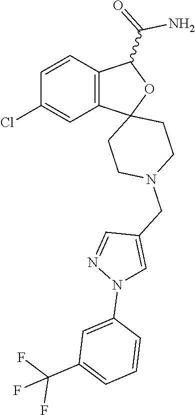 [SMILES: NC(=O)C1OC2(CCN(Cc3cnn(-c4cccc(C(F)(F)F)c4)c3)CC2)c2cc(Cl)ccc21]

213

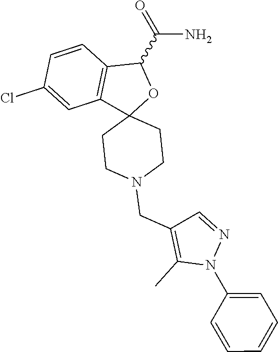 [SMILES: Cc1c(CN2CCC3(CC2)OC(C(N)=O)c2ccc(Cl)cc23)cnn1-c1ccccc1]

214

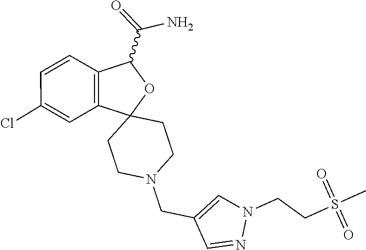 [SMILES: CS(=O)(=O)CCn1cc(CN2CCC3(CC2)OC(C(N)=O)c2ccc(Cl)cc23)cn1]

215

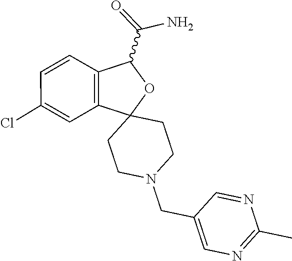 [SMILES: Cc1ncc(CN2CCC3(CC2)OC(C(N)=O)c2ccc(Cl)cc23)cn1]

216

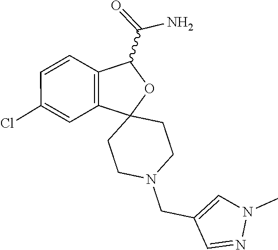 [SMILES: Cn1cc(CN2CCC3(CC2)OC(C(N)=O)c2ccc(Cl)cc23)cn1]

217

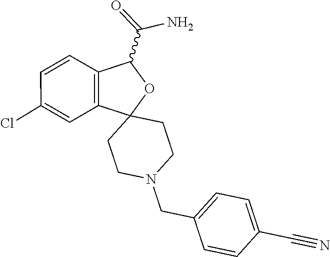 [SMILES: N#Cc1ccc(CN2CCC3(CC2)OC(C(N)=O)c2ccc(Cl)cc23)cc1]

218

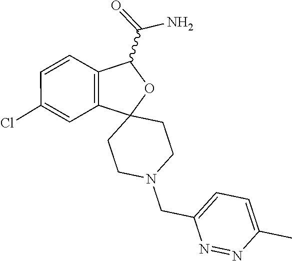 [SMILES: Cc1ccc(CN2CCC3(CC2)OC(C(N)=O)c2ccc(Cl)cc23)nn1]

219

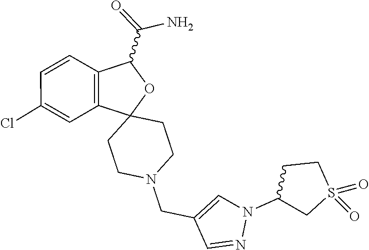 [SMILES: NC(=O)C1OC2(CCN(Cc3cnn(C4CCS(=O)(=O)C4)c3)CC2)c2cc(Cl)ccc21]

220

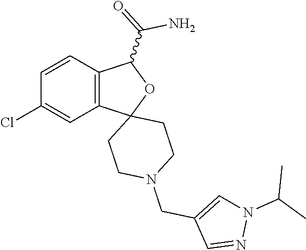 [SMILES: CC(C)n1cc(CN2CCC3(CC2)OC(C(N)=O)c2ccc(Cl)cc23)cn1]

221

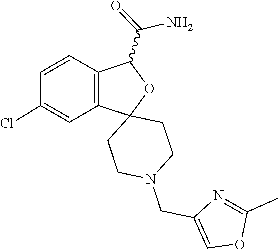 [SMILES: Cc1nc(CN2CCC3(CC2)OC(C(N)=O)c2ccc(Cl)cc23)co1]

222

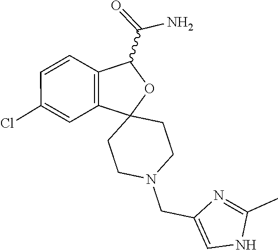 [SMILES: Cc1nc(CN2CCC3(CC2)OC(C(N)=O)c2ccc(Cl)cc23)c[nH]1]

223

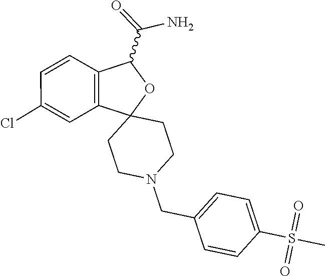 [SMILES: CS(=O)(=O)c1ccc(CN2CCC3(CC2)OC(C(N)=O)c2ccc(Cl)cc23)cc1]

224

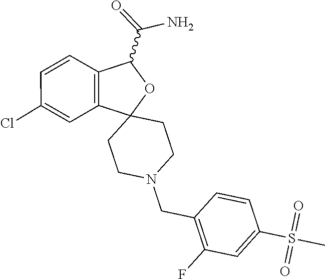 [SMILES: CS(=O)(=O)c1ccc(CN2CCC3(CC2)OC(C(N)=O)c2ccc(Cl)cc23)c(F)c1]

225

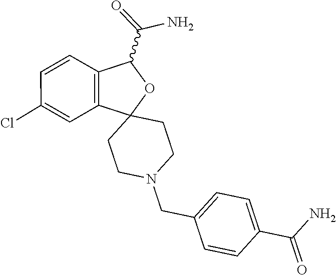 [SMILES: NC(=O)c1ccc(CN2CCC3(CC2)OC(C(N)=O)c2ccc(Cl)cc23)cc1]

226

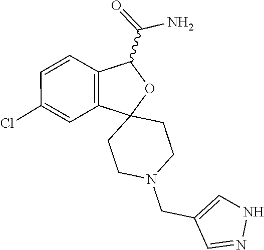 [SMILES: NC(=O)C1OC2(CCN(Cc3cn[nH]c3)CC2)c2cc(Cl)ccc21]

227

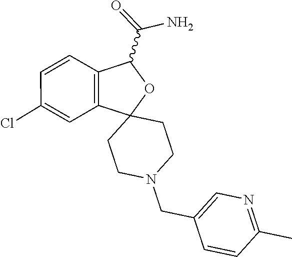 [SMILES: Cc1ccc(CN2CCC3(CC2)OC(C(N)=O)c2ccc(Cl)cc23)cn1]

228

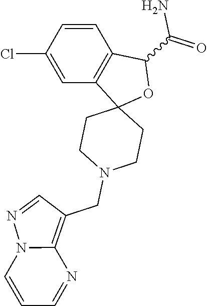 [SMILES: NC(=O)C1OC2(CCN(Cc3cnn4cccnc34)CC2)c2cc(Cl)ccc21]

229

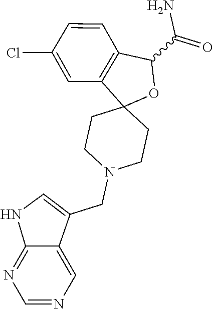 [SMILES: NC(=O)C1OC2(CCN(Cc3c[nH]c4ncncc34)CC2)c2cc(Cl)ccc21]

230

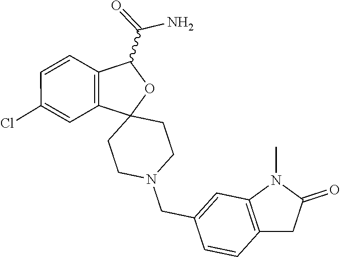 [SMILES: CN1C(=O)Cc2ccc(CN3CCC4(CC3)OC(C(N)=O)c3ccc(Cl)cc34)cc21]

231

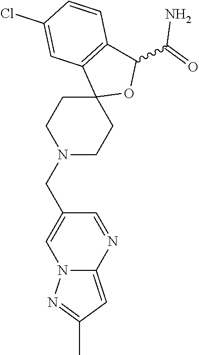 [SMILES: Cc1cc2ncc(CN3CCC4(CC3)OC(C(N)=O)c3ccc(Cl)cc34)cn2n1]

232

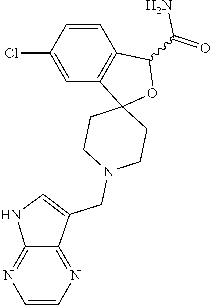 [SMILES: NC(=O)C1OC2(CCN(Cc3c[nH]c4nccnc34)CC2)c2cc(Cl)ccc21]

233

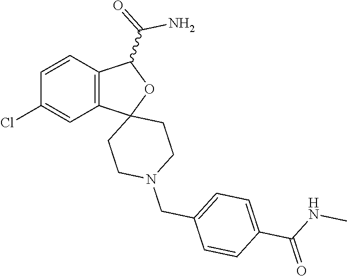 [SMILES: CNC(=O)c1ccc(CN2CCC3(CC2)OC(C(N)=O)c2ccc(Cl)cc23)cc1]

234

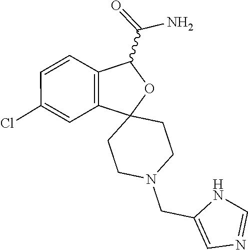 [SMILES: NC(=O)C1OC2(CCN(Cc3cnc[nH]3)CC2)c2cc(Cl)ccc21]

235

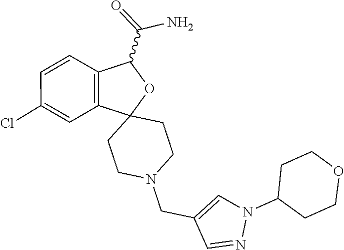 [SMILES: NC(=O)C1OC2(CCN(Cc3cnn(C4CCOCC4)c3)CC2)c2cc(Cl)ccc21]

236

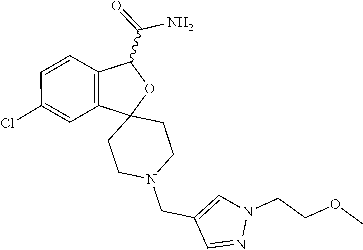 [SMILES: COCCn1cc(CN2CCC3(CC2)OC(C(N)=O)c2ccc(Cl)cc23)cn1]

237

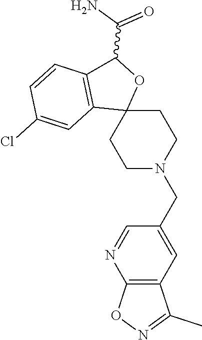 [SMILES: Cc1noc2ncc(CN3CCC4(CC3)OC(C(N)=O)c3ccc(Cl)cc34)cc12]

238

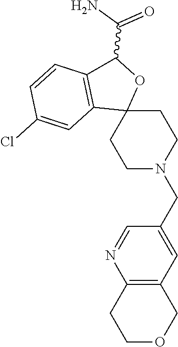 [SMILES: NC(=O)C1OC2(CCN(Cc3cnc4c(c3)COCC4)CC2)c2cc(Cl)ccc21]

239

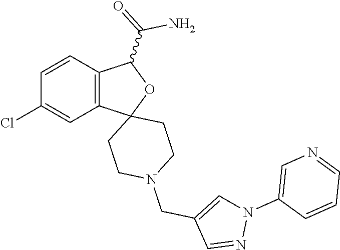 [SMILES: NC(=O)C1OC2(CCN(Cc3cnn(-c4cccnc4)c3)CC2)c2cc(Cl)ccc21]

240

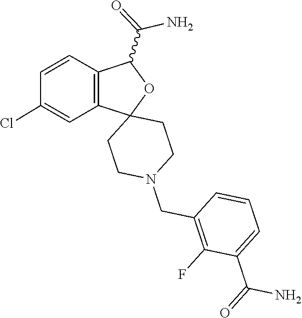 [SMILES: NC(=O)c1cccc(CN2CCC3(CC2)OC(C(N)=O)c2ccc(Cl)cc23)c1F]

241

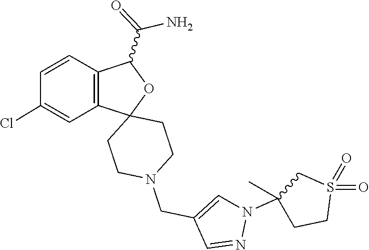 [SMILES: CC1(n2cc(CN3CCC4(CC3)OC(C(N)=O)c3ccc(Cl)cc34)cn2)CCS(=O)(=O)C1]

242

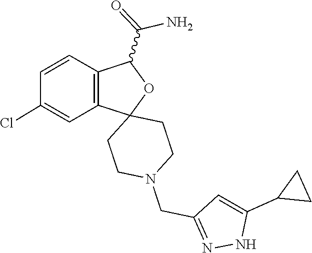 [SMILES: NC(=O)C1OC2(CCN(Cc3cc(C4CC4)[nH]n3)CC2)c2cc(Cl)ccc21]

243

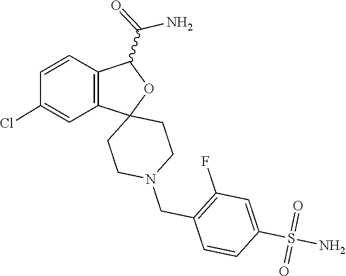 [SMILES: NC(=O)C1OC2(CCN(Cc3ccc(S(N)(=O)=O)cc3F)CC2)c2cc(Cl)ccc21]

244

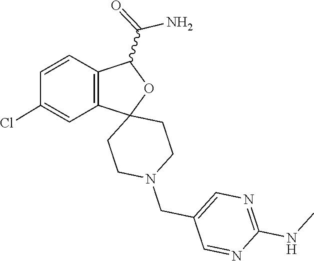 [SMILES: CNc1ncc(CN2CCC3(CC2)OC(C(N)=O)c2ccc(Cl)cc23)cn1]

245

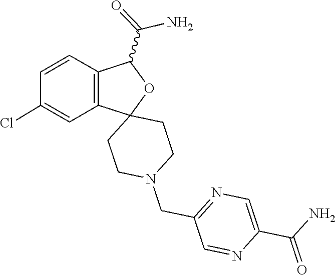 [SMILES: NC(=O)c1cnc(CN2CCC3(CC2)OC(C(N)=O)c2ccc(Cl)cc23)cn1]

246

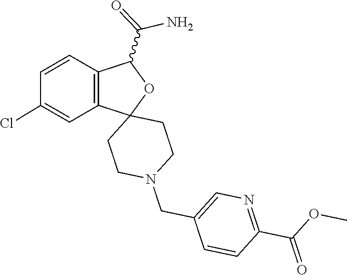 [SMILES: COC(=O)c1ccc(CN2CCC3(CC2)OC(C(N)=O)c2ccc(Cl)cc23)cn1]

247

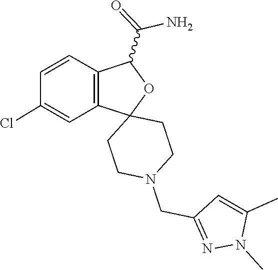 [SMILES: Cc1cc(CN2CCC3(CC2)OC(C(N)=O)c2ccc(Cl)cc23)nn1C]

248

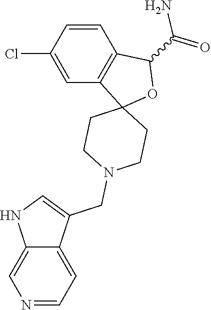 [SMILES: NC(=O)C1OC2(CCN(Cc3c[nH]c4cnccc34)CC2)c2cc(Cl)ccc21]

249

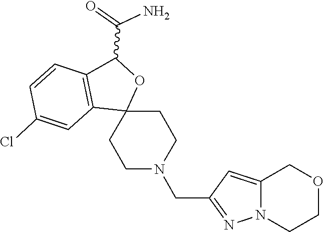 [SMILES: NC(=O)C1OC2(CCN(Cc3cc4n(n3)CCOC4)CC2)c2cc(Cl)ccc21]

250

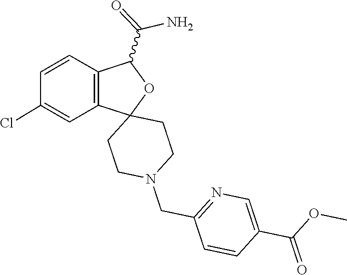 [SMILES: COC(=O)c1ccc(CN2CCC3(CC2)OC(C(N)=O)c2ccc(Cl)cc23)nc1]

251

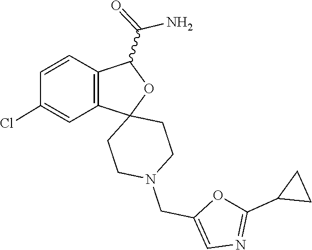 [SMILES: NC(=O)C1OC2(CCN(Cc3cnc(C4CC4)o3)CC2)c2cc(Cl)ccc21]

252

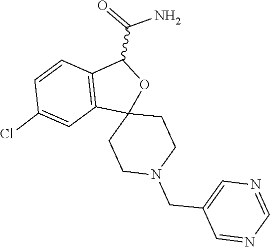 [SMILES: NC(=O)C1OC2(CCN(Cc3cncnc3)CC2)c2cc(Cl)ccc21]

253

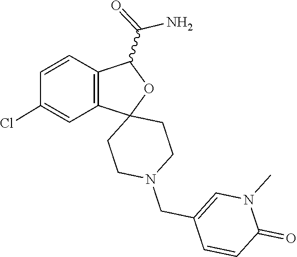 [SMILES: Cn1cc(CN2CCC3(CC2)OC(C(N)=O)c2ccc(Cl)cc23)ccc1=O]

254

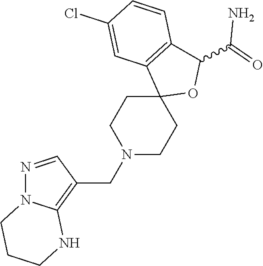 [SMILES: NC(=O)C1OC2(CCN(Cc3cnn4c3NCCC4)CC2)c2cc(Cl)ccc21]

255

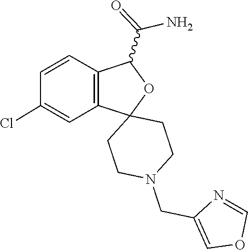 [SMILES: NC(=O)C1OC2(CCN(Cc3cocn3)CC2)c2cc(Cl)ccc21]

256

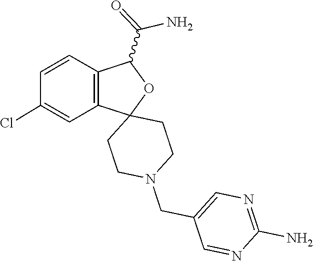 [SMILES: NC(=O)C1OC2(CCN(Cc3cnc(N)nc3)CC2)c2cc(Cl)ccc21]

257

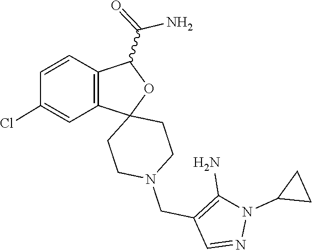 [SMILES: NC(=O)C1OC2(CCN(Cc3cnn(C4CC4)c3N)CC2)c2cc(Cl)ccc21]

258

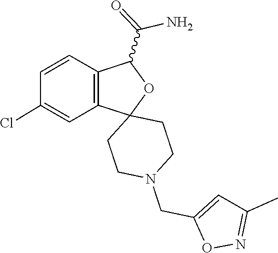 [SMILES: Cc1cc(CN2CCC3(CC2)OC(C(N)=O)c2ccc(Cl)cc23)on1]

259

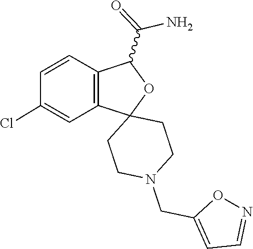 [SMILES: NC(=O)C1OC2(CCN(Cc3ccno3)CC2)c2cc(Cl)ccc21]

260

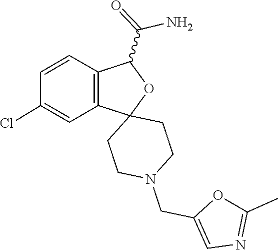 [SMILES: Cc1ncc(CN2CCC3(CC2)OC(C(N)=O)c2ccc(Cl)cc23)o1]

261

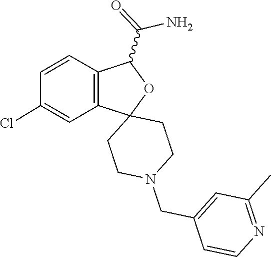 [SMILES: Cc1cc(CN2CCC3(CC2)OC(C(N)=O)c2ccc(Cl)cc23)ccn1]

262

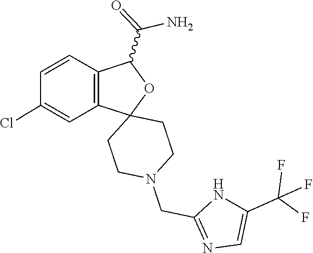 [SMILES: NC(=O)C1OC2(CCN(Cc3ncc(C(F)(F)F)[nH]3)CC2)c2cc(Cl)ccc21]

263

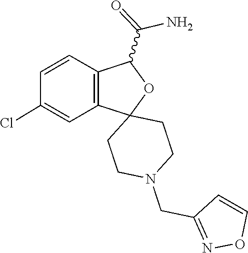 [SMILES: NC(=O)C1OC2(CCN(Cc3ccon3)CC2)c2cc(Cl)ccc21]

264

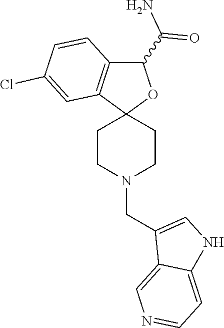 [SMILES: NC(=O)C1OC2(CCN(Cc3c[nH]c4ccncc34)CC2)c2cc(Cl)ccc21]

265

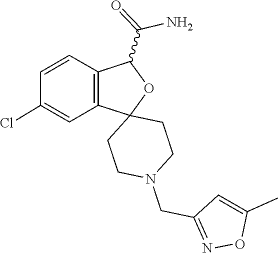 [SMILES: Cc1cc(CN2CCC3(CC2)OC(C(N)=O)c2ccc(Cl)cc23)no1]

266

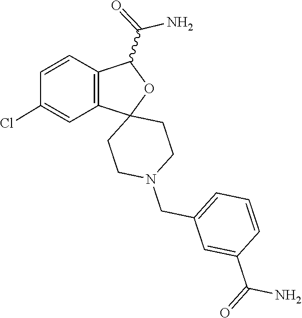 [SMILES: NC(=O)c1cccc(CN2CCC3(CC2)OC(C(N)=O)c2ccc(Cl)cc23)c1]

267

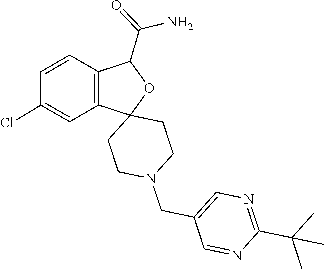 [SMILES: CC(C)(C)c1ncc(CN2CCC3(CC2)OC(C(N)=O)c2ccc(Cl)cc23)cn1]

268

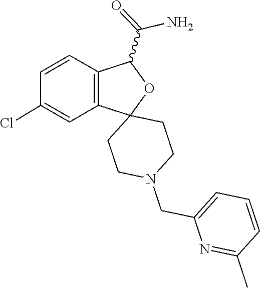 [SMILES: Cc1cccc(CN2CCC3(CC2)OC(C(N)=O)c2ccc(Cl)cc23)n1]

269

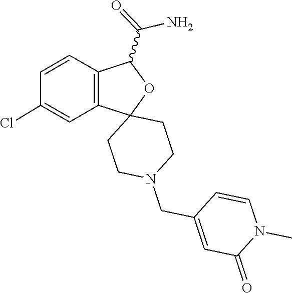 [SMILES: Cn1ccc(CN2CCC3(CC2)OC(C(N)=O)c2ccc(Cl)cc23)cc1=O]

270

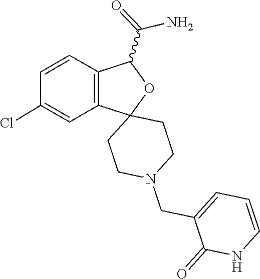 [SMILES: NC(=O)C1OC2(CCN(Cc3ccc[nH]c3=O)CC2)c2cc(Cl)ccc21]

271

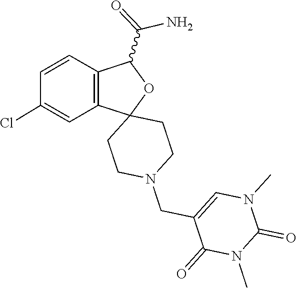 [SMILES: Cn1cc(CN2CCC3(CC2)OC(C(N)=O)c2ccc(Cl)cc23)c(=O)n(C)c1=O]

272

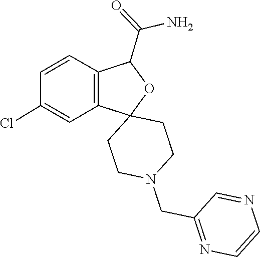 [SMILES: NC(=O)C1OC2(CCN(Cc3cnccn3)CC2)c2cc(Cl)ccc21]

273

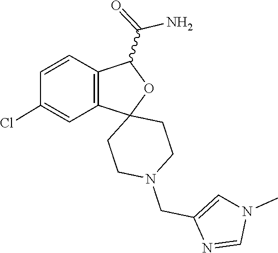 [SMILES: Cn1cnc(CN2CCC3(CC2)OC(C(N)=O)c2ccc(Cl)cc23)c1]

274

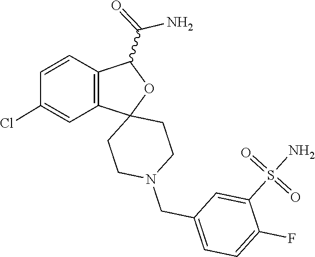 [SMILES: NC(=O)C1OC2(CCN(Cc3ccc(F)c(S(N)(=O)=O)c3)CC2)c2cc(Cl)ccc21]

275

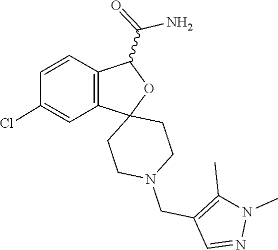 [SMILES: Cc1c(CN2CCC3(CC2)OC(C(N)=O)c2ccc(Cl)cc23)cnn1C]

276

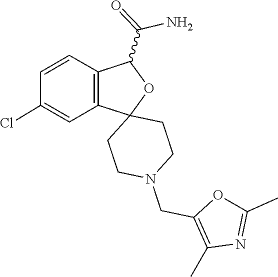 [SMILES: Cc1nc(C)c(CN2CCC3(CC2)OC(C(N)=O)c2ccc(Cl)cc23)o1]

277

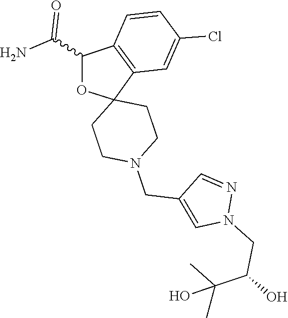 [SMILES: CC(C)(O)[C@@H](O)Cn1cc(CN2CCC3(CC2)OC(C(N)=O)c2ccc(Cl)cc23)cn1]

278

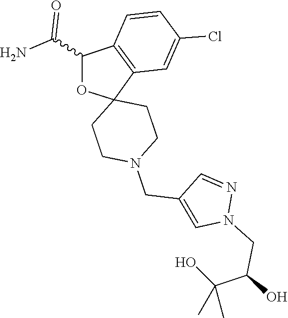 [SMILES: CC(C)(O)[C@H](O)Cn1cc(CN2CCC3(CC2)OC(C(N)=O)c2ccc(Cl)cc23)cn1]

279

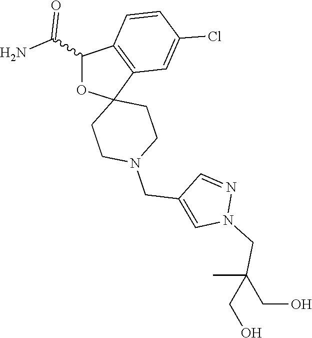 [SMILES: CC(CO)(CO)Cn1cc(CN2CCC3(CC2)OC(C(N)=O)c2ccc(Cl)cc23)cn1]

280

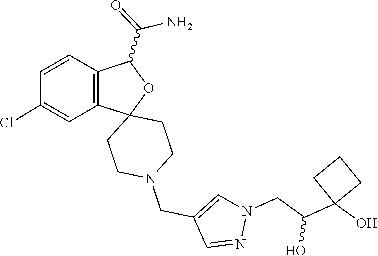 [SMILES: NC(=O)C1OC2(CCN(Cc3cnn(CC(O)C4(O)CCC4)c3)CC2)c2cc(Cl)ccc21]

281

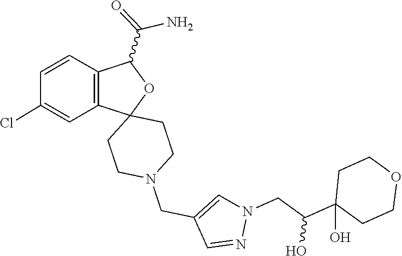 [SMILES: NC(=O)C1OC2(CCN(Cc3cnn(CC(O)C4(O)CCOCC4)c3)CC2)c2cc(Cl)ccc21]

282

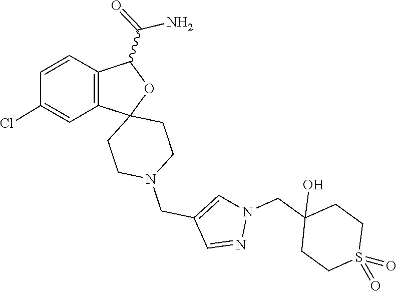 [SMILES: NC(=O)C1OC2(CCN(Cc3cnn(CC4(O)CCS(=O)(=O)CC4)c3)CC2)c2cc(Cl)ccc21]

283

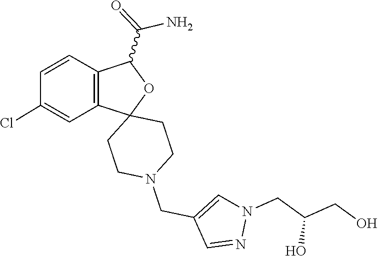 [SMILES: NC(=O)C1OC2(CCN(Cc3cnn(C[C@@H](O)CO)c3)CC2)c2cc(Cl)ccc21]

284

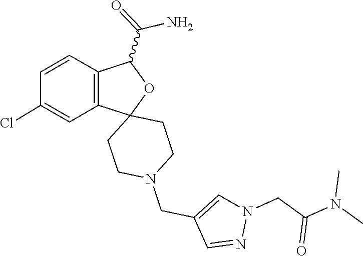 [SMILES: CN(C)C(=O)Cn1cc(CN2CCC3(CC2)OC(C(N)=O)c2ccc(Cl)cc23)cn1]

285

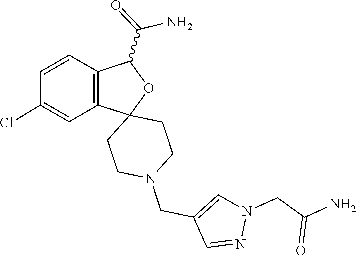 [SMILES: NC(=O)Cn1cc(CN2CCC3(CC2)OC(C(N)=O)c2ccc(Cl)cc23)cn1]

286

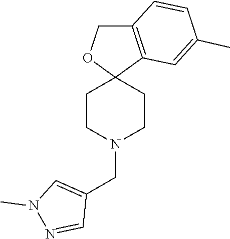

287

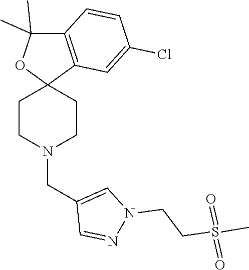

288

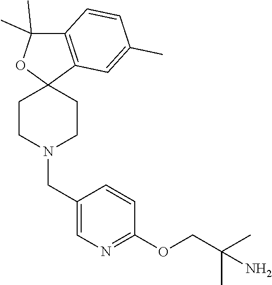 [SMILES: Cc1ccc2c(c1)C1(CCN(Cc3ccc(OCC(C)(C)N)nc3)CC1)OC2(C)C]

289

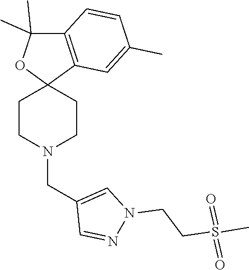

290

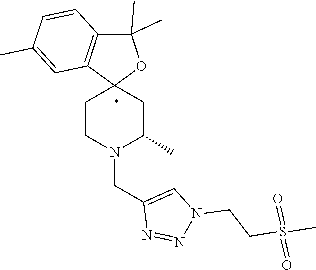

291

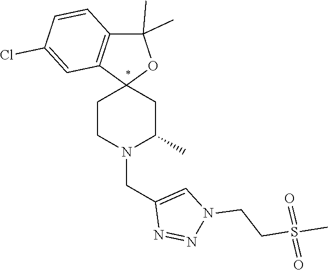 [SMILES: C[C@H]1CC2(CCN1Cc1cn(CCS(C)(=O)=O)nn1)OC(C)(C)c1ccc(Cl)cc12]

292

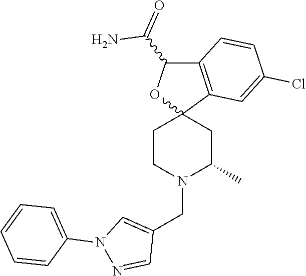 [SMILES: C[C@H]1CC2(CCN1Cc1cnn(-c3ccccc3)c1)OC(C(N)=O)c1ccc(Cl)cc12]

293

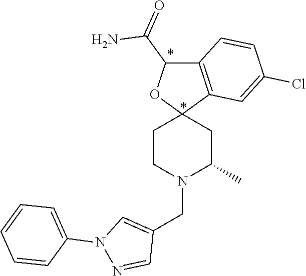 [SMILES: C[C@H]1CC2(CCN1Cc1cnn(-c3ccccc3)c1)OC(C(N)=O)c1ccc(Cl)cc12]

294

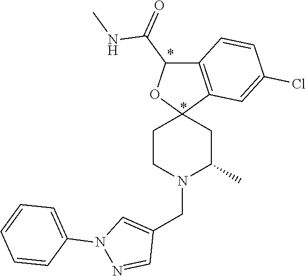 [SMILES: CNC(=O)C1OC2(CCN(Cc3cnn(-c4ccccc4)c3)[C@@H](C)C2)c2cc(Cl)ccc21]

295

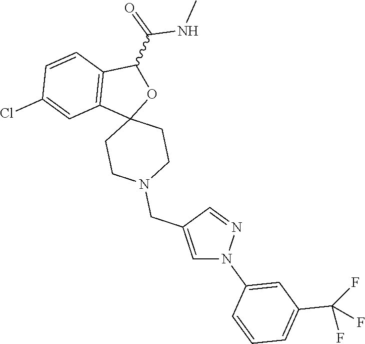 [SMILES: CNC(=O)C1OC2(CCN(Cc3cnn(-c4cccc(C(F)(F)F)c4)c3)CC2)c2cc(Cl)ccc21]

296

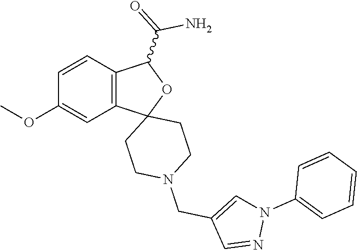 [SMILES: COc1ccc2c(c1)C1(CCN(Cc3cnn(-c4ccccc4)c3)CC1)OC2C(N)=O]

297

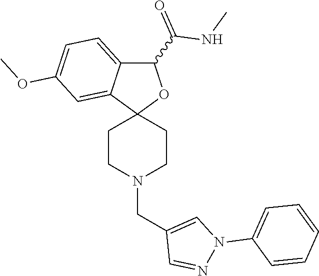 [SMILES: CNC(=O)C1OC2(CCN(Cc3cnn(-c4ccccc4)c3)CC2)c2cc(OC)ccc21]

298

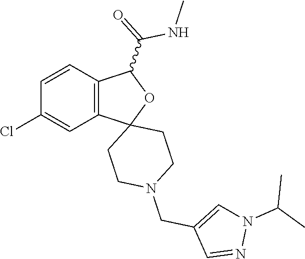 [SMILES: CNC(=O)C1OC2(CCN(Cc3cnn(C(C)C)c3)CC2)c2cc(Cl)ccc21]

299

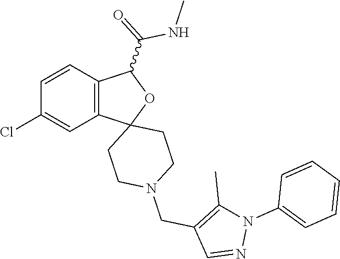 [SMILES: CNC(=O)C1OC2(CCN(Cc3cnn(-c4ccccc4)c3C)CC2)c2cc(Cl)ccc21]

300

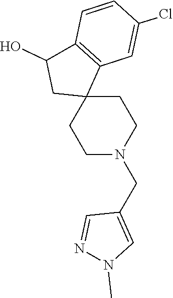 [SMILES: Cn1cc(CN2CCC3(CC2)CC(O)c2ccc(Cl)cc23)cn1]

301

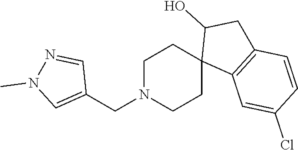 [SMILES: Cn1cc(CN2CCC3(CC2)c2cc(Cl)ccc2CC3O)cn1]

302

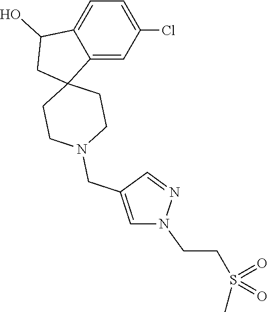 [SMILES: CS(=O)(=O)CCn1cc(CN2CCC3(CC2)CC(O)c2ccc(Cl)cc23)cn1]

303

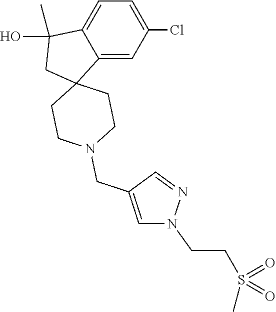 [SMILES: CC1(O)CC2(CCN(Cc3cnn(CCS(C)(=O)=O)c3)CC2)c2cc(Cl)ccc21]

304

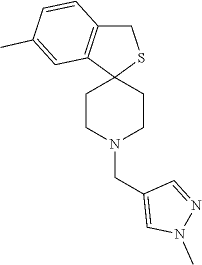 [SMILES: Cc1ccc2c(c1)C1(CCN(Cc3cnn(C)c3)CC1)SC2]

305

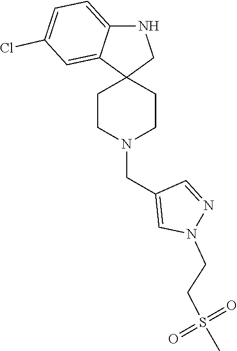 [SMILES: CS(=O)(=O)CCn1cc(CN2CCC3(CC2)CNc2ccc(Cl)cc23)cn1]

306

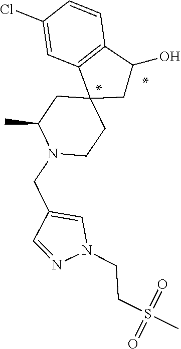

307

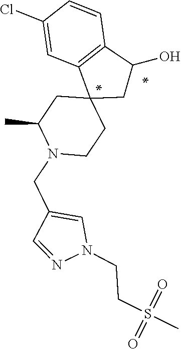

308

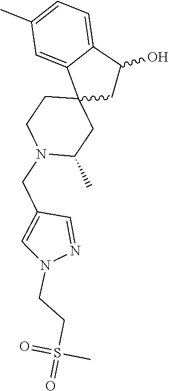 [SMILES: Cc1ccc2c(c1)C1(CCN(Cc3cnn(CCS(C)(=O)=O)c3)[C@@H](C)C1)CC2O]

309

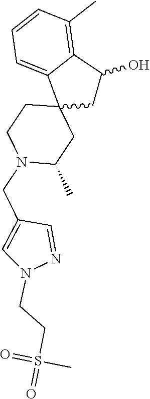 [SMILES: Cc1cccc2c1C(O)CC21CCN(Cc2cnn(CCS(C)(=O)=O)c2)[C@@H](C)C1]

310

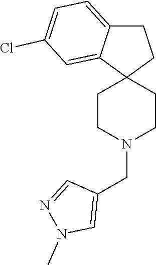 [SMILES: Cn1cc(CN2CCC3(CCc4ccc(Cl)cc43)CC2)cn1]

311

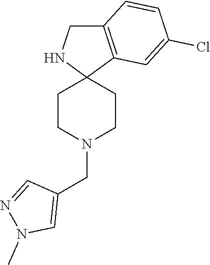 [SMILES: Cn1cc(CN2CCC3(CC2)NCc2ccc(Cl)cc23)cn1]

312

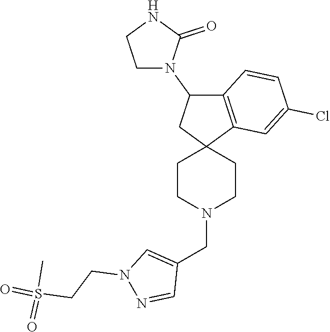 [SMILES: CS(=O)(=O)CCn1cc(CN2CCC3(CC2)CC(N2CCNC2=O)c2ccc(Cl)cc23)cn1]

313

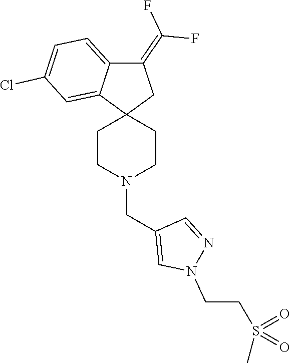 [SMILES: CS(=O)(=O)CCn1cc(CN2CCC3(CC2)CC(=C(F)F)c2ccc(Cl)cc23)cn1]

314

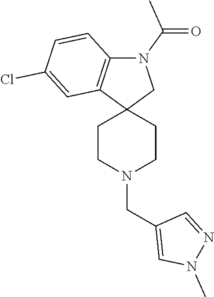 [SMILES: CC(=O)N1CC2(CCN(Cc3cnn(C)c3)CC2)c2cc(Cl)ccc21]

Some embodiments of the disclosure include derivatives of Compounds 1 to 299 or compounds of Formulae I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds or tautomers, or pharmaceutically acceptable salts of any of the foregoing. In some embodiments, the derivatives are silicon derivatives in which at least one carbon atom in a compound, tautomer, deuterated derivative, or pharmaceutically acceptable salt chosen from Compounds 1 to 299 or compounds of Formulae I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds or tautomers, and pharmaceutically acceptable salts of any of the foregoing, has been replaced by silicon. In some embodiments, the derivatives are boron derivatives, in which at least one carbon atom in a compound, tautomer, deuterated derivative, or pharmaceutically acceptable salt chosen from Compounds 1 to 299 or compounds of Formulae I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds or tautomers, and pharmaceutically acceptable salts of any of the foregoing, has been replaced by boron. In other embodiments, the derivatives are phosphorus derivatives, in which at least one carbon atom in a compound, tautomer, deuterated derivative, or pharmaceutically acceptable salt chosen from Compounds 1 to 299 or compounds of Formulae I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds or tautomers, and pharmaceutically acceptable salts of any of the foregoing, has been replaced by phosphorus.

In some embodiments, the derivative is a silicon derivative in which one carbon atom in a compound, tautomer, deuterated derivative, or pharmaceutically acceptable salt chosen from Compounds 1 to 299 or compounds of Formulae I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds or tautomers, and pharmaceutically acceptable salts of any of the foregoing, has been replaced by silicon or a silicon derivative (e.g., —Si(CH₃)₂— or —Si(OH)₂—). The carbon replaced by silicon may be a non-aromatic carbon. In other embodiments, a fluorine has been replaced by silicon derivative (e.g., —Si(CH₃)₃). In some embodiments, the silicon derivatives of the disclosure may include one or more hydrogen atoms replaced by deuterium. In some embodiments, a silicon derivative of compound, tautomer, deuterated derivative, or pharmaceutically acceptable salt chosen from Compounds 1 to 299 or compounds of Formulae I, IA, II, IIA, III, IV, IVA, IVB, IVC, V, VA, VB, VC, VI, VIA, VIB, VIC, VII, VIIA, VIII, VIIIA, IX, and IXA, a tautomer thereof, a deuterated derivative of that compound or tautomer, or a pharmaceutically acceptable salt of any of the foregoing, may have silicon incorporated into a heterocycle ring.

In some embodiments, the derivative is a boron derivative in which one carbon atom in a compound, tautomer, deuterated derivative, or pharmaceutically acceptable salt chosen from Compounds 1 to 299 or compounds of Formulae I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds or tautomers, and pharmaceutically acceptable salts of any of the foregoing, has been replaced by boron or a boron derivative.

In some embodiments, the derivative is a phosphorus derivative in which one carbon atom in a compound, tautomer, deuterated derivative, or pharmaceutically acceptable salt chosen from Compounds 1 to 299 or compounds of Formulae I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds or tautomers, and pharmaceutically acceptable salts of any of the foregoing, has been replaced by phosphorus or a phosphorus derivative.

Another aspect of the disclosure provides pharmaceutical compositions comprising at least one compound, tautomer, deuterated derivative, or pharmaceutically acceptable salt according to any one formula chosen from Formulae I, IA, II, IIA, III, IV, IVA, IVB, IVC, V, VA, VB, VC, VI, VIA, VIB, VIC, VII, VIIA, VIII, VIIIA, IX, and IXA, and Compounds 1 to 299, tautomers thereof, deuterated derivatives of those compounds or tautomers, and pharmaceutically acceptable salts of any of the foregoing. In some embodiments, the pharmaceutical composition comprising at least one compound, tautomer, deuterated derivative, or pharmaceutically acceptable salt chosen from Formulae I, IA, II, IIA, III, IV, IVA, IVB, IVC, V, VA, VB, VC, VI, VIA, VIB, VIC, VII, VIIA, VIII, VIIIA, IX, and IXA, Compounds 1 to 299, tautomers thereof, deuterated derivatives of those compounds or tautomers, and pharmaceutically acceptable salts of any of the foregoing is administered to a patient in need thereof.

A pharmaceutical composition may further comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, and lubricants.

It will also be appreciated that a pharmaceutical composition of this disclosure can be employed in combination therapies; that is, the pharmaceutical compositions described herein can further include at least one additional active therapeutic agent. Alternatively, a pharmaceutical composition comprising at least one compound, tautomer, deuterated derivative, or pharmaceutically acceptable salt chosen from compounds of Formulae I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds or tautomers, and pharmaceutically acceptable salts of any of the foregoing can be administered as a separate composition concurrently with, prior to, or subsequent to, a composition comprising at least one other active therapeutic agent. In some embodiments, a pharmaceutical composition comprising at least one compound, tautomer, deuterated derivative, or pharmaceutically acceptable salt chosen from Compounds 1 to 299, tautomers thereof, deuterated derivatives of those compounds or tautomers, and pharmaceutically acceptable salts of any of the foregoing can be administered as a separate composition concurrently with, prior to, or subsequent to, a composition comprising at least one other active therapeutic agent.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as, e.g., human serum albumin), buffer substances (such as, e.g., phosphates, glycine, sorbic acid, and potassium sorbate), partial glyceride mixtures of saturated vegetable fatty acids, water, salts, and electrolytes (such as, e.g., protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as, e.g., lactose, glucose, and sucrose), starches (such as, e.g., corn starch and potato starch), cellulose and its derivatives (such as, e.g., sodium carboxymethyl cellulose, ethyl cellulose, and cellulose acetate), powdered tragacanth, malt, gelatin, talc, excipients (such as, e.g., cocoa butter and suppository waxes), oils (such as, e.g., peanut oil, cottonseed oil, safflower oil, sesame oil, olive oil, corn oil, and soybean oil), glycols (such as, e.g., propylene glycol and polyethylene glycol), esters (such as, e.g., ethyl oleate and ethyl laurate), agar, buffering agents (such as, e.g., magnesium hydroxide and aluminum hydroxide), alginic acid, pyrogen-free water, isotonic saline, Ringer's solution, ethyl alcohol, phosphate buffer solutions, non-toxic compatible lubricants (such as, e.g., sodium lauryl sulfate and magnesium stearate), coloring agents, releasing agents, coating agents, sweetening agents, flavoring agents, perfuming agents, preservatives, and antioxidants.

### Use of Compounds and Compositions

In some embodiments of the disclosure, the compounds and the pharmaceutical compositions described herein are used to treat FSGS and/or NDKD. In some embodiments, FSGS is mediated by APOL1. In some embodiments, NDKD is mediated by APOL1.

In some embodiments of the disclosure, the compounds and the pharmaceutical compositions described herein are used to treat cancer. In some embodiments, the cancer is mediated by APOL1.

In some embodiments of the disclosure, the compounds and the pharmaceutical compositions described herein are used to treat pancreatic cancer. In some embodiments, the pancreatic cancer is mediated by APOL1.

In some embodiments, the methods of the disclosure comprise administering to a patient in need thereof at least one compound, tautomer, deuterated derivative, or pharmaceutically acceptable salt chosen from compounds of Formulae I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds and tautomers, and pharmaceutically acceptable salts of any of the foregoing. In some embodiments, the compound, tautomer, deuterated derivative, or pharmaceutically acceptable salt is chosen from Compounds 1 to 299, tautomer thereof, deuterated derivatives of those compounds or tautomers, and pharmaceutically acceptable salts of any of the foregoing. In some embodiments, said patient in need thereof possesses APOL1 genetic variants, i.e., GI: S342G:1384M and G2: N388del:Y389del.

Another aspect of the disclosure provides methods of inhibiting APOL1 activity comprising contacting said APOL1 with at least one compound, tautomer, deuterated derivative, or pharmaceutically acceptable salt chosen from compounds of Formulae I, IA, II, IIA, III, IV, IVA, IVB, IVC, V, VA, VB, VC, VI, VIA, VIB, VIC, VII, VIIA, VIII, VIIIA, IX, and IXA, tautomers thereof, deuterated derivatives of those compounds or tautomers, and pharmaceutically acceptable salts of any of the foregoing. In some embodiments, the methods of inhibiting APOL1 activity comprise contacting said APOL1 with at least one compound, tautomer, deuterated derivative, or pharmaceutically acceptable salt chosen from Compounds 1 to 299, tautomers thereof, deuterated derivatives of those compounds or tautomers, and pharmaceutically acceptable salts of any of the foregoing.

### EXAMPLES

In order that the disclosure described herein may be more fully understood, the following examples are set forth. It should be understood that these examples are for illustrative purposes only and are not to be construed as limiting this disclosure in any manner.

The compounds of the disclosure may be made according to standard chemical practices or as described herein. Throughout the following synthetic schemes and in the descriptions for preparing compounds of Formulae I, IA, II, IIA, III, IV, IVA, IVB, IVC, V, VA, VB, VC, VI, VIA, VIB, VIC, VII, VIIA, VIII, VIIIA, IX, and IXA, Compounds 1 to 299, tautomers thereof, deuterated derivatives of those compounds and tautomers, and pharmaceutically acceptable salts of any of the foregoing, the following abbreviations are used:

### Abbreviations

AcOH=acetic acid
ARP=assay ready plate
BF₃·OEt₂=boro trifluoride diethyl etherate
Boc₂O=di-tert-butyl dicarbonate
DAST=diethylaminosulfur trifluoride
DBU=1,8-diazabicyclo[5.4.0]undec-7-ene
DCE=1,2-dichloroethane
DCM=dichloromethane
DEE=diethyl ether
DIPEA=N,N-Diisopropylethylamine or N-ethyl-N-isopropyl-propan-2-amine
DMEM=Dulbecco's modified Eagle's medium
DMF=dimethylformamide
DMPU=N,N′-dimethylpropyleneurea
DMSO=dimethyl sulfoxide
dppb=1-4-bis[P(Ph)₂]-butane
ESI-MS=electrospray ionization mass spectrometry
EtOAc=ethyl acetate
EtOH=ethanol
FBS═fetal bovine serum
HATU=[dimethylamino(triazolo[4,5-b]pyridin-3-yloxy)methylene]-dimethyl-ammonium (Phosphorus Hexafluoride Ion)
IPA=isopropyl alcohol
IPAmine=isopropylamine
LCMS=liquid chromatography mass spectrometry
LED=light emitting diode
mCPBA=meta-chloroperoxybenzoic acid
MeCN=acetonitrile
Mel=methyl iodide
MeMgBr=methylmagnesium bromide
MeOH=methanol
MTBE or TBME=Methyl tert-butyl ether
n-BuLi=n-butyllithium
NMP=N-methyl pyrrolidine
NMR=nuclear magnetic resonance
Pd(dppf)Cl₂=[1,1′-Bis(diphenylphosphino)ferrocene]dichloropalladium(II)
Pd(PPh₃)₄=palladium-tetrakis(triphenylphosphine)
PP=polypropylene
PPh₃=triphenylphosphine
PTFE=polytetrafluoroethylene
PTSA=p-Toluenesulfonic acid monohydrate
rf=retention factor
rt=room temperature
SFC=supercritical fluid chromatography
STAB=sodium triacetoxyborohydride
T3P=2,4,6-Tripropyl-1,3,5,2,4,6-trioxatriphosphorinane-2,4,6-trioxide
TBAF=tetra-n-butylammonium fluoride
TBS=silyl ether
TBSCl=tert-butyldimethylsilyl chloride
TBTA=Tris((1-benzyl-4-triazolyl)methyl)amine
tBuBrettPhos Pd G3=[(2-Di-tert-butylphosphino-3,6-dimethoxy-2′,4′,6′-triisopropyl-1,1′-biphenyl)-2-(2′-amino-1,1′-biphenyl)]palladium(II) methanesulfonate
TEA=triethylamine
Tet=tetracycline
TFA or TFAA=trifluoroacetic acid
Tf₂O=triflic anhydride
THF=tetrahydrofuran
TLC=thin-layer chromatography
TMSCN=trimethylsilyl cyanide
VT=variable temperature

### Example 1. Synthesis of Compounds

All the specific and generic compounds, and the intermediates disclosed for making those compounds, are considered to be part of the disclosure disclosed herein.

### Compound 1

### 5-chloro-1′-[[1-(2-methylsulfonylethyl)pyrazol-4-yl]methyl]spiro[indoline-3,4′-piperidine]-2-one (1)

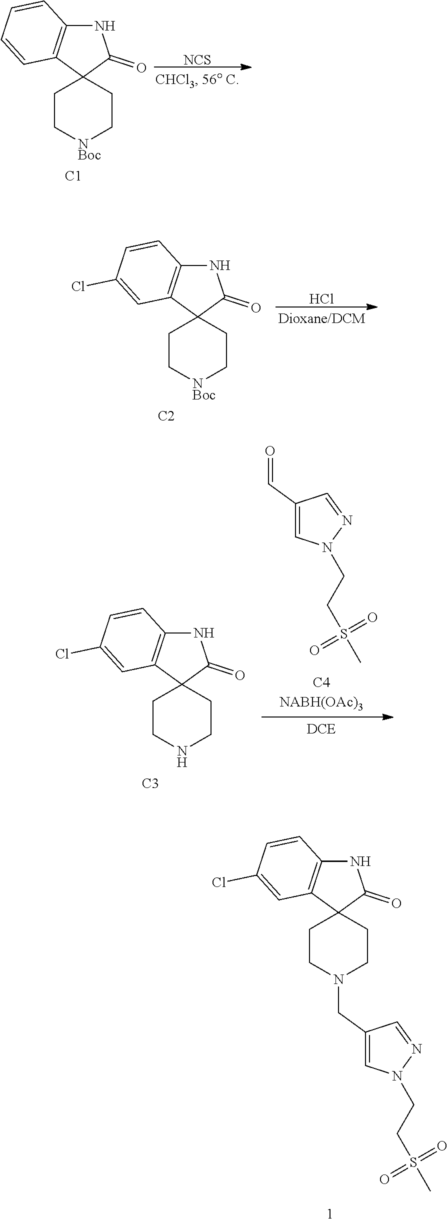

### Step 1: Preparation of tert-butyl 5-chloro-2-oxo-spiro[indoline-3,4′-piperidine]-1′-carboxylate (C2)

N-chlorosuccinimide (17.8 g, 133.3 mmol) was added to a solution of C1 (20.1 g, 66.58 mmol) in chloroform (300 mL) at room temperature. The resulting mixture was stirred at 56° C. for 24 h. N-chlorosuccinimide (4.3 g, 32.2 mmol) was added and the reaction mixture was continued to stir at 56° C. for 12 h. N-chlorosuccinimide (9 g, 67.4 mmol) was further added and the reaction mixture was continued to stir at 56° C. for 8 h. N-chlorosuccinimide (17 g, 127.3 mmol) was added and this time the reaction mixture was refluxed for 4 h. Reaction mixture was then brought to room temperature, washed with aqueous saturated solution of sodium bisulfite (200 mL) and brine (200 mL). Organic layer was concentrated in vacuo and the product was purified on silica gel chromatography (0-50% EtOAc:hexanes) which provided a mixture of starting material and product. The mixed material was purified by reversed-phase HPLC (Waters XSelect CSH C18 OBD Prep Column; 30×150 mm, 5 micron. Gradient: Acetonitrile in Water with 5 mM Hydrochloric Acid.). The product-containing fractions were pooled and concentrated to yield the title compound (C2) (2.18 g, 9%) as an off-white solid. ¹H NMR (500 MHz, DMSO-d6) δ 10.55 (s, 1H), 7.56 (d, J=2.2 Hz, 1H), 7.24 (dd, J=8.3, 2.2 Hz, 1H), 6.86 (d, J=8.2 Hz, 1H), 3.70-3.59 (m, 4H), 1.75-1.63 (m, 4H), 1.44 (s, 9H). LCMS m/z 337.5 [M+H]⁺.

### Step 2: Preparation of 5-chlorospiro[indoline-3,4′-piperidine]-2-one (C3)

A solution of DCM (5 mL) and HCl in dioxane (15 mL of 4 M, 60.0 mmol) was added to tert-butyl 5-chloro-2-oxo-spiro[indoline-3,4′-piperidine]-1′-carboxylate (C2) (2.25 g, 5.4 mmol) and the resulting mixture was stirred for 60 min at room temperature. Reaction mixture was then concentrated in vacuo and dried under high vacuum over night to provide the title compound (C3) dihydrochloride salt (2.1 g, 110%) which was used in the next steps without any further purification.

### Step 3: Preparation of 5-chloro-1′-[[1-(2-methylsulfonylethyl)pyrazol-4-yl]methyl]spiro[indoline-3,4′-piperidine]-2-one (1)

To a suspension of 5-chlorospiro[indoline-3,4′-piperidine]-2-one hydrochloride (C3) (199 mg, 0.656 mmol) and 1-(2-methylsulfonylethyl)pyrazole-4-carbaldehyde (C4) (157 mg, 0.668 mmol) in 3 ml DCE was added sodium triacetoxyborohydride (420 mg, 1.99 mmol) and the mixture was stirred at rt for 20 h.

At this time, the mixture was evaporated to remove volatiles. The material was purified by reversed-phase HPLC (Waters XSelect CSH C18 OBD Prep Column; 30×150 mm, 5 micron. Gradient: Acetonitrile in Water with 0.1% Trifluoroacetic Acid). The product-containing fractions were poole and the final product was converted to HCl salt by treating with HCl (4 M in dioxane) and the formed suspension was filtered and dried to yield the title compound 1 dihydrochloride salt (304 mg, 92%) ¹H NMR (300 MHz, Chloroform-d) δ 8.01 (d, J=0.7 Hz, 1H), 7.74 (s, 1H), 7.40-7.16 (m, 2H), 7.01-6.81 (m, 1H), 4.76-4.64 (m, 2H), 4.37 (s, 2H), 3.85-3.66 (m, 4H), 3.56-3.44 (m, 2H), 2.86 (s, 3H), 2.48-2.19 (m, 2H), 2.11-1.95 (m, 2H). LCMS m/z 423.05 [M+H]⁺ LCMS m/z 237.2 [M+H]⁺

Table 2. Compounds 2-36 were prepared in analogous fashion utilizing the listed intermediate as well as modified intermediates that one skilled in the art of organic chemistry could access, utilizing chemical transformations such as copper mediated cross coupling, Suzuki coupling, Negishi coupling, S_{N}Ar, S_{N}2, lithiation, acylation, halogenation, silylation, desilylation, 1,4-addition, and reduction.

TABLE 2
Compounds 2-36

| Compound | Aldehyde | Structure | ¹H NMR, LCMS M + H |
|---|---|---|---|
| 2 | 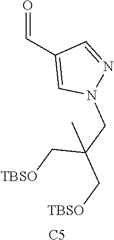 | 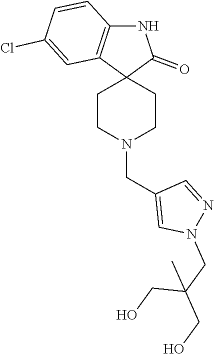 [SMILES: CC(CO)(CO)Cn1cc(CN2CCC3(CC2)C(=O)Nc2ccc(Cl)cc23)cn1] | ¹H NMR (400 MHz, Chloroform-d) δ 7.69 (d, J = 0.7 Hz, 1H), 7.52 (d, J = 0.7 Hz, 1H), 7.32 (d, J = 2.1 Hz, 1H), 7.21 (dd, J= 8.3, 2.1 Hz, 1H), 6.87 (d, J = 8.3 Hz, 1H), 4.14 (s, 2H), 3.73 (s, 2H), 3.44- 3.33 (m, 4H), 3.04 (d, J = 8.9 Hz, 2H), 2.86-2.76 (m, 2H), 2.02-1.92 (m, 2H), 1.92-1.83 (m, 2H), 0.84 (s, 3H). LCMS m/z 419.28 [M + H]⁺ |
| 3 | 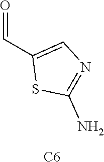 | 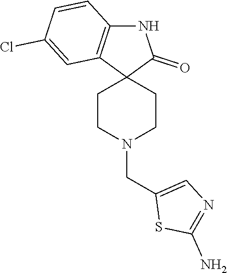 [SMILES: Nc1ncc(CN2CCC3(CC2)C(=O)Nc2ccc(Cl)cc23)s1] | LCMS m/z 349.2 [M + H]⁺ |
| 4 | 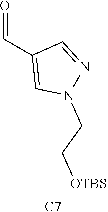 [SMILES: CC(C)(C)[Si](C)(C)OCCn1cc(C=O)cn1 ; "C7"] | 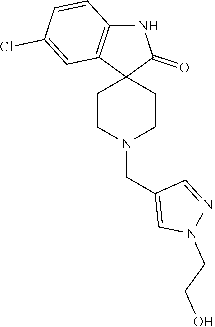 [SMILES: O=C1Nc2ccc(Cl)cc2C12CCN(Cc1cnn(CCO)c1)CC2] | ¹H NMR (400 MHz, Chloroform-d) δ 8.04 (d, J = 9.2 Hz, 1H), 7.82 (dd, J = 12.7, 0.7 Hz, 1H), 7.76-7.21 (m, 2H), 7.01- 6.82 (m, 1H), 4.46 (d, J = 65.5 Hz, 2H), 4.30 (t, J = 5.2 Hz, 2H), 3.91 (td, J = 5.4, 2.8 Hz, 2H), 3.80 (td, J = 13.2, 3.1 Hz, 2H), 3.55-3.36 (m, 2H), 2.40- 2.18 (m, 2H), 2.03 (s, 4H). LCMS m/z 360.98 [M + H]⁺ |
| 5 | 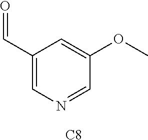 | 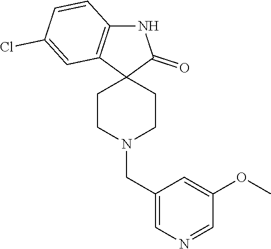 [SMILES: COc1cncc(CN2CCC3(CC2)C(=O)Nc2ccc(Cl)cc23)c1] | LCMS m/z 358.2 [M + H]⁺ |
| 6 | 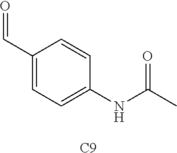 | 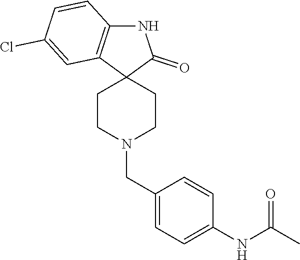 [SMILES: CC(=O)Nc1ccc(CN2CCC3(CC2)C(=O)Nc2ccc(Cl)cc23)cc1] | LCMS m/z 384.0 [M + H]⁺ |
| 7 | 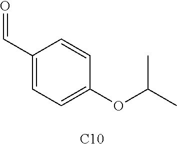 | 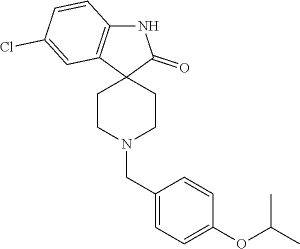 [SMILES: CC(C)Oc1ccc(CN2CCC3(CC2)C(=O)Nc2ccc(Cl)cc23)cc1] | (500 MHz, DMSO-d6) δ 10.76 (d, J = 47.6 Hz, 1H), 9.69 (s, 1H), 7.49 (t, J = 8.6 Hz, 2H), 7.37- 7.26 (m, 1H), 7.06-6.99 (m, 2H), 6.91 (dd, J = 25.8, 8.3 Hz, 1H), 4.71- 4.64 (m, 1H), 4.51 (d, J = 5.3 Hz, 1H), 4.37 (d, J = 4.8 Hz, 1H), 3.56 (dt, J = 21.4, 11.5 Hz, 2H), 3.35-3.26 (m, 2H), 2.24 (t, J = 15.9 Hz, 1H), 2.10 (t, J = 14.6 Hz, 1H), 1.97 (d, J = 14.6 Hz, |
| | | | 1H), 1.89 (d, J = 14.6 Hz, |
| | | | 1H), |
| | | | 1.29 (s, 3H), 1.28 (s, 3H). |
| | | | LCMS m/z 384.9 [M + H]⁺ |
| 8 | 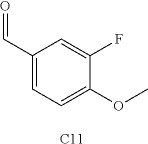 | 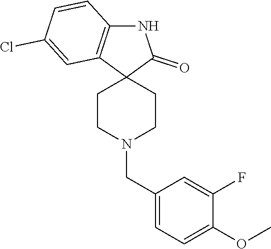 [SMILES: COc1ccc(CN2CCC3(CC2)C(=O)Nc2ccc(Cl)cc23)cc1F] | LCMS m/z 375.3 [M + H]⁺ |
| 9 | 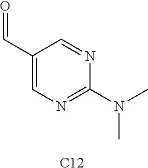 | 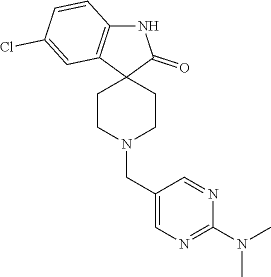 [SMILES: CN(C)c1ncc(CN2CCC3(CC2)C(=O)Nc2ccc(Cl)cc23)cn1] | LCMS m/z 372.3 [M + H]⁺ |
| 10 | 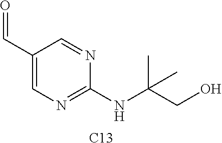 | 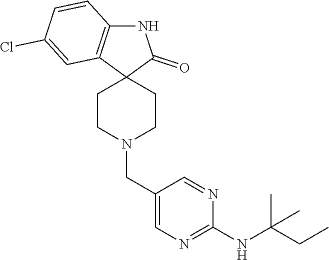 [SMILES: CCC(C)(C)Nc1ncc(CN2CCC3(CC2)C(=O)Nc2ccc(Cl)cc23)cn1] | ¹H NMR (300 MHz, Chloroform-d) δ 8.36 (s, 1H), 8.31 (s, 2H), 8.02 (s, 1H), 7.31 (d, J = 2.1 Hz, 1H), 7.22 (dd, J = 8.3, 2.1 Hz, 1H), 6.83 (d, J = 8.3 Hz, 1H), 5.66 (s, 1H), 3.70 (s, 1H), 3.67 (s, 2H), 3.13 (td, J = 11.0, 10.5, 3.4 Hz, 2H), 2.92 (dd, J = 11.5, 4.9 Hz, 2H), 2.19- 2.01 (m, 2H), 1.95 (d, J = 14.2 Hz, 2H), 1.40 (s, 6H). LCMS m/z 416.23 [M + H]⁺ |
| D11 | 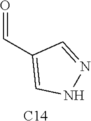 | 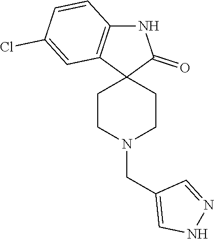 [SMILES: O=C1Nc2ccc(Cl)cc2C12CCN(Cc1cn[nH]c1)CC2] | LCMS m/z 317.1 [M + H]⁺ |
| 12 | 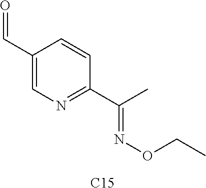 | 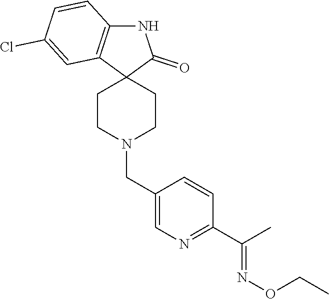 [SMILES: CCO/N=C(\C)c1ccc(CN2CCC3(CC2)C(=O)Nc2ccc(Cl)cc23)cn1] | ¹H NMR (400 MHz, DMSO-d6) δ 10.48 (s, 1H), 8.56 (s, 1H), 7.85 (d, J = 8.4 Hz, 1H), 7.79 (d, J = 8.4 Hz, 1H), 7.49 (s, 1H), 7.23 (d, J = 8.4 Hz, 1H), 6.84 (d, J = 8.4 Hz, 1H), 4.21 (q, J = 7.2 Hz, 2H), 3.67 (s, 2H), 2.85- 2.79 (m, 2H), 2.60-2.55 (m, 2H), 2.24 (s, 3H), 1.80-1.68 (m, 4H), 1.28 (t, J = 6.8 Hz, 3H). LCMS m/z 413.17 [M + H]⁺ |
| 13 | 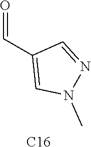 | 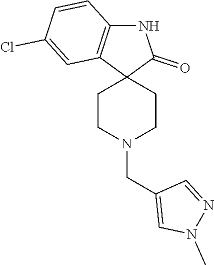 [SMILES: Cn1cc(CN2CCC3(CC2)C(=O)Nc2ccc(Cl)cc23)cn1] | ¹H NMR (400 MHz, DMSO-d6), 10.47 (bs, 1H), 7.598 (s, 1H), 7.408 (d, J =2, 1H), 7.331 (s, 1H), 7.225 (dd, J1=8.4, J2 = 2.4 1H), 6.835 (d, J = 8.4, 1H), 3.803 (s, 3H), 3.454 (s, 2H), 2.797- 2.741 (m, 2H), 2.550- 2.506 (m, 2H), 1.785- 1.733 (m, 2H), 1.680- 1.638 (m, 2H). LCMS m/z 331.00 [M + H]⁺ |
| 14 | 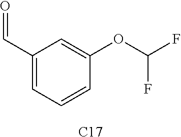 [SMILES: O=Cc1cccc(OC(F)F)c1 ; "C17"] | 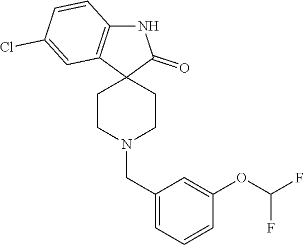 [SMILES: O=C1Nc2ccc(Cl)cc2C12CCN(Cc1cccc(OC(F)F)c1)CC2] | LCMS m/z 393.1 [M + H]⁺ |
| 15 | 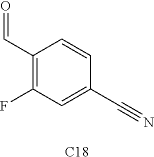 [SMILES: N#Cc1ccc(C=O)c(F)c1 ; "C18"] | 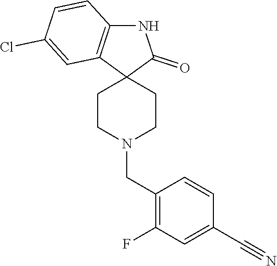 [SMILES: N#Cc1ccc(CN2CCC3(CC2)C(=O)Nc2ccc(Cl)cc23)c(F)c1] | LCMS m/z 369.9 [M + H]⁺ |
| 16 | 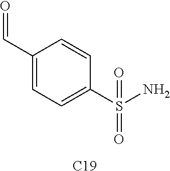 [SMILES: NS(=O)(=O)c1ccc(C=O)cc1 ; "C19"] | 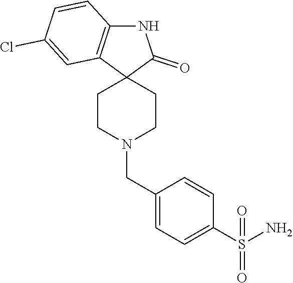 [SMILES: NS(=O)(=O)c1ccc(CN2CCC3(CC2)C(=O)Nc2ccc(Cl)cc23)cc1] | LCMS m/z 406.3 [M + H]⁺ |
| 17 | 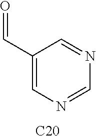 | 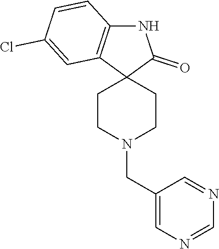 [SMILES: O=C1Nc2ccc(Cl)cc2C12CCN(Cc1cncnc1)CC2] | LCMS m/z 358.2 [M + H]⁺ |
| 18 | 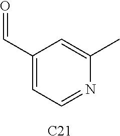 | 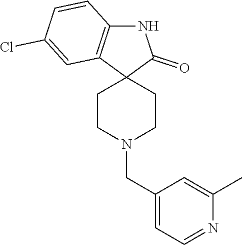 [SMILES: Cc1cc(CN2CCC3(CC2)C(=O)Nc2ccc(Cl)cc23)ccn1] | LCMS m/z 342.3 [M + H]⁺ |
| 19 | 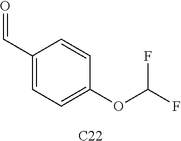 [SMILES: O=Cc1ccc(OC(F)F)cc1 ; "C22"] | 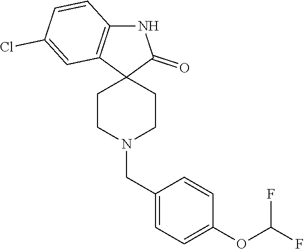 [SMILES: O=C1Nc2ccc(Cl)cc2C12CCN(Cc1ccc(OC(F)F)cc1)CC2] | LCMS m/z 393.1 [M + H]⁺ |
| 20 | 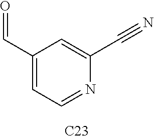 | 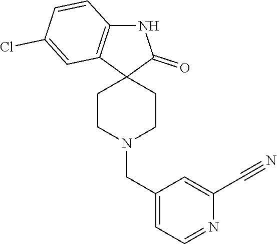 [SMILES: N#Cc1cc(CN2CCC3(CC2)C(=O)Nc2ccc(Cl)cc23)ccn1] | LCMS m/z 353.1 [M + H]⁺ |
| 21 | 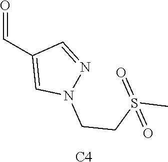 | 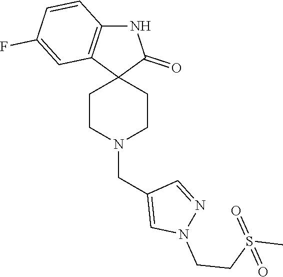 [SMILES: CS(=O)(=O)CCn1cc(CN2CCC3(CC2)C(=O)Nc2ccc(F)cc23)cn1] | ¹H NMR (300 MHz, Chloroform-d) δ 7.84 (s, 1H), 7.66 (s, 1H), 7.51 (s, 1H), 7.00 (ddd, J = 18.2, 8.5, 2.6 Hz, 2H), 6.94- 6.80 (m, 1H), 4.67 (t, J = 6.4 Hz, 2H), 4.21 (s, 1H), 3.66 (t, J = 6.4 Hz, 4H), 3.53 (d, J = 11.7 Hz, 2H), 2.75 (s, 3H), 2.63-2.45 (m, 4H), 1.96 (d, J = 14.9 Hz, 2H). LCMS m/z 407.16 [M + H]⁺ |
| 22 | 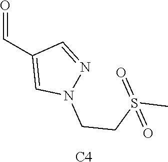 | 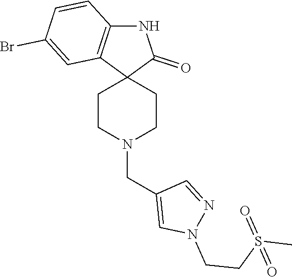 [SMILES: CS(=O)(=O)CCn1cc(CN2CCC3(CC2)C(=O)Nc2ccc(Br)cc23)cn1] | ¹H NMR (300 MHz, Chloroform-d) δ 9.10 (s, 1H), 7.70 (s, 1H), 7.59 (s, 1H), 7.46-7.28 (m, 2H), 6.79 (d, J = 8.1 Hz, 1H), 4.63 (dd, J = 6.8, 5.6 Hz, 2H), 3.90 (s, 2H), 3.66 (t, J = 6.2 Hz, 2H), 3.25 (dt, J = 11.6, 5.9 Hz, 2H), 3.13 (s, 1H), 2.61 (s, 3H), 2.23 (ddd, J = 14.8, 11.2, 4.2 Hz, 2H), 1.98-1.75 (m, 2H). LCMS m/z 467.04 [M + H]⁺ |
| 23 | 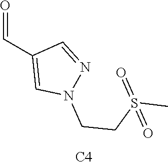 | 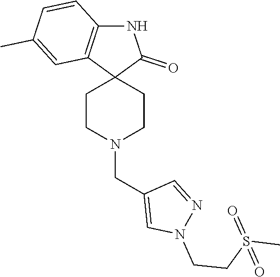 | LCMS m/z 403.09 [M + H]⁺ |
| 24 | 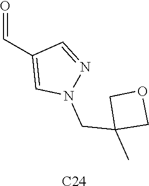 | 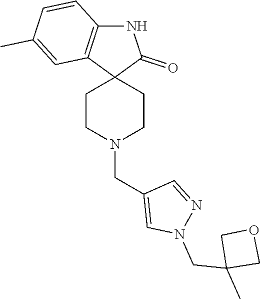 [SMILES: Cc1ccc2c(c1)C1(CCN(Cc3cnn(CC4(C)COC4)c3)CC1)C(=O)N2] | ¹H NMR (400 MHz, DMSO d6), δ = 10.2 (br s, 1H), 7.66 (s, 1H), 7.39 (s, 1H), 7.17 (s, 1H), 6.96 (d, J = 8.4 Hz, 1H), 6.71 (d, J = 7.6 Hz, 1H), 4.58 (d, J = 6.0 Hz, 2H), 4.29 (s, 2H), 4.22 (d, J = 6.0 Hz, 2H), 3.48 (s, 2H), 2.79-2.74 (m, 2H), 2.56- 2.51 (m, 2H), 2.24 (s, 3H), 1.80-1.74 (m, 2H), 1.59-1.55 (m, 2H), 1.10 (s, 3H). LCMS m/z 381.0 [M + H]⁺ |
| 25 | 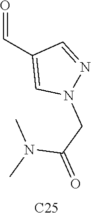 | 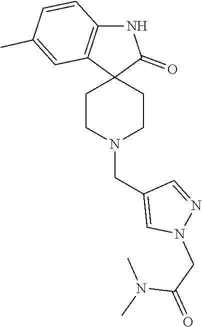 [SMILES: Cc1ccc2c(c1)C1(CCN(Cc3cnn(CC(=O)N(C)C)c3)CC1)C(=O)N2] | ¹H NMR (400 MHz, DMSO-d6) δ 10.22 (s, 1H), 7.55 (s, 1H), 7.35 (s, 1H), 7.21 (s, 1H), 6.96 (d, J = 7.6 Hz, 1H), 6.71 (d, J = 7.6 Hz, 1H), 5.04 (s, 2H) 3.48 (s, 2H), 3.01 (s, 3H), 2.86 (s, 3H), 2.81- 2.75 (m, 2H), 2.60-2.51 (m, 2H), 2.25 (s, 3H), 1.81-1.74 (m, 2H), 1.60- 1.56 (m, 2H). LCMS m/z 382.23 [M + H]⁺ |
| 26 | 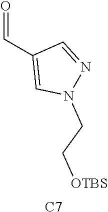 [SMILES: CC(C)(C)[Si](C)(C)OCCn1cc(C=O)cn1 ; "C7"] | 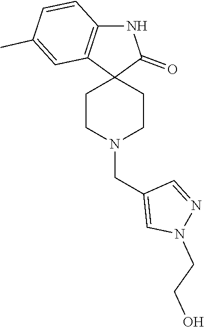 [SMILES: Cc1ccc2c(c1)C1(CCN(Cc3cnn(CCO)c3)CC1)C(=O)N2] | ¹H NMR (400 MHz, DMSO d6), δ = 10.2 (s, 1H), 7.61 (s, 1H), 7.35 (s, 1H), 7.19 (s, 1H), 6.96 (d, J = 8.8 Hz, 1H), 6.71 (d, J = 8 Hz, 1H), 4.86 (s, 1H), 4.10 (t, J = 5.6 Hz, 2H), 3.71 (d, J = 4.4 Hz, 2H), 3.46 (s, 2H), 2.80- 2.75 (m, 2H), 2.57-2.50 (m, 2H), 2.24 (s, 3H), 1.80-1.74 (m, 2H), 1.59- 1.55 (m, 2H). LCMS m/z 341.0 [M + H]⁺ |
| 27 | 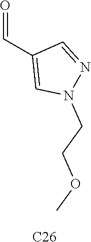 | 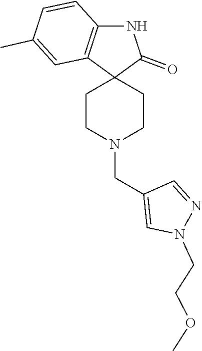 [SMILES: COCCn1cc(CN2CCC3(CC2)C(=O)Nc2ccc(C)cc23)cn1] | ¹H NMR (400 MHz, DMSO-d6) δ 10.24 (s, 1H), 8.17 (s, 1H), 7.64 (s, 1H), 7.38 (s, 1H), 7.19 (s, 1H), 6.97 (d, J = 7.6 Hz, 1H), 6.72 (d, J = 8 Hz, 1H), 4.22 (t, J = 5.2 Hz, 2H), 3.67 (t, J = 5.6 Hz, 2H) 3.53 (s, 2H), 3.22 (s, 3H), 2.87-2.79 (m, 2H), 2.67-2.61 (m, 2H), 2.25 (s, 3H), 1.81-1.76 (m, 2H), 1.63-1.55 (m, 2H). LCMS m/z 355.26 [M + H]⁺ |
| 28 | 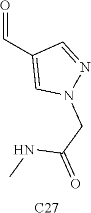 | 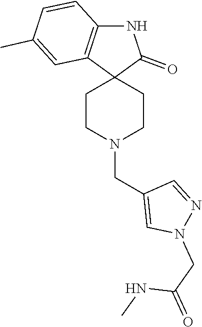 [SMILES: CNC(=O)Cn1cc(CN2CCC3(CC2)C(=O)Nc2ccc(C)cc23)cn1] | ¹H NMR (400 MHz, DMSO d6), δ = 10.2 (s, 1H), 7.93 (d, J = 4.4 Hz, 1H), 7.62 (s, 1H), 7.37 (s, 1H), 7.20 (s, 1H), 6.96 (d, J = 8.0 Hz, 1H), 6.71 (d, J = 8.0 Hz, 1H), 4.72 (s, 2H), 3.48 (s, 2H), 2.81- 2.75 (m, 2H), 2.61-2.52 (m, 5H), 2.25 (s, 3H), 1.81-1.74 (m, 2H), 1.68- 1.53 (m, 2H). LCMS m/z 368.0 [M + H]⁺ |
| 29 | 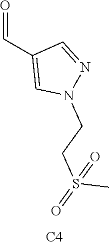 | 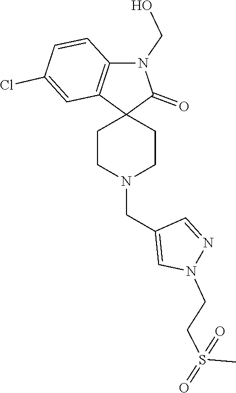 [SMILES: CS(=O)(=O)CCn1cc(CN2CCC3(CC2)C(=O)N(CO)c2ccc(Cl)cc23)cn1] | ¹H NMR (300 MHz, DMSO-d6) δ 10.43 (s, 1H), 7.75 (s, 1H), 7.44 (s, 1H), 7.31 (d, J = 2.1 Hz, 1H), 7.24 (d, J = 2.1 Hz, 1H), 5.24 (t, J = 5.7 Hz, 1H), 4.51 (t, J = 6.9 Hz, 2H), 4.45 (d, J = 5.7 Hz, 2H), 3.68 (t, J = 6.9 Hz, 2H), 3.47 (s, 2H), 2.83 (s, 3H), 2.81-2.70 (m, 2H), 2.54 (m, under DMSO, 2H), 1.83-1.61 (m, 4H). LCMS m/z 453.27 [M + H]⁺ |
| 30 | 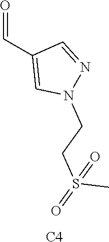 | 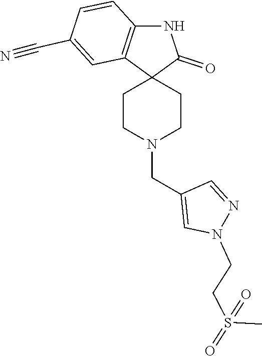 [SMILES: CS(=O)(=O)CCn1cc(CN2CCC3(CC2)C(=O)Nc2ccc(C#N)cc23)cn1] | ¹H NMR (400 MHz, Chloroform-d) δ 8.00 (s, 1H), 7.74 (d, J = 2.2 Hz, 1H), 7.65 (dd, J = 8.1, 1.7 Hz, 1H), 7.58 (d, J = 1.7 Hz, 1H), 7.11-6.98 (m, 1H), 4.70 (t, J = 6.3 Hz, 2H), 4.37 (d, J = 5.4 Hz, 2H), 3.87-3.59 (m, 6H), 3.50 (d, J = 12.3 Hz, 2H), 2.86 (s, 3H), 2.36-2.14 (m, 2H), 2.04 (dd, J = 25.0, 14.9 Hz, 3H). LCMS m/z 414.02 [M + H]⁺ |
| 31 | 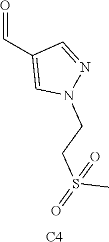 | 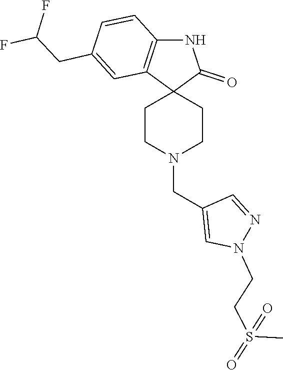 [SMILES: CS(=O)(=O)CCn1cc(CN2CCC3(CC2)C(=O)Nc2ccc(CC(F)F)cc23)cn1] | ¹H NMR (300 MHz, Chloroform-d) δ 7.46 (d, J = 17.0 Hz, 2H), 7.31 (s, 1H), 7.12 (s, 1H), 7.07- 6.97 (m, 1H), 6.75 (d, J = 7.9 Hz, 1H), 5.82 (m, 1H), 4.60-4.46 (m, 2H), 3.63-3.48 (m, 4H), 3.03 (m, 2H), 2.82 (s, 2H), 2.70-2.58 (m, 2H), 2.50 (s, 3H), 1.85 (d, J = 5.1 Hz, 4H). LCMS m/z 453.04 [M + H]⁺ |
| 32 | 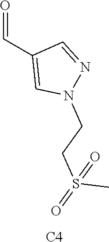 | 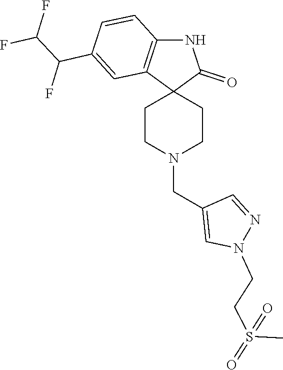 [SMILES: CS(=O)(=O)CCn1cc(CN2CCC3(CC2)C(=O)Nc2ccc(C(F)C(F)F)cc23)cn1] | ¹H NMR (400 MHz, Chloroform-d) δ 11.81 (s, 1H), 7.90 (s, 1H), 7.72- 7.64 (m, 2H), 7.37 (d, J = 8.0 Hz, 1H), 7.31 (s, 1H), 6.99 (d, J = 8.1 Hz, 1H), 6.11-5.81 (m, 1H), 5.54- 5.36 (m, 1H), 4.69 (t, J = 6.4 Hz, 2H), 4.23 (d, J = 3.5 Hz, 2H), 3.79-3.62 (m, 4H), 3.54 (d, J = 12.1 Hz, 2H), 2.80-2.74 (m, 3H), 2.71-2.57 (m, 2H), 1.98 (d, J = 15.0 Hz, 2H). LCMS m/z 471.05 [M + H]⁺ |
| 33 | 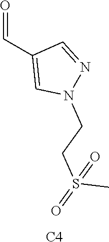 | 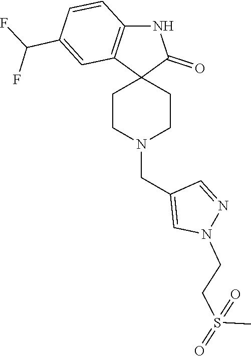 [SMILES: CS(=O)(=O)CCn1cc(CN2CCC3(CC2)C(=O)Nc2ccc(C(F)F)cc23)cn1] | ¹H NMR (400 MHz, Chloroform-d) δ 11.75 (s, 1H), 7.89 (s, 1H), 7.79 (s, 1H), 7.68 (d, J = 0.7 Hz, 1H), 7.48 (dd, J = 8.2, 1.4 Hz, 1H), 7.40 (s, 1H), 7.00 (d, J = 8.1 Hz, 1H), 6.63 (t, J = 56.3 Hz, 1H), 4.69 (dd, J = 6.8, 5.8 Hz, 2H), 4.24 (d, J = 3.4 Hz, 2H), 3.79-3.62 (m, 4H), 3.56 (d, J = 11.9 Hz, 2H), 2.77 (s, 3H), 2.64 (td, J = 14.3, 13.7, 4.3 Hz, 2H), 1.98 (d, J = 15.0 Hz, 2H). LCMS m/z 439.04 [M + H]⁺ |
| 34 | 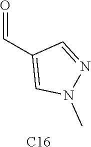 | 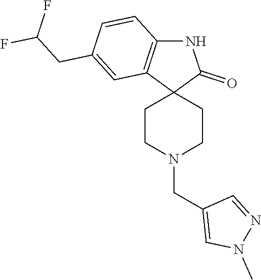 [SMILES: Cn1cc(CN2CCC3(CC2)C(=O)Nc2ccc(CC(F)F)cc23)cn1] | ¹H NMR (400 MHz, DMSO-d6) δ 10.37 (s, 1H), 7.61 (s, 1H), 7.34 (s, 2H), 7.11 (d, J = 7.9 Hz, 1H), 6.80 (d, J = 7.8 Hz, 1H), 6.37-5.97 (m, 1H), 3.81 (s, 3H), 3.47(s, 2H), 3.11 (td, J = 18.0, 4.5 Hz, 2H), 2.76 (dt, J = 15.3, 6.9 Hz, 2H), 2.55-2.45 (m, 3H), 1.83-1.52 (m, 3H). LCMS m/z 361.11 [M + H]⁺ |
| 35 | 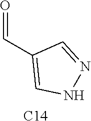 | 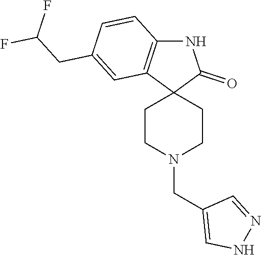 [SMILES: O=C1Nc2ccc(CC(F)F)cc2C12CCN(Cc1cn[nH]c1)CC2] | 1H NMR (400 MHz, DMSO-d6) δ 12.67 (s, 1H), 10.35 (s, 1H), 7.53 (d, J = 46.5 Hz, 1H), 7.37- 7.29 (m, 2H), 7.14 (dd, J = 33.4, 7.8 Hz, 2H), 6.80 (d, J = 7.9 Hz, 1H), 6.18 (tt, J = 56.7, 4.5 Hz, 1H), 3.50 (s, 2H), 3.22- 2.95 (m, 2H), 2.82-2.71 (m, 2H), 2.65-2.44 (m, 1H), 1.86-1.56 (m, 4H). LCMS m/z 347.42 [M + H]⁺ |
| 36 | 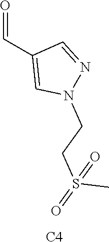 | 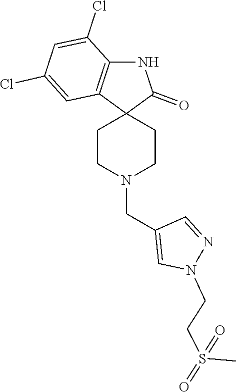 [SMILES: CS(=O)(=O)CCn1cc(CN2CCC3(CC2)C(=O)Nc2c(Cl)cc(Cl)cc23)cn1] | ¹H NMR (300 MHz, DMSO-d6) δ 10.93 (s, 1H), 7.74 (s, 1H), 7.46- 7.40 (m, 3H), 4.51 (t, J = 6.8 Hz, 2H), 3.68 (t, J = 6.9 Hz, 2H), 3.47 (s, 2H), 2.82 (s, 5H), 2.55 (d, J = 6.5 Hz, under DMSO, 2H), 1.85-1.67 (m, 4H). LCMS m/z 456.96 [M + H]⁺ |

### Compound 37

### 5-chloro-1-methyl-1′-[[1-(2-methylsulfonylethyl)pyrazol-4-yl]methyl]spiro[indoline-3,4′-piperidine]-2-one (37)

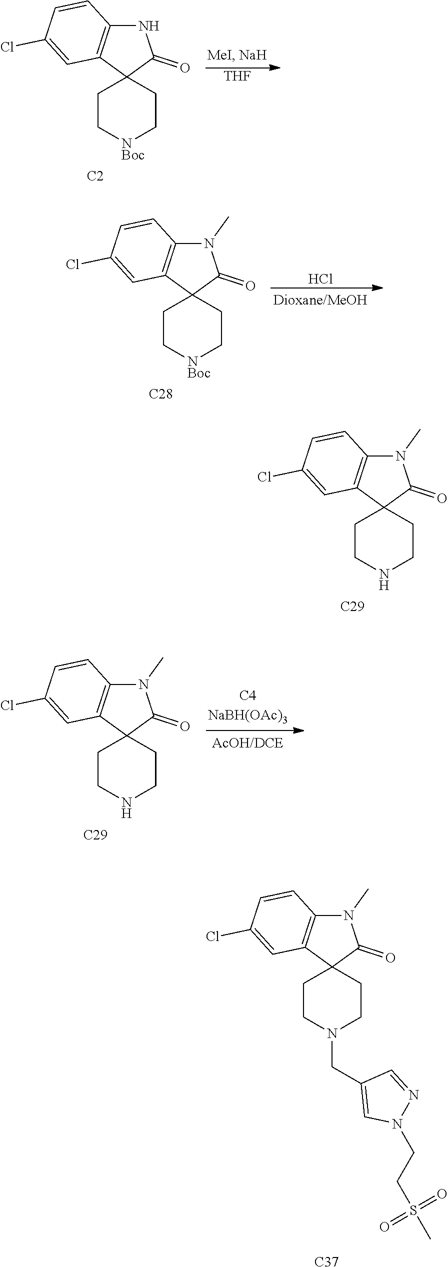

### Step 1: Preparation of tert-butyl 5-chloro-1-methyl-2-oxo-spiro[indoline-3,4′-piperidine]-1′-carboxylate (C28)

To a solution of C2 (199 mg, 0.591 mmol) and Mel (100 μL, 1.61 mmol) in 4 ml THF was added NaH (71 mg of 60% w/w, 1.78 mmol) and the mixture was stirred at rt overnight. The mixture was concentrated under vacuum and absorbed onto SiO₂ for purification by column chromatography (SiO₂, 0-50% EtOAc:heptane). The product-containing fractions were pooled and concentrated to yield the title compound C28 (199 mg, 93%) as a white solid. ¹H NMR (300 MHz, Chloroform-d) δ 7.43 (d, J=2.1 Hz, 1H), 7.32 (dd, J=8.3, 2.1 Hz, 1H), 6.98 (d, J=8.3 Hz, 1H), 3.79 (d, J=6.0 Hz, 4H), 3.19 (s, 3H), 1.84-1.68 (m, 4H), 1.50 (s, 9H). LCMS m/z 351.03 [M+H]⁺

### Step 2: Preparation of 5-chloro-1-methyl-spiro[indoline-3,4′-piperidine]-2-one (C29)

To a solution of C28 (192 mg, 0.528 mmol) in 3 ml methanol was added HCl (3 ml, 4 M in dioxane, 12 mmol) was stirred at 50° C. for 40 min. At this time the mixture was evaporated to yield the title compound C29 hydrochloride salt (156 mg, 101%) as a white solid. ¹H NMR (300 MHz, Chloroform-d) δ 7.45-7.23 (m, 2H), 7.03 (d, J=8.3 Hz, 1H), 3.82 (ddd, J=12.8, 11.9, 3.6 Hz, 2H), 3.39 (dt, J=13.0, 4.1 Hz, 2H), 3.21 (s, 3H), 2.17 (ddd, J=14.8, 12.0, 4.3 Hz, 2H), 2.06-1.90 (m, 2H). LCMS m/z 251.05 [M+H]⁺

### Step 3: Preparation of 5-chloro-1-methyl-1′-[[1-(2-methylsulfonylethyl)pyrazol-4-yl]methyl]spiro[indoline-3,4′-piperidine]-2-one (37)

To a solution of C29 (16 mg, 0.0546 mmol), C₄ (14 mg, 0.0596 mmol) and AcOH (12 μL, 0.2110 mmol)) in DCE (1 mL) was added sodium triacetoxyborohydride (36 mg, 0.171 mmol) and the mixture was stirred at rt for 6 h. At this time, the mixture was concentrated under vacuum. The material was purified by reversed-phase HPLC (C18 Waters Sunfire column (30×150 mm, 5 micron). Gradient: MeCN in H₂O with 0.2% formic acid). The product-containing fractions were pooled and concentrated to yield the title compound (37) (16.4 mg, 68%) as a clear oil. ¹H NMR (300 MHz, Chloroform-d) δ 8.51 (s, 1H), 7.79 (s, 1H), 7.62 (s, 1H), 7.29 (s, 1H), 6.85-6.70 (m, 1H), 4.65 (t, J=6.3 Hz, 2H), 4.06 (s, 2H), 3.66 (t, J=6.3 Hz, 2H), 3.50 (td, J=12.2, 3.1 Hz, 2H), 3.31 (d, J=11.7 Hz, 2H), 3.19 (s, 3H), 2.67 (s, 3H), 2.45 (td, J=13.7, 12.6, 4.2 Hz, 2H), 1.84 (d, J=14.6 Hz, 2H). LCMS m/z 437.01 [M+H]⁺

Compounds 38-40 in Table 3 were prepared in analogous fashion utilizing the listed intermediate as well as a modified intermediate that one skilled in the art of organic chemistry could access utilizing S_{N}Ar.

TABLE 3
Compounds 38-40

| | | | ¹H NMR, LCMS |
|---|---|---|---|
| Compound | Aldehyde | Structure | M + H |
| 38 | 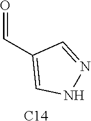 | 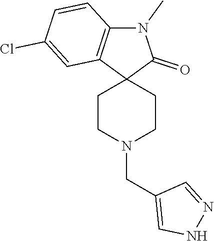 [SMILES: CN1C(=O)C2(CCN(Cc3cn[nH]c3)CC2)c2cc(Cl)ccc21] | LCMS m/z 331.04 [M + H]⁺ |
| 39 | 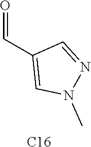 | 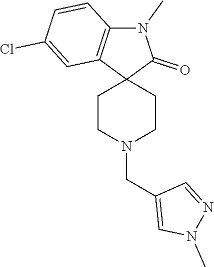 [SMILES: CN1C(=O)C2(CCN(Cc3cnn(C)c3)CC2)c2cc(Cl)ccc21] | ¹H NMR (300 MHz, Chloroform- d) δ 8.60 (s, 1H), 7.65 (s, 1H), 7.50 (s, 1H), 7.34-7.29 (m, 1H), 6.78 (d, J = 8.2 Hz, 1H), 4.04 (s, 2H), 3.93 (s, 3H), 3.48 (td, J = 12.2, 3.1 Hz, 2H), 3.39-3.27 (m, 2H), 3.19 (s, 3H), 2.45 (td, J = 13.6, 12.4, 4.3 Hz, 2H), 1.84 (d, J = 14.6 Hz, 2H). LCMS m/z 345.12 [M + H]⁺ |
| 40 | 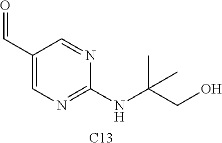 | 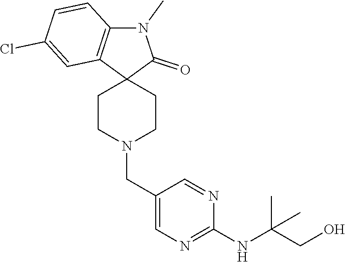 [SMILES: CN1C(=O)C2(CCN(Cc3cnc(NC(C)(C)CO)nc3)CC2)c2cc(Cl)ccc21] | ¹H NMR (300 MHz, Chloroform- d) δ 8.38 (s, 1H), 8.30 (s, 2H), 7.32 (d, J = 2.0 Hz, 1H), 7.28 (d, J = 3.3 Hz, 2H), 6.78 (d, J = 8.2 Hz, 1H), 5.65 (s, 1H), 3.70 (d, J = 4.9 Hz, 4H), 3.20 (s, 4H), 2.96 (dt, J = 10.6, 4.4 Hz, 2H), 2.11 (td, J = 12.0, 10.2, 4.0 Hz, 2H), 1.89 (d, J = 14.0 Hz, 2H), 1.40 (s, 6H). LCMS m/z 430.23 [M + H]⁺ |

### Compound 41

### 5-chloro-1′-[[1-[3-hydroxy-2-(hydroxymethyl)-2-methyl-propyl]triazol-4-yl]methyl]spiro[indoline-3,4′-piperidine]-2-one (41)

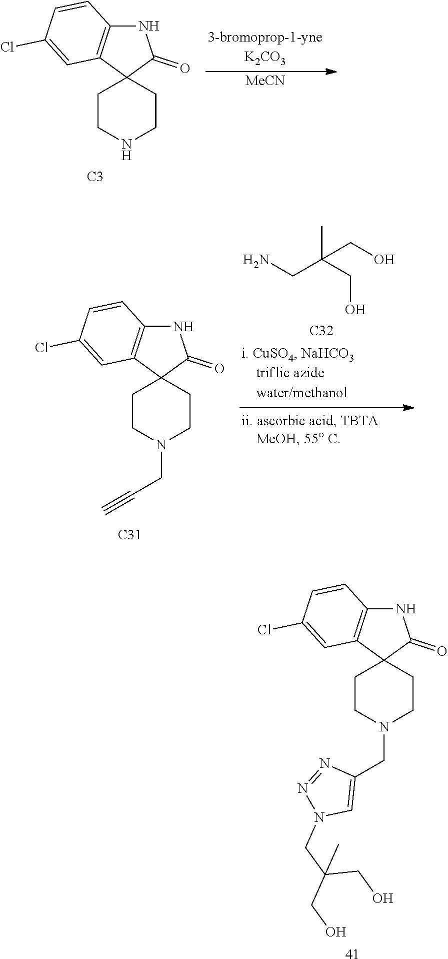

### Step 1: Preparation of 5-chloro-1′-prop-2-ynyl-spiro[indoline-3,4′-piperidine]-2-one (C31)

To a mixture of C3 hydrochloride salt (200 mg, 0.732 mmol) and potassium carbonate (200 mg, 1.45 mmol) in acetonitrile (5 mL) was added 3-bromoprop-1-yne (100 μL of 80% w/w, 0.898 mmol) and the mixture was stirred for 70 min. At this time, the mixture was diluted with DCM (20 mL) and water (10 mL). The aqueous layer was extracted with DCM (10 mL) and the organic layers were combined and passed over a phase separator. The mixture was concentrated to dryness, diluted minimally in EtOAc and purified by silica gel column chromatography (100% EtOAc). The product-containing fractions were pooled and concentrated to yield the title compound C31 (171 mg, 83%) as a clear oil. ¹H NMR (400 MHz, DMSO-d6) δ 10.53 (s, 1H), 7.51 (d, J=2.2 Hz, 1H), 7.25 (dd, J=8.3, 2.1 Hz, 1H), 6.86 (d, J=8.3 Hz, 1H), 3.39 (d, J=2.5 Hz, 2H), 3.23 (d, J=4.7 Hz, 1H), 2.90-2.80 (m, 2H), 2.70 (d, J=3.7 Hz, 2H), 1.85-1.76 (m, 2H), 1.71 (s, 2H). LCMS m/z 275.02 [M+H]⁺

### Step 2: 5-chloro-1′-[[1-[3-hydroxy-2-(hydroxymethyl)-2-methyl-propyl]triazol-4-yl]methyl]spiro[indoline-3,4′-piperidine]-2-one (41)

To a mixture of 2-(aminomethyl)-2-methyl-propane-1,3-diol (C32) (20 mg, 0.327 mmol) in methanol (3 mL) was added CuSO₄ (1 mg, 0.0063 mmol) in water (0.125 mL) followed by sodium bicarbonate (30 mg, 0.357 mmol) in water (0.5 mL) and a solution of triflic azide (0.75 mL of 0.452 M) in DCM. This mixture was stirred for 1 h. At this time, C31 (35 mg, 0.127 mmol) in methanol (0.5 mL), ascorbic acid (25 mg, 0.142 mmol) in water (0.25 mL) and TBTA (3.5 mg, 0.0066 mmol) in methanol (0.35 mL), and the mixture was heated to 55° C. and stirred for 18 h. The material was purified by reversed-phase HPLC (Waters XSelect CSH C18 OBD Prep Column; 30×150 mm, 5 micron. Gradient: Acetonitrile in Water with 0.1% Trifluoroacetic Acid). The product-containing fractions were pooled and concentrated and then rediluted in DCM (1 mL) and 1 M NaOH (1 mL) to quench residual TFA. The organic layer was passed over a phase separator and concentrated to yield the title compound 41 (10.6 mg, 20 O) as a clear oil. ¹H NMR (400 MHz, Chloroform-d) δ 7.98 (s, 1H), 7.33 (d, J=2.1 Hz, 1H), 7.21 (dd, J=8.3, 2.1 Hz, 1H), 6.86 (d, J=8.3 Hz, 1H), 4.44 (s, 2H), 3.90 (s, 2H), 3.46-3.35 (i, 4H), 3.04 (ddd, J=12.0, 8.3, 3.8 Hz, 2H), 2.80 (ddd, J=11.4, 6.6, 4.1 Hz, 2H), 2.02-1.78 (m, 4H), 0.85 (s, 3H). LCMS m/z 420.05 [M+H]z

Compounds 42-44 shown in Table 4 were prepared in analogous fashion arising from intermediate C29 in place of C3.

TABLE 4
Compounds 42-44

| Compd | Amine | Structure | ¹H NMR, LCMS M + H |
|---|---|---|---|
| 42 | 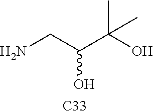 | 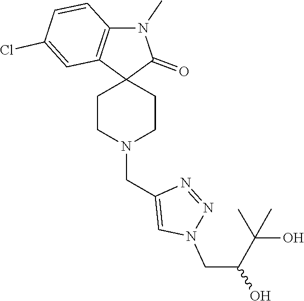 [SMILES: CN1C(=O)C2(CCN(Cc3cn(CC(O)C(C)(C)O)nn3)CC2)c2cc(Cl)ccc21] | ¹H NMR (400 MHz, Chloroform-d) δ 8.00 (s, 1H), 7.38 (d, J = 2.1 Hz, 1H), 7.30 (dd, J = 8.3, 2.1 Hz, 1H), 6.96 (d, J = 8.3 Hz, 1H), 4.78 (dd, J = 13.8, 2.2 Hz, 1H), 4.29 (dd, J = 13.9, 10.1 Hz, 1H), 3.87 (s, 2H), 3.72 (dd, J = 10.0, 2.2 Hz, 1H), 3.17 (s, 3H), 3.04 (ddd, J = 12.2, 7.7, 4.7 Hz, 2H), 2.80 (dt, J = 12.2, 5.6 Hz, 2H), 1.96-1.78 (m, 4H), 1.27 (d, J = 12.2 Hz, 6H). LCMS m/z 434.14 [M + H]⁺ |
| 43 | 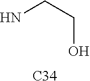 | 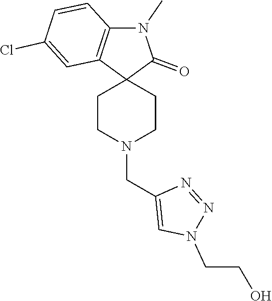 [SMILES: CN1C(=O)C2(CCN(Cc3cn(CCO)nn3)CC2)c2cc(Cl)ccc21] | ¹H NMR (400 MHz, Chloroform-d) δ 8.03 (s, 1H), 7.38 (d, J = 2.1 Hz, 1H), 7.30 (dd, J = 8.3, 2.1 Hz, 1H), 6.97 (d, J = 8.4 Hz, 1H), 4.52 (dd, J = 5.7, 4.8 Hz, 2H), 3.99-3.94 (m, 2H), 3.92 (s, 2H), 3.17 (s, 3H), 3.10 (ddd, J = 12.3, 7.0, 5.0 Hz, 2H), 2.86 (dt, J = 11.4, 5.2 Hz, 2H), 1.97- 1.79 (m, 4H). LCMS m/z 375.97 [M + H]⁺ |
| 44 | 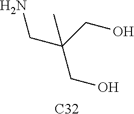 | 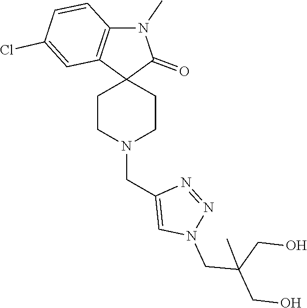 [SMILES: CN1C(=O)C2(CCN(Cc3cn(CC(C)(CO)CO)nn3)CC2)c2cc(Cl)ccc21] | ¹H NMR (400 MHz, Chloroform-d) δ 7.99 (s, 1H), 7.37 (d, J = 2.1 Hz, 1H), 7.31 (dd, J = 8.3, 2.1 Hz, 1H), 6.97 (d, J = 8.3 Hz, 1H), 4.44 (s, 2H), 3.93 (s, 2H), 3.44 (d, J = 11.1 Hz, 2H), 3.37 (d, J = 11.1 Hz, 2H), 3.17 (s, 3H), 3.08 (s, 2H), 2.88-2.79 (m, 2H), 1.90 (t, J = 5.5 Hz, 4H), 0.85 (s, 3H). LCMS m/z 434.09 [M + H]⁺ |

### Compound 45

### 5-chloro-1-(2-hydroxyethyl)-1′-[[1-(2-methylsulfonylethyl)pyrazol-4-yl]methyl]spiro[indoline-3,4′-piperidine]-2-one (45)

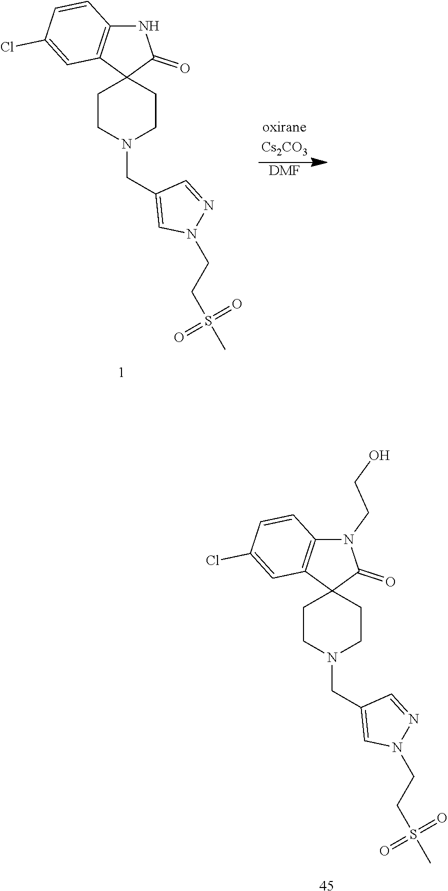

### Preparation of 5-chloro-1-(2-hydroxyethyl)-1′-[[1-(2-methylsulfonylethyl)pyrazol-4-yl]methyl]spiro[indoline-3,4′-piperidine]-2-one (45)

A suspension of 1 dihydrochloride salt (36 mg, 0.0713 mmol), Cs₂CO₃ (140 mg, 0.430 mmol), a THF solution of oxirane (1000 μL of 3 M, 3.00 mmol), and Cs₂CO₃ (140 mg, 0.430 mmol) in DMF (2 mL) was stirred at 75° C. for 16h. At this time, DCM (10 mL) was added, and the suspension was filtered, and the filtrate was evaporated to remove volatiles. The crude residue was dissolved in DMSO and the material was purified by reversed-phase HPLC (C18 Waters Sunfire column (30×150 mm, 5 micron). Gradient: MeCN in H₂O with 0.2% formic acid). The product-containing fractions were pooled and concentrated to yield the title compound 45 (13 mg, 38%) as a white solid. ¹H NMR (300 MHz, Chloroform-d) δ 8.44 (s, H), 7.85 (s, 1H), 7.64 (s, 1H), 7.25 (d, J=2.1 Hz, 1H), 6.90 (d, J=8.3 Hz, 1H), 4.66 (t, J=6.3 Hz, 2H), 4.10 (s, 2H), 3.88 (t, J=8.2, 4.1 Hz, 4H), 3.67 (t, J=6.3 Hz, 2H), 3.57 (td, J=12.5, 3.1 Hz, 2H), 3.36 (d, J=11.7 Hz, 2H), 2.71 (s, 3H), 2.61-2.40 (m, 2H), 1.86 (d, J=14.6 Hz, 2H). LCMS m/z 467.21 [M+H]⁺

Compounds 46-51 shown in Table 5 were prepared in analogous fashion arising from compound 1 utilizing alternate electrophiles. Note that compound 46 was isolated as a byproduct from the same reaction to generate 45.

TABLE 5
Compounds 46-51

| Compound | Electrophile | Structure | ¹H NMR, LCMS M + H |
|---|---|---|---|
| 46 | Oxirane | 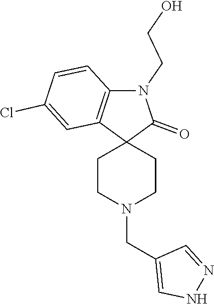 [SMILES: O=C1N(CCO)c2ccc(Cl)cc2C12CCN(Cc1cn[nH]c1)CC2] | ¹H NMR (300 MHz, Chloroform-d) δ 7.89- 7.83 (m, 2H), 7.43-7.26 (m, 2H), 7.11 (d, J = 8.3 Hz, 1H), 4.66 (t, J = 5.0 Hz, 2H), 4.42 (s, 2H), 4.22-4.03 (m, 2H), 3.78 (ddd, J = 12.6, 9.1, 3.9 Hz, 3H), 3.62-3.42 (m, 2H), 2.29 (td, J = 14.4, 13.8, 4.4 Hz, 2H), 2.02 (t, J = 16.3 Hz, 2H). LCMS m/z 360.93 [M + H]⁺ |
| 47 | 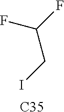 | 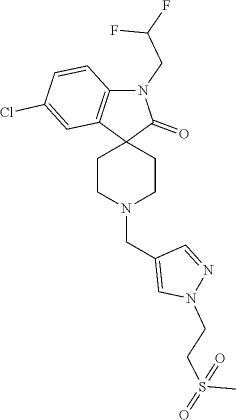 [SMILES: CS(=O)(=O)CCn1cc(CN2CCC3(CC2)C(=O)N(CC(F)F)c2ccc(Cl)cc23)cn1] | ¹H NMR (300 MHz, Chloroform-d) δ 7.57 (s, 1H), 7.52 (s, 1H), 7.32 (d, J = 2.1 Hz, 1H), 7.26 (d, J = 2.1 Hz, 1H), 6.88 (d, J = 8.3 Hz, 1H), 5.97 (tt, J = 55.5, 4.1 Hz, 1H), 4.62 (dd, J = 6.8, 5.3 Hz, 2H), 4.04 (td, J = 14.2, 4.1 Hz, 2H), 3.67 (t, J = 6.1 Hz, 2H), 3.61 (s, 2H), 2.91 (dt, J = 11.8, 6.5 Hz, 2H), 2.80-2.64 (m, 2H), 2.52 (s, 3H), 1.92 (t, J = 5.5 Hz, 4H). LCMS m/z 487.16 [M + H]⁺ |
| 48 | Ethyl iodide | 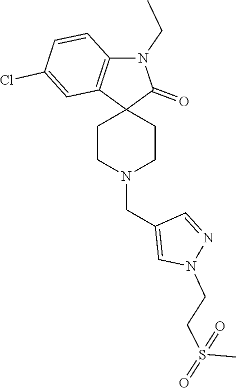 [SMILES: CCN1C(=O)C2(CCN(Cc3cnn(CCS(C)(=O)=O)c3)CC2)c2cc(Cl)ccc21] | LCMS m/z 450.99 [M + H]⁺ |
| 49 | 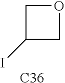 | 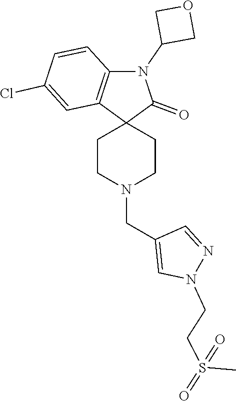 [SMILES: CS(=O)(=O)CCn1cc(CN2CCC3(CC2)C(=O)N(C2COC2)c2ccc(Cl)cc23)cn1] | LCMS m/z 479.26 [M + H]⁺ |
| 50 | 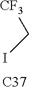 | 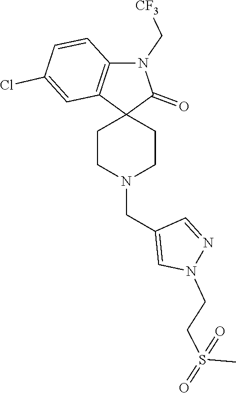 [SMILES: CS(=O)(=O)CCn1cc(CN2CCC3(CC2)C(=O)N(CC(F)(F)F)c2ccc(Cl)cc23)cn1] | LCMS m/z 504.9 [M + H]⁺ |
| 51 | 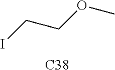 | 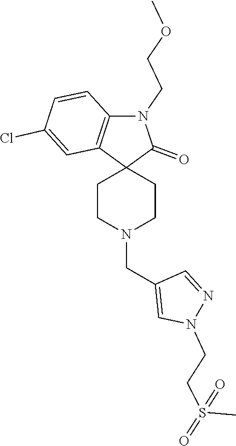 [SMILES: COCCN1C(=O)C2(CCN(Cc3cnn(CCS(C)(=O)=O)c3)CC2)c2cc(Cl)ccc21] | LCMS m/z 481.25 [M + H]⁺ |

### Compound 52

### 5-chloro-1′-[[1-(2-hydroxy-2-methyl-propyl)pyrazol-4-yl]methyl]-1-methyl-spiro[indoline-3,4′-piperidine]-2-one (52)

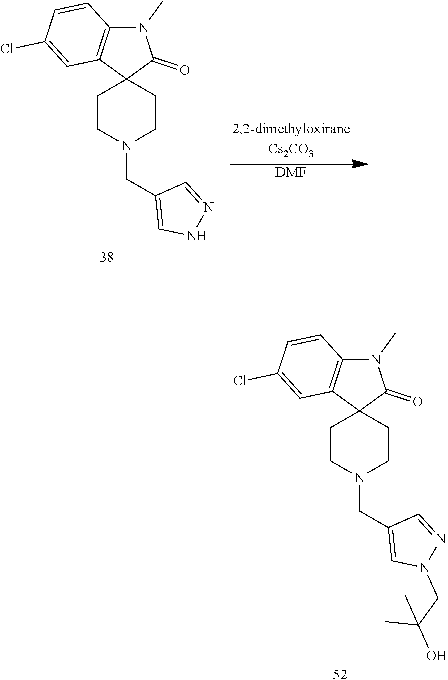

### Preparation of 5-chloro-1′-[[1-(2-hydroxy-2-methyl-propyl)pyrazol-4-yl]methyl]-1-methyl-spiro[indoline-3,4′-piperidine]-2-one (52)

A suspension of 38 (10 mg, 0.022 mmol), Cs₂CO₃ (43 mg, 0.132 mmol) and 2,2-dimethyloxirane (12 μL, 0.135 mmol) in DMF (1 mL) was stirred at 75° C. for 16 h. At this time, DCM (10 mL) was added and the mixture was filtered, concentrated under vacuum, and diluted with DMSO (1 mL). The material was purified by reversed-phase HPLC (C18 Waters Sunfire column (30×150 mm, 5 micron). Gradient: MeCN in H₂O with 0.2% formic acid). The product-containing fractions were pooled and concentrated to yield the title compound 52 (5.8 mg, 63%) as a white solid. ¹H NMR (400 MHz, Chloroform-d) δ 8.48 (s, 2H), 7.79 (d, J=0.8 Hz, 1H), 7.57 (d, J=0.7 Hz, 1H), 7.34-7.29 (m, 1H), 6.79 (dd, J=7.9, 0.8 Hz, 1H), 4.13 (2s, 3H), 3.60 (td, J=12.5, 3.1 Hz, 2H), 3.41 (d, J=12.0 Hz, 2H), 3.20 (s, 3H), 2.65 (s, 1H), 2.52 (td, J=14.0, 4.3 Hz, 2H), 1.84 (d, J=14.6 Hz, 2H), 1.21 (s, 6H). LCMS m/z 403.17 [M+H]⁺

Compounds 53-59 shown in Table 6 were prepared in analogous fashion arising from compound 38 utilizing alternative epoxides.

TABLE 6
Compounds 53-59

| Compound | Epoxide | Structure | ¹H NMR, LCMS M + H |
|---|---|---|---|
| 53 | 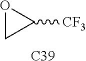 | 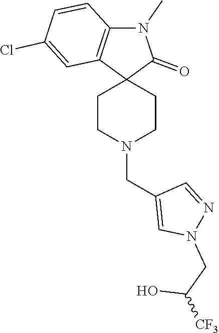 [SMILES: CN1C(=O)C2(CCN(Cc3cnn(CC(O)C(F)(F)F)c3)CC2)c2cc(Cl)ccc21] | LCMS m/z 443.11 [M + H]⁺ |
| 54 | 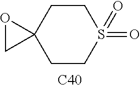 | 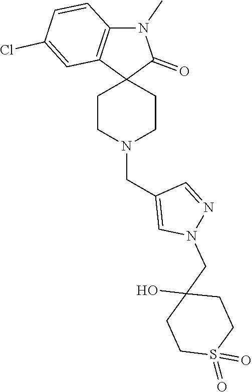 [SMILES: CN1C(=O)C2(CCN(Cc3cnn(CC4(O)CCS(=O)(=O)CC4)c3)CC2)c2cc(Cl)ccc21] | LCMS m/z 493.13 [M + H]⁺ |
| 55 | Oxirane | 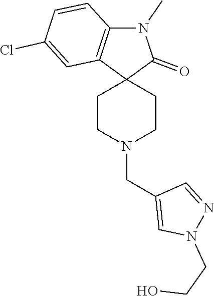 [SMILES: CN1C(=O)C2(CCN(Cc3cnn(CCO)c3)CC2)c2cc(Cl)ccc21] | ¹H NMR (300 MHz, Chloroform-d) δ 8.50 (s, 2H), 7.75 (s, 1H), 7.56 (s, 1H), 7.28 (s, 2H), 6.79 (d, J = 8.2 Hz, 1H), 4.36-4.23 (m, 2H), 4.08 (s, 2H), 4.02-3.89 (m, 2H), 3.55 (td, J = 12.2, 3.2 Hz, 2H), 3.38 (d, J = 12.0 Hz, 2H), 3.20 (s, 3H), 2.57-2.29 (m, 2H), 1.84 (d, J = 14.6 Hz, 2H). LCMS m/z 375.19 [M + H]⁺ |
| 56 | 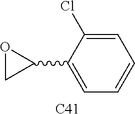 | 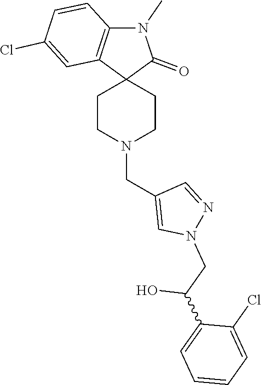 | ¹H NMR (300 MHz, Chloroform-d) δ 8.67 (s, 1H), 7.90-7.71 (m, 1H), 7.43-7.31 (m, 4H), 7.30 (s, 1H), 6.81 (d, J = 8.3 Hz, 1H), 5.45 (dd, J = 10.4, 2.7 Hz, 1H), 3.60 (td, J = 11.5, 3.3 Hz, 1H), 3.47 (d, J = 10.4 Hz, 2H), 3.22 (s, 3H), 3.20-2.97 (m, 1H), 2.83 (dd, J = 12.8, 10.4 Hz, 1H), 2.35 (ddd, J = 19.8, 14.8, 8.0 Hz, 2H), 1.95 (dd, J = 19.3, 14.8 Hz, 3H). LCMS m/z 405.32 [M + H]⁺ |
| 57 | 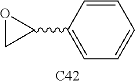 | 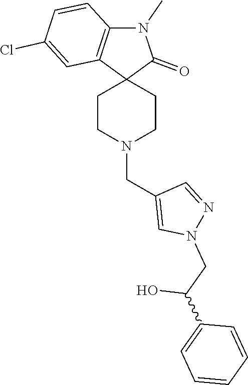 | ¹H NMR (300 MHz, Chloroform-d) δ 8.34 (s, 1H), 7.60 (ddd, J = 5.2, 3.9, 2.1 Hz, 1H), 7.55- 7.46 (m, 3H), 7.43-7.29 (m, 3H), 7.12-6.95 (m, 1H), 5.19 (dd, J = 10.0, 4.0 Hz, 1H), 4.26-4.03 (m, 1H), 3.91 (tt, J = 12.1, 3.3 Hz, 1H), 3.83- 3.46 (m, 2H), 3.46-3.36 (m, 1H), 3.18 (2s, 3H), 2.48-1.86 (m, 4H). LCMS m/z 371.17 [M + H]⁺ |
| 58 | 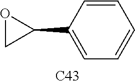 | 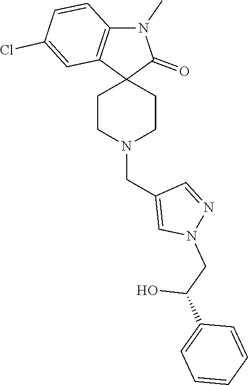 | LCMS m/z 371.17 [M + H]⁺ |
| 59 | 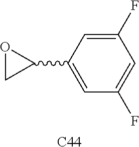 [SMILES: Fc1cc(F)cc(C2CO2)c1 ; "C44"] | 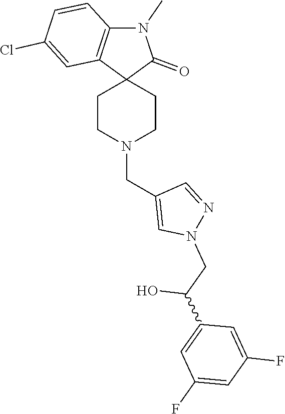 | ¹H NMR (300 MHz, Chloroform-d) δ 8.48 (s, 1H), 7.84-7.61 (m, 1H), 7.41-7.28 (m, 2H), 6.96 (t, J = 8.7 Hz, 1H), 6.88- 6.70 (m, 2H), 5.47 (t, J = 3H), 3.15 (d, J = 6.5 Hz, 6.8 Hz, 1H), 3.83-3.64 12.1 Hz, 1H), 3.22 (s, (m, 3H), 3.46 (d, J = 2H), 2.55 (dd, J = 15.7, 10.9 Hz, 2H), 2.10-1.72 (m, 2H). LCMS m/z 407.11 [M + H]⁺ |

### Compound 60

### N-[3-[(5-chloro-1-methyl-2-oxo-spiro[indoline-3,4′-piperidine]-1′-yl)methyl]cyclobutyl]-3-cyclopropyl-isoxazole-5-carboxamide (60)

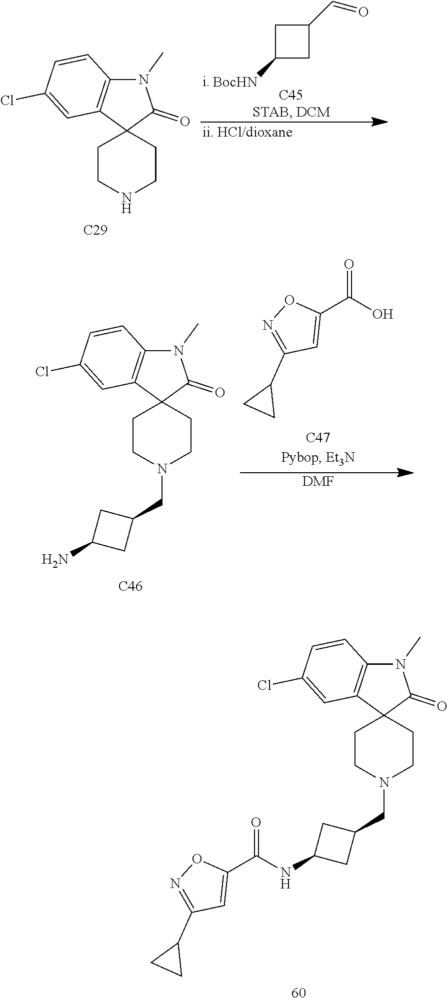

### Step 1: Preparation of 1′-[(3-aminocyclobutyl)methyl]-5-chloro-1-methyl-spiro[indoline-3,4′-piperidine]-2-one (C46)

To a solution of tert-butyl N-(3-formylcyclobutyl)carbamate (C45) (201 mg, 1.01 mmol) and C29 (207 mg, 0.721 mmol) in DCM (5 mL) was added sodium triacetoxyborohydride (460 mg, 2.17 mmol). The mixture was stirred at rt for 2 h. At this time, sat. aq. Sodium bicarbonate (5 mL) was added to the reaction mixture followed by extraction with DCM (3×5 mL). Combined organic fractions were washed with H₂O (1×2 mL), brine (1×2 mL), dried over sodium sulfate, filtered, and concentrated under vacuum.

The crude material was diluted with HCl (4 mL of 4 M, 16.00 mmol) in dioxane and stirred for 1 h. At this time, the solvent was removed under vacuum and the material was isolated as is. The title compound (C46) trifluoroacetic acid salt was isolated as a yellow oil. LCMS m/z 334.15 [M+H]⁺

### Step 2: Preparation of N-[3-[(5-chloro-1-methyl-2-oxo-spiro[indoline-3,4′-piperidine]-1′-yl)methyl]cyclobutyl]-3-cyclopropyl-isoxazole-5-carboxamide (60)

To a mixture of C46 (20 mg, 0.057 mmol) and 3-cyclopropylisoxazole-5-carboxylic acid (C47) (8.7 mg, 0.057 mmol) in DMF (1 mL) was added Et₃N (24 μL, 0.171 mmol) and PyBop (35.6 mg, 0.068 mmol) and the mixture was stirred at rt for 2 h. The solvent was evaporated under vacuum and diluted in DMSO (1 mL) and the material was purified by reversed-phase HPLC (Waters XSelect CSH C18 OBD Prep Column; 30×150 mm, 5 micron. Gradient: Acetonitrile in Water with 0.1% Trifluoroacetic Acid). The product-containing fractions were pooled and concentrated to yield the title compound (60) ditrifluoroacetic acid salt (7.5 mg, 22%) as a clear oil.

Compounds 61-71 shown in Table 7were prepared in analogous fashion arising from compound C46 or the trans-cyclobuyl analog, utilizing alternative acids, sulfonyl chlorides and carbamoyl chlorides.

TABLE 7
Compounds 61-71

| | | | ¹H NMR, LCMS |
|---|---|---|---|
| Compound | Electrophile | Structure | M + H |
| 61 | 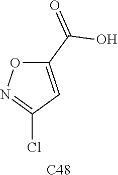 | 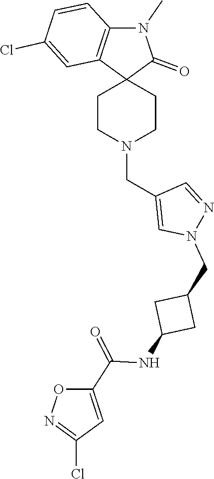 | LCMS m/z 463.06 [M + H]⁺ |
| 62 | 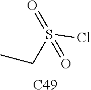 | 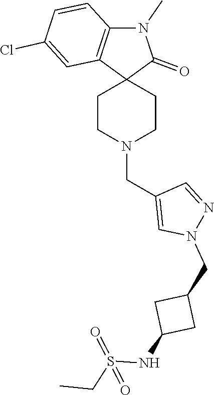 | LCMS m/z 426.13 [M + H]⁺ |
| 63 | 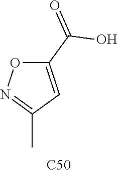 | 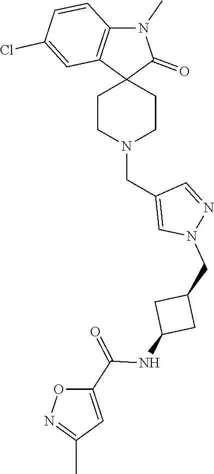 | LCMS m/z 443.15 [M + H]⁺ |
| 64 | 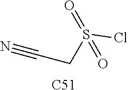 | 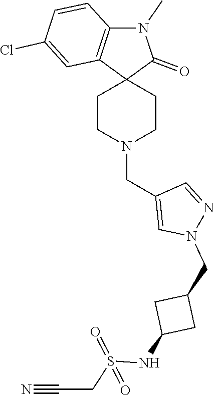 | LCMS m/z 437.1 [M + H]⁺ |
| 65 | 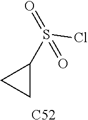 | 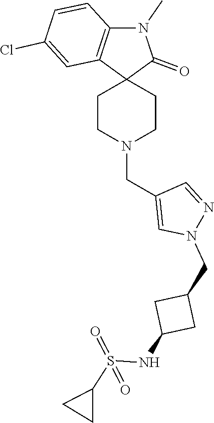 | LCMS m/z 438.14 [M + H]⁺ |
| 66 | 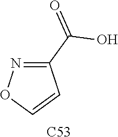 | 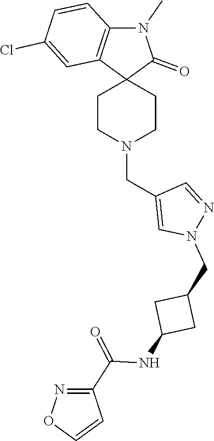 | LCMS m/z 429.15 [M + H]⁺ |
| 67 | 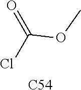 | 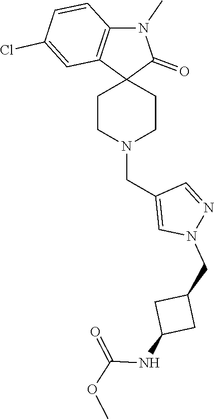 | LCMS m/z 392.17 [M + H]⁺ |
| 68 | 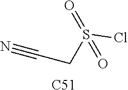 | 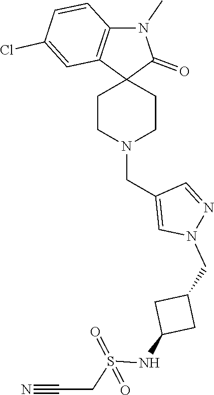 | LCMS m/z 437.1 [M + H]⁺ |
| 69 | 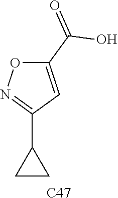 [SMILES: O=C(O)c1cc(C2CC2)no1 ; "C47"] | 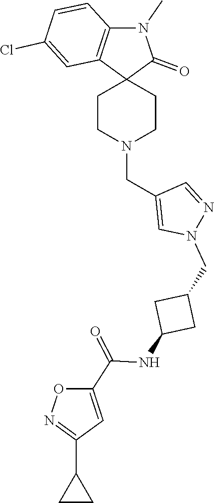 | LCMS m/z 469.16 [M + H]⁺ |
| 70 | 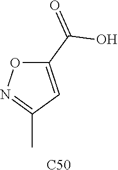 | 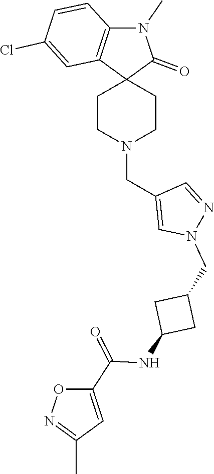 | LCMS m/z 443.15 [M + H]⁺ |
| 71 | 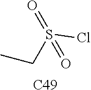 | 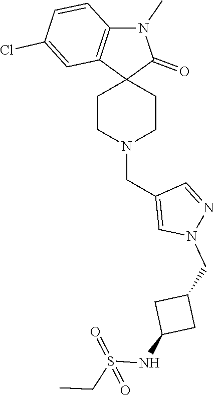 | LCMS m/z 426.17 [M + H]⁺ |

### Compound 72

### 2-(4-((I-methyl-2-oxospiro[indoline-3,4′-piperidin]-1′-yl)methyl)-1H-pyrazol-1-yl)ethane-1-sulfonamide (72)

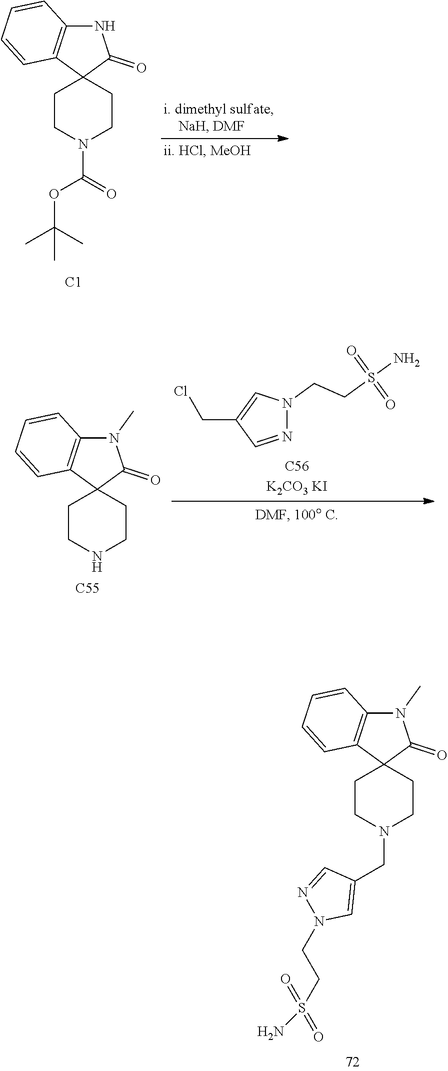

### Step 1: Preparation of 1-methylspiro[indoline-3,4′-piperidine]-2-one (C55)

A stirred solution of NaH (105.3 mg, 57% w/w, 0.0025 mol) in Toluene (20 mL) was stirred at 120° C. for 15 min. To the reaction mixture was added C1 (500 mg, 0.0017 mol) at 120° C. and stirred for 1 h at this temperature. A solution of dimethyl sulfate (252 mg, 0.19 mL, 0.0020 mol) in Toluene (1 mL) was added to the reaction at 120° C. The reaction was stirred at 120° C. for 16 h. At this time, the reaction was cooled to 0° C., quenched with water (10 mL) and extracted with EtOAc (2×50 mL). The combined organic layer dried over sodium sulfate, filtered and evaporated under vacuum to obtain a crude residue (600 mg). The crude residue was purified by column chromatography using (SiO₂, 20% EtOAc:petroleum ether). The product containing fractions were pooled and concentrated.

The residue was dissolved in 1,4-dioxane (5 mL) and to the solution was added HCl in 1,4-dioxane (1.625 mL of 4 M, 0.0065 mol) at 0° C. The reaction mass allowed to stir for 5 h. At this time, the reaction was evaporated under vacuum to get the crude residue. The crude residue was washed with diethyl ether (3×5 mL) to afford the title compound (C55) hydrochloride salt. (150 mg, 89%) as a white solid. ¹H NMR (400 MHz, DMSO-d6) δ: 8.70 (brs, 1H), 7.37-7.32 (m, 2H), 7.13-7.05 (m, 2H), 3.56-3.47 (m, 2H), 3.32-3.25 (m, 2H), 3.14 (s, 3H), 2.11-2.02 (m, 2H), 1.91-1.86 (m, 2H) LCMS m/z 217.31 [M+H]⁺

### Step 2. 2-(4-((I-methyl-2-oxospiro[indoline-3,4′-piperidin]-1′-yl)methyl)-1H-pyrazol-1-yl)ethane-1-sulfonamide (72)

To a stirred solution of C55 hydrochloride salt (75 mg, 287 μmol) in DMF (5 mL) were added K₂CO₃ (594 mg, 4.2 mmol), KI (9.5 mg, 56.8 μmol) and 2-[4-(chloromethyl)pyrazol-1-yl]ethanesulfonamide (C56) hydrochloride salt (200 mg, 384 μmol) at room temperature. The reaction mixture was stirred at 100° C. for 16 h. At this time, the reaction was filtered and washed with methanol (15 mL). The filtrate was evaporated under vacuum to get crude compound (200 mg) as brown color gum. The crude compound was purified by preparative HPLC (Gradient: 0-98% MeCN in 0.1% aqueous TFA) to provide the title compound (72) trifluoroacetic acid salt (15 mg, 10%) as a pale brown gum. VT NMR at 90° C. (400 MHz, DMSO-d6) δ: 9.74 (brs, 1H), 7.88 (brs, 1H), 7.57 (brs, 1H), 7.30 (t, J=8.4 Hz, 2H), 7.06 (t, J=7.2 Hz, 1H), 7.00 (d, J=7.6 Hz, 1H), 6.76 (s, 2H), 4.52 (t, J=7.2 Hz, 2H), 4.17 (brs, 2H), 3.52 (t, J=7.2 Hz, 2H), 3.35 (brs, 2H), 3.12 (s, 5H), 1.91-1.89 (brs, 4H), LCMS m/z 404.09 [M+H]⁺

Compound 73 was prepared in similar fashion to 72, utilizing 1-iodo-2,2-difluoroethane in step 1 and 4-(chloromethyl)-1-(2-(methylsulfonyl)ethyl)-1H-pyrazole in step 2.

TABLE 8
Compound 73

| | Electrophile | Electrophile | | ¹H NMR, LCMS |
|---|---|---|---|---|
| Compound | (step 1) | (step 2) | Structure | M + H |
| 73 | 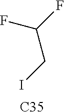 | 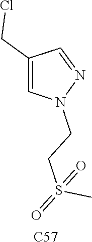 | 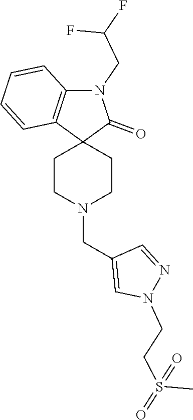 [SMILES: CS(=O)(=O)CCn1cc(CN2CCC3(CC2)C(=O)N(CC(F)F)c2ccccc23)cn1] | ¹H NMR (400 MHz, DMSO-d6) δ, 7.97 (s, 1H), 7.66 (s, 1H), 7.34-7.09 (m, 4H), 6.24 (tt, J = 54.8, 3.6 Hz, 1H), 4.59 (t, J = 6.8 Hz, 2H), 4.27-4.09 (m, 4H), 3.67 (t, J = 6.8 Hz, 2H), 3.52-3.32 (m, 4H), 2.88 (s, 3H), 2.30-1.94 (m, 4H). LCMS m/z 453.1 [M + H]⁺ |

### Compound 74

### (2′S, 3R)-5-chloro-2′-methyl-1′-[[1-(2-methylsulfonylethyl)pyrazol-4-yl]methyl]spiro[indoline-3,4′-piperidine]-2-one (74)

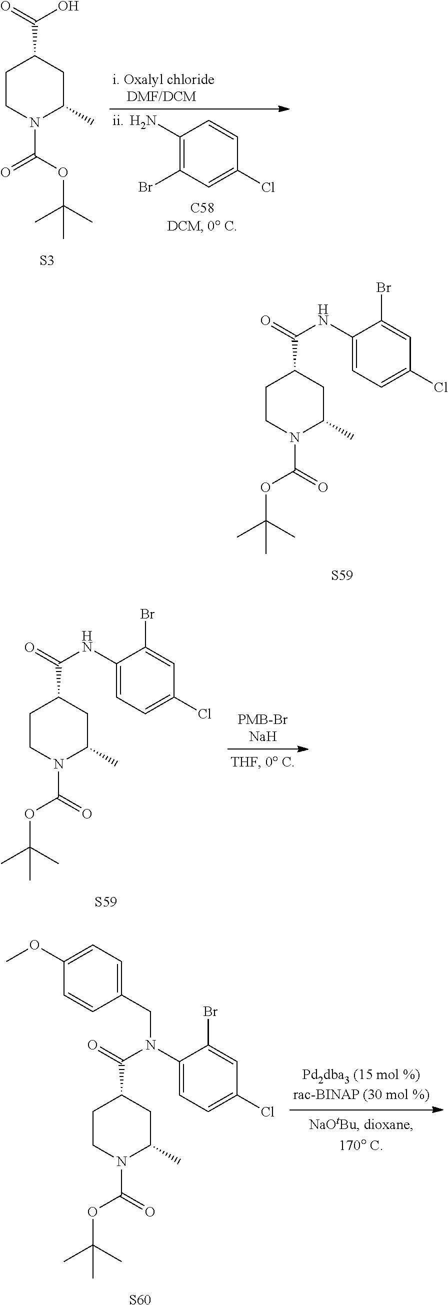

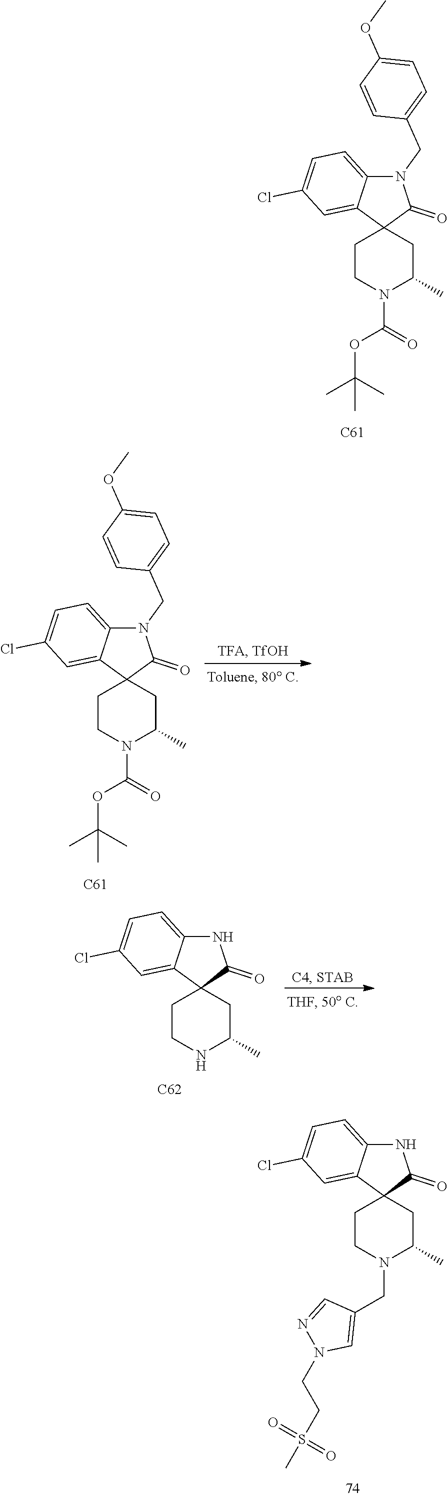

### Step 1: Preparation of tert-butyl (2S,4S)-4-[(2-bromo-4-chloro-phenyl)carbamoyl]-2-methyl-piperidine-1-carboxylate (C59)

To a suspension of (2S,4S)-1-tert-butoxycarbonyl-2-methyl-piperidine-4-carboxylic acid (S3) (104 mg, 0.428 mmol), 2-bromo-4-chloro-aniline (96 mg, 0.465 mmol) and pyridine (100 μL, 1.24 mmol) in EtOAc (1.3 mL) was added a solution of T3P (50 wt % in EtOAc, 500 μL, 0.841 mmol) and the mixture was stirred for 3.5 h. At this time, the mixture was quenched with sat. aq. sodium bicarbonate (3 mL) and extracted with EtOAc (3×3 mL). The combined organic layer was concentrated in the presence of silica gel and chromatographed (silica gel, 0-50% EtOAc:heptane). The product containing fractions were pooled and concentrated to yield the title compound (C59) (153 mg, 81%) as a white solid. LCMS m/z 431.0 [M+H]⁺

### Step 2: Preparation of tert-butyl (2S,4S)-4-[(2-bromo-4-chloro-phenyl)-[(4-methoxyphenyl)methyl]carbamoyl]-2-methyl-piperidine-1-carboxylate (C60)

To a solution of C59 (1.03 g, 2.36 mmol) in THF (12 mL) was added NaH (approximately 284 mg, 7.09 mmol, 60 wt % in mineral oil) at 0° C.

After stirring for 10 min, PMB-Br (600 μL, 4.12 mmol) was added, and after stirring for an additional 10 min the reaction was warmed to rt and stirred for 18 h. At this time, the reaction was quenched with sat. aq. sodium bicarbonate (10 mL) and extracted with DCM (3×10 mL). The combined organic layer was dried over Na₂SO₄, filtered and concentrated. The crude material was absorb onto SiO₂ and loaded onto a silica gel column for purification (0-50% EtOAc in heptane). The product-containing fractions were pooled and concentrated, and then resubjected to the purification an additional time to obtain the title compound (C60) (797 mg, 60%) as a white solid. ¹H NMR (300 MHz, CDCl₃) δ 7.70 (d, J=2.3 Hz, 1H), 7.20-7.12 (m, 1H), 7.06 (dd, J=8.7, 2.1 Hz, 2H), 6.78 (dd, J=8.7, 2.1 Hz, 2H), 6.60 (dd, J=12.4, 8.4 Hz, 1H), 5.55 (dd, J=14.2, 7.6 Hz, 1H), 3.87 (dd, J=14.2, 3.4 Hz, 1H), 3.78 (d, J=0.9 Hz, 3H), 3.63 (d, J=29.6 Hz, 2H), 3.23-2.93 (m, 1H), 2.22-1.86 (m, 1H), 1.85-1.70 (m, 1H), 1.60 (t, J=7.7 Hz, 2H), 1.38 (d, J=5.1 Hz, 9H), 1.14 (dd, J=10.0, 6.3 Hz, 3H). LCMS m/z 550.93 [M+H]⁺

### Step 3: Preparation of tert-butyl (2′S)-5-chloro-1-[(4-methoxyphenyl)methyl]-2′-methyl-2-oxo-spiro[indoline-3,4′-piperidine]-1′-carboxylate (C61)

A mixture of C60 (200 mg, 0.36 mmol), racemic BINAP Pd G3 (36 mg, 0.0363 mmol) and sodium t-butoxide (104 mg, 1.08 mmol) was purged with N2 for 15 min, at which time dioxane (3.6 mL) was added and the mixture was heated to 100° C. and stirred for 16 h. At this time, the mixture was quenched with saturated NaHCO₃ (5 mL) and the solution and extracted with EtOAc (4×5 mL). The combined organic layer was dried over Na₂SO₄, filtered, and concentrated. The crude material was purified silica gel column chromatography (0-30% EtOAc in heptane to provide product as 1.8:1 dr. The title compound (C61) (153 mg, 88%) was isolated as a clear oil. LCMS m/z 471.18 [M+H]⁺

### Step 4: Preparation of (2′S, 3R)-5-chloro-2′-methylspiro[indoline-3,4′-piperidin]-2-one (C62)

A solution of C61 (37 mg, 0.076 mmol) in toluene (300 μL) was added TFA (120 μL, 1.56 mmol). The reaction turned into an orange solution. The reaction was stirred at rt for 1 h, and then heated to 80° C. for 1 h. At this time, triflic acid (34 μL, 0.384 mmol) was added and the mixture was stirred for 18 h. At this time, the reaction was cooled to rt and quenched with sat. aq. sodium bicarbonate (2 mL) and extracted with DCM (4×2 mL). The combined organic layer was dried over Na₂SO₄, filtered, and concentrated. The crude material was purified by silica gel column chromatography (0-20% MeOH:DCM) and the product-containing fractions were pooled and resubjected to the above purification. The title compound (C62) (4.5 mg, 21%) was isolated as a clear oil. ¹H NMR (300 MHz, CDCl₃) δ 7.37 (s, 1H), 7.29 (d, J=2.1 Hz, 1H), 6.95-6.82 (m, 1H), 3.71 (ddd, J=12.4, 10.3, 3.0 Hz, 2H), 3.14 (ddd, J=12.4, 4.7, 2.1 Hz, 1H), 2.01 (td, J=13.2, 12.7, 4.6 Hz, 2H), 1.87 (dp, J=13.7, 2.1 Hz, 2H), 1.65 (dd, J=13.7, 11.4 Hz, 1H), 1.22 (d, J=6.3 Hz, 3H). LCMS m/z 251.11 [M+H]⁺

### Step 5: Preparation of (2′S,3R)-5-chloro-2′-methyl-1′-[[1-(2-methylsulfonylethyl)pyrazol-4-yl]methyl]spiro[indoline-3,4′-piperidine]-2-one (74)

To a solution of C62 (4.5 mg, 0.017 mmol), C4 (5.1 mg, 0.025 mmol) in THF (200 μL) was added sodium triacetoxyborohydride (8.9 mg, 0.042 mmol) and the mixture was heated to 50° C. and stirred for 90 min. At this time, the mixture was quenched with sat. aq. sodium bicarbonate (1 mL) and extracted with DCM (8×1 mL). The combined organic layer was dried with Na₂SO₄, filtered and concentrated. The crude material was purified by silica gel column chromatography (0-100% 3:1 EtOAc/EtOH:heptane). The product-containing fractions were pooled and concentrated and the purification was repeated once more. The title compound (74) (7.1 mg, 87%) was isolated as a clear oil. LCMS m/z 437.19 [M+H]⁺

Compounds 75-76 were prepared in analogous fashion utilizing intermediate C65 with aldehydes commercial and modified intermediates that one skilled in the art of organic chemistry could access, utilizing chemical transformations such as S_{N}2, silylation and desilylation.

TABLE 9
Compounds 75-76

| Compound | Aldehyde | Structure | ¹H NMR, LCMS M + H |
|---|---|---|---|
| 75 | 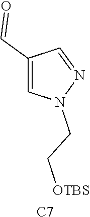 [SMILES: CC(C)(C)[Si](C)(C)OCCn1cc(C=O)cn1 ; "C7"] | 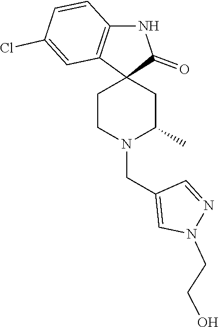 [SMILES: C[C@H]1C[C@@]2(CCN1Cc1cnn(CCO)c1)C(=O)Nc1ccc(Cl)cc12] | ¹H NMR (400 MHz, CDCl₃) δ 7.49 (s, 1H), 7.48-7.43 (m, 1H), 7.35 (s, 1H), 7.19-7.11 (m, 2H), 6.80-6.70 (m, 1H), 4.24 (dd, J = 5.7, 3.8 Hz, 2H), 4.07-3.94 (m, 2H), 3.91 (d, J = 14.5 Hz, 1H), 3.71 (d, J = 14.3 Hz, 1H), 3.17-2.94 (m, 2H), 2.76 (d, J = 11.8 Hz, 1H), 2.03 (s, 1H), 1.79-1.69 (m, 3H), 1.21 (d, J = 6.2 Hz, 3H). LCMS m/z 375.23 [M + H]⁺ |
| 76 | 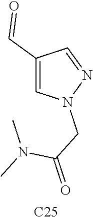 | 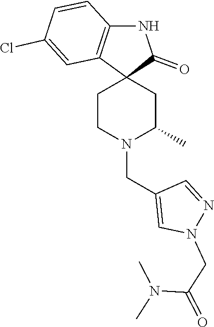 [SMILES: C[C@H]1C[C@@]2(CCN1Cc1cnn(CC(=O)N(C)C)c1)C(=O)Nc1ccc(Cl)cc12] | ¹H NMR (300 MHz, CDCl₃) δ 7.85 (s, 1H), 7.68 (s, 1H), 7.61 (s, 1H), 7.33 (d, J = 2.1 Hz, 1H), 7.21 (dd, J = 8.3, 2.1 Hz, 1H), 6.80 (d, J = 8.3 Hz, 1H), 5.14-4.92 (m, 2H), 4.60 (d, J = 14.0 Hz, 1H), 4.02 (d, J = 14.1 Hz, 2H), 3.62 (d, J = 13.5 Hz, 1H), 3.39 (d, J = 12.0 Hz, 1H), 3.10 (s, 3H), 2.99 (s, 3H), 2.85-2.45 (m, 2H), 1.95- 1.82 (m, 2H), 1.57 (d, J = 6.4 Hz, 3H). LCMS m/z 416.32 [M + H]⁺ |

### Compound 77

### 5-chloro-2′-methyl-1′-[[1-(2-methylsulfonylethyl)pyrazol-4-yl]methyl]spiro[indoline-3,4′-piperidine]-2-one trifluoroacetic acid (77)

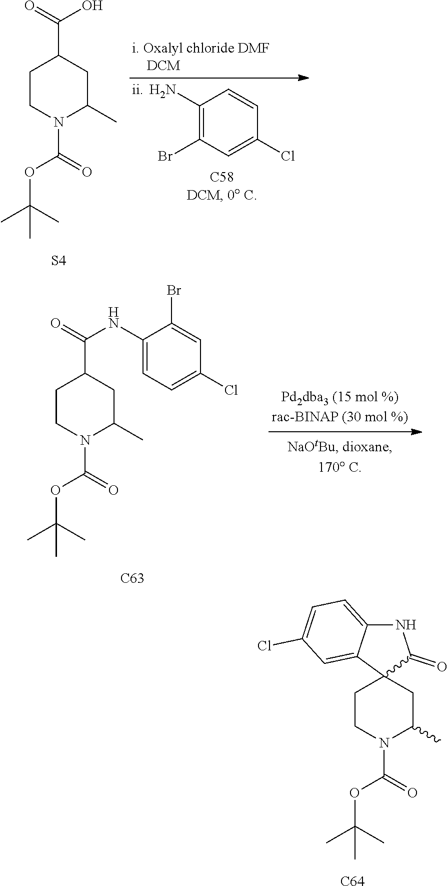

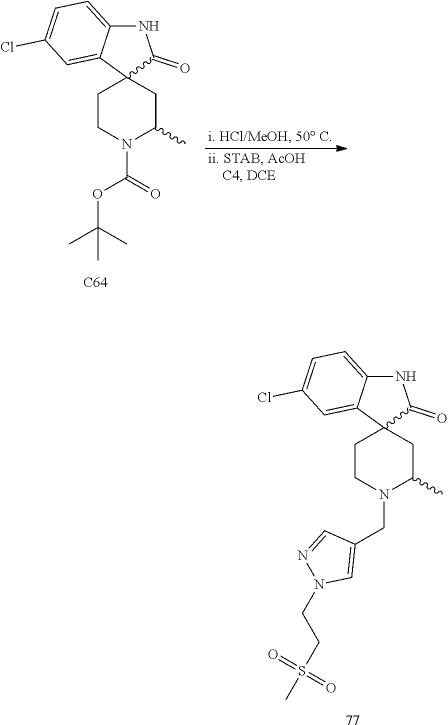

### Step 1: Preparation of tert-butyl 4-[(2-bromo-4-chloro-phenyl)carbamoyl]-2-methyl-piperidine-1-carboxylate (C63)

To 1-tert-butoxycarbonyl-2-methyl-piperidine-4-carboxylic acid (S4) (2 g, 8.22 mmol) in DCM (10 ml) was added oxalyl chloride (8.8 mL of 2 M, 17.60 mmol). At this time, a drop of DMF was added and the mixture was stirred at rt for 2 h. At this time the reaction mixture was concentrated to dryness and redissolved in pyridine (5 mL) and DCM (10 mL). The solution was cooled to 0° C. and 2-bromo-4-chloro-aniline (C58) (1.88 g, 9.11 mmol) was added. The reaction was warmed to rt and stirred for 18 h. At this time, EtOAc (300 mL) was added and the organic layer was washed with brine (300 mL), cold 1 M HCl (2×300 mL), NaHCO₃ (300 mL), and brine (300 mL). The organic layer was dried with Na₂SO₄, and concentrated with silica gel and loaded on to a silica gel column for purification (0-50% EtOAc:heptane) to yield the title compound (C63) (1.47 g, 41%) as a white solid. ¹H NMR (300 MHz, Chloroform-d) δ 8.31 (d, J=8.9 Hz, 1H), 7.68 (s, 1H), 7.56 (d, J=2.4 Hz, 1H), 7.31 (dd, J=8.9, 2.4 Hz, 1H), 4.13 (q, J=6.8 Hz, 1H), 3.98-3.79 (m, 1H), 3.28-3.04 (m, 1H), 2.61 (dtd, J=9.1, 7.3, 4.4 Hz, 1H), 2.21-1.90 (m, 3H), 1.90-1.65 (m, 1H), 1.49 (s, 9H), 1.19 (d, J=6.5 Hz, 3H). LCMS m/z 431.08 [M+H]⁺

### Step 2: Preparation of tert-butyl 5-chloro-2′-methyl-2-oxo-spiro[indoline-3,4′-piperidine]-1′-carboxylate (C64)

To a suspension of C63 (107 mg, 0.245 mmol), Pd₂dba₃ (21 mg, 0.0365 mmol) and racemic BINAP (46 mg, 0.074 mmol) in dioxane (3 mL) was added sodium tert-butoxide (71 mg, 0.74 mmol) and the reaction was heated to 170° C. for 2 h. At this time, the crude mixture was adsorbed on to silica gel and loaded on to a silica gel column for purification (0-50% EtOAc:heptane). The product-containing fractions were pooled and concentrated to yield the title compound (C64) (31 mg, 8%) as a clear oil and a 1:1 mixture of diastereomers. ¹H NMR (300 MHz, Chloroform-d) δ 9.05 (d, J=3.1 Hz, 1H), 7.20 (ddd, J=8.3, 4.2, 1.8 Hz, 1H), 7.17-6.97 (m, 1H), 6.98-6.74 (m, 1H), 4.31-3.93 (m, 1H), 3.57-3.22 (m, 1H), 2.19-1.65 (m, 5H), 1.62-1.50 (m, 9H), 1.31-1.23 (m, 5H). LCMS m/z 351.11 [M+H]⁺

### Step 3: Preparation of 5-chloro-2′-methyl-1′-[[1-(2-methylsulfonylethyl)pyrazol-4-yl]methyl]spiro[indoline-3,4′-piperidine]-2-one (77)

To a mixture of C64 in MeOH (2 mL) was added HCl in dioxane (1 mL, 4 M, 4.00 mmol) and the solution was heated to 50° C. for 1 h. At this time, the mixture was evaporated. To the crude solid was added C4 (48 mg, 0.204 mmol), DCE (4 mL), AcOH (35 μL, 0.616 mmol) and sodium triacetoxyborohydride (64 mg, 0.303 mmol) and the reaction was stirred at rt for 12 h. At this time, additional sodium triacetoxyborohydride (32 mg, 0.150 mmol) was added and the mixture was heated to 60° C. for 3 h. At this time, the mixture was concentrated to dryness, diluted in DMSO (1 mL) and the material was purified by reversed-phase HPLC (Waters XSelect CSH C18 OBD Prep Column; 30×150 mm, 5 micron. Gradient: Acetonitrile in Water with 0.1% Trifluoroacetic Acid). The product-containing fractions were pooled and concentrated to yield the title compound (77) trifluoroacetic acid salt (2 mg, 7%) as a clear oil. ¹H NMR (300 MHz, Chloroform-d) δ 8.32 (s, 1H), 7.91 (d, J=13.0 Hz, 1H), 7.73 (s, 1H), 6.97-6.89 (m, 1H), 6.79 (s, 1H), 4.81 (s, 1H), 4.68 (s, 1H), 4.48 (s, 1H), 4.23 (d, J=28.6 Hz, 1H), 3.80-3.58 (m, 2H), 3.49 (d, J=37.9 Hz, 1H), 3.23 (t, J=13.0 Hz, 1H), 2.86 (s, 2H), 1.74 (d, J=14.2 Hz, 1H), 1.65 (d, J=6.1 Hz, 3H). LCMS m/z 437.08 [M+H]⁺

Compounds 78 and 79 were prepared in analogous fashion to compound 77 using alternative anilines during step 1.

TABLE 10
Compounds 78 and 79

| Compound | Aniline | Structure | ¹H NMR, LCMS M + H |
|---|---|---|---|
| 78 | 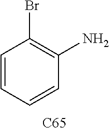 | 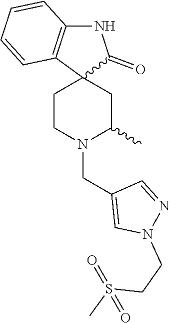 | ¹H NMR (400 MHz, CDCl₃) δ 7.49 (s, 1H), 7.48-7.43 (m, 1H), 7.35 (s, 1H), 7.19-7.11 (m, 2H), 6.80-6.70 (m, 1H), 4.24 (dd, J = 5.7, 3.8 Hz, 2H), 4.07-3.94 (m, 2H), 3.91 (d, J = 14.5 Hz, 1H), 3.71 (d, J = 14.3 Hz, 1H), 3.17-2.94 (m, 2H), 2.76 (d, J = 11.8 Hz, 1H), 2.03 (s, 1H), 1.79-1.69 (m, 3H), 1.21 (d, J = 6.2 Hz, 3H). LCMS m/z 375.23 [M + H]⁺ |
| 79 | 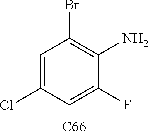 | 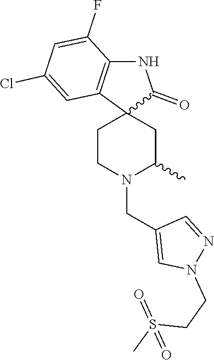 | ¹H NMR (300 MHz, CDCl₃) δ 7.85 (s, 1H), 7.68 (s, 1H), 7.61 (s, 1H), 7.33 (d, J = 2.1 Hz, 1H), 7.21 (dd, J = 8.3, 2.1 Hz, 1H), 6.80 (d, J = 8.3 Hz, 1H), 5.14-4.92 (m, 2H), 4.60 (d, J = 14.0 Hz, 1H), 4.02 (d, J = 14.1 Hz, 2H), 3.62 (d, J = 13.5 Hz, 1H), 3.39 (d, J = 12.0 Hz, 1H), 3.10 (s, 3H), 2.99 (s, 3H), 2.85- 2.45 (m, 2H), 1.95-1.82 (m, 2H), 1.57 (d, J = 6.4 Hz, 3H). LCMS m/z 416.32 [M + H]⁺ |

### Compound 80

### (2′S,3R)-5-chloro-1,2′-dimethyl-1′-[[1-(2-methylsulfonylethyl)pyrazol-4-yl]methyl]spiro[indoline-3,4′-piperidine]-2-one trifluoracetic acid (80)

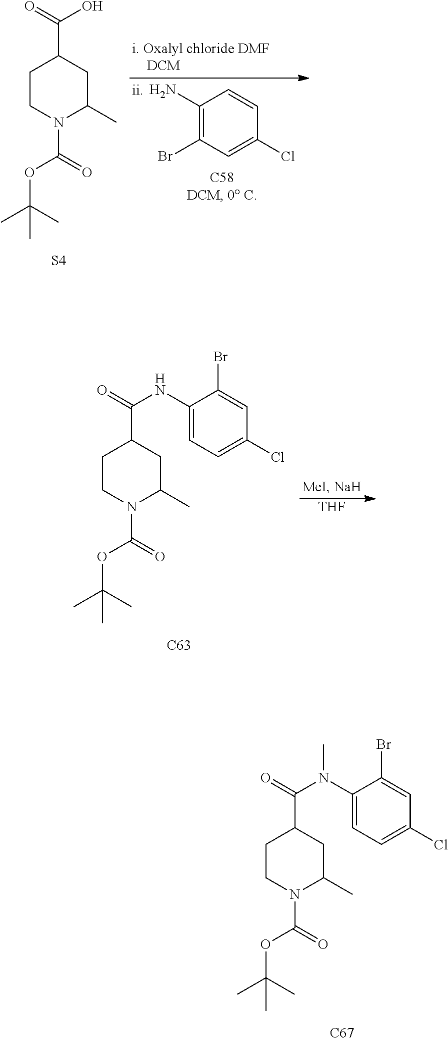

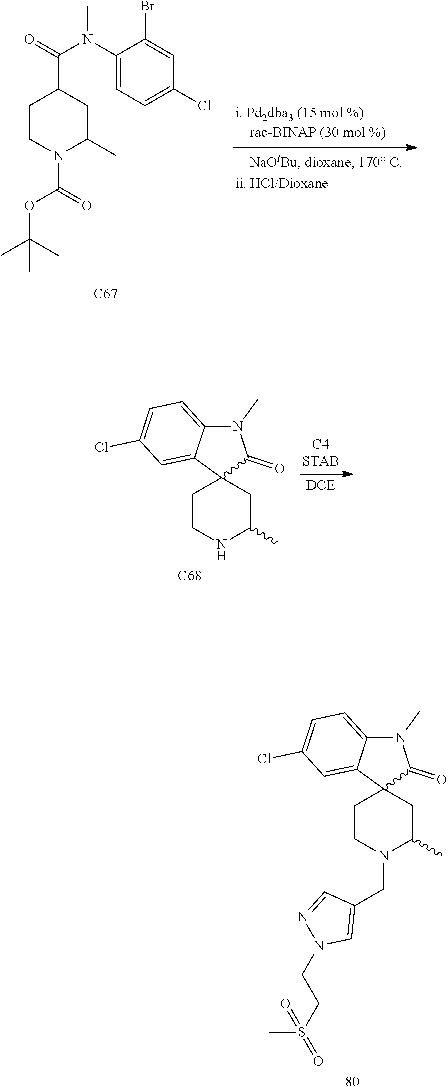

### Step 1: Preparation of tert-butyl 4-[(2-bromo-4-chloro-phenyl)carbamoyl]-2-methyl-piperidine-1-carboxylate (C63)

See procedure above for preparing intermediate C63 in the preparation of Compound 77.

### Step 2: Preparation of tert-butyl 4-[(2-bromo-4-chloro-phenyl)-methyl-carbamoyl]-2-methyl-piperidine-1-carboxylate (C67)

To a solution of C63 (310 mg, 0.711 mmol) and Mel (135 μL, 2.17 mmol) in 4 ml THF was added NaH (90 mg of 60% w/w, 2.25 mmol) and the mixture was stirred at RT overnight. At this time, the mixture was adsorbed on to silica gel and purified by silica gel column chromatography (0-50% EtOAc:heptane). The product-containing fractions were pooled and concentrated to yield the title compound (C67) (294 mg, 89%) as a yellow oil. LCMS m/z 445.08 [M+H]⁺

### Step 3: Preparation of (2′S,3R)-5-chloro-1, 2′-dimethyl-spiro[indoline-3,4′-piperidine]-2-one (C68)

To a suspension of C67 (285 mg, 0.615 mmol), Pd₂dba₃ (53 mg, 0.092 mmol) and racemic BINAP (115 mg, 0.185 mmol) in dioxane (8 mL) was added sodium tert-butoxide (120 mg, 1.25 mmol). The reaction was heated to 160° C. for 2.5 h. At this time, the reaction was concentrated and diluted with 3 ml MeOH and HCl (3 mL of 4 M, 12.00 mmol) in dioxane and the reaction was heated to 50° C. for 40 min. The mixture was concentrated and diluted in DMSO (1 mL) and loaded on to a C18 column for reversed-phase purification (10-100% MeCN:water, 0.1% TFA modifier). The product-containing fractions were concentrated, diluted in HCl dioxane to yield the HCl salt. The title compound (C68) hydrochloride salt (49.9 mg, 25%) ¹H NMR (300 MHz, Chloroform-d) δ 7.41-7.25 (m, 2H), 7.01 (dd, J=8.2, 0.5 Hz, 1H), 4.13 (h, J=6.8 Hz, 1H), 3.96-3.80 (m, 1H), 3.39 (ddd, J=12.7, 4.6, 2.0 Hz, 1H), 3.31 (p, J=1.7 Hz, 3H), 2.17 (ddd, J=14.9, 13.6, 4.6 Hz, 1H), 2.06-1.89 (m, 3H), 1.34 (d, J=6.6 Hz, 3H). LCMS m/z 265.01 [M+H]⁺ Retention time: 0.61 minutes. Final purity was determined by reversed phase UPLC using an Acquity UPLC Acquity CSH C18 (2.1×50 mm, 1.7 μm particle) made by Waters, and a dual gradient run from 5-95% mobile phase B over 0.6 minutes. Mobile phase A=H₂O (0.1% CF₃CO₂H). Mobile phase B═CH3CN (0.1% CF₃CO₂H). Flow rate=0.6 m/min, injection volume=2.0 μL.

### Step 4: Preparation of (2′S,3R)-5-chloro-1,2′-dimethyl-1′-[[1-(2-methylsulfonylethyl)pyrazol-4-yl]methyl]spiro[indoline-3,4′-piperidine]-2-one (80)

To C68 (12 mg, 0.037 mmol) in 2 ml DCE was C4 (20 mg, 0.085 mmol). After 5 min, sodium triacetoxyborohydride (28 mg, 0.133 mmol) was added and the mixture was stirred at rt for 14 h. At this time, the mixture was evaporated, diluted in DMSO (1 mL) and the material was purified by reversed-phase HPLC (Waters XSelect CSH C18 OBD Prep Column; 30×150 mm, 5 micron. Gradient: Acetonitrile in Water with 0.1% Trifluoroacetic Acid). The product-containing fractions were pooled and concentrated to yield the title compound (80) trifluoroacetic acid salt (4.9 mg, 22%) LCMS m/z 451.09 [M+H]⁺

Compounds 81 to 87 were prepared in analogous fashion to compound 80, utilizing alternative anilines in step 1 as well as an alternative piperidine.

TABLE 11
Compounds 81-87

| Compound | Aniline | Structure | ¹H NMR, LCMS M + H |
|---|---|---|---|
| 81 | 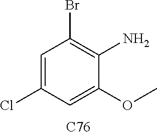 | 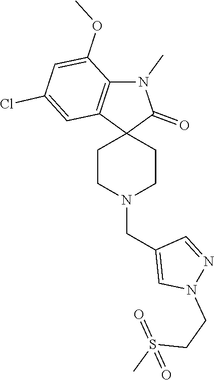 | LCMS m/z 453.04 [M + H]⁺ |
| 82 | 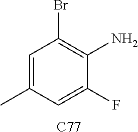 | 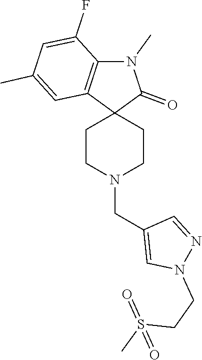 [SMILES: Cc1cc(F)c2c(c1)C1(CCN(Cc3cnn(CCS(C)(=O)=O)c3)CC1)C(=O)N2C] | ¹H NMR (500 MHz, DMSO-d6) δ 8.03 (d, J = 7.1 Hz, 1H), 7.70 (d, J = 8.3 Hz, 1H), 7.16-7.02 (m, 1H), 6.82 (s, 1H), 4.66-4.56 (m, 2H), 4.49- 4.45 (m, 1H), 4.36-4.32 (m, 1H), 3.71 (t, J = 6.7 Hz, 2H), 3.57-3.51 (m, 3H), 3.42-3.36 (m, 2H), 3.29 (dd, J = 19.8, 2.6 Hz, 4H), 2.92 (d, J = 14.0 Hz, 3H), 2.33 (d, J = 27.9 Hz, 3H), 2.22-2.16 (m, 1H), 2.14-2.04 (m, 1H), 1.96 (d, J = 14.6 Hz, 1H), 1.88 (d, J = 14.7 Hz, 1H). LCMS m/z 435.4 [M + H]⁺ |
| 83 | 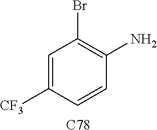 [SMILES: Nc1ccc(C(F)(F)F)cc1Br ; "C78"] | 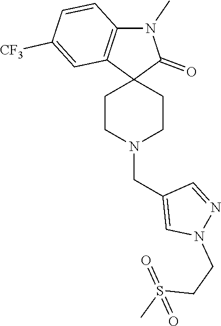 [SMILES: CN1C(=O)C2(CCN(Cc3cnn(CCS(C)(=O)=O)c3)CC2)c2cc(C(F)(F)F)ccc21] | ¹H NMR (500 MHz, DMSO-d6) δ 10.24 (s, 1H), 8.03 (d, J = 8.1 Hz, 1H), 7.69 (d, J = 16.6 Hz, 1H), 7.39 (dd, J = 30.8, 8.5 Hz, 1H), 7.24- 7.18 (m, 1H), 7.16 (d, J = 8.5 Hz, 1H), 4.66-4.55 (m, 2H), 4.54-4.47 (m, 1H), 4.38-4.30 (m, 1H), 3.71 (t, J = 6.7 Hz, 2H), 3.64-3.49 (m, 2H), 3.41 (d, J = 12.2 Hz, 1H), 3.29 (q, J = 11.4, 11.0 Hz, 1H), 3.16 (d, J = 17.7 Hz, 3H), 2.91 (d, J = 13.0 Hz, 3H), 2.37-2.24 (m, 1H), 2.17-2.04 (m, 1H), 2.03- 1.88 (m, 1H). LCMS m/z 471.4 [M + H]⁺ |
| 84 | 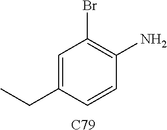 | 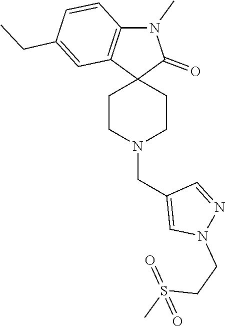 [SMILES: CCc1ccc2c(c1)C1(CCN(Cc3cnn(CCS(C)(=O)=O)c3)CC1)C(=O)N2C] | LCMS m/z 431.5 [M + H]⁺ |
| 85 | 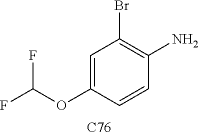 [SMILES: Nc1ccc(OC(F)F)cc1Br ; "C76"] | 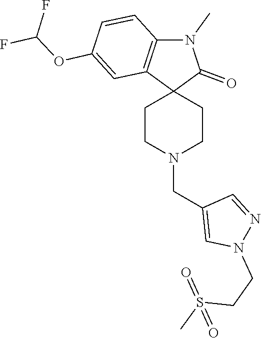 [SMILES: CN1C(=O)C2(CCN(Cc3cnn(CCS(C)(=O)=O)c3)CC2)c2cc(OC(F)F)ccc21] | ¹H NMR (500 MHz, DMSO-d6) δ 8.03 (d, J = 12.9 Hz, 1H), 7.69 (d, J = 15.6 Hz, 1H), 7.26-7.12 (m, 2H), 7.12-7.08 (m, 1H), 7.03-6.99 (m, 1H), 4.65-4.58 (m, 2H), 4.51-4.46 (m, 1H), 4.37-4.32 (m, 1H), 3.71 (t, J = 6.7 Hz, 3H), 3.60 (s, 1H), 3.43-3.37 (m, 1H), 3.30 (d, J = 11.3 Hz, 1H), 3.14 (d, J = 17.1 Hz, 3H), 2.92 (d, J = 11.2 Hz, 3H), 2.25 (td, J = 14.3, 4.3 Hz, 1H), 2.15-2.05 (m, 1H), 1.96 (d, J = 14.5 Hz, 1H), 1.89 (d, J = 14.7 Hz, 1H). LCMS m/z 469.6 [M + H]⁺ |
| 86 | 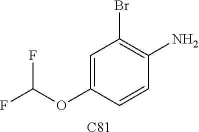 [SMILES: Nc1ccc(OC(F)F)cc1Br ; "C81"] | 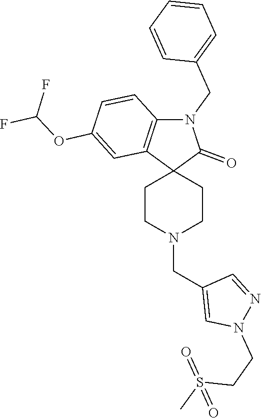 [SMILES: CS(=O)(=O)CCn1cc(CN2CCC3(CC2)C(=O)N(Cc2ccccc2)c2ccc(OC(F)F)cc23)cn1] | ¹H NMR (500 MHz, DMSO-d6) δ 9.88 (d, J = 63.5 Hz, 1H), 8.04 (d, J = 13.6 Hz, 1H), 7.70 (d, J = 17.9 Hz, 1H), 7.38-7.24 (m, 5H), 7.19- 7.06 (m, 1H), 7.06-7.01 (m, 1H), 6.96 (d, J = 8.6 Hz, 1H), 4.92 (d, J = 20.9 Hz, 2H), 4.67-4.57 (m, 2H), 4.56-4.46 (m, 1H), 4.41- 4.34 (m, 1H), 3.72 (t, J = 6.7 Hz, 2H), 3.64-3.56 (m, 2H), 3.48- 3.41 (m, 1H), 3.40-3.29 (m, 1H), 2.91 (d, J = 14.4 Hz, 3H), 2.39-2.26 (m, 1H), 2.23-2.13 (m, 1H), 2.07- 1.93 (m, 2H). LCMS m/z 545.2 [M + H]⁺ |
| 87 | 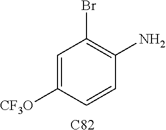 | 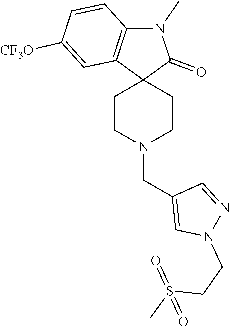 | ¹H NMR (500 MHz, DMSO-d6) δ 10.06 (s, 1H), 8.05 (s, 1H), 7.81-7.66 (m, 2H), 7.47 (s, 1H), 7.28 (dd, J = 16.1, 8.2 Hz, 1H), 4.68-4.49 (m, 3H), 4.42-4.29 (m, 1H), 3.72 (t, J = 6.7 Hz, 2H), 3.68-3.59 (m, 1H), 3.59-3.47 (m, 1H), 3.47-3.38 (m, 1H), 3.38-3.25 (m, 1H), 3.19 (d, J = 15.9 Hz, 3H), 2.91 (d, J = 11.7 Hz, 3H), 2.42-2.27 (m, 1H), 2.12-1.93 (m, 2H). LCMS m/z 487.3 [M + H]⁺ |

### Compound S1

### (1R,2′S)-7-chloro-2′-methyl-spiro[isochromane-1,4′-piperidine](S1)

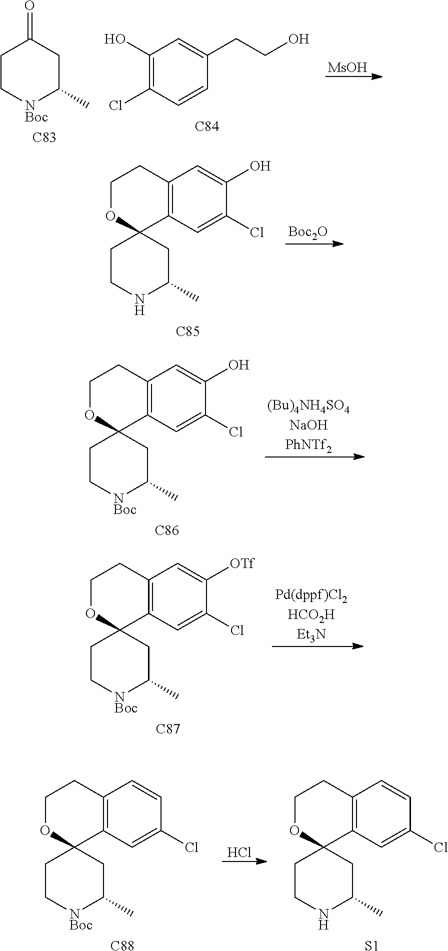

### Step 1. Synthesis of (1R,2′S)-7-chloro-2′-methyl-spiro[isochromane-1,4′-piperidine]-6-ol (C85)

A solution of tert-butyl (2S)-2-methyl-4-oxo-piperidine-1-carboxylate C83 (9.21 g, 38.00 mmol) in DCM (125 mL) was cooled with an ice bath to approximately 6° C. Methanesulfonic acid (10.8 mL, 166.4 mmol) was added in 4 portions over the course of 5 minutes. Following completion of addition, the reaction was warmed with a water bath at 35° C. and then bath removed and the reaction stirred at room temperature for 15 minutes. House vacuum was pulled for 2 minutes to remove any residual isobutylene. To this solution was added 2-chloro-5-(2-hydroxyethyl)phenol C84 (7 g, 40.55 mmol) and the reaction mixture was allowed to warm to 36° C. with the aid of a heating mantle. The reaction was stirred at stirred at this temperature under a reflux condenser for 15 hours. The reaction was warmed to ambient temperature, poured into a 1 L erlenmeyer flask, and cooled on brine/ice bath while stirring. Added 6 M NaOH slowly until pH was adjusted to 9. A white colorless precipitate formed. Continued to stir for 30 minutes while cold. Solids were filtered, air dried and then vacuumed to dryness to afford (1R,2′S)-7-chloro-2′-methyl-spiro[isochromane-1,4′-piperidine]-6-ol C85 as white solid (10.5 g, 98%) ¹H NMR (300 MHz, DMSO-d6) δ 7.09 (s, 1H), 6.75 (s, 1H), 3.81 (td, J=5.8, 2.6 Hz, 2H), 3.44-2.87 (m, 4H), 2.65 (t, J=5.6 Hz, 2H), 2.18 (dt, J=13.5, 7.2 Hz, 1H), 2.10-1.83 (m, 3H), 1.26 (d, J=6.5 Hz, 3H). LCMS m/z 267.97 [M+H]⁺.

### Step 2. Synthesis of tert-butyl (1R,2′S)-7-chloro-6-hydroxy-2′-methyl-spiro[isochromane-1,4′-piperidine]-1′-carboxylate (C86)

To a solution of (1R,2′S)-7-chloro-2′-methyl-spiro[isochromane-1,4′-piperidine]-6-ol C₈₅ (3.6 g, 12.77 mmol) in DCM (45 mL) was added with Boc₂O (2.9 g, 13.29 mmol) and then sodium bicarbonate (33 mL of 1.8 M, 59.40 mmol) and stirred at room temperature for 16 hours. The aqueous layer was extracted with DCM (50 ml) through a phase separator then back-extracted three times each time mixing with DCM (50 mL). The combined organic extracts were washed with brine, dried over Na₂SO₄, filtered, and concentrated in vacuo to afford the crude product tert-butyl (1R,2′S)-7-chloro-6-hydroxy-2′-methyl-spiro[isochromane-1,4′-piperidine]-1′-carboxylate C86 (4.8 g, 97%) LCMS m/z 268.03 [M-100+H]⁺.

### Step 2. Synthesis of tert-butyl (1R,2′S)-7-chloro-2′-methyl-6-(trifluoromethylsulfonyloxy)spiro[isochromane-1,4′-piperidine]-1′-carboxylate (C87)

To a suspension of tert-butyl (1R,2′S)-7-chloro-6-hydroxy-2′-methyl-spiro[isochromane-1,4′-piperidine]-1′-carboxylate C86 (5.7 g, 15.49 mmol), in DCM (126 mL) was added 1,1,1-trifluoro-N-phenyl-N-(trifluoromethylsulfonyl)methanesulfonamide (17 g, 47.59 mmol), tetrabutylammonium hydrogen sulfate (9 g, 26.51 mmol) and NaOH (72 mL of 2.0 M, 144.0 mmol). The flask was fitted with an air condenser, and the reaction was stirred vigorously at 30° C. for 16 hours. The aqueous layer was extracted using DCM (3×50 ml). Added 50 ml of DCM to the aqueous layer and the pH was adjusted to 8-9 using 2 M HCl. Extracted using DCM (3×50 ml). The combined organic extracts were washed with brine, dried over Na₂SO₄, filtered, and concentrated in vacuo to afford the crude material as a gummy solid. The crude was purified by silica gel chromatography using 0 to 50% ethyl acetate in heptane to afford tert-butyl (1R,2′S)-7-chloro-2′-methyl-6-(trifluoromethylsulfonyloxy)spiro[isochromane-1,4′-piperidine]-1′-carboxylate C87 (7.9 g, 97%) ¹H NMR (400 MHz, Chloroform-d) δ 7.44-7.27 (m, 2H), 7.21 (s, 1H), 7.08 (s, 1H), 4.03 (ddd, J=11.7, 6.8, 5.4 Hz, 1H), 3.94-3.74 (m, 3H), 3.38 (ddd, J=14.3, 9.4, 5.3 Hz, 1H), 2.89-2.67 (m, 2H), 2.15 (dddd, J=15.6, 9.4, 6.2, 1.7 Hz, 1H), 2.08-1.98 (m, 1H), 1.89-1.69 (m, 3H), 1.51 (s, 9H), 1.28 (d, J=6.5 Hz, 4H). LCMS m/z 400.05 [M-Boc+H]⁺.

### Step 4. Synthesis of tert-butyl (1R,2′S)-7-chloro-2′-methyl-spiro[isochromane-1,4′-piperidine]-1′-carboxylate (C88)

To a suspension of tert-butyl (1R,2′S)-7-chloro-2′-methyl-6-(trifluoromethylsulfonyloxy) spiro[isochromane-1,4′-piperidine]-1′-carboxylate C87 (375 mg, 0.7501 mmol) in DMF (7 mL) was added Pd(dppf)Cl₂ (63 mg, 0.07715 mmol), triethyl amine (360 μL, 2.583 mmol) followed by formic acid (72 μL, 1.909 mmol). The resulting red solution was purged with nitrogen and heated at 60° C. under nitrogen for 3 hours. The reaction was cooled to room temperature, diluted with MTBE (25 mL) and quenched with brine (25 mL). The aqueous layer was extracted with MTBE (3×25 mL). The combined organic extracts were dried over Na₂SO₄, filtered, and concentrated in vacuo. The crude was purified by silica gel chromatography eluting with 0-30% EtOAc in heptane to afford tert-butyl (1R,2′S)-7-chloro-2′-methyl-spiro[isochromane-1,4′-piperidine]-1′-carboxylate C88 (270 mg, 97%) ¹H NMR (400 MHz, Chloroform-d) δ 7.13 (dd, J=8.1, 2.1 Hz, 1H), 7.07-6.99 (m, 2H), 4.02 (ddd, J=11.7, 6.7, 5.4 Hz, 1H), 3.92-3.74 (m, 3H), 3.39 (ddd, J=14.3, 9.4, 5.3 Hz, 1H), 2.87-2.66 (m, 2H), 2.14 (dddd, J=15.5, 9.4, 6.1, 1.7 Hz, 1H), 2.02 (ddd, J=14.2, 5.2, 1.7 Hz, 1H), 1.93-1.74 (m, 2H), 1.52 (s, 9H), 1.28 (d, J=6.5 Hz, 3H). LCMS m/z 252.06 [M−Boc+H]⁺.

### Step 5. Synthesis of (1R,2′S)-7-chloro-2′-methyl-spiro[isochromane-1,4′-piperidine](S1)

To tert-butyl (1R,2′S)-7-chloro-2′-methyl-spiro[isochromane-1,4′-piperidine]-1′-carboxylate C88 (4 g, 10.80 mmol) was added hydrochloric acid (45 mL of 4 M, 180.0 mmol) and stirred at room temperature for 2 hours. The reaction mixture was diluted with MTBE (100 mL), and the solids filtered. The solids were washed with MTBE and vacuumed to dryness to afford a white HCl salt (1R,2′S)-7-chloro-2′-methyl-spiro[isochromane-1,4′-piperidine]S1 (3.0452 g, 97%) ¹H NMR (400 MHz, DMSO-d6) δ 9.01 (s, 2H), 7.27 (dd, J=8.1, 2.1 Hz, 1H), 7.23-7.16 (m, 2H), 3.84 (hept, J=5.8 Hz, 2H), 3.66-3.49 (m, 4H), 3.43-3.27 (m, 1H), 3.20 (dd, J=12.6, 3.8 Hz, 1H), 3.14-2.99 (m, 1H), 2.75 (t, J=5.6 Hz, 2H), 2.26 (td, J=14.0, 4.7 Hz, 1H), 2.11 (dd, J=14.3, 12.1 Hz, 1H), 1.98 (t, J=12.6 Hz, 2H), 1.27 (d, J=6.4 Hz, 3H). LCMS m/z 252.00 [M+H]⁺.

### Preparation S2

### ((1R,2′S)-7-chloro-2′-methyl-spiro[isochromane-1,4′-piperidine]-4-ol (S2)

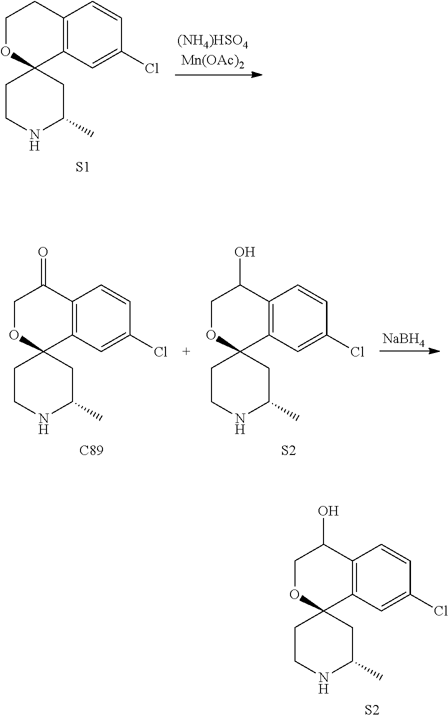

### Step 1 and 2. Synthesis of (1R,2′S)-7-chloro-2′-methyl-spiro[isochromane-1,4′-piperidine]-4-ol (S2)

To a solution of (1R,2′S)-7-chloro-2′-methyl-spiro[isochromane-1,4′-piperidine](HCl salt) S1 (50 mg, 0.1735 mmol) in acetonitrile (430 μL) and water (1.3 mL), was added ammonium hydrogen sulfate (80 mg, 0.3506 mmol) and Mn(OAc)₂ (13.7 mg, 0.07918 mmol). The reaction was stirred at 70° C. for 16 hours. Cooled to room temperature and concentrated in vacuo. The crude was carried forward the material as a mixture of the ketone C₈₉ and alcohol S2 without further purification. To the crude mixture was added methanol (2.7 mL), NaBH₄ (7.9 mg, 0.2088 mmol), and stirred at 23° C. with a needle outlet open to air for 2 hours. The reaction was concentrated and purified by reverse phase HPLC. (Method: Waters XSelect CSH C18 OBD Prep Column; 30×150 mm, 5 micron. gradient: acetonitrile in water with 0.2% formic Acid) to afford as the formic acid salt S1 (20.70 mg, 89%) LCMS m/z 268.23 [M+H]⁺.

### Preparation S3

### (1R,2′S)-7-chloro-4,4-difluoro-2′-methyl-spiro[isochromane-1,4′-piperidine](S3)

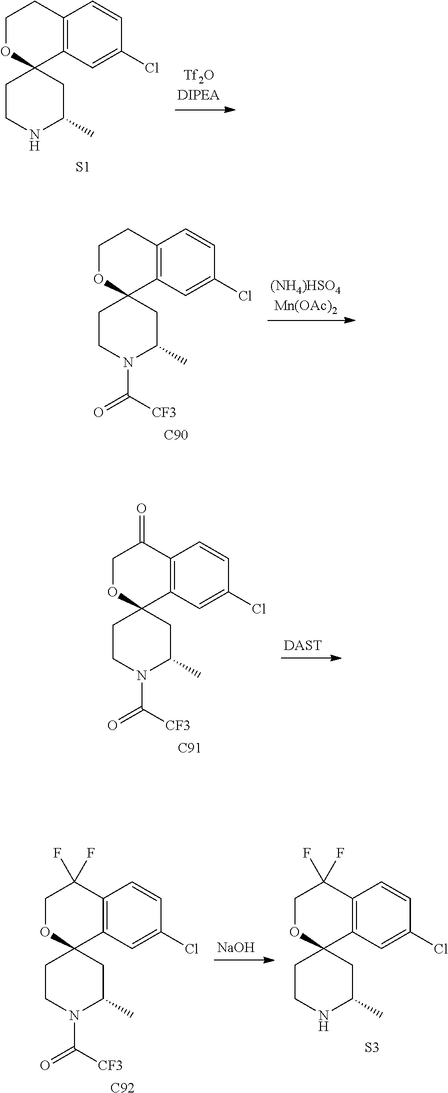

### Step 1. Synthesis of 1-[(R,2′S)-7-chloro-2′-methyl-spiro[isochromane-1,4′-piperidine]-1′-yl]-2,2,2-trifluoro-ethanone (C90)

To a solution of (1R,2′S)-7-chloro-2′-methyl-spiro[isochromane-1,4′-piperidine]1 (332 mg, 1.319 mmol) in DCM (5.6 mL), was added DIPEA (600 μL, 3.445 mmol) and trifluoromethanesulfonic anhydride (400 μL, 2.878 mmol). The reaction was stirred at 23° C. for 16 hours. The crude reaction was diluted with DCM (3 ml), and quenched with 3 ml saturated ammonium chloride. Extracted with DCM (3×10 ml) using a phase separator. The organic layer was washed with 1.5 ml of 1 M HCl, followed by 1 ml water and 1.5 ml brine. The organic was dried over sodium sulfate, filtered, and concentrated in vacuo. The crude was purified using silica gel chromatography eluting with 0-10% ethyl acetate in heptane to afford a clear glue 1-[(1R,2′S)-7-chloro-2′-methyl-spiro[isochromane-1,4′-piperidine]-1′-yl]-2,2,2-trifluoro-ethanone C₉₀ (362 mg, 77%) LCMS m/z 348.15 [M+H]⁺.

### Step 2. Synthesis of 1-[(R,2′S)-7-chloro-4-hydroxy-2′-methyl-spiro[isochromane-1,4′-piperidine]-1′-yl]-2,2,2-trifluoro-ethanone (C91)

To a solution of 1-[(1R,2′S)-7-chloro-2′-methyl-spiro[isochromane-1,4′-piperidine]-1′-yl]-2,2,2-trifluoro-ethanone C90 (1.77 g, 5.090 mmol) in water (31.5 mL) and acetonitrile (11 mL) was added ammonium hydrogen sulfate (5.8 g, 25.42 mmol) and copper (II) acetate (470 mg, 2.588 mmol). The mixture was heated at 70° C. for 16 hours. The reaction was cooled to ambient temperature, diluted with DCM (25 ml), quenched with saturated sodium bicarbonate, and separated using a phase separator (3×25 ml). The combined organic layers were dried over sodium sulfate, filtered, and concentrated in vacuo. The crude material was purified using silica gel chromatography eluting with 0-10% ethyl acetate in heptane to afford the as pale yellow solid C91 (1R,2′S)-7-chloro-2′-methyl-1′-(2,2,2-trifluoroacetyl)spiro[isochromane-1,4′-piperidine]-4-one (1.58 g, 85%) LCMS m/z 362.05 [M+H]⁺.

### Step 3. Synthesis of 1-[(1R,2′S)-7-chloro-4,4-difluoro-2′-methyl-spiro[isochromane-1,4′-piperidine]-1′-yl]-2,2,2-trifluoro-ethanone (C92)

To (1R,2′S)-7-chloro-2′-methyl-1′-(2,2,2-trifluoroacetyl)spiro[isochromane-1,4′-piperidine]-4-one C₉₁ (30.2 mg, 0.08348 mmol) was added DAST (200 μL, 1.514 mmol) sequentially over 3 days with continued stirring at 40° C. for 3 days. The reaction was cooled to room temperature, slowly quenched with saturated sodium bicarbonate. The organic layer was extracted using a phase separator with DCM (3×15 ml). The combined organic layers were dried over sodium sulfate, filtered, and concentrated in vacuo to afford, without further purification, 1-[(1R,2′S)-7-chloro-4,4-difluoro-2′-methyl-spiro[isochromane-1,4′-piperidine]-1′-yl]-2,2,2-trifluoro-ethanone C92.

### Step 4. Synthesis of (1R,2′S)-7-chloro-4,4-difluoro-2′-methyl-spiro[isochromane-1,4′-piperidine](S3)

To a solution of 1-[(1R,2′S)-7-chloro-4,4-difluoro-2′-methyl-spiro[isochromane-1,4′-piperidine]-1′-yl]-2,2,2-trifluoro-ethanone C92 in methanol (1 ml) at 0° C., was added NaOH (600 μL of 2 M, 1.200 mmol) slowly. The reaction was allowed to warm to ambient temperature for 3 hours, and then stirred at 40° C. for 16 hours. The reaction was concentrated under reduced pressure and purified by reversed phase HPLC. (Method: Waters XBridge Prep C8 Column; 30×150 mm, 5 micron. gradient: acetonitrile in water with 10 mM ammonium hydroxide) to afford a white solid (1R,2′S)-7-chloro-4,4-difluoro-2′-methyl-spiro[isochromane-1,4′-piperidine]S3 (10.2 mg, 41%) ¹H NMR (300 MHz, Chloroform-d) δ 7.66 (d, J=8.4 Hz, 1H), 7.51-7.29 (m, 2H), 4.11 (t, J=10.6 Hz, 2H), 3.26-2.92 (m, 3H), 2.10-1.79 (m, 3H), 1.68-1.55 (m, 1H), 1.24-1.06 (m, 3H). LCMS m/z 288.09 [M+H]⁺.

### Compound S4

### (1R,2′S,4S)-7-chloro-2′-methyl-spiro[isochromane-1,4′-piperidine]-4-ol(S4)

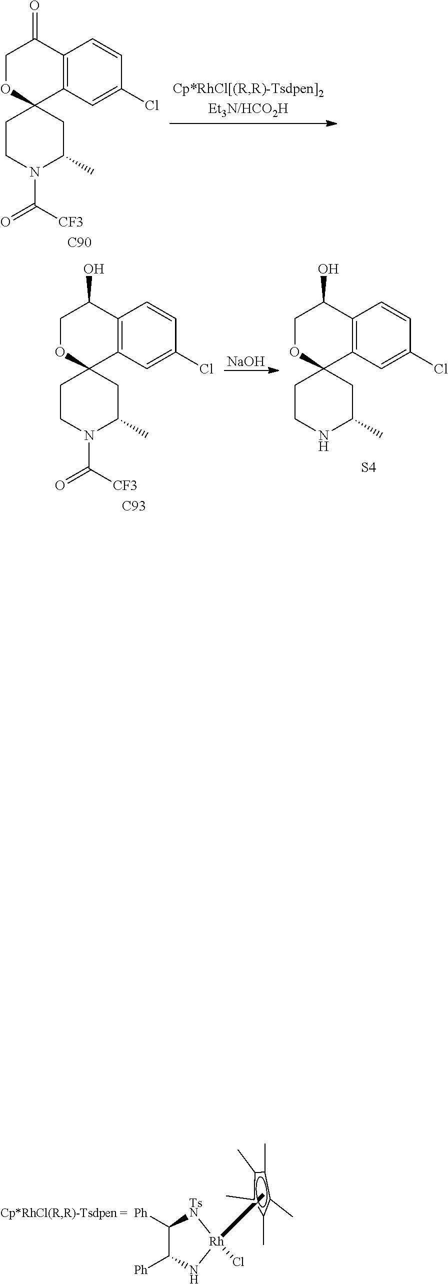

### Step 1 and 2. Synthesis of (1R,2′S,4S)-7-chloro-2′-methyl-spiro[isochromane-1,4′-piperidine]-4-ol (S4)

To a pre-mixed solution of N-[(1R,2R)-2-amino-1,2-diphenyl-ethyl]-4-methyl-benzenesulfonamide (6.9 mg, 0.01883 mmol) and dichloro(pentamethylcyclopentadienyl) rhodium(ii) dimer (3.1 mg, 0.004935 mmol) in acetonitrile (688 μl) was added a 5:2 pre-mixed solution of triethyl amine: formic acid (240 μL, 0.5716 mmol). The solution turned a bright red and some minor effervescing was observed. After 10 minutes, the mixture was cooled to 0° C. and treated with (1R,2′S)-7-chloro-2′-methyl-1′-(2,2,2-trifluoroacetyl)spiro[isochromane-1,4′-piperidine]-4-one C90 (144 mg, 0.3801 mmol) in acetonitrile (6.9 mL). The reaction was stirred at 0° C. for 1 hour, then warmed to room temperature over 1 hour. The reaction was concentrated to afford C93 without further purification. To a solution of 1-[(1R,2′S,4S)-7-chloro-4-hydroxy-2′-methyl-spiro[isochromane-1,4′-piperidine]-1′-yl]-2,2,2-trifluoro-ethanone C93, in methanol (6.9 mL) was added NaOH (950 μL of 2 M, 1.900 mmol) and stirred at 23° C. for 2 hours. The crude was concentrated and purified by reverse phase HPLC (Method: Waters XSelect CSH C18 OBD Prep Column; 30×150 mm, 5 micron. gradient: acetonitrile in water with 0.2% formic Acid) to afford as a white solid as the formic acid salt (1R,2′S,4S)-7-chloro-2′-methyl-spiro[isochromane-1,4′-piperidine]-4-ol S4 (78 mg, 65%, >99:1 dr) 1R,2′S,4S)-7-chloro-2′-methyl-spiro[isochromane-1,4′-piperidine]-4-ol (Formic Acid (1)) (78 mg, 65%) ¹H NMR (300 MHz, Chloroform-d) δ 8.51 (s, 1H), 7.47 (d, J=8.3 Hz, 1H), 7.39-7.28 (m, 1H), 7.24 (s, 1H), 4.64-4.45 (m, 1H), 3.98 (dd, J=12.1, 3.5 Hz, 1H), 3.81 (dd, J=12.1, 4.7 Hz, 1H), 3.73-3.56 (m, 1H), 3.49-3.35 (m, 2H), 2.20 (dd, J=23.3, 14.6 Hz, 2H), 2.08-1.85 (m, 2H), 1.35 (d, J=6.5 Hz, 3H). LCMS m/z 268.23 [M+H]⁺.

Note that stereochemistry of alcohol S4 was assigned based on literature understanding of reductions using this catalyst and ligand system. (Reference: New Chiral Rhodium and Iridium Complexes with Chiral Diamine Ligands for Asymmetric Transfer Hydrogenation of Aromatic Ketones. Kunihiko Murata, Takao Ikariya, and Ryoji Noyori. The Journal of Organic Chemistry 1999 64 (7), 2186-2187).

### Preparation S5

### (1R,2′S)-7-chloro-2′-methyl-1′-prop-2-ynyl-spiro[isochromane-1,4′-piperidine](S5)

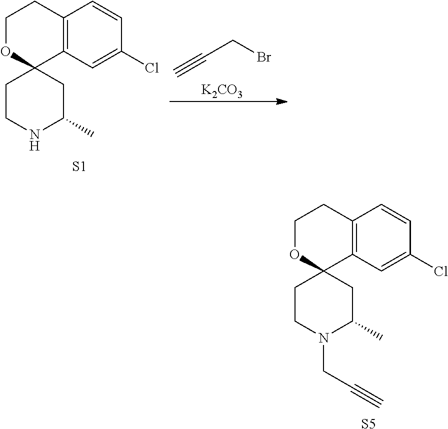

To a solution of (1R,2′S)-7-chloro-2′-methyl-spiro[isochromane-1,4′-piperidine]S1 (263 mg, 0.5223 mmol) in acetonitrile (2.6 mL) was added propargyl bromide (270 μL, 3.030 mmol, 80% v/v in toluene) and potassium carbonate (182 mg, 1.317 mmol). The reaction was stirred at 70° C. for 17 minutes. The reaction was cooled to room temperature and quenched with water (5 mL). The aqueous layer was extracted using DCM (3×10 ml). The combined organic layers were dried over sodium sulfate, filtered, and concentrated in vacuo. The crude was purified using silica gel chromatography eluting with 0-10% methanol in DCM to afford as white solid (1R,2′S)-7-chloro-2′-methyl-1′-prop-2-ynyl-spiro[isochromane-1,4′-piperidine]S5 (137.8 mg, 86%) ¹H NMR (400 MHz, Chloroform-d) δ 7.20 (d, J=2.1 Hz, 1H), 7.13 (dd, J=8.2, 2.1 Hz, 1H), 7.04 (d, J=8.2 Hz, 1H), 4.03-3.83 (m, 2H), 3.69 (dd, J=17.3, 2.4 Hz, 1H), 3.45 (dd, J=17.3, 2.5 Hz, 1H), 2.96 (td, J=11.2, 4.6 Hz, 1H), 2.89-2.64 (m, 4H), 2.27 (s, 1H), 2.08-1.96 (m, 2H), 1.96-1.87 (m, 1H), 1.72 (dd, J=13.8, 11.4 Hz, 1H), 1.12 (d, J=6.3 Hz, 3H). LCMS m/z 290.24 [M+H]⁺.

### Preparation S6

### 5-methylspiro[isochromane-1,4′-piperidine](S6)

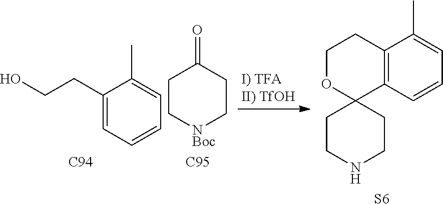

### Step 1. Synthesis of 5-methylspiro[isochromane-1,4′-piperidine]

To a solution of 2-(o-tolyl)ethanol C94 (410 mg, 2.4084 mmol) and tert-butyl 4-oxopiperidine-1-carboxylate C95 (576 mg, 2.8331 mmol) in DCM (2.5 mL), was added TFA (2.5 mL). The reaction mixture was stirred for 30 minutes at room temperature. The solvent was removed under reduced pressure. The crude material was dissolved dioxane (10 mL), cooled to 0° C., and added triflic acid (678.16 mg, 0.4 mL, 4.4284 mmol). The reaction mixture warmed to room temperature and stirred for 16 hours. The reaction mixture was quenched with 1 M NaOH (150 ml), and the aqueous layer extracted with ethyl acetate (250×2 ml). The combined organic extracts were dried over Na₂SO₄, filtered, and concentrated in vacuo to afford 5-methylspiro[isochromane-1,4′-piperidine]S6 as a yellow glue (350 mg, 16%) LCMS m/z 218.21 [M+H]⁺.

### Preparation S7

### 7-chlorospiro[isochromane-1,4′-piperidine]S7

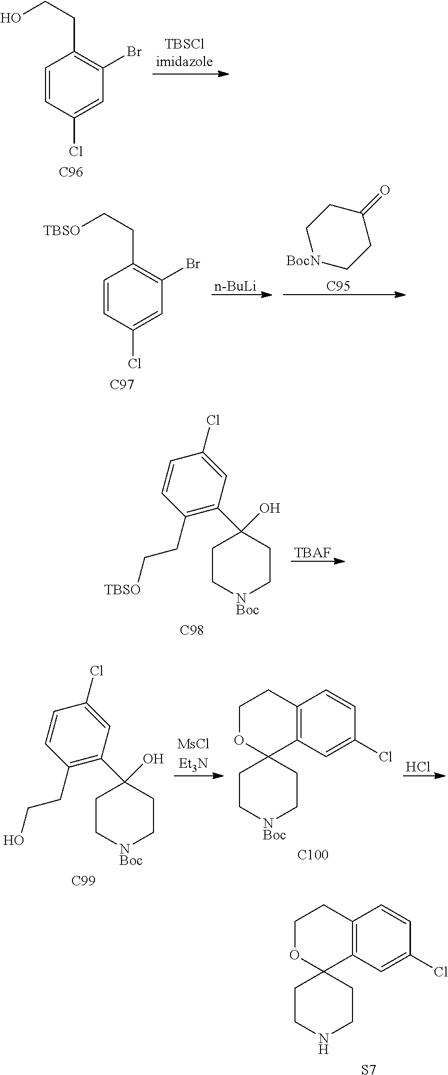

### Step 1. Synthesis of 2-(2-bromo-4-chloro-phenyl)ethoxy-tert-butyl-dimethyl-silane C97

To a solution of 2-(2-bromo-4-chloro-phenyl)ethanol C96 (1860 mg, 7.898 mmol) in DCM (16 mL) was added TBSCl (1.80 g, 11.94 mmol) and imidazole (1.08 g, 15.86 mmol). The resulting white slurry was stirred for 2.5 hours at room temperature. The reaction was diluted with DCM and quenched with 1 MN HCl. The organic layer was extracted with DCM (3×50 ml) and washed with brine (50 ml). The combined organic extracts were dried over sodium sulfate, filtered, and concentrated in vacuo. The crude material was purified using silica gel chromatography eluting with 0-50% ethyl acetate in heptane to afford a colorless oil 2-(2-bromo-4-chloro-phenyl)ethoxy-tert-butyl-dimethyl-silane C97 (2738 mg, 99%). ¹H NMR (300 MHz, Chloroform-d) δ 7.54 (d, J=1.6 Hz, 1H), 7.20 (d, J=1.8 Hz, 2H), 3.80 (t, J=6.7 Hz, 2H), 2.93 (t, J=6.7 Hz, 2H), 0.86 (s, 9H), −0.03 (s, 6H).

### Step 2. Synthesis of tert-butyl 4-[2-[2-[tert-butyl(dimethyl)silyl]oxyethyl]-5-chloro-phenyl]-4-hydroxy-piperidine-1-carboxylate C98

To a solution of 2-(2-bromo-4-chloro-phenyl)ethoxy-tert-butyl-dimethyl-silane C97 (10 g, 28.591 mmol) and tert-butyl 4-oxopiperidine-1-carboxylate (5.6967 g, 28.591 mmol) in THF (100 mL), was added n-BuLi (18.646 mL of 2.3 M, 42.886 mmol) drop wise at −78° C. and stirred for 4 h. The reaction mixture was diluted with saturated NH₄C1 solution (200 mL) and allowed to warm to room temperature. The reaction mixture was extracted with ethyl acetate (3×250 ml). The organic layer was washed with water (200 mL), dried over sodium sulfate, filtered and concentrated under reduced pressure. The crude was purified by silica gel chromatography using 0-40% ethyl acetate in heptane to afford tert-butyl 4-[2-[2-[tert-butyl(dimethyl)silyl]oxyethyl]-5-chloro-phenyl]-4-hydroxy-piperidine-1-carboxylate C98 (4 g, 30%) LCMS m/z 470.00 [M+H]⁺.

### Step 3. Synthesis of tert-butyl 4-[5-chloro-2-(2-hydroxyethyl)phenyl]-4-hydroxy-piperidine-1-carboxylate C99

To a solution of tert-butyl 4-[2-[2-[tert-butyl(dimethyl)silyl]oxyethyl]-5-chloro-phenyl]-4-hydroxy-piperidine-1-carboxylate C98 (4 g, 8.5085 mmol) in THF (10 mL), was added TBAF (12.763 mL of 1 M, 12.763 mmol) at room temperature and the reaction allowed to stir for 2 hours. The reaction mixture was extracted with ethyl acetate (2×100 ml). The combined organic layer was washed with water (80 mL), dried over sodium sulfate and concentrated under reduced pressure. The crude material was purified by silica gel chromatography eluting with 0-17% ethyl acetate in heptane to afford tert-butyl 4-[5-chloro-2-(2-hydroxyethyl)phenyl]-4-hydroxy-piperidine-1-carboxylate C9 (2.9 g, 96%) ¹H NMR (400 MHz, DMSO-d6) δ 7.39 (d, J=2.0 Hz, 1H), 7.28% E2% 80% 93 7.18 (m, 2H), 5.19 (s, 1H), 4.76 (t, J=5.1 Hz, 1H), 3.85 (d, J=12.7 Hz, 2H), 3.59 (td, J=7.2, 4.9 Hz, 2H), 3.06 (t, J=7.2 Hz, 2H), 1.86 (dt, J=12.5, 6.6 Hz, 2H), 1.75 (d, J=13.1 Hz, 2H), 1.41 (s, 9H). LCMS m/z 356.20 [M+H]⁺.

### Step 4. Synthesis of tert-butyl 7-chlorospiro[isochromane-1,4′-piperidine]-1′-carboxylate C100

To a solution of tert-butyl 4-[5-chloro-2-(2-hydroxyethyl)phenyl]-4-hydroxy-piperidine-1-carboxylate C99 (1.2 g, 3.3722 mmol) in DCM (30 mL) was added triethyl amine (853.08 mg, 1.1750 mL, 8.4305 mmol) and methanesulfonyl chloride (579.44 mg, 0.3915 mL, 5.0583 mmol). Then the reaction was stirred at ambient temperature for 2 h. Reaction mixture was diluted with water (50 mL) and extracted with DCM (50 mL×2). The organic layer was washed with brine (50 mL), dried over sodium sulfate and concentrated under reduced pressure to afford the crude material tert-butyl 7-chlorospiro[isochromane-1,4′-piperidine]-1′-carboxylate C100 (650 mg, 57%) LCMS m/z 338.00 [M+H]⁺.

### Step 5. Synthesis of 7-chlorospiro[isochromane-1,4′-piperidine]S7

To a solution of tert-butyl 7-chlorospiro[isochromane-1,4′-piperidine]-1′-carboxylate C100 (1 g, 2.960 mmol) in dioxane (23 ml) was added HCl (7.4 mL of 4 M, 29.60 mmol). The reaction was stirred at room temperature for 16 hours. The solid was filtered and dried under vacuum to afford a white solid 7-chlorospiro[isochromane-1,4′-piperidine]S7 (700 mg, 70%) LCMS m/z 238.44 [M+H]⁺.

### Preparation S8

### chlorospiro[isochromane-1,4′-piperidine](S6)

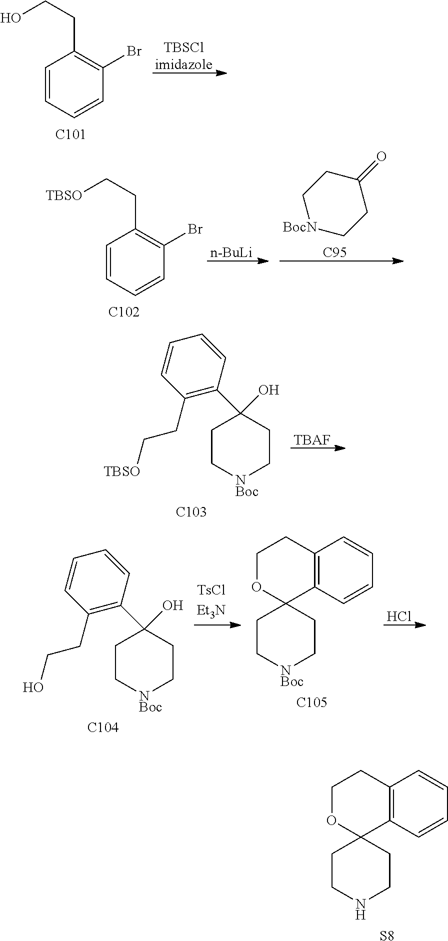

### Synthesis of spiro[isochromane-1,4′-piperidine]S8

The compound was prepared from commercially available C101 following the method described for compound S7. The reaction was evaporated to afford as a white HCl salt spiro[isochromane-1,4′-piperidine]S8 (985 mg, 89%) ¹H NMR (400 MHz, DMSO) δ 9.02 (s, 1H), 7.25 (t, J=7.3 Hz, 1H), 7.19 (t, J=6.8 Hz, 1H), 7.17-7.12 (m, 2H), 3.86 (t, J=5.5 Hz, 2H), 3.19 (dd, J=12.2, 3.3 Hz, 2H), 3.06 (td, J=12.8, 2.2 Hz, 2H), 2.77 (t, J=5.4 Hz, 2H), 2.27 (td, J=14.0, 4.6 Hz, 2H), 1.93 (d, J=14.2 Hz, 2H). LCMS m/z 204.30 [M+H]⁺.

### Preparation S9

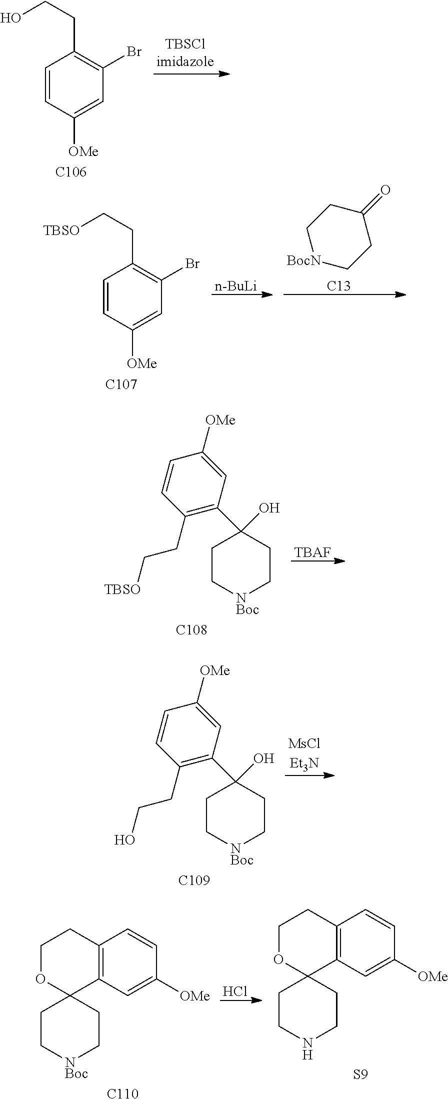

### Synthesis of 6-methoxyspiro[isochromane-1,4′-piperidine](S9)

The compound was prepared from commercially available C106 following the method described for compound S7. The reaction mixture was concentrated under reduced pressure. Saturated Na₂CO₃ solution was added to maintain the pH 10, diluted with cold (50 mL) and extracted with DCM (50 mL×2). The organic layer was dried over sodium sulfate, filtered and concentrated under reduced pressure to afford 6-methoxyspiro[isochromane-1,4′-piperidine]S9 (120 mg, 65%) LCMS m/z 234.17 [M+H]⁺.

### Preparation S10

### 7-(trifluoromethyl)spiro[isochromane-1,4′-piperidine](S10)

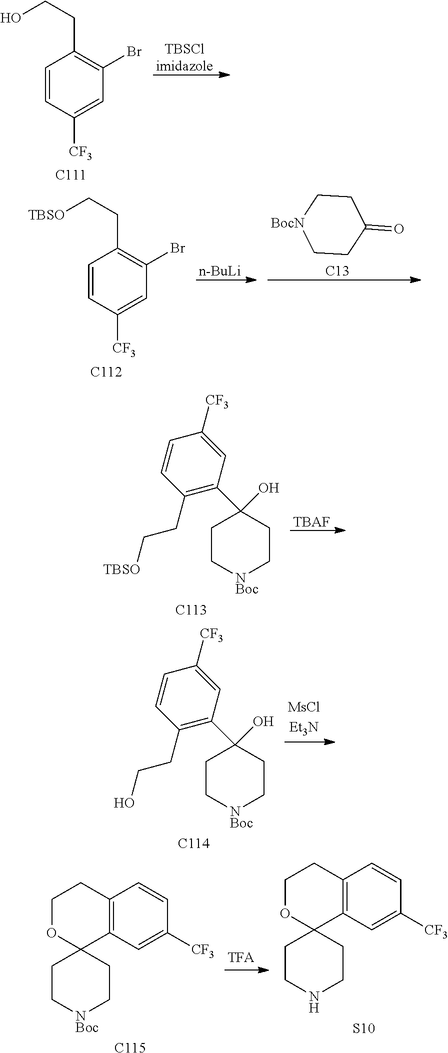

### Synthesis of 7-(trifluoromethyl)spiro[isochromane-1,4′-piperidine]S10

Compound S8 was prepared from compound C29 following the method described for compound S7. The reaction mixture was concentrated under reduced pressure to afford as the trifluoroacetate salt 7-(trifluoromethyl)spiro[isochromane-1,4′-piperidine]S10 (110 mg, 41%). LCMS m/z 272.00 [M+H]⁺.

### Preparation S11

### 7-chlorospiro[isochromane-1,4′-piperidine]-4-ol (S11)

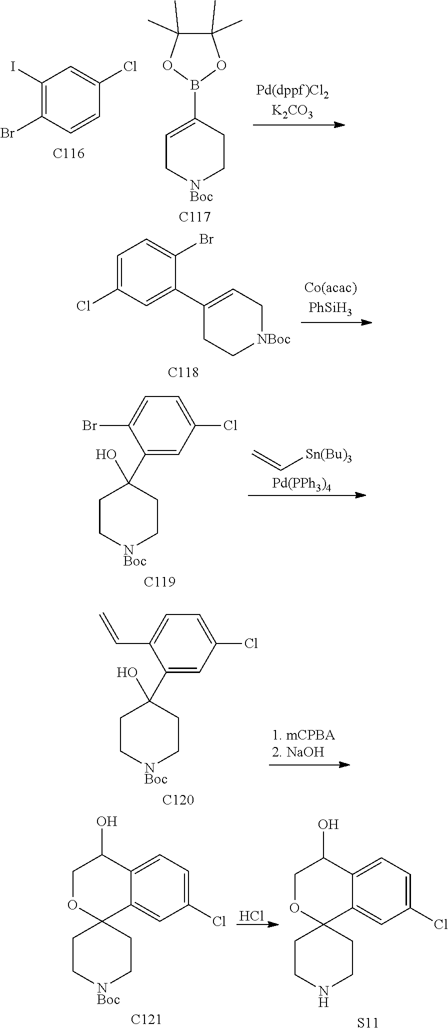

### Step 1. Synthesis of tert-butyl 4-(2-bromo-5-chloro-phenyl)-3,6-dihydro-2H-pyridine-1-carboxylate (C118)

To a solution of 1-bromo-4-chloro-2-iodo-benzene C116 (6.8 g, 21.43 mmol) in water (10 mL), and 1,4-dioxane (30 mL) was added Pd(dppf)Cl₂ (1.2 g, 1.640 mmol), K₂CO₃ (6.7 g, 48.48 mmol) and tert-butyl 4-(4,4,5,5-tetramethyl-1,3,2-dioxaborolan-2-yl)-3,6-dihydro-2H-pyridine-1-carboxylate C117 (5000 mg, 16.17 mmol). Nitrogen gas was bubbled through the stirring reaction mixture for 15 minutes. After, the reaction mixture was stirred at 80° C. for 16 hours. The reaction was warmed to room temperature, quenched with water (25 ml), extracted with ethyl acetate (3×25 ml) and washed with brine (75 ml). The combined organic layers were dried over sodium sulfate, filtered, and concentrated in vacuo. The crude material was purified by silica gel chromatography eluting with 0-15% ethyl acetate in heptane to afford the product as a yellow oil tert-butyl 4-(2-bromo-5-chloro-phenyl)-3,6-dihydro-2H-pyridine-1-carboxylate C118 (4.42 g, 71%) ¹H NMR (300 MHz, Chloroform-d) δ 7.49 (d, J=8.5 Hz, 1H), 7.18 (d, J=2.6 Hz, 1H), 7.12 (dd, J=8.4, 2.6 Hz, 1H), 5.66 (s, 1H), 4.06 (d, J=3.6 Hz, 2H), 3.64 (t, J=5.5 Hz, 2H), 2.42 (s, 2H), 1.52 (s, 9H). LCMS m/z 372.05 [M+H]⁺.

### Step 2. Synthesis of tert-butyl 4-(2-bromo-5-chloro-phenyl)-4-hydroxy-piperidine-1-carboxylate (C119)

To a solution of tert-butyl 4-(2-bromo-5-chloro-phenyl)-3,6-dihydro-2H-pyridine-1-carboxylate C118 (500 mg, 1.342 mmol) in THF (10 mL) under oxygen was added Co(acac)₂ (38 mg, 0.2147 mmol) followed by PhSiH₃ (250 μL, 2.028 mmol) and stirred for 1 day. Quenched with 1 MN sodium thiosulfate solution, extracted using ethyl acetate (3×15 ml). The combined organic layers were dried over sodium sulfate, filtered, and concentrated in vacuo. The crude material was purified using silica gel chromatography 0-35% ethyl acetate in heptane to afford tert-butyl 4-(2-bromo-5-chloro-phenyl)-4-hydroxy-piperidine-1-carboxylate C119 (280 mg, 53%) ¹H NMR (300 MHz, DMSO-d6) δ 7.84 (d, J=2.7 Hz, 1H), 7.62 (d, J=8.4 Hz, 1H), 7.28 (dd, J=8.4, 2.7 Hz, 1H), 5.52 (s, 1H), 3.86 (d, J=12.8 Hz, 2H), 3.11 (s, 2H), 2.74-2.54 (m, 4H), 1.42 (s, 9H).

### Step 3. Synthesis of tert-butyl 4-(5-chloro-2-vinyl-phenyl)-4-hydroxy-piperidine-1-carboxylate (C120)

To a solution of tert-butyl 4-(2-bromo-5-chloro-phenyl)-4-hydroxy-piperidine-1-carboxylate C119 (2000 mg, 5.119 mmol) in toluene (30 mL) under nitrogen was added tributyl(vinyl)stannane (2.5 mL, 8.554 mmol) and Pd(PPh₃)₄(180 mg, 0.1558 mmol). Sparged with nitrogen for 15 minutes and capped. The reaction was stirred at 120° C. for 1 hour. Warmed to room temperature, washed with 1 MN NaOH. The organic layer was dried over sodium sulfate, filtered and concentrate in vacuo. The crude was purified using silica gel chromatography eluting with 0-25% ethyl acetate in heptane to afford tert-butyl 4-(5-chloro-2-vinyl-phenyl)-4-hydroxy-piperidine-1-carboxylate C120 (1.2403 g, 72%). ¹H NMR (400 MHz, Chloroform-d) δ 7.58 (dd, J=17.4, 10.9 Hz, 1H), 7.45-7.35 (m, 2H), 7.29-7.24 (m, 1H), 5.52 (dd, J=17.3, 1.4 Hz, 1H), 5.35-5.27 (m, 1H), 4.09 (d, J=35.8 Hz, 2H), 3.28 (s, 2H), 2.09-1.91 (m, 4H), 1.50 (s, 9H).

### Step 4. Synthesis of tert-butyl 7-chloro-4-hydroxy-spiro[isochromane-1,4′-piperidine]-1′-carboxylate (C121)

To a solution of tert-butyl 4-(5-chloro-2-vinyl-phenyl)-4-hydroxy-piperidine-1-carboxylate (315 mg, 0.9324 mmol) in DCM (5 mL) was added mCPBA (270 mg, 1.173 mmol) was. The reaction was stirred for 16 hours. Quenched with 1 M NaOH, extracted using DCM (2×10 ml) on a PTFE frit and injected directly on a silica gel column. Eluted with 0-60% ethyl acetate in heptane to afford tert-butyl 7-chloro-4-hydroxy-spiro[isochromane-1,4′-piperidine]-1′-carboxylate C121 (200 mg, 61%) ¹H NMR (400 MHz, Chloroform-d) δ 7.25-7.18 (m, 1H), 7.07 (dt, J=8.0, 0.7 Hz, 1H), 7.00 (d, J=1.8 Hz, 1H), 5.19 (ddd, J=5.5, 3.5, 1.0 Hz, 1H), 4.05 (q, J=7.1 Hz, 2H), 3.85 (dd, J=11.7, 3.5 Hz, 1H), 3.69 (dd, J=11.7, 5.4 Hz, 1H), 3.11 (s, 2H), 1.86 (td, J=13.2, 5.0 Hz, 1H), 1.65 (q, J=11.1, 9.2 Hz, 3H), 1.42 (s, 9H). LCMS m/z 353.73 [M+H]⁺.

### Step 5. Synthesis of 7-chlorospiro[isochromane-1,4′-piperidine]-4-ol S11

To a solution of tert-butyl 7-chloro-4-hydroxy-spiro[isochromane-1,4′-piperidine]-1′-carboxylate (25 mg, 0.07065 mmol) in dioxane (1 mL) was added HCl (250 μL of 4 M, 1.000 mmol) and stirred for 3 hours. The crude reaction was purified by reverse-phase HPLC (Method: C18 Waters Sunfire column (30×150 mm, 5 micron), gradient: MeCN in H₂O with 0.1% trifluoroacetic acid) to afford 7-chlorospiro[isochromane-1,4′-piperidine]-4-ol as the trifluoroacetate salt S11 (17 mg, 62%) ¹H NMR (400 MHz, DMSO-d6) δ 9.01 (s, 2H), 7.44-7.31 (m, 2H), 7.21 (d, J=1.8 Hz, 1H), 5.16 (t, J=4.5 Hz, 1H), 4.92 (s, 1H), 3.71-3.59 (m, 2H), 3.39-3.25 (m, 2H), 3.11 (dq, J=23.0, 11.7 Hz, 2H), 2.18 (dtd, J=27.6, 13.6, 4.6 Hz, 2H), 1.90-1.72 (m, 2H). LCMS m/z 254.08 [M+H]⁺.

### Compound S12

### (2′S)-7-chloro-2′-methyl-spiro[isochromane-1,4′-piperidine](S12)

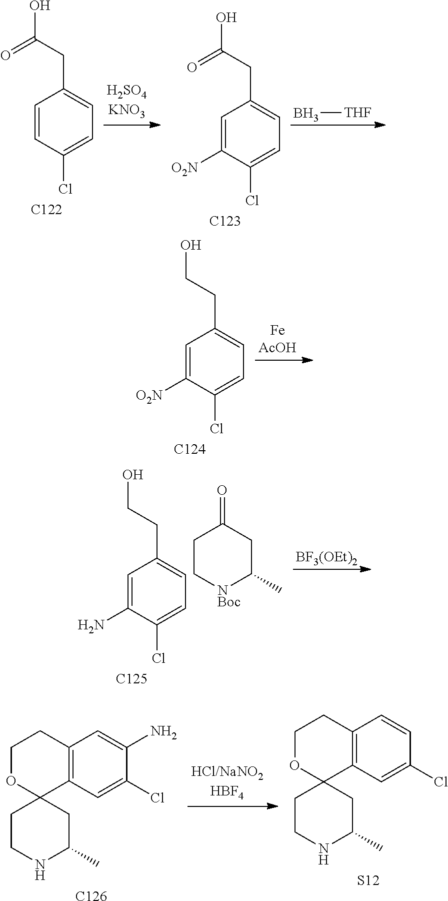

### Step 1. Synthesis of 2-(4-chloro-3-nitro-phenyl)acetic acid C123

To a solution of 2-(4-chlorophenyl)acetic acid C122 (10 g, 0.0586 mol) in H₂SO₄ (50 mL) was added potassium nitrate (6.6 g, 0.0646 mol) portion-wise at 0° C. and stirred for 2 h. The progress of the reaction was monitored by TLC with 50% ethyl acetate in hexane (0.4 rf). After completion, the reaction mass was poured into ice cold water (500 mL), the solid was filtered, washed with water and dried under reduced pressure to afford an off white solid 2-(4-chloro-3-nitro-phenyl)acetic acid C123 (10 g, 71%) ¹H NMR (400 MHz, CDCl₃) δ 7.82 (s, 1H), 7.52 (d, J=8.0 Hz, 1H), 7.46-7.43 (m, 1H), 3.72 (s, 2H).

### Step 2. Synthesis of 2-(4-chloro-3-nitro-phenyl)ethanol C124

To a solution of 2-(4-chloro-3-nitro-phenyl)acetic acid C123 (8 g, 0.0371 mol) in THF (80.000 mL) was added BH₃ (solution in THF) (55.700 mL of 1 M, 0.0557 mol) slowly at 0° C. The reaction was heated at 60° C. for 16 h. Cooled the reaction to ambient temperature, quenched with 1 M HCl (50 ml), added water (250 ml) and extracted with ethyl acetate (2×300 ml). The combined organic layers were dried over sodium sulfate, filtered, and concentrated under reduced pressure. The crude was purified using silica gel chromatography using 0-20% ethyl acetate in petroleum ether to afford a yellow oil 2-(4-chloro-3-nitro-phenyl)ethanol C124 (7 g, 92%) (400 MHz, CDCl₃), δ ppm 7.77 (d, J=1.6 Hz, 1H), 7.48-7.46 (m, 1H), 7.42-7.39 (m, 1H), 3.905 (t, J=6.4 Hz, 2H), 2.91 (t, J=6.4 Hz, 2H).

### Step 3. Synthesis of 2-(3-amino-4-chloro-phenyl)ethanol C125

To a solution of 2-(4-chloro-3-nitro-phenyl)ethanol C124 (420 mg, 0.0015 mol) in acetic acid (5.0 mL) was added Fe (503 mg, 0.0090 mol) at room temperature and the reaction mixture was stirred for 80° C. for 1 h. The progress of the reaction was monitored by TLC with 50% ethyl acetate in hexane (0.3 rf). After completion, the reaction mass was filtered, washed with ethyl acetate (100 mL). The filtrate was washed with sodium bicarbonate solution (100 mL), the organic layer was dried over sodium sulfate, and concentrated under reduced pressure. The crude compound was purified by silica gel chromatography eluting with 40% ethyl acetate in hexane to afford 2-(3-amino-4-chloro-phenyl)ethanol C125 (200 mg, 76%) ¹H NMR (400 MHz, CDCl₃) δ 7.17 (d, J=8.0 Hz, 1H), 6.44 (s, 1H), 6.55 (dd, J=8.4, 2.0 Hz, 1H), 3.821 (t, J=6.4 Hz, 2H), 2.751 (t, J=6.4 Hz, 2H). LCMS m/z 172.13 [M+H]⁺.

### Step 4. Synthesis of (2′S)-7-chloro-2′-methyl-spiro[isochromane-1,4′-piperidine]-6-amine C126

To a solution of 2-(3-amino-4-chloro-phenyl)ethanol C125 (100 mg, 582.67 μmol) in toluene (1.5 mL) was added tert-butyl (2S)-2-methyl-4-oxo-piperidine-1-carboxylate C1 (149.12 mg, 699.20 μmol) and then added BF₃·OEt₂ (411.59 mg, 0.3642 mL, 0.0029 mol) at ambient temperature. The reaction was stirred at 120° C. for 16 h. The reaction was concentrated under reduced pressure and basified with saturated sodium bicarbonate solution (10 ml), extracted with ethyl acetate (2×50 ml). The combined organic layers were dried over sodium sulfate, filtered and concentrated under reduced pressure to afford (2′S)-7-chloro-2′-methyl-spiro[isochromane-1,4′-piperidine]-6-amine C126 (150 mg, 49%) LCMS m/z 267.00 [M+H]⁺.

### Step 6. Synthesis of (2′S)-7-chloro-2′-methyl-spiro[isochromane-1,4′-piperidine]S12

To a solution of (2′S)-7-chloro-2′-methyl-spiro[isochromane-1,4′-piperidine]-6-amine C126 (50 mg, 129.33 μmol) in HCl (3.0000 mL of 2 M, 0.0060 mol) was added NaNO₂ (11.896 mg, 129.33 μmol) at ambient temperature, then cooled to 0° C. Fluoroboric acid (0.0341 mL of 50% w/v, 194.00 μmol) was added slowly at 0° C. and the reaction was warmed to ambient temperature and stirred for 16 hours. The reaction was quenched with water (10 ml), basified with saturated sodium bicarbonate solution (5 ml), and extracted with ethyl acetate (2×50 ml). The combined organic layers were dried over sodium sulfate, filtered and concentrated under reduced pressure to afford (2′S)-7-chloro-2′-methyl-spiro[isochromane-1,4′-piperidine]3 (25 mg, 28%) LCMS m/z 252.00 [M+H]⁺.

### Preparation S13

### 7-chloro-3′-methylspiro[isochromane-1,4′-piperidine] (S13)

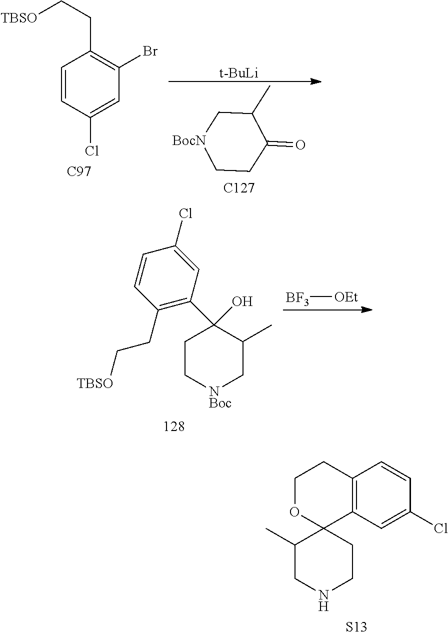

### Step 2. Synthesis of tert-butyl 4-[2-[2-[tert-butyl(dimethyl)silyl]oxyethyl]-5-chloro-phenyl]-4-hydroxy-3-methyl-piperidine-1-carboxylate C128

To a solution of 2-(2-bromo-4-chloro-phenyl)ethoxy-tert-butyl-dimethyl-silane C97 (1 g, 0.0029 mol) in THF (15.000 mL) was added t-BuLi (2.0588 mL of 1.7 M, 0.0035 mol) slowly at −100° C. over 10 minutes. The reaction was stirred for 30 minutes at −100° C., then tert-butyl 3-methyl-4-oxo-piperidine-1-carboxylate C127 (746.46 mg, 0.0035 mol) (in 1 ml THF) was added slowly over 15 min at −100° C. The reaction was stirred for 30 minutes at same temperature, then slowly warmed to ambient temperature, and stirred for 1 h. The reaction was quenched with saturated ammonium chloride solution (20 ml) and extracted with ethyl acetate (2×100 ml). The combined organic layers were dried over sodium sulfate, filtered abd concentrated under reduced pressure to afford tert-butyl 4-[2-[2-[tert-butyl(dimethyl)silyl]oxyethyl]-5-chloro-phenyl]-4-hydroxy-3-methyl-piperidine-1-carboxylate C128 (1.5 g, 31%) LCMS m/z 484.00 [M+H]⁺.

### Step 3. Synthesis of 7-chloro-3′-methyl-spiro[isochromane-1,4′-piperidine]S13

To a solution of tert-butyl 4-[2-[2-[tert-butyl(dimethyl)silyl]oxyethyl]-5-chloro-phenyl]-4-hydroxy-3-methyl-piperidine-1-carboxylate C128 (100 mg, 206.55 μmol) in toluene (1.5 mL) was added BF₃·OEt₂ (298.05 mg, 0.2638 mL, 0.0021 mol) at room temperature, then the reaction was stirred for 16 h at ambient temperature while monitoring by TLC until completion. The reaction was concentrated under reduced pressure, basified with saturated sodium bicarbonate solution (20 ml) and extracted with ethyl acetate (2×100 ml). The combined organic layer were dried over sodium sulfate, filtered and concentrated under reduced pressure to afford 7-chloro-3′-methyl-spiro[isochromane-1,4′-piperidine]S13 (40 mg, 42%) LCMS m/z 252.00 [M+H]⁺.

### Compound S14

### (2′S)-2′-methylspiro[isochromane-1,4′-piperidine](S14)

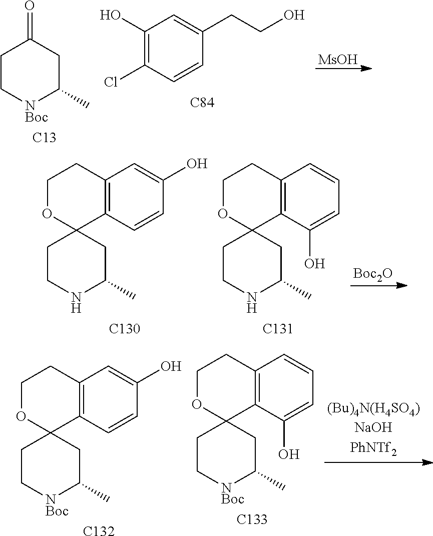

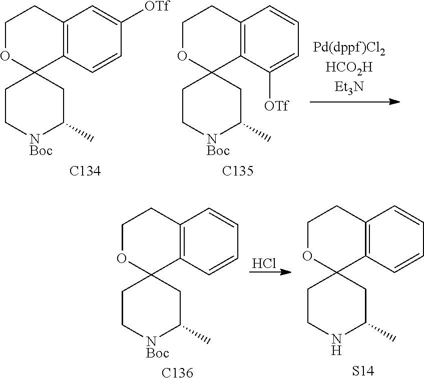

Compound S14 was prepared from compound C13 following the method described for compound S1. The crude reaction was purified by reverse phase HPLC (Method: Waters XSelect CSH C18 OBD Prep Column; 30×150 mm, 5 micron. gradient: acetonitrile in water with 5 mM hydrochloric acid) to afford as the HCl salt 2′S)-2′-methylspiro[isochromane-1,4′-piperidine]S14 (26 mg, 47%) ¹H NMR (300 MHz, Chloroform-d) δ 7.33-7.08 (m, 3H), 3.93 (t, J=5.6 Hz, 2H), 3.62 (s, 1H), 3.42 (dd, J=12.6, 3.8 Hz, 1H), 2.83 (t, J=5.6 Hz, 2H), 2.31-1.87 (m, 4H), 1.36 (d, J=6.6 Hz, 3H). LCMS m/z 218.24 [M+H]⁺.

### Preparation C136 and C137

### tert-butyl (1R,2′S)-2′-methyl-6-(trifluoromethylsulfonyloxy)spiro[isochromane-1,4′-piperidine]-1′-carboxylate (C136) and tert-butyl (1S,2′S)-2′-methyl-6-(trifluoromethylsulfonyloxy)spiro[isochromane-1,4′-piperidine]-1′-carboxylate (C137)

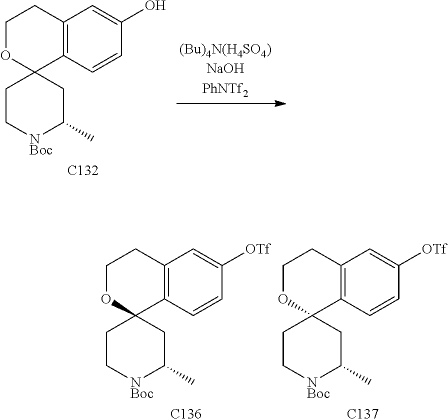

### Synthesis of tert-butyl (1R,2′S)-2′-methyl-6-(trifluoromethylsulfonyloxy)spiro[isochromane-1,4′-piperidine]-1′-carboxylate (C136) and tert-butyl (1S,2′S)-2′-methyl-6-(trifluoromethylsulfonyloxy)spiro[isochromane-1,4′-piperidine]-1′-carboxylate (C137)

To a solution of tert-butyl (2′S)-6-hydroxy-2′-methyl-spiro[isochromane-1,4′-piperidine]-1′-carboxylate C132 (766.1 mg, 2.298 mmol) in DCM (18 mL), was added 1,1,1-trifluoro-N-phenyl-N-(trifluoromethylsulfonyl)methanesulfonamide (2.1 g, 5.878 mmol), tetrabutylammonium hydrogen sulfate (1.1 g, 3.240 mmol), followed by NaOH (8.5 mL of 2 M, 17.00 mmol). Stirred at room temperature for 16 hours. The organic layer was separated using a phase separators and DCM (3×25 ml). The combined organic layers were dried over sodium sulfate, filtered, and concentrated in vacuo. The crude was purified using silica gel chromatography using 0-10% ethyl acetate in hexanes to afford two diastereomers. The absolute stereochemistry was confirmed by 1HNMR analysis. Tert-butyl (1R, 2′S)-2′-methyl-6-(trifluoromethylsulfonyloxy)spiro[isochromane-1,4′-piperidine]-1′-carboxylate C136 (425.6 mg, 79%) LCMS m/z 366.01 [M-Boc+H]⁺ and tert-butyl (1S,2′S)-2′-methyl-6-(trifluoromethylsulfonyloxy)spiro[isochromane-1,4′-piperidine]-1′-carboxylate C137 (240.8 mg, 42%) LCMS m/z 366.01 [M-Boc+H]⁺

### Compound S15

### (1R,2′S)-6-chloro-2′-methyl-spiro[isochromane-1,4′-piperidine](S15)

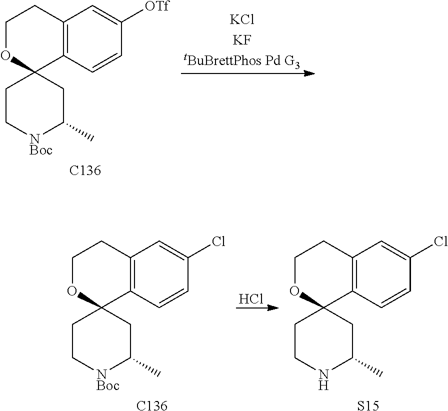

### Step 1 and 2. Synthesis of (1R, 2′S)-6-chloro-2′-methyl-spiro[isochromane-1,4′-piperidine](S15)

To tert-butyl (2′S)-2′-methyl-6-(trifluoromethylsulfonyloxy)spiro[isochromane-1,4′-piperidine]-1′-carboxylate (307 mg, 0.6595 mmol) was added tBuBrettPhos Pd G3 (59.34 mg, 0.06945 mmol), KCl (127 mg, 1.704 mmol), KF (25.42 mg, 0.4375 mmol) and purged with nitrogen 3 times. Added dioxane (3.4 mL) and heated to 130° C. for 24 h. Cooled the reaction to 23° C., added HCl (4.397 mL of 6 M, 26.38 mmol) and stirred for 16 hours. Concentrated the crude reaction material. The crude reaction was purified using reverse phase HPLC (Method: Waters XBridge Prep C8 Column; 30×150 mm, 5 micron. gradient: acetonitrile in water with 10 mM ammonium hydroxide to afford (1R, 2′S)-6-chloro-2′-methyl-spiro[isochromane-1,4′-piperidine]S15 (27 mg, 16%) ¹H NMR (300 MHz, Chloroform-d) δ 7.28-6.96 (m, 3H), 3.94-3.81 (m, 2H), 3.48-3.35 (m, 2H), 2.96-2.64 (m, 3H), 2.11-1.75 (m, 4H), 1.43 (d, J=7.0 Hz, 3H). LCMS m/z 252.20 [M+H]⁺.

### Preparation C140

### (2′S)-2′, 7-dimethylspiro[isochromane-1,4′-piperidine](C140)

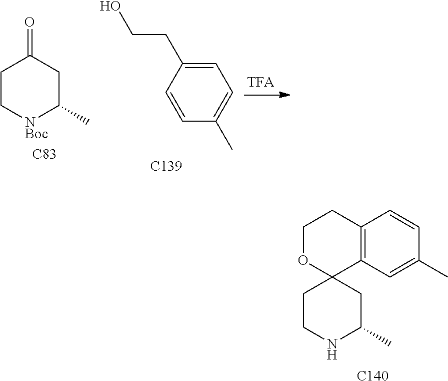

The compound C140 was prepared from C83 and C139 following the method described for compound S6. The reaction was basified with 1 M NaOH (20 ml), extracted with EtOAc (2×100 ml). The combined organic layers were dried over sodium sulfate, filtered, and concentrated under reduced pressure to afford the product (2′S)-2′,7-dimethylspiro[isochromane-1,4′-piperidine]C140 (180 mg, 36%) LCMS m/z 232.00 [M+H]⁺.

### Preparation Compound S16

### 2′,7-dimethylspiro[isochromane-1,4′-piperidine](S16)

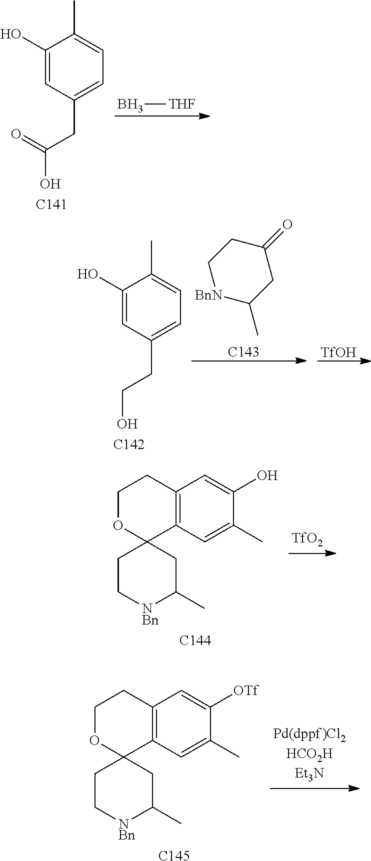

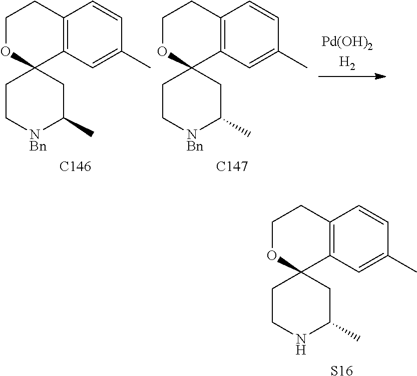

### Step 1. Synthesis of 5-(2-hydroxyethyl)-2-methyl-phenol (C142)

To a solution of 2-(3-hydroxy-4-methyl-phenyl)acetic acid C141 (890 mg, 5.356 mmol) in THF (11 mL) at 0° C. was added slowly BH₃-THF (10.7 mL of 1 M, 10.70 mmol). The reaction was warmed to room temperature and stirred overnight for 16 hours. The reaction was cooled down to 0° C. and quenched slowly quenched with methanol (12 ml) until effervescence subsided. The reaction was concentrated in vacuo. The crude material was purified using silica gel chromatography eluting with 0-100% ethyl acetate in heptane to afford 5-(2-hydroxyethyl)-2-methyl-phenol C142 (800 mg, 98%). ¹H NMR (300 MHz, Chloroform-d) δ 7.06 (d, J=7.5 Hz, 1H), 6.71 (dd, J=7.6, 1.7 Hz, 1H), 6.66 (d, J=1.7 Hz, 1H), 4.83 (s, 1H), 3.84 (s, 2H), 2.79 (t, J=6.5 Hz, 2H), 2.22 (s, 3H), 1.43 (s, 1H). LCMS m/z 135.00 [M−OH+H]⁺.

### Step 2. Synthesis of 1′-benzyl-2′, 7-dimethyl-spiro[isochromane-1,4′-piperidine]-6-ol (C144)

To a solution of 1-benzyl-2-methyl-piperidin-4-one C142 (150 mg, 0.7379 mmol), and 1-benzylpiperid-4-one C143 (124 mg, 0.8148 mmol) in dioxane (2.3 mL) at 0° C. was added triflic acid (221 mg, 1.473 mmol) slowly. The reaction was warmed to room temperature and stirred for 2.5 hours. The reaction was quenched with sat. NaHCO₃, extracted with DCM (15 ml×3). The combined organic layers were dried over sodium sulfate, filtered, and concentrated in vacuo. The material was carried forward as a mixture of diastereomers. 1′-benzyl-2′,7-dimethyl-spiro[isochromane-1,4′-piperidine]-6-ol C61 (310 mg, 125%) LCMS m/z 338.00 [M+H]⁺.

### Step 3. Synthesis of (1′-benzyl-2′, 7-dimethyl-spiro[isochromane-1,4′-piperidine]-6-yl) trifluoromethanesulfonate (C145)

To a solution of 1′-benzyl-2′,7-dimethyl-spiro[isochromane-1,4′-piperidine]-6-ol C144 (249 mg, 0.7379 mmol) in DCM (3.7 mL) at 0° C. was added pyridine (134 μL, 1.2587 mmol) followed by triflic anhydride (149 μL, 0.8856 mmol). The resulting yellow solution was warmed to room temperature and stirred for 16 h. The reaction was quenched with saturated. NaHCO₃, extracted with DCM (8 ml×c3) using a phase separator. The combined organic layers were dried over sodium sulfate, filtered, and concentrated in vacuo to afford (1′-benzyl-2′,7-dimethyl-spiro[isochromane-1,4′-piperidine]-6-yl) trifluoromethanesulfonate C145 (346 mg, >99%) LCMS m/z 470.00 [M+H]⁺.

### Step 4. Synthesis of (cis) 1′-benzyl-2′,7-dimethyl-spiro[isochromane-1,4′-piperidine]C146 and (trans) 1′-benzyl-2′,7-dimethyl-spiro[isochromane-1,4′-piperidine]C147

To (1′-benzyl-2′,7-dimethyl-spiro[isochromane-1,4′-piperidine]-6-yl) trifluoromethanesulfonate C145 (346 mg, 0.7369 mmol) was added and Pd(dppf)C12 (60 mg, 0.07347 mmol). Purged thrice with nitrogen. Under nitrogen, added DMF (3.7 mL), triethyl amine (308 μL, 2.210 mmol) and formic acid (56 μL, 1.484 mmol). The resulting solution was stirred at 60° C. for 16 hours. The reaction was concentrated under vacuum. The crude was diluted with DCM (5 mL), quenched with saturated sodium bicarbonate. The organic layer was extracted using DCM (5 mL×2). The combined organic layers were dried over sodium sulfate, filtered, and concentrated in vacuo. 20 mg of the crude was purified by reverse-phase HPLC (Method: C18 Waters Sunfire column (30×150 mm, 5 micron), gradient: MeCN in H₂O with 0.1% trifluoroacetic acid) to afford as a mixture of cis and trans diastereomers C146 and C147 1′-benzyl-2′,7-dimethyl-spiro[isochromane-1,4′-piperidine](Trifluoroacetate salt) (11.8 mg, 3%) ¹H NMR (300 MHz, Chloroform-d) δ 7.52 (dd, J=6.9, 3.7 Hz, 5H), 7.01 (s, 2H), 6.92 (s, 1H), 4.85 (d, J=13.1 Hz, 1H), 4.13 (d, J=13.1 Hz, 1H), 3.96-3.81 (m, 2H), 3.74 (dt, J=11.7, 6.1 Hz, 1H), 3.25-3.35 (m, 1H), 3.18 (dt, J=12.4, 3.5 Hz, 1H), 2.77 (q, J=5.3 Hz, 2H), 2.29 (s, 3H), 2.18 (d, J=9.9 Hz, 2H), 2.09 (dd, J=11.5, 4.1 Hz, 2H), 1.61 (d, J=6.5 Hz, 3H). LCMS m/z 322.31 [M+H]⁺. The rest of the material was purified using reverse-phase HPLC (Method: C18 Waters Sunfire column (30×150 mm, 5 micron), gradient: MeCN in H₂O with 0.1% trifluoroacetic acid) to afford the cis and trans diastereomers as trifluoroacetic acid salts (cis) 1′-benzyl-2′,7-dimethyl-spiro[isochromane-1,4′-piperidine](Trifluoroacetate salt) C146 (11.8 mg, 3%) ¹H NMR (300 MHz, Chloroform-d) δ 7.52 (dd, J=6.9, 3.7 Hz, 5H), 7.01 (s, 2H), 6.92 (s, 1H), 4.85 (d, J=13.1 Hz, 1H), 4.13 (d, J=13.1 Hz, 1H), 3.96-3.81 (m, 2H), 3.74 (dt, J=11.7, 6.1 Hz, 1H), 3.25-3.35 (m, 1H), 3.18 (dt, J=12.4, 3.5 Hz, 1H), 2.77 (q, J=5.3 Hz, 2H), 2.29 (s, 3H), 2.18 (d, J=9.9 Hz, 2H), 2.09 (dd, J=11.5, 4.1 Hz, 2H), 1.61 (d, J=6.5 Hz, 3H). LCMS m/z 322.31 [M+H]⁺ and (trans) 1′-benzyl-2′,7-dimethyl-spiro[isochromane-1,4′-piperidine](Trifluoroacetate salt) C147 (4.1 mg, 1%) ¹H NMR (300 MHz, Chloroform-d) δ 7.61 (dd, J=6.6, 2.9 Hz, 2H), 7.52 (q, J=3.4, 2.8 Hz, 3H), 7.02 (d, J=10.4 Hz, 2H), 6.91 (s, 1H), 4.44-4.28 (m, 2H), 3.92 (dd, J=6.5, 4.6 Hz, 2H), 3.79 (q, J=7.0 Hz, 1H), 3.70-3.55 (m, 1H), 3.30-3.15 (m, 1H), 2.79 (dt, J=9.9, 5.5 Hz, 2H), 2.44-2.32 (m, 2H), 2.25 (d, J=13.3 Hz, 4H), 2.14-2.00 (m, 1H), 1.68 (d, J=7.2 Hz, 3H). LCMS m/z 322.00 [M+H]⁺.

### Step 5. Synthesis of 2′, 7-dimethylspiro[isochromane-1,4′-piperidine](S16)

To a solution of (trans) 1′-benzyl-2′,7-dimethyl-spiro[isochromane-1,4′-piperidine](Trifluoroacetate salt) C147 (160 mg, 0.4977 mmol) in MeOH (3.3 mL) was added Pd(OH)₂ (35 mg, 0.04985 mmol). The resulting slurry was bubbled with H₂ for 1 minute, and then stirred under hydrogen atmosphere using a balloon for 4 hours. The reaction was filtered through celite, washed with MeOH, and concentrated in vacuo to afford a white form solid. 14 mg of the crude was purified by reverse-phase HPLC (Method: C18 Waters Sunfire column (30×150 mm, 5 micron), gradient: MeCN in H₂O with 0.1% trifluoroacetic acid) to afford as the triflate salt 4:1 trans: cis 2′,7-dimethylspiro[isochromane-1,4′-piperidine]S16 (12.6 mg, 7%) LCMS m/z 232.00 [M+H]⁺.

### Preparation S17

### 4-methylspiro[isochromane-1,4′-piperidine](S17)

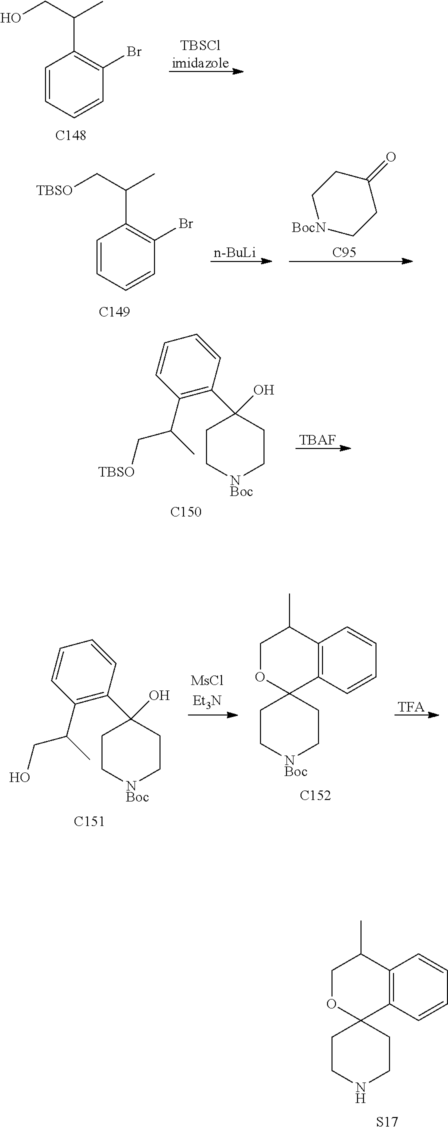

Compound S17 was prepared from compound C148 following the method described for compound S7. The crude reaction was quenched with saturated sodium bicarbonate to achieve pH 10, diluted with cold water (50 mL) and extracted with DCM (50 mL×2). The organic layer was dried over Na₂SO₄, filtered and concentrated under reduced pressure to afford the crude 4-methylspiro[isochromane-1,4′-piperidine]S17 (170 mg, 87%) LCMS m/z 218.2 (M+H)⁺.

### Preparation S18

### 1-(2-methylsulfonylethyl)triazole-4-carbaldehyde (S18)

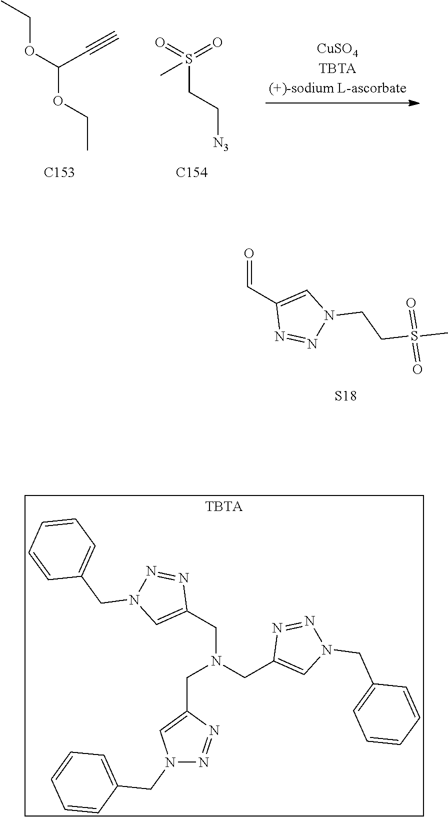

### Synthesis of 1-(2-methylsulfonylethyl)triazole-4-carbaldehyde (S18)

To a solution of 1-azido-2-methylsulfonyl-ethane C154 (2 g, 0.0134 mol) in in MeOH (80.000 mL) and H₂O (10.000 mL) was added 3,3-diethoxyprop-1-yne C153 (1.6662 g, 0.0130 mol), (+)-sodium L-ascorbate (2.6348 g, 0.0133 mol) and CuSO₄ (49.191 mg, 308.20 μmol). The reaction was stirred at 65° C. for 4 h and cooled to ambient temperature. The reaction was diluted with ethyl acetate (150 mL), water (100 mL) and stirred for 20 minutes. The organic layer was separated, and aqueous layer was extracted using ethyl acetate (2×50 ml). The combined organic layers were dried over sodium sulfate and concentrated under reduced pressure. The material was suspended in 1 M HCl (80 mL) and stirred for 24 h at ambient temperature. The reaction mixture was concentrated in vacuo to afford 1-(2-methylsulfonylethyl)triazole-4-carbaldehyde S18 (1.2 g, 33%) ¹H NMR (300 MHz, DMSO-d6) δ=8.27 (s, 1H), 5.65 (s, 1H), 5.07-4.97 (t, J=6.6 Hz, 2H), 3.07-3.03 (t, J=6.6 Hz, 2H), 2.97 (s, 3H). LCMS m/z 203.90 [M+H]⁺.

### Compound 88

### (1R,2′S)-7-chloro-2′-methyl-1′-[[1-(2-methylsulfonylethyl)pyrazol-4-yl]methyl]spiro[isochromane-1,4′-piperidine](88)

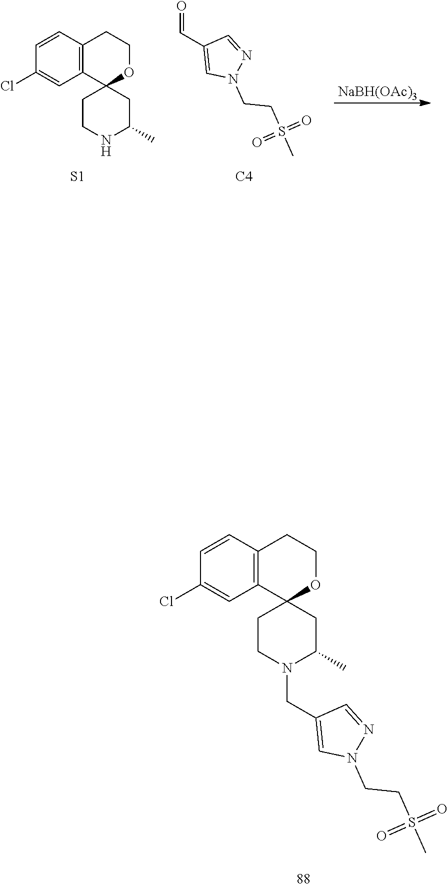 [SMILES: C[C@H]1C[C@@]2(CCN1)OCCc1ccc(Cl)cc12 ; CS(=O)(=O)CCn1cc(C=O)cn1 ; "S1" ; "C4" ; C[C@H]1C[C@@]2(CCN1Cc1cnn(CCS(C)(=O)=O)c1)OCCc1ccc(Cl)cc12 ; "NaBH(OAc)3"]

Reductive amination: Standard Method A

### Synthesis of (1R,2′S)-7-chloro-2′-methyl-1′-[[1-(2-methylsulfonylethyl)pyrazol-4-yl]methyl]spiro[isochromane-1,4′-piperidine](88)

To a solution of (1R,2′S)-7-chloro-2′-methyl-spiro[isochromane-1,4′-piperidine]1 (24.4 mg, 0.084 mmol) in 1,2-dichloroethane (1.7 ml), was added 1-(2-methylsulfonylethyl)pyrazole-4-carbaldehyde S12 (23.7 mg), NaBH(OAc)₃ (53 mg) and stirred at 80° C. for 16 hours. The reaction was warmed to room temperature, filtered through a silica gel plug and concentrated. The crude material was purified by reverse phase HPLC (Method: Waters XSelect CSH C18 OBD Prep Column; 30×150 mm, 5 micron. gradient: acetonitrile in water with 0.1% trifluoroacetic acid) to afford as the trifluoroacetic acid salt (1R,2′S)-7-chloro-2′-methyl-1′-[[1-(2-methylsulfonylethyl)pyrazol-4-yl]methyl]spiro[isochromane-1,4′-piperidine]88 (28.2 mg, 59%) ¹H NMR (300 MHz, Chloroform-d) δ 8.01 (s, 1H), 7.75 (d, J=0.8 Hz, 1H), 7.28-7.10 (m, 3H), 4.76-4.66 (m, 2H), 4.55 (d, J=14.1 Hz, 1H), 4.31 (d, J=14.1 Hz, 1H), 3.97-3.81 (m, 3H), 3.73 (dd, J=6.8, 5.5 Hz, 2H), 3.59-3.43 (m, 1H), 2.87 (d, J=0.7 Hz, 3H), 2.85-2.73 (m, 3H), 2.28-1.95 (m, 4H), 1.54 (d, J=6.4 Hz, 3H). LCMS m/z 438.31 [M+H]⁺.

### Preparation Compound 89

### 4-methyl-1′-[(1-methylpyrazol-4-yl)methyl]spiro[isochromane-1,4′-piperidine](89)

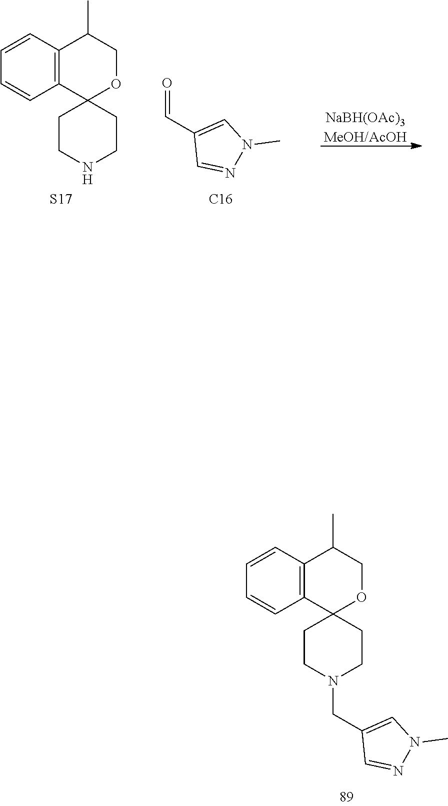

### Synthesis of 4-methyl-1′-[(1-methylpyrazol-4-yl)methyl]spiro[isochromane-1,4′-piperidine](89)

### Reductive Amination: Standard Method B

To a solution of 4-methylspiro[isochromane-1,4′-piperidine]S17 (300 mg, 1.3805 mmol) in methanol (3 mL),1-methylpyrazole-3-carbaldehyde C74 (152.01 mg, 1.3805 mmol) and catalytic amount of acetic acid were added. After 1 h, sodium cyanoborohydride (260.26 mg, 4.1415 mmol) was added and the reaction mixture was stirred at room temperature for 16 h. Reaction mixture was concentrated under reduced pressure, diluted with cold water (50 mL) and extracted with DCM (50 mL×2). The organic layer was dried over Na₂SO₄, filtered, and concentrated under reduced pressure. The crude material was purified by reverse phase HPLC (Waters auto purification instrument. Column name: YMC Triart Actus C18 (250×20 mm, 5μ) operating at ambient temperature and flow rate of 16 mL/min. Mobile phase: A=20 mM ammonium bicarbonate in water, B=acetonitrile; gradient profile: mobile phase initial composition of 80% A and 20% B, then 70% A and 30% B in 3 min, then to 45% A and 55% B in 20 min., then to 5% A and 95% B in 21 min., held this composition up to 23 min. for column washing, then returned to initial composition in 24 min. and held until 26 min) to afford 4-methyl-1′-[(1-methylpyrazol-4-yl)methyl]spiro[isochromane-1,4′-piperidine]89 (150 mg, 34%) H NMR (400 MHz, DMSO-d6) δ 7.56 (s, 1H), 7.30 (s, 1H), 7.16 (s, 4H), 3.82-3.80 (m, 1H), 3.82 (s, 3H), 3.51 (dd, J=11.4, 4.3 Hz, 1H), 3.36 (s, 2H), 2.77-2.76 (m, 1H), 2.63-2.60 (m, 2H), 2.29-2.19 (m, 2H), 1.97-1.94 (m, 1H), 1.81-1.79 (m, 2H), 1.71-1.67 (m, 1H), 1.20 (d, J=7.0 Hz, 3H). LCMS m/z 312.00 [M+H]⁺.

### Preparation Compound 90

### (1R,2′S)-7-chloro-2′-methyl-1′-[[1-(2-methylsulfonylethyl)triazol-4-yl]methyl]spiro[isochromane-1,4′-piperidine](90)

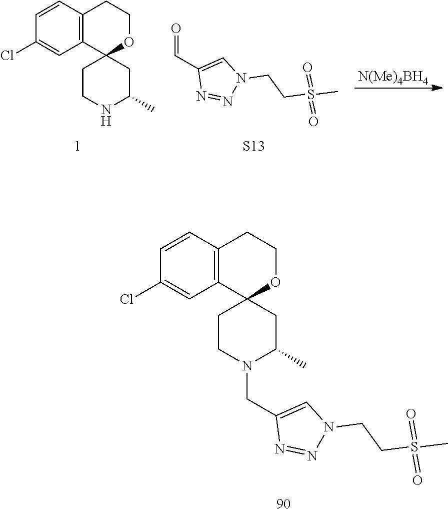

### Reductive Amination Representative Procedure 3

A solution of (2′S)-7-chloro-2′-methyl-spiro[isochromane-1,4′-piperidine](trifluoromethanesulfonic acid) (15 mg, 0.03733 mmol) in DCM (1 mL) was washed with saturated bicarbonate solution on a phase separator (3×2 ml). The organic layer was dried over sodium sulfate, filtered and concentrated in vacuo. The crude was dissolved in DCM (1 mL), added 1-(2-methylsulfonylethyl)triazole-4-carbaldehyde S13 (23 mg, 0.1132 mmol) and (trimethylammonio)methyl cyanoborohydride (100 mg of 1.2 mmol/g, 0.1200 mmol) under nitrogen. Capped and irradiated at 110° C. for 30 minutes. Filtered off the resin and concentrated in vacuo. The crude was purified by reverse phase HPLC (method: Waters XSelect CSH C18 OBD Prep Column; 30×150 mm, 5 micron. gradient: acetonitrile in water with 0.1% trifluoroacetic acid) to afford (1R,2′S)-7-chloro-2′-methyl-1′-[[1-(2-methylsulfonylethyl)triazol-4-yl]methyl]spiro[isochromane-1,4′-piperidine]90 as the trifluoroacetic acid salt (10.3 mg, 63%) LCMS m/z 439.22 [M+H]⁺.

Compounds 91-139 were prepared in a single step using the appropriate intermediate and reagent, and using the reductive amination formation method described for compounds 88-90. Aldehydes were prepared by methods described above or obtained from commercial sources. Any modifications to methods are noted in Table 12 and accompanying footnotes.

TABLE 12
Compounds 91-139

| | | Aldehyde | | ¹HNMR; LCMS m/z |
|---|---|---|---|---|
| Compound | Product | Reagent | Method | [M + H]⁺ |
| 91 | 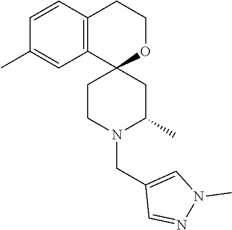 | 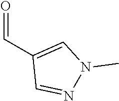 | Cmpd 88 | (400 MHz, DMSO d6), δ ppm 7.56 (s, 1H), 7.29 (s, 1H), 6.99 (s, 1H), 6.95- 6.91 (m, 2H), 3.80 (s, 3H), 3.74-3.68 (m, 3H), 3.42 (d, J =14 Hz, 1H), 2.64 (d, J = 6 Hz, 2H), 2.55-2.50 (m, 1H), 2.45-2.33 (m, 2H), 2.24 (s, 3H), 1.83-1.79 (m, 1H), 1.71 (d, J =13.2 Hz, 2H), 1.64-1.58 (m, 1H), 1.09 (d, J = 6 Hz, |
| | | | | 3H). LCMS m/z |
| | | | | 326.00 [M + H]⁺. |
| 92 | 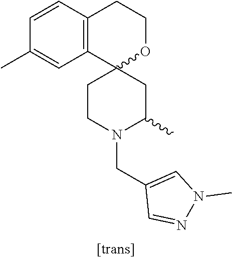 | 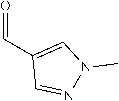 | Cmpd 88¹ | ¹H NMR (300 MHz, Chloroform-d) δ 7.75 (s, 1H), 7.56 (s, 1H), 6.97 (s, 3H), 4.21 (d, J = 14.2 Hz, 1H), 3.99 (d, J = 14.2 Hz, 1H), 3.92 (s, 3H), 3.83 (td, J = 5.7, 2.9 Hz, 2H), 3.21 − 2.88 (m, 3H), 2.73 (q, J = 5.0 Hz, 2H), 2.29 (s, 3H), 2.18 − 1.85 (m, 4H), 1.39 (d, J = 6.4 Hz, 3H). LCMS m/z 326.29 M + H]⁺. |
| 93 | 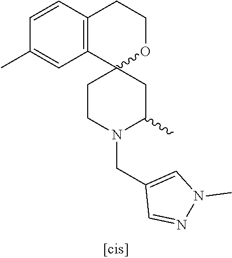 | 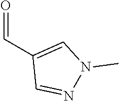 | Cmpd 88² | ¹H NMR (300 MHz, Chloroform-d) δ 7.77 (s, 1H), 7.61 (s, 1H), 6.97 (d, J = 2.3 Hz, 3H), 4.04 (s, 2H), 3.90 (d, J = 6.9 Hz, 5H), 3.53 (s, 1H), 3.31-3.30 (m, 1H), 3.01 (d, J = 12.7 Hz, 1H), 2.90 − 2.66 (m, 2H), 2.35 − 2.14 (m, 5H), 2.02 (dd, J = 35.9, 14.9 Hz, 2H), 1.48 (d, J = 7.0 Hz, 3H). LCMS m/z 326.29 [M + H]⁺. |
| 94 | 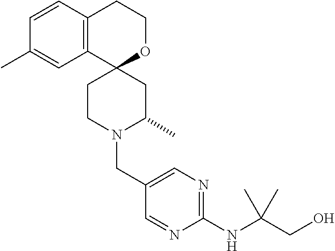 [SMILES: Cc1ccc2c(c1)[C@]1(CCN(Cc3cnc(NC(C)(C)CO)nc3)[C@@H](C)C1)OCC2] | 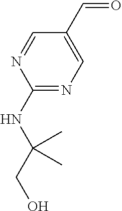 | Cmpd 88 | ¹H NMR (300 MHz, Chloroform-d) δ 8.26 (s, 2H), 6.96 (d, J = 8.2 Hz, 3H), 3.99 (d, J = 13.3 Hz, 1H), 3.83 (d, J = 7.1 Hz, 2H), 3.68 (s, 2H), 3.22 (d, J = 13.7 Hz, 1H), 2.70 (dd, J = 14.2, 8.7 Hz, 3H), 2.49 (s, 1H), 2.28 (s, 3H), 1.95 − 1.70 (m, 5H), 1.39 (s, 6H), 1.26 (d, J = 6.2 Hz, 3H). LCMS m/z 411.27 [M + H]⁺. |
| 95 | 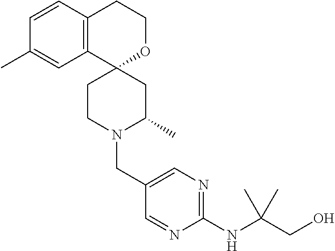 [SMILES: Cc1ccc2c(c1)[C@@]1(CCN(Cc3cnc(NC(C)(C)CO)nc3)[C@@H](C)C1)OCC2] | 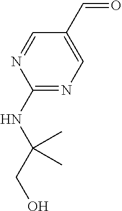 | Cmpd 88 | LCMS m/z 411.27 [M + H]⁺. |
| 96 | 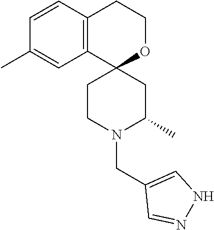 [SMILES: Cc1ccc2c(c1)[C@]1(CCN(Cc3cn[nH]c3)[C@@H](C)C1)OCC2] | 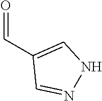 | Cmpd 88 | ¹H NMR (300 MHz, Chloroform-d) δ 8.74 (s, 1H), 7.58 (s, 2H), 7.02 (s, 1H), 6.96 (s, 2H), 4.01 (d, J = 14.3 Hz, 1H), 3.82 (h, J = 6.0 Hz, 2H), 3.69 (d, J = 14.3 Hz, 1H), 2.90 − 2.48 (m, 5H), 2.30 (s, 3H), 2.14 − 1.96 (m, 1H), 1.89 (d, J = 7.2 Hz, 3H), 1.27 (d, J = 6.4 Hz, 3H). LCMS m/z 312.25 [M + H]⁺. |
| 97 | 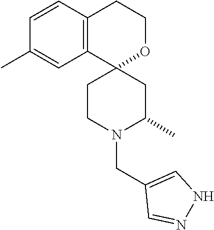 | 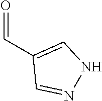 | | ¹H NMR (300 MHz, Chloroform-d) δ 8.82 (s, 1H), 7.65 (s, 2H), 7.01 (s, 1H), 6.96 (s, 2H), 3.87 (t, J = 5.5 Hz, 2H), 3.73 (s, 2H), 3.29 (s, 1H), 3.10 − 2.77 (m, 2H), 2.77 − 2.59 (m, 2H), 2.30 (s, 3H), 2.25 − 2.09 (m, 2H), 2.01 − 1.89 (m, 1H), 1.83 (d, J = 14.0 Hz, 1H), 1.28 (d, J = 7.0 Hz, 3H). LCMS m/z 312.25 [M + H]⁺. |
| 98 | 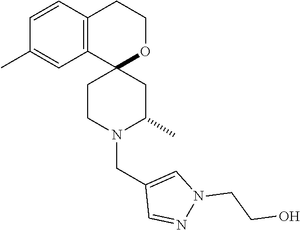 | 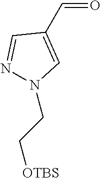 [SMILES: CC(C)(C)[Si](C)(C)OCCn1cc(C=O)cn1] | Cmpd 88³ | ¹H NMR (300 MHz, Chloroform-d) δ 7.47 (s, 1H), 7.38 (s, 1H), 7.01 (s, 1H), 6.96 (s, 2H), 4.22 (dd, J = 5.5, 4.0 Hz, 2H), 4.00 (t, J = 4.8 Hz, 2H), 3.90 − 3.72 (m, 3H), 3.56 (d, J = 14.2 Hz, 1H), 3.21 (br s, 1H), 2.76 (qd, J = 13.9, 12.1, 5.8 Hz, 3H), 2.64 − 2.39 (m, 2H), 2.29 (s, 3H), 2.09 − 1.71 (m, 4H), 1.20 (d, J = 6.2 |
| | | | | Hz, 3H). LCMS m/z |
| | | | | 356.27 [M + H]⁺. |
| 99 | 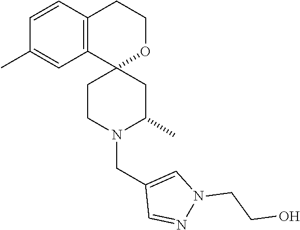 | 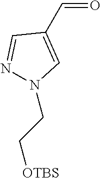 [SMILES: CC(C)(C)[Si](C)(C)OCCn1cc(C=O)cn1] | Cmpd 88⁴ | ¹H NMR (300 MHz, Chloroform-d) δ 7.52 (dt, J = 3.3, 2.1 Hz, 1H), 7.44 (tt, J = 2.0, 1.1 Hz, 1H), 7.01 (d, J = 2.3 Hz, 1H), 6.96 − 6.90 (m, 2H), 4.22 (ddt, J = 8.3, 3.6, 1.7 Hz, 2H), 4.07 − 3.94 (m, 2H), 3.87 (dddd, J = 7.9, 6.4, 2.7, 1.5 Hz, 2H), 3.58 (tt, J = 3.2, 1.6 Hz, 2H), 3.20 (dt, J = 8.2, 3.4 Hz, 1H), 2.98 − 2.76 (m, |
| | | | | 1H), 2.76 − 2.49 (m, |
| | | | | 1H), 2.42 − 2.18 (m, |
| | | | | 8H), 2.18 − 2.00 (m, |
| | | | | 1H), 1.99 − 1.74 (m, |
| | | | | 1H), 1.22 (dddd, J = |
| | | | | 12.6, 6.8, 2.9, 1.6 Hz, |
| | | | | 3H). LCMS m/z |
| | | | | 356.27 [M + H]⁺. |
| 100 | 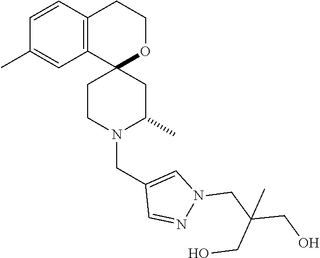 [SMILES: Cc1ccc2c(c1)[C@]1(CCN(Cc3cnn(CC(C)(CO)CO)c3)[C@@H](C)C1)OCC2] | 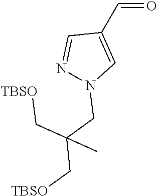 | Cmpd 88⁵ | ¹H NMR (300 MHz, Chloroform-d) δ 7.46 (s, 1H), 7.41 (s, 1H), 7.01 (s, 1H), 6.96 (s, 2H), 4.25 (d, J = 2.9 Hz, 2H), 3.94 (d, J = 14.2 Hz, 1H), 3.82 (q, J = 5.3 Hz, 2H), 3.56 (dd, J = 23.8, 12.8 Hz, 5H), 3.34 (d, J = 10.5 Hz, 2H), 2.86 − 2.47 (m, 4H), 2.30 (s, 3H), 1.89 (d, J = 16.0 Hz, 3H), 1.64 (s, 2H), 1.28 − 1.20 (m, 3H), 0.83 (s, 3H). LCMS m/z 414.25 [M + H]⁺. |
| 101 | 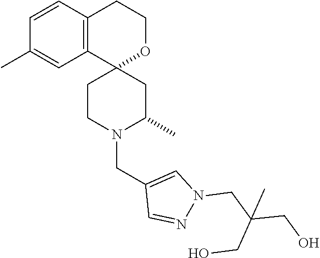 [SMILES: Cc1ccc2c(c1)[C@@]1(CCN(Cc3cnn(CC(C)(CO)CO)c3)[C@@H](C)C1)OCC2] | 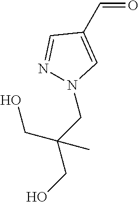 | Cmpd 88⁶ | LCMS m/z 414.25 [M + H]⁺. |
| 102 | 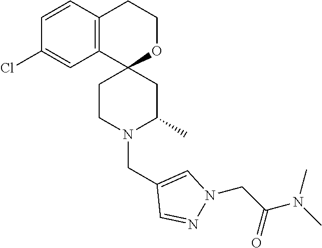 [SMILES: C[C@H]1C[C@@]2(CCN1Cc1cnn(CC(=O)N(C)C)c1)OCCc1ccc(Cl)cc12] | 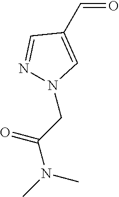 | Cmpd 88 | ¹H NMR (300 MHz, Chloroform-d) δ 7.90 (s, 1H), 7.70 (s, 1H), 7.27 − 7.10 (m, 3H), 5.21 (s, 2H), 4.63 (d, J = 14.1 Hz, 1H), 4.25 (d, J = 13.9 Hz, 1H), 3.90 (q, J = 5.3 Hz, 2H), 3.66 − 3.50 (m, 1H), 3.49 − 3.38 (m, 2H), 3.14 (s, 3H), 2.99 (s, 3H), 2.87 − 2.77 (m, 2H), 2.30 − 1.94 (m, 4H), 1.55 (d, J = 6.4 Hz, 3H). LCMS m/z 417.31 |
| | | | | [M + H]⁺. |
| 103 | 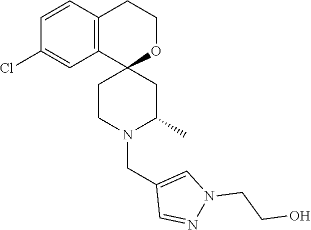 [SMILES: C[C@H]1C[C@@]2(CCN1Cc1cnn(CCO)c1)OCCc1ccc(Cl)cc12] | 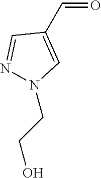 | Cmpd 88 | ¹H NMR (300 MHz, Chloroform-d) δ 7.94 (s, 1H), 7.71 (s, 1H), 7.27 − 7.09 (m, 3H), 4.59 (d, J = 14.0 Hz, 1H), 4.27 (dd, J = 9.9, 4.6 Hz, 3H), 3.89 (dt, J = 10.3, 5.3 Hz, 4H), 3.57 (s, 1H), 3.38 (s, 2H), 2.79 (d, J = 5.4 Hz, 2H), 2.27 − 1.92 (m, 4H), 1.54 (d, J = 6.4 Hz, 3H). LCMS m/z 376.32 [M + H]⁺. |
| 104 | 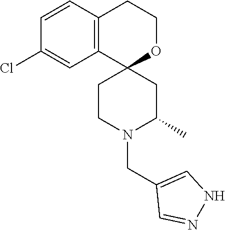 [SMILES: C[C@H]1C[C@@]2(CCN1Cc1cn[nH]c1)OCCc1ccc(Cl)cc12] | 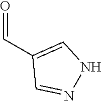 | Cmpd 88 | ¹H NMR (300 MHz, Chloroform-d) δ 7.88 (s, 2H), 7.27 − 7.08 (m, 3H), 4.63 (d, J = 14.0 Hz, 1H), 4.33 (d, J = 14.1 Hz, 1H), 3.88 (q, J = 5.4 Hz, 2H), 3.54 (s, 1H), 3.36 (s, 2H), 2.79 (s, 2H), 2.31 − 1.90 (m, 4H), 1.56 (d, J = 6.5 Hz, 3H). LCMS m/z 332.34 [M + H]⁺. |
| 105 | 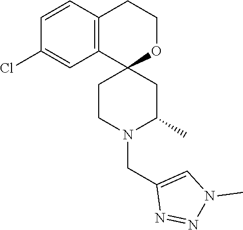 [SMILES: C[C@H]1C[C@@]2(CCN1Cc1cn(C)nn1)OCCc1ccc(Cl)cc12] | 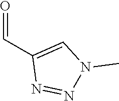 | Cmpd 88 | ¹H NMR (400 MHz, Chloroform-d) δ 8.21 (s, 1H), 7.18 (dt, J = 15.6, 8.4 Hz, 3H), 4.70 (d, J = 14.4 Hz, 1H), 4.57 (d, J = 14.4 Hz, 1H), 4.17 (s, 3H), 3.87 (p, J = 5.9 Hz, 2H), 3.60 (s, 1H), 3.44 (dd, J = 7.3, 4.5 Hz, 2H), 2.79 (d, J = 4.0 Hz, 2H), 2.27 − 1.97 (m, 4H), 1.58 (d, J = 6.4 Hz, 3H). LCMS m/z 347.37 |
| | | | | [M + H]⁺. |
| 106 | 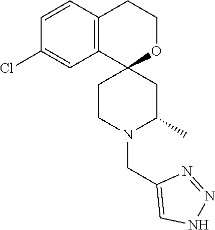 [SMILES: C[C@H]1C[C@@]2(CCN1Cc1c[nH]nn1)OCCc1ccc(Cl)cc12] | 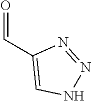 | Cmpd 88 | ¹H NMR (400 MHz, Chloroform-d) δ 7.81 (s, 1H), 7.20 (d, J = 2.1 Hz, 1H), 7.16 − 7.02 (m, 2H), 4.14 (d, J = 14.5 Hz, 1H), 3.96 (d, J = 14.5 Hz, 1H), 3.88 − 3.71 (m, 2H), 2.86 − 2.69 (m, 5H), 2.03 − 1.82 (m, 4H), 1.76 (dd, J = 14.0, 11.3 Hz, 1H), 1.30 (d, J = 6.2 Hz, 3H). LCMS m/z 333.37 [M + H]⁺. |
| 107 | 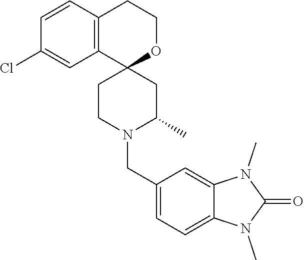 [SMILES: C[C@H]1C[C@@]2(CCN1Cc1ccc3c(c1)n(C)c(=O)n3C)OCCc1ccc(Cl)cc12] | 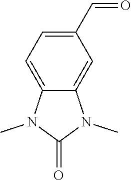 | Cmpd 88 | ¹H NMR (400 MHz, Chloroform-d) δ 7.29 − 6.92 (m, 6H), 4.28 (d, J = 12.9 Hz, 1H), 3.86 (tdd, J = 11.5, 8.9, 5.1 Hz, 2H), 3.46 (s, 3H), 3.44 (s, 3H), 3.31 (d, J = 14.3 Hz, 1H), 2.85 − 2.64 (m, 4H), 2.47 (td, J = 12.0, 3.4 Hz, 1H), 1.87 (dtt, J = 20.0, 13.8, 7.3 Hz, 4H), 1.31 (d, J = 6.2 Hz, 3H). LCMS m/z 426.39 [M + H]⁺. |
| 108 | 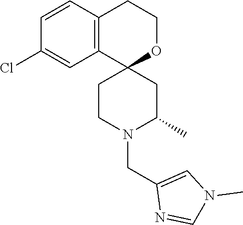 [SMILES: C[C@H]1C[C@@]2(CCN1Cc1cn(C)cn1)OCCc1ccc(Cl)cc12] | 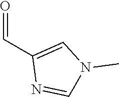 | Cmpd 88 | ¹H NMR (400 MHz, Chloroform-d) δ 7.57 (s, 1H), 7.20 (d, J − 2.1 Hz, 1H), 7.17 − 7.00 (m, 3H), 3.96 − 3.63 (m, 7H), 2.91 − 2.55 (m, 5H), 2.05 − 1.60 (m, 4H), 1.26 (d, J = 6.2 Hz, 3H). LCMS m/z 346.38 [M + H]⁺. |
| 109 | 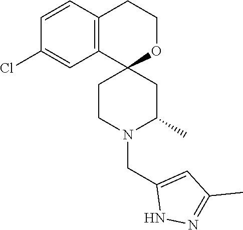 [SMILES: Cc1cc(CN2CC[C@]3(C[C@@H]2C)OCCc2ccc(Cl)cc23)[nH]n1] | 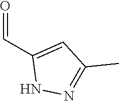 | Cmpd 88 | ¹H NMR (400 MHz, Chloroform-d) δ 7.20 (d, J = 2.0 Hz, 1H), 7.16 − 7.01 (m, 2H), 6.07 (s, 1H), 3.93 (d, J = 14.1 Hz, 1H), 3.87 − 3.77 (m, 2H), 3.64 (d, J = 14.1 Hz, 1H), 2.83 − 2.54 (m, 5H), 2.28 (s, 3H), 2.03 (s, 1H), 1.99 − 1.68 (m, 4H), 1.24 (d, J = 6.2 Hz, 3H). LCMS m/z 346.38 [M + H]⁺. |
| 110 | 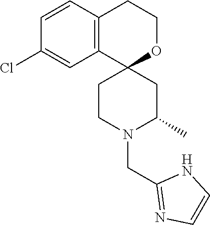 [SMILES: C[C@H]1C[C@@]2(CCN1Cc1ncc[nH]1)OCCc1ccc(Cl)cc12] | 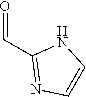 | Cmpd 88 | ¹H NMR (400 MHz, Chloroform-d) δ 7.20 (d, J = 2.1 Hz, 1H), 7.14 − 7.04 (m, 2H), 7.00 (s, 2H), 4.03 (d, J = 14.4 Hz, 1H), 3.90 − 3.77 (m, 2H), 3.61 (d, J = 14.4 Hz, 1H), 2.81 − 2.57 (m, 5H), 1.96 (ddd, J = 13.9, 11.9, 5.2 Hz, 1H), 1.85 (dq, J = 11.6, 2.7 Hz, 2H), 1.73 (dd, J = 13.9, 11.2 Hz, 1H), 1.21 (d, |
| | | | | J = 6.2 Hz, 3H). |
| | | | | LCMS m/z 332.34 |
| | | | | [M + H]⁺. |
| 111 | 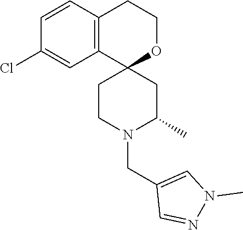 [SMILES: C[C@H]1C[C@@]2(CCN1Cc1cnn(C)c1)OCCc1ccc(Cl)cc12] | 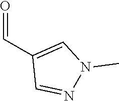 | Cmpd 88 | ¹H NMR (400 MHz, Chloroform-d) δ 7.59 (s, 1H), 7.44 (d, J = 0.9 Hz, 1H), 7.19 (d, J = 2.1 Hz, 1H), 7.15 − 6.95 (m, 2H), 3.89 (s, 3H), 3.84 (d, J = 2.0 Hz, 1H), 3.84 − 3.75 (m, 2H), 3.66 (d, J = 14.2 Hz, 1H), 2.73 (q, J = 5.2 Hz, 3H), 2.68 − 2.55 (m, 2H), 1.98 − 1.81 (m, 3H), 1.72 (dd, J = |
| | | | | 14.1, 11.4 Hz, 1H), |
| | | | | 1.24 (d, J = 6.3 Hz, |
| | | | | 3H). LCMS m/z |
| | | | | 346.38 [M + H]⁺. |
| 112 | 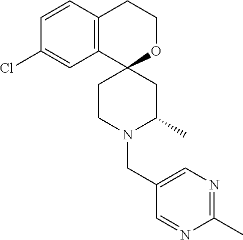 [SMILES: Cc1ncc(CN2CC[C@]3(C[C@@H]2C)OCCc2ccc(Cl)cc23)cn1] | 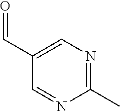 | Cmpd 88 | ¹H NMR (400 MHz, Chloroform-d) δ 8.69 (s, 2H), 7.19 (d, J = 2.1 Hz, 1H), 7.16 − 7.03 (m, 2H), 4.17 (d, J = 13.9 Hz, 1H), 3.95 − 3.78 (m, 2H), 3.34 (s, 1H), 2.85 − 2.72 (m, 3H), 2.69 (s, 3H), 2.62 − 2.56 (m, 1H), 2.51 (td, J = 11.7, 3.5 Hz, 1H), 1.95 − 1.84 (m, 3H), 1.76 (dd, J = 13.8, 11.2 Hz, 1H), 1.24 (d, |
| | | | | J = 6.2 Hz, 3H). |
| | | | | LCMS m/z 358.39 |
| | | | | [M + H]⁺. |
| 113 | 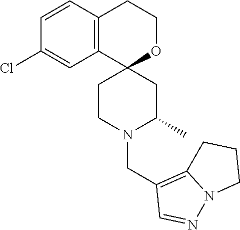 [SMILES: C[C@H]1C[C@@]2(CCN1Cc1cnn3c1CCC3)OCCc1ccc(Cl)cc12] | 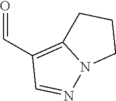 | Cmpd 88 | ¹H NMR (400 MHz, Chloroform-d) δ 7.49 (s, 1H), 7.21 (s, 1H), 7.16 − 7.00 (m, 2H), 4.14 (t, J = 7.2 Hz, 2H), 3.92 − 3.75 (m, 3H), 3.61 (d, J = 14.2 Hz, 1H), 2.96 (h, J = 8.5 Hz, 2H), 2.71 (dtd, J = 34.2, 13.5, 11.6, 7.1 Hz, 7H), 1.95 (dt, J = 21.2, 14.9 Hz, 3H), 1.75 (t, J = 12.7 Hz, 1H), 1.28 (d, J = 6.2 Hz, |
| | | | | 3H). LCMS m/z |
| | | | | 372.38 [M + H]⁺. |
| 114 | 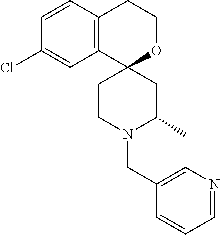 [SMILES: C[C@H]1C[C@@]2(CCN1Cc1cccnc1)OCCc1ccc(Cl)cc12] | 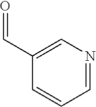 | Cmpd 88 | ¹H NMR (400 MHz, Chloroform-d) δ 8.60 (s, 1H), 8.51 (dd, J = 4.9, 1.6 Hz, 1H), 7.93 (dt, J = 7.8, 1.9 Hz, 1H), 7.51 − 7.43 (m, 1H), 7.21 (s, 1H), 7.17 − 7.06 (m, 2H), 4.34 (d, J = 13.6 Hz, 1H), 3.98 − 3.81 (m, 2H), 3.47 (d, J = 13.7 Hz, 1H), 2.97 − 2.52 (m, 5H), 2.02 − 1.74 (m, 4H), 1.33 (d, J = 6.2 Hz, 3H). LCMS |
| | | | | m/z 343.35 [M + H]⁺. |
| 115 | 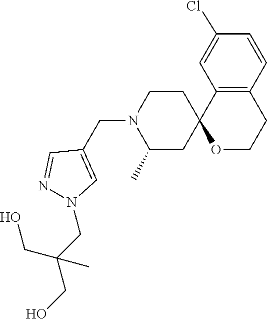 [SMILES: C[C@H]1C[C@@]2(CCN1Cc1cnn(CC(C)(CO)CO)c1)OCCc1ccc(Cl)cc12] | 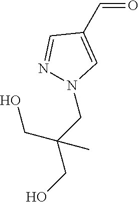 | Cmpd 88 | ¹H NMR (400 MHz, Chloroform-d) δ 7.66 (s, 1H), 7.48 (s, 1H), 7.20 (d, J = 2.1 Hz, 1H), 7.13 (dd, J = 8.2, 2.0 Hz, 1H), 7.07 (d, J = 8.2 Hz, 1H), 4.14 (s, 2H), 3.90 (s, 1H), 3.83 − 3.66 (m, 3H), 3.46 − 3.33 (m, 4H), 2.71 (dtt, J = 23.4, 11.7, 4.8 Hz, 5H), 1.92 (tt, J = 20.8, 8.5 Hz, 3H), 1.74 (dd, J = 14.2, 11.4 Hz, 1H), 1.26 (d, J = 6.2 Hz, 3H), 0.83 (s, 3H). LCMS m/z 434.38 [M + H]⁺. |
| 116 | 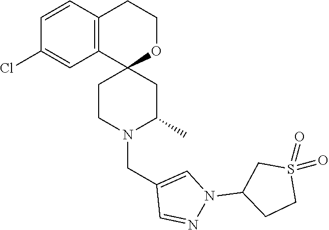 [SMILES: C[C@H]1C[C@@]2(CCN1Cc1cnn(C3CCS(=O)(=O)C3)c1)OCCc1ccc(Cl)cc12] | 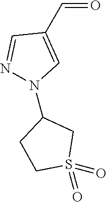 [SMILES: O=Cc1cnn(C2CCS(=O)(=O)C2)c1] | Cmpd 88 | ¹H NMR (400 MHz, Chloroform-d) δ 7.80 (s, 1H), 7.58 (s, 1H), 7.22 (d, J = 2.1 Hz, 1H), 7.20 − 7.04 (m, 2H), 5.36 − 5.16 (m, 1H), 3.96 − 3.89 (m, 1H), 3.87 − 3.79 (m, 2H), 3.72 − 3.63 (m, 2H), 3.61 − 3.53 (m, 1H), 3.47 (td, J = 8.5, 4.4 Hz, 1H), 3.29 − 3.22 (m, 2H), 2.82 − 2.73 (m, 3H), 2.72 − 2.57 (m, 3H), 2.02 − |
| | | | | 1.83 (m, 3H), 1.80 − |
| | | | | 1.70 (m, 1H), 1.27 (d, |
| | | | | J = 6.3 Hz, 3H). |
| | | | | LCMS m/z 450.32 |
| | | | | [M + H]⁺. |
| 117 | 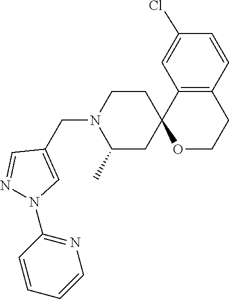 [SMILES: C[C@H]1C[C@@]2(CCN1Cc1cnn(-c3ccccn3)c1)OCCc1ccc(Cl)cc12] | 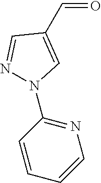 [SMILES: O=Cc1cnn(-c2ccccn2)c1] | Cmpd 88 | ¹H NMR (400 MHz, Chloroform-d) δ 8.58 (s, 1H), 8.44 (d, J = 4.9 Hz, 1H), 7.98 − 7.89 (m, 2H), 7.76 (s, 1H), 7.30 (q, J = 4.5 Hz, 1H), 7.21 (d, J = 2.0 Hz, 1H), 7.15 − 7.00 (m, 2H), 3.99 (d, J = 14.3 Hz, 1H), 3.90 − 3.80 (m, 2H), 3.75 (d, J = 14.3 Hz, 1H), 2.86 − 2.56 (m, 5H), 2.00 − 1.83 (m, 3H), 1.81 − 1.68 (m, 1H), 1.29 (d, J = 6.2 Hz, 3H). LCMS m/z 409.37 [M + H]⁺. |
| 118 | 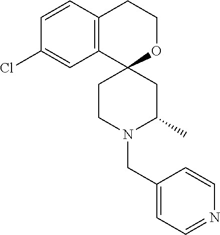 [SMILES: C[C@H]1C[C@@]2(CCN1Cc1ccncc1)OCCc1ccc(Cl)cc12] | 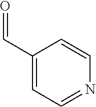 | Cmpd 88 | ¹H NMR (400 MHz, DMSO-d6) δ 8.73 (d, J = 5.0 Hz, 2H), 7.61 (d, J = 5.0 Hz, 2H), 7.33 − 7.26 (m, 1H), 7.23 − 7.12 (m, 2H), 4.89 − 4.75 (m, 1H), 4.29 − 4.15 (m, 1H), 3.92 − 3.80 (m, 2H), 3.09 (qd, J = 7.3, 4.6 Hz, 3H), 2.75 (q, J = 5.3 Hz, 2H), 2.25 − 2.09 (m, 3H), 2.00 (d, J = 15.8 Hz, 1H), 1.48 (d, J = 6.3 Hz, |
| | | | | 3H). LCMS m/z |
| | | | | 343.35 [M + H]⁺. |
| 119 | 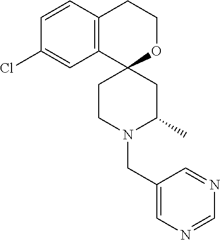 [SMILES: C[C@H]1C[C@@]2(CCN1Cc1cncnc1)OCCc1ccc(Cl)cc12] | 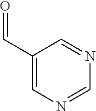 | Cmpd 88 | ¹H NMR (400 MHz, DMSO) δ 8.73 (d, J = 10.0 Hz, 1H), 8.28 (s, 2H), 7.35 − 7.26 (m, 1H), 7.25 − 7.12 (m, 2H), 3.99 − 3.76 (m, 2H), 3.32 − 3.20 (m, 2H), 3.17 − 3.00 (m, 3H), 2.82 − 2.67 (m, 2H), 2.13 − 1.83 (m, 4H), 1.23 (d, J = 6.4 Hz, 3H). LCMS m/z 344.35 [M + H]⁺. |
| 120 | 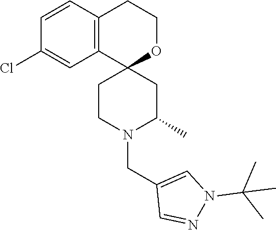 [SMILES: C[C@H]1C[C@@]2(CCN1Cc1cnn(C(C)(C)C)c1)OCCc1ccc(Cl)cc12] | 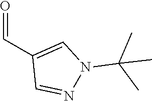 | Cmpd 88 | ¹H NMR (400 MHz, DMSO-d6) δ 8.06 (s, 1H), 7.65 (s, 1H), 7.34 − 7.24 (m, 1H), 7.21 (s, 1H), 7.19 − 7.10 (m, 1H), 4.48 (d, J = 13.8 Hz, 1H), 4.16 (dd, J = 13.8, 5.8 Hz, 1H), 3.91 − 3.68 (m, 2H), 3.32 − 3.17 (m, 2H), 3.18 − 2.98 (m, 1H), 2.74 (s, 2H), 2.09 (d, J = 14.1 Hz, 4H), 1.53 (s, 9H), |
| | | | | 1.44 (d, J = 6.3 Hz, |
| | | | | 3H). LCMS m/z |
| | | | | 388.41 [M + H]⁺. |
| 121 | 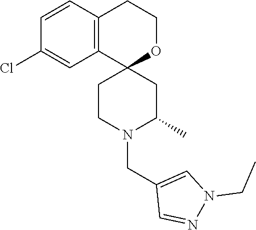 [SMILES: CCn1cc(CN2CC[C@]3(C[C@@H]2C)OCCc2ccc(Cl)cc23)cn1] | 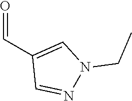 | Cmpd 88 | ¹H NMR (400 MHz, DMSO-d6) δ 7.98 (s, 1H), 7.63 (d, J = 4.2 Hz, 1H), 7.27 (s, 1H), 7.26 − 7.10 (m, 2H), 4.45 (d, J = 13.6 Hz, 1H), 4.34 − 4.09 (m, 3H), 3.94 − 3.76 (m, 2H), 3.37 − 3.24 (m, 2H), 3.17 − 3.01 (m, 1H), 2.79 − 2.66 (m, 2H), 2.28 − 1.96 (m, 4H), 1.56 − 1.31 (m, 6H). LCMS m/z 360.37 |
| | | | | [M + H]⁺. |
| 122 | 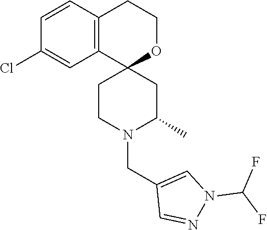 [SMILES: C[C@H]1C[C@@]2(CCN1Cc1cnn(C(F)F)c1)OCCc1ccc(Cl)cc12] | 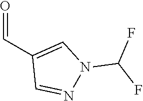 | Cmpd 88 | ¹H NMR (400 MHz, DMSO) δ 8.51 (s, 1H), 8.14 − 7.65 (m, 2H), 7.27 (d, J = 7.9 Hz, 1H), 7.18 (d, J = 20.5 Hz, 2H), 4.62 − 4.53 (m, 1H), 4.34 − 4.19 (m, 1H), 3.93 − 3.66 (m, 2H), 3.31 − 3.18 (m, 1H), 3.13 − 3.00 (m, 2H), 2.81 − 2.64 (m, 2H), 2.24 − 1.97 (m, 4H), 1.44 (d, J = 6.3 Hz, 3H). LCMS m/z 382.32 |
| | | | | [M + H]⁺. |
| 123 | 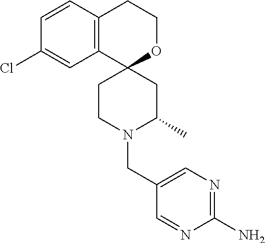 [SMILES: C[C@H]1C[C@@]2(CCN1Cc1cnc(N)nc1)OCCc1ccc(Cl)cc12] | 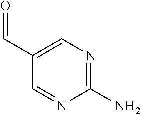 | Cmpd 88 | ¹H NMR (400 MHz, DMSO) δ 8.38 (d, J = 4.0 Hz, 2H), 7.27 (s, 1H), 7.25 − 6.95 (m, 2H), 4.01 (s, 2H), 3.54 − 3.36 (m, 2H), 3.27 − 2.96 (m, 3H), 2.76 (s, 2H), 2.27 − 1.90 (m, 4H), 1.18 (s, 3H). LCMS m/z 359.34 [M + H]⁺. |
| 124 | 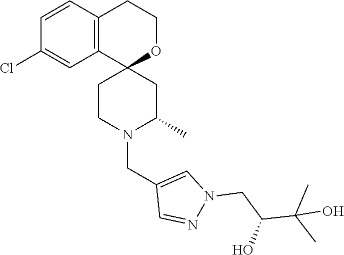 [SMILES: C[C@H]1C[C@@]2(CCN1Cc1cnn(C[C@@H](O)C(C)(C)O)c1)OCCc1ccc(Cl)cc12] | 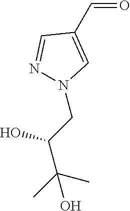 | Cmpd 88 | ¹H NMR (300 MHz, Chloroform-d) δ 7.93 (s, 1H), 7.70 (s, 1H), 7.26 − 7.07 (m, 3H), 4.66 − 4.41 (m, 2H), 4.29 (d, J = 14.1 Hz, 1H), 4.08 (dd, J = 13.9, 9.9 Hz, 1H), 3.94 − 3.83 (m, 2H), 3.71 (dd, J = 9.9, 2.2 Hz, 1H), 3.59 − 3.51 (m, 1H), 3.44 − 3.33 (m, 2H), 2.87 − 2.72 (m, 2H), 2.34 − 1.89 (m, 4H), 1.55 (d, J = 6.4 Hz, 3H), 1.26 (s, |
| | | | | 3H), 1.23 (s, 3H). |
| | | | | LCMS m/z 434.38 |
| | | | | [M + H]⁺. |
| 125 | 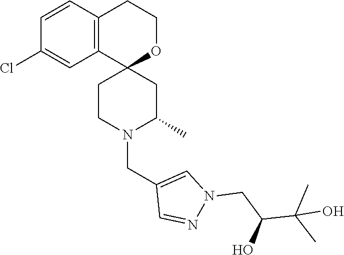 [SMILES: C[C@H]1C[C@@]2(CCN1Cc1cnn(C[C@H](O)C(C)(C)O)c1)OCCc1ccc(Cl)cc12] | 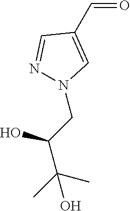 | Cmpd 88 | ¹H NMR (300 MHz, Chloroform-d) δ 7.95 (s, 1H), 7.71 (s, 1H), 7.27 − 7.10 (m, 3H), 4.63 − 4.41 (m, 2H), 4.30 (d, J = 14.1 Hz, 1H), 4.10 (dd, J = 13.9, 9.9 Hz, 1H), 3.97 − 3.82 (m, 2H), 3.73 (dd, J = 9.9, 2.3 Hz, 1H), 3.64 − 3.51 (m, 1H), 3.45 − 3.35 (m, 2H), 2.80 (dd, J = 6.3, 4.1 Hz, 2H), 2.29 − 1.98 (m, 4H), 1.55 (d, J = 6.4 Hz, 3H), |
| | | | | 1.27 (s, 3H), 1.25 (s, |
| | | | | 3H). LCMS m/z |
| | | | | 434.38 [M + H]⁺. |
| 126 | 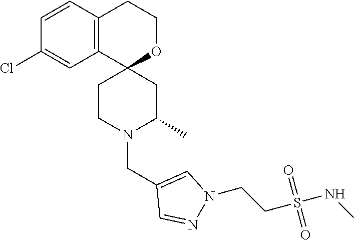 [SMILES: CNS(=O)(=O)CCn1cc(CN2CC[C@]3(C[C@@H]2C)OCCc2ccc(Cl)cc23)cn1] | 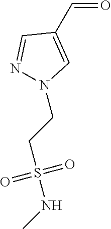 | Cmpd 88 | ¹H NMR (300 MHz, Chloroform-d) δ 8.01 (s, 1H), 7.74 (s, 1H), 7.30 − 6.98 (m, 3H), 4.66 − 4.55 (m, 2H), 4.52 (s, 1H), 4.28 (d, J = 14.1 Hz, 1H), 3.95 − 3.76 (m, 2H), 3.59 (t, J = 6.4 Hz, 3H), 3.34 (s, 2H), 2.79 (d, J = 5.3 Hz, 2H), 2.67 (s, 3H), 2.28 − 2.05 (m, 4H), 1.55 (d, J = 6.4 Hz, 3H). LCMS m/z 453.3 |
| | | | | [M + H]⁺. |
| 127 | 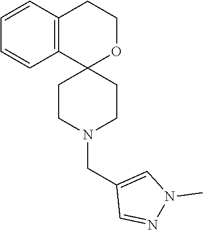 [SMILES: Cn1cc(CN2CCC3(CC2)OCCc2ccccc23)cn1] | 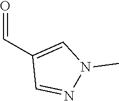 | Cmpd 89 | ¹H NMR (400 MHz, Chloroform-d) δ 7.46 (s, 1H), 7.34 (s, 1H), 7.25 − 7.13 (m, 3H), 7.13 − 7.07 (m, 1H), 3.94 − 3.85 (m, 5H), 3.51 (s, 2H), 2.88 − 2.74 (m, 4H), 2.41 (ddd, J = 12.6, 11.2, 2.6 Hz, 2H), 2.05 (td, J = 13.3, 4.5 Hz, 2H), 1.98 − 1.84 (m, 2H). LCMS m/z 297.40 [M + H]⁺. |
| 128 | 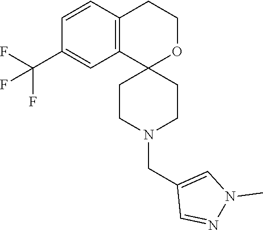 [SMILES: Cn1cc(CN2CCC3(CC2)OCCc2ccc(C(F)(F)F)cc23)cn1] | 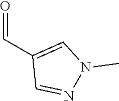 | Cmpd 89 | LCMS m/z 366.10 [M + H]⁺. |
| 129 | 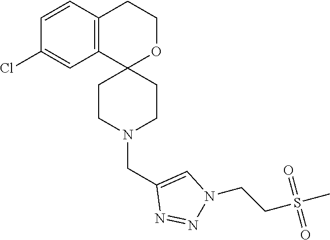 [SMILES: CS(=O)(=O)CCn1cc(CN2CCC3(CC2)OCCc2ccc(Cl)cc23)nn1] | 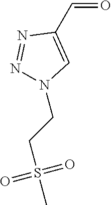 | Cmpd 90⁷ | ¹H NMR (300 MHz, Chloroform-d) δ 8.27 (s, 1H), 7.17 (dd, J = 8.1, 2.1 Hz, 1H), 7.11 − 7.03 (m, 2H), 4.95 (t, J = 6.7 Hz, 2H), 4.42 (s, 2H), 3.88 (t, J = 5.4 Hz, 2H), 3.72 (t, J = 6.7 Hz, 2H), 3.52 (d, J = 11.0 Hz, 2H), 3.35 (t, J = 12.3 Hz, 2H), 2.94 (s, 3H), 2.81 (t, J = 5.5 Hz, 2H), 2.36 (td, J = 13.8, 3.8 Hz, 2H), |
| | | | | 2.08 (d, J = 14.8 Hz, |
| | | | | 2H). LCMS m/z |
| | | | | 425.19 [M + H]⁺. |
| 130 | 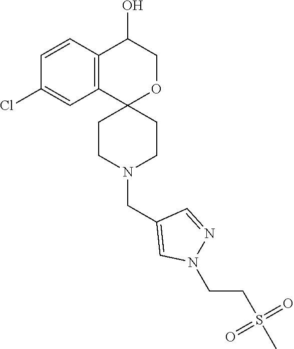 [SMILES: CS(=O)(=O)CCn1cc(CN2CCC3(CC2)OCC(O)c2ccc(Cl)cc23)cn1] | 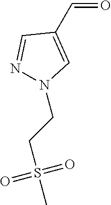 | Cmpd 90 | ¹H NMR (400 MHz, Chloroform-d) δ 7.63 − 7.53 (m, 2H), 7.31 − 7.26 (m, 1H), 7.22 − 7.09 (m, 2H), 5.29 − 5.21 (m, 1H), 4.63 (t, J = 6.1 Hz, 2H), 3.91 (dd, J = 11.7, 3.4 Hz, 1H), 3.74 (dd, J = 11.6, 5.6 Hz, 1H), 3.70 − 3.60 (m, 4H), 2.92 (s, 2H), 2.51 (s, 2H), 2.19 (s, 2H), 2.01 (s, 2H), 1.77 (d, J = 13.5 Hz, 3H). LCMS m/z 440.21 [M + H]⁺. |
| 131 | 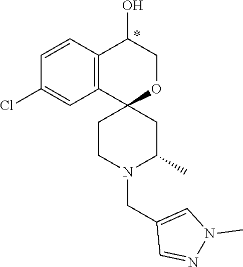 | 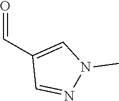 | Cmpd 88 | ¹H NMR (300 MHz, Chloroform-d) δ 7.91 (s, 1H), 7.73 − 7.63 (m, 1H), 7.48 (d, J = 8.3 Hz, 1H), 7.34 (s, 1H), 7.20 (s, 1H), 4.60 − 4.50 (m, 2H), 4.31 (d, J = 14.1 Hz, 1H), 4.02 − 3.87 (m, 4H), 3.78 − 3.67 (m, 1H), 3.62 − 3.48 (m, 1H), 3.43 − 3.35 (m, 2H), 2.34 − 1.80 (m, 4H), 1.55 (d, 3H). LCMS m/z 362.32 [M + H]⁺. |
| 132 | 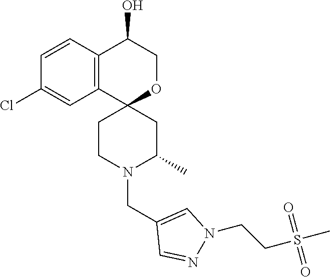 [SMILES: C[C@H]1C[C@@]2(CCN1Cc1cnn(CCS(C)(=O)=O)c1)OC[C@H](O)c1ccc(Cl)cc12] | 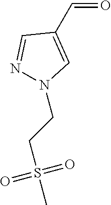 | Cmpd 88 | ¹H NMR (300 MHz, Chloroform-d) δ 8.02 (s, 1H), 7.75 (s, 1H), 7.47 (d, J = 8.1 Hz, 1H), 7.33 (dd, J = 8.2, 2.1 Hz, 1H), 7.20 (s, 1H), 4.71 (t, J = 6.2 Hz, 2H), 4.64 − 4.48 (m, 2H), 4.32 (d, J = 14.1 Hz, 1H), 4.07 − 3.86 (m, 1H), 3.84 − 3.61 (m, 3H), 3.61 − 3.42 (m, 1H), 3.43 − 3.37 (m, 2H), 2.87 (s, 3H), 2.25 (d, J = 17.1 Hz, 3H), 2.13 − 1.86 (m, 1H), 1.54 (d, J = |
| | | | | 6.3 Hz, 3H). LCMS |
| | | | | m/z 454.21 [M + H]⁺. |
| 133 | 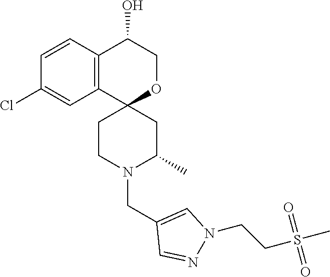 [SMILES: C[C@H]1C[C@@]2(CCN1Cc1cnn(CCS(C)(=O)=O)c1)OC[C@@H](O)c1ccc(Cl)cc12] | 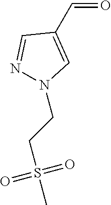 | Cmpd 88 | ¹H NMR (300 MHz, Chloroform-d) δ 8.02 (s, 1H), 7.75 (s, 1H), 7.45 (d, J = 8.1 Hz, 1H), 7.32 (dd, J = 8.3, 2.1 Hz, 1H), 7.21 (d, J = 1.9 Hz, 1H), 4.71 (t, J = 6.2 Hz, 2H), 4.55 (d, J = 14.7 Hz, 2H), 4.32 (d, J = 14.1 Hz, 1H), 3.91 (dd, J = 12.1, 3.4 Hz, 1H), 3.75 (dt, J = 12.3, 5.3 Hz, 3H), 3.56 (d, J = 9.6 Hz, 1H), 3.45 − 3.33 (m, 2H), 2.86 (s, 3H), 2.42 − 1.96 (m, 4H), 1.55 (d, J = 6.4 |
| | | | | Hz, 3H). LCMS m/z |
| | | | | 454.25 [M + H]⁺. |
| 134 | 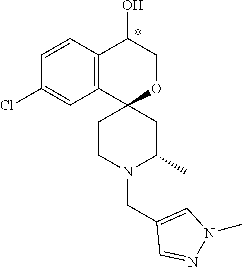 | 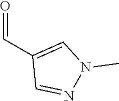 | Cmpd 88 | ¹H NMR (300 MHz, Chloroform-d) δ 7.89 (s, 1H), 7.71 − 7.62 (m, 1H), 7.46 (d, J = 8.3 Hz, 1H), 7.36 − 7.27 (m, 1H), 7.19 (s, 1H), 4.62 − 4.46 (m, 2H), 4.30 (d, J = 14.1 Hz, 1H), 3.95 (s, 3H), 3.90 (d, J = 3.3 Hz, 1H), 3.76 (dd, J = 12.1, 4.6 Hz, 1H), 3.67 − 3.48 (m, 1H), 3.43 − 3.33 (m, 2H), 2.35 − 1.96 (m, 4H), 1.55 (d, J = 6.4 Hz, 3H). LCMS m/z |
| | | | | 362.32 [M + H]⁺. |
| 135 | 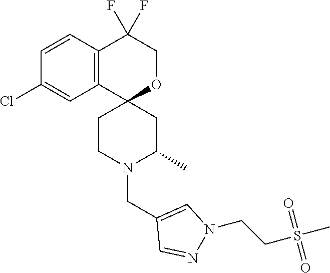 [SMILES: C[C@H]1C[C@@]2(CCN1Cc1cnn(CCS(C)(=O)=O)c1)OCC(F)(F)c1ccc(Cl)cc12] | 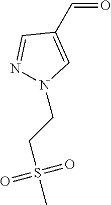 | Cmpd 88 | ¹H NMR (300 MHz, Chloroform-d) δ 7.99 (s, 1H), 7.80 − 7.64 (m, 2H), 7.49 (d, J = 8.5 Hz, 1H), 7.41 (s, 1H), 4.71 (t, J = 6.2 Hz, 2H), 4.47 (d, J = 14.1 Hz, 1H), 4.35 − 4.22 (m, 1H), 4.09 (t, J = 10.6 Hz, 2H), 3.73 (t, J = 6.3 Hz, 2H), 3.46 (s, 1H), 3.27 − 3.08 (m, 3H), 2.87 (s, 3H), 2.37 − 1.98 (m, 4H), 1.53 (d, J = 6.4 Hz, 3H). LCMS m/z 474.21 |
| | | | | [M + H]⁺. |
| 136 | 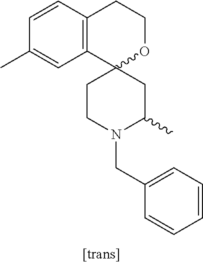 [SMILES: Cc1ccc2c(c1)C1(CCN(Cc3ccccc3)C(C)C1)OCC2] | 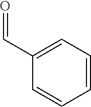 | Cmpd 88 | ¹H NMR (300 MHz, Chloroform-d) δ 7.52 (dd, J = 6.9, 3.7 Hz, 5H), 7.01 (s, 2H), 6.92 (s, 1H), 4.85 (d, J = 13.1 Hz, 1H), 4.13 (d, J = 13.1 Hz, 1H), 3.96 − 3.81 (m, 2H), 3.74 (dt, J = 11.7, 6.1 Hz, 1H), 3.25 − 3.35 (m, 1H), 3.18 (dt, J = 12.4, 3.5 Hz, 1H), 2.77 (q, J = 5.3 Hz, 2H), 2.29 (s, 3H), 2.18 (d, J = 9.9 Hz, 2H), 2.09 (dd, J = |
| | | | | 11.5, 4.1 Hz, 2H), |
| | | | | 1.61 (d, J = 6.5 Hz, |
| | | | | 3H). LCMS m/z |
| | | | | 322.31 [M + H]⁺. |
| 137 | 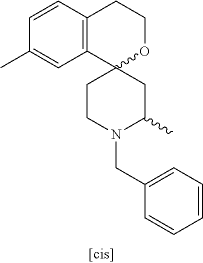 [SMILES: Cc1ccc2c(c1)C1(CCN(Cc3ccccc3)C(C)C1)OCC2] | 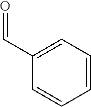 | Cmpd 88 | ¹H NMR (300 MHz, Chloroform-d) δ 7.61 (dd, J = 6.6, 2.9 Hz, 2H), 7.52 (q, J = 3.4, 2.8 Hz, 3H), 7.02 (d, J = 10.4 Hz, 2H), 6.91 (s, 1H), 4.44 − 4.28 (m, 2H), 3.92 (dd, J = 6.5, 4.6 Hz, 2H), 3.79 (q, J = 7.0 Hz, 1H), 3.70 − 3.55 (m, 1H), 3.30 − 3.15 (m, 1H), 2.79 (dt, J = 9.9, 5.5 Hz, 2H), 2.44 − 2.32 (m, 2H), 2.25 (d, J = 13.3 Hz, 4H), |
| | | | | 2.14 − 2.00 (m, 1H), |
| | | | | 1.68 (d, J = 7.2 Hz, |
| | | | | 3H). LCMS m/z |
| | | | | 322.20 [M + H]⁺. |
| 138 | 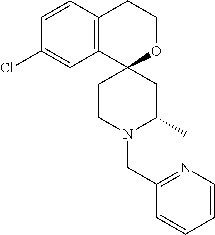 [SMILES: C[C@H]1C[C@@]2(CCN1Cc1ccccn1)OCCc1ccc(Cl)cc12] | 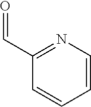 | Cmpd 88 | ¹H NMR (400 MHz, DMSO-d6) δ 8.73 (d, J = 4.8 Hz, 1H), 7.96 (td, J = 7.7, 1.8 Hz, 1H), 7.60 (d, J = 7.8 Hz, 1H), 7.51 (dd, J = 7.7, 4.8 Hz, 1H), 7.28 (dd, J = 8.3, 2.0 Hz, 2H), 7.20 (d, J = 8.3 Hz, 1H), 4.84 (d, J = 14.3 Hz, 1H), 4.46 (d, J = 14.2 Hz, 1H), 3.85 (qt, J = 12.1, 5.7 Hz, 2H), 3.10 (tt, J = 12.0, 6.2 Hz, 3H), |
| | | | | 2.75 (q, J = 5.0 Hz, |
| | | | | 2H), 2.38 − 2.16 (m, |
| | | | | 2H), 2.09 (d, J = 15.0 |
| | | | | Hz, 1H), 1.99 (d, J = |
| | | | | 14.7 Hz, 1H), 1.38 (d, |
| | | | | J = 6.4 Hz, 3H). |
| | | | | LCMS m/z 343.35 |
| | | | | [M + H]⁺. |
| 139 | 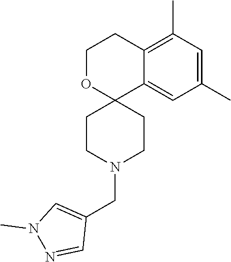 | 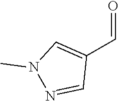 | Cmpd 88 | ¹H NMR (400 MHz, DMSO-d6) δ 7.59 (bs, 1H), 7.33 (bs, 1H), 7.09-7.00 (m, 3H), 3.83-3.80 (m, 5H), 3.32(s, 2H), 2.59-2.55 (m, 4H), 2.32-2.29 (m, 2H), 2.17(s, 3H), 1.92- 1.89(m, 2H), 1.80- 1.78 (m, 2H). LCMS m/z 312.0 [M + H]⁺. |

¹DCM (.8 ml) was used at room temperature
²DCM (.8 ml) was used at room temperature, racemic 7-chloro-2′-methylspiro[isochromane-1,4′-piperidine] was used.
³DCM (0.8 ml) used at room temperature Silyl ether was deprotected; stirred in 36 ul HCl ((37% v/v) and 1 ml MeOH for 1 hour.
⁴DCM (0.8 ml) used at room temperature Silyl ether was deprotected; stirred in 36 ul HCl ((37% v/v) and 1 ml MeOH for 1 hour.
⁵DCM (0.8 ml) used at room temperature Silyl ether was deprotected; stirred in 71 ul HCl ((37% v/v) and 1 ml MeOH for 1 hour.
⁶DCM (0.8 ml) used at room temperature Silyl ether was deprotected; stirred in 71 ul HCl ((37% v/v) and 1 ml MeOH for 1 hour.
⁷60 ul of acetic acid was added to the reaction

### Compound 140

### 7-chloro-3′-methyl-1′-[(1-methylpyrazol-4-yl)methyl]spiro[isochromane-1,4′-piperidine]

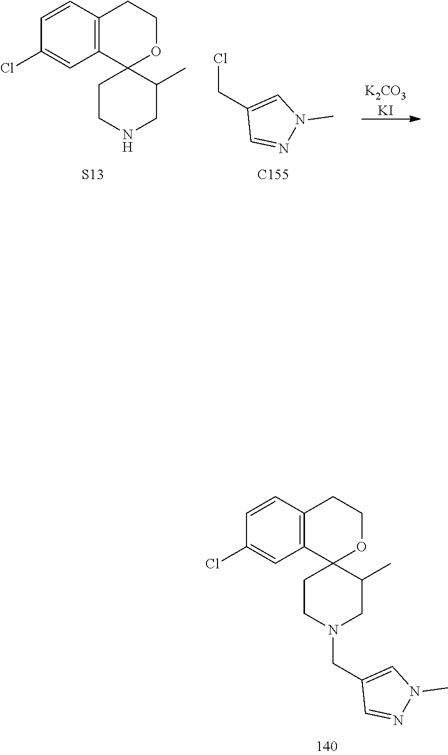

### Synthesis of 7-chloro-3′-methyl-1′-[(1-methylpyrazol-4-yl)methyl]spiro[isochromane-1,4′-piperidine](140)

To a stirred solution of 7-chloro-3′-methyl-spiro[isochromane-1,4′-piperidine]S13 (110 mg, 419.46 μmol) in DMF (1.1000 mL) was added 4-(chloromethyl)-1-methyl-pyrazole C155 (32.863 mg, 251.68 μmol), KI (41.780 mg, 251.68 μmol) and K₂CO₃ (179.67 mg, 0.0013 mol) at room temperature. The reaction was heated to 80° C. for 16 h. The reaction was cooled to room temperature and quenched with water (20 ml) and extracted with ethyl acetate (2×50 ml). The combined organic layers were dried over sodium sulfate, filtered and concentrated under reduced pressure. The crude was purified using reverse HPLC (Method: Waters XSelect CSH C18 OBD Prep Column; 30×150 mm, 5 micron. gradient: acetonitrile in water with 0.1% trifluoroacetic acid) to afford as the trifluoroacetic acid salt 7-chloro-3′-methyl-1′-[(1-methylpyrazol-4-yl)methyl]spiro[isochromane-1,4′-piperidine]140 (18 mg, 9%) (400 MHz, DMSO d6), δ ppm 7.88 (s, 1H), 7.59 (s, 1H), 7.29-7.27 (m, 1H), 7.23-7.20 (m, 1H), 7.05 (s, 1H), 4.26 (s, 2H), 3.98-3.94 (m, 1H), 3.87 (s, 3H), 3.71 (t, J=9.2 Hz, 1H), 3.28-3.23 (m, 2H), 3.09-3.03 (m, 1H), 2.92-2.78 (m, 2H), 2.63-2.51 (m, 1H), 2.33-2.29 (m, 2H), 2.02-1.98 (m, 1H), 0.55 (d, J=6.8 Hz, 3H). [LCMS m/z 346.48 [M+H]⁺.

### Preparation S19

### N-diazo-1,1,1-trifluoro-methanesulfonamide (S19)

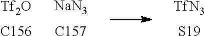

### Synthesis of N-diazo-1,1,1-trifluoro-methanesulfonamide (S19)

To a mixture of sodium azide C157 (300 mg, 4.615 mmol) in water (1 mL) was added DCM (2 mL), and the biphasic mixture was cooled to 0° C. To this mixture was added Tf₂O C156 (380 μL, 2.259 mmol), stirred for 2 hours. At this time, the layers were separated and the aqueous layer was extracted with an additional layer of DCM (2 mL). The combined organic layer was washed with sat. sodium bicarbonate (1 mL). The organic layer was added to a volumetric flask and diluted with additional DCM up to 5 mL to create a stock solution that was used the same day (0.452 M) and the reaction was not further characterized.

### Compound 141

### 2-[4-[[(1R,2′S)-7-chloro-2′-methyl-spiro[isochromane-1,4′-piperidine]-1′-yl]methyl]triazol-1-yl]ethanol (141)

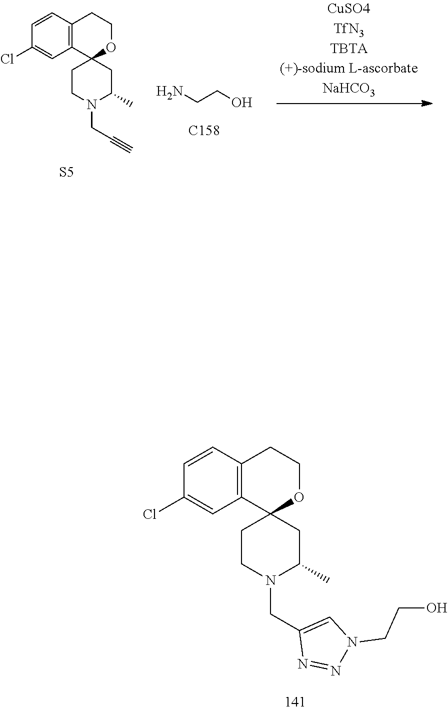

### Synthesis of 2-[4-[[(R,2′S)-7-chloro-2′-methyl-spiro[isochromane-1,4′-piperidine]-1′-yl]methyl]triazol-1-yl]ethanol (141)

Representative Procedure for Azido Transfer Click. Method A

The substrate 2-aminoethanol C158 (8.348 mg, 0.1367 mmol), CuSO₄ (0.2755 mg, 0.07646 μL, 0.001726 mmol) and NaHCO₃ (6.175 mg, 0.07351 mmol) were suspended in the same volume of water (380 μL) as the volume of the TfN₃ solution to be employed. Then TfN₃ (370 μL of 0.452 M, 0.1672 mmol) solution was added, followed by addition of methanol (700 μL) until the solution became homogeneous. The reaction was stirred at room temperature for 1 hour. (1R,2′S)-7-chloro-2′-methyl-1′-prop-2-ynyl-spiro[isochromane-1,4′-piperidine]S5 (10 mg, 0.03451 mmol), TBTA (0.9159 mg, 0.001726 mmol) and (+)-sodium L-ascorbate (6.837 mg, 0.03451 mmol) were added and the reaction was heated at 50° C. for 2 hours. Concentrated the reaction and purified by reverse phase HPLC (Method: Waters XSelect CSH C18 OBD Prep Column; 30×150 mm, 5 micron. gradient: acetonitrile in water with 0.1% trifluoroacetic acid) to afford as the trifluoroacetic acid salt 2-[4-[[(1R,2′S)-7-chloro-2′-methyl-spiro[isochromane-1,4′-piperidine]-1′-yl]methyl]triazol-1-yl]ethanol 141 (10.7 mg, 58%) ¹H NMR (400 MHz, Chloroform-d) δ 8.28 (s, 1H), 7.29-7.07 (m, 3H), 4.74 (d, J=14.4 Hz, 1H), 4.64-4.48 (m, 3H), 4.05-3.93 (m, 2H), 3.88 (q, J=6.0 Hz, 2H), 3.64 (dt, J=9.6, 3.5 Hz, 1H), 3.47 (dd, J=7.3, 4.5 Hz, 2H), 2.80 (q, J=5.1 Hz, 2H), 2.35-1.92 (m, 4H), 1.60 (d, J=6.4 Hz, 3H). LCMS m/z calc 377.35 [M+H]⁺.

### Compound 142

### (4-((4-(((1R,2′S)-7-chloro-2′-methylspiro[isochromane-1,4′-piperidin]-1′-yl)methyl)-1H-1,2,3-triazol-1-yl)methyl)pyridin-2-yl)methanol (142)

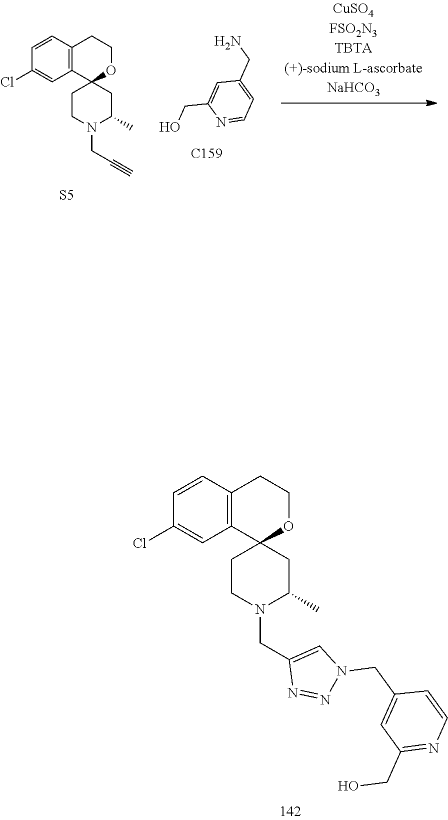

### Synthesis of(4-((4-(((1R,2′S)-7-chloro-2′-methylspiro[isochromane-1,4′-piperidin]-1′-yl)methyl)-1H-1,2,3-triazol-1-yl)methyl)pyridin-2-yl)methanol 142

### Representative Procedure for Reductive Azido Transfer Click Method B

To a solution of (4-(aminomethyl)pyridin-2-yl)methanol (HCl) C159 (14.57 mg, 0.07040 mmol) in DMSO (200 μL) was added NaHCO₃ (110 μL of 1 M, 0.110 mmol). N-diazosulfamoyl fluoride (155 μL of 0.45 M, 0.06975 mmol) as a solution in MTBE was added, and the resulting mixture was stirred at room temperature for 1 hour. A solution of (1R,2′S)-7-chloro-2′-methyl-1′-prop-2-ynyl-spiro[isochromane-1,4′-piperidine]S5 (10 mg, 0.03451 mmol) in DMSO (200 μL) was added, followed by aqueous Copper (II) Sulfate (35 μL of 0.1 M, 0.003500 mmol), aqueous (+)-sodium L-ascorbate (70 μL of 0.2 M, 0.01400 mmol), and a solution of TBTA (35 μL of 0.1 M, 0.003500 mmol) in DMSO. The resulting mixture was heated at 50° C. for 16 h hours. The crude was concentrated and purified by reverse phase HPLC (Method: Waters XSelect CSH C18 OBD Prep Column; 30×150 mm, 5 micron. gradient: acetonitrile in water with 0.2% formic acid) to afford as the formic acid salt (4-((4-(((1R,2′S)-7-chloro-2′-methylspiro[isochromane-1,4′-piperidin]-1′-yl)methyl)-1H-1,2,3-triazol-1-yl)methyl)pyridin-2-yl)methanol 142 (3.5 mg, 22%) LCMS m/z calc 454.24 [M+H]⁺.

Compounds 143-185 were prepared in a single step using the intermediate S5 and azido transfer click method described for compounds 141-142. Amines were obtained from commercial sources. Any modifications to methods are noted in Table 13 and accompanying footnotes.

TABLE 13
Compounds 143-185

| | | Amine | | ¹HNMR; LCMS m/z |
|---|---|---|---|---|
| Cmpd | Product | Reagent | Method | [M + H]⁺ |
| 143 | 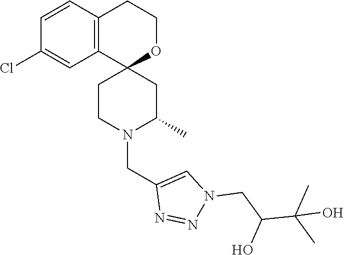 [SMILES: C[C@H]1C[C@@]2(CCN1Cc1cn(CC(O)C(C)(C)O)nn1)OCCc1ccc(Cl)cc12] | 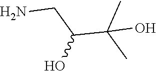 | Cmpd 141 | ¹H NMR (400 MHz, Chloroform-d) δ 7.99 (s, 1H), 7.22 (d, J = 2.0 Hz, 1H), 7.19- 7.02 (m, 2H), 4.79 (dd, J = 14.0, 2.1 Hz, 1H), 4.31 (dd, J = 13.9, 10.0 Hz, 1H), 4.09 (d, 1H), 3.96- 3.79 (m, 3H), 3.78- 3.71 (m, 1H), 2.83- 2.61 (m, 5H), 2.04- 1.83 (m, 3H), 1.75 (t, J = 12.7 Hz, 1H), 1.37- 1.22 (m, 9H). CMS m/z 435.29] [M + H]⁺. |
| 144 | 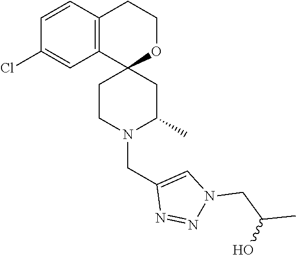 [SMILES: CC(O)Cn1cc(CN2CC[C@]3(C[C@@H]2C)OCCc2ccc(Cl)cc23)nn1] | 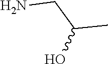 | Cmpd 141 | ¹H NMR (400 MHz, Chloroform-d) δ 7.95 (s, 1H), 7.20 (d, J = 2.0 Hz, 1H), 7.17- 7.01 (m, 2H), 4.44 (dd, J = 13.8, 4.0 Hz, 1H), 4.32 (ddd, J = 13.8, 7.3, 1.6 Hz, 1H), 4.23-4.13 (m, 1H), 4.05 (d, J = 14.5 Hz, 1H), 3.93-3.72 (m, 3H), 2.85-2.56 (m, 5H), 2.03-1.81 (m, 3H), 1.72 (dd, J = 14.0, 11.3 Hz, 1H), 1.26 (d, J = 6.2 Hz, |
| | | | | 3H), 1.19 (dd, J = 6.3, |
| | | | | 1.7 Hz, 3H). LCMS |
| | | | | m/z 391.31 [M + H]⁺. |
| 145 | 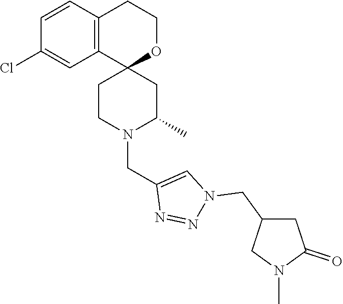 [SMILES: C[C@H]1C[C@@]2(CCN1Cc1cn(CC3CC(=O)N(C)C3)nn1)OCCc1ccc(Cl)cc12] | 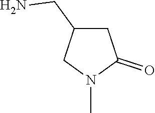 | Cmpd 141 | ¹H NMR (400 MHz, Chloroform-d) δ 8.02 (s, 1H), 7.19 (d, J = 2.0 Hz, 1H), 7.16- 7.01 (m, 2H), 4.52 (d, J = 7.1 Hz, 2H), 4.03 (d, J = 14.5 Hz, 1H), 3.91 (d, J = 14.3 Hz, 1H), 3.86-3.76 (m, 2H), 3.55 (dd, J = 10.3, 8.2 Hz, 1H), 3.34 (d, J = 5.5 Hz, 1H), 3.10-2.92 (m, 1H), 2.86-2.77 (m, 3H), 2.73 (q, J = 5.3 Hz, 3H), 2.64 (td, J = 10.8, 9.4, 5.3 Hz, 1H), 2.55 (dd, J = 17.1, 9.1 Hz, 1H), 2.33-2.22 (m, |
| | | | | 1H), 2.02-1.81 (m, |
| | | | | 4H), 1.72 (dd, J = |
| | | | | 14.1, 11.3 Hz, 1H), |
| | | | | 1.27 (d, 3H). LCMS |
| | | | | m/z 444.32 [M + H]⁺. |
| 146 | 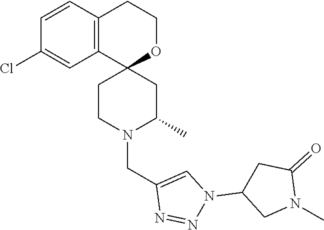 [SMILES: C[C@H]1C[C@@]2(CCN1Cc1cn(C3CC(=O)N(C)C3)nn1)OCCc1ccc(Cl)cc12] | 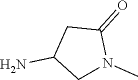 | Cmpd 141 | ¹H NMR (400 MHz, Chloroform-d) δ 8.02 (s, 1H), 7.19 (d, J = 2.0 Hz, 1H), 7.16- 7.01 (m, 2H), 4.52 (d, J = 7.1 Hz, 2H), 4.03 (d, J = 14.5 Hz, 1H), 3.91 (d, J = 14.3 Hz, 1H), 3.86-3.76 (m, 2H), 3.55 (dd, J = 10.3, 8.2 Hz, 1H), 3.34 (d, J = 5.5 Hz, 1H), 3.10-2.92 (m, 1H), 2.86-2.77 (m, 3H), |
| | | | | 2.73 (q, J = 5.3 Hz, |
| | | | | 3H), 2.64 (td, J = 10.8, |
| | | | | 9.4, 5.3 Hz, 1H), 2.55 |
| | | | | (dd, J = 17.1, 9.1 Hz, |
| | | | | 1H), 2.33-2.22 (m, |
| | | | | 1H), 2.02-1.81 (m, |
| | | | | 4H), 1.72 (dd, J = |
| | | | | 14.1, 11.3 Hz, 1H), |
| | | | | 1.27 (d, 3H). LCMS |
| | | | | m/z 430.27 [M + H]⁺. |
| 147 | 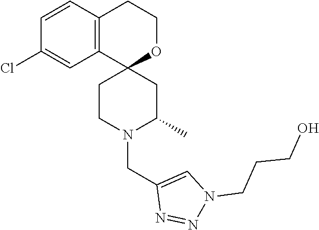 [SMILES: C[C@H]1C[C@@]2(CCN1Cc1cn(CCCO)nn1)OCCc1ccc(Cl)cc12] | 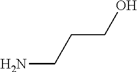 | Cmpd 141 | ¹H NMR (400 MHz, Chloroform-d) δ 8.29 (s, 1H), 7.29-7.12 (m, 3H), 4.73 (d, J = 14.4 Hz, 1H), 4.69-4.52 (m, 3H), 3.90 (hept, J = 5.6 Hz, 2H), 3.61 (t, J = 6.0 Hz, 3H), 3.47 (q, J = 6.9, 5.3 Hz, 2H), 2.90-2.71 (m, 2H), 2.30-2.00 (m, 6H), 1.61 (d, J = 6.4 Hz, 3H). LCMS m/z 391.31 [M + H]⁺. |
| 148 | 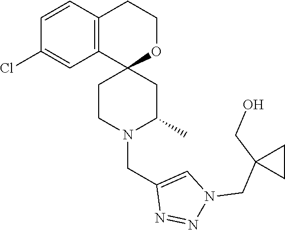 [SMILES: C[C@H]1C[C@@]2(CCN1Cc1cn(CC3(CO)CC3)nn1)OCCc1ccc(Cl)cc12] | 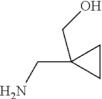 | Cmpd 141 | ¹H NMR (400 MHz, Chloroform-d) δ 8.30 (s, 1H), 7.29-6.99 (m, 3H), 4.72 (d, J = 14.4 Hz, 1H), 4.58 (d, J = 14.4 Hz, 1H), 4.50 (d, J = 2.2 Hz, 2H), 3.87 (hept, J = 5.7 Hz, 2H), 3.59 (s, 2H), 3.45 (q, J = 5.2 Hz, 2H), 2.79 (q, J = 5.0 Hz, 2H), 2.27- 2.01 (m, 5H), 1.59 (d, J = 6.4 Hz, 3H), 0.82- 0.73 (m, 2H), 0.68- |
| | | | | 0.59 (m, 2H). LCMS |
| | | | | m/z 417.31 [M + H]⁺. |
| 149 | 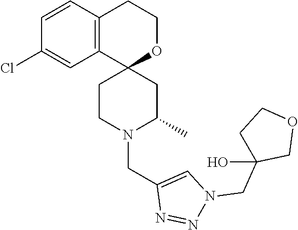 [SMILES: C[C@H]1C[C@@]2(CCN1Cc1cn(CC3(O)CCOC3)nn1)OCCc1ccc(Cl)cc12] | 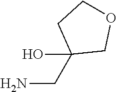 | Cmpd 141 | ¹H NMR (400 MHz, Chloroform-d) δ 7.97 (s, 1H), 7.19 (s, 1H), 7.14-7.01 (m, 2H), 4.60 (s, 2H), 4.10- 3.86 (m, 4H), 3.85- 3.74 (m, 3H), 3.56 (dd, J = 9.4, 3.4 Hz, 1H), 2.81-2.70 (m, 3H), 2.70-2.59 (m, 2H), 2.17-2.06 (m, 1H), 2.00-1.80 (m, 4H), 1.72 (dd, J = 14.1, 11.3 Hz, 1H), |
| | | | | 1.26 (d, J = 6.2 Hz, |
| | | | | 3H). LCMS m/z |
| | | | | 433.30 [M + H]⁺. |
| 150 | 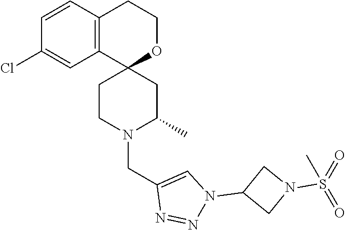 [SMILES: C[C@H]1C[C@@]2(CCN1Cc1cn(C3CN(S(C)(=O)=O)C3)nn1)OCCc1ccc(Cl)cc12] | 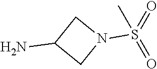 | Cmpd 141 | ¹H NMR (400 MHz, Chloroform-d) δ 8.12 (s, 1H), 7.25-7.17 (m, 1H), 7.15-6.96 (m, 2H), 5.61-5.42 (m, 1H), 4.54-4.36 (m, 4H), 4.06 (d, J = 14.6 Hz, 1H), 3.90 (d, J = 14.5 Hz, 1H), 3.85- 3.74 (m, 2H), 3.08 (s, 3H), 2.83-2.54 (m, 5H), 2.01-1.81 (m, 3H), 1.72 (dd, J = 13.9, 11.3 Hz, 1H), |
| | | | | 1.28 (d, 3H). LCMS |
| | | | | m/z 466.31 [M + H]⁺. |
| 151 | 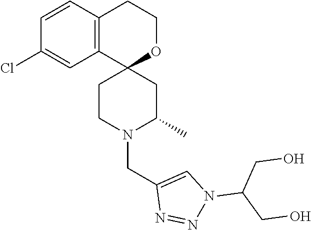 | 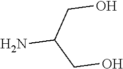 | Cmpd 141 | ¹H NMR (400 MHz, Chloroform-d) δ 8.02 (s, 1H), 7.20 (d, J = 2.0 Hz, 1H), 7.15- 7.00 (m, 2H), 4.75- 4.64 (m, 1H), 4.08 (d, J = 14.3 Hz, 1H), 4.04- 3.94 (m, 4H), 3.85- 3.75 (m, 3H), 2.84- 2.59 (m, 5H), 2.02- 1.81 (m, 3H), 1.73 (dd, J = 14.0, 11.3 Hz, 1H), 1.35-1.22 (m, 3H). LCMS m/z |
| | | | | 407.33 [M + H]⁺. |
| 152 | 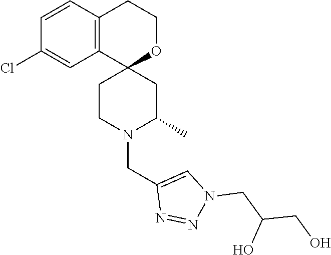 [SMILES: C[C@H]1C[C@@]2(CCN1Cc1cn(CC(O)CO)nn1)OCCc1ccc(Cl)cc12] | 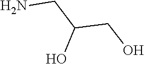 | Cmpd 141 | ¹H NMR (400 MHz, Chloroform-d) δ 7.97 (s, 1H), 7.20 (d, J = 2.0 Hz, 1H), 7.16- 7.01 (m, 2H), 4.61 (dd, J = 14.0, 3.7 Hz, 1H), 4.40 (ddd, J = 14.0, 7.8, 1.7 Hz, 1H), 4.10-3.98 (m, 2H), 3.91-3.77 (m, 3H), 3.63-3.45 (m, 2H), 2.84-2.51 (m, 5H), 2.03-1.79 (m, 3H), 1.72 (dd, J = 14.0, 11.3 Hz, 1H), 1.27 (d, J = 6.3 Hz, 3H). |
| | | | | LCMS m/z 407.33 |
| | | | | [M + H]⁺. |
| 153 | 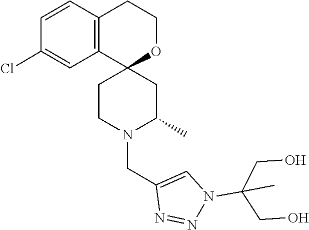 [SMILES: C[C@H]1C[C@@]2(CCN1Cc1cn(C(C)(CO)CO)nn1)OCCc1ccc(Cl)cc12] | 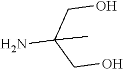 | Cmpd 141 | ¹H NMR (400 MHz, Chloroform-d) δ 8.38 (s, 1H), 7.31-6.98 (m, 3H), 4.76 (d, J = 14.3 Hz, 1H), 4.53 (d, J = 14.3 Hz, 1H), 4.04 (dd, J = 11.6, 2.7 Hz, 2H), 3.89 (d, J = 11.6 Hz, 4H), 3.67 (s, 1H), 3.48 (t, J = 5.8 Hz, 2H), 2.81 (q, J = 5.0 Hz, 2H), 2.34-1.99 (m, 4H), 1.71 (s, 3H), 1.60 (d, J = 6.4 Hz, |
| | | | | 3H). LCMS m/z |
| | | | | 421.38 [M + H]⁺. |
| 154 | 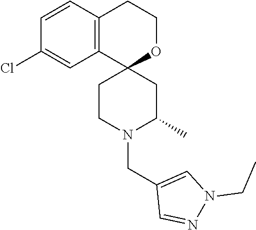 [SMILES: CCn1cc(CN2CC[C@]3(C[C@@H]2C)OCCc2ccc(Cl)cc23)cn1] | 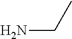 | Cmpd 141 | ¹H NMR (400 MHz, DMSO-d6) δ 8.38 (s, 1H), 7.32-7.24 (m, 1H), 7.19 (d, J = 8.2 Hz, 1H), 7.14 (d, J = 2.9 Hz, 1H), 4.65 (d, J = 14.4 Hz, 1H), 4.57- 4.40 (m, 3H), 3.82 (dd, J = 16.0, 10.0 Hz, 2H), 3.09 (qd, J = 7.3, 4.5 Hz, 3H), 2.72 (s, 2H), 2.11 (hept, J = 13.9, 13.3 Hz, 4H), 1.53-1.41 (m, 6H). |
| | | | | LCMS m/z 361.37 |
| | | | | [M + H]⁺. |
| 155 | 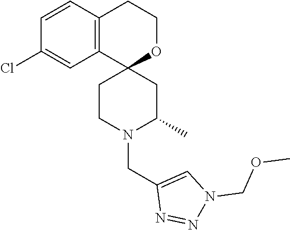 [SMILES: COCn1cc(CN2CC[C@]3(C[C@@H]2C)OCCc2ccc(Cl)cc23)nn1] | 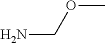 | Cmpd 141 | ¹H NMR (400 MHz, DMSO) δ 8.15 (s, 1H), 7.38-7.08 (m, 3H), 5.72 (s, 2H), 4.72 (s, 1H), 4.60 (s, 1H), 3.79 (s, 2H), 3.32 (s, 2H), 3.26-3.01 (m, 4H), 2.72 (s, 2H), 2.11 (d, J = 22.0 Hz, 4H), 1.44 (d, J = 6.3 Hz, 3H). LCMS m/z 377.35 [M + H]⁺. |
| 156 | 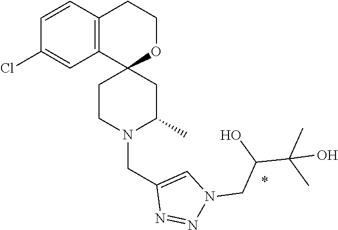 [SMILES: C[C@H]1C[C@@]2(CCN1Cc1cn(CC(O)C(C)(C)O)nn1)OCCc1ccc(Cl)cc12] | 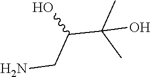 | Cmpd 141¹ | ¹H NMR (300 MHz, Chloroform-d) δ 7.96 (s, 1H), 7.19 (d, J = 2.1 Hz, 1H), 7.17- 6.99 (m, 2H), 4.77 (dd, J = 13.9, 2.3 Hz, 1H), 4.29 (dd, J = 13.9, 10.0 Hz, 1H), 4.05 (d, J = 14.5 Hz, 1H), 3.94-3.79 (m, 3H), 3.71 (dd, J = 10.0, 2.2 Hz, 1H), 2.84- 2.55 (m, 5H), 2.01- 1.64 (m, 4H), 1.26 |
| | | | | (dd, J = 7.4, 3.6 Hz, |
| | | | | 9H). LCMS m/z |
| | | | | 435.39 [M + H]⁺. |
| 157 | 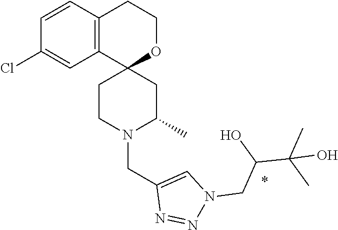 [SMILES: C[C@H]1C[C@@]2(CCN1Cc1cn(CC(O)C(C)(C)O)nn1)OCCc1ccc(Cl)cc12] | 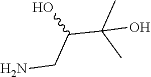 | Cmpd 141¹ | ¹H NMR (300 MHz, Chloroform-d) δ 7.96 (s, 1H), 7.20 (d, J = 2.1 Hz, 1H), 7.17- 6.99 (m, 2H), 4.76 (dd, J = 13.8, 2.2 Hz, 1H), 4.29 (dd, J = 13.9, 10.0 Hz, 1H), 4.05 (d, J = 14.5 Hz, 1H), 3.94-3.77 (m, 3H), 3.72 (dd, J = 10.0, 2.3 Hz, 1H), 2.84- 2.56 (m, 5H), 2.02- 1.65 (m, 4H), 1.36- |
| | | | | 1.13 (m, 9H). |
| | | | | LCMS m/z 435.39 |
| | | | | [M + H]⁺. |
| 158 | 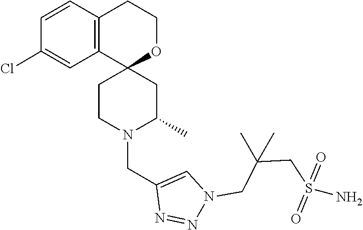 [SMILES: C[C@H]1C[C@@]2(CCN1Cc1cn(CC(C)(C)CS(N)(=O)=O)nn1)OCCc1ccc(Cl)cc12] | 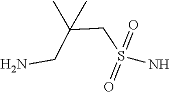 | Cmpd 142 | ¹H NMR (400 MHz, DMSO-d6) δ 11.23 (s, 1H), 8.41 (s, 1H), 7.33- 7.14 (m, 2H), 7.12- 6.97 (m, 1H), 4.59- 4.39 (m, 3H), 3.92- 3.70 (m, 2H), 3.35- 3.23 (m, 2H), 3.13 (s, 2H), 2.82-2.67 (m, 3H), 2.54 (s, 2H), 2.44- 2.29 (m, 2H), 2.21- 1.92 (m, 2H), 1.50 (d, J = 6.3 Hz, 3H), 1.11 (s, 3H), 1.07 (s, 3H). |
| | | | | LCMS m/z 482.33 |
| | | | | [M + H]⁺. |
| 159 | 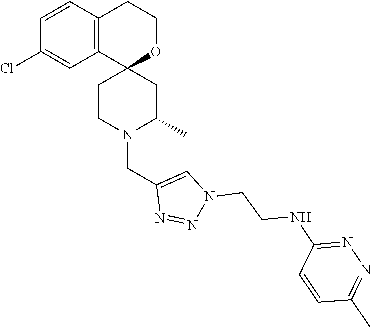 [SMILES: Cc1ccc(NCCn2cc(CN3CC[C@]4(C[C@@H]3C)OCCc3ccc(Cl)cc34)nn2)nn1] | 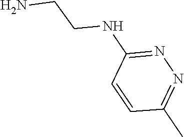 | Cmpd 142 | ¹H NMR (400 MHz, DMSO-d6) δ 8.47 (s, 1H), 7.78 (d, J = 9.3 Hz, 1H), 7.55 (d, J = 9.4 Hz, 1H), 7.30- 7.16 (m, 3H), 4.71 (t, J = 5.7 Hz, 2H), 4.63- 4.39 (m, 2H), 3.94- 3.70 (m, 4H), 3.26- 2.99 (m, 2H), 2.79- 2.61 (m, 3H), 2.51 (s, 4H), 2.44-2.26 (m, 2H), 2.15-1.94 (m, 2H), 1.49 (d, J = 6.3 Hz, 3H). LCMS m/z 468.38 [M + H]⁺. |
| 160 | 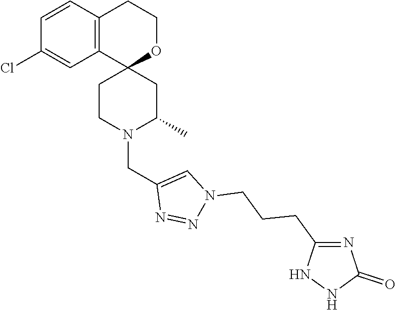 [SMILES: C[C@H]1C[C@@]2(CCN1Cc1cn(CCCc3nc(=O)[nH][nH]3)nn1)OCCc1ccc(Cl)cc12] | 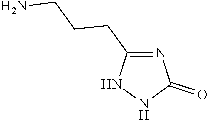 | Cmpd 142 | ¹H NMR (400 MHz, DMSO-d6) δ 11.33- 11.11 (m, 2H), 8.40 (s, 1H), 7.36-7.11 (m, 3H), 4.51 (dt, J = 14.4, 7.0 Hz, 4H), 3.92- 3.64 (m, 2H), 3.11 (t, 2H), 2.88-2.66 (m, 3H), 2.58-2.53 (m, 2H), 2.46-2.29 (m, 2H), 2.24-1.92 (m, 4H), 1.50 (d, J = 6.3 Hz, 3H). LCMS m/z 458.36 [M + H]⁺. |
| 161 | 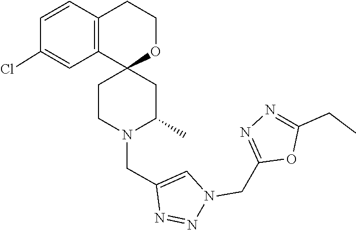 [SMILES: CCc1nnc(Cn2cc(CN3CC[C@]4(C[C@@H]3C)OCCc3ccc(Cl)cc34)nn2)o1] | 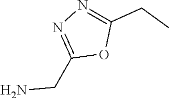 | Cmpd 142 | ¹H NMR (400 MHz, DMSO-d6) δ 8.39 (d, J = 4.3 Hz, 1H), 7.40- 7.03 (m, 3H), 5.39 (s, 2H), 4.58 (s, 2H), 3.94- 3.68 (m, 2H), 3.24- 3.00 (m, 5H), 2.83- 2.63 (m, 2H), 2.40 (dd, J = 31.4, 16.7 Hz, 3H), 2.24-1.97 (m, 4H), 1.51 (d, J = 6.3 Hz, 3H). LCMS m/z 443.24 [M + H]⁺. |
| 162 | 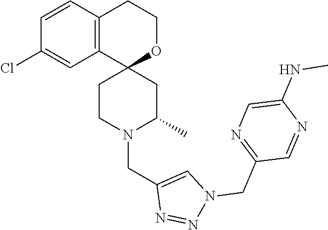 [SMILES: CNc1cnc(Cn2cc(CN3CC[C@]4(C[C@@H]3C)OCCc3ccc(Cl)cc34)nn2)cn1] | 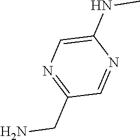 | Cmpd 142 | ¹H NMR (400 MHz, DMSO-d6) δ 8.06 (s, 1H), 7.98 (s, 1H), 7.87 (s, 1H), 7.25 (s, 1H), 7.22-7.16 (m, 2H), 7.15-7.07 (m, 1H), 5.48 (s, 2H), 3.92- 3.79 (m, 1H), 3.76- 3.64 (m, 3H), 2.79 (d, J = 4.7 Hz, 3H), 2.72- 2.53 (m, 5H), 1.85 (dd, J = 12.9, 4.5 Hz, 1H), 1.74 (d, J = 13.1 Hz, 2H), 1.62 (d, J = 12.7 Hz, 1H), 1.14 (d, |
| | | | | J = 6.1 Hz, 3H). |
| | | | | LCMS m/z 454.29 |
| | | | | [M + H]⁺. |
| 163 | 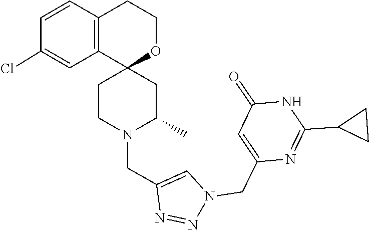 [SMILES: C[C@H]1C[C@@]2(CCN1Cc1cn(Cc3cc(=O)[nH]c(C4CC4)n3)nn1)OCCc1ccc(Cl)cc12] | 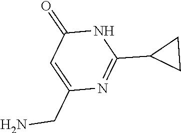 [SMILES: NCc1cc(=O)[nH]c(C2CC2)n1] | Cmpd 142 | ¹H NMR (400 MHz, DMSO-d6) δ 8.24 (s, 1H), 7.33-7.05 (m, 3H), 5.72 (s, 1H), 5.40 (s, 2H), 4.46-4.04 (m, 2H), 3.79 (p, J = 6.1 Hz, 2H), 3.06-2.75 (m, 1H), 2.70 (t, J = 5.2 Hz, 3H), 2.55 (s, 2H), 2.18-2.01 (m, 1H), 2.00-1.79 (m, 3H), 1.33 (d, J = 5.7 Hz, 3H), 1.10-0.97 (m, 2H), 0.95-0.83 |
| | | | | (m, 2H). LCMS m/z |
| | | | | 481.30 [M + H]⁺. |
| 164 | 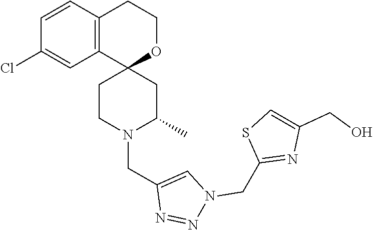 [SMILES: C[C@H]1C[C@@]2(CCN1Cc1cn(Cc3nc(CO)cs3)nn1)OCCc1ccc(Cl)cc12] | 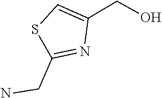 | Cmpd 142 | ¹H NMR (400 MHz, DMSO-d6) δ 8.18 (s, 1H), 8.15 (s, 1H), 7.44 (s, 1H), 7.26 (s, 1H), 7.21-7.05 (m, 2H), 5.96 (s, 2H), 4.54 (s, 2H), 3.89 (d, 1H), 3.85- 3.66 (m, 3H), 2.74- 2.61 (m, 3H), 2.57- 2.53 (m, 2H), 1.95- 1.82 (m, 1H), 1.82- 1.72 (m, 2H), 1.68- 1.62 (m, 1H), 1.16 (d, J = 6.1 Hz, 3H). |
| | | | | LCMS m/z 460.26 |
| | | | | [M + H]⁺. |
| 165 | 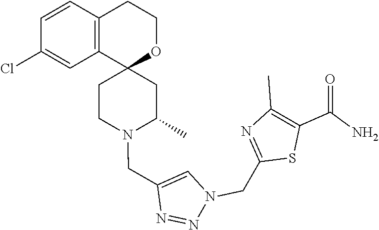 [SMILES: Cc1nc(Cn2cc(CN3CC[C@]4(C[C@@H]3C)OCCc3ccc(Cl)cc34)nn2)sc1C(N)=O] | 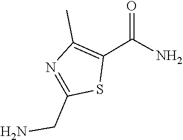 | Cmpd 142 | ¹H NMR (400 MHz, DMSO-d6) δ 8.26 (s, 1H), 8.16 (s, 1H), 7.75- 7.45 (m, 2H), 7.26 (s, 1H), 7.22-7.04 (m, 2H), 5.96 (s, 2H), 3.90 (d, J = 14.6 Hz, 1H), 3.74 (td, J = 13.9, 12.0, 8.3 Hz, 3H), 2.75- 2.57 (m, 5H), 2.54 (s, 3H), 1.93-1.80 (m, 1H), 1.79-1.71 (m, 2H), 1.70-1.56 (m, 1H), 1.15 (d, J = 6.1 |
| | | | | Hz, 3H). LCMS m/z |
| | | | | 487.26 [M + H]⁺. |
| 166 | 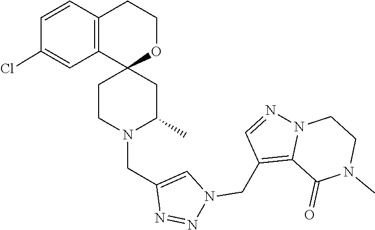 [SMILES: C[C@H]1C[C@@]2(CCN1Cc1cn(Cc3cnn4c3C(=O)N(C)CC4)nn1)OCCc1ccc(Cl)cc12] | 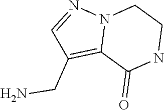 | Cmpd 142 | ¹H NMR (400 MHz, DMSO-d6) δ 8.18 (s, 1H), 8.00 (s, 1H), 7.50 (s, 1H), 7.30-7.24 (m, 1H), 7.21-7.05 (m, 2H), 5.70 (s, 2H), 4.38 (t, J = 6.2 Hz, 2H), 3.91-3.61 (m, 6H), 3.03 (s, 3H), 2.75- 2.56 (m, 5H), 1.85 (dt, J = 13.1, 6.5 Hz, 1H), 1.75 (d, J = 13.0 Hz, 2H), 1.63 (t, J = 12.3 Hz, 1H), 1.14 (d, J = |
| | | | | 6.1 Hz, 3H). LCMS |
| | | | | m/z 496.29 [M + H]⁺. |
| 167 | 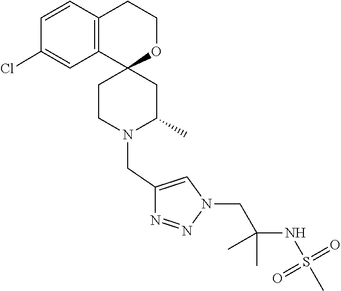 [SMILES: C[C@H]1C[C@@]2(CCN1Cc1cn(CC(C)(C)NS(C)(=O)=O)nn1)OCCc1ccc(Cl)cc12] | 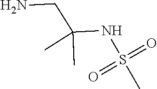 | Cmpd 142 | ¹H NMR (400 MHz, DMSO-d6) δ 8.16 (s, 1H), 7.94 (s, 1H), 7.25 (s, 1H), 7.21-7.06 (m, 2H), 4.48 (s, 2H), 4.02- 3.90 (m, 1H), 3.87- 3.66 (m, 3H), 2.97 (s, 3H), 2.75-2.61 (m, 3H), 2.60-2.54 (m, 2H), 1.99-1.82 (m, 1H), 1.80-1.73 (m, 2H), 1.73-1.59 (m, 1H), 1.25 (s, 3H), 1.24 (s, 3H), 1.18 (d, J = 6.2 Hz, 3H). LCMS m/z 482.29 [M + H]⁺. |
| 168 | 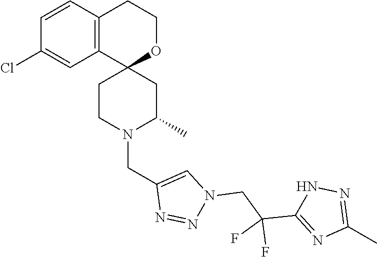 [SMILES: Cc1n[nH]c(C(F)(F)Cn2cc(CN3CC[C@]4(C[C@@H]3C)OCCc3ccc(Cl)cc34)nn2)n1] | 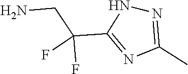 | Cmpd 142 | ¹H NMR (400 MHz, DMSO-d6) δ 8.20 (s, 1H), 8.02 (s, 1H), 7.26 (s, 1H), 7.22-7.05 (m, 2H), 5.36 (t, J = 14.7 Hz, 2H), 3.92 (d, J = 14.5 Hz, 1H), 3.84- 3.62 (m, 3H), 2.75- 2.55 (m, 5H), 2.43 (s, 3H), 1.93-1.80 (m, 1H), 1.78-1.69 (m, 2H), 1.67-1.54 (m, 1H), 1.15 (d, J = 6.1 Hz, 3H). LCMS m/z 478.27 [M + H]⁺. |
| 169 | 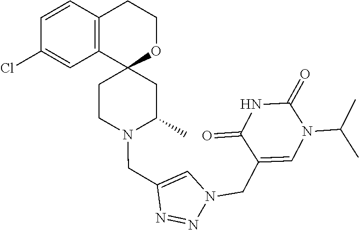 [SMILES: CC(C)n1cc(Cn2cc(CN3CC[C@]4(C[C@@H]3C)OCCc3ccc(Cl)cc34)nn2)c(=O)[nH]c1=O] | 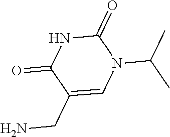 | Cmpd 142 | ¹H NMR (400 MHz, DMSO-d6) δ 11.49 (s, 1H), 8.14 (s, 1H), 8.06 (s, 1H), 7.30-7.19 (m, 2H), 7.20-7.08 (m, 1H), 5.22 (s, 2H), 4.67 (q, J = 6.9 Hz, 1H), 4.20-4.05 (m, 1H), 4.03-3.88 (m, 1H), 3.81-3.62 (m, 2H), 2.91-2.63 (m, 5H), 2.01-1.69 (m, 4H), 1.28 (d, J = 6.8 Hz, 6H), 1.23 (d, J = 6.8 |
| | | | | Hz, 4H). LCMS m/z |
| | | | | 499.31 [M + H]⁺. |
| 170 | 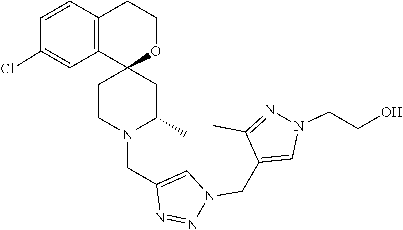 [SMILES: Cc1nn(CCO)cc1Cn1cc(CN2CC[C@]3(C[C@@H]2C)OCCc2ccc(Cl)cc23)nn1] | 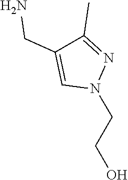 | Cmpd 142 | ¹H NMR (400 MHz, DMSO-d6) δ 8.21 (s, 1H), 7.90 (s, 1H), 7.70 (s, 1H), 7.24 (s, 1H), 7.22-7.08 (m, 2H), 5.38 (s, 2H), 4.09- 3.98 (m, 2H), 3.85 (d, J = 14.5 Hz, 1H), 3.77- 3.64 (m, 5H), 2.74- 2.65 (m, 2H), 2.63- 2.43 (m, 3H), 2.10 (s, 3H), 1.90-1.80 (m, 1H), 1.78-1.71 (m, 2H), 1.69-1.54 (m, 1H), 1.14 (d, J = 6.1 |
| | | | | Hz, 3H). LCMS m/z |
| | | | | 471.32 [M + H]⁺. |
| 171 | 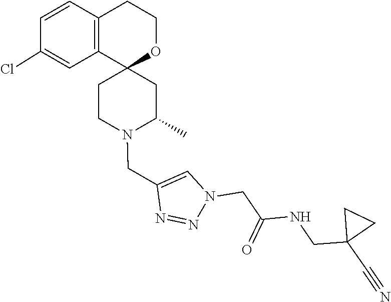 [SMILES: C[C@H]1C[C@@]2(CCN1Cc1cn(CC(=O)NCC3(C#N)CC3)nn1)OCCc1ccc(Cl)cc12] | 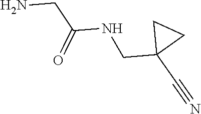 [SMILES: N#CC1(CNC(=O)CN)CC1] | Cmpd 142 | ¹H NMR (400 MHz, DMSO-d6) δ 8.83 (t, J = 6.0 Hz, 1H), 8.20 (s, 1H), 7.96 (s, 1H), 7.26 (s, 1H), 7.23-7.07 (m, 2H), 5.16 (s, 2H), 3.91 (d, J = 14.6 Hz, 1H), 3.74 (td, J = 11.8, 5.1 Hz, 3H), 3.29 (d, J = 5.8 Hz, 2H), 2.77- 2.56 (m, 5H), 1.97- 1.83 (m, 1H), 1.80- 1.71 (m, 2H), 1.70- 1.55 (m, 1H), 1.29- 1.20 (m, 2H), 1.16 (d, J = 6.1 Hz, 3H), 1.12- 1.00 (m, 2H). LCMS m/z 469.29 [M + H]⁺. |
| 172 | 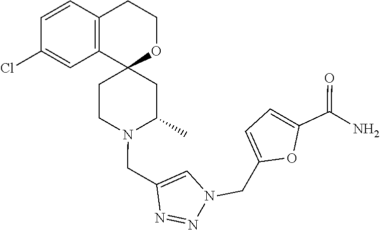 [SMILES: C[C@H]1C[C@@]2(CCN1Cc1cn(Cc3ccc(C(N)=O)o3)nn1)OCCc1ccc(Cl)cc12] | 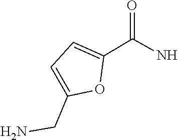 | Cmpd 142 | ¹H NMR (400 MHz, DMSO-d6) δ 8.22 (s, 1H), 8.10 (s, 1H), 7.81 (s, 1H), 7.43 (s, 1H), 7.25 (d, J = 2.2 Hz, 1H), 7.22-7.05 (m, 3H), 6.62 (d, J = 3.4 Hz, 1H), 5.69 (s, 2H), 3.97-3.85 (m, 1H), 3.73 (dt, J = 9.4, 4.5 Hz, 3H), 2.73-2.54 (m, 5H), 1.94-1.83 (m, 1H), 1.75 (d, J = 13.3 Hz, 2H), 1.68- |
| | | | | 1.53 (m, 1H), 1.15 (d, |
| | | | | J = 6.1 Hz, 3H). |
| | | | | LCMS m/z 456.28 |
| | | | | [M + H]⁺. |
| 173 | 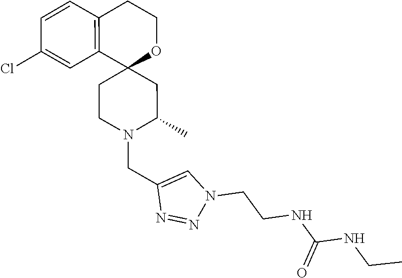 [SMILES: CCNC(=O)NCCn1cc(CN2CC[C@]3(C[C@@H]2C)OCCc2ccc(Cl)cc23)nn1] | 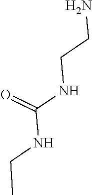 | Cmpd 142 | ¹H NMR (400 MHz, DMSO-d6) δ 8.25 (s, 1H), 7.95 (s, 1H), 7.25 (s, 1H), 7.22-7.04 (m, 2H), 6.04-5.92 (m, 2H), 4.37 (t, J = 6.2 Hz, 2H), 3.89 (d, J = 14.5 Hz, 1H), 3.82- 3.61 (m, 3H), 3.58- 3.47 (m, 2H), 3.07- 2.94 (m, 2H), 2.75- 2.58 (m, 5H), 1.92- 1.80 (m, 1H), 1.80- 1.67 (m, 2H), 1.69- 1.56 (m, 1H), 1.15 (d, J = 6.1 Hz, 3H), 0.96 (t, J = 7.2 Hz, 3H). |
| | | | | LCMS m/z 447.30 |
| | | | | [M + H]⁺. |
| 174 | 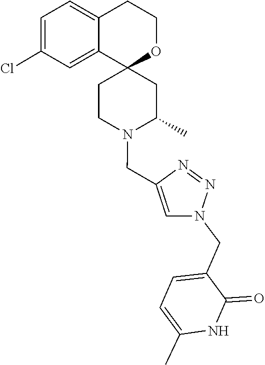 [SMILES: Cc1ccc(Cn2cc(CN3CC[C@]4(C[C@@H]3C)OCCc3ccc(Cl)cc34)nn2)c(=O)[nH]1] | 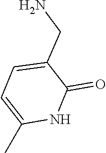 | Cmpd 142 | ¹H NMR (400 MHz, DMSO-d6) δ 8.20 (s, 1H), 7.96 (s, 1H), 7.29- 7.22 (m, 2H), 7.21- 7.06 (m, 2H), 6.01 (d, J = 7.0 Hz, 1H), 5.29 (s, 2H), 3.88 (d, J = 14.5 Hz, 1H), 3.79- 3.58 (m, 3H), 2.75- 2.54 (m, 5H), 2.17 (s, 3H), 1.92-1.82 (m, 1H), 1.78-1.70 (m, 2H), 1.67-1.56 (m, 1H), 1.14 (d, J = 6.1 Hz, 3H). LCMS m/z 454.29 [M + H]⁺. |
| 175 | 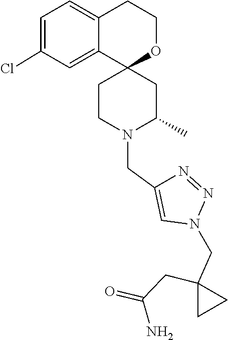 [SMILES: C[C@H]1C[C@@]2(CCN1Cc1cn(CC3(CC(N)=O)CC3)nn1)OCCc1ccc(Cl)cc12] | 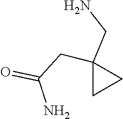 | Cmpd 141 | ¹H NMR (400 MHz, DMSO-d6) δ 8.18 (s, 1H), 8.00 (s, 1H), 7.32- 7.22 (m, 2H), 7.21- 7.07 (m, 2H), 6.87 (s, 1H), 4.35 (s, 2H), 3.91 (d, J = 14.5 Hz, 1H), 3.86-3.68 (m, 3H), 2.77-2.52 (m, 5H), 1.93 (s, 2H), 1.91- 1.84 (m, 1H), 1.81- 1.72 (m, 2H), 1.70- 1.56 (m, 1H), 1.16 (d, J = 6.1 Hz, 3H), 0.76- 0.67 (m, 2H), 0.58- 0.46 (m, 2H). LCMS m/z 444.27 [M + H]⁺. |
| 176 | 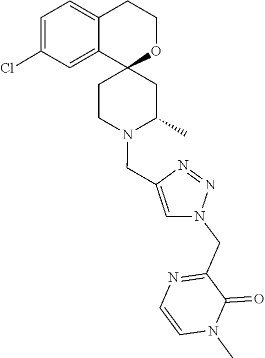 [SMILES: C[C@H]1C[C@@]2(CCN1Cc1cn(Cc3nccn(C)c3=O)nn1)OCCc1ccc(Cl)cc12] | 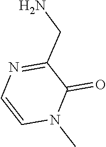 | Cmpd 142 | ¹H NMR (400 MHz, DMSO-d6) δ 8.22 (s, 1H), 7.99 (s, 1H), 7.71 (d, J = 4.3 Hz, 1H), 7.32-7.23 (m, 2H), 7.22-7.07 (m, 2H), 5.61 (s, 2H), 3.91 (d, J = 14.5 Hz, 1H), 3.82- 3.64 (m, 3H), 3.48 (s, 3H), 2.77-2.52 (m, 5H), 1.95-1.81 (m, 1H), 1.79-1.71 (m, 2H), 1.68-1.56 (m, 1H), 1.15 (d, J = 6.1 Hz, 3H). LCMS m/z 455.25 [M + H]⁺. |
| 177 | 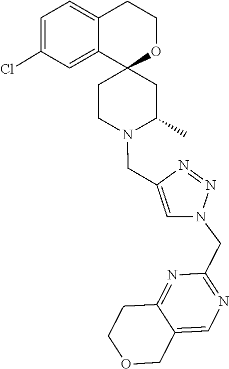 [SMILES: C[C@H]1C[C@@]2(CCN1Cc1cn(Cc3ncc4c(n3)CCOC4)nn1)OCCc1ccc(Cl)cc12] | 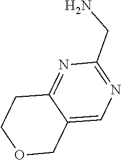 [SMILES: NCc1ncc2c(n1)CCOC2] | Cmpd 142 | ¹H NMR (400 MHz, DMSO-d6) δ 8.49 (s, 1H), 8.06 (s, 1H), 7.26 (d, J = 2.2 Hz, 1H), 7.24-7.05 (m, 2H), 5.77 (s, 2H), 4.72 (s, 2H), 4.02-3.85 (m, 3H), 3.80-3.65 (m, 3H), 2.84 (t, J = 5.8 Hz, 2H), 2.54 (s, 5H), 1.95-1.82 (m, 1H), 1.82-1.72 (m, 2H), 1.69-1.55 (m, 1H), 1.16 (d, J = 6.1 Hz, 3H). LCMS m/z 481.30 [M + H]⁺. |
| 178 | 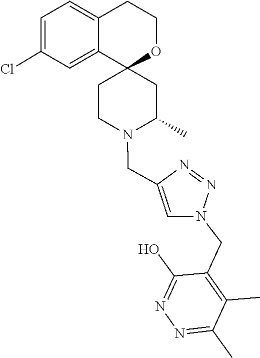 [SMILES: Cc1nnc(O)c(Cn2cc(CN3CC[C@]4(C[C@@H]3C)OCCc3ccc(Cl)cc34)nn2)c1C] | 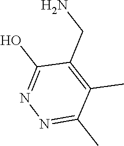 | Cmpd 142 | LCMS m/z 469.24 [M + H]⁺. |
| 179 | 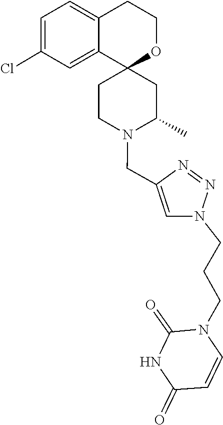 [SMILES: C[C@H]1C[C@@]2(CCN1Cc1cn(CCCn3ccc(=O)[nH]c3=O)nn1)OCCc1ccc(Cl)cc12] | 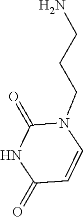 [SMILES: NCCCn1ccc(=O)[nH]c1=O] | Cmpd 142 | ¹H NMR (400 MHz, DMSO-d6) δ 11.26 (s, 1H), 8.06 (s, 1H), 7.60 (d, J = 7.8 Hz, 1H), 7.24 (s, 1H), 7.23- 7.05 (m, 2H), 5.56 (d, J = 7.9 Hz, 1H), 4.40 (t, J = 7.1 Hz, 2H), 3.92 (d, J = 14.5 Hz, 1H), 3.73 (dq, J = 20.4, 6.9, 6.3 Hz, 5H), 2.66 (dd, J = 12.9, 8.4 Hz, 3H), 2.58-2.53 (m, 2H), 2.17 (t, J = 7.0 Hz, 2H), 1.94- 1.84 (m, 1H), 1.83- 1.74 (m, 2H), 1.71- 1.61 (m, 1H), 1.17 (d, J = 6.1 Hz, 3H). LCMS m/z 485.31 [M + H]⁺. |
| 180 | 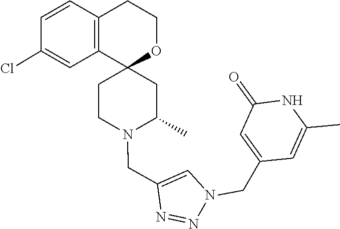 [SMILES: Cc1cc(Cn2cc(CN3CC[C@]4(C[C@@H]3C)OCCc3ccc(Cl)cc34)nn2)cc(=O)[nH]1] | 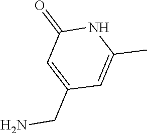 | Cmpd 142 | ¹H NMR (400 MHz, DMSO-d6) δ 11.60 (s, 1H), 8.11 (s, 1H), 7.25 (s, 1H), 7.22-7.04 (m, 2H), 5.79 (s, 2H), 5.42 (s, 2H), 4.06-3.69 (m, 4H), 2.85-2.61 (m, 3H), 2.59-2.54 (m, 2H), 2.13 (s, 3H), 1.96- 1.85 (m, 1H), 1.85- 1.75 (m, 2H), 1.74- 1.59 (m, 1H), 1.17 (d, J = 6.2 Hz, 3H). LCMS m/z 454.25 |
| | | | | [M + H]⁺. |
| 181 | 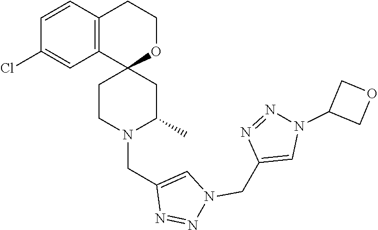 [SMILES: C[C@H]1C[C@@]2(CCN1Cc1cn(Cc3cn(C4COC4)nn3)nn1)OCCc1ccc(Cl)cc12] | 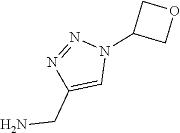 [SMILES: NCc1cn(C2COC2)nn1] | Cmpd 142 | ¹H NMR (400 MHz, DMSO-d6) δ 8.43 (s, 1H), 8.09 (s, 1H), 7.31- 7.17 (m, 2H), 7.16- 7.06 (m, 1H), 5.85 (t, J = 6.8 Hz, 1H), 5.72 (s, 2H), 5.00 (t, J = 7.3 Hz, 2H), 4.89 (t, J = 6.5 Hz, 2H), 4.03- 3.88 (m, 1H), 3.83- 3.61 (m, 3H), 2.77- 2.61 (m, 3H), 2.58- 2.54 (m, 2H), 1.95- 1.59 (m, 4H), 1.18 (d, |
| | | | | J = 6.1 Hz, 3H). |
| | | | | LCMS m/z 470.28 |
| | | | | [M + H]⁺. |
| 182 | 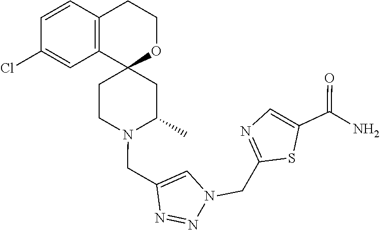 [SMILES: C[C@H]1C[C@@]2(CCN1Cc1cn(Cc3ncc(C(N)=O)s3)nn1)OCCc1ccc(Cl)cc12] | 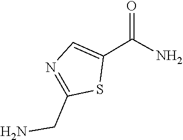 | Cmpd 141 | ¹H NMR (400 MHz, DMSO-d6) δ 8.31 (s, 1H), 8.22-8.04 (m, 2H), 7.68 (s, 1H), 7.25 (s, 1H), 7.21-7.09 (m, 2H), 6.02 (s, 2H), 3.99- 3.67 (m, 4H), 2.74- 2.62 (m, 3H), 2.62- 2.53 (m, 2H), 1.95- 1.83 (m, 1H), 1.83- 1.73 (m, 2H), 1.73- 1.58 (m, 1H), 1.17 (d, J = 6.1 Hz, 3H). LCMS m/z 473.26 |
| | | | | [M + H]⁺. |
| 183 | 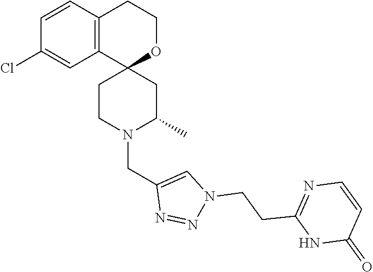 [SMILES: C[C@H]1C[C@@]2(CCN1Cc1cn(CCc3nccc(=O)[nH]3)nn1)OCCc1ccc(Cl)cc12] | 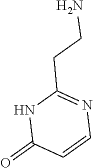 | Cmpd 142 | ¹H NMR (400 MHz, DMSO-d6) δ 8.00 (s, 1H), 7.83 (d, J = 6.7 Hz, 1H), 7.30-7.05 (m, 4H), 6.19 (d, J = 6.7 Hz, 1H), 4.76 (t, J = 6.9 Hz, 2H), 4.05- 3.62 (m, 4H), 3.18 (t, J = 6.9 Hz, 2H), 2.79- 2.61 (m, 3H), 2.52- 2.43 (m, 2H), 2.00- 1.60 (m, 3H), 1.33 (s, 1H), 1.17 (s, 3H). LCMS m/z 455.25 [M + H]⁺. |
| 184 | 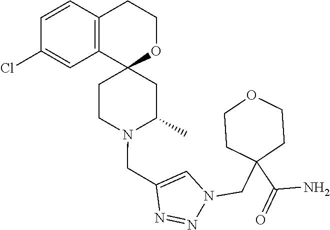 | 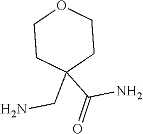 [SMILES: NCC1(C(N)=O)CCOCC1] | Cmpd 142 | ¹H NMR (400 MHz, DMSO-d6) δ 7.77 (s, 1H), 7.42 (s, 1H), 7.31- 7.05 (m, 4H), 4.52 (s, 2H), 4.00-3.84 (m, 1H), 3.83-3.41 (m, 7H), 2.78-2.59 (m, 3H), 2.58-2.52 (m, 2H), 2.00-1.82 (m, 3H), 1.72 (dd, J = 36.3, 12.4 Hz, 3H), 1.54-1.40 (m, 2H), 1.15 (d, J = 6.1 Hz, 3H). LCMS m/z |
| | | | | 474.30 [M + H]⁺. |
| 185 | 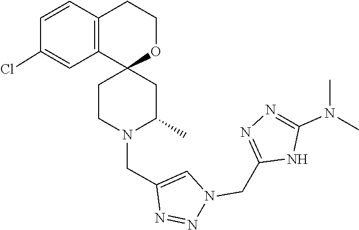 [SMILES: C[C@H]1C[C@@]2(CCN1Cc1cn(Cc3nnc(N(C)C)[nH]3)nn1)OCCc1ccc(Cl)cc12] | 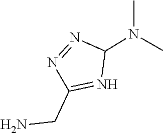 | Cmpd 142 | ¹H NMR (400 MHz, DMSO-d6) δ 7.98 (s, 1H), 7.25 (s, 1H), 7.21- 7.05 (m, 2H), 5.42 (s, 2H), 4.10-3.86 (m, 1H), 3.85-3.69 (m, 3H), 2.89 (s, 6H), 2.76- 2.63 (m, 3H), 2.56- 2.51 (m, 2H), 2.01- 1.56 (m, 4H), 1.17 (d, J = 5.9 Hz, 3H). LCMS m/z 457.28 [M + H]⁺. |
| 186 | 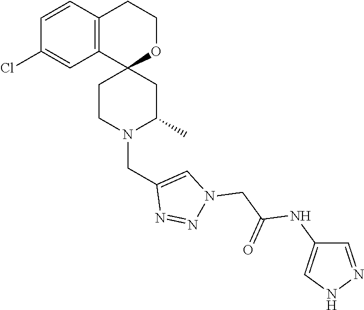 [SMILES: C[C@H]1C[C@@]2(CCN1Cc1cn(CC(=O)Nc3cn[nH]c3)nn1)OCCc1ccc(Cl)cc12] | 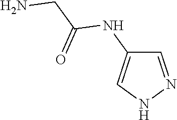 | Cmpd 142 | LCMS m/z 456.19 [M + H]⁺. |
| 187 | 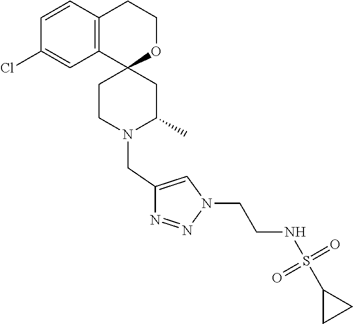 [SMILES: C[C@H]1C[C@@]2(CCN1Cc1cn(CCNS(=O)(=O)C3CC3)nn1)OCCc1ccc(Cl)cc12] | 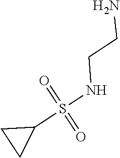 | Cmpd 142 | LCMS m/z 480.18 [M + H]⁺. |
| 188 | 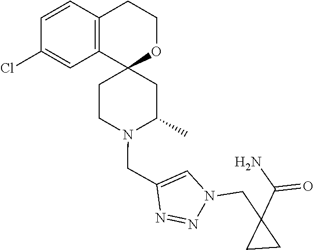 [SMILES: C[C@H]1C[C@@]2(CCN1Cc1cn(CC3(C(N)=O)CC3)nn1)OCCc1ccc(Cl)cc12] | 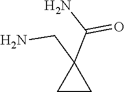 | Cmpd 142 | LCMS m/z 430.20 [M + H]⁺. |
| 189 | 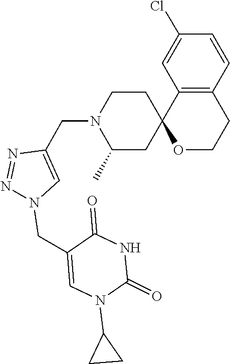 [SMILES: C[C@H]1C[C@@]2(CCN1Cc1cn(Cc3cn(C4CC4)c(=O)[nH]c3=O)nn1)OCCc1ccc(Cl)cc12] | 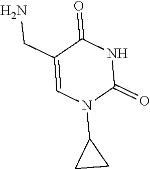 [SMILES: NCc1cn(C2CC2)c(=O)[nH]c1=O] | Cmpd 142 | LCMS m/z 497.21 [M + H]⁺. |

¹Stereoisomer was purified from product mixture by chiral SFC

### Compound 190

### N-methyl-1′-[(1-phenylpyrazol-4-yl)methyl]spiro[1H-isobenzofuran-3,4′-piperidine]-1-carboxamide (190)

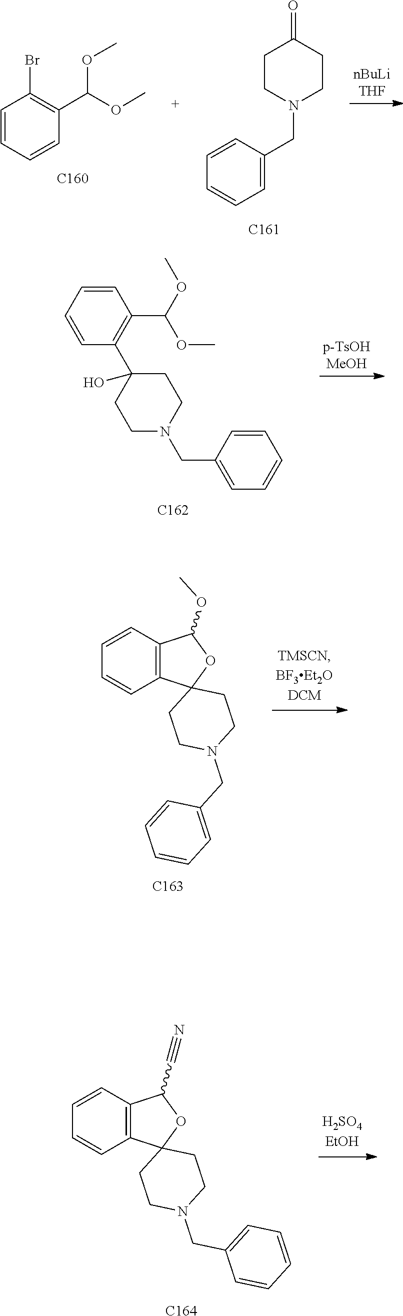

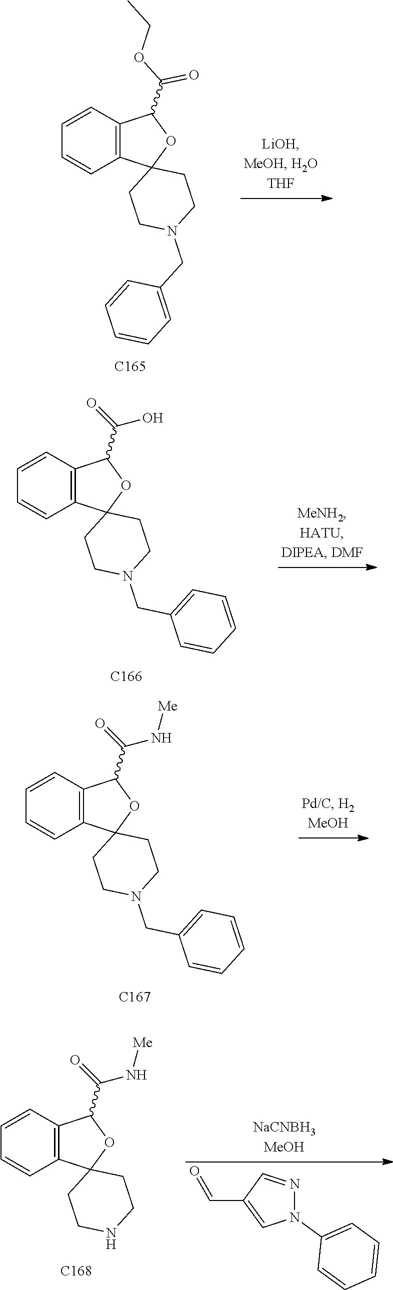

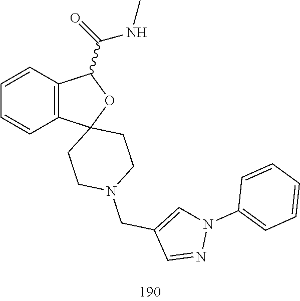 [SMILES: CNC(=O)C1OC2(CCN(Cc3cnn(-c4ccccc4)c3)CC2)c2ccccc21]

### Step 1. Synthesis of 1-benzyl-4-[2-(dimethoxymethyl)phenyl]piperidin-4-ol (C162)

To a stirred solution of 1-bromo-2-(dimethoxymethyl)benzene (5 g, 0.0214 mol) in THF (60 mL) was added n-BuLi (14 mL of 1.6 M, 0.0224 mol) at −78° C. over a period of 10 min under argon and stirred for 5 min. To the reaction mixture was added a solution of 1-benzylpiperidin-4-one (3.4 g, 0.0176 mol) in THF (10 mL) and stirred at −78° C. for 2 hours at which point the solution was allowed to warm to room temperature and stirred for 4 h. The reaction mixture was quenched with water at 0° C., diluted with sat. NH₄Cl solution, and extracted with EtOAc (2×50 mL). The organic layer was washed with brine, dried over Na₂SO₄ and concentrated under reduced pressure to yield 1-benzyl-4-[2-(dimethoxymethyl)phenyl]piperidin-4-ol (6.4 g, 35% yield) as pale yellow semi solid. LCMS m/z 342.08 [M+1]⁺.

### Step 2. Synthesis of 1′-benzyl-1-methoxy-spiro[1H-isobenzofuran-3,4′-piperidine](C163)

To a stirred solution of 1-benzyl-4-[2-(dimethoxymethyl)phenyl]piperidin-4-ol (6.4 g, 0.0075 mol) in methanol (50 mL) was added p-TsOH hydrate (4.5 g, 0.0232 mol) at room temperature and stirred for 5 days. The reaction mixture was diluted with 2 M NaOH sol. to pH ˜10 and compound was extracted with DCM (2×200 mL). The organic layer was washed with brine, dried over Na₂SO₄ and concentrated under reduced pressure. The residue was purified by column chromatography (Silica, 230-400) and eluted at 25% EtOAc in pet ether. The collected fractions were concentrated under reduced pressure to yield 1′-benzyl-1-methoxy-spiro[1H-isobenzofuran-3,4′-piperidine](1.9 g, 59% yield) as yellow liquid. ¹H NMR (300 MHz, Chloroform-d) δ 7.42-7.28 (m, 8H), 7.18 (d, J=7.4 Hz, 1H), 6.07 (s, 1H), 3.60 (s, 2H), 3.47 (s, 3H), 2.92-2.85 (m, 2H), 2.47 (t, J=10.8 Hz, 2H), 2.15-1.98 (m, 2H), 1.81 (dd, J=13.5 Hz, 2.7 Hz, 1H), 1.64 (dd, J=13.8 Hz, 2.7 Hz, 1H). LCMS m/z 310.38 [M+1]⁺.

### Step 3. Synthesis of 1′-benzylspiro[1H-isobenzofuran-3,4′-piperidine]-1-carbonitrile (C164)

To a stirred solution of 1′-benzyl-1-methoxy-spiro[1H-isobenzofuran-3,4′-piperidine](1.9 g, 0.0044 mol) in DCM (100 mL) was added TMSCN (3.8857 g, 5 mL, 0.0384 mol) and BF₃·OEt₂ (1.0143 g, 0.9 mL, 0.0070 mol) at −25 C and stirred for 30 min. Then allowed to warm to 0° C. and stirred for 1 h. The reaction mixture was quenched with methanol (1 mL) followed by 2 M NaOH sol. The organic layer was separated, dried over Na₂SO₄ and concentrated under reduced pressure. The residue was purified by column chromatography (Silica, 230-400) and eluted at 40% EtOAc in pet ether. The collected fractions were concentrated under reduced pressure to yield 1′-benzylspiro[1H-isobenzofuran-3,4′-piperidine]-1-carbonitrile (1.4 g, 97% yield) as an off-white semi solid. ¹H NMR (400 MHz, Chloroform-d) 6 7.43-7.25 (m, 8H), 7.20 (d, J=6 Hz, 1H), 5.87 (s, 1H), 3.59 (s, 2H), 2.88-2.82 (m, 2H), 2.53-2.38 (m, 2H), 2.10-1.91 (m, 3H), 1.73-1.69 (m, 1H). LCMS m/z 305.33 [M+1]⁺.

### Step 4. Synthesis of ethyl 1′-benzylspiro[1H-isobenzofuran-3,4′-piperidine]-1-carboxylate (C165)

To a stirred solution of 1′-benzylspiro[1H-isobenzofuran-3,4′-piperidine]-1-carbonitrile (100 mg, 295 μmol) in EtOH (2 mL) was added H₂SO₄ (552.00 mg, 0.3000 mL, 0.0055 mol) and two drops of water at room temperature. The reaction mixture was stirred at 90° C. for 20 hours. The reaction mixture was cooled to room temperature, diluted with chilled water (10 mL) and extracted with EtOAc (2×20 mL). The organic layer was washed with 2 M NaOH sol. (5 mL), brine, dried over Na₂SO₄ and concentrated under reduced pressure. The residue was purified by column chromatography (Silica, 230-400) and eluted at 30% EtOAc in pet ether. The collected fractions were concentrated under reduced pressure to yield ethyl 1′-benzylspiro[1H-isobenzofuran-3,4′-piperidine]-1-carboxylate (70 mg, 67% yield) as a pale yellow thick liquid. ¹H NMR (400 MHz, Chloroform-d) δ 7.41-7.26 (m, 8H), 7.19 (d, J=6.8 Hz, 1H), 5.66 (s, 1H), 4.22 (q, J=3.2 Hz, 2H), 3.60 (s, 2H), 2.86 (t, J=6.4 Hz, 2H), 2.62-2.46 (m, 2H), 2.11-1.97 (m, 3H), 1.76 (d, J=13.6 Hz, 1H), 1.28 (t, J=7.2 Hz, 3H). LCMS m/z 352.08 [M+1]⁺.

### Step 5. Synthesis of 1′-benzylspiro[1H-isobenzofuran-3,4′-piperidine]-1-carboxylic acid (C166)

To a stirred solution of ethyl 1′-benzylspiro[1H-isobenzofuran-3,4′-piperidine]-1-carboxylate (0.9 g, 0.0024 mol) in THF (20 mL) was added the solution of LiOH hydrate (302 mg, 0.0071 mol) in water (6 mL) at room temperature. Then to the reaction mixture was added methanol (3 mL) and stirred at room temperature for 16 hours. The reaction mixture was concentrated under reduced pressure to afford crude residue. It was diluted with water (50 mL), acidified with 1 M HCl sol. at 0° C. and compound was extracted with 10% MeOH in DCM (2×100 mL). The organic layer was dried over Na₂SO₄ and concentrated under reduced pressure to get 1′-benzylspiro[1H-isobenzofuran-3,4′-piperidine]-1-carboxylic acid (780 mg, 99% yield) as an off white solid. ¹H NMR (300 MHz, DMSO-d6) δ 7.61 (d, J=4.2 Hz, 2H), 7.47-7.39 (m, 6H), 7.20 (br s, 1H), 5.66 (s, 1H), 4.31 (s, 2H), 3.29-3.14 (m, 2H), 2.43-2.20 (m, 2H), 1.98-1.90 (m, 2H). LCMS m/z 324.28 [M+1]⁺.

### Step 6 Synthesis of 1′-benzyl-N-methyl-spiro[1H-isobenzofuran-3,4′-piperidine]-1-carboxamide (C167)

To a stirred solution of 1′-benzylspiro[1H-isobenzofuran-3,4′-piperidine]-1-carboxylic acid (780 mg, 0.0024 mol), HATU (1.13 g, 0.0029 mol) and methyl amine (2M in THF) (2.4000 mL of 2 M, 0.0048 mol) in DMF (10 mL) was added DIPEA (1.5270 g, 2.1 mL, 0.0116 mol) at room temperature and stirred for 16 hours. The reaction mass was diluted with chilled water and compound was extracted with 5% MeOH in DCM (2×50 mL). The organic layer was dried over Na₂SO₄ and concentrated under reduced pressure to get 1′-benzyl-N-methyl-spiro[1H-isobenzofuran-3,4′-piperidine]-1-carboxamide (820 mg, 84% yield) as thick brown liquid. LCMS m/z 337.11 [M+1]⁺.

### Step 7. Synthesis of N-methylspiro[1H-isobenzofuran-3,4′-piperidine]-1-carboxamide (C168)

A suspension of 1′-benzyl-N-methyl-spiro[1H-isobenzofuran-3,4′-piperidine]-1-carboxamide (100 mg, 243.74 μmol) and 10% Palladium on carbon (25 mg, 10% w/w, 23 μmol) in methanol (5 mL) was hydrogenated under balloon pressure at room temperature for 16 hours. The reaction mixture was filtered through a pad of celite and washed with MeOH (20 mL). The filtrates were concentrated under reduced pressure. The resulting residue was purified by reversed-phase chromatography (Column: C18. Gradient: 0-100% 1:1 MeCN:MeOH in water with 10 mM NH₄HCO₃). The collected pure fractions were concentrated under reduced pressure to yield N-methylspiro[1H-isobenzofuran-3,4′-piperidine]-1-carboxamide (16 mg, 26% yield) as an off white gum. ¹H NMR (400 MHz, DMSO-d6): δ 7.55 (d, J=7.6 Hz, 1H), 7.38 (d, J=7.6 Hz, 1H), 7.38-7.26 (m, 2H), 7.22 (d, J=7.6 Hz, 1H), 5.39 (s, 1H), 3.02 (t, J=12.4 Hz, 1H), 2.99-2.84 (m, 3H), 2.63 (d, J=4.8 Hz, 3H), 1.92-1.84 (m, 1H), 1.88-1.74 (m, 2H), 1.67 (d, J=4.8 Hz, 1H). LCMS m/z 247.1 [M+1]⁺.

### Step 8. Synthesis of N-methyl-1′-[(1-phenylpyrazol-4-yl)methyl]spiro[1H-isobenzofuran-3,4′-piperidine]-1-carboxamide (190)

A solution of N-methylspiro[1H-isobenzofuran-3,4′-piperidine]-1-carboxamide (200 mg, 649.60 μmol), 1-phenylpyrazole-4-carbaldehyde (137 mg, 779.75 μmol), and acetic acid (5 drops) in methanol (5 mL) was stirred at room temperature for 16 hours. Then to the reaction mixture was added NaCNBH₃ (50 mg, 779.73 μmol) at 0° C., gradually allowed to warm to room temperature and stirred for 6 hours. The reaction mass was concentrated under reduced pressure to get crude compound. It was purified by reversed-phase chromatography (Column: C18. Gradient: 0-100% 1:1 MeOH:MeCN in water with 10 mM NH₄HCO₃). The collected fractions were concentrated under reduced pressure to yield N-methyl-1′-[(1-phenylpyrazol-4-yl)methyl]spiro[1H-isobenzofuran-3,4′-piperidine]-1-carboxamide (50 mg, 19% yield) as an off white solid. ¹H NMR (400 MHz, DMSO-d6): δ 8.42 (s, 1H), 7.83 (d, J=7.6 Hz, 2H), 7.68 (s, 1H), 7.54-7.46 (m, 3H), 7.38-7.25 (m, 5H), 5.39 (s, 1H), 3.52 (s, 2H), 2.81 (t, J=10 Hz, 2H), 2.62 (d, J=4.8 Hz, 3H), 2.54 (s, 1H), 2.34 (t, J=10 Hz, 1H), 2.09-2.03 (m, 1H), 1.87-1.82 (m, 2H), 1.64 (d, J=11.6 Hz, 1H). LCMS m/z 403.2 [M+1]⁺.

### Preparation S20

### 1-[(1-phenylpyrazol-4-yl)methyl]piperidin-4-one (S20)

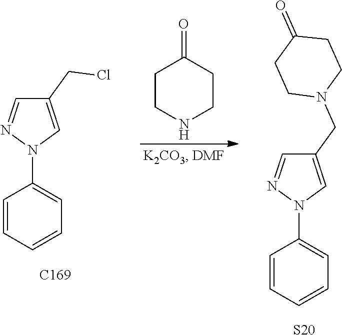 [SMILES: "C169" ; ClCc1cnn(-c2ccccc2)c1 ; O=C1CCNCC1 ; O=C1CCN(Cc2cnn(-c3ccccc3)c2)CC1 ; "S20"]

To a solution of 4-(chloromethyl)-1-phenyl-pyrazole (8.3 g, 0.0388 mol) in DMF (80 mL) was added piperidin-4-one (Hydrochloric Acid (1)) (7.5 g, 0.0387 mol) followed by K₂CO₃ (14 g, 0.100 mol) at room temperature and stirred at 100° C. for 16 hours. The reaction mixture was quenched with ice/water (250 mL) and extracted with ethyl acetate (2×500 mL), washed with brine (250 mL) and dried over sodium sulfate, and concentrated under reduced pressure. The crude residue was purified by flash column chromatography with 60% ethyl acetate in hexane, pure fractions were concentrated under reduced pressure to get 1-[(1-phenylpyrazol-4-yl)methyl]piperidin-4-one (9.1 g, 85% yield) as a solid. ¹H NMR (400 MHz, Chloroform-d) δ 7.89 (s, 1H), 7.69-7.66 (m, 3H), 7.47-7.42 (m, 2H), 7.31-7.26 (m, 1H), 3.61 (s, 2H), 2.79 (t, J=6.0 Hz, 4H), 2.47 (t, J=6.0 Hz, 4H). LCMS m/z 256.13 [M+1]⁺.

### Preparation S21

### 1-3-trimethylsilylprop-2-ynyl)piperidin-4-one (S21)

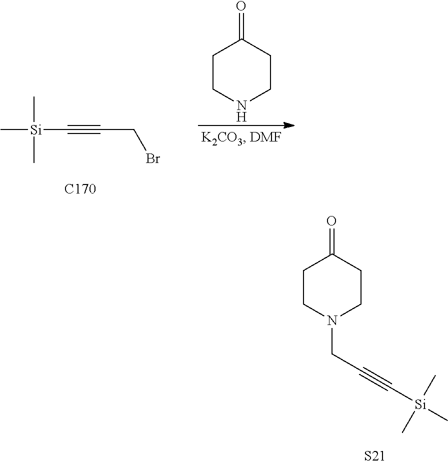

To a stirred solution of piperidin-4-one (Hydrochloric Acid (1)) (1 g, 0.0066 mol) in MeCN (10 mL) was added K₂CO₃ (3.0 g, 0.0215 mol) followed by 3-bromoprop-1-ynyl(trimethyl)silane (1.5213 g, 1.3003 mL, 0.0078 mol) at 0° C. and stirred for 4 h at room temperature. The reaction was diluted with water (50 mL) and extracted with ethyl acetate (2×50 mL). Organic layer was dried over sodium sulfate and concentrated under reduced pressure to afford crude 1-(3-trimethylsilylprop-2-ynyl)piperidin-4-one (850 mg, 90% pure, 55% yield) ¹H NMR (300 MHz, Chloroform-d) δ: 3.44 (s, 2H), 2.85 (t, J=6.3 Hz, 4H), 2.50 (t, J=5.7 Hz, 4H), 0.16 (s, 9H). as a colorless liquid.

### Preparation S22

### 1-[[1-(2-pyridyl)pyrazol-4-yl]methyl]piperidin-4-one (S22)

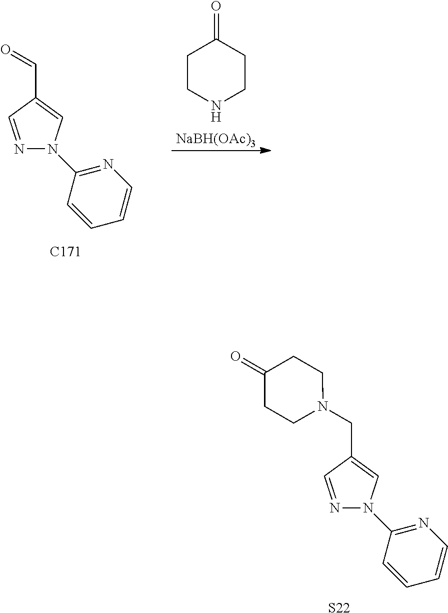

To a stirred solution of piperidin-4-one (2.8 g, 26.833 mmol) in 1,2-dichloroethane (40 mL), MeOH (10 mL) was added 1-(2-pyridyl)pyrazole-4-carbaldehyde (3.5 g, 19.807 mmol) followed by Sodium triacetoxyborohydride (9 g, 41.615 mmol) at 0° C. and stirred for 1 h at room temperature. The reaction was diluted with ethyl acetate, filtered, and concentrated under reduced pressure. The crude residue was purified by flash column chromatography with ethyl acetate, pure fraction were concentrated under reduced pressure to get 1-[[1-(2-pyridyl)pyrazol-4-yl]methyl]piperidin-4-one (2.5 g, 37% yield) as yellow sticky gum. ¹H NMR (400 MHz, Chloroform-d) δ 8.50 (s, 1H), 8.41-8.39 (m, 1H), 7.97-7.95 (m, 1H), 7.84-7.79 (m, 1H), 7.71 (s, 1H), 7.20-7.17 (m, 1H), 3.65 (s, 2H), 2.81 (t, J=6.0 Hz, 4H), 2.49 (t, J=6.4 Hz, 4H). LCMS m/z 257.24 [M+1]⁺.

### Preparation S23

### 1-[(1-tetrahydropyran-4-ylpyrazol-4-yl)methyl]piperidin-4-one (S23)

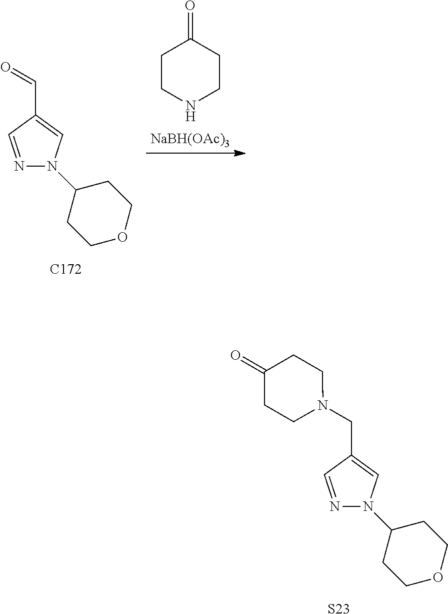

1-[(1-tetrahydropyran-4-ylpyrazol-4-yl)methyl]piperidin-4-one (S23) was prepared using the same method as used to prepare S22 with starting aldehyde C172.

### Preparation S24

### 1-[(1-tetrahydropyran-2-ylpyrazol-4-yl)methyl]piperidin-4-one (S24)

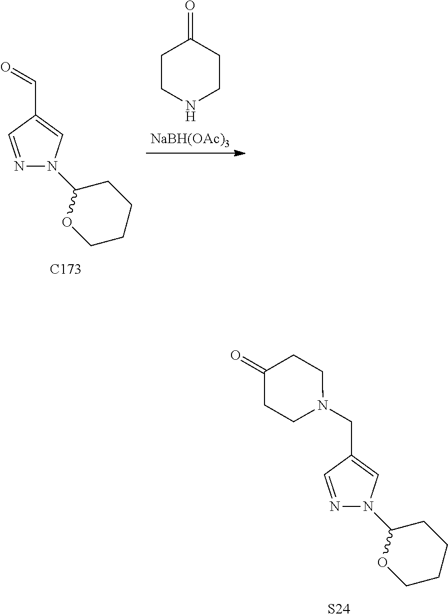

1-[(1-tetrahydropyran-2-ylpyrazol-4-yl)methyl]piperidin-4-one (S24) was prepared using the same method as used to prepare S22 with starting aldehyde C173.

### Preparation S25

### 2-bromo-1-(dimethoxymethyl)-4-methyl-benzene (S25)

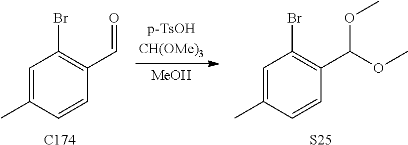

To a stirred solution of 2-bromo-4-methyl-benzaldehyde (5 g, 0.0226 mol) in MeOH (80 mL) was added p-TsOH (783 mg, 0.7318 mL, 0.0045 mol) followed by Trimethyl orthoformate (5.9539 g, 6.2 mL, 0.0555 mol) at room temperature, the reaction mixture was stirred at 75° C. for 15 h. The reaction was diluted with sat. sodium bicarbonate solution (100 mL), methanol removed under reduced pressure. The aqueous layer was extracted with ethyl acetate (2×100 mL), dried over sodium sulfate and concentrated under reduced pressure to yield crude 2-bromo-1-(dimethoxymethyl)-4-methyl-benzene (5.2 g, 86% yield) as a pale yellow liquid which was used without additional purification. ¹H NMR (400 MHz, Chloroform-d) δ ppm: 7.47 (d, J=8 Hz, 1H), 7.37 (s, 1H), 7.13-7.10 (m, 1H), 5.53 (s, 1H), 3.36 (s, 6H), 2.30 (s, 3H).

### Preparation S26

### 2-bromo-I-(dimethoxymethyl)-4-(trifluoromethyl)benzene (S26)

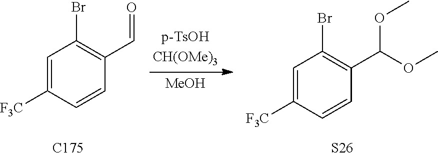 [SMILES: "MeOH" ; O=Cc1ccc(C(F)(F)F)cc1Br ; COC(OC)c1ccc(C(F)(F)F)cc1Br ; "C175" ; "S26"]

2-bromo-1-(dimethoxymethyl)-4-(trifluoromethyl)benzene (S26) was prepared using the same method used to synthesize S25 with starting aldehyde C175.

### Preparation S27

### 1-[[1-(3-pyridyl)pyrazol-4-yl]methyl]piperidin-4-one (S27)

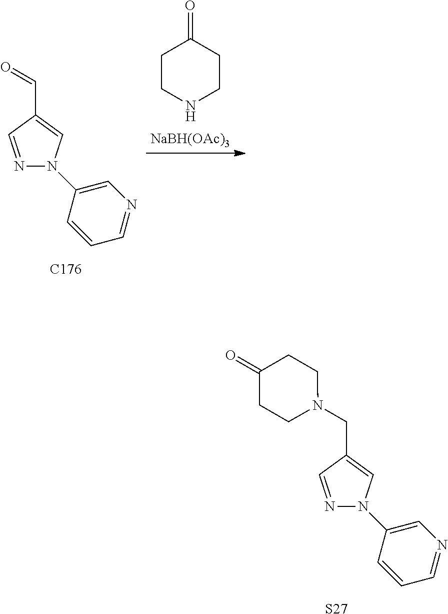

1-[[1-(3-pyridyl)pyrazol-4-yl]methyl]piperidin-4-one (S27) was prepared using the same method as used to prepare S22 with starting aldehyde C176.

### Preparation S28

### 5-chloro-1′-[(1-phenylpyrazol-4-yl)methyl]spiro[1H-isobenzofuran-3,4′-piperidine]-1-carbonitrile (S28)

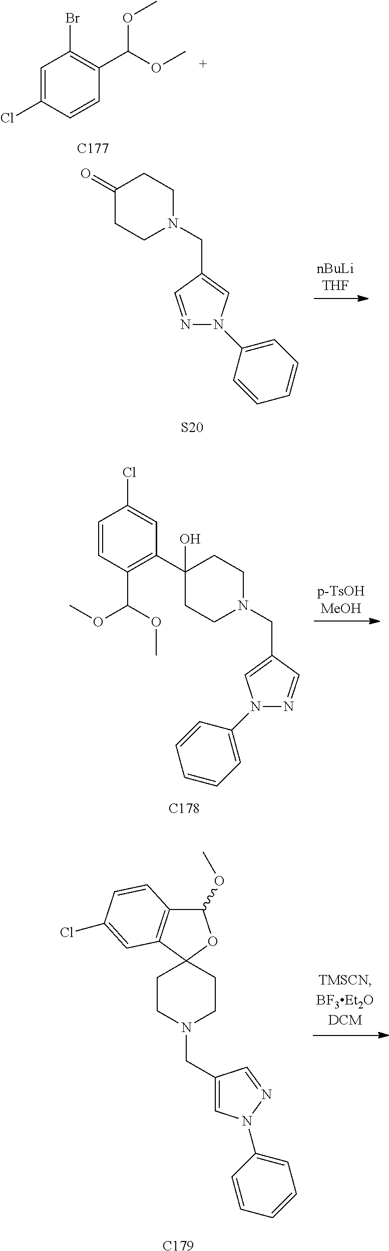

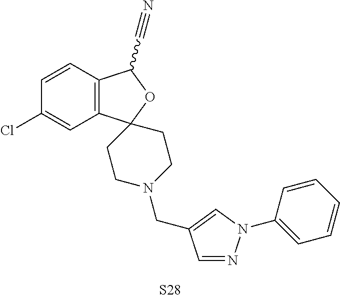 [SMILES: N#CC1OC2(CCN(Cc3cnn(-c4ccccc4)c3)CC2)c2cc(Cl)ccc21 ; "S28"]

### Step 1. Synthesis of 4-[5-chloro-2-(dimethoxymethyl)phenyl]-1-[(1-phenylpyrazol-4-yl)methyl]piperidin-4-ol (C178)

To a stirred solution of 2-bromo-4-chloro-1-(dimethoxymethyl)benzene (5.0 g, 0.0169 mol) and 1-[(1-phenylpyrazol-4-yl)methyl]piperidin-4-one (5.0 g, 0.0180 mol) in THF (100 mL) was added n-Butyllithium (10 mL of 2.5 M, 0.0250 mol) at −80° C. and stirred for 2h, The reaction slowly warmed to room temperature and stirred for 4 h. The reaction was quenched with water (100 mL) and extracted with ethyl acetate (2×250 mL). Organic layer was dried over sodium sulfate and concentrated under reduced pressure to yield crude 4-[5-chloro-2-(dimethoxymethyl)phenyl]-1-[(1-phenylpyrazol-4-yl)methyl]piperidin-4-ol (11 g, 86% yield, -60% pure by LC/MS) as a yellow liquid which was used for successive steps without further

### Step 2. Synthesis of 5-chloro-1-methoxy-1′-[(1-phenylpyrazol-4-yl)methyl]spiro[1H-isobenzofuran-3,4′-piperidine](C179)

To a stirred solution of 4-[5-chloro-2-(dimethoxymethyl)phenyl]-1-[(1-phenylpyrazol-4-yl)methyl]piperidin-4-ol (10 g, -40% pure by LC/MS, 9.0508 mmol) in MeOH (100 mL) was added p-TsOH (9.2 g, 8.5981 mL, 52.357 mmol) at 0° C. The reaction was allowed to warm to room temperature and stirred for 16 h. The reaction mixture was directly concentrated under reduced pressure to a crude residue which was dissolved in DCM (200 mL) and washed with 1 M NaOH solution (50 mL). The organic layer dried over sodium sulfate, filtered, and concentrated under vacuum. The resulting crude residue was purified by silica gel chromatography (Gradient: 0-50% EtOAc in hexane) to afford 5-chloro-1-methoxy-1′-[(1-phenylpyrazol-4-yl)methyl]spiro[1H-isobenzofuran-3,4′-piperidine](4 g, ˜80% pure by LC/MS, 86% yield) as a brown gum. ¹H NMR (300 MHz, Chloroform-d): δ 7.91 (s, 1H), 7.70-7.68 (m, 3H), 7.47-7.41 (m, 2H), 7.28-7.24 (m, 2H), 7.14 (s, 1H), 6.01 (s, 1H), 3.58 (s, 2H), 3.46 (s, 3H), 2.96-2.87 (m, 2H), 2.52-2.44 (m, 2H), 2.12-2.06 (m, 2H), 2.04-1.94 (m, 1H), 1.86-1.80 (m, 1H), 1.69-1.64 (m, 1H). LCMS m/z 410.06 [M+1]⁺

### Step 3. Synthesis of 5-chloro-1′-[(1-phenylpyrazol-4-yl)methyl]spiro[1H-isobenzofuran-3,4′-piperidine]-1-carbonitrile (S28)

To a stirred solution of 5-chloro-1-methoxy-1′-[(1-phenylpyrazol-4-yl)methyl]spiro[1H-isobenzofuran-3,4′-piperidine](2 g, 0.0047 mol) in DCM (30 mL) was added TMSCN (3.7302 g, 4.7039 mL, 0.0368 mol) followed by BF₃·OEt₂ (1.3341 g, 1.1601 mL, 0.0093 mol) at −25° C. and stirred for 30 min. The reaction was then allowed to warm to 0° C. and stirred for 1 h. The reaction mixture was quenched with MeOH (10 mL) and water (100 mL) and then extracted with DCM (2×250 mL). The combined organic layer was dried over sodium sulfate and concentrated under reduced pressure to afford 5-chloro-1′-[(1-phenylpyrazol-4-yl)methyl]spiro[1H-isobenzofuran-3,4′-piperidine]-1-carbonitrile (1.8 g, 95%), ¹H NMR (300 MHz, Chloroform-d): δ 7.90 (s, 1H), 7.71-7.68 (m, 3H), 7.48-7.42 (m, 2H), 7.39-7.26 (m, 3H), 7.17 (s, 1H), 5.82 (s, 1H), 3.59 (s, 2H), 2.94 (brs, 2H), 2.46-2.43 (m, 2H), 2.06-1.99 (m, 3H), 1.77-1.72 (in, 1H). LCMS m/z 405.02 [M+1]⁺.

### Preparation S29-S36

Intermediate compounds S29-S36 (see Table 14) were prepared from the appropriate aryl bromides and appropriate piperidones employing the same method as described for S28. All aryl bromides and piperidones were obtained from commercial sources or were synthesized as described above.

TABLE 14
Structure and physicochemical data for intermediates S29-S36

| | | | | ¹H NMR; |
|---|---|---|---|---|
| | | | | LCMS m/z |
| Cmpd | Product | Aryl Bromide | Piperidone | [M + H]⁺ |
| S29 | 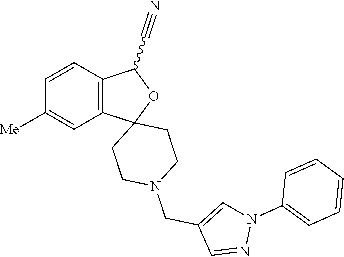 [SMILES: Cc1ccc2c(c1)C1(CCN(Cc3cnn(-c4ccccc4)c3)CC1)OC2C#N] | 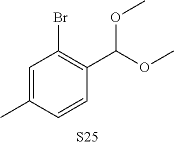 | 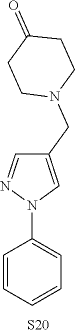 [SMILES: O=C1CCN(Cc2cnn(-c3ccccc3)c2)CC1 ; "S20"] | ¹H NMR (300 MHz, DMSO-d6) δ ppm: 8.60 (s, 1H), 7.89- 7.82 (m, 3H), 7.57-7.52 (m, 2H), 7.45- 7.30 (m, 3H), 7.16 (brs, 1H), 6.24 (s, 1H), 5.72 (s, 1H), 4.22 (brs, 2H), 3.40 (brs, 2H), 3.17- 3.11 (m, 3H), 2.38 (s, 3H), 2.23-2.09 (m, 2H). LCMS |
| | | | | m/z 385.5 |
| | | | | [M + 1]⁺. |
| S30 | 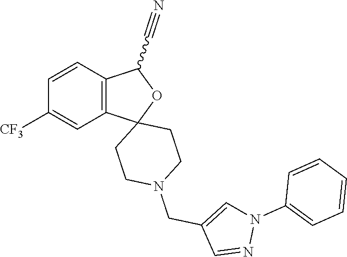 [SMILES: N#CC1OC2(CCN(Cc3cnn(-c4ccccc4)c3)CC2)c2cc(C(F)(F)F)ccc21] | 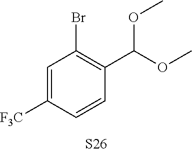 [SMILES: COC(OC)c1ccc(C(F)(F)F)cc1Br ; "S26"] | 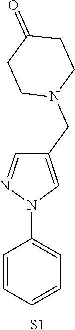 | ¹H NMR (400 MHz, DMSO- d6) δ 8.42 (s, 1H), 7.90 (s, 1H), 7.84- 7.77 (m, 4H), 7.68 (s, 1H), 7.48 (t, J = 8.0 Hz, 2H), 7.30 (t, J = 7.2 Hz, 1H), 6.34 (s, 1H), 3.52 (s, 2H), 2.86 (t, J = 8.0 Hz, 2H), 2.38-2.30 (m, 2H), 2.21- 2.07 (m, 2H), 1.79-1.75 (m, 1H), 1.67- |
| | | | | 1.63 (m, 1H). |
| | | | | 439.3 LCMS |
| | | | | m/z [M + 1]⁺. |
| S31 | 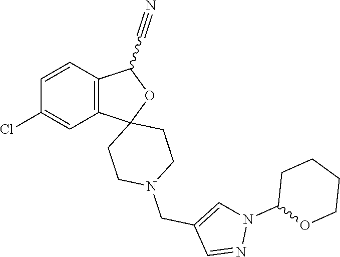 [SMILES: N#CC1OC2(CCN(Cc3cnn(C4CCCCO4)c3)CC2)c2cc(Cl)ccc21] | 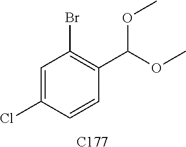 | 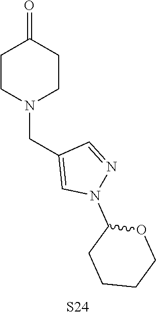 [SMILES: O=C1CCN(Cc2cnn(C3CCCCO3)c2)CC1 ; "S24"] | LCMS m/z 413.28 [M + 1]⁺. |
| S32 | 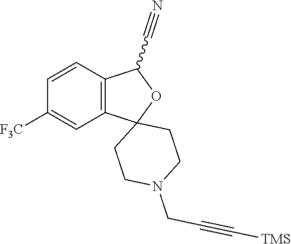 [SMILES: C[Si](C)(C)C#CCN1CCC2(CC1)OC(C#N)c1ccc(C(F)(F)F)cc12] | 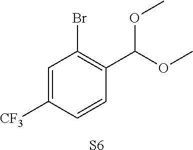 | 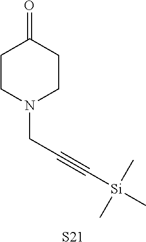 | ¹H NMR (Chloroform- d, 400 MHz): δ 7.70-7.69 (m, 1H), 7.55- 7.53 (m, 1H), 7.43 (brs, 1H), 5.91 (s, 1H), 3.35 (s, 2H), 2.95- 2.91 (m, 2H), 2.66-2.53 (m, 2H), 2.16- 2.02 (m, 4H), 0.19-0.16 (m, |
| | | | | 9H). LCMS |
| | | | | m/z 393.64 |
| | | | | [M + 1]⁺ |
| S33 | 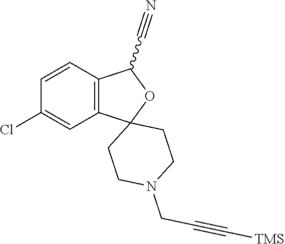 [SMILES: C[Si](C)(C)C#CCN1CCC2(CC1)OC(C#N)c1ccc(Cl)cc12] | 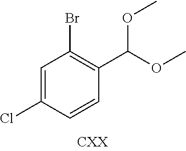 | 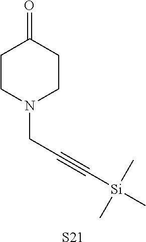 | LCMS m/z 359.32 [M + 1]⁺ |
| S34 | 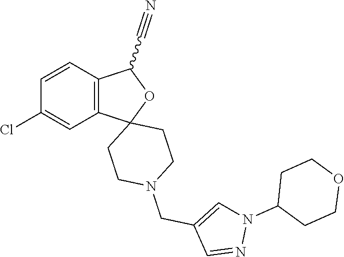 [SMILES: N#CC1OC2(CCN(Cc3cnn(C4CCOCC4)c3)CC2)c2cc(Cl)ccc21] | 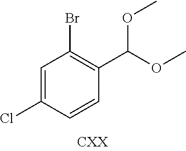 | 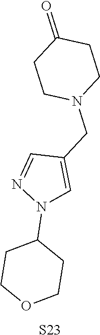 [SMILES: O=C1CCN(Cc2cnn(C3CCOCC3)c2)CC1 ; "S23"] | LCMS m/z 413.41 [M + 1]⁺ |
| S35 | 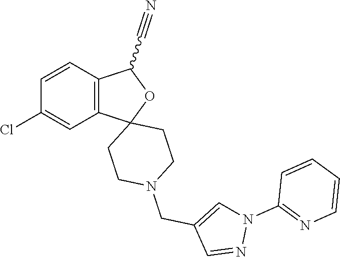 [SMILES: N#CC1OC2(CCN(Cc3cnn(-c4ccccn4)c3)CC2)c2cc(Cl)ccc21] | 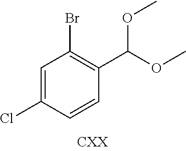 | 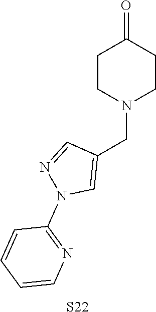 [SMILES: O=C1CCN(Cc2cnn(-c3ccccn3)c2)CC1 ; "S22"] | LCMS m/z 406.49 [M + 1]⁺ |
| S36 | 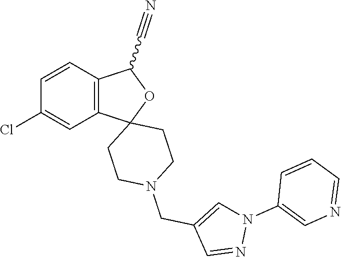 [SMILES: N#CC1OC2(CCN(Cc3cnn(-c4cccnc4)c3)CC2)c2cc(Cl)ccc21] | 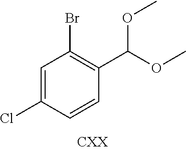 | 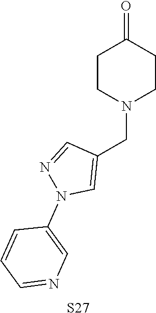 [SMILES: O=C1CCN(Cc2cnn(-c3cccnc3)c2)CC1 ; "S27"] | LCMS m/z 406.17 [M + 1]⁺ |

### Compound 191

### 5-chloro-1′-[(1-phenylpyrazol-4-yl)methyl]spiro[1H-isobenzofuran-3,4′-piperidine]-1-carboxamide (191)

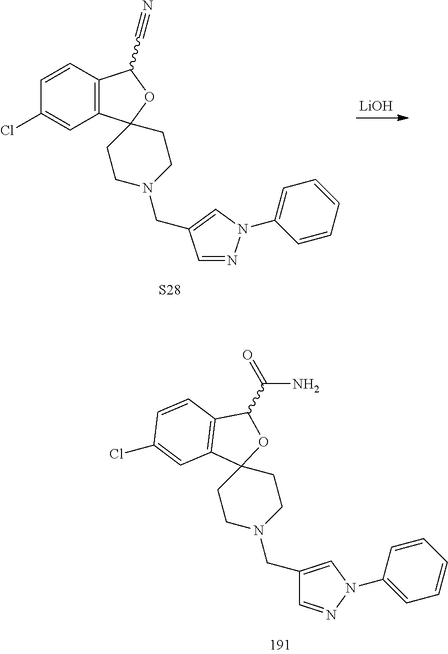 [SMILES: N#CC1OC2(CCN(Cc3cnn(-c4ccccc4)c3)CC2)c2cc(Cl)ccc21 ; "LiOH" ; "S28" ; NC(=O)C1OC2(CCN(Cc3cnn(-c4ccccc4)c3)CC2)c2cc(Cl)ccc21]

To a stirred solution of 5-chloro-1′-[(1-phenylpyrazol-4-yl)methyl]spiro[1H-isobenzofuran-3,4′-piperidine]-1-carbonitrile (200 mg, 454.39 mol) (S9) in Water (1.0 mL) and THF (1.0 mL) was added LiOH (Water (1)) (40 mg, 943.68 μmol) at room temperature. The reaction mixture was stirred for 16 h. The reaction was diluted with water (20 mL) and extracted with ethyl acetate (3×50 mL). The organic layer was dried over sodium sulfate and concentrated under reduced pressure. The crude residue purified by reversed-phase chromatography (Column: C18. Gradient: 0-100% MeCN in water with 0.1% formic acid) to afford 5-chloro-1′-[(1-phenylpyrazol-4-yl)methyl]spiro[1H-isobenzofuran-3,4′-piperidine]-1-carboxamide (Formic Acid (1)) (30 mg, 14%) as a white solid. ¹H NMR (400 MHz, DMSO-d6) δ: 8.41 (s, 1H), 8.18 (s, 1H), 7.83 (d, J=8.8 Hz, 2H), 7.67 (s, 1H), 7.48 (t, J=8.4 Hz, 2H), 7.4-7.33 (m, 4H), 7.28 (t, J=7.2 Hz, 1H), 7.12 (brs, 1H), 5.35 (s, 1H), 3.55 (s, 2H), 2.81-2.61 (m, 2H), 2.46 (brs, 1H), 2.37-2.32 (m, 1H), 2.10-2.03 (m, 1H), 1.92-1.88 (m, 2H), 1.65-1.04 (m, 1H). LCMS m/z 423.05 [M+1]⁺.

### 5-chloro-1′-[(1-phenylpyrazol-4-yl)methyl]spiro[1H-isobenzofuran-3,4′-piperidine]-1-carboxylic acid (192), 5-chloro-1′-[(1-phenylpyrazol-4-yl)methyl]spiro[1H-isobenzofuran-3,4′-piperidine]-1-carboxamide (193), and 5-chloro-1′-[(1-phenylpyrazol-4-yl)methyl]spiro[1H-isobenzofuran-3,4′-piperidine]-1-carboxamide (194)

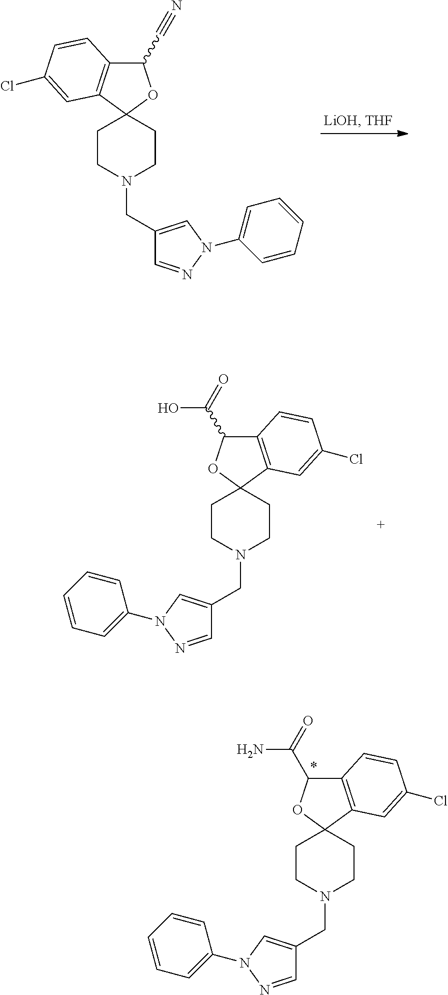 [SMILES: N#CC1OC2(CCN(Cc3cnn(-c4ccccc4)c3)CC2)c2cc(Cl)ccc21 ; O=C(O)C1OC2(CCN(Cc3cnn(-c4ccccc4)c3)CC2)c2cc(Cl)ccc21 ; NC(=O)C1OC2(CCN(Cc3cnn(-c4ccccc4)c3)CC2)c2cc(Cl)ccc21]

To a stirred solution of 5-chloro-1′-[(1-phenylpyrazol-4-yl)methyl]spiro[1H-isobenzofuran-3,4′-piperidine]-1-carbonitrile (200 mg, 454.39 mol) in Water (1.0 mL) and THF (1.0 mL) was added LIOH (40 mg, 943.68 μmol) at ambient temperature. The reaction mixture was stirred for 16 hours, then the reaction mass was diluted with water (20 mL), and
extracted with ethyl acetate (3×50 mL). Organic layer was dried over sodium sulfate and concentrated (aqueous layer set aside for purification of carboxylic acid), then purified by reverse phase HPLC (Gradient: 45-99% MeCN in 0.1% aqueous formic acid) to provide 5-chloro-1′-[(1-phenylpyrazol-4-yl)methyl]spiro[1H-isobenzofuran-3,4′-piperidine]-1-carboxamide as a mixture of enantiomers. Separation of stereoisomers by chiral SFC purification (Column: AS-H, 10×250 mm; Gradient: 60% CO₂ in methanol (5 mM Methanolic ammonia); Flow rate: 15 mL/min) provided the first eluting peak (193 5-chloro-1′-[(1-phenylpyrazol-4-yl)methyl]spiro[1H-isobenzofuran-3,4′-piperidine]-1-carboxamide (6.3 mg, 79%). ¹H NMR (300 MHz, Chloroform-d) δ 8.26 (s, 1H), 7.83-7.70 (m, 3H), 7.55-7.42 (m, 3H), 7.41-7.31 (m, 2H), 7.27 (d, J=1.9 Hz, 1H), 5.44 (s, 1H), 3.74 (s, 2H), 3.12-2.97 (m, 2H), 2.83-2.58 (m, 2H), 2.19 (td, J=13.3, 4.4 Hz, 1H), 2.03-1.78 (m, 3H). ESI-MS m z: 423.05 [M+1]⁺.

The second eluting peak was isolated to provide compound 194 5-chloro-1′-[(1-phenylpyrazol-4-yl)methyl]spiro[1H-isobenzofuran-3,4′-piperidine]-1-carboxamide (6.2 mg, 78%) ¹H NMR (300 MHz, Chloroform-d) δ 8.26 (s, 1H), 7.79-7.70 (m, 3H), 7.54-7.30 (m, 5H), 7.27 (d, J=1.9 Hz, 1H), 5.44 (s, 1H), 3.74 (s, 2H), 3.04 (t, J=11.3 Hz, 2H), 2.80-2.55 (m, 2H), 2.19 (td, J=13.3, 4.5 Hz, 1H), 2.04-1.74 (m, 3H). ESI-MS m/z: 423.05 [M+1]⁺.

The aqueous layer from the above reaction mixture was concentrated under reduced pressure to give a crude residue that was purified by reverse phase HPLC (Gradient: 45-99% MeCN in 0.1% aqueous formic acid) to provide 5-chloro-1′-[(1-phenylpyrazol-4-yl)methyl]spiro[1H-isobenzofuran-3,4′-piperidine]-1-carboxylic acid 192 (20 mg, 10%) ¹H NMR (400 MHz, DMSO-d6) δ: 8.43 (s, 1H), 7.84 (d, J=8.4 Hz, 2H), 7.68 (s, 1H), 7.48 (t, J=8.4 Hz, 2H), 7.40-7.34 (m, 3H), 7.30-7.26 (m, 1H), 6.11 (brs, 1H), 5.52 (s, 1H), 3.57 (s, 2H), 2.82-2.81 (m, 2H), 2.49-2.32 (m, 2H), 2.07-2.00 (m, 2H), 1.98-1.90 (m, 1H), 1.62-1.55 (m, 1H). ESI-MS m/z: 424.03 [M+1]⁺.

### Compound 195

### 5-chloro-N-methyl-1′-[(1-phenylpyrazol-4-yl)methyl]spiro[1H-isobenzofuran-3,4′-piperidine]-1-carboxamide (195)

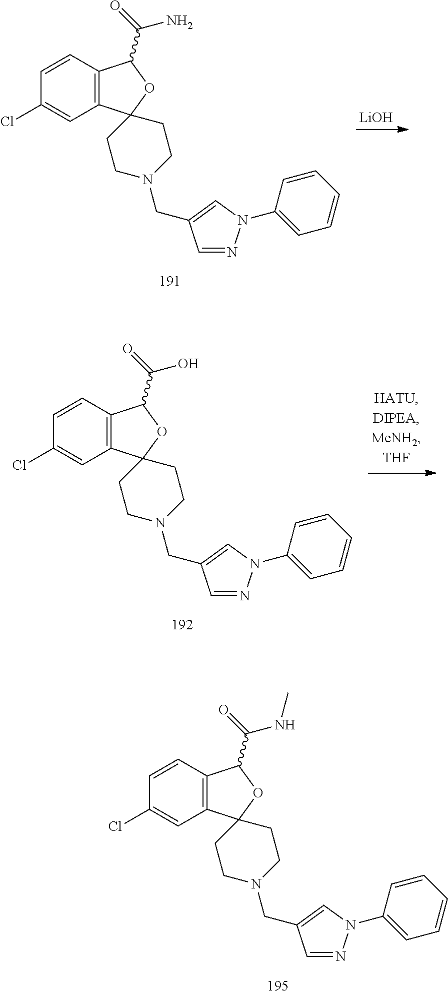

### Step 1. Synthesis of 5-chloro-1′-[(1-phenylpyrazol-4-yl)methyl]spiro[1H-isobenzofuran-3,4′-piperidine]-1-carboxylic acid (192)

To a stirred solution of 5-chloro-1′-[(1-phenylpyrazol-4-yl)methyl]spiro[1H-isobenzofuran-3,4′-piperidine]-1-carboxamide (400 mg, 0.8513 mmol) in THF (5 mL) was added LiOH—H₂O (40 mg, 0.0016 mol) in Water (5 mL) at room temperature and heated to 60° C. for 16 h. The reaction cooled to room temperature, acidified with 1 M HCl, and extracted with EtOAc (3×20 mL). The combined organic layers were dried over anhydrous sodium sulfate and concentrated under reduced pressure to afford crude 5-chloro-1′-[(1-phenylpyrazol-4-yl)methyl]spiro[1H-isobenzofuran-3,4′-piperidine]-1-carboxylic acid (180 mg, 44%) as a yellow gum which was used in the subsequent step without further purification. LCMS m/z 423.96 [M+1]⁺.

### Step 2. Synthesis of 5-chloro-N-methyl-1′-[(1-phenylpyrazol-4-yl)methyl]spiro[1H-isobenzofuran-3,4′-piperidine]-1-carboxamide (195)

To a stirred solution of 5-chloro-1′-[(1-phenylpyrazol-4-yl)methyl]spiro[1H-isobenzofuran-3,4′-piperidine]-1-carboxylic acid (110 mg, 207.31 mol) in THF (2.0 mL) was added HATU (320 mg, 833.18 μmol), DIPEA (146.92 mg, 0.2 mL, 0.0011 mol) and Methylamine (Hydrochloric Acid (1)) (40 mg, 586.51 μmol) at room temperature. The reaction mixture was stirred for 16 h at room temperature. The reaction was quenched with water (50 mL) and extracted with 10% methanol in DCM (2×100 mL). The organic layer was dried over sodium sulfate and concentrated under reduced pressure. The resulting residue was purified by reverse phase chromatography (Column: x-select phenyl hexyl. Gradient: 0-100% MeCN in water with 0.1% formic acid) which after lyophilization afforded 5-chloro-N-methyl-1′-[(1-phenylpyrazol-4-yl)methyl]spiro[1H-isobenzofuran-3,4′-piperidine]-1-carboxamide (Formic Acid (1)) 195 (50 mg, 48%) as a white solid. ¹H NMR (400 MHz, DMSO-d6): δ 8.42 (s, 1H), 8.31 (s, 1H), 7.83 (d, J=8.8 Hz, 2H), 7.67 (s, 1H), 7.62-7.59 (m, 1H), 7.48 (t, J=8.4 Hz, 2H), 7.41-7.32 (m, 3H), 7.28 (t, J=7.2 Hz, 1H), 5.39 (s, 1H), 3.55 (s, 2H), 2.86-2.75 (m, 2H), 2.62 (d, J=4.8 Hz, 3H), 2.49-2.45 (m, 1H), 2.36-2.31 (m, 1H), 2.12-2.05 (m, 1H), 1.90-1.78 (m, 2H), 1.65-1.60 (m, 1H). LCMS m/z 435.19 [M−1]⁻.

### Compounds 196-202

Compounds 196-202 (see Table 15) were prepared from the appropriate nitrile intermediates employing the same method as compound 191 or of compound 191 then the method of compound 192. All nitriles were synthesized as described above. Any modifications to methods are noted in Table 15 and accompanying footnotes.

TABLE 15
Structure and physicochemical data for Compounds 196-202

| | | | | ¹H NMR; |
|---|---|---|---|---|
| | | | Me- | LCMS m/z |
| Cmpd | Product | Nitrile | thod | [M + H]⁺ |
| 196 | 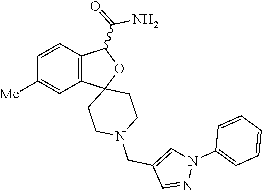 [SMILES: Cc1ccc2c(c1)C1(CCN(Cc3cnn(-c4ccccc4)c3)CC1)OC2C(N)=O] | 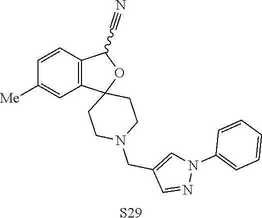 [SMILES: Cc1ccc2c(c1)C1(CCN(Cc3cnn(-c4ccccc4)c3)CC1)OC2C#N ; "S29"] | Cmpd 191 | ¹H NMR (400 MHz, DMSO- d6): δ 8.42 (s, 1H), 7.83 (d, J = 8 Hz, 2H), 7.67 (s, 1H), 7.48 (t, J = 7.6 Hz, 2H), 7.33-7.24 (m, 3H), 7.10-7.01 (m, 3H), 5.29 (s, 1H), 3.51 (s, 2H), 2.80 (brs, 2H), 2.50-2.31 |
| | | | | (m, 5H), 2.05- |
| | | | | 1.97 (m, 1H), |
| | | | | 1.86-1.79 (m, |
| | | | | 2H), 1.62-1.58 |
| | | | | (m, 1H). LCMS |
| | | | | m/z 403.2 |
| | | | | [M + 1]⁺. |
| 197 | 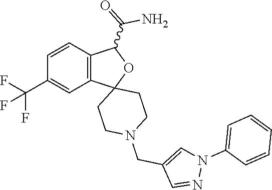 [SMILES: NC(=O)C1OC2(CCN(Cc3cnn(-c4ccccc4)c3)CC2)c2cc(C(F)(F)F)ccc21] | 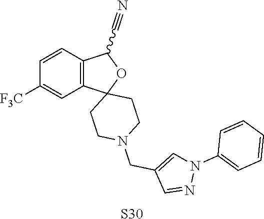 [SMILES: N#CC1OC2(CCN(Cc3cnn(-c4ccccc4)c3)CC2)c2cc(C(F)(F)F)ccc21 ; "S30"] | Cmpd 191 | ¹H NMR (400 MHz, DMSO- d6): δ 8.41 (s, 1H), 7.83 (d, J = 8.4 Hz, 2H), 7.71-7.68 (m, 3H), 7.60 (d, J = 8.0, 1H), 7.50- 7.46 (m, 3H), 7.28 (t, J = 7.6 Hz, 1H), 7.19 (s, 1H), 5.47 (s, 1H), 3.56 (s, 2H), 2.83-2.81 |
| | | | | (m, 2H), 2.54 (s, |
| | | | | 1H), 2.34 (t, J = |
| | | | | 9.6 Hz, 1H), |
| | | | | 2.18-2.11 (m, |
| | | | | 1H), 1.98-1.92 |
| | | | | (m, 2H), 1.68 (d, |
| | | | | J = 12.0 Hz, |
| | | | | 1H). LCMS m/z |
| | | | | 457.04 [M + 1]⁺. |
| 198 | 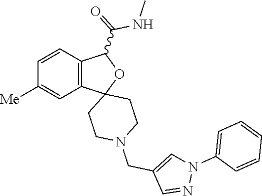 [SMILES: CNC(=O)C1OC2(CCN(Cc3cnn(-c4ccccc4)c3)CC2)c2cc(C)ccc21] | 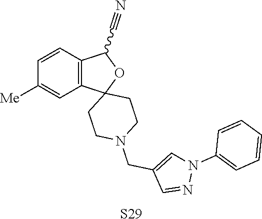 [SMILES: Cc1ccc2c(c1)C1(CCN(Cc3cnn(-c4ccccc4)c3)CC1)OC2C#N ; "S29"] | Cmpd 191 then Cmpd 195 | ¹H NMR (400 MHz, DMSO- d6) δ: (s, 1H), 7.83 (d, J = 8.0 Hz, 2H), 7.68 (s, 1H), 7.50-7.47(m, 3H), 7.30-7.23 (m, 2H), 7.09- 7.05 (m, 2H), 5.33 (s, 1H), 3.53 (s, 2H), 2.81 (brs, 2H), 2.67-2.50 (m, |
| | | | | 4H), 2.30 (s, |
| | | | | 4H), 2.07-2.00 |
| | | | | (m, 1H), 1.84- |
| | | | | 1.77 (m, 2H), |
| | | | | 1.62-1.59 (m, |
| | | | | 1H). LCMS m/z |
| | | | | 417.11 [M + 1]⁺. |
| 199 | 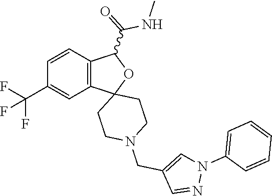 [SMILES: CNC(=O)C1OC2(CCN(Cc3cnn(-c4ccccc4)c3)CC2)c2cc(C(F)(F)F)ccc21] | 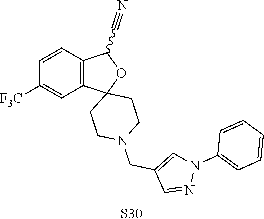 [SMILES: N#CC1OC2(CCN(Cc3cnn(-c4ccccc4)c3)CC2)c2cc(C(F)(F)F)ccc21 ; "S30"] | Cmpd 191 then Cmpd 195¹ | ¹H NMR (400 MHz, DMSO- d6): δ 8.42 (s, 1H), 8.19 (s, 1H), 7.83 (d, J = 8.4 Hz, 2H), 7.71-7.66 (m, 4H), 7.58 (d, J = 8.0, 1H), 7.50 (d, J = 7.6 Hz, 2H), 7.28 (t, J = 8.0 Hz, 1H), 5.51 (s, 1H), 5.57 (s, 2H), |
| | | | | 2.82 (br s, 2H), |
| | | | | 2.63 (d, J = 4.8 |
| | | | | Hz, 1H), 2.56- |
| | | | | 2.54 (m, 1H), |
| | | | | 2.36 (t, J = 11.6 |
| | | | | Hz, 1H), 2.19- |
| | | | | 2.14 (m, 1H), |
| | | | | 1.94-1.84 (m, |
| | | | | 2H), 1.67 (d, J = |
| | | | | 13.0 Hz, 1H), |
| | | | | 1.23 (s, |
| | | | | 1H). LCMS |
| | | | | m/z 471.21 |
| | | | | [M + 1]⁺. |
| 200 | 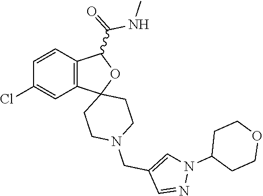 [SMILES: CNC(=O)C1OC2(CCN(Cc3cnn(C4CCOCC4)c3)CC2)c2cc(Cl)ccc21] | 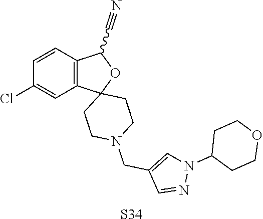 [SMILES: N#CC1OC2(CCN(Cc3cnn(C4CCOCC4)c3)CC2)c2cc(Cl)ccc21 ; "S34"] | Cmpd 191 then Cmpd 195¹ | ¹H NMR (400 MHz DMSO-d6): δ 7.68 (s, 1H), 7.59 (d, J = 4.4 Hz, 1H), 7.04 (brs, 1H), 7.38- 7.32 (m, 3H), 5.39 (s, 1H), 4.35-3.95 (m, 1H), 3.93-3.92 (m, 2H), 3.47- 3.31 (m, 4H), 2.75(brs, 2H), |
| | | | | 2.62(d, J = 4.8 |
| | | | | Hz, 3H), 2.49- |
| | | | | 2.43 (m, 1H), |
| | | | | 2.26 (brs, 1H), |
| | | | | 2.07-1.90 (m, |
| | | | | 1H), 1.89-1.78 |
| | | | | (m, 6H), 1.61 |
| | | | | (d, J = 11.2 Hz, |
| | | | | 1H). LCMS m/z |
| | | | | 445.1 [M + 1]⁺. |
| 201 | 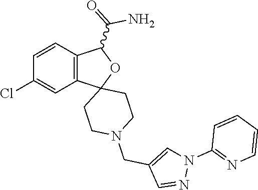 [SMILES: NC(=O)C1OC2(CCN(Cc3cnn(-c4ccccn4)c3)CC2)c2cc(Cl)ccc21] | 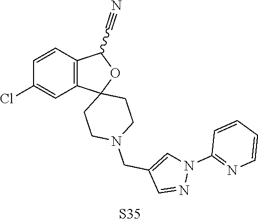 [SMILES: N#CC1OC2(CCN(Cc3cnn(-c4ccccn4)c3)CC2)c2cc(Cl)ccc21 ; "S35"] | Cmpd 191 | ¹H NMR (400 MHz, DMSO-d6) δ 8.51 (s, 1H), 8.46 (dd, J1 = 1.2 Hz, J2 = 5.2 Hz, 1H), 8.00- 7.96 (m, 1H), 7.90 (d, J = 8.4 Hz, 1H), 7.75 (s, 1H), 7.43- 7.41 (m, 2H), 7.39-7.32 (m, 3H), 7.11 (brs, |
| | | | | 1H), 5.34 (s, |
| | | | | 1H), 3.55 (s, |
| | | | | 2H), 2.79 (d, J = |
| | | | | 10.4 Hz, 2H), |
| | | | | 2.51-2.46 (m, |
| | | | | 1H), 2.35 (t, J = |
| | | | | 10.0 Hz, 1H), |
| | | | | 2.10-2.03 (m, |
| | | | | 1H), 1.92-1.88 |
| | | | | (m, 1H), 1.86- |
| | | | | 1.79 (m, 1H), |
| | | | | 1.62 (d, J = |
| | | | | 11.2 Hz, |
| | | | | 1H). LCMS |
| | | | | m/z 424.1 |
| | | | | [M + 1]⁺. |
| 202 | 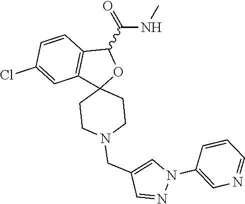 [SMILES: CNC(=O)C1OC2(CCN(Cc3cnn(-c4cccnc4)c3)CC2)c2cc(Cl)ccc21] | 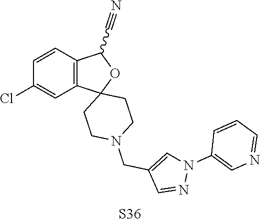 [SMILES: N#CC1OC2(CCN(Cc3cnn(-c4cccnc4)c3)CC2)c2cc(Cl)ccc21 ; "S36"] | Cmpd 191 then Cmpd 195¹ | ¹H NMR (400 MHz, DMSO-d6) δ: 9.10 (d, J = 2.4 Hz, 1H), 8.53 (s, 1H), 8.50(dd, J = 4.8 Hz, 1.2 Hz, 1H), 8.24- 8.21 (m, 1H), 7.75 (s, 1H), 7.60 (d, J = 4.8 Hz, 1H), 7.55- 7.52 (s, 1H), 7.40 (s, 1H), |
| | | | | 7.38-7.35 (m, |
| | | | | 2H), 5.40 (s, |
| | | | | 1H), 3.56 (s, |
| | | | | 2H), 2.80 (brs, |
| | | | | 2H), 2.67 (s, |
| | | | | 3H), 2.66- |
| | | | | 2.34(m, 1H), |
| | | | | 2.37-2.31(m, |
| | | | | 1H), 2.12- |
| | | | | 2.04(m, 1H), |
| | | | | 1.88-1.85 (m, |
| | | | | 2H), 1.63-1.60 |
| | | | | (m, 1H). LCMS |
| | | | | m/z 438.37 |
| | | | | [M + 1]⁺. |

¹T3P and Et₃N used instead of HATU and DIPEA for amide coupling

### Compound 203

### 5-cyano-1′-[(1-phenylpyrazol-4-yl)methyl]spiro[1H-isobenzofuran-3,4′-piperidine]-1-carboxamide (203)

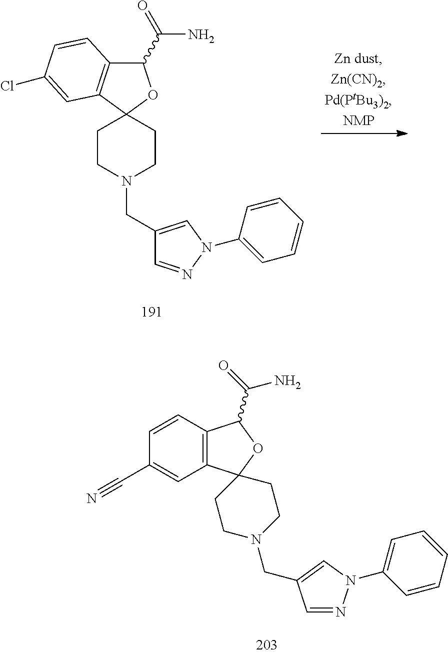 [SMILES: NC(=O)C1OC2(CCN(Cc3cnn(-c4ccccc4)c3)CC2)c2cc(Cl)ccc21 ; N#Cc1ccc2c(c1)C1(CCN(Cc3cnn(-c4ccccc4)c3)CC1)OC2C(N)=O]

To a stirred solution of 5-chloro-1′-[(1-phenylpyrazol-4-yl)methyl]spiro[1H-isobenzofuran-3,4′-piperidine]-1-carboxamide (191) (100 mg, 0.2190 mmol) in NMP (3 mL) was added Zn(CN)₂ (60 mg, 500.79 μmol) and Zn dust (700 mg, 0.0981 mL, 10.491 mmol). The reaction mixture was degassed with argon for 10 minutes. Then added Bis(tri-tert-butylphosphine)palladium(0) (75 mg, 143.82 μmol) was added and the solution was again degassed for 5 minutes at room temperature. The reaction mixture was irradiated under microwave condition at 150° C. for 1 h. The reaction mixture was diluted with water (10 mL) and extracted with EtOAC (30 2×mL) and dried over anhydrous sodium sulfate, and concentrated under reduced pressure. The crude residue was blended with an additional reaction of the same conditions starting with 50 mg of 5-chloro-1′-[(1-phenylpyrazol-4-yl)methyl]spiro[1H-isobenzofuran-3,4′-piperidine]-1-carboxamide (2), and then purified by reverse phase chromatography (Column:C18. Gradient: 0-100% MeCN in aqueous solution of 10 mM Ammonium Bicarbonate) which after lyophilization afforded 5-cyano-1′-[(1-phenylpyrazol-4-yl)methyl]spiro[1H-isobenzofuran-3,4′-piperidine]-1-carboxamide (17 mg, 12.5% yield) as a white solid. ¹H NMR (400 MHz, DMSO-d6) δ: 8.41 (s, 1H), 7.87 (s, 1H), 7.83 (dd, J=1.2, 8.8 Hz, 2H), 7.77 (dd, J=1.2, 7.6 Hz, 1H), 7.67 (s, 1H), 7.57 (d, J=7.6 Hz, 1H), 7.48 (dd, J=2, 7.2 H, 3H), 7.28 (t, J=7.6 Hz, 1H), 7.21 (s, 1H), 5.47 (s, 1H), 3.52 (s, 2H), 2.81 (d, J=8 Hz, 2H), 2.51-2.46 (m, 1H), 2.35 (t, J=10 Hz, 1H), 2.12-2.06 (m, 1H), 1.93-1.88 (m, 2H), 1.60-1.50 (m, 1H). LCMS m/z 414.1 [M+1]⁺.

### Compound 204

### 5-cyano-N-methyl-1′-[(1-phenylpyrazol-4-yl)methyl]spiro[1H-isobenzofuran-3,4′-piperidine]-1-carboxamide (204)

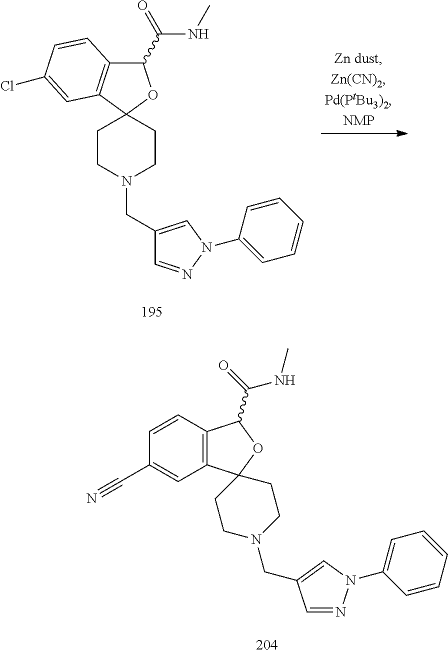 [SMILES: CNC(=O)C1OC2(CCN(Cc3cnn(-c4ccccc4)c3)CC2)c2cc(Cl)ccc21 ; CNC(=O)C1OC2(CCN(Cc3cnn(-c4ccccc4)c3)CC2)c2cc(C#N)ccc21]

5-cyano-N-methyl-1′-[(1-phenylpyrazol-4-yl)methyl]spiro[1H-isobenzofuran-3,4′-piperidine]-1-carboxamide (204) was prepared using the same method as used to prepare compound 203 with with the appropriate starting aryl chloride.

¹H NMR (400 MHz, DMSO-d6) δ: 8.42 (s, 1H), 7.87-7.82 (m, 3H), 7.76 (dd, J=1.2, 8 Hz, 1H), 7.68 (d, J=6.4 Hz, 2H), 7.56 (d, J=8 Hz, 1H), 7.49 (t, J=7.6 Hz, 2H), 7.28 (t, J=7.6 Hz, 1H), 5.51 (s, 1H), 3.42 (s, 2H), 2.81 (brs, 2H), 2.62 (d, J=4.8 Hz, 3H), 2.54-2.49 (m, 1H), 2.37-2.31 (m, 1H), 2.15-2.11 (m, 1H), 1.91-1.82 (m, 2H), 1.65 (d, J=11.2 Hz, 1H) LCMS m/z 428.46 [M+1]⁺.

### Preparation of S37

### 1′-[(1-phenylpyrazol-4-yl)methyl]-5-vinyl-spiro[1H-isobenzofuran-3,4′-piperidine]-1-carbonitrile (S37)

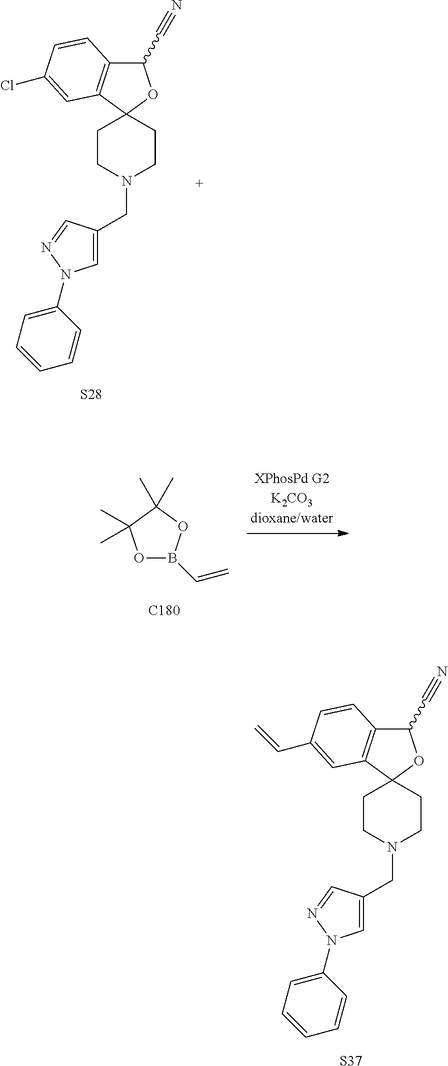 [SMILES: N#CC1OC2(CCN(Cc3cnn(-c4ccccc4)c3)CC2)c2cc(Cl)ccc21 ; "S28" ; C=CB1OC(C)(C)C(C)(C)O1 ; "C180" ; C=Cc1ccc2c(c1)C1(CCN(Cc3cnn(-c4ccccc4)c3)CC1)OC2C#N ; "S37"]

To a stirred solution of 5-chloro-1′-[(1-phenylpyrazol-4-yl)methyl]spiro[1H-isobenzofuran-3,4′-piperidine]-1-carbonitrile (500 mg, 0.0011 mol) in Dioxane (25 mL) and Water (7 mL) were added 4,4,5,5-tetramethyl-2-vinyl-1,3,2-dioxaborolane (240 mg, 0.0015 mol), and K₂CO₃ (610 mg, 0.0043 mol). The reaction mixture was degassed with argon for 10 minutes. Then added XPhosPd-G2 (160 mg, 199.03 μmol) again degassed for 5 minutes at room temperature in a sealed tube. The reaction mixture was stirred at 100° C. for 16 h. The reaction mixture was diluted with water (50 mL) and extracted with EtOAC (2×50 mL), dried over anhydrous sodium sulfate, and concentrated under reduced pressure. The resulting crude residue was purified by silica gel column chromatography silica gel chromatography (Gradient: 0-50% EtOAc in Pet ether) to afford 1′-[(1-phenylpyrazol-4-yl)methyl]-5-vinyl-spiro[1H-isobenzofuran-3,4′-piperidine]-1-carbonitrile (350 mg, 80% yield), as a yellow solid. ¹H NMR (400 MHz, DMSO-d6) δ: 8.43 (s, 1H), 7.83 (d, J=7.6 Hz, 2H), 7.68-7.47 (m, 6H), 7.29 (t, J=6.8 Hz, 1H), 6.78 (dd, J=11.2, 18 Hz, 1H), 6.20 (s, 1H), 5.95 (d, J=17.6 Hz, 1H), 5.33 (d, J=10.8 Hz, 1H), 3.52 (s, 2H), 2.85 (br s, 2H), 2.32 (br s, 2H), 2.13-1.98 (m, 2H), 1.76 (d, J=12.4 Hz, 1H), 1.63 (d, J=12.4 Hz, 1H). LCMS m/z 397.19 [M+1]⁺.

### Preparation of C181 and 182

Intermediate compounds C181 and C182 (see Table 16) were prepared from the nitrile intermediate S37 using either the method used to prepare compound 191 or the method used to prepare compound 195. Any modifications to methods are noted in Table 16 and accompanying footnotes.

TABLE 16
Structure and physicochemical data for compounds C181 and C182

| | | | | LCMS m/z |
|---|---|---|---|---|
| Cmpd | Product | Nitrile | Method | [M + H]⁺ |
| C181 | 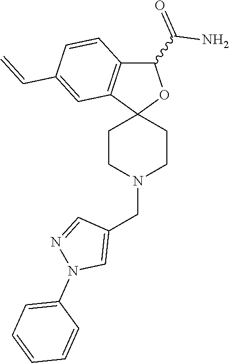 [SMILES: C=Cc1ccc2c(c1)C1(CCN(Cc3cnn(-c4ccccc4)c3)CC1)OC2C(N)=O] | 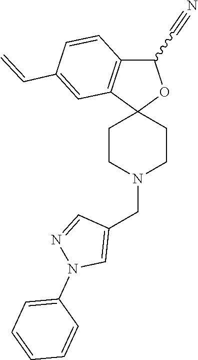 | Compound 191 | LCMS m/z 415.21 [M + 1]⁺ |
| | | S37 | | |
| C182 | 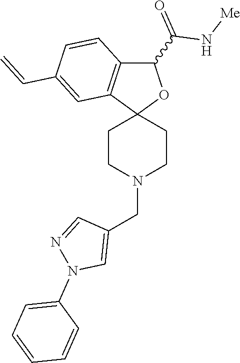 [SMILES: C=Cc1ccc2c(c1)C1(CCN(Cc3cnn(-c4ccccc4)c3)CC1)OC2C(=O)NC] | 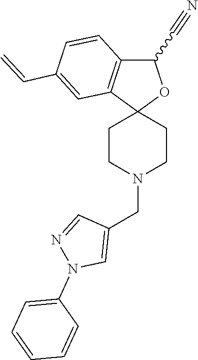 | Compound 191 then Compound 195¹ | LCMS m/z 429.52 [M + 1]⁺ |
| | | S37 | | |

¹T3P used instead of HATU for amide coupling

### Compound 205

### 5-ethyl-1′-[(1-phenylpyrazol-4-yl)methyl]spiro[1H-isobenzofuran-3,4′-piperidine]-1-carboxamide (205)

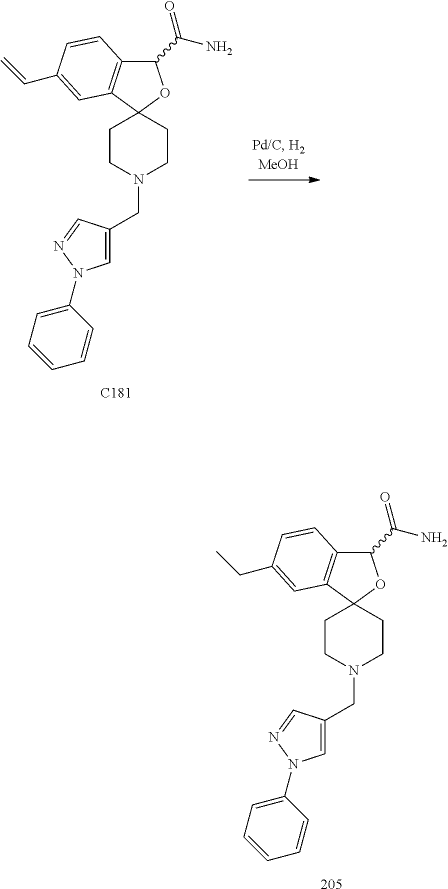

To a stirred solution of 1′-[(1-phenylpyrazol-4-yl)methyl]-5-vinyl-spiro[1H-isobenzofuran-3,4′-piperidine]-1-carboxamide (40 mg, 79.131 mol) in MeOH (5 mL) was added Palladium on carbon (40 mg, 10% w/w, 0.0376 mmol). The reaction mass was stirred under hydrogen balloon pressure at room temperature for 16 h. The reaction was filtered through a pad of celite and washed with MeOH (25 mL), and evaporated under vacuum. The crude residue was purified by reverse phase chromatography (Column:C18. Gradient: 0-100% MeCN in aqueous solution of 0.1% TFA) which after lyophilization afforded 5-ethyl-1′-[(1-phenylpyrazol-4-yl)methyl]spiro[1H-isobenzofuran-3,4′-piperidine]-1-carboxamide (Trifluoroacetic Acid (1)) 205 (10 mg, 23% yield) as an off white sticky solid. ¹H NMR (400 MHz, DMSO-d6) δ: 10.6 (brs, 1H), 8.60 (br s, 1H), 7.85-7.83 (m, 3H), 7.53 (t, J=7.6 Hz, 2H), 7.37-7.17 (m, 5H), 6.99 (br s, 1H), 5.38 (s, 1H), 4.19-4.15 (m, 2H), 3.32-2.66 (m, 4H), 2.62 (t, J=7.6 Hz, 2H), 2.21 (br s, 1H), 1.90 (br s, 2H), 1.80 (d, J=9.2 Hz, 1H), 1.24 (s, 3H), 19F NMR (376.75 MHz, DMSO-d6) δ: −73.49. LCMS m/z 417.28 [M+1]⁺.

### Compound 206

### 5-ethyl-N-methyl-1′-[(1-phenylpyrazol-4-yl)methyl]spiro[1H-isobenzofuran-3,4′-piperidine]-1-carboxamide (206)

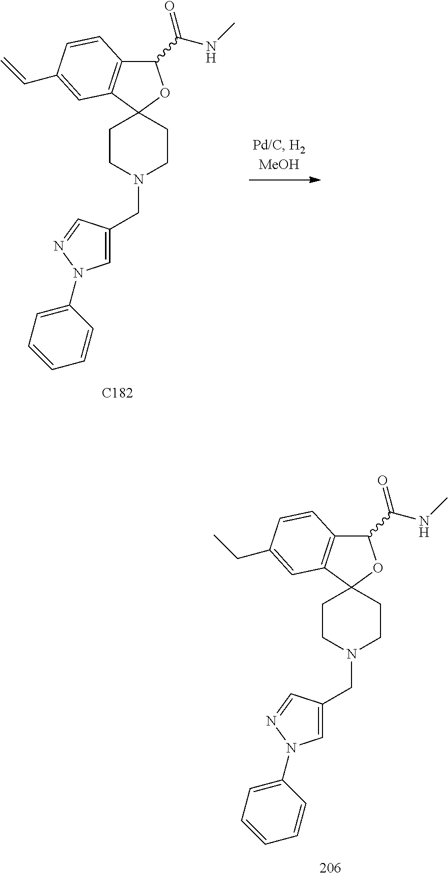

5-ethyl-N-methyl-1′-[(1-phenylpyrazol-4-yl)methyl]spiro[1H-isobenzofuran-3,4′-piperidine]-1-carboxamide (206) was prepared using the same method as used to prepare Compound 205 with starting methyl amide C182.

¹H NMR (400 MHz, DMSO-d6) δ: 8.40 (s, 1H), 7.82 (d, J=8 Hz, 2H), 7.67 (s, 1H), 7.48 (t, J=7.6 Hz, 3H), 7.30-7.24 (m, 2H), 7.10 (t, J=8 Hz, 2H), 5.34 (s, 1H), 3.52 (s, 2H), 2.80 (t, J=10.8 Hz, 2H), 2.63-2.53 (m, 5H), 2.51-2.49 (m, 1H), 2.34 (t, J=11.2 Hz, 1H), 2.07-2.04 (m, 1H), 1.85-1.78 (m, 2H), 1.63 (s, 1H), 1.16 (t, J=7.6 Hz, 3H). LCMS m/z 431.33 [M+1]⁺.

### Compound 207

### 1′-[[1-(2-methylsulfonylethyl)triazol-4-yl]methyl]-5-(trifluoromethyl)spiro[1H-isobenzofuran-3,4′-piperidine]-1-carboxamide (207)

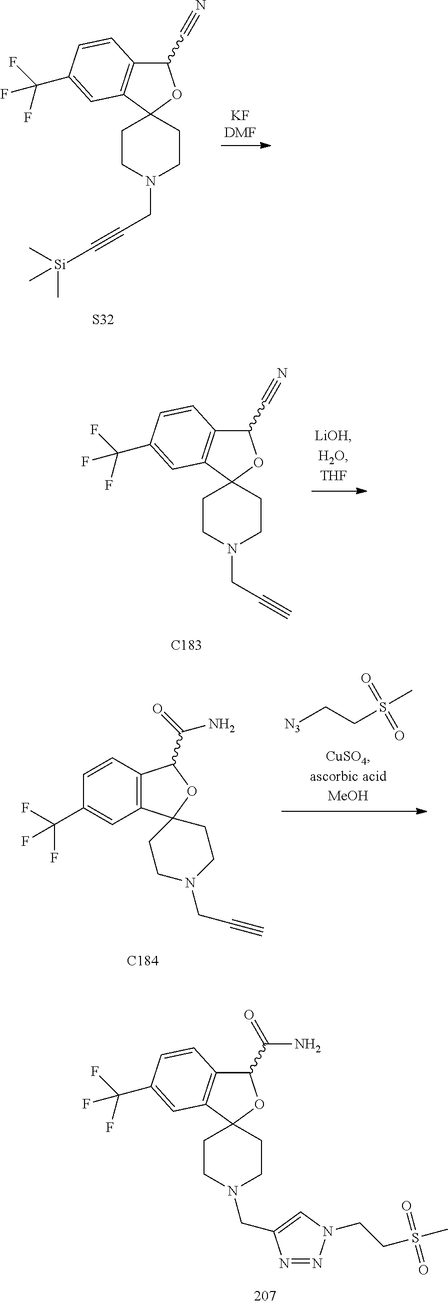

### Step 1. Synthesis of 1′-prop-2-ynyl-5-(trifluoromethyl)spiro[1H-isobenzofuran-3,4′-piperidine]-1-carbonitrile (C183)

To a stirred solution of 5-(trifluoromethyl)-1′-(3-trimethylsilylprop-2-ynyl)spiro[1H-isobenzofuran-3,4′-piperidine]-1-carbonitrile (1 g, 0.0025 mol) in DMF (10 mL) was added KF (5 g, 0.0843 mol) at 0° C. and stirred for 6 hours at same temperature. The reaction was quenched with saturated NH₄Cl solution (50 mL) and extracted with ethyl acetate (2×100 mL). The organic layer was dried over sodium sulfate and concentrated under reduced pressure. The crude residue was purified by silica gel chromatography (Isocratic: 10% EtOAc in hexane) to afford 1′-prop-2-ynyl-5-(trifluoromethyl)spiro[1H-isobenzofuran-3,4′-piperidine]-1-carbonitrile (715 mg, 86% yield) as an off white sticky solid. ¹H NMR (DMSO-d6, 400 MHz): δ 7.91 (s, 1H), 7.83-7.77 (m, 2H), 6.36 (s, 1H), 3.35 (d, J=2.4 Hz, 2H), 3.18 (t, J=2.4 Hz, 1H), 2.79-2.74 (m, 2H), 2.57-2.52 (m, 2H), 2.17-2.08 (m, 2H), 1.80-1.75 (m, 1H), 1.68-1.64 (m, 1H). L CMS m/z 321.11 [M+1]⁺.

### Step 2. Synthesis of 1′-prop-2-ynyl-5-(trifluoromethyl)spiro[1H-isobenzofuran-3,4′-piperidine]-1-carboxamide (C184)

To a solution of 1′-prop-2-ynyl-5-(trifluoromethyl)spiro[1H-isobenzofuran-3,4′-piperidine]-1-carbonitrile (32 mg, 0.09990 mmol) in THF (0.5 mL) was added LiOH (12 mg, 0.5011 mmol) and water (0.5 mL). The mixture was stirred at room temperature overnight. The mixture was extracted with DCM (2×2 mL), and the organic layer was concentrated in vacuo to afford 1′-prop-2-ynyl-5-(trifluoromethyl)spiro[1H-isobenzofuran-3,4′-piperidine]-1-carboxamide which was used in subsequent steps without further purification. LCMS m/z 339.19 [M+1]⁺.

### Step 3. Synthesis of 1′-[[1-(2-methylsulfonylethyl)triazol-4-yl]methyl]-5-(trifluoromethyl)spiro[1H-isobenzofuran-3,4′-piperidine]-1-carboxamide (207)

The crude 1′-prop-2-ynyl-5-(trifluoromethyl)spiro[1H-isobenzofuran-3,4′-piperidine]-1-carboxamide was dissolved in MeOH (0.5 mL) and a solution of 1-azido-2-methylsulfonyl-ethane (15 mg, 0.1006 mmol) in MeOH (0.5 mL) containing copper sulfate (0.08 mL of 1% w/v, 0.005012 mmol) was added. Then, ascorbic acid (Sodium salt) (2 mg, 0.01004 mmol) was added, and the mixture was stirred at room temperature for 1 h. The residue was concentrated in vacuo and purification by reversed-phase HPLC. Method: C18 Waters Sunfire column (30×150 mm, 5 micron). Gradient: MeCN in H₂O with 0.1% trifluoroacetic acid to afford 1′-[[1-(2-methylsulfonylethyl)triazol-4-yl]methyl]-5-(trifluoromethyl)spiro[1H-isobenzofuran-3,4′-piperidine]-1-carboxamide (Trifluoroacetate salt) (14.8 mg, 24% yield over 2 steps) as a pale yellow sticky solid. ¹H NMR (400 MHz, DMSO-d6) δ 8.41 (d, J=17.3 Hz, 1H), 7.78 (d, J=8.0 Hz, 1H), 7.67 (d, J=8.1 Hz, 1H), 7.54 (s, 2H), 7.47 (s, 1H), 5.58 (s, 1H), 4.92 (t, J=6.6 Hz, 2H), 4.57 (s, 2H), 3.85 (t, J=6.7 Hz, 2H), 3.73 (t, J=7.2 Hz, 1H), 3.28 (d, J=12.1 Hz, 1H), 3.02 (s, 3H), 2.31 (d, J=15.3 Hz, 1H), 2.13 (d, J=14.5 Hz, 2H), 1.96 (d, J=14.4 Hz, 1H). LCMS m/z 488.34 [M+1]⁺.

### Compound 208

### 5-chloro-1′-[[1-(2-methylsulfonylethyl)triazol-4-yl]methyl]spiro[1H-isobenzofuran-3,4′-piperidine]-1-carboxamide (208)

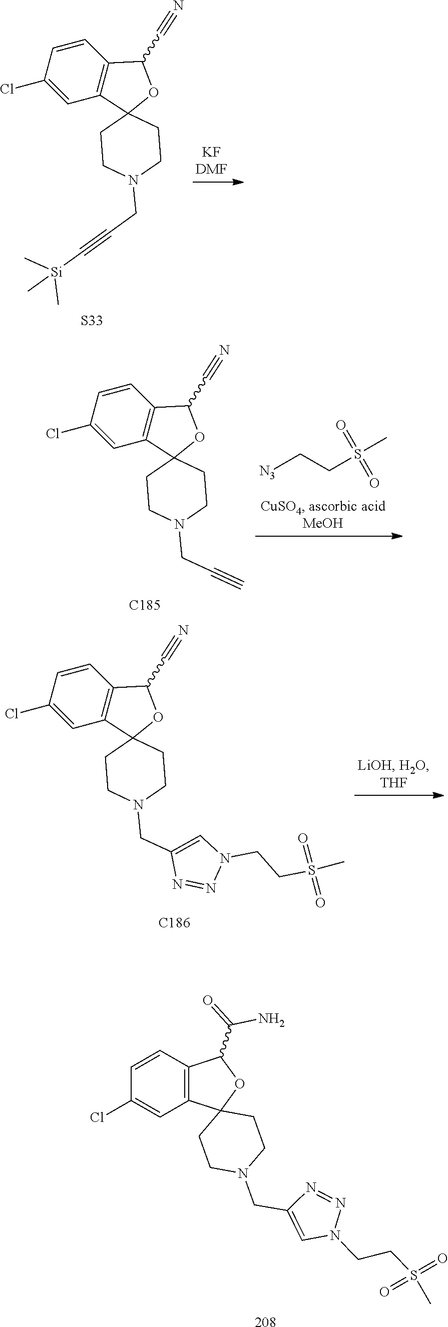

5-chloro-1′-[[1-(2-methylsulfonylethyl)triazol-4-yl]methyl]spiro[1H-isobenzofuran-3,4′-piperidine]-1-carboxamide (208) was prepared using the same method as used to prepare compound 207 with starting nitrile S33, with the modification that the LiOH hydrolysis step was performed after the copper sulfate catalyzed reaction in this sequence.

¹H NMR (300 MHz, MeOD) δ 8.32 (s, 1H), 7.51 (dt, J=8.1, 0.8 Hz, 1H), 7.40 (dd, J=8.2, 1.8 Hz, 1H), 7.28 (s, 1H), 5.53 (d, J=1.1 Hz, 1H), 5.08-4.96 (m, 2H), 4.58 (s, 2H), 3.91-3.73 (m, 2H), 3.61 (s, 3H), 3.46 (t, J=12.1 Hz, 1H), 3.00 (d, J=6.3 Hz, 3H), 2.32 (d, J=17.0 Hz, 1H), 2.22-1.99 (m, 3H). LCMS m/z 454.29 [M+1]⁺.

### Compound 209

### 5-chloro-1′-[[1-(2-methylpyrimidin-5-yl)pyrazol-4-yl]methyl]spiro[1H-isobenzofuran-3,4′-piperidine]-1-carboxamide (209)

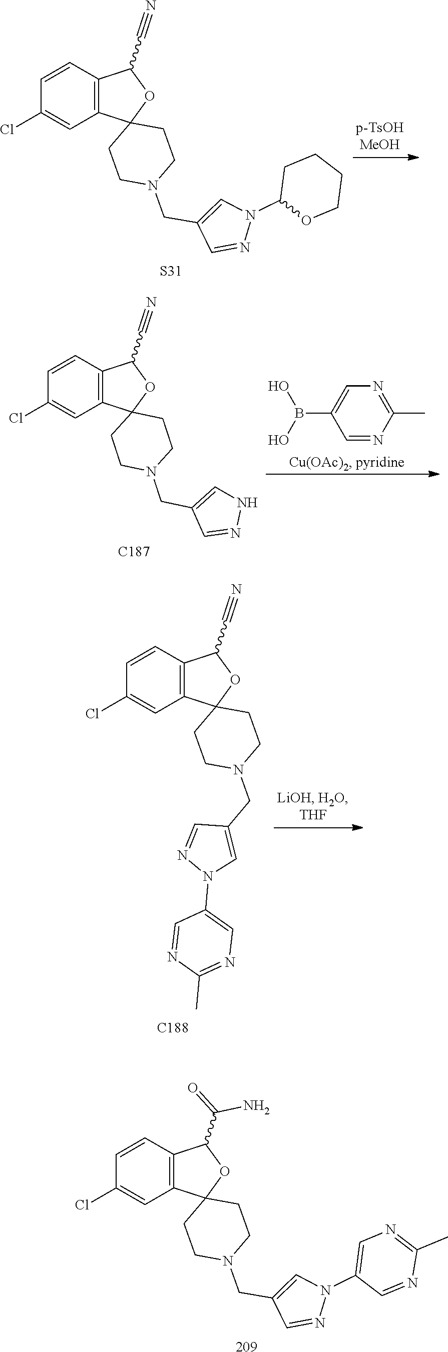

### Step 1. Synthesis of 5-chloro-1′-(H-pyrazol-4-ylmethyl)spiro[1H-isobenzofuran-3,4′-piperidine]-1-carbonitrile (C187)

To a stirred solution of 5-chloro-1′-[(1-tetrahydropyran-2-ylpyrazol-4-yl)methyl]spiro[1H-isobenzofuran-3,4′-piperidine]-1-carbonitrile (450 mg, 0.6892 mmol) in MeOH (6 mL) was added p-TsOH (180 mg, 0.1682 mL, 1.0244 mmol) at room temperature. The reaction mixture was stirred at room temperature for 16 h. At which point the reaction mixture was evaporated under reduced pressure. The crude residue was diluted in water (30 mL), brought to a pH ˜9 with 2 M NaOH (20 mL), extracted with extracted with EtOAc (2×30 mL), dried over anhydrous sodium sulfate, and concentrated under reduced pressure. The resulting crude residue was purified by silica gel column chromatography(Gradient: 0-60% Acetone/Petroleum ether) to afford an impure 5-chloro-1′-(1H-pyrazol-4-ylmethyl)spiro[1H-isobenzofuran-3,4′-piperidine]-1-carbonitrile (500 mg, 30% yield) as a sticky yellow gum. LCMS m/z 329.22 [M+1]⁺.

### Step 2. Synthesis of 5-chloro-1′-[[1-(2-methylpyrimidin-5-yl)pyrazol-4-yl]methyl]spiro[1H-isobenzofuran-3,4′-piperidine]-1-carbonitrile (C188)

To a stirred solution of 5-chloro-1′-(1H-pyrazol-4-ylmethyl)spiro[1H-isobenzofuran-3,4′-piperidine]-1-carbonitrile (300 mg, 0.6202 mmol) in THF (5.0 mL) was added (2-methylpyrimidin-5-yl)boronic acid (200 mg, 1.4355 mmol), Pyridine (297.00 mg, 0.3 mL, 3.7172 mmol), and Copper (II)acetate (11.375 mg, 0.0620 mmol) at room temperature. The reaction mixture was stirred under oxygen balloon at 60° C. for 16 h. The reaction mixture was diluted with ice cold water (50 mL), then extracted with EtOAc (3×100 mL). The organic layer was washed with brine (100 mL), dried over anhydrous sodium sulfate, filtered, and concentrated under reduced pressure to afford crude 5-chloro-1′-[[1-(2-methylpyrimidin-5-yl)pyrazol-4-yl]methyl]spiro[1H-isobenzofuran-3,4′-piperidine]-1-carbonitrile (400 mg, 31%) as a sticky solid which was used without further purification. LCMS m/z 420.96 [M+1]⁺.

### Step 3. Synthesis of 5-chloro-1′-[[1-(2-methylpyrimidin-5-yl)pyrazol-4-yl]methyl]spiro[1H-isobenzofuran-3,4′-piperidine]-1-carboxamide (209)

To a stirred solution of 5-chloro-1′-[[1-(2-methylpyrimidin-5-yl)pyrazol-4-yl]methyl]spiro[1H-isobenzofuran-3,4′-piperidine]-1-carbonitrile (30 mg, 45.011 μmol) in Water (1.0 mL) and THF (1.0 mL) was added LiOH·H₂O (4.0 mg, 94.368 μmol) at room temperature. The reaction mixture was stirred for 6 h. The reaction was concentrated under reduced pressure and the resulting residue was purified by silica gel column chromatography (Isocratic: 60% acetone in hexane) to afford 5-chloro-1′-[[1-(2-methylpyrimidin-5-yl)pyrazol-4-yl]methyl]spiro[1H-isobenzofuran-3,4′-piperidine]-1-carboxamide (9.0 mg, 41% yield) as an off white solid. ¹H NMR (400 MHz, DMSO-d6) δ: 9.17 (s, 2H), 8.55 (s, 1H), 7.79 (s, 1H), 7.43 (brs, 1H), 7.41-7.33 (m, 3H), 7.13 (brs, 1H), 5.35 (s, 1H), 3.52 (s, 2H), 2.81-2.79 (m, 2H), 2.69 (s, 3H), 2.49 (s, 1H), 2.37-2.32 (m, 1H), 2.11-2.02 (m, 1H), 1.92-1.83 (m, 2H), 1.63-1.60 (m, 1H). LCMS m/z 439.1 [M+1]⁺.

### Compound 210

### 5-chloro-N-methyl-1′-[[1-(2-methylsulfonylethyl)pyrazol-4-yl]methyl]spiro[1H-isobenzofuran-3,4′-piperidine]-1-carboxamide (210)

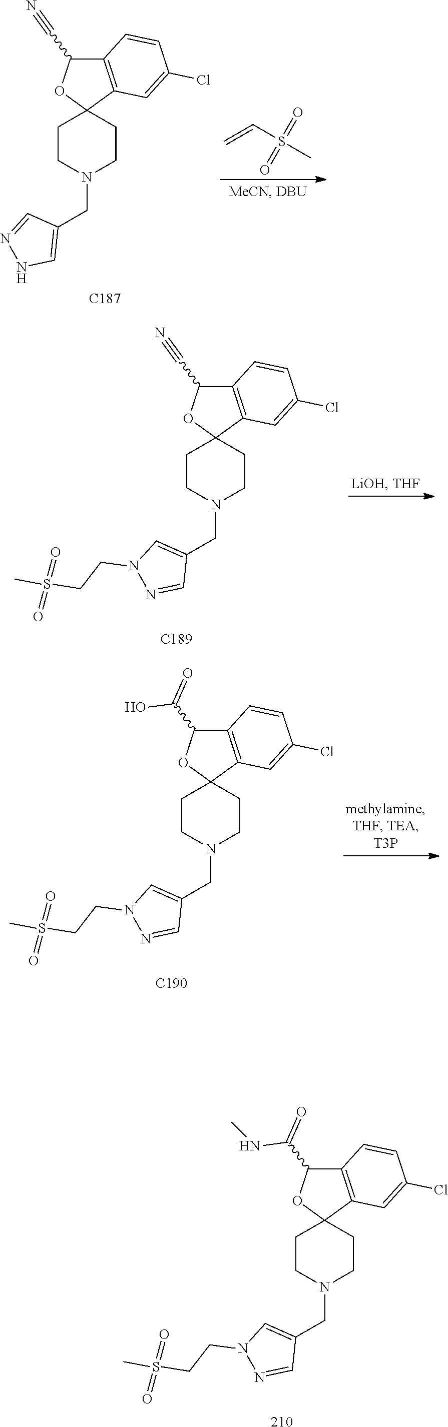

### Step 1. Preparation of 5-chloro-1′-[[1-(2-methylsulfonylethyl)pyrazol-4-yl]methyl]spiro[1H-isobenzofuran-3,4′-piperidine]-1-carbonitrile (C189)

To a stirred solution of 5-chloro-1′-(1H-pyrazol-4-ylmethyl)spiro[1H-isobenzofuran-3,4′-piperidine]-1-carbonitrile (400 mg, 0.8029 mmol) in MeCN (5.0 mL) was added DBU (302 mg, 0.3 mL, 1.96 mmol) followed by 1-methylsulfonylethylene (80.005 mg, 0.7462 mmol) at 0° C. The reaction was stirred at room temperature for 16 h, then the reaction mixture was diluted with water (10 mL), and extracted with ethyl acetate (2×50 mL). The organic layer was dried over sodium sulfate and concentrated to provide 5-chloro-1′-[[1-(2-methylsulfonylethyl)pyrazol-4-yl]methyl]spiro[1H-isobenzofuran-3,4′-piperidine]-1-carbonitrile (200 mg, 25%) ESI-MS m/z 434.87 [M+1]⁺.

### Step 2. Preparation of 5-chloro-1′-[[1-(2-methylsulfonylethyl)pyrazol-4-yl]methyl]spiro[1H-isobenzofuran-3,4′-piperidine]-1-carboxylic acid (C190)

To a stirred solution of 5-chloro-1′-[[1-(2-methylsulfonylethyl)pyrazol-4-yl]methyl]spiro[1H-isobenzofuran-3,4′-piperidine]-1-carbonitrile (100 mg, 0.1035 mmol) in water (1.0 mL) and THF (1.0 mL) was added LiOH (50 mg, 1.1796 mmol) at ambient temperature. The reaction mixture was stirred for 6 hours, and the reaction was concentrated, diluted with water (10 mL), acidified with 1 MHCl (pH=4) and extracted with ethyl acetate. Thye organic layer was dried over sodium sulfate and concentrated to provide 5-chloro-1′-[[1-(2-methylsulfonylethyl)pyrazol-4-yl]methyl]spiro[1H-isobenzofuran-3,4′-piperidine]-1-carboxylic acid (120 mg) ESI-MS m/z 454.32 [M+1]⁺.

### Step 3. Preparation of 5-chloro-N-methyl-1′-[[1-(2-methylsulfonylethyl)pyrazol-4-yl]methyl]spiro[1H-isobenzofuran-3,4′-piperidine]-1-carboxamide (210)

To a stirred solution of 5-chloro-1′-[[1-(2-methylsulfonylethyl)pyrazol-4-yl]methyl]spiro[1H-isobenzofuran-3,4′-piperidine]-1-carboxylic acid (120 mg, 0.2330 mmol) in THF (2.0 mL) were added methylamine (50 mg, 0.7331 mmol), TEA (143.75 mg, 0.2 mL, 1.4064 mmol) and T3P in Ethyl acetate (212.00 mg, 0.2 mL of 50% w/v, 0.3143 mmol) at ambient temperature and stirred for 16 hours. The reaction was diluted with water (10 mL), and extracted with ethyl acetate (2×50 mL). Organic layer was dried over sodium sulfate and concentrated. The crude compound was purified by reverse phase chromatography (Gradient: 10-100% MeCN in aqueous 10 mM ammonium bicarbonate) to provide 5-chloro-N-methyl-1′-[[1-(2-methylsulfonylethyl)pyrazol-4-yl]methyl]spiro[1H-isobenzofuran-3,4′-piperidine]-1-carboxamide (10 mg, 9%) ¹H NMR (400 MHz, DMSO-d6) δ: 7.70 (s, 1H), 7.58-7.57 (m, 1H), 7.41-7.40 (m, 2H), 7.37-7.32 (m, 2H), 5.38 (s, 1H), 4.51 (t, J=6.8 Hz, 2H), 3.68 (t, J=6.8 Hz, 2H), 3.41 (brs, 2H), 2.80 (s, 3H), 2.74 (brs, 2H), 2.61 (d, J=4.4 Hz, 3H), 2.50-2.49 (m, 1H), 2.29-2.24 (m, 1H), 2.08-2.00 (m, 1H), 1.85-1.78 (m, 2H), 1.62-1.58 (m, 1H). ESI-MS m z 467.21 [M+1]⁺.

### Compound 211

### 5-chloro-N-methyl-1′-[[1-(2-methylpyrimidin-5-yl)pyrazol-4-yl]methyl]spiro[1H-isobenzofuran-3,4′-piperidine]-1-carboxamide (211)

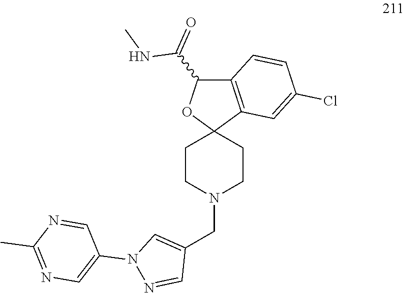 [SMILES: CNC(=O)C1OC2(CCN(Cc3cnn(-c4cnc(C)nc4)c3)CC2)c2cc(Cl)ccc21]

Compound 211 was prepared following the method described in step 2 and step 3 for the preparation of compound 210, starting from 6-chloro-1′-((1-(2-methylpyrimidin-5-yl)-1H-pyrazol-4-yl)methyl)-3H-spiro[isobenzofuran-1,4′-piperidine]-3-carbonitrile (C188). This provided 5-chloro-N-methyl-1′-[[1-(2-methylpyrimidin-5-yl)pyrazol-4-yl]methyl]spiro[1H-isobenzofuran-3,4′-piperidine]-1-carboxamide 211 (10 mg, 15%) ¹H NMR (400 MHz, DMSO-d6) δ: 9.17 (s, 2H), 8.55 (s, 1H), 7.79 (s, 1H), 7.61 (d, J=4.8 Hz, 1H), 7.40-7.32 (m, 3H), 5.40 (s, 1H), 3.56 (s, 2H), 2.80 (brs, 2H), 2.66 (s, 3H), 2.62 (d, J=4.8 Hz, 3H), 2.54-2.50 (m, 1H), 2.37-2.31 (m, 1H), 2.11-2.08 (m, 1H), 1.90-1.78 (m, 2H), 1.33-1.20 (m, 1H). ESI-MS m/z 453.2 [M+1]⁺.

Preparation S38

### 5-chlorospiro[1H-isobenzofuran-3,4′-piperidine]-1-carboxamide (S38)

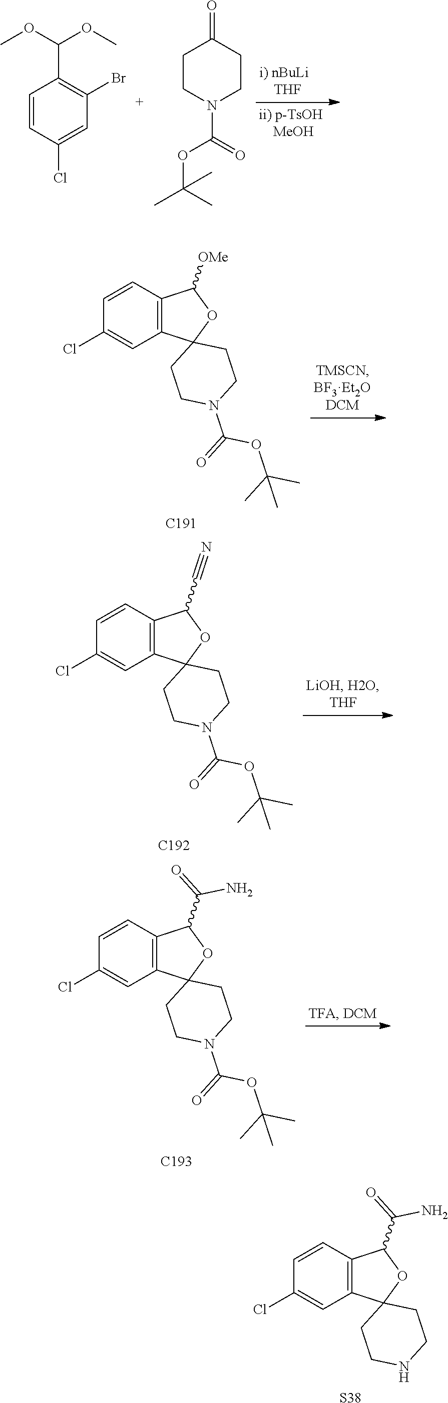

### Step 1. Synthesis of tert-butyl 5-chloro-1-methoxy-spiro[1H-isobenzofuran-3,4′-piperidine]-1′-carboxylate (C191)

To a solution of 2-bromo-4-chloro-1-(dimethoxymethyl)benzene (29.8 g, 112.2 mmol) in THF (230 mL) at −78° C. (dry ice-acetone bath) under N₂ was added nBuLi (48 mL of 2.8 M, 134.4 mmol). Stirred for 40 min at −78° C., then a solution of tert-butyl 4-oxopiperidine-1-carboxylate (24.7 g, 124.0 mmol) in THF (115 mL) was added. The reaction was stirred at −78° C. for 30 min, then warmed to 0° C. and stirred for 30 min at which point the reaction was quenched with saturated aqueous ammonium chloride (200 mL). Partitioned between MTBE and water (400 mL each). The organic layer was separated, washed with water then brine (500 mL each), dried over magnesium sulfate, filtered, and concentrated. The resulting residue was dissolved in MeOH (300 mL) treated with 4-methylbenzenesulfonic acid (Hydrate (1)) (350 mg, 1.840 mmol). Heated to 60° C. and stirred for 16 h. The reaction was quenched with saturated aqueous sodium bicarbonate (300 mL) then partitioned between water (200 mL) and MTBE (1 L). Organic layer was separated, washed with brine (300 mL) dried over magnesium sulfate, filtered, and concentrated. The resulting residue was purified by silica gel chromatography (Gradient: 0-50% EtOAc in heptane) to afford tert-butyl 5-chloro-1-methoxy-spiro[1H-isobenzofuran-3,4′-piperidine]-1′-carboxylate (25.8 g, 65% yield) as a viscous oil/glass. ¹H NMR (400 MHz, Chloroform-d) δ 7.30 (t, J=1.3 Hz, 2H), 7.10 (t, J=1.1 Hz, 1H), 6.02 (s, 1H), 4.12 (s, 1H), 3.48 (s, 3H), 3.28-3.14 (m, 2H), 1.97-1.72 (m, 3H), 1.65-1.55 (m, 2H), 1.49 (s, 9H).

### Step 2. Synthesis of tert-butyl 5-chloro-1-cyano-spiro[1H-isobenzofuran-3,4′-piperidine]-1′-carboxylate (C192)

To a solution of tert-butyl 5-chloro-1-methoxy-spiro[1H-isobenzofuran-3,4′-piperidine]-1′-carboxylate (3.7 g, 10.46 mmol) in DCM (50 mL) under N2 at −78° C. was added trimethylsilyl cyanide (2.0 mL, 15.00 mmol) followed by diethyloxonio(trifluoro)boranuide (1.4 mL, 11.34 mmol). Stirred at −78° C. for 30 min and then warmed to 0° C. After 1 h at 0° C. the reaction was quenched with saturated aqueous sodium bicarbonate (100 mL). The reaction was warmed to room temperature and treated with Boc₂O (1.1 g, 5.040 mmol). The resulting biphasic mixture was stirred for 1 h, then diluted with DCM (50 mL), then the layers were separated. The organic layer was washed successively with saturated aqueous sodium bicarbonate, 0.5 M aqueous NaOH, and brine (100 mL each), dried (MgSO₄), filtered, and concentrated. The resulting crude residue was purified by silica gel chromatography (Gradient: 0-50% EtOAc in heptane) to afford tert-butyl 5-chloro-1-cyano-spiro[1H-isobenzofuran-3,4′-piperidine]-1′-carboxylate (3.0 g, 82% yield) as a clear colorless glass. ¹H NMR (400 MHz, Chloroform-d) δ 7.39 (dd, J=8.2, 1.8 Hz, 1H), 7.35 (dt, J=8.2, 0.7 Hz, 1H), 7.14 (d, J=1.7 Hz, 1H), 5.85 (d, J=0.8 Hz, 1H), 4.25-4.02 (m, 2H), 3.31-3.04 (m, 2H), 2.00-1.76 (m, 3H), 1.74-1.62 (m, 1H), 1.49 (s, 9H).

### Step 3. Synthesis of tert-butyl 1-carbamoyl-5-chloro-spiro[1H-isobenzofuran-3,4′-piperidine]-1′-carboxylate (C193)

To a flask containing tert-butyl 5-chloro-1-cyano-spiro[1H-isobenzofuran-3,4′-piperidine]-1′-carboxylate (1.125 g, 2.580 mmol) was added THF (45 mL) and diluted with water (45 mL). To this solution was added lithium hydroxide mono-hydrate (237 mg, 5.648 mmol) and stirred at room temp overnight. Quenched the reaction into 100 mL of sat. aq NH₄Cl and DCM (100 mL). Extracted with DCM (3×), passed the pooled organics through a phase separator, and concentrated under reduced pressure. The crude residue was purified by silica gel chromatography (Gradient: 0-20% MeOH in DCM) to yield tert-butyl 1-carbamoyl-5-chloro-spiro[1H-isobenzofuran-3,4′-piperidine]-1′-carboxylate (962.7 mg, 92% yield) as a waxy foaming white solid. ¹H NMR (400 MHz, Chloroform-d) δ 7.59 (d, J=8.2 Hz, 1H), 7.33 (dd, J=8.2, 1.8 Hz, 1H), 7.09 (d, J=1.9 Hz, 1H), 6.61 (s, 1H), 5.49 (d, J=1.1 Hz, 1H), 5.46 (s, 1H), 4.20 (s, 2H), 3.22 (s, 2H), 2.03 (td, J=13.3, 5.1 Hz, 1H), 1.79 (d, J=16.0 Hz, 2H), 1.52 (s, 9H), 0.91 (t, J=6.7 Hz, 1H). LCMS m/z 311.1 [M+1]⁺.

### Step 4. Synthesis of 5-chlorospiro[1H-isobenzofuran-3,4′-piperidine]-1-carboxamide (S38)

To a flask containing tert-butyl 1-carbamoyl-5-chloro-spiro[1H-isobenzofuran-3,4′-piperidine]-1′-carboxylate (1.28 g, 3.140 mmol) was added DCM (10 mL) and TFA (2 mL, 25.96 mmol). The reaction was stirred for 3 hours. The reaction was concentrated and azeotroped with DCM and MTBE to yield crude 5-chlorospiro[1H-isobenzofuran-3,4′-piperidine]-1-carboxamide (Trifluoracetic acid (1))(1.19 g, 89% yield) which was used in further steps without additional purification.

¹H NMR (300 MHz, Chloroform-d) δ 9.36 (d, J=71.2 Hz, 2H), 7.64-7.59 (m, 1H), 7.40 (dd, J=8.2, 1.8 Hz, 1H), 7.21 (d, J=1.8 Hz, 1H), 6.68 (s, 1H), 6.33 (s, 1H), 5.55 (s, 1H), 3.70-3.39 (m, 4H), 2.53 (td, J=14.0, 5.3 Hz, 1H), 2.37-2.20 (m, 1H), 2.01 (t, J=15.4 Hz, 2H). LCMS m/z 267.18 [M+1]⁺.

### Compound 212

### 5-chloro-1′-[[1-[3-(trifluoromethyl)phenyl]pyrazol-4-yl]methyl]spiro[1H-isobenzofuran-3,4′-piperidine]-1-carboxamide (212)

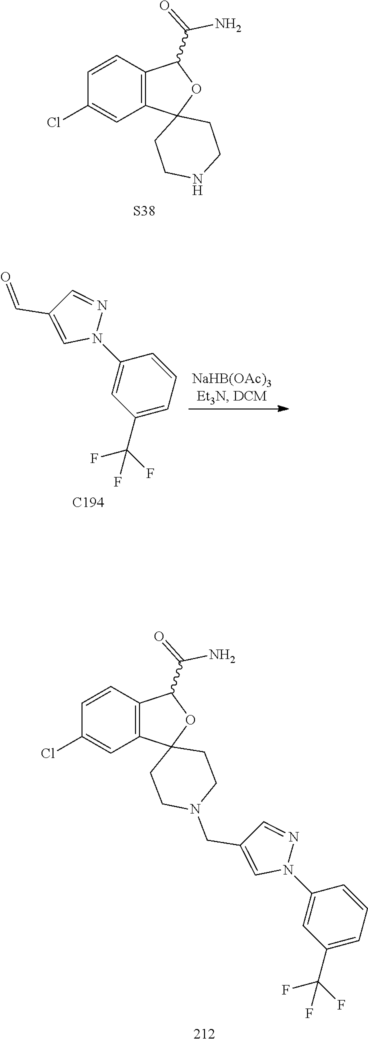 [SMILES: NC(=O)C1OC2(CCNCC2)c2cc(Cl)ccc21 ; "S38" ; O=Cc1cnn(-c2cccc(C(F)(F)F)c2)c1 ; "C194" ; NC(=O)C1OC2(CCN(Cc3cnn(-c4cccc(C(F)(F)F)c4)c3)CC2)c2cc(Cl)ccc21 ; "212"]

To a vial containing 5-chlorospiro[1H-isobenzofuran-3,4′-piperidine]-1-carboxamide (Hydrochloride salt) (8.8 mg, 0.02903 mmol) was added DCM (500 μL) and then 1-[3-(trifluoromethyl)phenyl]pyrazole-4-carbaldehyde (10 mg, 0.04164 mmol). To this was added Et₃N (20 μL, 0.1435 mmol) and then triacetoxyboranuide (Sodium salt) (22 mg, 0.1038 mmol) and stirred overnight. The reaction was quenched into sat. NaHCO₃ and extracted with DCM (3×). The pooled organics were passed through a phase separator and concentrated. The resulting crude residue was redissolved in DMSO and purified by reversed-phase HPLC. (Method: Waters XBridge Prep C8 Column; 30×150 mm, 5 micron. Gradient: Acetonitrile in Water with 10 mM Ammonium Hydroxide) to afford 5-chloro-1′-[[1-[3-(trifluoromethyl)phenyl]pyrazol-4-yl]methyl]spiro[1H-isobenzofuran-3,4′-piperidine]-1-carboxamide 212 (5.7 mg, 40% yield) ¹H NMR (400 MHz, DMSO-d6) δ 8.64 (s, 1H), 8.17 (d, J=9.4 Hz, 2H), 7.80-7.62 (m, 3H), 7.46-7.30 (m, 4H), 7.17 (s, 1H), 5.36 (s, 1H), 3.59 (s, 2H), 2.86 (s, 2H), 2.47-2.34 (m, 1H), 2.16-2.03 (m, 1H), 1.97-1.78 (m, 2H), 1.65 (d, J=13.4 Hz, 1H). LCMS m/z 491.32 [M+1]⁺.

### Compounds 213-276

Compounds 213-276 (see Table 17) were prepared from S38 and the appropriate aldehydes employing the same method as compound 212. All aryl bromides and piperidones were obtained from commercial sources or were synthesized as described above. Any modifications to methods are noted in Table 17 and accompanying footnotes.

TABLE 17
Structure and physicochemical data for Compounds 213-276

| Cmpd | Product | Aldehyde | LCMS m/z [M + H]⁺ |
|---|---|---|---|
| 213 | 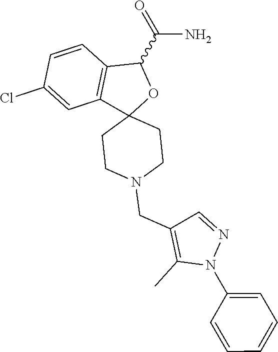 [SMILES: Cc1c(CN2CCC3(CC2)OC(C(N)=O)c2ccc(Cl)cc23)cnn1-c1ccccc1] | 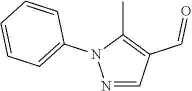 | ¹H NMR (400 MHz, DMSO-d6) δ 7.56 (s, 1H), 7.52 (d, J = 4.3 Hz, 4H), 7.48-7.31 (m, 5H), 7.15 (s, 1H), 5.36 (s, 1H), 3.44 (s, 2H), 2.87-2.78 (m, 2H), 2.54-2.31 (m, 2H), 2.31 (s, 3H), 2.06 (td, J = 12.8, 4.2 Hz, 1H), 1.94-1.75 (m, 2H), 1.63 (d, J = 13.2 Hz, 1H). LCMS m/z 437.32 [M + 1]⁺. |
| 214 | 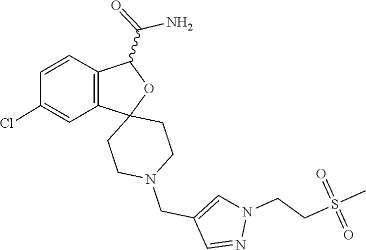 [SMILES: CS(=O)(=O)CCn1cc(CN2CCC3(CC2)OC(C(N)=O)c2ccc(Cl)cc23)cn1] | 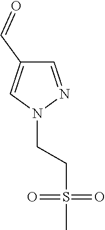 | ¹H NMR (400 MHz, DMSO-d6) δ: 7.07 (s, 1H), 7.41-7.33 (m, 4H), 7.10 (brs, 2H), 5.34 (s, 1H), 4.50 (t, J = 6.4 Hz, 2H), 3.68 (t, J = 6.8 Hz, 2H), 3.41 (brs, 2H), 2.81 (s, 3H), 2.73 (d, J = 8.4 Hz, 2H), 2.49-2.38 (m, 1H), 2.30-2.35 (m, 1H), 2.07-2.00 (m, 1H), 1.98- 1.00 (m, 3H). LCMS m/z 453.16 [M + 1]⁺. |
| 215 | 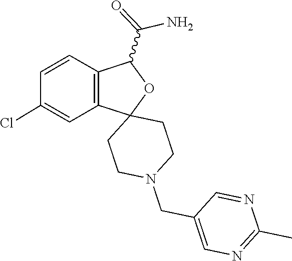 [SMILES: Cc1ncc(CN2CCC3(CC2)OC(C(N)=O)c2ccc(Cl)cc23)cn1] | 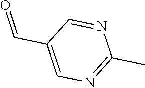 | ¹H NMR (400 MHz, DMSO-d6) δ 8.64 (s, 2H), 7.47-7.41 (m, 2H), 7.41-7.32 (m, 2H), 7.15 (s, 1H), 5.35 (s, 1H), 3.56 (s, 2H), 2.71 (d, J = 10.6 Hz, 2H), 2.61 (s, 3H), 2.37 (t, J = 11.7 Hz, 1H), 2.11-2.01 (m, 1H), 1.88 (td, J = 12.8, 4.2 Hz, 1H), 1.79 (d, J = 13.3 Hz, 1H), 1.61 (d, J = 13.3 Hz, 1H). LCMS m/z 373.25 [M + 1]⁺. |
| 216 | 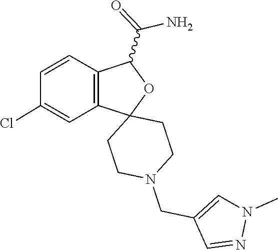 [SMILES: Cn1cc(CN2CCC3(CC2)OC(C(N)=O)c2ccc(Cl)cc23)cn1] | 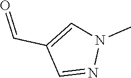 | ¹H NMR (400 MHz, DMSO-d6) δ 7.57 (s, 1H), 7.47-7.29 (m, 5H), 7.12 (s, 1H), 5.34 (s, 1H), 3.80 (s, 3H), 3.39 (s, 2H), 2.79-2.65 (m, 2H), 2.33 (dt, J = 52.9, 11.6 Hz, 2H), 2.02 (td, J = 12.9, 4.5 Hz, 1H), 1.90-1.75 (m, 2H), 1.60 (d, J = 13.3 Hz, 1H). LCMS m/z 361.32 [M + 1]⁺. |
| 217 | 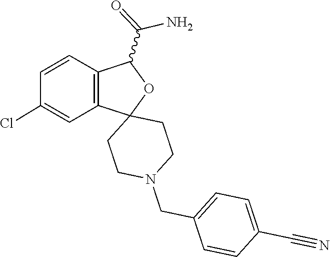 [SMILES: N#Cc1ccc(CN2CCC3(CC2)OC(C(N)=O)c2ccc(Cl)cc23)cc1] | 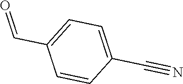 | ¹H NMR (400 MHz, DMSO-d6) δ 7.81 (d, J = 8.0 Hz, 2H), 7.59-7.31 (m, 6H), 7.15 (s, 1H), 5.35 (s, 1H), 3.64 (s, 2H), 2.74-2.66 (m, 2H), 2.60- 2.50 (m, 1H), 2.39 (t, J = 11.4 Hz, 1H), 2.16- 2.02 (m, 1H), 1.89 (td, J = 12.8, 4.2 Hz, 1H), 1.82- 1.58 (m, 2H). LCMS m/z 383.32 [M + 1]⁺. |
| 218 | 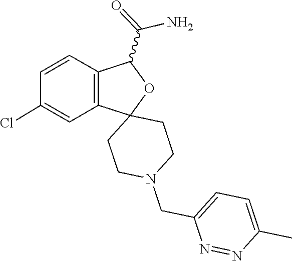 [SMILES: Cc1ccc(CN2CCC3(CC2)OC(C(N)=O)c2ccc(Cl)cc23)nn1] | 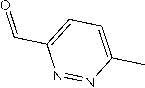 | ¹H NMR (400 MHz, DMSO-d6) δ 7.64 (d, J = 8.5 Hz, 1H), 7.55 (d, J = 8.6 Hz, 1H), 7.43 (d, J = 1.8 Hz, 2H), 7.41-7.32 (m, 2H), 7.17 (s, 1H), 5.36 (s, 1H), 3.82 (s, 2H), 2.72 (d, J = 10.7 Hz, 2H), 2.63 (d, J = 9.4 Hz, 1H), 2.60 (s, 3H), 2.45 (d, J = 11.7 Hz, 1H), 2.06 (td, J = 12.8, 4.7 Hz, 1H), 1.89 (td, J = 13.0, 4.4 Hz, 1H), 1.83-1.74 (m, 1H), 1.66- 1.56 (m, 1H). LCMS m/z 373.29 [M + 1]⁺. |
| 219 | 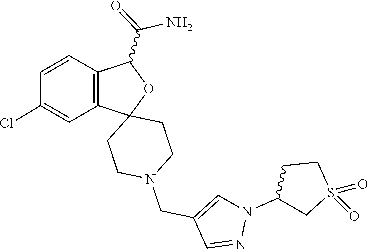 [SMILES: NC(=O)C1OC2(CCN(Cc3cnn(C4CCS(=O)(=O)C4)c3)CC2)c2cc(Cl)ccc21] | 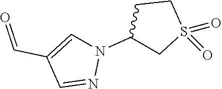 [SMILES: O=Cc1cnn(C2CCS(=O)(=O)C2)c1] | ¹H NMR (400 MHz, DMSO-d6) δ 7.78 (s, 1H), 7.48-7.31 (m, 5H), 7.12 (s, 1H), 5.35 (s, 1H), 5.21 (p, J = 7.6 Hz, 1H), 3.71 (dd, J = 13.7, 8.2 Hz, 1H), 3.48-3.41 (m, 1H), 3.40 (s, 2H), 3.24 (dt, J = 13.1, 8.1 Hz, 1H), 2.78-2.69 (m, 2H), 2.61 (dq, J = 13.1, 6.7 Hz, 1H), 2.55-2.46 (m, 1H), 2.46-2.35 (m, 1H), 2.28 (t, J = 11.8 Hz, 1H), 2.07- 1.97 (m, 1H), 1.90- |
| | | | 1.74 (m, 2H), 1.60 (d, J = |
| | | | 13.2 Hz, 1H). 1H under |
| | | | H₂O LCMS m/z 465.31 |
| | | | [M + 1]⁺. |
| 220 | 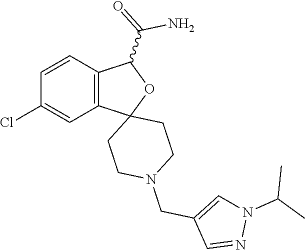 [SMILES: CC(C)n1cc(CN2CCC3(CC2)OC(C(N)=O)c2ccc(Cl)cc23)cn1] | 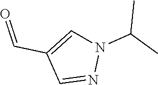 | ¹H NMR (400 MHz, DMSO-d6): δ 7.63 (s, 1H), 7.41-7.32 (m, 5H), 7.10 (brs, 1H), 5.36 (s, 1H), 4.47-4.40 (m, 1H), 3.38 (s, 2H), 2.75-2.67 (m, 1H), 2.49-2.24 (m, 2H), 2.07-2.00 (m, 1H), 1.98-1.80 (m, 2H), 1.62- 1.55 (m, 2H), 1.39 (d, J = 6.8 Hz, 6H). LCMS m/z 389.33 [M + 1]⁺. |
| 221 | 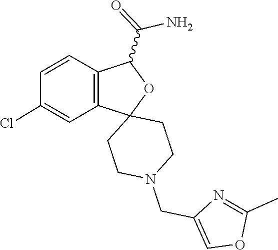 [SMILES: Cc1nc(CN2CCC3(CC2)OC(C(N)=O)c2ccc(Cl)cc23)co1] | 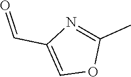 | ¹H NMR (400 MHz, DMSO-d6) δ 7.81 (s, 1H), 7.48-7.31 (m, 4H), 7.14 (s, 1H), 5.35 (s, 1H), 3.41 (s, 2H), 2.84-2.74 (m, 2H), 2.47-2.31 (m, 4H), 2.05 (dt, J = 13.0, 6.0 Hz, 1H), 1.94-1.72 (m, 2H), 1.61 (d, J = 13.2 Hz, 1H). LCMS m/z 362.23 [M + 1]⁺. |
| 222 | 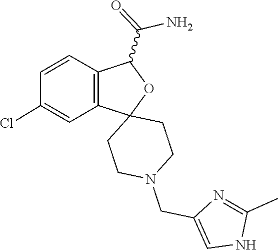 [SMILES: Cc1nc(CN2CCC3(CC2)OC(C(N)=O)c2ccc(Cl)cc23)c[nH]1] | 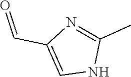 | ¹H NMR (400 MHz, DMSO-d6) δ 7.47-7.30 (m, 4H), 7.15 (s, 1H), 5.35 (s, 1H), 3.44 (s, 2H), 2.85-2.69 (m, 2H), 2.54- 2.43 (m, 1H), 2.35 (t, J = 11.7 Hz, 1H), 2.13 (s, 3H), 2.01 (td, J = 12.8, 3.5 Hz, 1H), 1.93-1.76 (m, 3H), 1.61 (d, J = 13.4 Hz, 1H). 1H exchangeable not seen. LCMS m/z 361.2 [M + 1]⁺. |
| 223 | 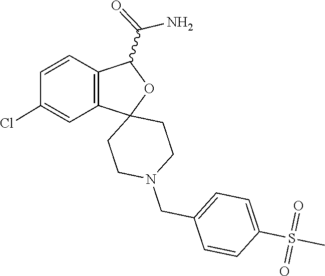 [SMILES: CS(=O)(=O)c1ccc(CN2CCC3(CC2)OC(C(N)=O)c2ccc(Cl)cc23)cc1] | 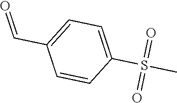 | ¹H NMR (400 MHz, DMSO-d6) δ 7.90 (d, J = 8.2 Hz, 2H), 7.63 (d, J = 8.1 Hz, 2H), 7.48-7.29 (m, 4H), 7.18-7.13 (m, 1H), 5.36 (s, 1H), 3.66 (s, 2H), 3.21 (s, 3H), 2.72 (d, J = 10.4 Hz, 2H), 2.61- 2.30 (m, 2H), 2.09 (td, J = 12.9, 3.8 Hz, 1H), 1.90 (td, J = 12.8, 4.1 Hz, 1H), 1.84-1.57 (m, 2H). LCMS m/z 435.2 [M + 1]⁺. |
| 224 | 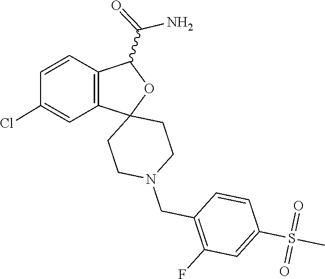 [SMILES: CS(=O)(=O)c1ccc(CN2CCC3(CC2)OC(C(N)=O)c2ccc(Cl)cc23)c(F)c1] | 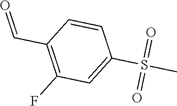 | LCMS m/z 453.21 [M + 1]⁺. |
| 225 | 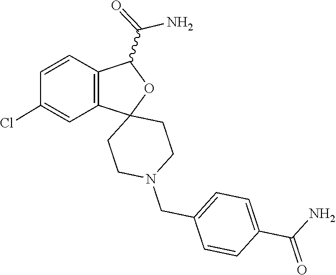 [SMILES: NC(=O)c1ccc(CN2CCC3(CC2)OC(C(N)=O)c2ccc(Cl)cc23)cc1] | 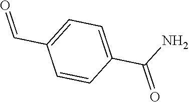 [SMILES: NC(=O)c1ccc(C=O)cc1] | ¹H NMR (400 MHz, DMSO-d6) δ 7.94 (s, 1H), 7.84 (d, J = 7.9 Hz, 2H), 7.46-7.40 (m, 4H), 7.39-7.30 (m, 3H), 7.14 (s, 1H), 5.35 (s, 1H), 3.60 (s, 2H), 2.72 (d, J = 10.8 Hz, 2H), 2.37 (t, J = 11.7 Hz, 1H), 2.07 (td, J = 13.1, 4.5 Hz, 1H), 1.89 (td, J = 13.1, 4.4 Hz, 1H), 1.70 (dd, J = 71.4, 13.2 Hz, 2H). LCMS m/z 400.21 [M + 1]⁺. |
| 226 | 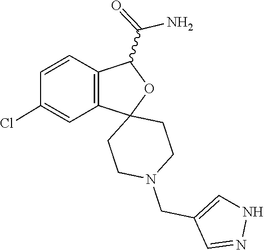 [SMILES: NC(=O)C1OC2(CCN(Cc3cn[nH]c3)CC2)c2cc(Cl)ccc21] | 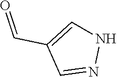 | ¹H NMR (300 MHz, Chloroform-d) δ 7.64- 7.50 (m, 3H), 7.30 (d, J = 1.8 Hz, 1H), 7.09 (d, J = 1.8 Hz, 1H), 6.63 (s, 1H), 5.43 (s, 1H), 5.40 (s, 1H), 3.59 (s, 2H), 3.21 (s, 1H), 2.91 (d, J = 14.1 Hz, 2H), 2.45 (s, 2H), 2.12 (d, J = 16.7 Hz, 1H), 2.01-1.86 (m, 1H), 1.79 (t, J = 12.0 Hz, 2H). LCMS m/z 347.29 [M + 1]⁺. |
| 227 | 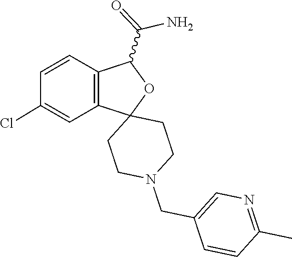 [SMILES: Cc1ccc(CN2CCC3(CC2)OC(C(N)=O)c2ccc(Cl)cc23)cn1] | 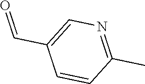 | ¹H NMR (400 MHz, DMSO-d6) δ 8.38 (s, 1H), 7.63 (d, J = 7.9 Hz, 1H), 7.45-7.31 (m, 4H), 7.23 (d, J = 8.0 Hz, 1H), 7.16 (s, 1H), 5.35 (s, 1H), 3.56 (s, 2H), 2.72 (d, J = 7.0 Hz, 2H), 2.45 (s, 3H), 2.41-1.97 (m, 2H), 1.94- 1.73 (m, 2H), 1.61 (d, J = 13.3 Hz, 1H). LCMS m/z 372.25 [M + 1]⁺. |
| 228 | 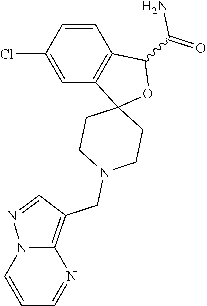 [SMILES: NC(=O)C1OC2(CCN(Cc3cnn4cccnc34)CC2)c2cc(Cl)ccc21] | 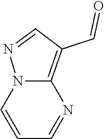 [SMILES: O=Cc1cnn2cccnc12] | ¹H NMR (400 MHz, DMSO-d6) δ 9.08 (dd, J = 7.1, 1.7 Hz, 1H), 8.55 (dd, J = 4.1, 1.8 Hz, 1H), 8.20 (s, 1H), 7.42-7.30 (m, 4H), 7.12 (s, 1H), 7.02 (dd, J = 7.1, 4.0 Hz, 1H), 5.32 (s, 1H), 3.80 (s, 2H), 2.84-2.76 (m, 2H), 2.50-2.29 (m, 2H), 2.04 (td, J = 12.7, 4.4 Hz, 1H), 1.90-1.75 (m, 2H), 1.64- 1.56 (m, 1H). LCMS m/z 397.858 [M + 1]⁺. |
| 229 | 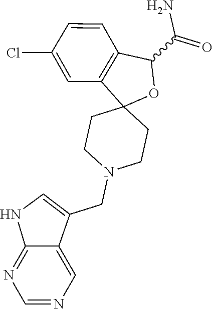 [SMILES: NC(=O)C1OC2(CCN(Cc3c[nH]c4ncncc34)CC2)c2cc(Cl)ccc21] | 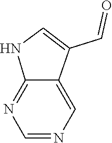 [SMILES: O=Cc1c[nH]c2ncncc12] | ¹H NMR (400 MHz, DMSO-d6) δ 11.96 (s, 1H), 9.15 (s, 1H), 8.74 (s, 1H), 7.47-7.29 (m, 5H), 7.12 (s, 1H), 5.34 (s, 1H), 3.75 (s, 2H), 2.81 (d, J = 11.0 Hz, 2H), 2.50-2.29 (m, 2H), 2.13-2.01 (m, 1H), 1.89 (td, J = 13.0, 4.4 Hz, 1H), 1.83-1.56 (m, 2H). LCMS m/z 398.22 [M + 1]⁺. |
| 230 | 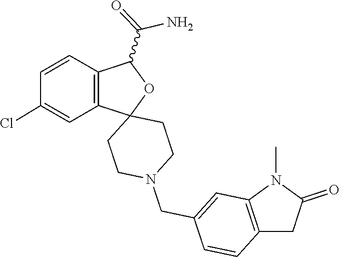 [SMILES: CN1C(=O)Cc2ccc(CN3CCC4(CC3)OC(C(N)=O)c3ccc(Cl)cc34)cc21] | 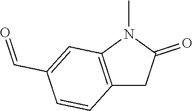 | ¹H NMR (400 MHz, DMSO-d6) δ 7.53-7.29 (m, 5H), 7.25-6.91 (m, 3H), 5.34 (d, J = 6.8 Hz, 1H), 3.53 (d, J = 14.2 Hz, 3H), 3.12 (s, 2H), 2.75 (d, J = 10.2 Hz, 2H), 2.49- 2.22 (m, 2H), 2.12-2.02 (m, 1H), 1.95-1.73 (m, 2H), 1.68-1.56 (m, 1H). 2H under solvent LCMS m/z 426.26 [M + 1]⁺. |
| 231 | 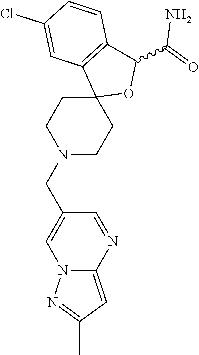 [SMILES: Cc1cc2ncc(CN3CCC4(CC3)OC(C(N)=O)c3ccc(Cl)cc34)cn2n1] | 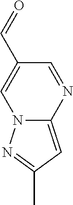 | ¹H NMR (400 MHz, DMSO-d6) δ 8.90 (d, J = 2.1 Hz, 1H), 8.48 (d, J = 2.1 Hz, 1H), 7.54-7.28 (m, 4H), 7.15 (s, 1H), 6.49 (s, 1H), 5.35 (s, 1H), 3.63 (s, 2H), 2.79 (d, J = 10.7 Hz, 2H), 2.64-2.31 (m, 2H), 2.41 (s, 3H), 2.08 (td, J = 13.1, 4.6 Hz, 1H), 1.89 (td, J = 14.2, 13.6, 5.1 Hz, 1H), 1.84- 1.59 (m, 2H). LCMS m/z 412.3 [M + 1]⁺. |
| 232 | 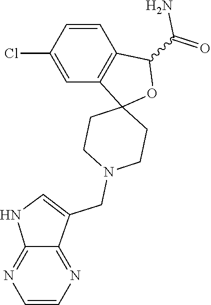 [SMILES: NC(=O)C1OC2(CCN(Cc3c[nH]c4nccnc34)CC2)c2cc(Cl)ccc21] | 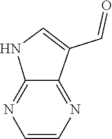 [SMILES: O=Cc1c[nH]c2nccnc12] | ¹H NMR (400 MHz, DMSO-d6) δ 11.96 (s, 1H), 8.39 (d, J = 2.6 Hz, 1H), 8.23 (d, J = 2.7 Hz, 1H), 7.79 (d, J = 2.6 Hz, 1H), 7.42-7.26 (m, 4H), 7.11 (s, 1H), 5.32 (s, 1H), 3.79 (s, 2H), 2.83 (d, J = 11.0 Hz, 2H), 2.52-2.31 (m, 2H), 2.04 (td, J = 12.8, 4.4 Hz, 1H), 1.83 (dt, J = 20.8, 13.8 Hz, 2H), 1.60 (d, J = 13.1 Hz, 1H). LCMS m/z 398.26 [M + 1]⁺. |
| 233 | 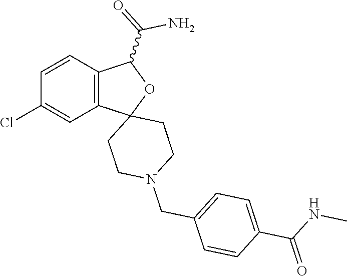 [SMILES: CNC(=O)c1ccc(CN2CCC3(CC2)OC(C(N)=O)c2ccc(Cl)cc23)cc1] | 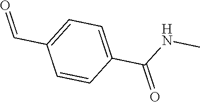 | ¹H NMR (400 MHz, DMSO-d6) δ 8.40 (d, J = 4.8 Hz, 1H), 7.80 (d, J = 8.0 Hz, 2H), 7.51-7.32 (m, 6H), 7.15 (s, 1H), 5.36 (s, 1H), 3.59 (s, 2H), 2.78 (d, J = 4.5 Hz, 3H), 2.73 (d, J = 11.1 Hz, 3H), 2.37 (t, J = 11.8 Hz, 1H), 2.08 (td, J = 13.0, 4.5 Hz, 1H), 1.89 (td, J = 13.0, 4.4 Hz, 1H), 1.83-1.54 (m, 2H). LCMS m/z 414.25 [M + 1]⁺. |
| 234 | 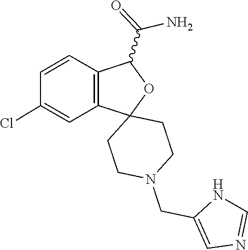 [SMILES: NC(=O)C1OC2(CCN(Cc3cnc[nH]3)CC2)c2cc(Cl)ccc21] | 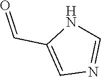 | ¹H NMR (400 MHz, DMSO-d6) δ 7.55 (s, 1H), 7.46-7.29 (m, 4H), 7.13 (s, 1H), 6.88 (s, 1H), 5.35 (s, 1H), 3.48 (s, 2H), 2.76 (d, J = 10.5 Hz, 2H), 2.47-2.29 (m, 2H), 2.02 (td, J = 12.9, 4.5 Hz, 1H), 1.84 (ddd, J = 23.9, 15.1, 11.4 Hz, 2H), 1.60 (d, J = 13.3 Hz, 1H). LCMS m/z 347.28 [M + 1]⁺. |
| 235 | 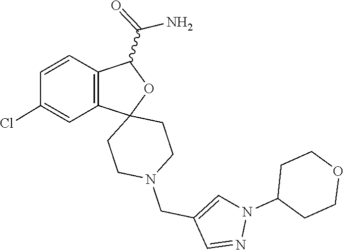 [SMILES: NC(=O)C1OC2(CCN(Cc3cnn(C4CCOCC4)c3)CC2)c2cc(Cl)ccc21] | 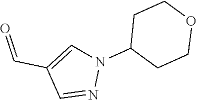 [SMILES: O=Cc1cnn(C2CCOCC2)c1] | ¹H NMR (400 MHz, DMSO-d6) δ 7.67 (s, 1H), 7.42-7.33 (m, 5H), 7.10 (brs, 1H), 5.34 (s, 1H), 4.35-4.31 (m, 1H), 3.95-3.92 (m, 2H), 3.473.41 (m, 4H), 2.75- 2.73 (m, 2H), 2.50-2.49 (m, 1H), 2.43-2.02 (m, 1H), 2.00-1.77 (m, 7H), 1.60 (d, J = 11.2 Hz, 1H). LCMS m/z 431.2 [M + 1]⁺. |
| 236 | 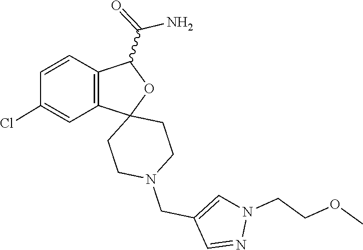 [SMILES: COCCn1cc(CN2CCC3(CC2)OC(C(N)=O)c2ccc(Cl)cc23)cn1] | 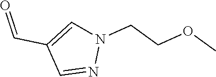 | ¹H NMR (400 MHz, DMSO-d6) δ 7.59 (s, 1H), 7.47-7.30 (m, 5H), 7.12 (s, 1H), 5.34 (s, 1H), 4.21 (t, J = 5.4 Hz, 2H), 3.66 (t, J = 5.3 Hz, 2H), 3.40 (s, 2H), 3.21 (s, 3H), 2.81-2.70 (m, 2H), 2.48- 2.23 (m, 2H), 2.03 (td, J = 12.9, 4.5 Hz, 1H), 1.93- 1.76 (m, 2H), 1.67- 1.54 (m, 1H). LCMS m/z 405.3 [M + 1]⁺. |
| 237 | 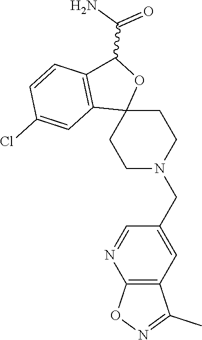 [SMILES: Cc1noc2ncc(CN3CCC4(CC3)OC(C(N)=O)c3ccc(Cl)cc34)cc12] | 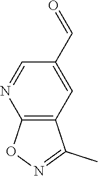 | ¹H NMR (400 MHz, DMSO-d6) δ 8.62 (d, J = 2.1 Hz, 1H), 8.33 (d, J = 2.1 Hz, 1H), 7.52-7.30 (m, 4H), 7.16 (s, 1H), 5.36 (s, 1H), 3.73 (s, 2H), 2.76 (d, J = 11.0 Hz, 2H), 2.59 (s, 3H), 2.42 (t, J = 11.4 Hz, 1H), 2.06 (dt, J = 13.0, 7.8 Hz, 1H), 1.89 (td, J = 12.9, 4.3 Hz, 1H), 1.84-1.59 (m, 2H). LCMS m/z 413.25 [M + 1]⁺. |
| 238 | 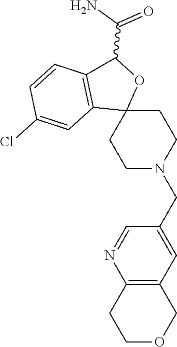 [SMILES: NC(=O)C1OC2(CCN(Cc3cnc4c(c3)COCC4)CC2)c2cc(Cl)ccc21] | 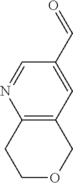 [SMILES: O=Cc1cnc2c(c1)COCC2] | ¹H NMR (400 MHz, DMSO-d6) δ 8.33 (d, J = 2.1 Hz, 1H), 7.46-7.31 (m, 5H), 7.15 (s, 1H), 5.35 (s, 1H), 4.71 (s, 2H), 3.97 (t, J = 5.8 Hz, 2H), 3.52 (s, 2H), 2.86 (t, J = 5.9 Hz, 2H), 2.78-2.65 (m, 2H), 2.58-2.29 (m, 2H), 2.05 (td, J = 12.8, 4.5 Hz, 1H), 1.87 (td, J = 12.9, 4.3 Hz, 1H), 1.82- 1.56 (m, 2H). LCMS m/z 414.25 [M + 1]⁺. |
| 239 | 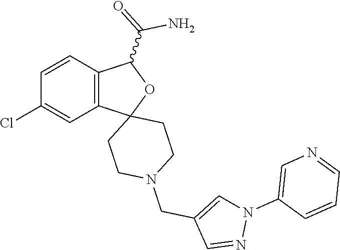 [SMILES: NC(=O)C1OC2(CCN(Cc3cnn(-c4cccnc4)c3)CC2)c2cc(Cl)ccc21] | 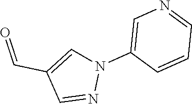 [SMILES: O=Cc1cnn(-c2cccnc2)c1] | ¹H NMR (400 MHz, DMSO-d6) δ 9.10 (d, J = 2.4 Hz, 1H), 8.53-8.52 (m, 1H), 8.50 (dd, J1 = 1.2 Hz, J2 = 4.8 Hz, 1H), 8.24-8.21 (m, 1H), 7.75 (s, 1H), 7.55-7.51 (m, 1H), 7.42-7.40 (m, 2H), 7.39-7.37 (m, 1H), 7.36 (d, J = 1.2 Hz, 1H), 7.33 (d, J = 2.0 Hz, 1H), 7.12 (brs, 1H), 5.35 (s, 1H), 3.61 (s, 2H), 2.81-2.79 (m, 2H), 2.49-2.46 (m, 1H), 2.35 (t, J = 9.6 Hz, 1H), 2.10-2.02 (m, 1H), |
| | | | 1.92-1.86 (m, 1H), 1.70- |
| | | | 1.62 (m, 1H). LCMS m/z |
| | | | 424.16 [M + 1]⁺. |
| 240 | 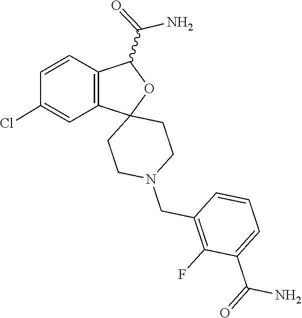 [SMILES: NC(=O)c1cccc(CN2CCC3(CC2)OC(C(N)=O)c2ccc(Cl)cc23)c1F] | 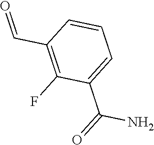 [SMILES: NC(=O)c1cccc(C=O)c1F] | ¹H NMR (400 MHz, DMSO-d6) δ 7.67 (d, J = 52.6 Hz, 2H), 7.59-7.50 (m, 2H), 7.47-7.40 (m, 2H), 7.41-7.31 (m, 2H), 7.24 (t, J = 7.6 Hz, 1H), 7.18-7.08 (m, 1H), 5.36 (s, 1H), 3.62 (s, 2H), 2.76 (d, J = 11.0 Hz, 2H), 2.41 (s, 2H), 2.07 (td, J = 12.8, 4.3 Hz, 1H), 1.89 (td, J = 12.9, 4.2 Hz, 1H), 1.71 (dd, J = 73.6, 13.2 Hz, 2H). LCMS m/z 418.22 [M + 1]⁺. |
| 241 | 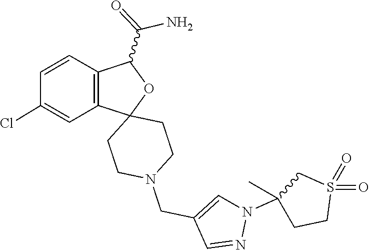 [SMILES: CC1(n2cc(CN3CCC4(CC3)OC(C(N)=O)c3ccc(Cl)cc34)cn2)CCS(=O)(=O)C1] | 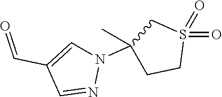 | ¹H NMR (400 MHz, DMSO-d6) δ 7.92 (s, 1H), 7.49-7.30 (m, 5H), 7.12 (s, 1H), 5.35 (s, 1H), 4.06 (d, J = 14.0 Hz, 1H), 3.51 (d, J = 14.0 Hz, 1H), 3.41 (s, 2H), 3.41-3.34 (m, 1H), 3.12 (q, J = 12.5, 10.5 Hz, 1H), 3.02- 2.92 (m, 1H), 2.83-2.68 (m, 2H), 2.49-2.22 (m, 3H), 2.03 (td, J = 13.1, 4.4 Hz, 1H), 1.94-1.74 (m, 2H), 1.66 (s, 3H), 1.61 (d, J = 14.0 Hz, 1H), |
| | | | LCMS m/z 479.27 |
| | | | [M + 1]⁺. |
| 242 | 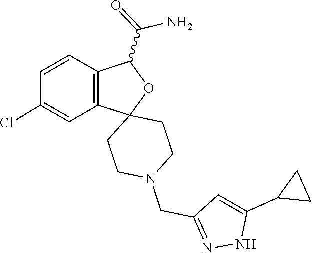 [SMILES: NC(=O)C1OC2(CCN(Cc3cc(C4CC4)[nH]n3)CC2)c2cc(Cl)ccc21] | 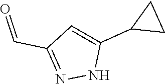 | ¹H NMR (400 MHz, DMSO-d6) δ 12.24 (s, 1H), 7.51-7.27 (m, 4H), 7.13 (s, 1H), 5.82 (s, 1H), 5.35 (s, 1H), 3.45 (s, 2H), 2.80-2.64 (m, 2H), 2.47- 2.25 (m, 2H), 2.10- 1.96 (m, 1H), 1.92-1.72 (m, 3H), 1.60 (d, J = 13.3 Hz, 1H), 0.85 (d, J = 8.2 Hz, 2H), 0.62 (dd, J = 5.2, 2.0 Hz, 2H). LCMS m/z 387.29 [M + 1]⁺. |
| 243 | 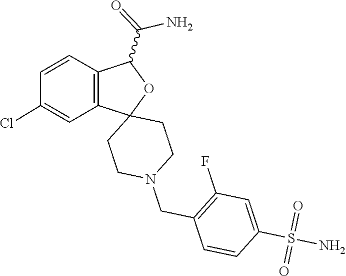 [SMILES: NC(=O)C1OC2(CCN(Cc3ccc(S(N)(=O)=O)cc3F)CC2)c2cc(Cl)ccc21] | 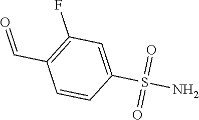 [SMILES: NS(=O)(=O)c1ccc(C=O)c(F)c1] | ¹H NMR (400 MHz, DMSO-d6) δ 7.67 (d, J = 5.8 Hz, 2H), 7.59 (d, J = 9.5 Hz, 1H), 7.51-7.41 (m, 4H), 7.40-7.30 (m, 2H), 7.17 (s, 1H), 5.35 (s, 1H), 3.67 (s, 2H), 2.80- 2.68 (m, 2H), 2.63-2.33 (m, 2H), 2.15-2.01 (m, 1H), 1.89 (td, J = 12.8, 4.1 Hz, 1H), 1.81 (d, J = 13.0 Hz, 1H), 1.62 (d, J = 13.3 Hz, 1H). LCMS m/z 454.21 [M + 1]⁺. |
| 244 | 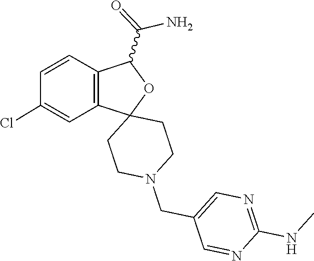 [SMILES: CNc1ncc(CN2CCC3(CC2)OC(C(N)=O)c2ccc(Cl)cc23)cn1] | 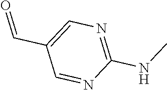 | ¹H NMR (400 MHz, DMSO-d6) δ 8.20 (s, 2H), 7.43 (d, J = 1.9 Hz, 2H), 7.40-7.31 (m, 2H), 7.14 (s, 1H), 7.04-6.99 (m, 1H), 5.35 (s, 1H), 3.36 (s, 2H), 2.78 (d, J = 4.7 Hz, 3H), 2.71 (d, J = 10.5 Hz, 2H), 2.48-2.26 (m, 2H), 2.04 (td, J = 12.9, 4.5 Hz, 1H), 1.90- 1.75 (m, 2H), 1.60 (d, J = 13.4 Hz, 1H). LCMS m/z 388.24 [M + 1]⁺. |
| 245 | 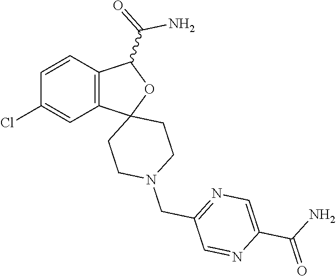 [SMILES: NC(=O)c1cnc(CN2CCC3(CC2)OC(C(N)=O)c2ccc(Cl)cc23)cn1] | 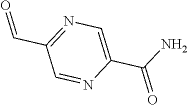 [SMILES: NC(=O)c1cnc(C=O)cn1] | ¹H NMR (300 MHz, DMSO-d6) δ 9.12 (d, J = 1.4 Hz, 1H), 8.77 (d, J = 1.4 Hz, 1H), 8.22 (s, 1H), 7.82 (s, 1H), 7.50-7.32 (m, 4H), 7.16 (s, 1H), 5.36 (s, 1H), 3.83 (s, 2H), 2.85-2.58 (m, 4H), 2.31- 1.55 (m, 4H). LCMS m/z 402.24 [M + 1]⁺. |
| 246 | 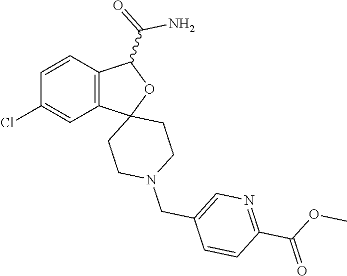 [SMILES: COC(=O)c1ccc(CN2CCC3(CC2)OC(C(N)=O)c2ccc(Cl)cc23)cn1] | 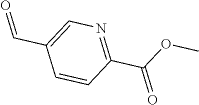 | ¹H NMR (400 MHz, DMSO-d6) δ 8.68 (d, J = 2.1 Hz, 1H), 8.05 (d, J = 8.0 Hz, 1H), 7.95 (dd, J = 8.0, 2.1 Hz, 1H), 7.49- 7.31 (m, 4H), 7.16 (s, 1H), 5.36 (s, 1H), 3.88 (s, 3H), 3.66 (d, J = 15.7 Hz, 2H), 2.72 (d, J = 10.9 Hz, 2H), 2.61-2.29 (m, 2H), 2.08 (td, J = 12.4, 3.4 Hz, 1H), 1.90 (td, J = 13.1, 12.3, 4.1 Hz, 1H), 1.80 (d, J = 13.2 Hz, 1H), 1.62 (d, J = 13.3 Hz, 1H). LCMS m/z 416.19 [M + 1]⁺. |
| 247 | 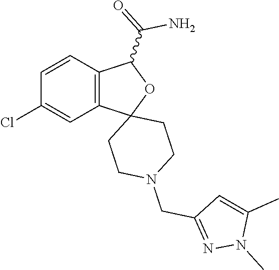 [SMILES: Cc1cc(CN2CCC3(CC2)OC(C(N)=O)c2ccc(Cl)cc23)nn1C] | 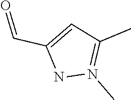 | ¹H NMR (400 MHz, DMSO-d6) δ 7.49-7.29 (m, 4H), 7.19-7.03 (m, 1H), 5.93 (s, 1H), 5.34 (s, 1H), 3.65 (s, 3H), 3.39 (s, 2H), 2.74 (d, J = 11.2 Hz, 2H), 2.50-2.25 (m, 2H), 2.21 (s, 3H), 2.01 (td, J = 12.6, 4.0 Hz, 1H), 1.90- 1.73 (m, 2H), 1.59 (d, J = 13.8 Hz, 1H). LCMS m/z 375.24 [M + 1]⁺. |
| 248 | 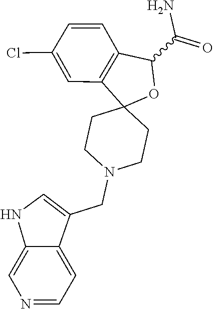 [SMILES: NC(=O)C1OC2(CCN(Cc3c[nH]c4cnccc34)CC2)c2cc(Cl)ccc21] | 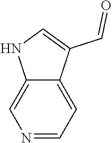 [SMILES: O=Cc1c[nH]c2cnccc12] | ¹H NMR (300 MHz, DMSO-d6) δ 11.45 (s, 1H), 8.70 (s, 1H), 8.09 (d, J = 5.4 Hz, 1H), 7.65 (d, J = 5.5 Hz, 1H), 7.50 (d, J = 2.4 Hz, 1H), 7.45- 7.31 (m, 4H), 7.10 (s, 1H), 5.33 (s, 1H), 3.71 (s, 2H), 2.86-2.69 (m, 2H), 2.45-2.29 (m, 2H), 2.07- 1.53 (m, 4H). LCMS m/z 397.23 [M + 1]⁺. |
| 249 | 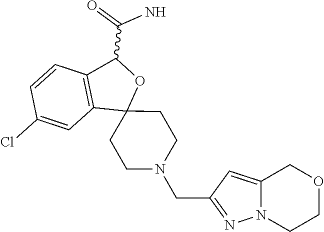 [SMILES: NC(=O)C1OC2(CCN(Cc3cc4n(n3)CCOC4)CC2)c2cc(Cl)ccc21] | 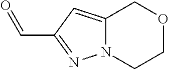 [SMILES: O=Cc1cc2n(n1)CCOC2] | ¹H NMR (400 MHz, DMSO-d6) δ 7.47-7.31 (m, 4H), 7.13 (s, 1H), 5.97 (s, 1H), 5.35 (s, 1H), 4.77 (s, 2H), 4.04 (s, 4H), 3.47 (s, 2H), 2.76 (d, J = 11.0 Hz, 2H), 2.48-2.27 (m, 2H), 2.03 (td, J = 12.9, 4.5 Hz, 1H), 1.92- 1.55 (m, 3H). LCMS m/z 403.23 [M + 1]⁺. |
| 250 | 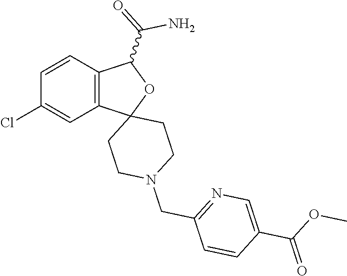 [SMILES: COC(=O)c1ccc(CN2CCC3(CC2)OC(C(N)=O)c2ccc(Cl)cc23)nc1] | 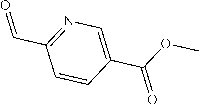 | ¹H NMR (300 MHz, DMSO-d6) δ 9.02 (d, J = 1.7 Hz, 1H), 8.30 (dd, J = 8.1, 2.4 Hz, 1H), 7.68 (d, J = 8.2 Hz, 1H), 7.51- 7.30 (m, 4H), 7.15 (s, 1H), 5.36 (s, 1H), 3.89 (s, 3H), 3.76 (s, 2H), 2.83- 2.55 (m, 3H), 2.18-1.55 (m, 5H). LCMS m/z 416.19 [M + 1]⁺. |
| 251 | 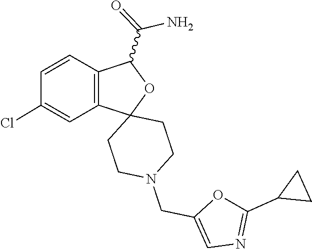 [SMILES: NC(=O)C1OC2(CCN(Cc3cnc(C4CC4)o3)CC2)c2cc(Cl)ccc21] | 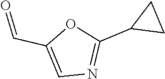 | ¹H NMR (400 MHz, DMSO-d6) δ 7.43 (t, J = 2.5 Hz, 2H), 7.41-7.31 (m, 2H), 7.15 (s, 1H), 6.86 (s, 1H), 5.35 (s, 1H), 3.55 (s, 2H), 2.77-2.69 (m, 2H), 2.55-2.28 (m, 2H), 2.13-2.01 (m, 2H), 1.88 (td, J = 14.0, 13.5, 4.8 Hz, 1H), 1.82-1.57 (m, 2H), 1.05-0.85 (m, 4H). LCMS m/z 388.24 [M + 1]⁺. |
| 252 | 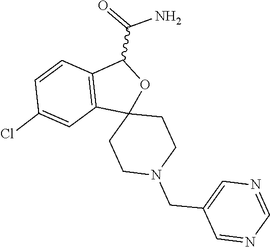 [SMILES: NC(=O)C1OC2(CCN(Cc3cncnc3)CC2)c2cc(Cl)ccc21] | 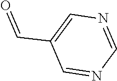 | ¹H NMR (400 MHz, DMSO-d6) δ 9.11 (s, 1H), 8.77 (s, 2H), 7.48- 7.31 (m, 4H), 7.16 (s, 1H), 5.36 (s, 1H), 3.62 (s, 2H), 2.76-2.69 (m, 2H), 2.60-2.35 (m, 2H), 2.08 (td, J = 12.9, 4.4 Hz, 1H), 1.90 (td, J = 12.9, 4.4 Hz, 1H), 1.71 (dd, J = 73.1, 13.2 Hz, 2H). LCMS m/z 359.21 [M + 1]⁺. |
| 253 | 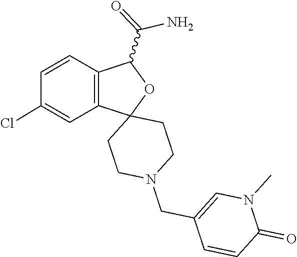 [SMILES: Cn1cc(CN2CCC3(CC2)OC(C(N)=O)c2ccc(Cl)cc23)ccc1=O] | 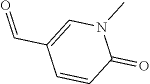 | ¹H NMR (400 MHz, DMSO-d6) δ 7.59 (d, J = 2.4 Hz, 1H), 7.50-7.27 (m, 5H), 7.14 (s, 1H), 6.37 (d, J = 9.2 Hz, 1H), 5.36 (s, 1H), 3.41 (s, 3H), 3.27 (s, 2H), 2.80-1.67 (m, 2H), 2.50-2.26 (m, 2H), 2.03 (td, J = 12.9, 4.4 Hz, 1H), 1.92-1.77 (m, 2H), 1.61 (d, J = 13.3 Hz, 1H). LCMS m/z 388.2 [M + 1]⁺. |
| 254 | 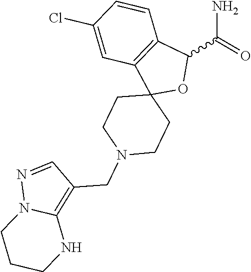 [SMILES: NC(=O)C1OC2(CCN(Cc3cnn4c3NCCC4)CC2)c2cc(Cl)ccc21] | 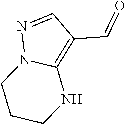 [SMILES: O=Cc1cnn2c1NCCC2] | ¹H NMR (400 MHz, DMSO-d6) δ 7.48-7.30 (m, 4H), 7.10 (s, 1H), 6.99 (s, 1H), 5.70 (s, 1H), 5.34 (s, 1H), 3.94 (t, J = 6.1 Hz, 2H), 3.24 (s, 2H), 3.16 (d, J = 6.7 Hz, 2H), 2.79-2.69 (m, 2H), 2.33 (dt, J = 49.6, 11.7 Hz, 2H), 2.09-1.93 (m, 3H), 1.89-1.74 (m, 2H), 1.59 (d, J = 13.2 Hz, 1H). LCMS m/z 402.19 [M + 1]⁺. |
| 255 | 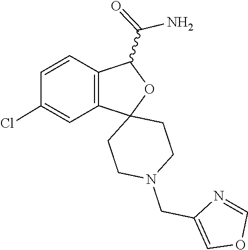 [SMILES: NC(=O)C1OC2(CCN(Cc3cocn3)CC2)c2cc(Cl)ccc21] | 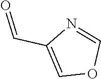 | ¹H NMR (400 MHz, DMSO-d6) δ 8.33 (s, 1H), 8.01 (s, 1H), 7.48- 7.32 (m, 4H), 7.16 (s, 1H), 5.36 (s, 1H), 3.53 (s, 2H), 2.82 (s, 2H), 2.07 (t, J = 12.3 Hz, 1H), 1.97- 1.74 (m, 2H), 1.63 (d, J = 13.6 Hz, 1H). LCMS m/z 348.23 [M + 1]⁺. |
| 256 | 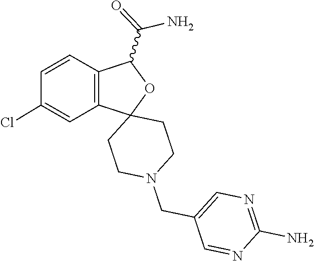 [SMILES: NC(=O)C1OC2(CCN(Cc3cnc(N)nc3)CC2)c2cc(Cl)ccc21] | 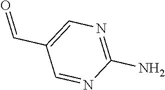 | ¹H NMR (400 MHz, DMSO-d6) δ 8.15 (s, 2H), 7.46-7.31 (m, 4H), 7.14 (s, 1H), 6.55 (s, 2H), 5.35 (s, 1H), 2.71 (d, J = 10.7 Hz, 2H), 2.48-2.26 (m, 2H), 2.09-1.99 (m, 1H), 1.90-1.75 (m, 2H), 1.61 (d, J = 13.1 Hz, 1H). 2H under water peak LCMS m/z 374.2 [M + 1]⁺. |
| 257 | 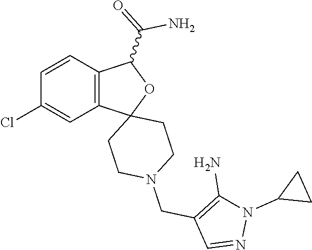 [SMILES: NC(=O)C1OC2(CCN(Cc3cnn(C4CC4)c3N)CC2)c2cc(Cl)ccc21] | 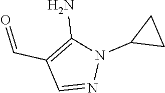 [SMILES: Nc1c(C=O)cnn1C1CC1] | ¹H NMR (400 MHz, DMSO-d6) δ 7.49-7.31 (m, 4H), 7.11 (s, 1H), 6.93 (s, 1H), 5.35 (s, 1H), 5.07 (s, 2H), 3.28 (s, 2H), 3.27-3.19 (m, 1H), 2.76 (d, J = 10.8 Hz, 2H), 2.48- 2.18 (m, 2H), 2.03 (td, J = 13.1, 3.4 Hz, 1H), 1.92- 1.73 (m, 2H), 1.60 (d, J = 13.3 Hz, 1H), 0.93- 0.88 (m, 4H). LCMS m/z 402.19 [M + 1]⁺. |
| 258 | 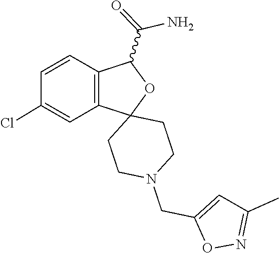 [SMILES: Cc1cc(CN2CCC3(CC2)OC(C(N)=O)c2ccc(Cl)cc23)on1] | 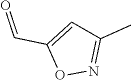 | ¹H NMR (400 MHz, DMSO-d6) δ 7.48-7.31 (m, 4H), 7.16 (s, 1H), 6.27 (s, 1H), 5.35 (s, 1H), 3.70 (s, 2H), 2.75 (d, J = 10.9 Hz, 2H), 2.62-2.34 (m, 2H), 2.22 (s, 3H), 2.07 (td, J = 12.8, 4.5 Hz, 1H), 1.88 (td, J = 12.9, 4.3 Hz, 1H), 1.84-1.76 (m, 1H), 1.61 (d, J = 13.7 Hz, 1H). LCMS m/z 362.19 [M + 1]⁺. |
| 259 | 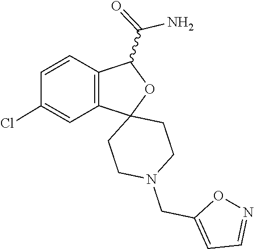 [SMILES: NC(=O)C1OC2(CCN(Cc3ccno3)CC2)c2cc(Cl)ccc21] | 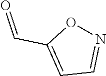 | ¹H NMR (400 MHz, DMSO-d6) δ 8.52 (d, J = 1.8 Hz, 1H), 7.48-7.41 (m, 2H), 7.40-7.33 (m, 2H), 7.16 (s, 1H), 6.43 (d, J = 1.8 Hz, 1H), 5.35 (s, 1H), 3.78 (s, 2H), 2.76 (d, J = 11.1 Hz, 2H), 2.52 (dd, J = 57.3, 11.3 Hz, 2H), 2.19-2.01 (m, 1H), 1.95-1.84 (m, 1H), 1.80 (d, J = 13.2 Hz, 1H), 1.62 (d, J = 13.4 Hz, 1H). LCMS m/z 348.23 [M + 1]⁺. |
| 260 | 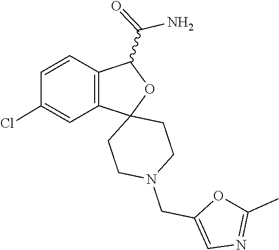 [SMILES: Cc1ncc(CN2CCC3(CC2)OC(C(N)=O)c2ccc(Cl)cc23)o1] | 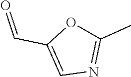 | ¹H NMR (400 MHz, DMSO-d6) δ 7.43 (t, J = 2.8 Hz, 2H), 7.41-7.31 (m, 2H), 7.15 (s, 1H), 6.90 (s, 1H), 5.35 (s, 1H), 3.57 (s, 2H), 2.74 (d, J = 11.1 Hz, 2H), 2.38 (s, 4H), 2.06 (td, J = 12.9, 4.5 Hz, 1H), 1.88 (td, J = 12.8, 4.3 Hz, 1H), 1.83- 1.76 (m, 1H), 1.61 (d, J = 13.4 Hz, 1H). LCMS m/z 362.19 [M + 1]⁺. |
| 261 | 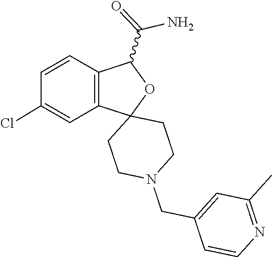 [SMILES: Cc1cc(CN2CCC3(CC2)OC(C(N)=O)c2ccc(Cl)cc23)ccn1] | 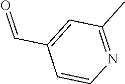 | ¹H NMR (300 MHz, DMSO-d6) δ 8.38 (d, J = 5.0 Hz, 1H), 7.50-7.30 (m, 4H), 7.22 (s, 1H), 7.16 (d, J = 6.7 Hz, 2H), 5.36 (s, 1H), 3.54 (s, 2H), 2.71 (d, J = 10.4 Hz, 3H), 2.46 (s, 3H), 2.46-2.00 (m, 3H), 1.98-1.56 (m, 2H). LCMS m/z 372.21 [M + 1]⁺. |
| 262 | 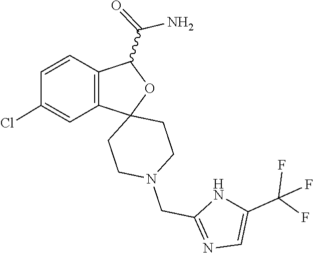 [SMILES: NC(=O)C1OC2(CCN(Cc3ncc(C(F)(F)F)[nH]3)CC2)c2cc(Cl)ccc21] | 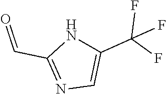 [SMILES: O=Cc1ncc(C(F)(F)F)[nH]1] | LCMS m/z 348.111 [M + 1]⁺. |
| 263 | 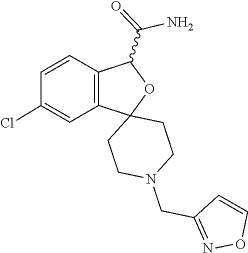 [SMILES: NC(=O)C1OC2(CCN(Cc3ccon3)CC2)c2cc(Cl)ccc21] | 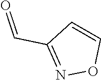 | LCMS m/z 415.113 [M + 1]⁺. |
| 264 | 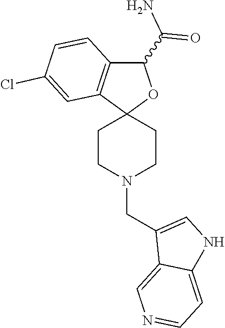 [SMILES: NC(=O)C1OC2(CCN(Cc3c[nH]c4ccncc34)CC2)c2cc(Cl)ccc21] | 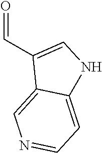 [SMILES: O=Cc1c[nH]c2ccncc12] | LCMS m/z 397.143 [M + 1]⁺. |
| 265 | 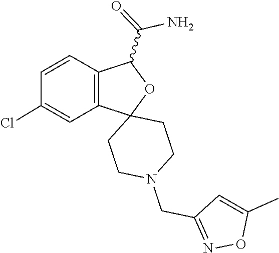 [SMILES: Cc1cc(CN2CCC3(CC2)OC(C(N)=O)c2ccc(Cl)cc23)no1] | 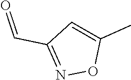 | LCMS m/z 362.126 [M + 1]⁺. |
| 266 | 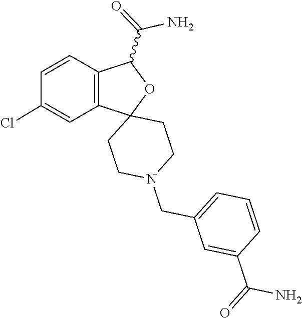 [SMILES: NC(=O)c1cccc(CN2CCC3(CC2)OC(C(N)=O)c2ccc(Cl)cc23)c1] | 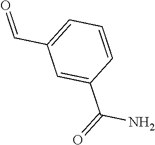 [SMILES: NC(=O)c1cccc(C=O)c1] | LCMS m/z 400.142 [M + 1]⁺. |
| 267 | 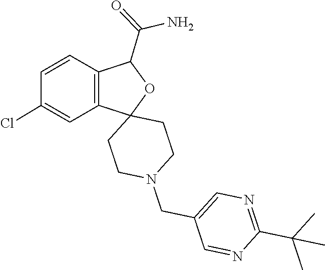 [SMILES: CC(C)(C)c1ncc(CN2CCC3(CC2)OC(C(N)=O)c2ccc(Cl)cc23)cn1] | 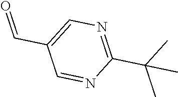 | LCMS m/z 415.189 [M + 1]⁺. |
| 268 | 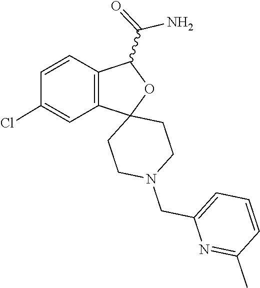 [SMILES: Cc1cccc(CN2CCC3(CC2)OC(C(N)=O)c2ccc(Cl)cc23)n1] | 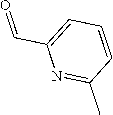 | |
| 269 | 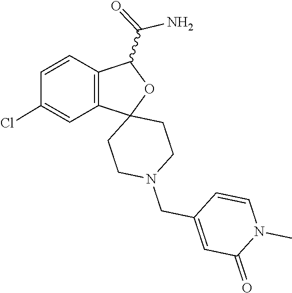 [SMILES: Cn1ccc(CN2CCC3(CC2)OC(C(N)=O)c2ccc(Cl)cc23)cc1=O] | 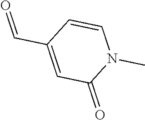 | LCMS m/z 388.142 [M + 1]⁺. |
| 270 | 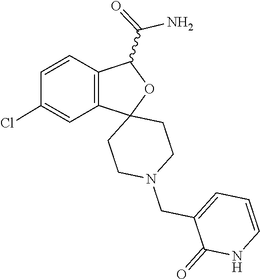 [SMILES: NC(=O)C1OC2(CCN(Cc3ccc[nH]c3=O)CC2)c2cc(Cl)ccc21] | 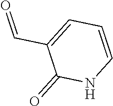 | LCMS m/z 374.126 [M + 1]⁺. |
| 271 | 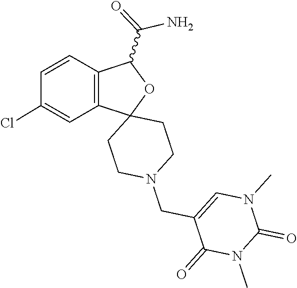 [SMILES: Cn1cc(CN2CCC3(CC2)OC(C(N)=O)c2ccc(Cl)cc23)c(=O)n(C)c1=O] | 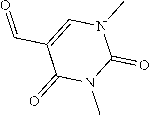 | |
| 272 | 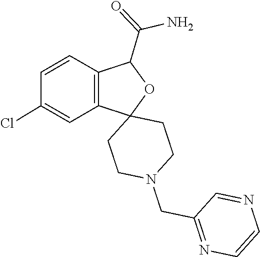 [SMILES: NC(=O)C1OC2(CCN(Cc3cnccn3)CC2)c2cc(Cl)ccc21] | 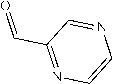 | LCMS m/z 359.127 [M + 1]⁺. |
| 273 | 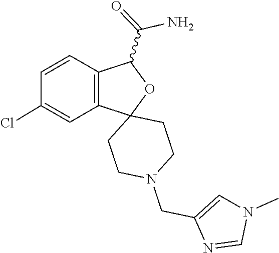 [SMILES: Cn1cnc(CN2CCC3(CC2)OC(C(N)=O)c2ccc(Cl)cc23)c1] | 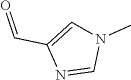 | |
| 274 | 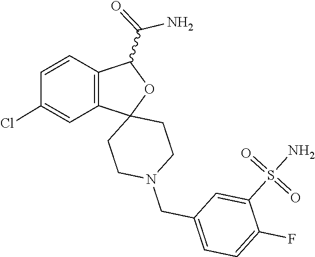 [SMILES: NC(=O)C1OC2(CCN(Cc3ccc(F)c(S(N)(=O)=O)c3)CC2)c2cc(Cl)ccc21] | 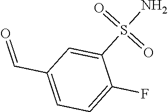 [SMILES: NS(=O)(=O)c1cc(C=O)ccc1F] | LCMS m/z 454.099 [M + 1]⁺. |
| 275 | 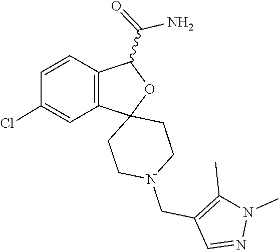 [SMILES: Cc1c(CN2CCC3(CC2)OC(C(N)=O)c2ccc(Cl)cc23)cnn1C] | 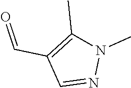 | |
| 276 | 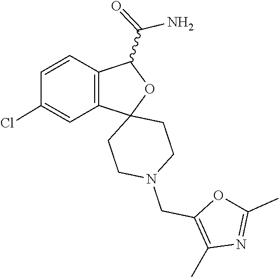 [SMILES: Cc1nc(C)c(CN2CCC3(CC2)OC(C(N)=O)c2ccc(Cl)cc23)o1] | 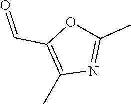 | LCMS m/z 376.142 [M + 1]⁺. |

### Compound 277

### 5-chloro-1′-[[1-[(2S)-2,3-dihydroxy-3-methyl-butyl]pyrazol-4-yl]methyl]spiro[1H-isobenzofuran-3,4′-piperidine]-1-carboxamide (277)

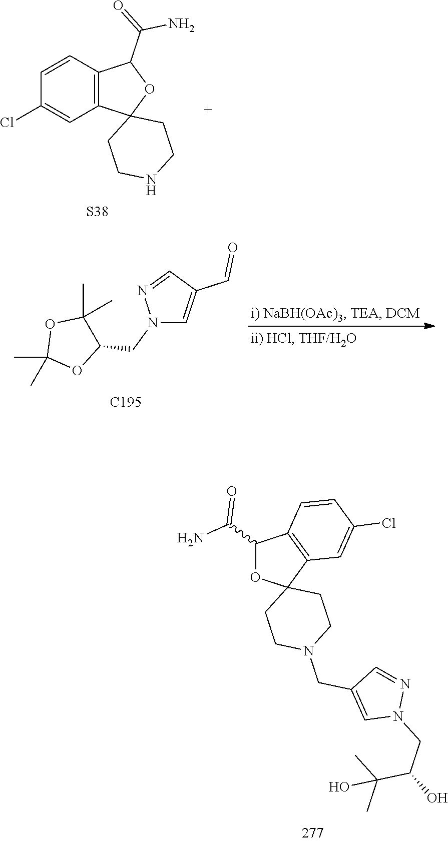 [SMILES: NC(=O)C1OC2(CCNCC2)c2cc(Cl)ccc21 ; "S38" ; CC1(C)O[C@@H](Cn2cc(C=O)cn2)C(C)(C)O1 ; "C195" ; CC(C)(O)[C@@H](O)Cn1cc(CN2CCC3(CC2)OC(C(N)=O)c2ccc(Cl)cc23)cn1]

To a vial containing 5-chlorospiro[1H-isobenzofuran-3,4′-piperidine]-1-carboxamide (Trifluoroacetate salt) (107 mg, 0.2445 mmol) in DCM (1.9 mL) and TEA (70 μL, 0.5022 mmol) was added 1-[[(4S)-2,2,5,5-tetramethyl-1,3-dioxolan-4-yl]methyl]pyrazole-4-carbaldehyde (90 mg, 0.3777 mmol). To the resulting mixture was added triacetoxyboranuide (Sodium salt) (185 mg, 0.8729 mmol) and stirred for 2 days. Reaction was quenched into sat. aq. NaHCO₃ and extracted with DCM (3×). The pooled organics were passed through a phase separator and concentrated. The resulting crude residue was diluted with THF (5 mL) and hydrogen chloride (500 μL of 4 M in dioxanes, 2.000 mmol) was added. The reaction was stirred overnight and then water (500 μL, 27.75 mmol) was added and the reaction was stirred overnight again. The reaction was then concentrated under reduced pressure. The residue was purified by reversed-phase HPLC (Method: Waters XBridge Prep C8 Column; 30×150 mm, 5 micron. Gradient: Acetonitrile in Water with 10 mM Ammonium Hydroxide.) to yield 5-chloro-1′-[[1-[(2S)-2,3-dihydroxy-3-methyl-butyl]pyrazol-4-yl]methyl]spiro[1H-isobenzofuran-3,4′-piperidine]-1-carboxamide 277 (21.7 mg, 18%) ¹H NMR (400 MHz, DMSO-d6) δ 7.57 (s, 1H), 7.49-7.29 (m, 5H), 7.12 (s, 1H), 5.35 (s, 1H), 4.92 (d, J=6.1 Hz, 1H), 4.46 (s, 1H), 4.33 (dd, J=13.7, 1.9 Hz, 1H), 3.90-3.79 (m, 1H), 3.52 (t, J=7.7 Hz, 1H), 3.39 (s, 2H), 3.31 (s, 1H), 2.83-2.72 (m, 2H), 2.46-2.20 (m, 1H), 2.03 (t, J=11.3 Hz, 1H), 1.91-1.75 (m, 2H), 1.60 (d, J=13.1 Hz, 1H), 1.08 (d, J=21.7 Hz, 6H).). LCMS m/z 449.28 [M+1]⁺.

### Compound 278

### 5-chloro-1′-[[1-[(2R)-2,3-dihydroxy-3-methyl-butyl]pyrazol-4-yl]methyl]spiro[1H-isobenzofuran-3,4′-piperidine]-1-carboxamide (278)

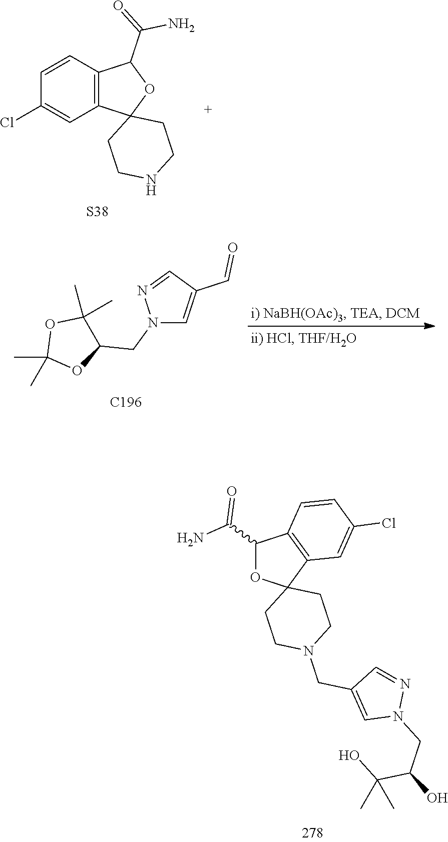 [SMILES: NC(=O)C1OC2(CCNCC2)c2cc(Cl)ccc21 ; "S38" ; CC1(C)O[C@H](Cn2cc(C=O)cn2)C(C)(C)O1 ; "C196" ; CC(C)(O)[C@H](O)Cn1cc(CN2CCC3(CC2)OC(C(N)=O)c2ccc(Cl)cc23)cn1]

5-chloro-1′-[[1-[(2R)-2,3-dihydroxy-3-methyl-butyl]pyrazol-4-yl]methyl]spiro[1H-isobenzofuran-3,4′-piperidine]-1-carboxamide (278) was prepared using the same method as used to prepare compound 277 with starting aldehyde C196.

¹H NMR (400 MHz, DMSO-d6) δ 7.57 (s, 1H), 7.48-7.28 (m, 5H), 7.12 (s, 1H), 5.35 (s, 1H), 4.92 (d, J=6.1 Hz, 1H), 4.46 (s, 1H), 4.38-4.29 (m, 1H), 3.85 (dd, J=13.8, 9.6 Hz, 1H), 3.52 (t, J=7.6 Hz, 1H), 3.40 (s, 2H), 2.82-2.74 (m, 2H), 2.47-2.20 (m, 2H), 2.04 (t, J=14.9 Hz, 1H), 1.94-1.55 (m, 3H), 1.09 (d, J=21.7 Hz, 6H). LCMS m/z 449.33 [M+1]⁺.

### Preparation S39

### 1-[3-[tert-butyl(dimethyl)silyl]oxy-2-[[tert-butyl(dimethyl)silyl]oxymethyl]-2-methyl-propyl]pyrazole-4-carbaldehyde (S39)

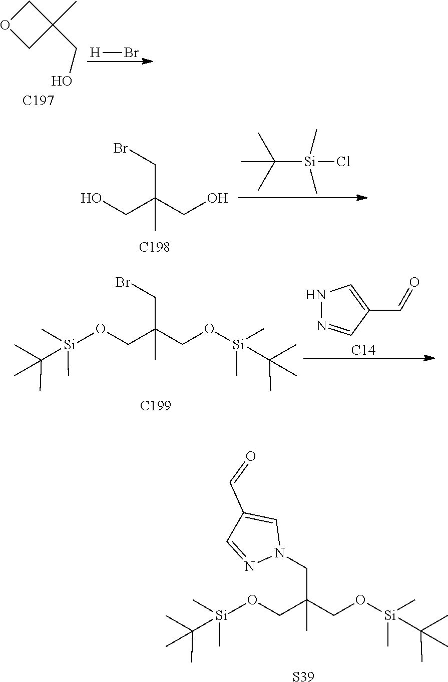

### Step 1: 2-(bromomethyl)-2-methyl-propane-1, 3-diol (C198)

To a mixture of (3-methyloxetan-3-yl)methanol C197 (10 mL, 100.3 mmol) in THF (70 mL) at 0° C. was added hydrogen bromide (14 mL of 48% w/w, 123.7 mmol). After stirring for 24 hours, the mixture was concentrated to a minimum volume, diluted in DCM/Methanol and the excess HBr was quenched with sat. sodium bicarbonate. The layers were split and the organic layer was dried with sodium sulfate, filtered, rinsed with methanol and concentrated to yield 2-(bromomethyl)-2-methyl-propane-1,3-diol C198 (13.6682 g, 74%) ¹H NMR (400 MHz, Chloroform-d) δ 3.47 (d, J=1.1 Hz, 6H), 0.96 (s, 3H).

### Step 2:[2-(bromomethyl)-3-[tert-butyl(dimethyl)silyl]oxy-2-methyl-propoxy]-tert-butyl-dimethyl-silane (C199)

To a mixture of 2-(bromomethyl)-2-methyl-propane-1,3-diol C198 (10 g, 54.09 mmol) in DCM (200 mL) was added imidazole (7.7 g, 113.1 mmol) followed by TBSCl (17 g, 112.8 mmol). After 5 min the mixture had precipitated a white crystalline solid. The mixture was filtered, rinsed with DCM, and concentrated. The mixture was diluted with heptane (25 mL) to further precipitate imidazole/imidazole HCl, filtered, and the solid was rinsed with additional heptane (10 mL). The mixture was concentrated, which precipitated additional solid. The mixture was diluted and concentrated twice more with heptane (50 mL) to afford [2-(bromomethyl)-3-[tert-butyl(dimethyl)silyl]oxy-2-methyl-propoxy]-tert-butyl-dimethyl-silane C199 (22246 mg, 100%) ¹H NMR (400 MHz, Chloroform-d) δ 3.44 (s, 4H), 3.40 (s, 2H), 0.94 (s, 3H), 0.89 (s, 18H), 0.04 (d, J=1.2 Hz, 12H).

### Step 3: 1-[3-[tert-butyl(dimethyl)silyl]oxy-2-[[tert-butyl(dimethyl)silyl]oxymethyl]-2-methyl-propyl]pyrazole-4-carbaldehyde (S39)

To a vial was added 1H-pyrazole-4-carbaldehyde (2 g, 20.81 mmol), dipotassium; carbonate (4 g, 28.94 mmol), and [2-(bromomethyl)-3-[tert-butyl(dimethyl)silyl]oxy-2-methyl-propoxy]-tert-butyl-dimethyl-silane C199 (9.5 g, 23.08 mmol) in DMF (20 mL). The mixture was heated to 130° C. After 3 hours the mixture was cooled to room temperature, diluted with water (100 mL) and heptane (100 mL). The layers were mixed, and the aqueous layer was washed with heptane (2×100 mL). The combined organic layer was washed with water (100 mL), brine (100 mL) and the organic layer was dried with sodium sulfate and concentrated. Purification by silica gel chromatography (Gradient: 0-60% EtOAc:Heptane) yielded the product 1-[3-[tert-butyl(dimethyl)silyl]oxy-2-[[tert-butyl(dimethyl)silyl]oxymethyl]-2-methyl-propyl]pyrazole-4-carbaldehyde S39 (2390 mg, 23%) ¹H NMR (400 MHz, Chloroform-d) δ 9.85 (s, 1H), 7.98-7.91 (m, 2H), 4.12 (s, 2H), 3.43-3.29 (m, 4H), 0.91 (s, 18H), 0.84 (s, 3H), 0.05 (d, J=0.6 Hz, 12H). LCMS m/z 427.31 [M+H]⁺.

### Compound 279

### 5-chloro-1′-[[1-[3-hydroxy-2-(hydroxymethyl)-2-methyl-propyl]pyrazol-4-yl]methyl]spiro[1H-isobenzofuran-3,4′-piperidine]-1-carboxamide (279)

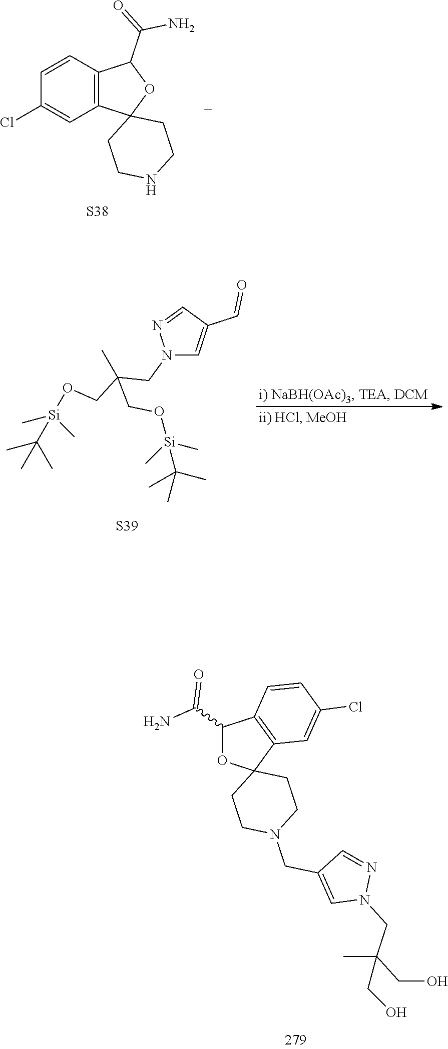 [SMILES: NC(=O)C1OC2(CCNCC2)c2cc(Cl)ccc21 ; "S38" ; CC(CO[Si](C)(C)C(C)(C)C)(CO[Si](C)(C)C(C)(C)C)Cn1cc(C=O)cn1 ; "S39" ; CC(CO)(CO)Cn1cc(CN2CCC3(CC2)OC(C(N)=O)c2ccc(Cl)cc23)cn1]

5-chloro-1′-[[1-[3-hydroxy-2-(hydroxymethyl)-2-methyl-propyl]pyrazol-4-yl]methyl]spiro[1H-isobenzofuran-3,4′-piperidine]-1-carboxamide (279) was prepared using the same method as used to prepare compound 278 with starting aldehyde S39 with the exception of the use of MeOH instead of THF/H₂O used as the solvent during the reaction with HCl.

¹H NMR (400 MHz, DMSO-d6) δ 7.55 (s, 1H), 7.49-7.29 (m, 5H), 7.12 (s, 1H), 5.34 (s, 1H), 4.58 (t, J=5.3 Hz, 2H), 3.98 (s, 2H), 3.27-3.09 (m, 4H), 2.86-2.68 (m, 2H), 2.48-2.22 (m, 2H), 2.15-1.96 (m, 1H), 1.93-1.55 (m, 3H), 0.68 (s, 3H). LCMS m/z 449.33 [M+1]⁺.

### Compound 280

### 5-chloro-1′-[[1-[2-hydroxy-2-(1-hydroxycyclobutyl)ethyl]pyrazol-4-yl]methyl]spiro[1H-isobenzofuran-3,4′-piperidine]-1-carboxamide (280)

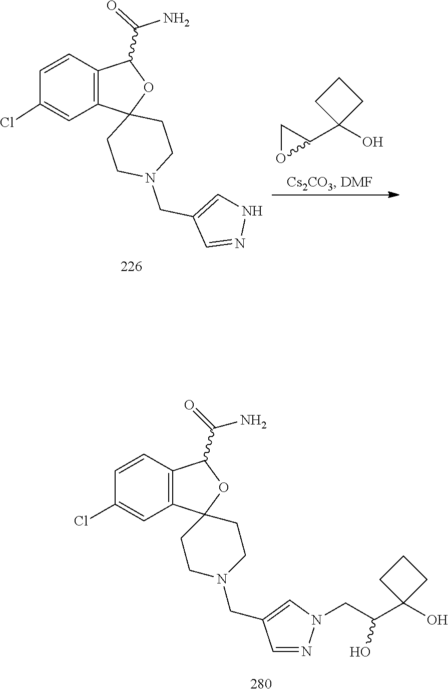 [SMILES: NC(=O)C1OC2(CCN(Cc3cn[nH]c3)CC2)c2cc(Cl)ccc21 ; OC1(C2CO2)CCC1 ; NC(=O)C1OC2(CCN(Cc3cnn(CC(O)C4(O)CCC4)c3)CC2)c2cc(Cl)ccc21]

To a suspension of 5-chloro-1′-(1H-pyrazol-4-ylmethyl)spiro[1H-isobenzofuran-3,4′-piperidine]-1-carboxamide (30 mg, 0.08650 mmol) (Compound 226), Cs₂CO₃ (114 mg, 0.3499 mmol) in DMF (1.0 mL) was added 1-(oxiran-2-yl)cyclobutanol (11 mg, 0.09637 mmol) and stirred at 75° C. for 5 hours. The reaction was filtered through a PTFE 0.45 um syringe filter, and concentrated. The crude residue was purified by reverse phase HPLC (Column:C18. Gradient: 0-100% MeCN in aqueous solution of 10 mM Ammonium Bicarbonate) to provide 5-chloro-1′-[[1-[2-hydroxy-2-(1-hydroxycyclobutyl)ethyl]pyrazol-4-yl]methyl]spiro[1H-isobenzofuran-3,4′-piperidine]-1-carboxamide 280 (17.8 mg, 41%). ¹H NMR (400 MHz, DMSO-d6) δ 8.47-7.29 (m, 5H), 7.57 (s, 1H), 7.12 (s, 1H), 5.34 (s, 1H), 5.04 (s, 1H), 4.89 (d, J=6.7 Hz, 1H), 4.21-4.11 (m, 1H), 3.99-3.86 (m, 1H), 3.70 (t, J=8.9 Hz, 1H), 3.39 (s, 2H), 2.81-2.72 (m, 2H), 2.43-2.17 (m, 2H), 2.05-1.41 (m, 8H). ESI-MS m/z 461.29 [M+1]⁺.

### Compounds 281-285

Compounds 281-285 (see Table 18) were prepared from Compound 226 and the appropriate electrophilic reagents employing the same method as compound 280. Any modifications to methods are noted in Table 18 and accompanying footnotes.

TABLE 18
Structure and physicochemical data for compounds 281-285

| | | | LCMS m/z |
|---|---|---|---|
| Compound | Product | Electrophile | [M + H]⁺ |
| 281 | 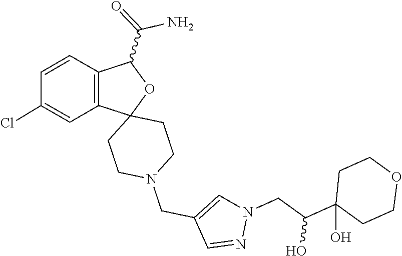 [SMILES: NC(=O)C1OC2(CCN(Cc3cnn(CC(O)C4(O)CCOCC4)c3)CC2)c2cc(Cl)ccc21] | 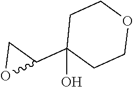 | ¹H NMR (300 MHz, DMSO-d6) δ 7.57 (s, 1H), 7.38 (dt, J = 18.9, 5.6 Hz, 5H), 7.11 (s, 1H), 5.35 (s, 1H), 5.02 (s, 1H), 4.35 (d, J = 13.7 Hz, 1H), 3.90 (dd, J = 13.9, 9.6 Hz, 1H), 3.71- 3.46 (m, 5H), 3.39 (s, 2H), 2.83-2.67 (m, 2H), 2.46- 2.19 (m, 4H), 2.11- 1.95 (m, 1H), |
| | | | 1.93-1.52 (m, |
| | | | 4H), 1.41-1.13 |
| | | | (m, 2H). LCMS |
| | | | m/z 491.32 |
| | | | [M + 1]+. |
| 282 | 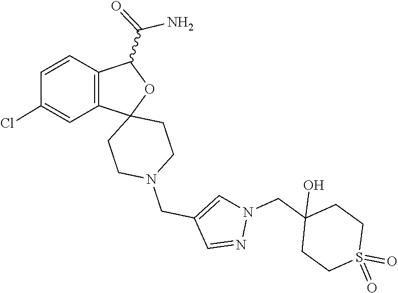 [SMILES: NC(=O)C1OC2(CCN(Cc3cnn(CC4(O)CCS(=O)(=O)CC4)c3)CC2)c2cc(Cl)ccc21] | 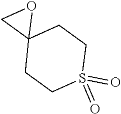 | LCMS m/z 509.2 [M + 1]+. |
| 283 | 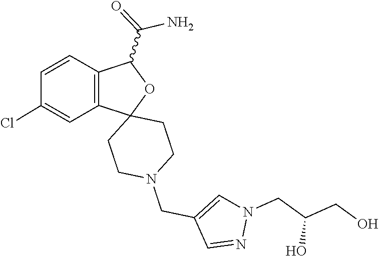 [SMILES: NC(=O)C1OC2(CCN(Cc3cnn(C[C@@H](O)CO)c3)CC2)c2cc(Cl)ccc21] | 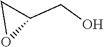 | ¹H NMR (400 MHz, DMSO-d6) δ 7.56 (s, 1H), 7.48-7.30 (m, 5H), 7.13 (s, 1H), 5.35 (s, 1H), 4.95 (s, 1H), 4.72 (s, 1H), 4.17 (dd, J = 13.7, 4.1 Hz, 1H), 3.94 (dd, J = 13.8, 7.5 Hz, 1H), 3.78 (t, J = 6.2 Hz, 1H), 3.41 (s, 2H), 3.28 (td, J = 10.8, 5.5 Hz, 1H), 2.77 (d, J = 10.7 Hz, |
| | | | 2H), 2.47-2.25 |
| | | | (m, 2H), 2.04 (td, J = |
| | | | 12.9, 4.3 Hz, |
| | | | 1H), 1.90-1.74 |
| | | | (m, 2H), 1.61 (d, J = |
| | | | 13.3 Hz, 1H). |
| | | | LCMS m/z 421.14 |
| | | | [M + 1]+. |
| 284 | 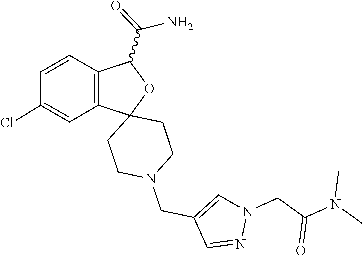 [SMILES: CN(C)C(=O)Cn1cc(CN2CCC3(CC2)OC(C(N)=O)c2ccc(Cl)cc23)cn1] | 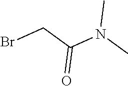 | ¹H NMR (400 MHz, DMSO-d6) δ 7.53 (s, 1H), 7.48-7.28 (m, 5H), 7.13 (s, 1H), 5.35 (s, 1H), 5.04 (s, 2H), 3.44 (s, 2H), 3.01 (s, 3H), 2.84 (s, 3H), 2.77 (d, J = 12.6 Hz, 2H), 2.48- 2.24 (m, 2H), 2.04 (td, J = 12.7, 4.4 Hz, 1H), 1.92- 1.75 (m, 2H), 1.61 (d, J = 13.2 Hz, |
| | | | 1H). LCMS m/z |
| | | | 432.31 [M + 1]+. |
| 285 | 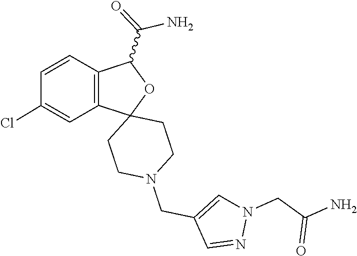 [SMILES: NC(=O)Cn1cc(CN2CCC3(CC2)OC(C(N)=O)c2ccc(Cl)cc23)cn1] | 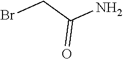 | LCMS m/z 404.27 [M + 1]+. |

### Compound 286

### 5-methyl-1′-[(1-methylpyrazol-4-yl)methyl]spiro[1H-isobenzofuran-3,4′-piperidine](286)

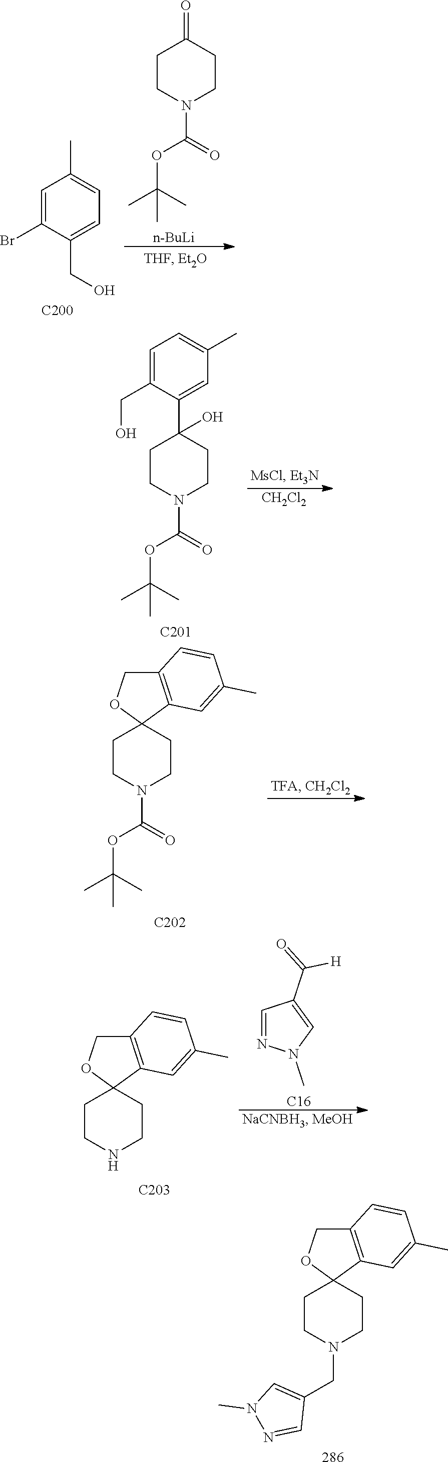

### Step 1: Preparation of tert-butyl 4-hydroxy-4-[2-(hydroxymethyl)-5-methyl-phenyl]piperidine-1-carboxylate (C201)

To a stirred solution of (2-bromo-4-methyl-phenyl)methanol (300 mg, 0.0013 mol) in THF (6 mL) was added n-BuLi (1.2800 mL of 2.5 M, 0.0032 mol) at −78° C., and solution stirred for 10 minutes. To this was added a solution of tert-butyl 4-oxopiperidine-1-carboxylate (278.95 mg, 0.0014 mol) in diethyl ether (2 mL) at −78° C. The reaction mixture was stirred for 2 hours at −78° C. The reaction mixture temperature allowed to warm to ambient temperature, quenched with saturated NH₄Cl solution, and extracted with EtOAc (2×50 mL). The combined organic layers were washed with brine, dried over Na₂SO₄, and concentrated to afford tert-butyl 4-hydroxy-4-[2-(hydroxymethyl)-5-methyl-phenyl]piperidine-1-carboxylate (550 mg, 45%). ESI-MS m/z: 322.23 [M+1]⁺

### Step 2: Preparation of tert-butyl 5-methylspiro[1H-isobenzofuran-3,4′-piperidine]-1′-carboxylate (C202)

To a stirred solution of tert-butyl 4-hydroxy-4-[2-(hydroxymethyl)-5-methyl-phenyl]piperidine-1-carboxylate (370 mg, 1.1512 mmol) in DCM (10 mL), Et₃N (582.45 mg, 0.8023 mL, 5.7560 mmol) was added at 0° C., followed by the addition of MsCl (197.81 mg, 0.1337 mL, 1.7268 mmol). The reaction mixture was stirred at ambient temperature for 4 hours. The reaction mixture was diluted with DCM (30 mL), and washed 3 times with water (10 mL). The solvent was evaporated, and crude residue purified by normal phase flash column chromatography (Gradient: 10-20% EtOAc/Hexanes) to provide tert-butyl 5-methylspiro[1H-isobenzofuran-3,4′-piperidine]-1′-carboxylate (150 mg, 43%). ¹H NMR (DMSO-d6): δ 7.15 (d, J=8 Hz, 1H), 7.09 (d, J=4.24, 2H), 4.94 (s, 2H), 3.93 (brs, 2H), 3.05 (brs, 2H), 2.30 (s, 3H), 1.78-1.72 (m, 2H), 1.57 (d, J=12.4 Hz, 2H), 1.42 (s, 9H). ESI-MS m/z: 304.0 [M+1]⁺.

### Step 3: Preparation of 5-methylspiro[1H-isobenzofuran-3,4′-piperidine](C203)

To a stirred solution of tert-butyl 5-methylspiro[1H-isobenzofuran-3,4′-piperidine]-1′-carboxylate (150 mg, 0.4944 mmol) in DCM (5 mL), TFA (281.86 mg, 0.1904 mL, 2.4720 mmol) was added at 0° C. and stirred at ambient temperature for 1 hour. The reaction mixture was diluted with DCM (30 mL) and washed 3 times with saturated sodium carbonate solution (10 mL) and water (10 mL). The solvent was evaporated to provide 5-methylspiro[1H-isobenzofuran-3,4′-piperidine](95 mg, 95%) which used in next step without further purification. ESI-MS m/z: 204.0 [M+1]⁺.

### Step 4: Preparation of 5-methyl-1′-[(1-methylpyrazol-4-yl)methyl]spiro[1H-isobenzofuran-3,4′-piperidine](286)

To a stirred solution of 5-methylspiro[1H-isobenzofuran-3,4′-piperidine](90 mg, 0.4067 mmol) in methanol (2 mL) was added 1-Methyl-1H-pyrazole-4-carbaldehyde (67.170 mg, 0.6100 mmol). The mixture was stirred for 30 minutes, followed by addition of NaCNBH₃ (76.674 mg, 1.2201 mmol) at 0° C. The reaction mixture was stirred at ambient temperature for 6 hours. The reaction mixture was quenched with ice, and then the excess methanol was evaporated. The crude residue was diluted with DCM (50 mL), and washed 3 times with water (20 mL). The excess solvent was evaporated and crude residue purified by reverse phase column chromatography (Column: YMC Triart Actus C18 (250×20 mm, 5μ); Gradient: 30-95% MeCN/20 mM Ammonium Bicarbonate in water) to provide 5-methyl-1′-[(1-methylpyrazol-4-yl)methyl]spiro[1H-isobenzofuran-3,4′-piperidine]286 (45 mg, 36%) ¹H NMR (DMSO-d6): δ 7.56 (s, 1H), 7.30 (s, 1H), 7.12 (d, J=7.6 Hz, 1H), 7.06 (d, J=7.68 Hz, 1H), 7.02 (s, 2H), 4.88 (s, 2H), 3.79 (s, 3H), 3.37 (s, 3H), 2.70 (d, J=10.84 Hz, 2H), 2.30 (s, 3H), 2.27-2.21 (m, 2H), 1.87-1.79 (m, 2H), 1.56 (d, J=12.64 Hz, 2H). ESI-MS m/z: 298.0 [M+1]⁺.

### Compound 287

### 5-chloro-1,1-dimethyl-1′-[[1-(2-methylsulfonylethyl)pyrazol-4-yl]methyl]spiro[isobenzofuran-3,4′-piperidine](287)

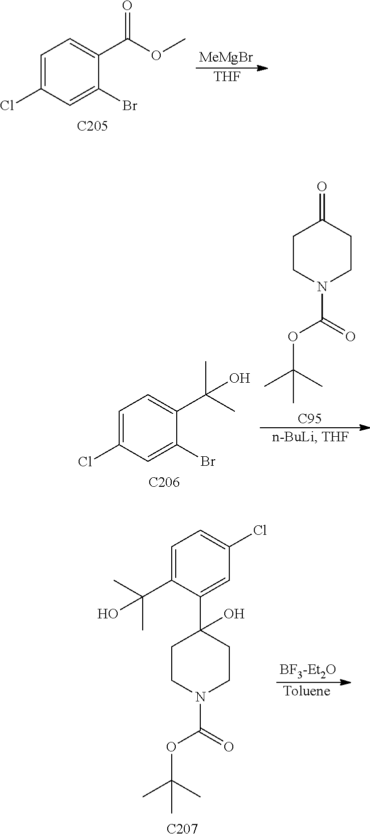

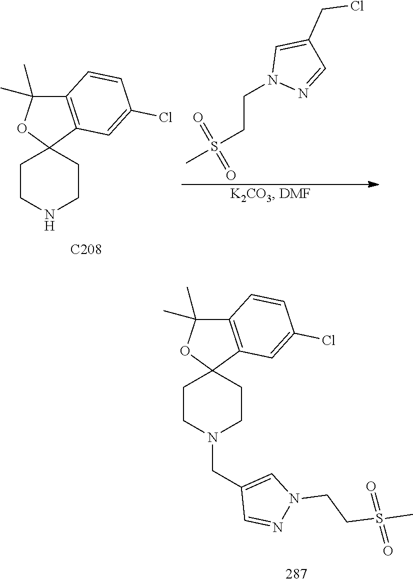

### Step 1: Preparation of 2-(2-bromo-4-chloro-phenyl)propan-2-ol (C206)

To a stirred solution of methyl 2-bromo-4-chloro-benzoate (10 g, 40.082 mmol) in THF (200 mL) cooled to 0° C., MeMgBr (19.118 g, 160.33 mmol) was slowly added while maintaining temperature at 0° C. The reaction mixture was quenched with saturated ammonium chloride solution, (200 mL) and extracted with EtOAc (3×300 ml). The organic layer was washed with brine (400 mL), and dried over anhydrous Na₂SO₄. The organic layer was filtered and concentrated to afford 2-(2-bromo-4-chloro-phenyl)propan-2-ol (10 g, 100%) ¹H NMR (CDCl₃, 400 MHz): δ=7.65-7.63 (d, 1H, J=8.8 Hz), 7.598-7.592 (d, 1H, J=2.4 Hz), 2.55 (m, 1H), 1.69-1.73 ppm (m, 6H).

### Step 2: Preparation of tert-butyl 4-[5-chloro-2-(I-hydroxy-1-methyl-ethyl)phenyl]-4-hydroxy-piperidine-1-carboxylate (C207)

To a stirred solution of 2-(2-bromo-4-chloro-phenyl)propan-2-ol (2.5 g, 0.0089 mol) in THF (37.5 mL) was added n-BuLi (2.0 g, 3 mL, 0.0318 mol) at −78° C. The reaction mixture was stirred for 30 min at the same temperature, and then tert-butyl 4-oxopiperidine-1-carboxylate (2.2 g, 0.0110 mol) in THF (5 mL) was added. The reaction mixture was allowed to warm to room temperature, then stirring was continued for 3 hours. The reaction mixture was quenched with saturated ammonium chloride solution (200 ml), then extracted with EtOAc (3×100 ml). The combined organic layer was dried over Na₂SO₄ and concentrated to obtained tert-butyl 4-[5-chloro-2-(1-hydroxy-1-methyl-ethyl)phenyl]-4-hydroxy-piperidine-1-carboxylate (5 g, 21%), which was carried forward without further purification.

### Step 3: Preparation of 5-chloro-1,1-dimethyl-spiro[isobenzofuran-3,4′-piperidine](C208)

To a stirred solution of tert-butyl 4-[5-chloro-2-(1-hydroxy-1-methyl-ethyl)phenyl]-4-hydroxy-piperidine-1-carboxylate (1.5 g, 0.7300 mmol) in toluene (15 mL), was added BF₃·OEt₂ (2.8779 g, 2.5468 mL, 20.277 mmol) slowly at room temperature. The reaction was stirred for 16 hours at room temperature. The reaction mixture was concentrated and neutralized with sodium bicarbonate solution, then added to water (100 ml), and extracted with EtOAc (2×100 ml). The combined organic layer was concentrated to provide 5-chloro-1,1-dimethyl-spiro[isobenzofuran-3,4′-piperidine](190 mg, 103%) as a crude intermediate without further purification, which was directly carried forward to the next step. ESI-MS m/z: 252.0 [M+1]⁺.

### Step 4: Preparation of 5-chloro-1,1-dimethyl-1′-[[1-(2-methylsulfonylethyl)pyrazol-4-yl]methyl]spiro[isobenzofuran-3,4′-piperidine](287)

To a stirred solution of 5-chloro-1,1-dimethyl-spiro[isobenzofuran-3,4′-piperidine](100 mg, 293.94 μmol) and 4-(chloromethyl)-1-(2-methylsulfonylethyl)pyrazole (82 mg, 294.58 μmol) in MeCN (2.0 mL) and DMF (1.0 mL), were added K₂CO₃ (207 mg, 0.0015 mol) followed by KI (10 mg, 60.24 μmol) at ambient temperature. The reaction mixture was then stirred at room temperature for 16 hours. The reaction mixture was filtered through celite pad and washed with EtOAc (2×200 mL). The filtrates were concentrated to provide a crude residue that was purified by reverse phase HPLC (Column: -xselectPhenyl hexyle (150×25)mm, 5u; Gradient: 2-25% MeCN:MeOH (1:1) in 0.1% aqueous TFA) to provide 5-chloro-1,1-dimethyl-1′-[[1-(2-methylsulfonylethyl)pyrazol-4-yl]methyl]spiro[isobenzofuran-3,4′-piperidine](287) (23 mg, 14%) ¹H NMR (400 MHz, DMSO-d6) δ 8.00 (d, J=12.0 Hz, 1H), 7.67 (d, J=8.0 Hz, 1H), 7.42 (dd, J=8.0 Hz, 1.6 Hz, 1H), 7.36 (d, J=4.8 Hz, 1H), 7.33-6.52 (m, 1H), 4.59 (t, J=6.4 Hz, 2H), 4.41 (d, J=6.0 Hz, 2H), 3.70 (t, J=6.8 Hz, 2H), 3.37 (s, 2H), 3.12 (s, 2H) 2.89 (s, 3H), 2.17-2.07 (m, 2H), 1.81 (d, J=13.6 Hz, 2H), 1.46 (s, 6H). ESI-MS m/z: 438.0 [M+1]⁺.

### Compound 288

### 2-methyl-1-[[5-[(1,1,5-trimethylspiro[isobenzofuran-3,4′-piperidine]-1′-yl)methyl]-2-pyridyl]oxy]propan-2-amine (288)

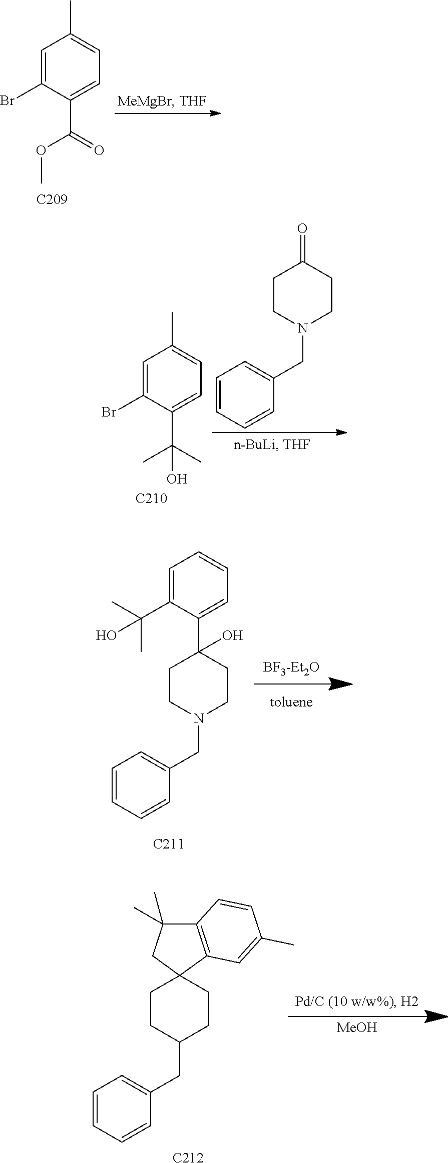

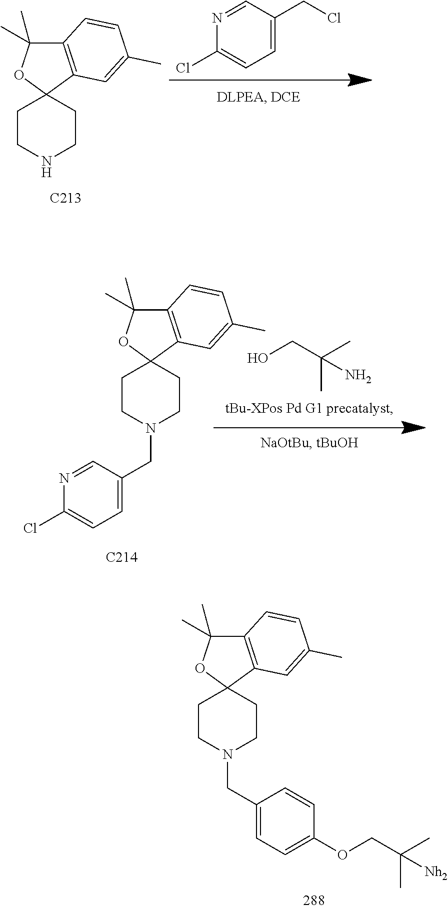

### Step 1: Preparation of 2-(2-bromo-4-methyl-phenyl)propan-2-ol (C210)

To a stirred solution of methyl 2-bromo-4-methyl-benzoate (5.0 g, 21.83 mmol) in THF (50 mL), was added MeMgBr (21.83 mL of 3 M, 65.481 mmol) slowly at ambient temperature, then the resulting reaction mixture was stirred for 16 hours. Reaction mixture was quenched with ammonium chloride solution (200 ml), extracted with ethyl acetate (200 ml), and organic layer dried with sodium sulfate. Solution was concentrated to provide 2-(2-bromo-4-methyl-phenyl)propan-2-ol (5.5 g, 93%). ¹H NMR (400 MHz, CDCl₃), δ ppm 7.51 (d, J=8.4 MHz, 1H), 7.41 (s, 1H), 7.09 (d, J=6.8 MHz, 1H), 2.29 (s, 3H), 1.72 (s, 6H).

### Step 2: Preparation of 1-benzyl-4-[2-(1-hydroxy-1-methyl-ethyl)-5-methyl-phenyl]piperidin-4-ol (C211)

To a stirred solution of 2-(2-bromo-4-methyl-phenyl)propan-2-ol (5.5 g, 24.0 mmol) in THF (45 mL), was added n-BuLi (22.1 mL of 2.5 M, 55.2 mmol) slowly over 15 min at −78° C., and the mixture was stirred for 1 hour at the same temperature. To the mixture was slowly added a 1-benzylpiperidin-4-one (6.36 g, 33.6 mmol) solution in tetrahydrofuran (10 mL) at −78° C. for 10 minutes. This resulting mixture was slowly warmed up to ambient temperature and stirred for 18 hours. The reaction mixture was quenched by the addition of ammonium chloride solution (100 ml), extracted with ethyl acetate (200 ml), and the organic layer dried with sodium sulfate then concentrated. The crude residue was purified by flash column chromatography (Gradient: 5% methanol in DCM) to afford 1-benzyl-4-[2-(1-hydroxy-1-methyl-ethyl)-5-methyl-phenyl]piperidin-4-ol (3.2 g, 32%) ¹H NMR (400 MHz, CDCl₃), δ ppm 7.63 (s, 1H), 7.43-7.32 (m, 5H), 7.16 (d, J=7.6 Hz, 1H), 6.97 (d, J=7.6 Hz, 1H), 4.07 (s, 2H), 3.21 (br, s, 4H), 2.87 (br, s, 2H), 2.30 (s, 3H), 2.69-2.03 (m, 2H), 1.76 (s, 6H), 1.33-1.30 (m, 1H), 0.91-0.88 (m, 1H). ESI-MS m/z 340.0 [M+1]⁺.

### Step 3: Preparation of 1′-benzyl-1,1,5-trimethyl-spiro[isobenzofuran-3,4′-piperidine](C212)

To a stirred solution of 1-benzyl-4-[2-(1-hydroxy-1-methyl-ethyl)-5-methyl-phenyl]piperidin-4-ol (3.2 g, 9.43 mmol) in toluene (64 mL) was added boron trifluoride diethyl etherate (30.1 g, 26.6 mL, 212.1 mmol), and the reaction mixture was stirred for 16 hours. The solvent was evaporated under reduced pressure to obtain a crud residue that was quenched with water (200 mL), extracted with ethyl acetate (3×100 mL), dried over sodium sulfate, and concentrated. The crude residue was purified by flash column chromatography (Gradient: 30-40% of ethyl acetate in hexanes) to provide 1′-benzyl-1,1,5-trimethyl-spiro[isobenzofuran-3,4′-piperidine](2.9 g, 78%) ¹H NMR (400 MHz, CDCl₃), δ ppm 7.48-7.43 (m, 5H), 7.12 (d, J=8 Hz, 1H), 7.02 (s, 1H), 6.97 (d, J=8 Hz, 1H), 4.23 (s, 2H), 3.49-3.45 (m, 2H), 3.23-3.17 (m, 2H), 2.71-2.61 (m, 2H), 2.35 (s, 3H), 1.77-1.75 (m, 2H), 1.46 (s, 6H). ESI-MS m/z: 322.0 [M+1]⁺.

### Step 4: Preparation of 1,1,5-trimethylspiro[isobenzofuran-3,4′-piperidine](C213)

To a stirred solution of 1′-benzyl-1,1,5-trimethyl-spiro[isobenzofuran-3,4′-piperidine](2.3 g, 7.15 mmol) in methanol (46 mL) was added Pd/C (1.5 g, 10% w/w, 1.4095 mmol) and AcOH (214 mg, 0.203 mL, 3.58 mmol). The reaction mixture was stirred under hydrogen atmosphere (30 psi) at ambient temperature for 24 hours. The reaction mixture was filtered over wetted celite pad, ensuring that the pad and palladium byproduct remained wet. Pad was washed with methanol (50 ml), and filtrate was concentrated. The crude residue was triturated with diethyl ether (3×50 ml), and concentrated to provide 1,1,5-trimethylspiro[isobenzofuran-3,4′-piperidine](1.429 g, 86%) ¹H NMR (400 MHz, DMSO-d6), δ ppm 8.27 (br, s, 1H), 7.15 (s, 2H), 6.92 (s, 1H), 3.32-3.29 (m, 2H), 3.15-3.08 (m, 2H), 2.34 (s, 3H), 2.17-2.06 (m, 2H), 1.79-1.67 (m, 2H), 1.44 (s, 6H). ESI-MS m/z: 232.2 [M+1]⁺.

### Step 5: Preparation of 1′-[(6-chloro-3-pyridyl)methyl]-1,1,5-trimethyl-spiro[isobenzofuran-3,4′-piperidine](C214)

A solution of 1,1,5-trimethylspiro[isobenzofuran-3,4′-piperidine](200 mg, 0.865 mmol) in DCE (5 mL) was treated with 2-chloro-5-(chloromethyl)pyridine (142 mg, 0.878 mmol) followed by DIPEA (407 μL, 2.337 mmol). The resulting solution was heated at 60° C. overnight. The reaction was cooled to ambient temperature then quenched by partitioning between 1 M NaOH and DCE. The combined organics were collected through a phase separator tube, then concentrated in vacuo. The crude residue was purified by flash column chromatography (Gradient: 0-100% EtOAc in Heptanes) to afford 1′-[(6-chloro-3-pyridyl)methyl]-1,1,5-trimethyl-spiro[isobenzofuran-3,4′-piperidine](130 mg, 42%) ¹H NMR (400 MHz, Chloroform-d) δ 8.35 (dd, J=2.4, 0.7 Hz, 1H), 7.69 (dd, J=8.2, 2.4 Hz, 1H), 7.30 (dd, J=8.2, 0.7 Hz, 1H), 7.10 (ddd, J=7.7, 1.6, 0.7 Hz, 1H), 6.98 (d, J=7.7 Hz, 1H), 6.91 (dt, J=1.6, 0.8 Hz, 1H), 3.56 (s, 2H), 2.78-2.72 (m, 2H), 2.52 (td, J=11.9, 11.5, 2.5 Hz, 2H), 2.37 (d, J=0.8 Hz, 3H), 2.01-1.93 (m, 2H), 1.70-1.64 (m, 2H), 1.48 (s, 6H). ESI-MS m/z: 357.32 [M+1]⁺.

### Step 6: Preparation of 2-methyl-1-[[5-[(1,1,5-trimethylspiro[isobenzofuran-3,4′-piperidine]-1′-yl)methyl]-2-pyridyl]oxy]propan-2-amine (288)

A 2 mL microwave vial was charged with 1′-[(6-chloro-3-pyridyl)methyl]-1,1,5-trimethyl-spiro[isobenzofuran-3,4′-piperidine](50 mg, 0.1398 mmol), 2-amino-2-methyl-propan-1-ol (9 μL, 0.1677 mmol), tBu-XPhos Pd G1 precatalyst (5 mg, 0.008 mmol), in tBuOH (1.5 mL). The mixture was degassed under nitrogen balloon, followed by addition of 2-methylpropan-2-olate (Sodium salt) (154 μL of 2 M, 0.3080 mmol). The vial was sealed and heated at 80° C. for 1 hour, then quenched by addition of methanol. The solution was concentrated and purified by reverse phase HPLC (Gradient: 10-90% MeCN in aqueous HCl (0.1% HCl)) to provide 2-methyl-1-[[5-[(1,1,5-trimethylspiro[isobenzofuran-3,4′-piperidine]-1′-yl)methyl]-2-pyridyl]oxy]propan-2-amine 288 (6.7 mg, 11%) ¹H NMR (300 MHz, Chloroform-d) δ 8.10 (dd, J=2.5, 0.7 Hz, 1H), 7.76 (dd, J=8.5, 2.4 Hz, 1H), 7.09 (ddd, J=7.7, 1.5, 0.8 Hz, 1H), 7.01 (d, J=7.7 Hz, 1H), 6.93-6.88 (m, 2H), 4.18 (s, 2H), 3.59 (s, 2H), 2.81 (d, J=11.0 Hz, 2H), 2.58-2.48 (m, 2H), 2.34 (s, 3H), 2.00 (td, J=13.1, 4.5 Hz, 2H), 1.68-1.60 (m, 2H), 1.39 (d, J=33.6 Hz, 12H). ESI-MS m/z: 410.47 [M+1]⁺.

### Compound 289

### 1,1,5-trimethyl-1′-[[1-(2-methylsulfonylethyl)pyrazol-4-yl]methyl]spiro[isobenzofuran-3,4′-piperidine](289)

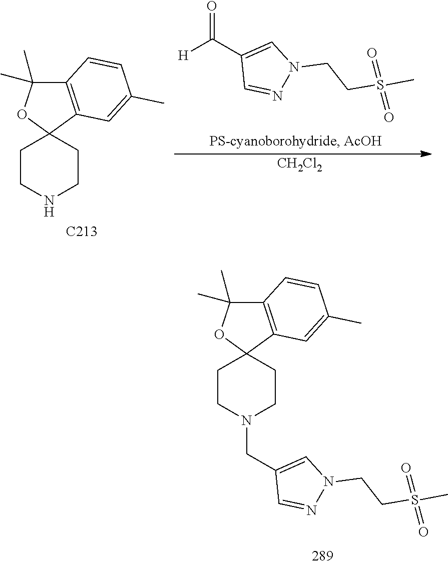

1,1,5-trimethylspiro[isobenzofuran-3,4′-piperidine]C213 (100 mg, 0.4323 mmol) and 1-(2-methylsulfonylethyl)pyrazole-4-carbaldehyde (131 mg, 0.6478 mmol) were dissolved in dichloromethane (2.25 mL). Acetic acid (130 mg, 2.165 mmol) was added, and the solution was transferred to a microwave tube (5 mL). PS-(trimethylammonio)methyl (cyanoborohydride) (650 mg of 2 mmol/g, 1.300 mmol) was added, the solution was capped, and heated to 110° C. in a microwave reactor for 1 hour. The solution was filtered and concentrated. The crude product was purified by reverse phase chromatography (Gradient: 20-80% MeCN in aqueous HCl (0.1% HCl) to provide 1,1,5-trimethyl-1′-[[1-(2-methylsulfonylethyl)pyrazol-4-yl]methyl]spiro[isobenzofuran-3,4′-piperidine](hydrochloride salt) 289 (120 mg, 61%) ESI-MS m/z: 418.18 [M+1]⁺.

### Compound 290

### (2′S)-2′,3,3,6-tetramethyl-1′-((1-(2-(methylsulfonyl)ethyl)-1H-1,2,3-triazol-4-yl)methyl)-3H-spiro[isobenzofuran-1,4′-piperidine](290)

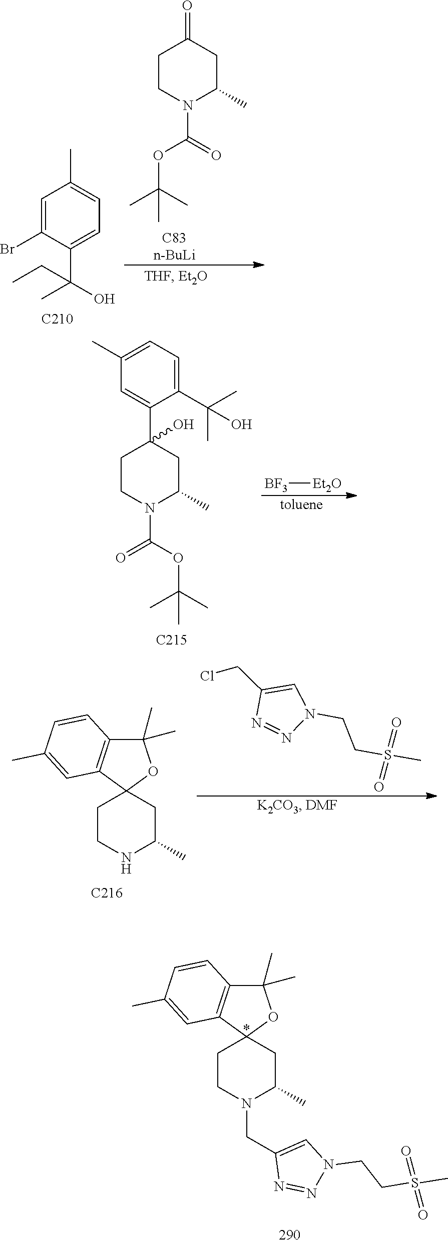

### Step 1: Preparation of tert-butyl (2S)-4-hydroxy-4-[2-(I-hydroxy-1-methyl-ethyl)-5-methyl-phenyl]-2-methyl-piperidine-1-carboxylate (C215)

To a stirred solution of 2-(2-bromo-4-methyl-phenyl)propan-2-ol (460 mg, 2.0 mmol) in THF (10 mL) was added n-BuLi (1.85 mL of 2.5 M, 4.62 mmol) slowly at −78° C. under nitrogen. The reaction mixture was stirred at the same temperature for 1 hour, then a solution of tert-butyl (S)-2-methyl-4-oxopiperidine-1-carboxylate (557 mg, 2.61 mmol) in THF (2 mL) was added. The reaction was stirred at ambient temperature for 16 hours. The reaction was quenched with saturated ammonium chloride, extracted with EtOAc (2×50 ml), dried over sodium sulfate, and concentrated to provide tert-butyl (2S)-4-hydroxy-4-[2-(1-hydroxy-1-methyl-ethyl)-5-methyl-phenyl]-2-methyl-piperidine-1-carboxylate (550 mg, 25%) ESI-MS m/z: 364.0 [M+1]⁺. The crude product was directly taken for next reaction without purification.

### Step 2: Preparation of (2′S)-1,1,2′,5-tetramethylspiro[isobenzofuran-3,4′-piperidine](C216)

To a stirred solution of tert-butyl (2S)-4-hydroxy-4-[2-(1-hydroxy-1-methyl-ethyl)-5-methyl-phenyl]-2-methyl-piperidine-1-carboxylate (3.2 g, 8.8 mmol) in Toluene (32 mL) was added BF₃·OEt₂ (6.24 g, 44.0 mmol) slowly at room temperature, then the reaction was stirred for 3 hours at ambient temperature. The mixture was then concentrated, washed with saturated NaHCO₃ (50 ml), extracted with EtOAc (2×50 ml), dried with sodium sulfate and concentrated, and crude was triturated with DEE (2×30 ml) to provide a crude solid that was purified by chiral SFC chromatography for isomer separation (Column: Chiralcel OX-H ((30×250 mm), 5p; Gradient: 70% CO₂ in methanol (15 mM Methanolic ammonia); Flow rate: 100 g/min; Temperature: 30.0° C.) to provide:

First eluting peak (2′S)-1,1,2′,5-tetramethylspiro[isobenzofuran-3,4′-piperidine]C216 (59 mg, 3%) ¹H NMR (400 MHz, DMSO-d6), δ ppm 7.08 (s, 2H), 6.91 (s, 1H), 2.91-2.79 (m, 3H), 2.31 (s, 3H), 1.78 (s, 1H), 1.70-1.62 (m, 1H), 1.49-1.33 (m, 9H), 0.95 (d, J=6.4 Hz, 3H). ESI-MS m/z: 246.0 [M+1]⁺.

Second eluting peak (2′S)-1,1,2′,5-tetramethylspiro[isobenzofuran-3,4′-piperidine]C217 (607 mg, 27%) ¹H NMR (400 MHz, DMSO-d6), δ ppm 7.33 (s, 1H), 7.09 (s, 2H), 3.07-2.88 (m, 3H), 2.32 (s, 3H), 1.86 (s, 1H), 1.72-1.65 (m, 3H), 1.40-1.38 (m, 7H), 0.99 (d, J=6.4 Hz, 3H). ESI-MS m/z: 246.0 [M+1]⁺.

### Step 3: Preparation of (2′S)-2′, 3, 3,6-tetramethyl-1′-((1-(2-(methylsulfonyl)ethyl)-1H-1,2, 3-triazol-4-yl)methyl)-3H-spiro[isobenzofuran-1,4′-piperidine](290)

To a stirred solution of (2′S)-1,1,2′,5-tetramethylspiro[isobenzofuran-3,4′-piperidine]C216 (27.43 mg, 0.11 mmol) and 4-(chloromethyl)-1-(2-methylsulfonylethyl)triazole (25 mg, 0.1118 mmol) in DMF was added K₂CO₃ (15.45 mg, 0.11 mmol). The solution was stirred 16 hours at 60° C. The reaction was extracted with DCM and water on a phase separator, then concentrated and purified by flash column chromatography (Gradient: 0-20% MeOH/DCM) to provide (2′S)-2′,3,3,6-tetramethyl-1′-((1-(2-(methylsulfonyl)ethyl)-1H-1,2,3-triazol-4-yl)methyl)-3H-spiro[isobenzofuran-1,4′-piperidine]290 (17.2 mg, 35%). ESI-MS m/z: 433.64 [M+1]⁺.

### Compound 291

### (2′S)-5-chloro-1,1,2′-trimethyl-1′-[[1-(2-methylsulfonylethyl)triazol-4-yl]methyl]spiro[isobenzofuran-3,4′-piperidine](291)

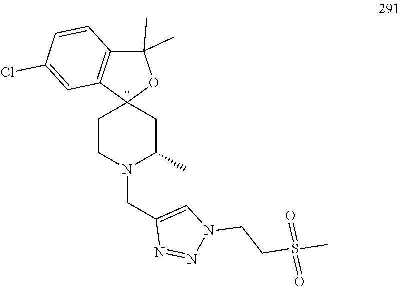 [SMILES: C[C@H]1CC2(CCN1Cc1cn(CCS(C)(=O)=O)nn1)OC(C)(C)c1ccc(Cl)cc12]

Compound 291 was prepared following the method described for compound 290 with the following modifications. In step 1, 2-(2-bromo-4-chloro-phenyl)propan-2-ol was used in the place of 2-(2-bromo-4-methyl-phenyl)propan-2-ol. In step 2, peak 2 was isolated and carried forward to the next step by chiral SFC chromatography for isomer separation (Column: Chiralcel OX-H ((30×250 mm), 5p; Gradient: 80% CO₂ in methanol (15 mM Methanolic ammonia); Flow rate: 90 g/min; Temperature: 30.0° C.). Step 3 provided (2′S)-5-chloro-1,1,2′-trimethyl-11′-[[1-(2-methylsulfonylethyl)triazol-4-yl]methyl]spiro[isobenzofuran-3,4′-piperidine]291 (4.3 mg, 19%). ESI-MS m/z: 453.18 [M+1]⁺.

### Compound 292

### (2′S)-5-chloro-2′-methyl-1′-[(1-phenylpyrazol-4-yl)methyl]spiro[1H-isobenzofuran-3 4′-piperidine]-1-carboxamide (292)

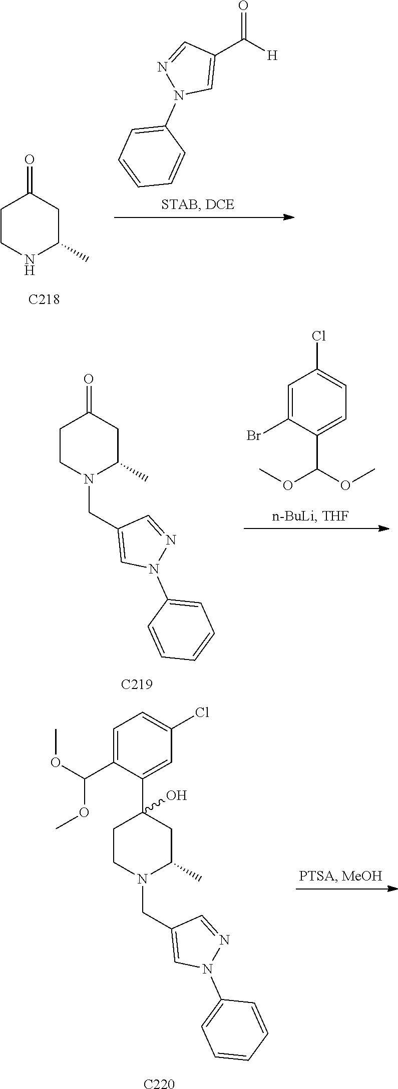

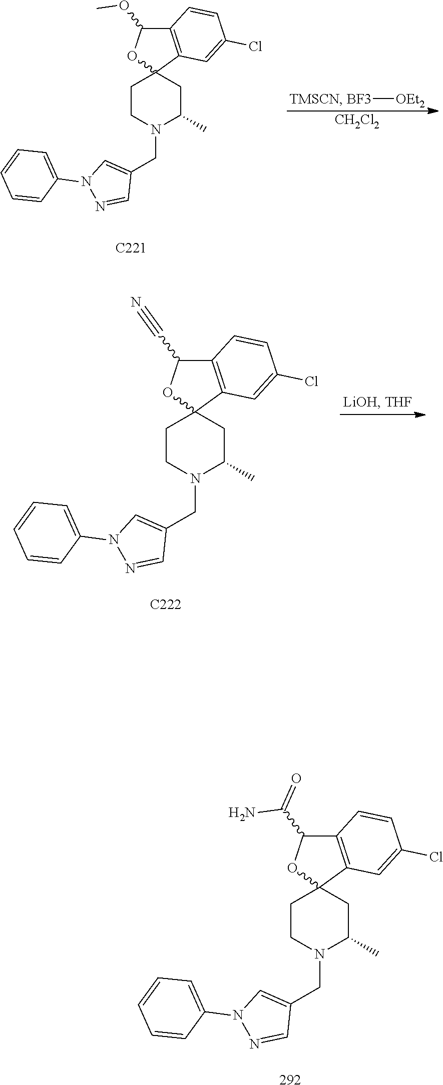

### Step 1: Preparation of (2S)-2-methyl-1-[(1-phenylpyrazol-4-yl)methyl]piperidin-4-one (C219)

To a stirred solution of 1-phenylpyrazole-4-carbaldehyde (2 g, 0.01 mol) in DCE (30 mL) was added (2S)-2-methylpiperidin-4-one (810 mg, 0.007 mol) followed by sodium triacetoxyborohydride (2.3 g, 0.01 mol) at 0° C. The reaction mixture was stirred for 4 hours at ambient temperature. The reaction was diluted with ethyl acetate (100 mL), washed with water (2×100 mL), and the organic layer dried over sodium sulfate and concentrated to provide crude compound (2S)-2-methyl-1-[(1-phenylpyrazol-4-yl)methyl]piperidin-4-one (2.2 g). This was directly carried forward to the next step without further purification. ESI-MS m/z: 270.06 [M+1]⁺.

### Step 2: Preparation of (2S)-4-[5-chloro-2-(dimethoxymethyl)phenyl]-2-methyl-1-[(1-phenylpyrazol-4-yl)methyl]piperidin-4-ol (C220)

To a stirred solution of 2-bromo-4-chloro-1-(dimethoxymethyl)benzene (1.5 g, 0.005 mol) and (2S)-2-methyl-1-[(1-phenylpyrazol-4-yl)methyl]piperidin-4-one (1.6 g, 0.006 mol) in THF (15 mL) was added n-Butyllithium (5.5 mL of 2.5 M, 0.014 mol) at −78° C., and maintained the same temperature for 3 hours. The reaction mixture was quenched with saturated ammonium chloride solution (50 mL) and extracted with EtOAc (2×100 mL). The combined organic layer was dried over Na₂SO₄,and concentrated to obtain (2S)-4-[5-chloro-2-(dimethoxymethyl)phenyl]-2-methyl-1-[(1-phenylpyrazol-4-yl)methyl]piperidin-4-ol (2.5 g, 59%) ESI-MS m/z: 456.36 [M+1]⁺.

### Step 3: Preparation of (2′S)-5-chloro-1-methoxy-2′-methyl-1′-[(1-phenylpyrazol-4-yl)methyl]spiro[1H-isobenzofuran-3,4′-piperidine](C221)

To a stirred solution of (2S)-4-[5-chloro-2-(dimethoxymethyl)phenyl]-2-methyl-1-[(1-phenylpyrazol-4-yl)methyl]piperidin-4-ol (2.5 g, 0.0030 mol) in MeOH (50 mL) was added PTSA (4.6 g, 0.0264 mol) at 0° C., and the reaction mixture was stirred at ambient temperature for 48 hours. The reaction mixture was concentrated, diluted with water (100 mL) and extracted with DCM (2×200 mL), organic layer was washed with 2 M NaOH solution (30 mL, pH=9) and dried over sodium sulfate, and concentrated. The crude residue was purified by flash column chromatography (Gradient: 60-100% EtOAc in hexanes) to provide (2′S)-5-chloro-1-methoxy-2′-methyl-1′-[(1-phenylpyrazol-4-yl)methyl]spiro[1H-isobenzofuran-3,4′-piperidine](1.1 g, 80%). ¹H NMR (400 MHz, DMSO-d6) δ 8.43 (d, J=8.0 Hz, 1H), 7.84 (d, J=8.4 Hz, 2H), 7.69 (d, J=11.2 Hz, 1H), 7.51-7.36 (m, 5H), 7.28 (t, J=8.0 Hz, 1H), 6.01-5.97 (m, 1H), 3.32-2.89 (m, 1H), 3.89-3.51 (m, 2H), 3.37-3.28 (m, 3H), 2.85-2.79 (m, 1H), 2.56-2.49 (m, 1H), 2.06-1.46 (m, 4H), 1.22-1.55 (m, 3H) ESI-MS m/z: 424.32 [M+1]⁺.

### Step 4: Preparation of (2′S)-5-chloro-2′-methyl-1′-[(1-phenylpyrazol-4-yl)methyl]spiro[1H-isobenzofuran-3,4′-piperidine]-1-carbonitrile (C222)

To a stirred solution of (2′S)-5-chloro-1-methoxy-2′-methyl-1′-[(1-phenylpyrazol-4-yl)methyl]spiro[1H-isobenzofuran-3,4′-piperidine](1.6 g, 0.0035 mol) in CH₂Cl₂ (50 mL) was added TMSCN (2.27 g, 3.2 mL, 0.0206 mol) followed by BF₃·OEt₂ (1.13 g, 1 mL, 0.008 mol) at −25° C. The solution was stirred at 0° C. for 1 hour. The reaction mixture was quenched with methanol (5 mL), followed by addition of 2 M NaOH sol. (pH=9). The organic layer was separated, dried over Na₂SO₄ and concentrated The crude residue was purified by flash column chromatography (Gradient: 30-40% EtOAc in hexanes), to afford (2′S)-5-chloro-2′-methyl-1′-[(1-phenylpyrazol-4-yl)methyl]spiro[1H-isobenzofuran-3,4′-piperidine]-1-carbonitrile (300 mg, 20%) ¹H NMR (300 MHz, DMSO-d6) δ 8.42 (s, 1H), 8.44 (s, 1H), 7.84 (d, J=8.4 Hz, 2H), 7.70 (s 1H), 7.58-7.46 (m, 5H), 7.28 (t, J=7.5 Hz, 1H), 6.23 (s, 1H), 3.66 (d, J=3.9 Hz, 1H), 3.05-3.01 (m, 1H), 2.85-2.80 (m, 1H), 2.65-2.69 (m, 1H), 2.12-1.92 (m, 2H), 1.77-1.65 (m, 2H), 1.23-1.15 (m, 3H). ESI-MS m/z 419.33 [M+1]⁺.

### Step 5: Preparation of (2′S)-5-chloro-2′-methyl-1′-[(1-phenylpyrazol-4-yl)methyl]spiro[1H-isobenzofuran-3,4′-piperidine]-1-carboxamide (292)

To a stirred solution of (2′S)-5-chloro-2′-methyl-1′-[(1-phenylpyrazol-4-yl)methyl]spiro[1H-isobenzofuran-3,4′-piperidine]-1-carbonitrile (100 mg, 226.77 μmol) in THF (2 mL) was added LiOH (60 mg, 0.0014 mol) in H₂O (0.5 mL) at ambient temperature. The solution was stirred for 16 hours. The reaction was concentrated, and the crude residue was dissolved in EtOAc (50 mL). The organic layer was dried over Na₂SO₄ and concentrated. The crude compound was purified by reverse phase HPLC (Gradient: 30-90% MeCN in 10 mM ammonium bicarbonate) to provide (2′S)-5-chloro-2′-methyl-1′-[(1-phenylpyrazol-4-yl)methyl]spiro[1H-isobenzofuran-3,4′-piperidine]-1-carboxamide 292 (20 mg, 19%), ¹H NMR (400 MHz, DMSO-d6): δ 8.45 (s, 1H), 7.84 (d, J=8.0 Hz, 2H), 7.71 (s, 1H), 7.48 (t, J=7.6 Hz, 2H), 7.41-7.26 (m, 5H), 7.19-6.98 (m, 1H), 5.34 (s, 1H), 3.72 (s, 2H), 2.98-2.81 (m, 2H), 2.67-2.51 (m, 1H), 1.94-1.66 (m, 4H), 1.30-1.10 (m, 3H). ESI-MS m/z: 437.39 [M+1]⁺.

### Compound 293

### (2′S)-5-chloro-2′-methyl-1′-[(1-phenylpyrazol-4-yl)methyl]spiro[1H-isobenzofuran-3,4′-piperidine]-1-carboxamide (293)

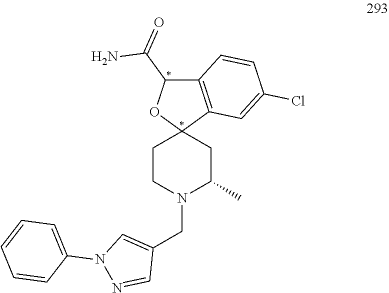 [SMILES: C[C@H]1CC2(CCN1Cc1cnn(-c3ccccc3)c1)OC(C(N)=O)c1ccc(Cl)cc12]

Compound 292 was purified by chiral SFC purification (Column: Chiralpak IC (250×30×5μ); Gradient: 50% CO₂ (0.5% IPAmine in IPA) in methanol; Flow rate: 70 g/min; Temperature: 30.0° C.) to provide (2′S)-5-chloro-2′-methyl-1′-[(1-phenylpyrazol-4-yl)methyl]spiro[1H-isobenzofuran-3,4′-piperidine]-1-carboxamide 293 (5 mg, 4%), ESI-MS m/z 437.42 [M+1]⁺.

### Compound 294

### (2′S)-5-chloro-N,2′-dimethyl-1′-[(1-phenylpyrazol-4-yl)methyl]spiro[1H-isobenzofuran-3,4′-piperidine]-1-carboxamide (294)

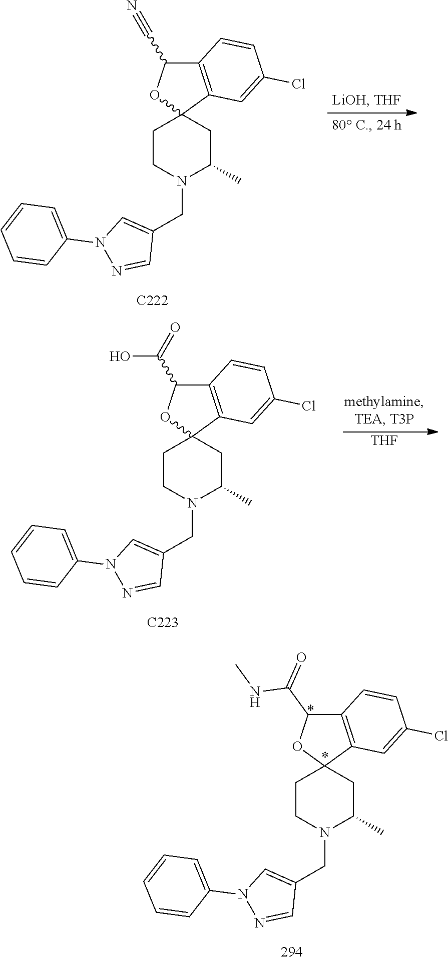

### Step 1: Preparation of (2′S)-5-chloro-2′-methyl-1′-[(1-phenylpyrazol-4-yl)methyl]spiro[1H-isobenzofuran-3,4′-piperidine]-1-carboxylic acid (C223)

To a stirred solution of (2′S)-5-chloro-2′-methyl-1′-[(1-phenylpyrazol-4-yl)methyl]spiro[1H-isobenzofuran-3,4′-piperidine]-1-carbonitrile (350 mg, 818.77 μmol) in THF (2 mL) was added LiOH (710 mg, 0.0166 mol) in H₂O (0.5 mL) at ambient temperature, and then stirred at 80° C. for 24 hours. The reaction was concentrated, and the crude residue was dissolved in water (50 mL) and extracted with EtOAc (30 mL). The separated organic layer and aqueous layer was acidified with 1 MHCl, and extracted with 10% MeOH in DCM (2×50 mL). The combined organic layer was dried over Na₂SO₄, and concentrated to provide (2′S)-5-chloro-2′-methyl-1′-[(1-phenylpyrazol-4-yl)methyl]spiro[1H-isobenzofuran-3,4′-piperidine]-1-carboxylic acid (300 mg, 67%) ESI-MS m/z 438.12 [M+1]⁺.

### Step 2: Preparation of (2′S)-5-chloro-N,2′-dimethyl-1′-[(1-phenylpyrazol-4-yl)methyl]spiro[1H-isobenzofuran-3,4′-piperidine]-1-carboxamide (294)

To a stirred solution of (2′S)-5-chloro-2′-methyl-1′-[(1-phenylpyrazol-4-yl)methyl]spiro[1H-isobenzofuran-3,4′-piperidine]-1-carboxylic acid (200 mg, 431.59 μmol) in THF (4 mL) was added methylamine (20 mg, 290.29 μmol) and TEA (294.00 mg, 0.4 mL, 0.0028 mol), followed by T3P (0.9 mL of 50% w/v, 0.0014 mol) at 0° C., and then the solution was allowed to warm to ambient temperature for 16 hours. The reaction mixture was dissolved in EtOAc (50 mL), then the organic layer was washed with 1 M NaOH solution (2×10 mL) and dried over Na₂SO₄ then concentrated. The crude residue was purified by chiral SFC purification (Column: Lux; Cellulose-4 (250×30×5μ); Gradient: 60% CO₂ (30 mM Methanolic ammonia in Ethanol) in methanol; Flow rate: 70 g/min; Temperature: 30.0° C.) and the first eluting peak was isolated to provide (2′S)-5-chloro-N,2′-dimethyl-1′-[(1-phenylpyrazol-4-yl)methyl]spiro[1H-isobenzofuran-3,4′-piperidine]-1-carboxamide 294 (10 mg, 5%), ¹H NMR (400 MHz, DMSO-d6): δ 8.45 (s, 1H), 7.84 (d, J=8.0 Hz, 2H), 7.71-7.68 (m, 2H), 7.49 (t, J=8.0 Hz, 2H), 7.40 (d, J=8.0 Hz, 1H), 7.34-7.27 (m, 3H), 5.39 (s, 1H), 3.69 (s, 2H), 2.93 (br s, 2H), 2.61 (d, J=4.4 Hz, 4H), 1.94-1.90 (m, 3H), 1.66 (br s 1H), 1.19 (s, 3H), ESI-MS m/z: 451.2 [M+1]⁺.

### Compound 295

### 5-chloro-N-methyl-1′-[[1-[3-(trifluoromethyl)phenyl]pyrazol-4-yl]methyl]spiro[1H-isobenzofuran-3,4′-piperidine]-1-carboxamide (295)

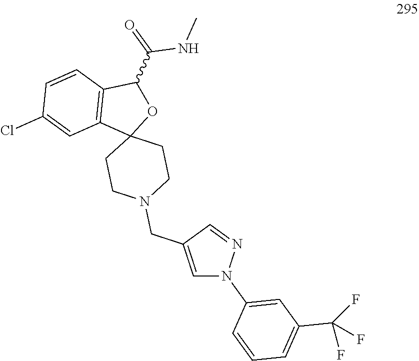 [SMILES: CNC(=O)C1OC2(CCN(Cc3cnn(-c4cccc(C(F)(F)F)c4)c3)CC2)c2cc(Cl)ccc21]

5-chloro-N-methyl-1′-[[1-[3-(trifluoromethyl)phenyl]pyrazol-4-yl]methyl]spiro[1H-isobenzofuran-3,4′-piperidine]-1-carboxamide (295) was prepared using the same HATU amide coupling method as used to prepare compound 294 with the appropriate carboxylic acid.

¹H NMR (400 MHz, DMSO-d6) δ 8.65 (s, 1H), 8.18 (d, J=9.3 Hz, 2H), 7.81-7.57 (m, 4H), 7.42-7.31 (m, 3H), 5.40 (s, 1H), 3.58 (s, 3H), 2.85 (s, 2H), 2.62 (d, J=4.7 Hz, 3H), 2.43-2.05 (m, 2H), 1.85 (d, J=13.6 Hz, 2H), 1.66 (d, J=13.7 Hz, 1H). LCMS m/z 505.27 [M+1]⁺.

### Compound 296

### 6-methoxy-1′-((1-phenyl-1H-pyrazol-4-yl)methyl)-3H-spiro[isobenzofuran-1,4′-piperidine]-3-carboxamide (296)

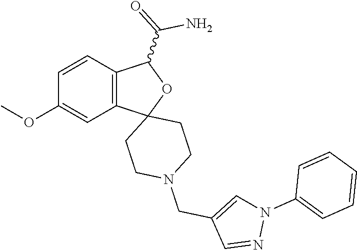 [SMILES: COc1ccc2c(c1)C1(CCN(Cc3cnn(-c4ccccc4)c3)CC1)OC2C(N)=O]

6-methoxy-1′-((1-phenyl-1H-pyrazol-4-yl)methyl)-3H-spiro[isobenzofuran-1,4′-piperidine]-3-carboxamide 296 was prepared in a similar manner for the preparation of compound 191 using 2-bromo-1-(dimethoxymethyl)-4-methoxybenzene to provide the final product in 14 mg.

¹H NMR (400 MHz, DMSO-d6): δ 8.41 (s, 1H), 7.83 (d, J=8.8 Hz, 2H), 7.67 (s, 1H), 7.48 (t, J=7.6 Hz, 2H), 7.32-7.24 (m, 3H), 7.0 (brs, 1H), 6.85-6.83 (m, 2H), 5.26 (s, 1H), 3.74 (s, 3H), 3.51 (s, 2H), 2.80 (brs, 2H), 2.49-2.32 (m, 2H), 2.10-2.02 (m, 1H), 1.92-1.79 (m, 2H), 1.65-1.62 (m, 1H). LCMS m/z 419.2 [M+1]⁺.

### Compound 297

### 6-methoxy-N-methyl-1′-((1-phenyl-1H-pyrazol-4-yl)methyl)-3H-spiro[isobenzofuran-1,4′-piperidine]-3-carboxamide (297)

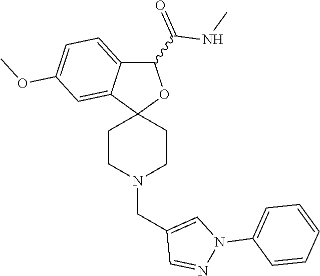 [SMILES: CNC(=O)C1OC2(CCN(Cc3cnn(-c4ccccc4)c3)CC2)c2cc(OC)ccc21]

6-methoxy-N-methyl-1′-((1-phenyl-1H-pyrazol-4-yl)methyl)-3H-spiro[isobenzofuran-1,4′-piperidine]-3-carboxamide was prepared in a similar manner to compound 195 using 2-bromo-1-(dimethoxymethyl)-4-methoxybenzene to provide the final product 297 in 30 mg.

¹H NMR (400 MHz, DMSO-d6) δ 8.41 (s, 1H), 7.83 (d, J=8.0, 2H), 7.67 (s, 1H), 7.48 (t, J=8.0 Hz, 3H), 7.30-7.22 (m, 2H), 6.84-6.82 (m, 2H), 5.31 (s, 1H), 3.74 (s, 3H), 3.52 (s, 2H), 2.80 (t, J=12.0 Hz, 2H), 2.61 (d, J=8.0 Hz, 3H), 2.52-2.47 (m, 1H), 2.33 (t, J=11.2 Hz, 1H), 2.11-2.04 (m, 2H), 1.89 (s, 1H). LCMS m/z 433.51 [M+1]⁺.

### Compound 298

### 6-chloro-1′-((1-isopropyl-1H-pyrazol-4-yl)methyl)-N-methyl-3H-spiro[isobenzofuran-1,4′-piperidine]-3-carboxamide (298)

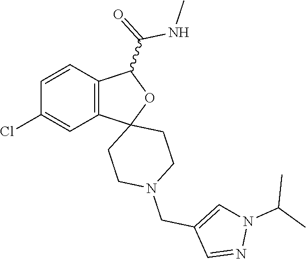 [SMILES: CNC(=O)C1OC2(CCN(Cc3cnn(C(C)C)c3)CC2)c2cc(Cl)ccc21]

Compound 298 was prepared in a similar manner to compound 195 using 1-isopropyl-1H-pyrazole-4-carbaldehyde to provide the final product 6-chloro-1′-((1-isopropyl-1H-pyrazol-4-yl)methyl)-N-methyl-3H-spiro[isobenzofuran-1,4′-piperidine]-3-carboxamide (12 mg). ¹H NMR (400 MHz, DMSO-d6): δ 7.63 (s, 1H), 7.60-7.48 (m, 1H), 7.40 (d, J=1.6 Hz, 1H), 7.38-7.32 (m, 3H), 5.39 (s, 1H), 4.47-4.41 (m, 1H), 3.39 (s, 2H), 2.74 (brs, 2H), 2.62 (d, J=4.8 Hz, 3H), 2.51-2.49 (m, 1H), 2.46-2.40 (m, 1H), 2.07-2.01 (m, 1H), 1.84-1.79 (m, 2H), 1.62-1.59 (m, 1H), 1.39 (d, J=6.8 Hz, 6H). LCMS m/z 403.42 [M+1]⁺.

### Compound 299

### 6-chloro-N-methyl-1′-((5-methyl-1-phenyl-1H-pyrazol-4-yl)methyl)-3H-spiro[isobenzofuran-1,4′-piperidine]-3-carboxamide (299)

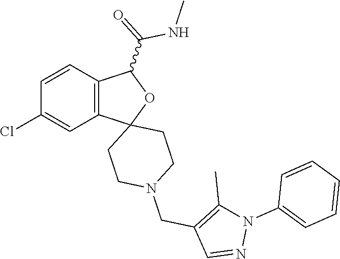 [SMILES: CNC(=O)C1OC2(CCN(Cc3cnn(-c4ccccc4)c3C)CC2)c2cc(Cl)ccc21]

Compound 299 was prepared in a similar manner to compound 190 using 2-bromo-4-chloro-1-(dimethoxymethyl)benzene and 5-methyl-1-phenyl-1H-pyrazole-4-carbaldehyde to provide the final product 6-chloro-N-methyl-1′-((5-methyl-1-phenyl-1H-pyrazol-4-yl)methyl)-3H-spiro[isobenzofuran-1,4′-piperidine]-3-carboxamide (2 mg). LCMS m/z 451.27 [M+1]⁺.

### Compound 300 and 301

### 5-chloro-1′-[(1-methylpyrazol-4-yl)methyl]spiro[indane-3,4′-piperidine]-1-ol (300) and 6-chloro-1′-[(1-methylpyrazol-4-yl)methyl]spiro[indane-1,4′-piperidine]-2-ol (301)

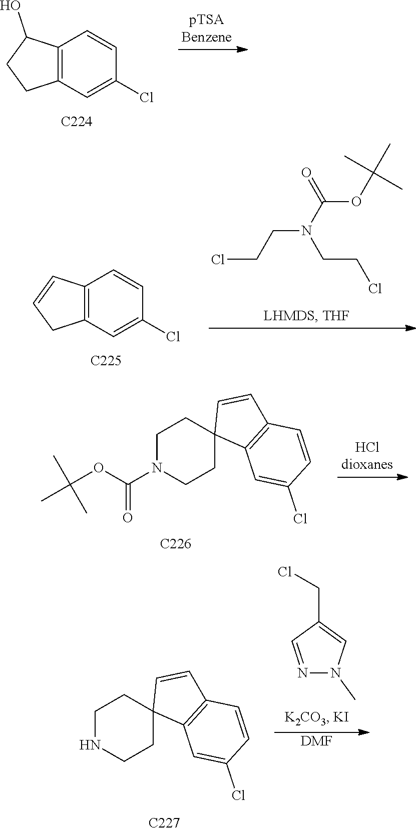

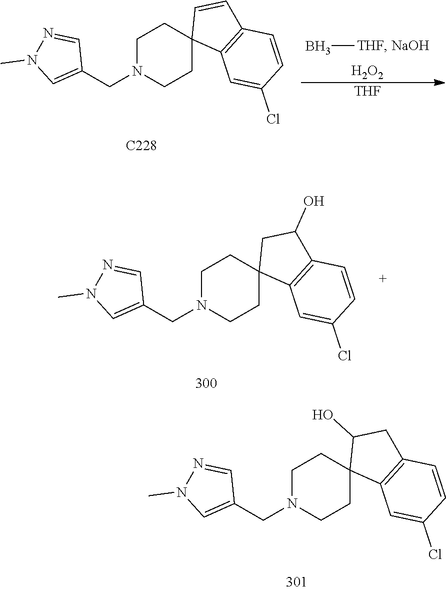

### Step 1: Preparation of 6-chloro-1H-indene (C225)

To a stirred solution 5-chloroindan-1-ol (10.0 g, 0.0593 mol) in benzene (100 mL) was added pTSA (206 mg, 0.193 mL, 0.0012 mol) at ambient temperature, then the reaction was stirred at 80° C. for 1 hour. The reaction mixture was cooled to ambient temperature, basified with saturated sodium bicarbonate solution (100 ml), added water (50 ml), extracted with ethyl acetate (2×200 ml), combined organic layers, and dried with sodium sulfate. The organic layer was filtered and concentrate. Crude residue was purified by column (Gradient: 100% hexanes) to afford 6-chloro-1H-indene (6.2 g, 68%). ¹H NMR (400 MHz, CDCl₃), δ ppm 7.43 (s, 1H), 7.29-7.27 (m, 1H), 7.24-7.22 (m, 1H), 6.84-6.81 (m, 1H), 6.55-6.53 (m, 1H), 3.37 (s, 2H).

### Step 2: Preparation of tert-butyl 6-chlorospiro[indene-1,4′-piperidine]-1′-carboxylate (C226)

To a stirred solution of 6-chloro-1H-indene (4.0 g, 0.01 mol) in THF (40 mL) was added LHMDS (23.2 mL of 1 M, 0.0232 mol) at 0° C. over 20 minutes. Reaction was stirred at 0° C. for 1 hour, then tert-butyl N,N-bis(2-chloroethyl)carbamate (2.25 g, 0.01 mol) in THF (10 ml) was added slowly at 0° C. over 10 minutes, then stirred at 0° C. for 2 hours. The reaction was warmed to ambient temperature, and stirred for 16 hours. The reaction mixture was concentrated, and, purified by column chromatography (Gradient: 5% ethyl acetate in hexanes) to afford tert-butyl 6-chlorospiro[indene-1,4′-piperidine]-1′-carboxylate (5.0 g, 44%). ¹H NMR (400 MHz, CDCl₃), δ ppm 7.30-7.26 (m, 1H), 7.23-7.15 (m, 2H), 6.90-6.83 (m, 1H), 6.74-6.72 (m, 1H), 4.18-4.13 (m, 2H), 3.13-3.07 (m, 2H), 2.10-1.93 (m, 2H), 1.15-1.45 (m, 9H), 1.34-1.31 (m, 2H). LCMS m/z 264.0 [M−55]⁺.

### Step 3: Preparation of 6-chlorospiro[indene-1,4′-piperidine](C227)

To a stirred solution of tert-butyl 6-chlorospiro[indene-1,4′-piperidine]-1′-carboxylate (300 mg, 919.25 μmol) in 1,4-Dioxane (3.0 mL) was added HCl (4 M in 1,4-Dioxane) (2.30 mL of 4 M, 0.0092 mol) at ambient temperature, then the reaction was stirred for 3 hours. The reaction mixture was concentrated to provide 6-chlorospiro[indene-1,4′-piperidine](Hydrochloric acid salt) (230 mg, 95%). ¹H NMR (300 MHz, DMSO d6), δ ppm 9.12 (br, s, 1H), 7.41-7.32 (m, 3H), 7.18 (d, J=5.7 Hz, 1H), 6.86 (d, J=5.4 Hz, 1H), 3.42-3.38 (m, 2H), 3.27-3.19 (m, 2H), 2.34 (t, J=11.1 Hz, 2H), 1.34 (d, J=14.1 Hz, 2H). LCMS m/z: 220.0 [M+1]⁺.

### Step 4: Preparation of 6-chloro-1′-[(1-methylpyrazol-4-yl)methyl]spiro[indene-1,4′-piperidine](C228)

To a stirred solution of 6-chlorospiro[indene-1,4′-piperidine](Hydrochloric acid salt) (230 mg, 869.92 μmol) in DMF (4.6 mL) was added 4-(chloromethyl)-1-methyl-pyrazole (Hydrochloric acid salt) (145.31 mg, 869.92 μmol), KI (144.41 mg, 869.92 μmol) and K₂CO₃ (594.29 mg, 0.0043 mol) at ambient temperature. Then the reaction was stirred at 80° C. for 16 hours. The reaction was quenched with water (50 ml), extracted with ethyl acetate (2×50 ml), and organic layers were dried with sodium sulfate, filtered, and concentrated to provide 6-chloro-1′-[(1-methylpyrazol-4-yl)methyl]spiro[indene-1,4′-piperidine](240 mg, 58%). LCMS m/z: 314.0 [M+1]⁺.

### Step 5: Preparation of 5-chloro-1′-[(1-methylpyrazol-4-yl)methyl]spiro[indane-3,4′-piperidine]-1-ol (300) and 6-chloro-1′-[(1-methylpyrazol-4-yl)methyl]spiro[indane-1,4′-piperidine]-2-ol (301)

To a stirred solution of 6-chloro-1′-[(1-methylpyrazol-4-yl)methyl]spiro[indene-1,4′-piperidine](240 mg, 749.46 μmol) in THF (3.6 mL) was added BH₃ (solution in THF) (1.9 mL of 1 M, 0.0019 mol) at −5° C. The reaction mixture was stirred at ambient temperature for 1 hour. NaOH (5.0 mL of 3 M, 0.0150 mol) and H₂O₂ (2.55 mL of 30% w/v, 0.0225 mol) were added at 0° C. over 10 minutes. The reaction was stirred at ambient temperature for 2 hours. The reaction mixture was quenched with water (30 ml), extracted with ethyl acetate (2×30 ml), and combined organic layers were washed with water (50 ml), dried with sodium sulfate, and concentrated. The regioisomers were separated and purified by prep HPLC (Gradient: 0-20% MeCN in 10 mM ammonium bicarbonate) to provide:

The first eluting peak as 5-chloro-1′-[(1-methylpyrazol-4-yl)methyl]spiro[indane-3,4′-piperidine]-1-ol (300) (8 mg, 3%). ¹H NMR (400 MHz, DMSO d6), δ ppm 7.56 (s, 1H), 7.31-7.29 (m, 2H), 7.25-7.23 (m, 2H), 5.30 (br s, 1H), 5.02 (t, J=6.4 Hz, 1H), 3.79 (s, 3H), 3.34 (s, 2H), 2.74-2.76 (m, 2H), 2.49-2.35 (m, 1H), 2.09-1.92 (m, 3H), 1.68-1.61 (m, 2H), 1.50 (d, J=11.6 Hz, 1H), 1.32 (d, J=11.2 Hz, 1H). LCMS m/z: 332.0 [M+1]⁺.

The second eluting peak as 6-chloro-1′-[(1-methylpyrazol-4-yl)methyl]spiro[indane-1,4′-piperidine]-2-ol (301) (16 mg, 6%). ¹H NMR (400 MHz, DMSO d6), δ ppm 7.55 (s, 1H), 7.29 (s, 1H), 7.20-7.13 (m, 3H), 4.79 (br s, 1H), 4.27 (d, J=4 Hz, 1H), 3.79 (s, 3H), 3.32 (s, 2H), 3.16-3.09 (m, 1H), 2.68-2.62 (m, 3H), 2.24-2.19 (m, 2H), 1.95 (d, J=13.2 Hz, 1H), 1.78-1.71 (m, 1H), 1.43-1.41 (m, 2H). LCMS m/z: 332.0 [M+1]⁺. Both regioisomers was confirmed by nuclear Overhauser effect.

### Compound 302

### 5-chloro-1′-[[1-(2-methylsulfonylethyl)pyrazol-4-yl]methyl]spiro[indane-3,4′-piperidine]-1-ol (302)

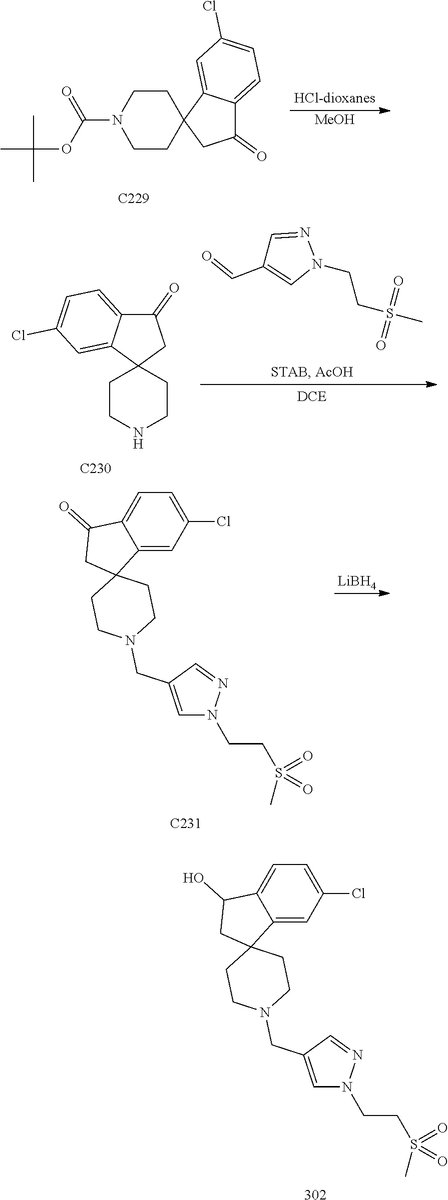

### Step 1: Preparation of 5-chlorospiro[indane-3,4′-piperidine]-1-one (C230)

A solution of tert-butyl 6-chloro-3-oxo-spiro[indane-1,4′-piperidine]-1′-carboxylate (540 mg, 1.61 mmol) in 4 mL methanol and HCl (2 mL of 4 M, 8.000 mmol)-dioxane solution was stirred at 50° C. for 1 hour. The reaction mixture was concentrated to provide the product 5-chlorospiro[indane-3,4′-piperidine]-1-one (Hydrochloride salt) (435 mg, 98%). LCMS m/z: 236.11 [M+1]⁺.

### Step 2: Preparation of 5-chloro-1′-[[1-(2-methylsulfonylethyl)pyrazol-4-yl]methyl]spiro[indane-3,4′-piperidine]-1-one (C231)

To 5-chlorospiro[indane-3,4′-piperidine]-1-one (HCl salt) (165 mg, 0.6002 mmol), 1-(2-methylsulfonylethyl)pyrazole-4-carbaldehyde (145 mg, 0.6173 mmol), and AcOH (120 μL, 2.110 mmol)) in 4 mL DCE, was added STAB (386 mg, 1.830 mmol) and stirred at ambient temperature for 6 hours. Solution was neutralized and concentrated, then purified by flash column chromatography (Gradient: 0-20% MeOH/DCM) to provide 5-chloro-1′-[[1-(2-methylsulfonylethyl)pyrazol-4-yl]methyl]spiro[indane-3,4′-piperidine]-1-one (110 mg, 40%) LCMS m/z: 422.22 [M+1]⁺.

### Step 3: Preparation of 5-chloro-1′-[[1-(2-methylsulfonylethyl)pyrazol-4-yl]methyl]spiro[indane-3,4′-piperidine]-1-ol (302)

To 5-chloro-1′-[[1-(2-methylsulfonylethyl)pyrazol-4-yl]methyl]spiro[indane-3,4′-piperidine]-1-one (24 mg, 0.05462 mmol) in methanol (2 mL) was added LiBH₄ (15 mg, 0.6886 mmol). The reaction was stirred at ambient temperature for 12 hours, then concentrated. Purification by reverse phase HPLC (0-50% MeCN in 0.1% formic acid) provided 5-chloro-1′-[[1-(2-methylsulfonylethyl)pyrazol-4-yl]methyl]spiro[indane-3,4′-piperidine]-1-ol 302 (20.4 mg, 87%). ¹H NMR (300 MHz, Chloroform-d) δ 8.45 (s, 1H), 7.95 (s, 1H), 7.70 (s, 1H), 7.37 (d, J=8.0 Hz, 1H), 7.33-7.18 (m, 2H), 5.19 (dd, J=7.1, 5.0 Hz, 1H), 4.69 (dd, J=6.9, 5.6 Hz, 2H), 4.24 (s, 2H), 3.71 (t, J=6.2 Hz, 2H), 3.48 (d, J=12.3 Hz, 2H), 3.08 (ddt, J=12.9, 9.3, 5.2 Hz, 1H), 2.88 (s, 3H), 2.54 (dd, J=13.6, 7.2 Hz, 1H), 2.22 (td, J=13.8, 4.0 Hz, 1H), 2.12-1.83 (m, 3H), 1.78-1.55 (m, 1H). LCMS m/z: 423.98 [M+1]⁺.

### Compound 303

### 5-chloro-1-methyl-1′-[[i-(2-methylsulfonylethyl)pyrazol-4-yl]methyl]spiro[indane-3,4′-piperidine]-1-ol (303)

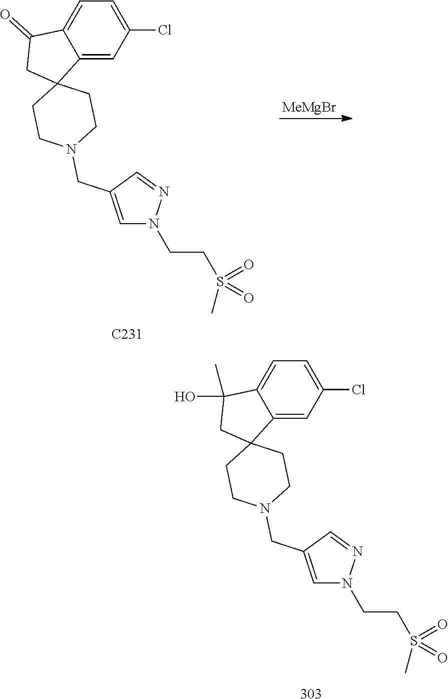 [SMILES: "MeMgBr" ; CS(=O)(=O)CCn1cc(CN2CCC3(CC2)CC(=O)c2ccc(Cl)cc23)cn1 ; "C231" ; CC1(O)CC2(CCN(Cc3cnn(CCS(C)(=O)=O)c3)CC2)c2cc(Cl)ccc21 ; "303"]

To 5-chloro-1′-[[1-(2-methylsulfonylethyl)pyrazol-4-yl]methyl]spiro[indane-3,4′-piperidine]-1-one (9.4 mg, 0.02139 mmol) in 2 mL diethyl ether was added bromo(methyl)magnesium (280 μL of 1.4 M, 0.3920 mmol). Reaction mixture was stirred at ambient temperature for 16 hours. Added 2 drops of saturated ammonium chloride and DCM (10 ml), then dried over Na₂SO₄. Organic layer was filtered, concentrated, and crude residue purified by reverse phase HPLC (Gradient: 0-50% MeCN in aqueous 0.1% TFA) to provide 5-chloro-1-methyl-1′-[[1-(2-methylsulfonylethyl)pyrazol-4-yl]methyl]spiro[indane-3,4′-piperidine]-1-ol (Trifluoroacetate salt) 303 (0.6 mg, 5%) ESI-MS m/z: 438.09 [M+1]⁺.

### Compound 304

### 5-methyl-1′-[(1-methylpyrazol-4-yl)methyl]spiro[1H-2-benzothiophene-3,4′-piperidine](304)

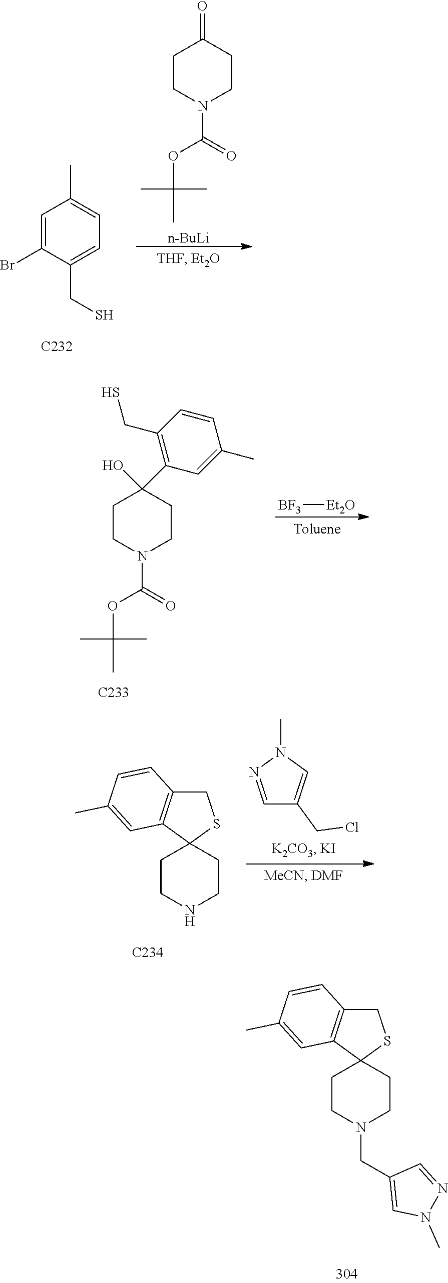

### Step 1: Synthesis of tert-butyl 4-hydroxy-4-[5-methyl-2-(sulfanylmethyl)phenyl]piperidine-1-carboxylate (C233)

To a stirred solution of (2-bromo-4-methyl-phenyl)methanethiol (C232) (2.0 g, 0.0083 mol) in THF (20 mL), n-BuLi in Hexane (8.32 mL of 2.5 M, 0.0208 mol) was added at −78° C. The reaction mixture was stirred for 1 hour. To the reaction mixture the solution of tert-butyl 4-oxopiperidine-1-carboxylate (1.6538 g, 0.0083 mol) in diethyl ether (6 mL) was added and the reaction mixture was stirred for 2 hours at −78° C. The reaction was quenched with NH₄Cl solution, and extracted with EtOAc (2×80 mL), the organic layer was washed with brine (50 mL) and dried over Na₂SO₄, concentrated. The crude residue was purified by reverse phase HPLC (Gradient: 0-20% MeCN in 10 mM ammonium bicarbonate) to provide tert-butyl 4-hydroxy-4-[5-methyl-2-(sulfanylmethyl)phenyl]piperidine-1-carboxylate (0.8 g, 28%) ¹H NMR (400 MHz, DMSO-d6) δ 7.25 (d, J=7.6 Hz, 1H), 7.12 (s, 1H), 7.03 (d, J=8 Hz, 1H), 5.12 (s, 1H), 4.09-4.06 (m, 2H), 3.84-3.82 (m, 2H), 3.167 (d, J=5.2 Hz, 2H), 2.67 (t, J=7.6 Hz, 1H), 2.26 (s, 3H), 1.86-1.80 (m, 4H), 1.41 (s, 9H). LCMS m/z: 338.2 (M+1)⁺.

### Step 2: Preparation of 5-methylspiro[1H-2-benzothiophene-3,4′-piperidine](C234)

To a stirred solution of tert-butyl 4-hydroxy-4-[5-methyl-2-(sulfanylmethyl)phenyl]piperidine-1 carboxylate (1.2 g, 0.0034 mol) in Toluene (15 mL) was added BF₃·OEt₂ (2.0580 g, 1.8212 mL, 0.0145 mol) at ambient temperature. The reaction mixture was stirred for 4 hours. The reaction mixture was concentrated. The crude residue was titurated with diethyl ether (2×10 mL) to afford 5-methylspiro[1H-2-benzothiophene-3,4′-piperidine](875 mg, 100%). ¹H NMR (400 MHz, DMSO-d6) δ 8.52 (br s, 1H), 8.35 (br s, 1H), 7.21 (d, J=8 Hz, 1H), 7.12 (d, J=7.6 Hz, 1H), 6.98 (s, 1H), 4.16 (s, 2H), 3.45 (d, J=13.2 Hz, 2H), 3.0 (q, J=12 Hz, 2H), 2.35-2.28 (m, 5H), 1.93 (d, J=13.6 Hz, 2H). LCMS m/z 220.36 [M+1]⁺.

### Step 3: Preparation of 5-methyl-1′-[(1-methylpyrazol-4-yl)methyl]spiro[1H-2-benzothiophene-3,4′-piperidine](304)

To a stirred solution of 5-methylspiro[1H-2-benzothiophene-3,4′-piperidine](300 mg, 0.0012 mol) in MeCN (6 mL) and DMF (1 mL) was added 4-(chloromethyl)-1-methyl-pyrazole (160 mg, 0.0011 mol) followed by the addition of K₂CO₃ (830 mg, 0.0060 mol) and catalytic amount of KI (40 mg, 240.96 μmol) at room temperature. The reaction mixture was stirred for 16 h at room temperature. The solvent was evaporated, and the crude compound was purified by reverse phase HPLC (Gradient: 0-20% MeCN in 0.1% TFA) to provide 5-methyl-1′-[(1-methylpyrazol-4-yl)methyl]spiro[1H-2-benzothiophene-3,4′-piperidine]304 (32 mg, 8%). ¹H NMR (400 MHz, DMSO-d6) δ 7.60 (br s, 1H), 7.34 (br s, 1H), 7.14 (d, J=7.6 Hz, 1H), 7.07-7.04 (m, 2H), 4.07 (s, 2H), 3.80 (s, 3H), 3.31 (s, 2H), 2.91 (br s, 2H), 2.32-2.14 (m, 7H), 1.75 (br s, 2H). LCMS m/z: 314.2 [M+1]⁺.

### Compound 305

### 5-chloro-1′-[[1-(2-methylsulfonylethyl)pyrazol-4-yl]methyl]spiro[indoline-3,4′-piperidine](305)

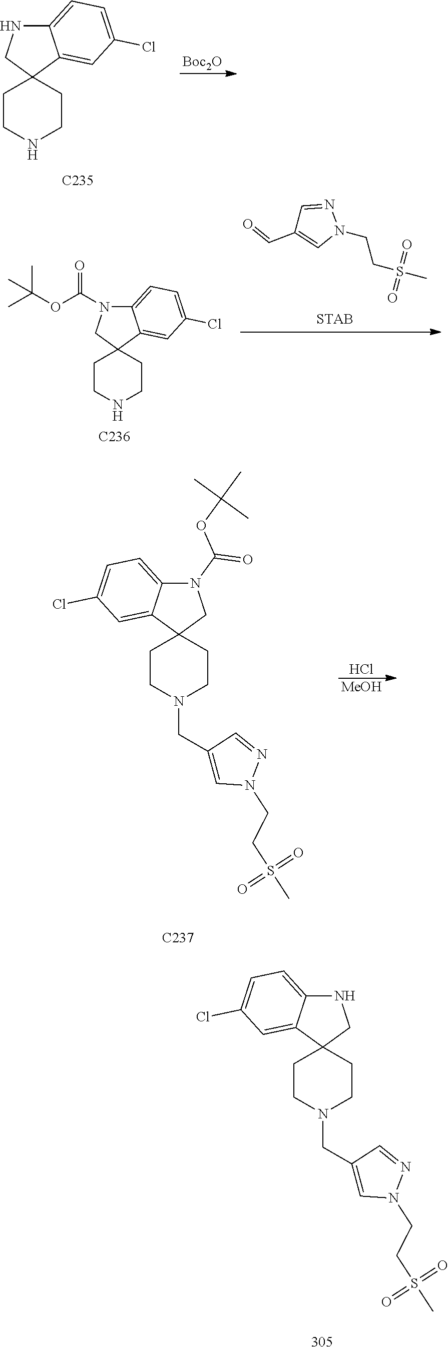

### Step 1: Preparation of tert-butyl 5-chlorospiro[indoline-3,4′-piperidine]-1-carboxylate (C236)

To a solution of 5-chlorospiro[indoline-3,4′-piperidine]C235 (170 mg, 0.7633 mmol) in AcOH (6.5 mL of 10% w/w) (aqueous) was added Boc₂O (174.9 mg, 184.1 μL, 0.8015 mmol) in 1,4-dioxane (6.5 mL) and the mixture was vigorously stirred 16 hours. It was diluted with water (10 mL), extracted with ethyl acetate (2×10 mL), dried over MgSO₄ and evaporated to give tert-butyl 5-chlorospiro[indoline-3,4′-piperidine]-1-carboxylate (160 mg, 65%) as a yellow oil which was used directly without further purification.

### Step 2: Preparation of tert-butyl 5-chloro-1′-[[1-(2-methylsulfonylethyl)pyrazol-4-yl]methyl]spiro[indoline-3,4′-piperidine]-1-carboxylate (C237)

To tert-butyl 5-chlorospiro[indoline-3,4′-piperidine]-1-carboxylate (Hydrochloride salt) (182 mg, 0.5066 mmol) in 3 mL DCE was added 1-(2-methylsulfonylethyl)pyrazole-4-carbaldehyde (126 mg, 0.5364 mmol) followed by STAB (214 mg, 1.015 mmol). The resulting mixture was stirred for 16 hours. The reaction was neutralized, concentrated, and purified by flash column chromatography (Gradient: 0 to 20% MeOH/DCM) to provide tert-butyl 5-chloro-1′-[[1-(2-methylsulfonylethyl)pyrazol-4-yl]methyl]spiro[indoline-3,4′-piperidine]-1-carboxylate (Acetic Acid Salt) (60 mg, 20%) ¹H NMR (300 MHz, Chloroform-d) δ 9.74 (s, 3H), 7.57 (d, J=13.3 Hz, 2H), 7.19-7.02 (m, 2H), 4.64 (dd, J=6.8, 5.5 Hz, 2H), 3.79 (s, 2H), 3.67 (dd, J=13.9, 7.7 Hz, 4H), 3.14 (d, J=11.8 Hz, 2H), 2.60 (s, 3H), 2.30 (t, J=12.2 Hz, 2H), 2.10 (s, 5H), 2.08 (s, 2H), 1.85-1.63 (m, 2H), 1.58 (s, 9H). LCMS m/z: 509.21 [M+1]⁺.

### Step 3: Preparation of 5-chloro-1′-[[1-(2-methylsulfonylethyl)pyrazol-4-yl]methyl]spiro[indoline-3,4′-piperidine](305)

A solution of tert-butyl 5-chloro-1′-[[1-(2-methylsulfonylethyl)pyrazol-4 yl]methyl]spiro[indoline-3,4′-piperidine]-1-carboxylate (Acetic Acid Salt) (4 mg, 0.006788 mmol) in 1 mL methanol and 0.5 ml 4 M HCl was stirred at 50° C. for 30 minutes. The solution was concentrated to provide 5-chloro-1′-[[1-(2-methylsulfonylethyl)pyrazol-4 yl]methyl]spiro[indoline-3,4′-piperidine](Dihydrochloride salt) 305 (3.2 mg, 93%). LCMS m/z: 409.12 [M+1]⁺.

### Compound 306 and 307

### (2′S)-5-chloro-2′-methyl-1′-[[1-(2-methylsulfonylethyl)pyrazol-4-yl]methyl]spiro[indane-3,4′-piperidine]-1-ol (306) and (2′S)-5-chloro-2′-methyl-1′-[[1-(2-methylsulfonylethyl)pyrazol-4-yl]methyl]spiro[indane-3,4′-piperidine]-1-ol (307)

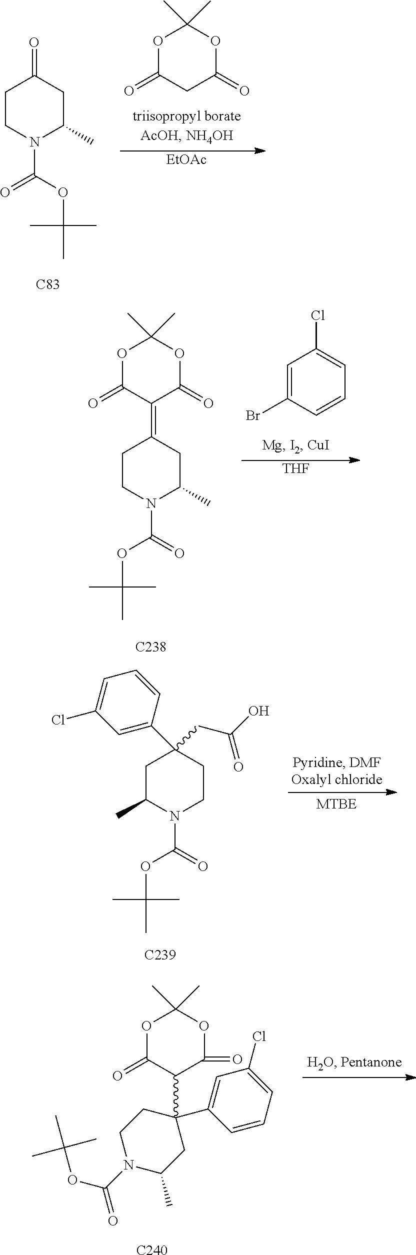

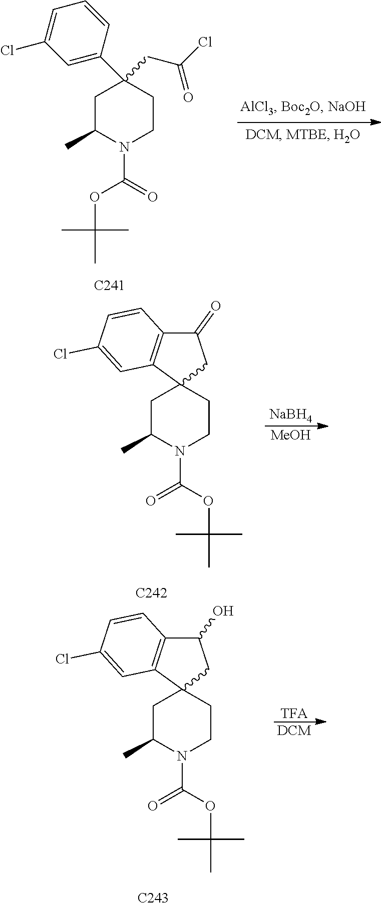

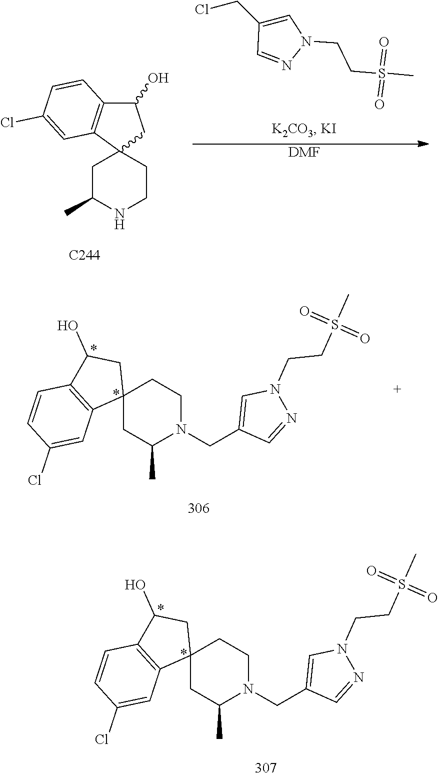

### Step 1: Preparation of tert-butyl (2S)-4-(2, 2-dimethyl-4,6-dioxo-1, 3-dioxan-5-ylidene)-2-methyl-piperidine-1-carboxylate (C238)

To a stirred solution of tert-butyl (2S)-2-methyl-4-oxo-piperidine-1-carboxylate (25 g, 0.1172 mol) in ethyl acetate (550 mL) was added 2,2-dimethyl-1,3-dioxane-4,6-dione (17 g, 0.1180 mol) at room temperature. After 5 minutes, triisopropyl borate (37.490 g, 46 mL, 0.1993 mol), NH₄OH (10.8 g, 12 mL of 25% w/v, 0.3082 mol) and acetic acid (6.33 g, 6 mL, 0.1055 mol) were slowly added. Reaction mixture was stirred at room temperature for 20 hours. Reaction mixture was cooled 0° C., stirred for 20 minutes and filtered, filtrate was concentrated under reduced pressure to obtained crude residue. Crude residue purified by flash column chromatography (Gradient: 15% EtOAc in Pet Ether) to afford tert-butyl (2S)-4-(2,2-dimethyl-4,6-dioxo-1,3-dioxan-5-ylidene)-2-methyl-piperidine-1-carboxylate (21 g, 40%) LCMS m/z: 338.28 [M−1]⁻.

### Step 2: Preparation of tert-butyl (2S)-4-(3-chlorophenyl)-4-(2,2-dimethyl-4,6-dioxo-1, 3-dioxan-5-yl)-2-methyl-piperidine-1-carboxylate (C239)

To a stirred solution of magnesium (1.2 g, 0.0494 mol) in THF (250 mL) was added iodine (388 mg, 0.0787 mL, 0.0015 mol) and 1-bromo-3-chloro-benzene (4.2885 g, 7 mL, 0.0224 mol) at room temperature. Reaction mixture was heated to 85° C. and stirred at 85° C. for 2 hours. Then reaction mixture was allowed cool to room temperature. To the reaction mixture, tert-butyl (2S)-4-(2,2-dimethyl-4,6-dioxo-1,3-dioxan-5-ylidene)-2-methyl-piperidine-1-carboxylate (10 g, 0.0222 mol) and CuI (2 g, 0.0105 mol) were added. Reaction mixture was stirred at room temperature for 16 hours. Reaction mixture was cooled to 0° C., and quenched with saturated NH₄Cl solution (250 mL). It was then extracted with EtOAc (250 mL×3). Organic layers were washed with brine solution (150 mL), dried over anhydrous sodium sulfate, filtered, and concentrated to obtain tert-butyl (2S)-4-(3-chlorophenyl)-4-(2,2-dimethyl-4,6-dioxo-1,3-dioxan-5-yl)-2-methyl-piperidine-1-carboxylate (9 g, 30%). LCMS m/z 452.19 [M+1]⁺.

### Step 3: Preparation of 2-[(2S)-1-tert-butoxycarbonyl-4-(3-chlorophenyl)-2-methyl-4-piperidyl]acetic acid (C240)

To a stirred solution of tert-butyl (2S)-4-(3-chlorophenyl)-4-(2,2-dimethyl-4,6-dioxo-1,3-dioxan-5-yl)-2-methyl-piperidine-1-carboxylate (2.5 g, 0.0043 mol) in water (12 mL) in a Microwave Vial, was added 3-Pentanone (15 mL) at room temperature. Reaction mixture was heated to 140° C. (Under microwave condition) and maintained at 140° C. for 20 minutes. Reaction mixture was diluted with EtOAc (50 mL) and organic layer separated and concentrated to obtain 2-[(2S)-1-tert-butoxycarbonyl-4-(3-chlorophenyl)-2-methyl-4-piperidyl]acetic acid (1.9 g, 44%). LCMSMS m/z: 368.21 [M+1]⁺.

### Step 4: Preparation of tert-butyl (2S)-4-(2-chloro-2-oxo-ethyl)-4-(3-chlorophenyl)-2-methyl-piperidine-1-carboxylate (C241)

A stirred solution of 2-[(2S)-1-tert-butoxycarbonyl-4-(3-chlorophenyl)-2-methyl-4-piperidyl]acetic acid (2.25 g, 0.0043 mol) in MTBE (35 mL) was cooled to −10° C. Then DMF (94.400 mg, 0.1 mL, 0.0013 mol), Pyridine (586.80 mg, 0.6 mL, 0.0074 mol) and Oxalyl chloride (873.00 mg, 0.6 mL, 0.0069 mol) were added. After 20 minutes, reaction mixture was allowed to warm to room temperature, and stirred for 2 hours. Reaction mixture quenched in dry Methanol, then distilled under vacuum to provide tert-butyl (2S)-4-(2-chloro-2-oxo-ethyl)-4-(3-chlorophenyl)-2-methyl-piperidine-1-carboxylate (2.5 g, 90%).

### Step 5: Preparation of tert-butyl (2′S)-6-chloro-2′-methyl-3-oxo-spiro[indane-1,4′-piperidine]-1′-carboxylate (C242)

A stirred solution of AlCl₃ (62 mg, 464.97 μmol) in DCM (8 mL) was cooled to −30° C., then AlCl₃ (62 mg, 464.97 μmol) in DCM (8 mL) was added. Reaction mixture was stirred at −20° C. for 1 hour, then allowed to warm to room temperature and stirred for 1 hour. Reaction mixture poured into ice cold Water (10 mL) and stirred for 30 minutes. Aqueous layer was separated, and added into another flask containing MTBE (10 mL). This solution was cooled to −10° C., NaOH (10 M aq. solution) (0.1 mL of 10 M, 0.0010 mol) and Boc₂O (47.500 mg, 0.05 mL, 217.64 μmol) were added. Reaction mixture was allowed to room temperature and stirred for 16 hours. The reaction mixture was diluted with EtOAc (70 mL). Organic layer was separated and extracted with EtOAc (40 mL×3). Organic layer was dried over anhydrous sodium sulfate, filtered, and concentrated to provide a crude residue which was purified by reverse phase HPLC (Gradient: 0-20% MeCN in 10 mM ammonium bicarbonate) to provide tert-butyl (2′S)-6-chloro-2′-methyl-3-oxo-spiro[indane-1,4′-piperidine]-1′-carboxylate (9 mg, 16%). ¹H NMR (400 MHz, DMSO-d6) δ 7.676 (t, J=8.8 Hz, 2H), 7.475 (s, 2H), 7.388-7.356 (m, 2H), 4.62 (brs, 1H), 4.017-3.62 (m, 3H), 3.42-3.31 (m, 1H), 3.01-2.95 (m, 1H), 2.75 (d, J=2.4 Hz, 3H), 2.32-2.21 (m, 1H), 2.008 (s, 6H), 1.92-1.85 (m, 1H), 1.608 (s, 18H), 1.278-1.242 (m, 5H) LCMS m/z: 350.18 [M+1]⁺.

### Step 6: Preparation of tert-butyl (2′S)-6-chloro-3-hydroxy-2′-methyl-spiro[indane-1,4′-piperidine]-1′-carboxylate (C243)

A stirred solution of tert-butyl (2′S)-6-chloro-2′-methyl-3-oxo-spiro[indane-1,4′-piperidine]-1′-carboxylate (500 mg, 0.0014 mol) in MeOH (30 mL) was cooled to −10° C., NaBH₄ (200 mg, 0.2116 mL, 0.0053 mol) was added. After 20 minutes, reaction was slowly allowed to warm to room temperature and stirred for 4 hours. Reaction mixture was quenched with ice cold water (60 mL), then extracted with DCM (150 mL×3). Organic layer washed with brine solution (40 mL), dried over anhydrous sodium sulfate, filtered, and concentrated to afford tert-butyl (2′S)-6-chloro-3-hydroxy-2′-methyl-spiro[indane-1,4′-piperidine]-1′-carboxylate (450 mg, 87%) LCMS m/z 352.07 [M+1]⁺.

### Step 7: Preparation of (2′S)-5-chloro-2′-methyl-spiro[indane-3,4′-piperidine]-1-ol (C244)

A stirred solution of tert-butyl (2′S)-6-chloro-3-hydroxy-2′-methyl-spiro[indane-1,4′-piperidine]-1′-carboxylate (410 mg, 0.0011 mol) in DCM (15 mL) was cooled to 0° C., and TFA (2.2200 g, 1.5 mL, 0.0195 mol) was added. After 10 minutes, reaction was slowly allowed to room temperature and stirred for 4 hours. Reaction mixture was concentrated to afford (2′S)-5-chloro-2′-methyl-spiro[indane-3,4′-piperidine]-1-ol (Trifluoroacetic Acid Salt) (350 mg, 70%) LCMS m/z 252.17 [M+1]⁺.

### Step 8: Preparation of (2′S)-5-chloro-2′-methyl-1′-[[1-(2-methylsulfonylethyl)pyrazol-4-yl]methyl]spiro[indane-3,4′-piperidine]-1-ol (306) and (2′S)-5-chloro-2′-methyl-1′-[[1-(2-methylsulfonylethyl)pyrazol-4-yl]methyl]spiro[indane-3,4′-piperidine]-1-ol (307)

To a stirred solution of (2′S)-5-chloro-2′-methyl-spiro[indane-3,4′-piperidine]-1-ol (Trifluoroacetic Acid Salt) (350 mg, 773.25 μmol) in DMF (15 mL) were added K₂CO₃ (580 mg, 0.0042 mol), 4-(chloromethyl)-1-(2-methylsulfonylethyl)pyrazole (Hydrochloric Acid Salt) (200 mg, 655.98 μmol) and KI (120 mg, 722.88 μmol) at room temperature. Reaction mixture was stirred at room temperature for 16 hours. Reaction mixture was filtered through celite, and washed with EtOAc (20 mL). Filtrate was concentrated to obtained crude residue which was purified by chiral SFC chromatography (Column: Chiralcel OX-H (30×250 mm), 5p; Gradient: 40% (0.5% DEA in MeOH) in CO₂; Temperature 30° C.; Flow rate: 60.0 g/min) to provide:

First eluting peak: (2′S)-5-chloro-2′-methyl-1′-[[1-(2-methylsulfonylethyl)pyrazol-4-yl]methyl]spiro[indane-3,4′-piperidine]-1-ol 306 (6.1 mg, 2%). ¹H NMR (400 MHz, DMSO-d6) δ 7.722 (s, 1H), 7.408 (s, 1H), 7.300-7.223 (m, 3H), 5.32 (brs, 1H), 5.003-4.970 (m, 1H), 4.533-4.500 (m, 2H), 3.737-3.436 (m, 4H), 2.838-2.793 (m, 3H), 2.750-2.61 (m, 1H), 2.318-2.156 (m, 3H), 1.781-1.490 (m, 3H), 1.35-1.210 (m, 2H), 1.137-1.044 (m, 3H) LCMS m/z: 438.1 [M+1]⁺.

Second eluting peak: (2′S)-5-chloro-2′-methyl-1′-[[1-(2-methylsulfonylethyl)pyrazol-4-yl]methyl]spiro[indane-3,4′-piperidine]-1-ol 307 (4.3 mg, 1%). ¹H NMR (400 MHz, DMSO-d6) δ 7.725 (s, 1H), 7.410 (s, 1H), 7.300-7.223 (m, 3H), 5.32 (brs, 1H), 5.003-4.970 (m, 1H), 4.533-4.500 (m, 2H), 3.737-3.456 (m, 4H), 2.838-2.793 (m, 3H), 2.750-2.61 (m, 1H), 2.318-2.156 (m, 3H), 1.781-1.690 (m, 3H), 1.35-1.210 (m, 2H), 1.137-1.044 (m, 3H) LCMS m/z: 438.1 [M+1]⁺.

### Compound 308

### (2′S)-2′,5-dimethyl-1′-[[1-(2-methylsulfonylethyl)pyrazol-4-yl]methyl]spiro[indane-3,4′-piperidine]-1-ol (308)

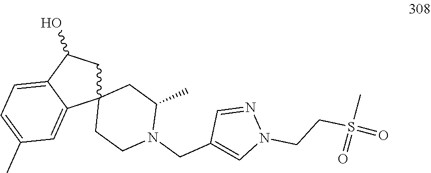 [SMILES: Cc1ccc2c(c1)C1(CCN(Cc3cnn(CCS(C)(=O)=O)c3)[C@@H](C)C1)CC2O]

Compound 308 was prepared in a similar manner to compound 306 and 307, using 1-bromo-3-methylbenzene. This provided (2′S)-2′,5-dimethyl-1′-[[1-(2-methylsulfonylethyl)pyrazol-4-yl]methyl]spiro[indane-3,4′-piperidine]-1-ol (4.9 mg). ¹H NMR (300 MHz, Chloroform-d) δ 8.40 (s, 1H), 8.00 (s, 1H), 7.78-7.72 (m, 1H), 7.27 (d, J=7.7 Hz, 1H), 7.11 (d, J=7.8 Hz, 1H), 7.05 (s, 1H), 5.14 (d, J=7.5 Hz, 1H), 4.72 (t, J=6.2 Hz, 2H), 4.50 (d, J=14.1 Hz, 1H), 4.29 (d, J=14.1 Hz, 1H), 3.72 (t, J=6.2 Hz, 2H), 3.44 (d, J=13.1 Hz, 1H), 3.07 (t, J=13.5 Hz, 1H), 2.90 (d, J=1.1 Hz, 3H), 2.48-2.31 (m, 4H), 2.26-1.80 (m, 4H), 1.67 (dd, J=23.2, 14.6 Hz, 1H), 1.57-1.47 (m, 3H). LCMS m/z: 438.1 [M+1]⁺.

### Compound 309

### (2′S)-2′,4-dimethyl-1′-((1-(2-(methylsulfonyl)ethyl)-1H-pyrazol-4-yl)methyl)-2, 3-dihydrospiro[indene-1,4′-piperidin]-3-ol (309)

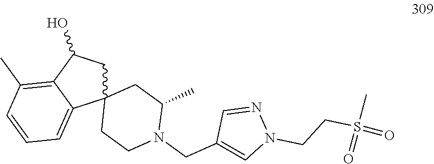 [SMILES: Cc1cccc2c1C(O)CC21CCN(Cc2cnn(CCS(C)(=O)=O)c2)[C@@H](C)C1]

Compound 309 was prepared in a similar manner to compound 306 and 307, using 1-bromo-3-methylbenzene. This provided (2′S)-2′,4-dimethyl-1′-((1-(2-(methylsulfonyl)ethyl)-1H-pyrazol-4-yl)methyl)-2,3-dihydrospiro[indene-1,4′-piperidin]-3-ol (6.6 mg). ¹H NMR (300 MHz, Chloroform-d) δ 8.41 (s, 1H), 8.00 (s, 1H), 7.74 (s, 1H), 7.24 (t, J=7.5 Hz, 1H), 7.07 (dd, J=7.4, 5.6 Hz, 2H), 5.28 (dd, J=6.9, 2.4 Hz, 1H), 4.71 (dd, J=6.9, 5.3 Hz, 2H), 4.50 (dd, J=14.2, 1.8 Hz, 1H), 4.29 (d, J=14.1 Hz, 1H), 3.72 (t, J=6.1 Hz, 2H), 3.44 (d, J=12.7 Hz, 1H), 3.31 (p, J=1.7 Hz, under MeOD, 1H), 3.19-2.96 (m, 1H), 2.90 (s, 3H), 2.39 (s, 3H), 2.30-1.90 (m, 4H), 1.73-1.45 (m, 4H). LCMS m/z: 438.3 [M+1]⁺.

### Compound 310

### 6-chloro-1′-[(1-methylpyrazol-4-yl)methyl]spiro[indane-1,4′-piperidine]

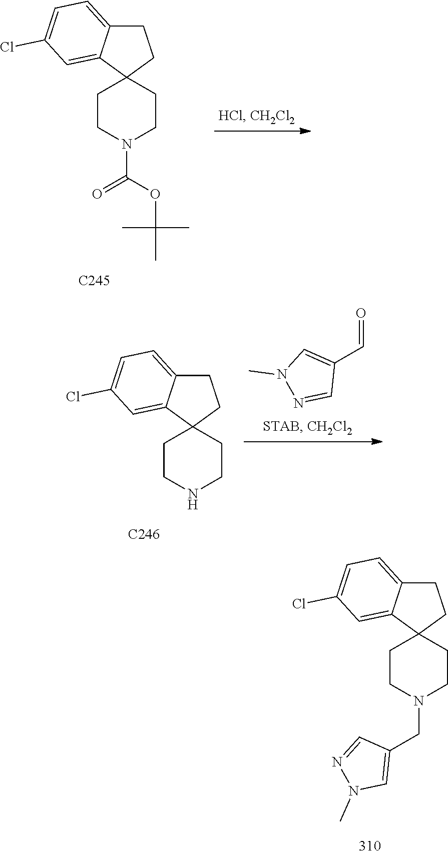

### Step 1: Preparation of 6-chlorospiro[indane-1,4′-piperidine](C246)

A solution of tert-butyl 6-chlorospiro[indane-1,4′-piperidine]-1′-carboxylate (50 mg, 0.1554 mmol) in DCM (2 mL) was treated with HCl (200 μL of 4 M, 0.8000 mmol) and the reaction was stirred at ambient temperature for 3 hours. The reaction was concentrated to afford 6-chlorospiro[indane-1,4′-piperidine](Hydrochloride salt) (37 mg, 88%). ¹H NMR (300 MHz, DMSO-d6) δ 8.81 (s, 2H), 7.30-7.20 (m, 2H), 7.11 (d, J=1.8 Hz, 1H), 3.29 (d, J=14.1 Hz, under water, 2H), 3.10-2.92 (m, 2H), 2.86 (t, J=7.3 Hz, 2H), 2.09 (t, J=7.3 Hz, 2H), 2.00 (td, J=13.7, 4.4 Hz, 2H), 1.63 (d, J=13.9 Hz, 2H). LCMS m/z: 222.06 [M+1]⁺.

### Step 2: Preparation of 6-chloro-1′-[(1-methylpyrazol-4-yl)methyl]spiro[indane-1,4′-piperidine](310)

A solution of 6-chlorospiro[indane-1,4′-piperidine](Hydrochloride salt) (28 mg, 0.1035 mmol) and 1-methylpyrazole-4-carbaldehyde (20 mg, 0.1816 mmol) in DCM (1.3 mL) were treated with STAB (70 mg, 0.3319 mmol). After 3 hours, reaction was quenched with saturated sodium bicarbonate solution and extracted with DCM (3×) through a phase separator. The organics were concentrated and purified by reverse phase HPLC (Gradient: 0-20% MeCN in aqueous 0.1% TFA) to provide 6-chloro-1′-[(1-methylpyrazol-4-yl)methyl]spiro[indane-1,4′-piperidine](Trifluoroacetate salt) (30.5 mg, 59%). ¹H NMR (300 MHz, DMSO-d6) δ 9.70 (s, 1H), 7.86 (s, 1H), 7.56 (d, J=0.7 Hz, 3H), 7.31-7.19 (m, 2H), 7.09 (d, J=1.7 Hz, 1H), 4.24 (d, J=4.6 Hz, 2H), 3.46-3.34 (m, 2H), 3.05 (q, J=11.8 Hz, 2H), 2.87 (t, J=7.2 Hz, 2H), 2.12-1.91 (m, 5H), 1.71 (d, J=14.2 Hz, 2H). LCMS m/z: 316.31 [M+1]⁺.

### Compound 311

### 6-chloro-1′-[(1-methylpyrazol-4-yl)methyl]spiro[isoindoline-1,4′-piperidine](311)

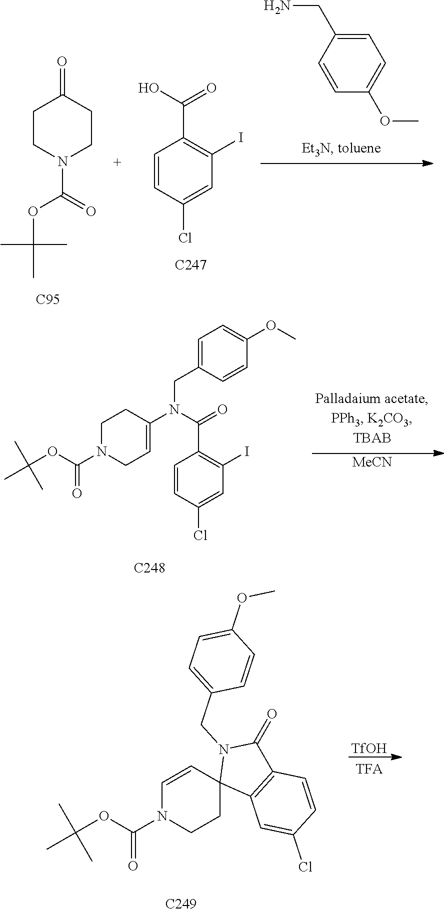

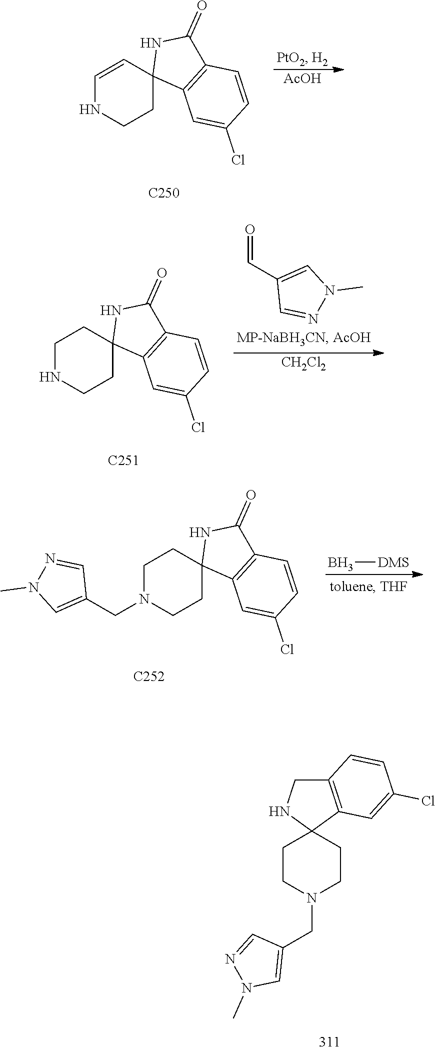

### Step 1: Preparation of tert-butyl 4-[(4-chloro-2-iodo-benzoyl)-[(4-methoxyphenyl)methyl]amino]-3,6-dihydro-2H-pyridine-1-carboxylate (C248)

A solution of tert-butyl 4-oxopiperidine-1-carboxylate (22 g, 110.42 mmol), (4-methoxyphenyl)methanamine (15.2 g, 110.80 mmol), and toluene (250 mL) was prepared. The mixture was heated to reflux overnight. After cooling to room temperature, the solvent was evaporated. The residue obtained was used directly in the next step without further purification. The crude residue was redissolved in toluene (500 mL) then 4-chloro-2-iodo-benzoic acid (25 g, 86.737 mmol) and triethylamine (14.520 g, 20 mL, 143.49 mmol) were added. The resulting solution was heated under reflux overnight. After cooling to room temperature, the mixture was added with 100 mL of 0.5 M aq. HCl solution and the layers were shaken and separated. The organic layer was washed with brine (200 mL), dried over anhydrous sodium sulfate, and concentrated. The crude residue was purified by flash column chromatography (Gradient: 0-40% EtOAc in hexanes) to provide tert-butyl 4-[(4-chloro-2-iodo-benzoyl)-[(4-methoxyphenyl)methyl]amino]-3,6-dihydro-2H-pyridine-1-carboxylate (41 g, 49%). LCMS m/z: 583.4 [M+1]⁺.

### Step 2: Preparation of tert-butyl 6′-chloro-2′-[(4-methoxyphenyl)methyl]-3′-oxo-spiro[2, 3-dihydropyridine-4,1′-isoindoline]-1-carboxylate (C249)

Tert-butyl 4-[(4-chloro-2-iodo-benzoyl)-[(4-methoxyphenyl)methyl]amino]-3,6-dihydro-2H-pyridine-1-carboxylate (23 g, 23.676 mmol) was dissolved in acetonitrile (300 mL) in a 500-mL round bottom flask fitted with a condenser and the mixture was purged with nitrogen gas for 1 hour. Palladium acetate (1.77 g, 7.8839 mmol), triphenylphosphine (4.14 g, 15.784 mmol), potassium carbonate (11 g, 79.591 mmol), and tetrabutylammonium bromide (12.7 g, 39.396 mmol) were added. The mixture was heated under reflux overnight. The mixture was cooled to room temperature and rotary evaporated. The residue was dissolved in EtOAc (100 mL) and water (100 mL). The mixture was shaken and the layers were separated. The organic layer was washed with brine (100 mL), dried over anhydrous sodium sulfate, and concentrated to give the crude product which was purified by flash column chromatography (Gradient: 0-40% EtOAc in hexanes) yielding tert-butyl 6′-chloro-2′-[(4-methoxyphenyl)methyl]-3′-oxo-spiro[2,3-dihydropyridine-4,1′-isoindoline]-1-carboxylate (6 g, 55%). LCMS m/z: 455.4 [M+1]⁺.

### Step 3: Preparation of 5′-chlorospiro[2,3-dihydro-1H-pyridine-4,3′-isoindoline]-1′-one (C250)

tert-butyl 6′-chloro-2′-[(4-methoxyphenyl)methyl]-3′-oxo-spiro[2,3-dihydropyridine-4,1′-isoindoline]-1-carboxylate (6 g, 12.925 mmol) in trifluoroacetic acid (65 mL) and trifluoromethanesulfonic acid (11.900 g, 7 mL, 79.293 mmol) was heated to 60° C. overnight. The solvent was removed by rotary evaporation and saturated aqueous sodium bicarbonate was added until solution was at pH=7. The aqueous solution was saturated with NaCl and was extracted with EtOAc (3×100 mL). The combined organic solutions were dried over anhydrous sodium sulfate, filtered, and rotary evaporated yielding crude 5′-chlorospiro[2,3-dihydro-1H-pyridine-4,3′-isoindoline]-1′-one (13 g). This was used in the next step without further purification. LCMS m/z: 235.2 [M+1]⁺.

### Step 4: Preparation of 5-chlorospiro[isoindoline-3,4′-piperidine]-1-one (C251)

Into a solution of crude 5′-chlorospiro[2,3-dihydro-1H-pyridine-4,3′-isoindoline]-1′-one (13 g, 43.207 mmol) in acetic acid (100 mL) was added PtO₂ (1 g, 4.4037 mmol), and the mixture was purged with nitrogen and evacuated and refilled with hydrogen. It was stirred under H₂ balloon overnight. The mixture was filtered and the filter cake was washed with acetic acid (3×50 mL). The combined filtrates were concentrated by rotary evaporation and the residue was added with saturated aqueous sodium bicarbonate solution until pH=7. It was saturated with sodium chloride and extracted with EtOAc (3×80 mL). The combined organic solutions were dried over anhydrous sodium sulfate, filtered, and rotary evaporated yielding crude 5-chlorospiro[isoindoline-3,4′-piperidine]-1-one (13 g). The crude was used in the next step without further purification. LCMS m/z: 237.2 [M+1]⁺.

### Step 5: Preparation of 5-chloro-1′-[(1-methylpyrazol-4-yl)methyl]spiro[isoindoline-3,4′-piperidine]-1-one (C252)

To a microwave vial containing 5-chlorospiro[isoindoline-3,4′-piperidine]-1-one (50 mg, 0.2112 mmol), 1-methylpyrazole-4-carbaldehyde (30 mg, 0.2724 mmol), dichloromethane (1 mL), and acetic acid (63.360 mg, 0.06 mL, 1.0551 mmol) was added MP-sodium cyanoborohydride (145 mg, 2 mmol/g, 0.2900 mmol). The vial was capped and heated to 110° C. for 30 min using a microwave reactor. After cooling, the solid phase reagent was filtered off and washed with 15 mL of dichloromethane. The combined filtrates were removed by rotary evaporation and the residue was purified by reverse phase HPLC (Gradient: 0-20% MeCN in aqueous 0.1% TFA) yielding 5-chloro-1′-[(1-methylpyrazol-4-yl)methyl]spiro[isoindoline-3,4′-piperidine]-1-one (Trifluoroacetic Acid Salt) (28.6 mg, 30%). ¹H NMR (500 MHz, DMSO-d6) δ 10.26 (s, 1H), 9.57 (s, 1H), 7.90 (s, 1H), 7.72-7.64 (m, 1H), 7.63-7.55 (m, 3H), 4.24-4.14 (m, 2H), 3.92-3.83 (m, 3H), 3.57-3.44 (m, 2H), 3.36-3.16 (m, 2H), 2.49-2.43 (m, 2H), 1.69 (d, J=14.0 Hz, 2H). LCMS m/z: 331.2 [M+1]⁺.

### Step 6: Preparation of 6-chloro-1′-[(1-methylpyrazol-4-yl)methyl]spiro[isoindoline-1,4′-piperidine](311)

Into a suspension of 5-chloro-1′-[(1-methylpyrazol-4-yl)methyl]spiro[isoindoline-3,4′-piperidine]-1-one (800 mg, 2.2974 mmol) in toluene (45 mL) was added BH₃-DMS (1.7 mL of 2 M, 3.4000 mmol) and the suspension was stirred at room temperature for 1 hour and at 110° C. (reflux) for 2 h. THF (6 mL) was added to help in dissolution of the starting material. Heating was continued overnight. After cooling to room temperature, MeOH (20 mL) was added and the solution was refluxed for 15 min. The solvents were removed by rotary evaporation. Purification by reverse phase HPLC (Gradient: 0-20% MeCN in aqueous 0.1% TFA) provided 6-chloro-1′-[(1-methylpyrazol-4-yl)methyl]spiro[isoindoline-1,4′-piperidine](Trifluoroacetic Acid (2)) 311 (27 mg, 2%). ¹H NMR (500 MHz, DMSO-d6) δ 7.89 (s, 1H), 7.59 (s, 1H), 7.50 (d, J=13.3 Hz, 2H), 7.30 (s, 1H), 6.51 (s, 2H), 4.72-4.52 (m, 1H), 4.34-4.21 (m, 1H), 3.88 (s, 3H), 3.25-3.11 (m, 2H), 2.41-2.18 (m, 3H). LCMS m/z: 317.1 [M+1]⁺.

### Compound 312

### 1-[5-chloro-1′-[[1-(2-methylsulfonylethyl)pyrazol-4-yl]methyl]spiro[indane-3,4′-piperidine]-1-yl]imidazolidin-2-one (312)

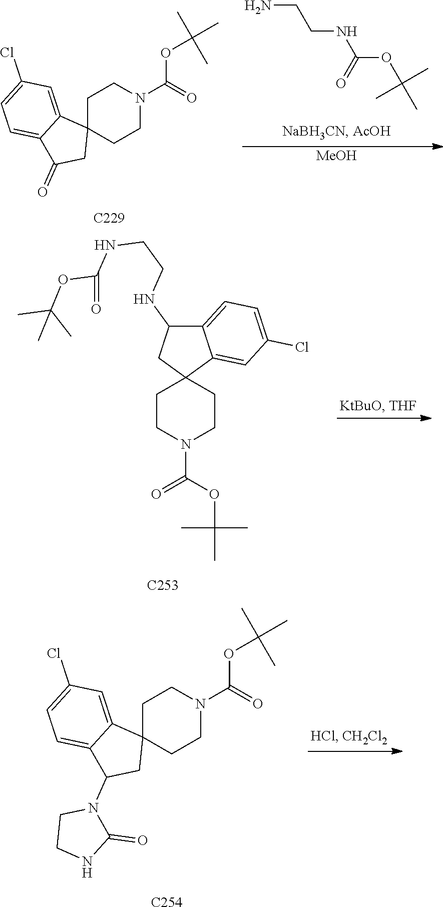

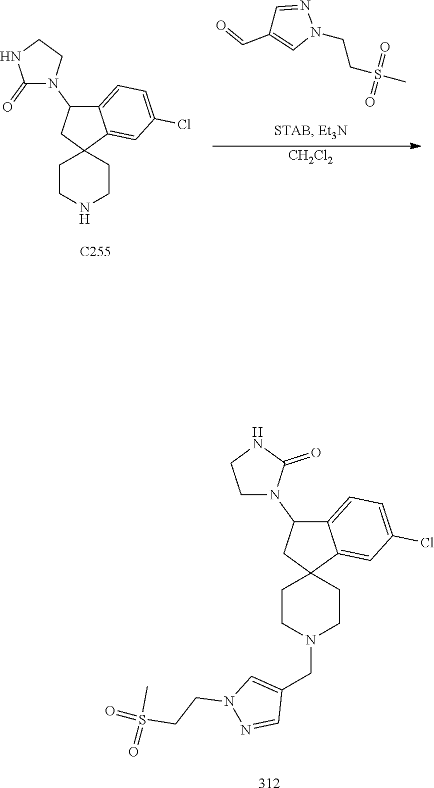 [SMILES: O=C1NCCN1C1CC2(CCNCC2)c2cc(Cl)ccc21 ; CS(=O)(=O)CCn1cc(C=O)cn1 ; "CH2Cl2" ; "C255" ; CS(=O)(=O)CCn1cc(CN2CCC3(CC2)CC(N2CCNC2=O)c2ccc(Cl)cc23)cn1 ; "312"]

### Step 1: Preparation of tert-butyl 3-[2-(tert-butoxycarbonylamino)ethylamino]-6-chloro-spiro[indane-1,4′-piperidine]-1′-carboxylate (C253)

Tert-butyl N-(2-aminoethyl)carbamate (650 mg, 4.057 mmol) and tert-butyl 6-chloro-3-oxo-spiro[indane-1,4′-piperidine]-1′-carboxylate (430 mg, 1.280 mmol) in MeOH (4 mL) were treated with NaBH₃CN (500 mg, 7.956 mmol) followed by AcOH (400 μL, 7.034 mmol) and heated to 65° C. After 2 hours, the reaction was cooled to room temperature, concentrated via rotovap, and the residue stirred with DCM and saturated sodium bicarbonate overnight. The reaction mixture was extracted with DCM (3×) with a phase separator. The organics were concentrated via rotovap and the material was purified via flash column chromatography (Gradient: 0-100% EtOAc in Heptane) to provide tert-butyl 3-[2-(tert-butoxycarbonylamino)ethylamino]-6-chloro-spiro[indane-1,4′-piperidine]-1′-carboxylate (500 mg, 79%). ¹H NMR (300 MHz, Chloroform-d) δ 7.28 (s, 1H), 7.22 (dd, J=8.1, 1.9 Hz, 1H), 7.14 (d, J=1.9 Hz, 1H), 4.96 (s, 1H), 4.27 (t, J=7.1 Hz, 1H), 4.14 (d, J=9.3 Hz, 2H), 3.28 (d, J=6.6 Hz, 2H), 3.06-2.77 (m, 4H), 2.58 (dd, J=12.9, 7.2 Hz, 1H), 1.96 (td, J=12.9, 4.5 Hz, 1H), 1.49 (d, J=10.5 Hz, 21H). LCMS m/z: 480.28 [M+1]⁺.

### Step 2: Preparation of tert-butyl 6-chloro-3-(2-oxoimidazolidin-1-yl)spiro[indane-1,4′-piperidine]-1′-carboxylate (C254)

Tert-butyl 3-[2-(tert-butoxycarbonylamino)ethylamino]-6-chloro-spiro[indane-1,4′-piperidine]-1′-carboxylate (500 mg, 1.011 mmol) in THF (8.5 mL) was treated with KtBuO (370 mg, 3.297 mmol) and heated to 60° C. for 30 minutes. The reaction was cooled to room temperature, diluted with water, and extracted with DCM (3×) through a phase separator. The organics were concentrated via rotovap to afford tert-butyl 6-chloro-3-(2-oxoimidazolidin-1-yl)spiro[indane-1,4′-piperidine]-1′-carboxylate (440 mg, 97%). ¹H NMR (300 MHz, Chloroform-d) δ 7.24 (dd, J=8.0, 1.9 Hz, 1H), 7.17 (d, J=1.9 Hz, 1H), 7.11 (dd, J=8.0, 1.0 Hz, 1H), 5.63 (t, J=8.5 Hz, 1H), 4.68 (s, 1H), 4.14 (s, 2H), 3.48 (td, J=8.1, 1.2 Hz, 2H), 3.29 (q, J=8.1 Hz, 1H), 3.23-3.09 (m, 1H), 2.89 (dd, J=30.1, 15.0 Hz, 2H), 2.58 (dd, J=13.1, 8.1 Hz, 1H), 2.04 (td, J=13.0, 4.5 Hz, 1H), 1.75 (dd, J=13.0, 8.9 Hz, 1H), 1.51 (s, 12H). LCMS m/z: 406.09 [M+1]⁺.

### Step 3: Preparation of 1-(5-chlorospiro[indane-3,4′-piperidine]-1-yl)imidazolidin-2-one (C255)

Tert-butyl 6-chloro-3-(2-oxoimidazolidin-1-yl)spiro[indane-1,4′-piperidine]-1′-carboxylate (441 mg, 0.9778 mmol) in DCM (8 mL) was treated with HCl (2.2 mL of 4 M, 8.800 mmol). The reaction was stirred at room temperature overnight. The reaction mixture was concentrated to afford 1-(5-chlorospiro[indane-3,4′-piperidine]-1-yl)imidazolidin-2-one (Dihydrochloride salt) (360 mg, 91%). ¹H NMR (300 MHz, DMSO-d6) δ 8.90 (s, 2H), 7.34 (dd, J=8.1, 2.0 Hz, 1H), 7.19 (d, J=2.0 Hz, 1H), 7.09 (dd, J=8.1, 1.0 Hz, 1H), 6.83-6.31 (m, 1H), 5.42 (t, J=8.5 Hz, 1H), 3.39-2.82 (m, 9H), 2.50 (p, J=1.9 Hz, under DMSO, 1H), 2.40-2.21 (m, 1H), 1.83 (dd, J=13.2, 9.1 Hz, 2H), 1.68 (d, J=13.8 Hz, 2H). LCMS m/z: 306.05 [M+1]⁺.

### Step 4: Preparation of 1-[5-chloro-1′-[[1-(2-methylsulfonylethyl)pyrazol-4-yl]methyl]spiro[indane-3,4′-piperidine]-1-yl]imidazolidin-2-one (312)

1-(5-chlorospiro[indane-3,4′-piperidine]-1-yl)imidazolidin-2-one (Dihydrochloride salt) (54.5 mg, 0.1353 mmol) and 1-(2-methylsulfonylethyl)pyrazole-4-carbaldehyde (37 mg, 0.1575 mmol) in DCM (2 mL) were treated with TEA (0.1 mL, 0.7175 mmol). STAB (95 mg, 0.4504 mmol) was then added and the reaction was stirred at room temperature for 2 hours. The reaction was quenched with saturated sodium bicarbonate and extracted with DCM (3×) with a phase separator. The organics were concentrated via rotovap and purified by reverse phase HPLC (Gradient: 0-20% MeCN in aqueous 0.1% TFA) to provide 1-[5-chloro-1′-[[1-(2-methylsulfonylethyl)pyrazol-4-yl]methyl]spiro[indane-3,4′-piperidine]-1-yl]imidazolidin-2-one (Trifluoroacetate salt) 312 (93.8 mg, 93%). ¹H NMR (300 MHz, Chloroform-d) δ 7.97 (d, J=0.8 Hz, 1H), 7.71 (d, J=0.8 Hz, 1H), 7.31 (dd, J=8.0, 1.9 Hz, 1H), 7.26 (d, J=1.9 Hz, 1H), 7.15 (dd, J=8.0, 1.0 Hz, 1H), 5.51 (t, J=8.4 Hz, 1H), 4.76-4.64 (m, 2H), 4.31 (s, 2H), 3.71 (t, J=6.2 Hz, 2H), 3.62-3.48 (m, 2H), 3.47-3.32 (m, 3H), 3.28-3.01 (m, 3H), 2.90 (s, 3H), 2.57 (dd, J=13.4, 8.2 Hz, 1H), 2.35 (td, J=14.1, 4.0 Hz, 1H), 1.89 (dd, J=9.7, 6.6 Hz, 5H). LCMS m/z: 492.36 [M+1]⁺.

### Compound 313

### 6-chloro-3-(difluoromethylene)-1′-[[1-(2-methylsulfonylethyl)pyrazol-4-yl]methyl]spiro[indane-1,4′-piperidine](313)

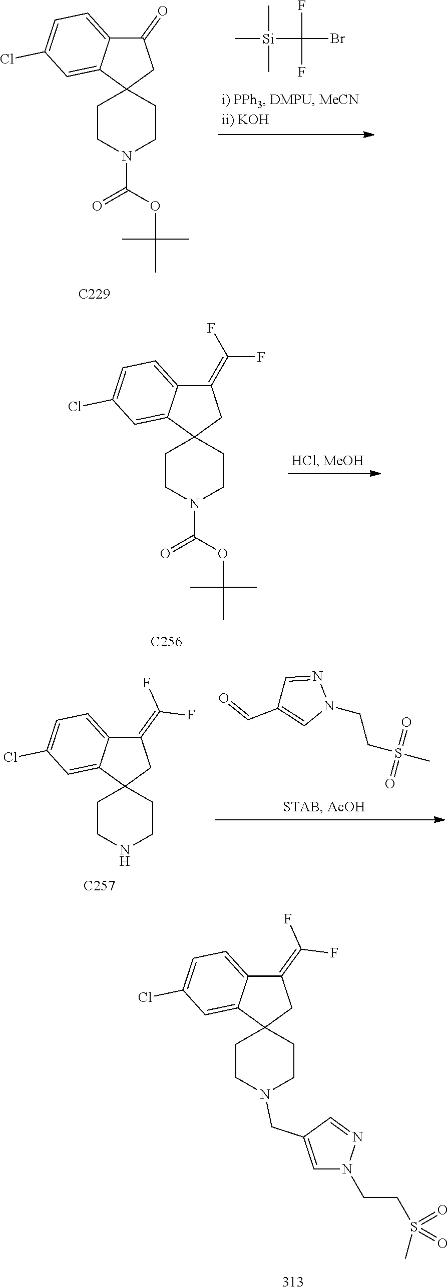

### Step 1: Preparation of tert-butyl 6-chloro-3-(difluoromethylene)spiro[indane-1,4′-piperidine]-1′-carboxylate (C256)

To a solution of tert-butyl 6-chloro-3-oxo-spiro[indane-1,4′-piperidine]-1′-carboxylate (518 mg, 1.542 mmol) in MeCN (6 mL) was added DMPU (372 μL, 3.088 mmol) followed by [bromo(difluoro)methyl]-trimethyl-silane (564 mg, 2.777 mmol), followed by PPh₃ (486 mg, 1.853 mmol). Reaction was stirred at 60° C. for 30 minutes. Solution was brought to room temperature and added KOH (8 mL of 1 M, 8.000 mmol), followed by stirring overnight. The reaction was diluted further with water and extracted with EtOAc. Organic layer was concentrated and purified by flash column chromatography (Gradient: 0-50% EtOAc in hepatanes) to provide tert-butyl 6-chloro-3-(difluoromethylene)spiro[indane-1,4′-piperidine]-1′-carboxylate (18.5 mg, 3%) ¹H NMR (300 MHz, Chloroform-d) δ 7.27 (dd, J=8.3, 1.1 Hz, 1H), 7.15 (dd, J=8.2, 2.0 Hz, 1H), 7.06 (d, J=2.0 Hz, 1H), 4.07 (s, 2H), 2.80 (t, J=13.3 Hz, 2H), 2.65 (t, J=3.2 Hz, 2H), 1.71 (td, J=13.2, 4.7 Hz, 2H), 1.46 (d, J=2.6 Hz, 2H), 1.42 (s, 9H). ¹⁹F NMR (282 MHz, Chloroform-d) δ− 86.64 (d, J=45.6 Hz), -87.02 (d, J=45.9 Hz). LCMS m/z 371.09 [M+1]⁺.

### Step 2: Preparation of 6-chloro-3-(difluoromethylene)spiro[indane-1,4′-piperidine](C257)

To tert-butyl 6-chloro-3-(difluoromethylene)spiro[indane-1,4′-piperidine]-1′-carboxylate (19 mg, 0.05059 mmol) in 1 mL methanol was added 1 mL 4 M HCl. Solution was heated to 50° C. for 20 minutes. Solution was evaporated to provide 6-chloro-3-(difluoromethylene)spiro[indane-1,4′-piperidine](Hydrochloride salt) (16 mg, 100%) ¹H NMR (300 MHz, Chloroform-d) δ 7.41 (dt, J=8.0, 1.1 Hz, 1H), 7.36-7.28 (m, 2H), 3.52-3.39 (m, 2H), 3.28-3.10 (m, 2H), 2.90 (t, J=3.2 Hz, 2H), 2.10 (td, J=14.1, 4.4 Hz, 2H), 1.87-1.70 (m, 2H). ¹⁹F NMR (282 MHz, Chloroform-d) δ− 88.90 (d, J=47.5 Hz), -89.33 (d, J=47.2 Hz).

### Step 3: Preparation of 6-chloro-3-(difluoromethylene)-1′-[[1-(2-methylsulfonylethyl)pyrazol-4-yl]methyl]spiro[indane-1,4′-piperidine](313)

To 6-chloro-3-(difluoromethylene)spiro[indane-1,4′-piperidine](Hydrochloride salt) (12 mg, 0.03919 mmol), 1-(2-methylsulfonylethyl)pyrazole-4-carbaldehyde (20 mg, 0.08514 mmol) HOAc (20 μL, 0.3517 mmol)) in 4 mL DCE, was added STAB (40 mg, 0.1896 mmol), and stirred at ambient temperature for 16 hours. Reaction mixture was concentrated and purified by reverse phase HPLC (Gradient: 0-20% MeCN in aqueous 0.1% FA) to provide 6-chloro-3-(difluoromethylene)-1′-[[1-(2-methylsulfonylethyl)pyrazol-4-yl]methyl]spiro[indane-1,4′-piperidine](Formic Acid Salt) 313 (10.1 mg, 51%) LCMS m/z: 456.15 [M+1]⁺.

### Compound 314

### 1-[5-chloro-1′-[(1-methylpyrazol-4-yl)methyl]spiro[indoline-3,4′-piperidine]-1-yl]ethenone (314)

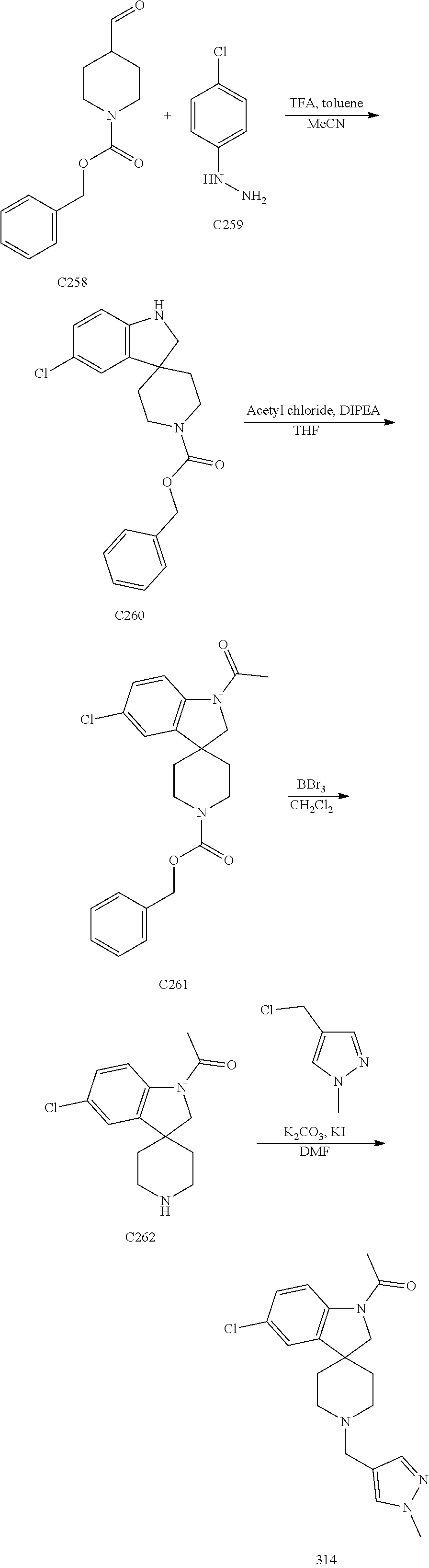

### Step 1: Preparation of benzyl 5-chlorospiro[indoline-3,4′-piperidine]-1′-carboxylate (C260)

To a stirred solution of (4-chlorophenyl)hydrazine (3.1654 g, 0.0222 mol) in toluene (122.5 mL) and MeCN (2.5 mL) was added TFA (7.6053 g, 5.1387 mL, 0.0667 mol) which was degassed for 15 min with argon. A solution of benzyl 4-formylpiperidine-1-carboxylate (5 g, 0.0202 mol) in toluene (4.9 mL) and MeCN (0.1 mL) was slowly added to above reaction mixture for 20 min. The reaction mixture was stirred at room temperature for 16 hours. The reaction mixture was evaporated to afford benzyl 5-chlorospiro[indoline-3,4′-piperidine]-1′-carboxylate (10 g, 30%), LCMS m/z: 355.38 [M+1]⁺. The crude product was moved forward to next step without purification.

### Step 2: Preparation of benzyl I-acetyl-5-chloro-spiro[indoline-3,4′-piperidine]-1′-carboxylate (C261)

To a stirred solution of benzyl 5-chlorospiro[indoline-3,4′-piperidine]-1′-carboxylate (250 mg, 574.48 μmol) in THF (5 mL) was added DIPEA (374.80 mg, 0.5051 mL, 0.0029 mol) and acetyl chloride (133.45 mg, 0.1209 mL, 0.0017 mol) at 0° C. The reaction mixture was stirred at room temperature for 3 hours. The reaction mixture was diluted with EtOAc (50 mL) and washed with saturated NaHCO₃ solution (50 mL). The organic layer was dried over sodium sulfate, filtered, and evaporated under vacuum to afford benzyl 1-acetyl-5-chloro-spiro[indoline-3,4′-piperidine]-1′-carboxylate (270 mg, 112%) ¹H NMR (400 MHz, CDCl₃) δ: 8.14 (d, J=8.8 Hz, 1H), 7.39-7.33 (m, 5H), 7.18 (dd, J=8.8, 2.4 Hz, 1H), 7.05 (d, J=2.0 Hz, 1H), 5.17 (s, 2H), 4.23 (brs, 2H), 3.92 (s, 2H), 2.95-2.94 (brs 2H), 2.25 (s, 3H), 1.83-1.81 (m, 2H), 1.70-1.67 (m, 2H) LCMS m/z: 399.19 [M+1]⁺. The crude product was moved forward to next step without purification.

### Step 3: Preparation of 1-(5-chlorospiro[indoline-3,4′-piperidine]-1-yl)ethenone (C262)

To a stirred solution of benzyl 1-acetyl-5-chloro-spiro[indoline-3,4′-piperidine]-1′-carboxylate (110 mg, 264.74 μmol) in DCM (5 mL) was added boron tribromide in DCM (0.7942 mL of 1 M, 794.20 μmol) dropwise at 0° C. The reaction mixture was allowed to stir at room temperature for 16 hours. The reaction was evaporated under vacuum and washed with diethyl ether, and dried under vacuum to afford 1-(5-chlorospiro[indoline-3,4′-piperidine]-1-yl)ethanone (Bromide Ion (1)) (130 mg). ¹H NMR (300 MHz, DMSO-d6) δ: 8.05 (d, J=8.4 Hz, 1H), 7.28 (dd, J=8.7, 2.1 Hz, 1H), 7.16 (d, J=2.1 Hz, 1H), 4.11 (s, 2H), 3.41-3.32 (m, 2H), 3.13-3.0 (m, 2H), 2.21 (s, 3H), 2.04-1.80 (m, 4H), 1.09 (t, J=6.9 Hz, 1H) LCMS m/z: 265.15 [M+1]⁺.

### Step 4: Preparation of 1-[5-chloro-1′-[(1-methylpyrazol-4-yl)methyl]spiro[indoline-3,4′-piperidine]-1-yl]ethenone (314)

To a stirred solution of 1-(5-chlorospiro[indoline-3,4′-piperidine]-1-yl)ethanone (hydrobromic acid salt) (137 mg, 380.49 μmol) in DMF (5 mL) was added K₂CO₃ (263 mg, 0.0019 mol), KI (13 mg, 78.312 μmol) and 4-(chloromethyl)-1-methyl-pyrazole (hydrochloric acid salt) (64 mg, 363.99 μmol) at room temperature. The reaction mixture was stirred at 100° C. for 16 hours. The reaction mixture was diluted with EtOAc (50 mL) and washed with water (20 mL). The organic layer dried over sodium sulfate, filtered and concentrated. Purification by reverse phase HPLC (Gradient: 0-20% MeCN in aqueous 0.1% FA) provided 1-[5-chloro-1′-[(1-methylpyrazol-4-yl)methyl]spiro[indoline-3,4′-piperidine]-1-yl]ethanone (formic acid salt) 314 (8.8 mg, 5%) ¹H NMR (400 MHz, DMSO-d6) δ: 8.22 (s, 1H), 8.01 (d, J=8.4 Hz, 1H), 7.56 (s, 1H), 7.31 (d, J=2.4 Hz, 2H), 7.19 (dd, J=8.4, 2.4 Hz, 1H), 3.94 (s, 2H), 3.79 (s, 3H), 3.35 (s, 2H), 2.81 (d, J=11.6 Hz, 2H), 2.18 (s, 3H), 2.01 (t, J=11.6 Hz, 2H), 1.85-1.78 (m, 2H), 1.58 (d, J=12.0 Hz, 2H) LCMS m/z: 359.1 [M+1]⁺.

### Preparation C195 and C196

### (S)-1-((2,2,5,5-tetramethyl-1, 3-dioxolan-4-yl)methyl)-1H-pyrazole-4-carbaldehyde (C195) and (R)-1-((2,2,5,5-tetramethyl-1,3-dioxolan-4-yl)methyl)-1H-pyrazole-4-carbaldehyde (C196)

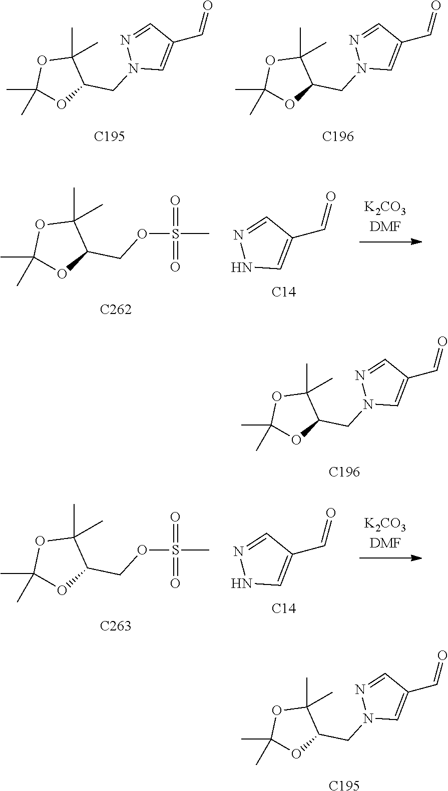

(S)-1-((2,2,5,5-tetramethyl-1,3-dioxolan-4-yl)methyl)-1H-pyrazole-4-carbaldehyde (C195) and (R)-1-((2,2,5,5-tetramethyl-1,3-dioxolan-4-yl)methyl)-1H-pyrazole-4-carbaldehyde (C196) could be prepared by someone skilled in the art of organic synthesis using similar procedure as demonstrated in the preparation of S39 using starting materials C14, C262 and C263.

### Example 2. Assays for Detecting and Measuring APOL1 Inhibitor Properties of Compounds

### MultiTox-Fluor Multiplex Cytotoxicity Assay

The MultiTox-Fluor Multiplex Cytotoxicity Assay is a single-reagent-addition, homogeneous, fluorescence assay that measures the number of live and dead cells simultaneously in culture wells. The assay measures cell viability and cytotoxicity by detecting two distinct protease activities. The live-cell protease activity is restricted to intact viable cells and is measured using a fluorogenic, cell-permeant peptide glycyl-phenylalanylamino fluorocoumarin (GF-AFC) substrate. The substrate enters intact cells, where it is cleaved to generate a fluorescent signal proportional to the number of living cells. This live-cell protease activity marker becomes inactive upon loss of membrane integrity and leakage into the surrounding culture medium. A second, cell-impermeant, fluorogenic peptide substrate (bis-AAF-R110 Substrate) is used to measure dead-cell protease that has been released from cells that have lost membrane integrity. A ratio of dead to live cells is used to normalize data.

Briefly, the tet-inducible transgenic APOL1 T-REx-HEK293 cell lines were incubated with 50 ng/mL tet to induce APOL1 in the presence of 3-(2-(4-fluorophenyl)-1H-indol-3-yl)-N-((3S,4R)-4-hydroxy-2-oxopyrrolidin-3-yl)propenamide at 10.03, 3.24, 1.13, 0.356, 0.129, 0.042, 0.129, 0.0045, 0.0015, 0.0005 μM in duplicate for 24 hours in a humidified 37° C. incubator. The MultiTox reagent was added to each well and placed back in the incubator for an additional 30 minutes. The plate was read on the EnVision plate reader. A ratio of dead to live cells was used to normalize, and data was imported, analyzed, and fit using Genedata Screener (Basel, Switzerland) software. Data was normalized using percent of control, no tet (100% viability), and 50 ng/mL tet treated (0% viability),and fit using Smart Fit. The reagents, methods, and complete protocol for the MultiTox assay are described below.

TABLE 19
Reagents Used in the Multi-Tox Assay

| Reagent | Catalog Number | Vendor |
|---|---|---|
| 384 well, transparent, flat | 356663 | Corning |
| bottom tissue culture | | (Corning, NY) |
| treated, Poly-D lysine | | |
| coated | | |
| 384 well round bottom | 3656 | CoStar |
| polypropylene plates | | (Corning, NY) |
| Universal plate lids | 250002 | Thermo Fisher |
| | | (Waltham) |
| Axygen 30 μL tips for | VT-384-31UL-R-S | Corning |
| Bravo 384 well | | (Corning, NY) |
| MultiTox-Fluor Multiplex | G9202 | Promega |
| Cytotoxicity Assay | | (Madison, WI) |
| 225 cm² flask, angled neck, | 431082 | Corning |
| treated, vented cap | | (Corning, NY) |
| Dulbecco's Phosphate- | 14190-136 | Thermo Fisher |
| Buffered Saline (DPBS), | | (Waltham) |
| calcium and magnesium- | | |
| free | | |
| Dulbecco's Modified Eagle | 11960-077 | Thermo Fisher |
| Medium (DMEM), high | | (Waltham) |
| glucose, no glutamine, no | | |
| sodium pyruvate | | |
| Fetal Bovine Serum (FBS), | 631368 | Takara (Kusatsu, |
| tetracycline-free, US- | | Japan) |
| Sourced | | |
| L-Glutamine, 200 mM | 25030-081 | Thermo Fisher |
| | | (Waltham) |
| Penicillin-Streptomycin, | 15140-122 | Thermo Fisher |
| 10,000 Units/mL | | (Waltham) |
| Blasticidin S HCl, 10 | A11139-03 | Thermo Fisher |
| mg/mL | | (Waltham) |
| Tetracycline hydrochloride | T7660-5G | Sigma (St. Louis, MO) |
| Puromycin | A11138-03 | Thermo Fisher |
| dihydrochloride, 10 mg/mL | | (Waltham) |
| Trypsin-EDTA | 25300-054 | Thermo Fisher |
| | | (Waltham) |

TABLE 20
Equipment Used in the Multi-Tox Assay

| Instrument | Model | Supplier | Location |
|---|---|---|---|
| Bravo | 16050-101 | Agilent Technologies | Santa Clara, CA |
| Multidrop Combi | N/A | Thermo Scientific | Waltham, MA |
| EnVision | N/A | PerkinElmer | Waltham, MA |

### Multi-Tox Assay Protocol

Human embryonic kidney (HEK293) cell lines containing a tet-inducible expression system (T-REx™; Invitrogen, Carlsbad, CA) and Adeno-associated virus site 1 pAAVS1-Puro-APOL1 G0 or pAAVS1-Puro-APOL1 G1 or pAAVS1-Puro-APOL1 G2 Clones G0 DC2.13, G1 DC3.25, and G2 DC4.44 were grown in a T-225 flask at ˜90% confluency in cell growth media (DMEM, 10% Tet-free FBS, 2 mM L-glutamine, 100 Units/mL penicillin-streptomycin, 5 μg/mL blasticidin S HCl, 1 μg/mL puromycin dihydrochloride). Cells were washed with DPBS and then trypsinized to dissociate from the flask. Media was used to quench the trypsin, cells were then pelleted at 200 g and resuspended in fresh cell assay media (DMEM, 2% Tet-free FBS, 2 mM L-glutamine, 100 Units/mL penicillin-streptomycin). Cells were counted and diluted to 1.17×10⁶ cells/mL. 20 μL of cells (23,400/well) were dispensed in every well of a 384-well Poly-D-Lysine coated plate using the Multidrop dispenser. The plates were then incubated at room temperature for one hour.

Tetracycline is needed to induce APOL1 expression. 1 mg/mL tet stock in water was diluted to 250 ng/mL (5×) in cell assay media. 60 μL of cell assay media (no tet control) was dispensed in columns 1 and 24, and 60 μL of 5× tet in 384-PP-round bottom plate was dispensed in columns 2 to 23 with the Multidrop dispenser.

Assay ready plates from the Global Compound Archive were ordered using template 384_APOL1Cell_DR10n2_50 uM_v3. Compounds were dispensed at 200 nL in DMSO. The final top concentration was 10 μM with a 10 point 3-fold dilution in duplicate in the MultiTox assay.

20 μL was transferred from the 5× tet plate to the ARP and mixed, then 5 μL of 5× tet and the compounds were transferred to the cell plate and mixed using the Bravo. The cell plate was placed in the humidified 37° C. 5% CO₂ incubator for 24 hours.

The MultiTox-Fluor Multiplex Cytotoxicity Assay was performed in accordance with the manufacturer's protocol. After cells were incubated with tet and compound for 24 hours, 25 μL of 1× MultiTox reagent was added to each well using the Multidrop dispenser; the plates were placed on a plate shaker (600 rpm) for 2 minutes, then centrifuged briefly and placed back in the 37° C. incubator for 30 minutes. The cell viability (excitation: 400 nm, emission: 486 nm) and cytotoxicity (excitation: 485 nm, emission: 535 nm) were read using the EnVision plate reader. A ratio of dead (cytotoxicity) to live (viability) cells was reported. Data was exported and analyzed in Genedata. Data was normalized using percent of control, no tet (100% viability), and 50 ng/mL tet treated (0% viability), and fit using Smart Fit settings in Genedata.

### Potency Data for Compounds 1 to 314

The compounds of Formula I are useful as inhibitors of APOL1 activity. Table 20 below illustrates the IC₅₀ of Compounds 1 to 299 using procedures described above. The procedures above may also be used to determine the potency of any compounds of Formula I. In Table below, the following meanings apply. For IP₅₀ (i.e., IC₅₀ for cell proliferation), “+++” means≤50 nM; “++” means between 50 nM and 500 nM; “+” means≥500 nM. N.D.=Not determined.

TABLE 20
Potency Data for Compounds 1 to 314

| Compound No. | IP₅₀ |
|---|---|
| 1 | ++ |
| 2 | +++ |
| 3 | ++ |
| 4 | + |
| 5 | + |
| 6 | ++ |
| 7 | ++ |
| 8 | ++ |
| 9 | ++ |
| 10 | ++ |
| 11 | ++ |
| 12 | ++ |
| 13 | ++ |
| 14 | + |
| 15 | + |
| 16 | ND |
| 17 | + |
| 18 | ND |
| 19 | + |
| 20 | + |
| 21 | + |
| 22 | ++ |
| 23 | ++ |
| 24 | + |
| 25 | + |
| 26 | + |
| 27 | + |
| 28 | + |
| 29 | +++ |
| 30 | + |
| 31 | + |
| 32 | + |
| 33 | ++ |
| 34 | + |
| 35 | + |
| 36 | ++ |
| 37 | ++ |
| 38 | ++ |
| 39 | ++ |
| 40 | ++ |
| 41 | ++ |
| 42 | ND |
| 43 | + |
| 44 | + |
| 45 | ++ |
| 46 | ++ |
| 47 | ND |
| 48 | ++ |
| 49 | ++ |
| 50 | ++ |
| 51 | + |
| 52 | +++ |
| 53 | ++ |
| 54 | ++ |
| 55 | + |
| 56 | ++ |
| 57 | + |
| 58 | ND |
| 59 | + |
| 60 | + |
| 61 | + |
| 62 | ND |
| 63 | ND |
| 64 | ND |
| 65 | + |
| 66 | + |
| 67 | + |
| 68 | ND |
| 69 | + |
| 70 | + |
| 71 | + |
| 72 | + |
| 73 | + |
| 74 | + |
| 75 | ++ |
| 76 | +++ |
| 77 | +++ |
| 78 | +++ |
| 79 | ++ |
| 80 | +++ |
| 81 | ++ |
| 82 | + |
| 83 | + |
| 84 | + |
| 85 | + |
| 86 | + |
| 87 | + |
| 88 | +++ |
| 89 | + |
| 90 | +++ |
| 91 | +++ |
| 92 | +++ |
| 93 | +++ |
| 94 | +++ |
| 95 | + |
| 96 | +++ |
| 97 | + |
| 98 | +++ |
| 99 | + |
| 100 | +++ |
| 101 | ++ |
| 102 | ++ |
| 103 | +++ |
| 104 | +++ |
| 105 | +++ |
| 106 | +++ |
| 107 | +++ |
| 108 | + |
| 109 | +++ |
| 110 | ++ |
| 111 | +++ |
| 112 | +++ |
| 113 | + |
| 114 | +++ |
| 115 | +++ |
| 116 | +++ |
| 117 | +++ |
| 118 | +++ |
| 119 | +++ |
| 120 | ++ |
| 121 | +++ |
| 122 | +++ |
| 123 | +++ |
| 124 | +++ |
| 125 | +++ |
| 126 | +++ |
| 127 | + |
| 128 | + |
| 129 | + |
| 130 | ++ |
| 131 | +++ |
| 132 | +++ |
| 133 | +++ |
| 134 | +++ |
| 135 | +++ |
| 136 | ++ |
| 137 | + |
| 138 | + |
| 139 | + |
| 140 | + |
| 141 | +++ |
| 142 | ++ |
| 143 | +++ |
| 144 | +++ |
| 145 | ++ |
| 146 | +++ |
| 147 | +++ |
| 148 | +++ |
| 149 | +++ |
| 150 | +++ |
| 151 | + |
| 152 | +++ |
| 153 | + |
| 154 | ++ |
| 155 | ++ |
| 156 | +++ |
| 157 | +++ |
| 158 | +++ |
| 159 | ++ |
| 160 | ++ |
| 161 | +++ |
| 162 | ++ |
| 163 | ++ |
| 164 | ++ |
| 165 | +++ |
| 166 | ++ |
| 167 | +++ |
| 168 | ++ |
| 169 | +++ |
| 170 | +++ |
| 171 | +++ |
| 172 | +++ |
| 173 | +++ |
| 174 | ++ |
| 175 | +++ |
| 176 | ++ |
| 177 | ++ |
| 178 | ++ |
| 179 | +++ |
| 180 | ++ |
| 181 | +++ |
| 182 | +++ |
| 183 | ++ |
| 184 | +++ |
| 185 | ++ |
| 186 | +++ |
| 187 | +++ |
| 188 | +++ |
| 189 | +++ |
| 190 | + |
| 191 | +++ |
| 192 | + |
| 193 | ++ |
| 194 | +++ |
| 195 | +++ |
| 196 | +++ |
| 197 | +++ |
| 198 | +++ |
| 199 | ++ |
| 200 | ++ |
| 201 | +++ |
| 202 | +++ |
| 203 | +++ |
| 204 | ++ |
| 205 | +++ |
| 206 | ++ |
| 207 | + |
| 208 | + |
| 209 | +++ |
| 210 | ++ |
| 211 | +++ |
| 212 | +++ |
| 213 | ++ |
| 214 | +++ |
| 215 | + |
| 216 | +++ |
| 217 | ++ |
| 218 | + |
| 219 | +++ |
| 220 | + |
| 221 | + |
| 222 | + |
| 223 | ++ |
| 224 | + |
| 225 | +++ |
| 226 | +++ |
| 227 | +++ |
| 228 | ++ |
| 229 | + |
| 230 | +++ |
| 231 | ++ |
| 232 | +++ |
| 233 | +++ |
| 234 | +++ |
| 235 | +++ |
| 236 | ++ |
| 237 | ++ |
| 238 | + |
| 239 | +++ |
| 240 | + |
| 241 | + |
| 242 | ++ |
| 243 | ++ |
| 244 | +++ |
| 245 | ++ |
| 246 | +++ |
| 247 | + |
| 248 | +++ |
| 249 | + |
| 250 | ++ |
| 251 | ++ |
| 252 | + |
| 253 | ++ |
| 254 | + |
| 255 | + |
| 256 | ++ |
| 257 | ++ |
| 258 | + |
| 259 | + |
| 260 | ++ |
| 261 | ++ |
| 262 | + |
| 263 | + |
| 264 | + |
| 265 | + |
| 266 | + |
| 267 | + |
| 268 | + |
| 269 | + |
| 270 | + |
| 271 | + |
| 272 | + |
| 273 | + |
| 274 | + |
| 275 | + |
| 276 | + |
| 277 | +++ |
| 278 | +++ |
| 279 | +++ |
| 280 | +++ |
| 281 | +++ |
| 282 | +++ |
| 283 | ++ |
| 284 | ++ |
| 285 | ++ |
| 286 | + |
| 287 | ND |
| 288 | ++ |
| 289 | + |
| 290 | + |
| 291 | + |
| 292 | +++ |
| 293 | ++ |
| 294 | + |
| 295 | +++ |
| 296 | + |
| 297 | + |
| 298 | + |
| 299 | + |
| 300 | ND |
| 301 | ND |
| 302 | ++ |
| 303 | + |
| 304 | + |
| 305 | + |
| 306 | +++ |
| 307 | +++ |
| 308 | +++ |
| 309 | ++ |
| 310 | + |
| 311 | + |
| 312 | + |
| 313 | ND |
| 314 | + |

### OTHER EMBODIMENTS

This disclosure provides merely non-limiting example embodiments of the disclosed subject matter. One skilled in the art will readily recognize from the disclosure and claims, that various changes, modifications and variations can be made therein without departing from the spirit and scope of the disclosure as defined in the following claims.

1. A compound represented by the following structural formula: 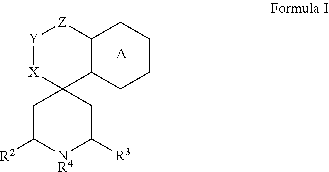
a tautomer thereof, a deuterated derivative of that compound or tautomer, or a pharmaceutically acceptable salt of any of the foregoing, wherein:
Ring A is chosen from 6-membered aryl and 6-membered heteroaryl groups, wherein the 6-membered aryl or 6-membered heteroaryl group is optionally substituted by 1, 2, 3, or 4 R¹ groups;
X is chosen from —CR^{1a}R^{1b}—, —C(O)—, —S—, —S(O)₂—, —NR^{1c}—, and —O—;
Y is chosen from —CR^{1a}R^{1b}—, —C(O)—, —S(O)₂—, —NR^{1c}—, and —O—;
Z is chosen from a bond, —CR^{1a}R^{1b}—, —NR^{1c}—, —C(O)—, —S(O)₂—, and —O—, wherein:
at least one of X and Y is chosen from —CR^{1a}R^{1b}— and —C(O)—;
R^{1a}, R^{1b}, and R^{1c}, for each occurrence, are each independently chosen from hydrogen and R¹ groups;
R¹, for each occurrence, is independently chosen from halogen, —OH, cyano, phenyl, C₁-C₆ alkyl, C₁-C₆ alkenyl, C₁-C₆ alkoxy, C₃-C₆ carbocyclyl, 4- to 6-membered heterocyclyl, —C(═O)R^{c}, —C(═O)OR^{c}, —C(═O)N(R^{c})₂, and —OS(═O)₂R^{c} groups, wherein:
R^{c}, for each occurrence, is independently chosen from hydrogen, C₁-C₄ alkyl, and C₁-C₄ haloalkyl groups;
the 4- to 6-membered heterocyclyl of R¹ comprises 1 to 2 heteroatoms independently chosen from nitrogen and oxygen and is optionally substituted with a group selected from oxo and —OH;
the C₁-C₆ alkyl and C₁-C₆ alkenyl of R¹ are each optionally substituted with 1 to 4 groups independently chosen from halogen, cyano, —OH, —NH₂, —NH(C₁-C₄ alkyl), —N(C₁-C₄ alkyl)₂, C₁-C₄ alkoxy, 3- to 5-membered heterocyclyl (optionally further substituted with R^{c}), 3- to 5-membered heteroaryl groups, and C₆ aryl;
the C₁-C₆ alkoxy of R¹ is optionally substituted with 1 to 3 groups independently chosen from —OH, cyano, and halogen groups;
the C₃-C₆ carbocyclyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups; and
the phenyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups;
R² and R³ are each independently chosen from hydrogen and C₁-C₄ alkyl groups;
R⁴ is chosen from C₁-C₆ alkyl, —C(═O)O(C₁-C₄ alkyl), and 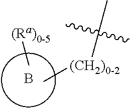
groups, wherein:
the C₁-C₆ alkyl of R⁴ is optionally substituted with 1 to 5 groups independently chosen from halogen, cyano, —OH, —NH₂, —NH(C₁-C₄ alkyl), —N(C₁-C₄ alkyl)₂, —C(═O)NH₂, —C(═O)(C₁-C₄ alkyl), —C(═O)OH, —C(═O)O(C₁-C₄ alkyl), —C(═O)NH(C₁-C₄ alkyl), —C(═O)N(C₁-C₄ alkyl)₂, C₁-C₄ alkoxy, C₃-C₆ carbocyclyl, C₆ aryl, —O—(C₆ aryl), 5- to 10-membered heterocyclyl, and 5- to 10-membered heteroaryl groups, wherein:
the C₆ aryl and —O—(C₆ aryl) groups are each optionally substituted with 1 to 3 groups independently chosen from halogen and C₁-C₄ haloalkyl groups;
Ring B is chosen from C₃-C₁₂ carbocyclyl, 3- to 12-membered heterocyclyl, C₆ and C₁₀ aryl, and 5- to 10-membered heteroaryl groups, wherein Ring B is optionally substituted with 1, 2, 3, 4, or 5 R^{a} groups; wherein:
R^{a}, for each occurrence, is independently chosen from halogen, cyano, oxo, C₁-C₈ alkyl, C₁-C₆ haloalkyl, C₂-C₅ alkenyl, C₁-C₆ haloalkenyl, C₁-C₆ alkoxy, C₁-C₆ haloalkoxy, C₃-C₁₂ carbocyclyl, C₆ and C₁₀ aryl, 3- to 12-membered heterocyclyl, 5- to 10-membered heteroaryl, —C(═O)NR^{h}Rⁱ, —C(═O)OR^{k},
—C(═O)(C₁-C₄ alkylene)OR^{k}, —C(═O)R^{k}, —C(═O)(C₁-C₄ alkylene)S(═O)ₚR^{k}, —C(═O)(C₁-C₄ alkylene)S(═O)ₚNR^{h}Rⁱ, —C(═O)(C₁-C₄ alkylene)NRⁱS(═O)ₚR^{k}, —C(═O)(C₁-C₄ alkylene)NR^{h}C(═O)R^{k}, —C(═O)C(═O)R^{k},
—NR^{h}Rⁱ, —NH(CH₂)_{q}CHR^{h}Rⁱ, —NH(CH₂)_{q}NR^{h}Rⁱ, —NR^{h}C(═O)R^{k}, —NR^{h}C(═O)OR^{k}, —NR^{h}C(═O)(C₁-C₄ alkylene)OR^{k}, —NR^{h}C(═O)O(C₁-C₄ alkylene)R^{k},
—NR^{h}C(═O)NRⁱR^{j}, —NR^{h}C(═O)(C₁-C₄ alkylene)NRⁱS(═O)ₚR^{k}, —NR^{h}S(O)R^{k}, —NR^{h}C(═O)(C₁-C₄ alkylene)S(═O)ₚR^{k}, —NR^{h}S(═O)ₚ(C₁-C₄ alkylene)C(═O)OR^{k}, —NR^{h}C(═O)[O(CH₂)_{q}]ᵣOC(═O)NR^{h}Rⁱ(CH₂)_{q}[O(CH₂)_{q}]ᵣ(C₁-C₆ alkyl) (optionally substituted by 1 to 3 R¹ groups), —NR^{h}C(O)(C₁-C₆ alkylene)[O(CH₂)_{q}]ᵣOC(═O)NR^{h}Rⁱ(CH₂)_{q}[O(CH₂)_{q}]ᵣ(C₁-C₆ alkyl) (optionally substituted by 1 to 3 R^{m} groups), —OR^{k}, —OC(═O)R^{k}, —OC(═O)OR^{k}, —OC(═O)NR^{h}Rⁱ, —[O(CH₂)_{q}]ᵣO(C₁-C₆ alkyl), —S(═O)ₚR^{k}, and —S(═O)ₚNR^{h}Rⁱ groups, wherein:
the C₁-C₄ alkylene in each of —C(═O)(C₁-C₄ alkylene)S(═O)ₚR^{k}, —C(═O)(C₁-C₄ alkylene)OR^{k}, —C(═O)(C₁-C₄ alkylene)S(═O)ₚNR^{h}Rⁱ, —C(═O)(C₁-C₄ alkylene)NRⁱS(═O)ₚR^{k}, —C(═O)(C₁-C₄ alkylene)NR^{h}C(═O)R^{k}, —NR^{h}C(═O)O(C₁-C₄ alkylene)R^{k}, —NR^{h}C(═O)(C₁-C₄ alkylene)S(═O)ₚR^{k}—NR^{h}C(═O)(C₁-C₄ alkylene)OR^{k}, —NR^{h}S(═O)p(C₁-C₄ alkylene)C(═O)OR^{k}, and —NR^{h}C(O)(C₁-C₄ alkylene)NRⁱS(═O)ₚR^{k} of R^{a} is optionally substituted with 1 to 3 —OH groups;
the C₁-C₈ alkyl, the C₁-C₆ haloalkyl, the C₁-C₆ alkoxy, and the C₂-C₅ alkenyl of R^{a} are each optionally substituted with 1 to 3 groups independently chosen from cyano, —C(═O)R^{k}, —C(═O)OR^{k}, —C(═O)NR^{h}Rⁱ, =NOR^{k}, —NR^{h}Rⁱ, —NR^{h}C(═O)R^{k}, —NR^{h}C(═O)OR^{k}, —NR^{h}C(═O)NRⁱR^{j}, —NR^{h}S(═O)ₚR^{k}, —OR^{k}, —[O(CH₂)_{q}]ᵣOH, —OC(═O)R^{k}, —OC(═O)OR^{k}, —OC(═O)NR^{h}Rⁱ, —SR^{k}, —S(═O)R^{k}, —S(═O)ₚNR^{h}Rⁱ, —[O(CH₂)_{q}]ᵣO(C₁-C₄ alkyl), —O—(C₆ aryl or 5- to 8-membered heteroaryl) (optionally substituted with 1 to 3 R^{m} groups), C₃-C₆ carbocyclyl (optionally substituted with 1 to 3 R^{m} groups), C₆ to C₁₀ aryl (optionally substituted with 1 to 3 R^{m} groups), 4- to 10-membered heterocyclyl (optionally substituted with 1 to 3 R^{m} groups), and 5- to 10-membered heteroaryl (optionally substituted with 1 to 3 R^{m} groups) groups;
the C₃-C₁₂ carbocyclyl, the 3- to 12-membered heterocyclyl, the C₆ and C₁₀ aryl, and the 5- to 10-membered heteroaryl of R^{a} are each optionally substituted with 1 to 3 groups independently chosen from halogen, oxo, cyano, C₁-C₆ alkyl (optionally substituted with 1 to 3 R^{m} groups), —C(═O)R^{k}, —C(═O)OR^{k}, —NR^{h}Rⁱ, —OR^{k}, —S(O)ₚR^{k}, —S(═O)ₚNR^{h}Rⁱ, and 5- to 10-membered heterocyclyl groups, wherein:
R^{h}, Rⁱ,and R^{j}, for each occurrence, are each independently chosen from hydrogen, C₁-C₆ alkyl, C₆-C₁₀ aryl, C₃-C₈ carbocyclyl (optionally substituted with 1 to 3 R^{m} groups), 5- to 10-membered heteroaryl (optionally substituted with 1 to 3 R^{m} groups), and 5- to 10-membered heterocyclyl (optionally substituted with 1 to 3 R^{m} groups) groups, wherein:
the C₁-C₆ alkyl of any one of R^{h}, Rⁱ, and R^{j} is optionally substituted with 1 to 3 groups independently chosen from halogen, cyano, —OH, C₁-C₄ alkoxy, —C(═O)NH(C₁-C₄ alkyl), C₃-C₆ carbocyclyl (optionally substituted with 1 to 3 R^{m} groups), 5- to 10-membered heteroaryl (optionally substituted with 1 to 3 R^{m} groups), and 5- to 10-membered heterocyclyl (optionally substituted with 1 to 3 R^{m} groups) groups;
R¹, for each occurrence, is independently chosen from hydrogen, C₁-C₆ alkyl, benzyl, C₆ aryl, C₃-C₆ carbocyclyl, 5-to 10-membered heterocyclyl, and 5- to 10-membered heteroaryl groups, wherein:
the C₁-C₆ alkyl of any one of R^{k} is optionally substituted with 1 to 5 groups independently chosen from halogen, cyano, —NH₂, —OH, C₁-C₄ alkoxy, C₃-C₆ cycloalkyl (optionally substituted with 1 to 3 halogen groups), 5- to 10-membered heterocyclyl (optionally substituted with 1 to 3 —OH groups), and 5- to 10-membered heteroaryl (optionally substituted with 1 to 3 —OH groups) groups; and
the C₃-C₆ carbocyclyl, benzyl, and C₆ aryl of any one of R^{k} are each optionally substituted with 1 to 3 groups independently chosen from halogen, cyano, oxo, —OH, —C(═O)NH₂, —C(═O)N(CH₃)₂, C₁-C₆ alkyl, C₁-C₆ haloalkyl, C₁-C₆ alkoxy, C₃-C₆ cycloalkyl (optionally substituted with 1 to 3 halogen groups), C₆ aryl (optionally substituted with 1 to 3 halogen groups), and 5- to 10-membered heteroaryl (optionally substituted with 1 to 3 halogen groups) groups, wherein:
the C₁-C₄ alkyl is optionally substituted by 1 to 3 —OH groups; and
the 5- to 10-membered heteroaryl and 5- to 10-membered heterocyclyl of any one of R^{k} are each optionally substituted with 1 to 3 groups independently chosen from halogen, cyano, —C(═O)CH₃, —NH₂, —OH, C₁-C₄ alkyl, C₁-C₄ haloalkyl, C₃-C₆ cycloalkyl, and C₁-C₄ alkoxy groups, wherein:
the C₁-C₄ alkyl is optionally substituted by 1 to 3 —OH groups;
R^{m}, for each occurrence, is independently chosen from halogen, cyano, oxo, —(CH₂)ₙC(═O)NH₂, —NH₂, —NH(C₁-C₄ alkyl), —N(C₁-C₄ alkyl)₂, C₁-C₆ alkyl, C₁-C₆ alkoxy, —C(═O)R^{k}, —S(═O)ₚR^{k}, —OR^{k}, C₃-C₆ cycloalkyl, and 5- to 10-membered heterocyclyl groups, wherein:
the C₁-C₆ alkyl, the C₁-C₆ alkoxy, and the 5- to 10-membered heterocyclyl of any one of R^{m} is optionally substituted with 1 to 3 groups independently chosen from halogen, cyano, —OH, and C₁-C₄ alkoxy groups; and
n, for each occurrence, is an integer chosen from 0, 1, and 2;
p, for each occurrence, is an integer independently chosen from 1 and 2; and
q and r, for each occurrence, are integers independently chosen from 0, 1, 2, and 3.

2. The compound, tautomer, deuterated derivative, or pharmaceutically acceptable salt according to claim 1, wherein:
Ring A is phenyl optionally substituted by 1, 2, or 3 R¹ groups;
R^{1a}, R^{1b}, and R^{1c}, for each occurrence, are each independently chosen from hydrogen and R¹ groups;
R¹, for each occurrence, is independently chosen from halogen, —OH, cyano, C₁-C₆ alkyl, C₁-C₆ alkoxy, 4- to 6-membered heterocyclyl, —C(═O)OR^{c}, —C(═O)R^{c}, and —C(═O)N(R^{c})₂ groups, wherein:
R^{c}, for each occurrence, is independently chosen from hydrogen and C₁-C₄ alkyl groups;
the 4- to 6-membered heterocyclyl of R¹ comprises one heteroatom chosen from nitrogen and oxygen;
the C₁-C₆ alkyl of R¹ is optionally substituted with 1 to 4 groups independently chosen from halogen, cyano, —OH, C₁-C₄ alkoxy, 3- to 5-membered heterocyclyl, and 3-to 5-membered heteroaryl groups; and
the C₁-C₆ alkoxy of R¹ is optionally substituted with 1 to 3 independently chosen halogen atoms;
R² and R³ are each independently chosen from hydrogen and C₁-C₄ alkyl groups;
R⁴ is chosen from C₁-C₆ alkyl and 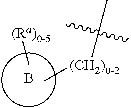
groups, wherein:
the C₁-C₆ alkyl of R⁴ is optionally substituted with 1 to 3 groups independently chosen from halogen, cyano, —OH, C₆ aryl, 5- to 10-membered heterocyclyl, and 5- to 10-membered heteroaryl groups, wherein:
the C₆ aryl is optionally substituted with 1 to 3 independently chosen halogen atoms;
Ring B is chosen from C₃-C₁₂ carbocyclyl, 3- to 12-membered heterocyclyl, C₆ aryl, and 5- to 10-membered heteroaryl groups, wherein Ring B is optionally substituted with 1, 2, or 3 R^{a} groups; wherein:
R^{a}, for each occurrence, is independently chosen from halogen, cyano, C₁-C₈ alkyl, C₁-C₆ haloalkyl, C₂-C₅ alkenyl, C₁-C₆ haloalkenyl, C₁-C₆ alkoxy, C₁-C₆ haloalkoxy, C₃-C₁₂ carbocyclyl, C₆ and C₁₀ aryl, 3- to 12-membered heterocyclyl, 5- to 10-membered heteroaryl, —C(═O)NR^{h}Rⁱ, —C(═O)OR^{k}, —NR^{h}Rⁱ,
—NR^{h}C(O)R^{k}, —NR^{h}C(═O)OR^{k}, —NR^{h}S(O)ₚR^{k}, —S(═O)ₚR^{k}, and —S(═O)ₚNR^{h}Rⁱ groups, wherein:
the C₁-C₈ alkyl, the C₁-C₆ haloalkyl, the C₁-C₆ alkoxy, and the C₂-C₅ alkenyl of R^{a} are each optionally substituted with 1 to 3 groups independently chosen from —C(═O)R^{k}, —C(═O)OR^{k}, —C(═O)NR^{h}Rⁱ, —NR^{h}Rⁱ, —NR^{h}C(═O)R^{k}, —NR^{h}C(═O)OR^{k}, —NR^{h}C(═O)NRⁱR^{j}, —NR^{h}S(═O)ₚR^{k}, —OR^{k}, —OC(═O)R^{k}, —OC(═O)OR^{k}, —OC(═O)NR^{h}Rⁱ, —SR^{k}, —S(O)ₚR^{k}, —S(═O)ₚNR^{h}Rⁱ, C₃-C₆ carbocyclyl (optionally substituted with 1 to 3 R^{m} groups), C₆ to C₁₀ aryl (optionally substituted with 1 to 3 R^{m} groups), 4- to 10-membered heterocyclyl (optionally substituted with 1 to 3 R^{m} groups), and 5- to 10-membered heteroaryl (optionally substituted with 1 to 3 R^{m} groups) groups;
the C₃-C₁₂ carbocyclyl, the 3- to 12-membered heterocyclyl, the C₆ and C₁₀ aryl, and the 5- to 10-membered heteroaryl of R^{a} are each optionally substituted with 1 to 3 groups independently chosen from halogen, C₁-C₆ alkyl (optionally substituted with 1 to 3 R^{m} groups),
—NR^{h}Rⁱ, —S(═O)ₚR^{k}, and —S(═O)ₚNR^{h}Rⁱ groups, wherein:
R^{h}, Rⁱ,and R^{j}, for each occurrence, are each independently chosen from hydrogen, C₁-C₆ alkyl, 5- to 10-membered heteroaryl (optionally substituted with 1 to 3 R^{m} groups), and 5- to 10-membered heterocyclyl (optionally substituted with 1 to 3 R^{m} groups) groups, wherein:
the C₁-C₆ alkyl of any one of R^{h}, Rⁱ, and R^{j} is optionally substituted with 1 to 3 groups independently chosen from halogen and —OH groups;
R^{k}, for each occurrence, is independently chosen from hydrogen, C₁-C₆ alkyl, benzyl, C₆ aryl, C₃-C₆ carbocyclyl, 5-to 10-membered heterocyclyl, and 5- to 10-membered heteroaryl groups, wherein:
the C₁-C₆ alkyl of any one of R^{k} is optionally substituted with 1 to 5 groups independently chosen from halogen, cyano, —NH₂, —OH, C₁-C₄ alkoxy, C₃-C₆ cycloalkyl (optionally substituted with 1 to 3 halogen groups), 5- to 10-membered heterocyclyl (optionally substituted with 1 to 3 —OH groups), and 5- to 10-membered heteroaryl (optionally substituted with 1 to 3 —OH groups) groups; and
the 5- to 10-membered heteroaryl and 5- to 10-membered heterocyclyl of any one of R^{k} are each optionally substituted with 1 to 3 groups independently chosen from halogen, C₁-C₄ alkyl, C₁-C₄ haloalkyl, C₃-C₆ cycloalkyl, and C₁-C₄ alkoxy groups;
R^{m}, for each occurrence, is independently chosen from oxo, halogen, cyano, —(CH₂)ₙC(═O)NH₂, —NH₂, —NH(C₁-C₄ alkyl), —N(C₁-C₄ alkyl)₂, C₁-C₆ alkyl, —OR^{k}, and C₃-C₆ cycloalkyl groups, wherein:
the C₁-C₆ alkyl of any one of R^{m} is optionally substituted with 1 to 3 groups independently chosen from halogen and —OH groups; and
n, for each occurrence, is an integer chosen from 0, 1, and 2;
p, for each occurrence, is an integer independently chosen from 1 and 2; and
all other variables not specifically defined herein are as defined in claim 1.

3. The compound, tautomer, deuterated derivative, or pharmaceutically acceptable salt according to claim 1 or 2, wherein:
Ring A is phenyl, optionally substituted by 1, 2, or 3 R¹ groups;
R^{1a}, R^{1b}, and R^{1c}, for each occurrence, are each independently chosen from hydrogen and R¹ groups;
R¹, for each occurrence, is independently chosen from halogen, —OH, cyano, C₁-C₆ alkyl, C₁-C₆ alkoxy, 4- to 6-membered heterocyclyl, —C(═O)OR^{c}, —C(═O)R^{c}, and —C(═O)N(R^{c})₂ groups, wherein:
R^{c}, for each occurrence, is independently chosen from hydrogen and C₁-C₄ alkyl groups;
the 4- to 6-membered heterocyclyl of R¹ comprises one heteroatom chosen from nitrogen and oxygen;
the C₁-C₆ alkyl of R¹ is optionally substituted with 1 to 4 groups independently chosen from halogen, —OH, C₁-C₄ alkoxy, 3- to 5-membered heterocyclyl, and 3- to 5-membered heteroaryl groups; and
the C₁-C₆ alkoxy of R¹ is optionally substituted with 1 to 3 independently chosen halogen atoms;
R² and R³ are each independently chosen from hydrogen and C₁-C₂ alkyl groups;
R⁴ is chosen from C₁-C₆ alkyl and 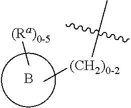
groups, wherein:
the C₁-C₆ alkyl of R⁴ is optionally substituted with 1 to 3 groups independently chosen from —OH and C₆ aryl groups, wherein:
the C₆ aryl is optionally substituted with 1 to 3 independently chosen halogen atoms;
Ring B is chosen from C₃-C₁₂ carbocyclyl, 3- to 12-membered heterocyclyl, C₆ aryl, and 5- to 10-membered heteroaryl groups, wherein Ring B is optionally substituted with 1, 2, or 3 R^{a} groups; wherein:
R^{a}, for each occurrence, is independently chosen from halogen, cyano, C₁-C₈ alkyl, C₁-C₆ haloalkyl, C₂-C₅ alkenyl, C₁-C₆ alkoxy, C₁-C₆ haloalkoxy, C₃-C₁₂ carbocyclyl, C₆ aryl, 3- to 12-membered heterocyclyl, 5- to 10-membered heteroaryl, —C(═O)NR^{h}Rⁱ, —C(═O)OR^{k}, —NR^{h}Rⁱ, —NR^{h}C(═O)R^{k}, —NR^{h}C(═O)OR^{k}, —NR^{h}S(═O)ₚR^{k}, —S(═O)ₚR^{k}, and —S(═O)ₚNR^{h}Rⁱ groups, wherein:
the C₁-C₈ alkyl, the C₁-C₆ haloalkyl, the C₁-C₆ alkoxy, and the C₂-C₅ alkenyl of R^{a} are each optionally substituted with 1 to 3 groups independently chosen from —C(═O)NR^{h}Rⁱ, —NR^{h}Rⁱ, —NR^{h}C(═O)NRⁱR^{j}, —NR^{h}S(═O)ₚR^{k}, —OR^{k}, —S(═O)ₚR^{k}, 5- to 10-membered heterocyclyl (optionally substituted with 1 to 3 R^{m} groups), and 5- to 10-membered heteroaryl (optionally substituted with 1 to 3 R^{m} groups) groups;
the C₃-C₁₂ carbocyclyl, the 3- to 12-membered heterocyclyl, the C₆ aryl, and the 5- to 10-membered heteroaryl of R^{a} are each optionally substituted with 1 to 3 groups independently chosen from C₁-C₆ alkyl (optionally substituted with 1 to 3 R^{m} groups) and —S(═O)ₚR^{k} groups, wherein:
R^{h}, Rⁱ, and R^{j}, for each occurrence, are each independently chosen from hydrogen, C₁-C₆ alkyl, and 5- to 10-membered heteroaryl (optionally substituted with 1 to 3 R^{m} groups) groups, wherein:
the C₁-C₆ alkyl of any one of R^{h}, Rⁱ, and R^{j} is optionally substituted with 1 to 3 —OH groups;
R^{k}, for each occurrence, is independently chosen from hydrogen, C₁-C₆ alkyl, C₃-C₆ carbocyclyl, 5- to 10-membered heterocyclyl, and 5- to 10-membered heteroaryl groups, wherein:
the C₁-C₆ alkyl of any one of R^{k} is optionally substituted with a cyano group; and
the 5- to 10-membered heteroaryl and 5- to 10-membered heterocyclyl of any one of R^{k} are each optionally substituted with 1 to 3 groups independently chosen from halogen, C₁-C₄ alkyl, and C₃-C₆ cycloalkyl groups;
R^{m}, for each occurrence, is independently chosen from halogen, cyano, —(CH₂)ₙC(═O)NH₂, —NH(C₁-C₄ alkyl), —N(C₁-C₄ alkyl)₂, C₁-C₆ alkyl, —OR^{k}, and C₃-C₆ cycloalkyl groups, wherein:
the C₁-C₆ alkyl of any one of R^{m} is optionally substituted with 1 to 3 —OH groups; and
n, for each occurrence, is an integer chosen from 0 and 1;
p, for each occurrence, is 2; and
all other variables not specifically defined herein are as defined in claim 1.

4. The compound, tautomer, deuterated derivative, or pharmaceutically acceptable salt according to any one of claims 1 to 3, wherein:
R⁴ is chosen from 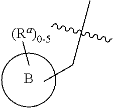
groups, wherein Ring B is optionally substituted with 1, 2, or 3 R^{a} groups; and
all other variables not specifically defined herein are as defined in any one of claims 1 to 3.

5. The compound, tautomer, deuterated derivative, or pharmaceutically acceptable salt according to any one of claims 1 to 4, wherein:
X is chosen from —CR^{1a}R^{1b}—, —C(O)—, and —O—;
Y is chosen from —CR^{1a}R^{1b}— and —NR^{1c}—;
Z is chosen from a bond and —CR^{1a}R^{1b}—, wherein:
at least one of X and Y is chosen from —CR^{1a}R^{1b}— and —C(O)—; and
all other variables not specifically defined herein are as defined in any one of claims 1 to 4.

6. The compound, tautomer, deuterated derivative, or pharmaceutically acceptable salt according to any one of claims 1 to 5, wherein the compound is represented by the following structural formula: 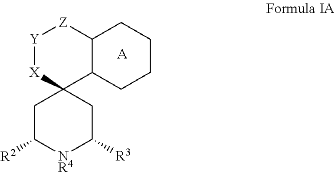
a tautomer thereof, a deuterated derivative of that compound or tautomer, or a pharmaceutically acceptable salt of any of the foregoing, wherein all variables not specifically defined herein are as defined in any one of claims 1 to 5.

7. The compound, tautomer, deuterated derivative, or pharmaceutically acceptable salt according to any one of claims 1 to 5, wherein the compound is represented by one of the following structural formulae: 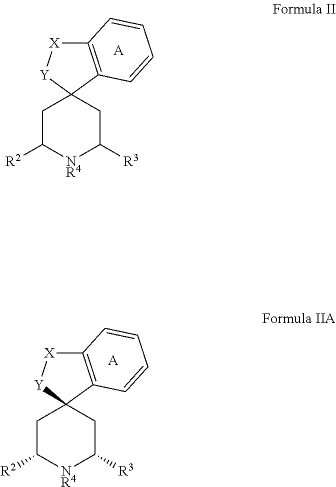
a tautomer thereof, a deuterated derivative of that compound or tautomer, or a pharmaceutically acceptable salt of any of the foregoing, wherein:
Ring A is optionally substituted by 1 or 2 R¹ groups; and
all variables not specifically defined herein are as defined in any one of claims 1 to 5.

8. The compound, tautomer, deuterated derivative, or pharmaceutically acceptable salt according to claims 1 to 5, wherein the compound is represented by the following structural formula: 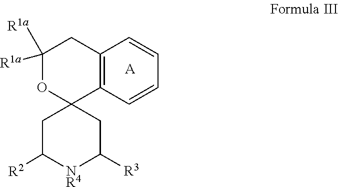
a tautomer thereof, a deuterated derivative of that compound or tautomer, or a pharmaceutically acceptable salt of any of the foregoing, wherein:
Ring A is optionally substituted by 1 or 2 R¹ groups; and
all variables not specifically defined herein are as defined in any one of claims 1 to 5.

9. The compound, tautomer, deuterated derivative, or pharmaceutically acceptable salt according to claims 1 to 5, wherein the compound is represented by one of the following structural formulae: 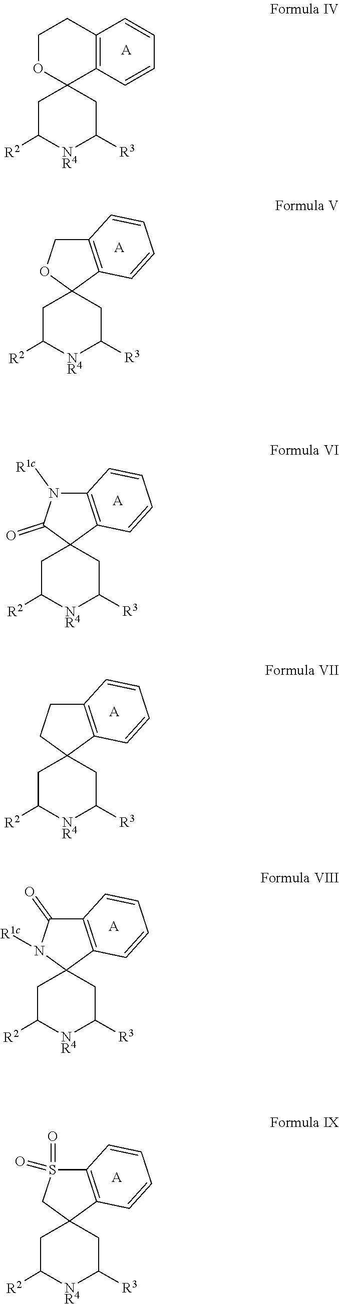
a tautomer thereof, a deuterated derivative of that compound or tautomer, or a pharmaceutically acceptable salt of any of the foregoing, wherein:
Ring A is optionally substituted by 1 or 2 R¹ groups; and
all variables not specifically defined herein are as defined in any one of claims 1 to 5.

10. The compound, tautomer, deuterated derivative, or pharmaceutically acceptable salt according to claims 1 to 5, wherein the compound is represented by one of the following structural formulae: 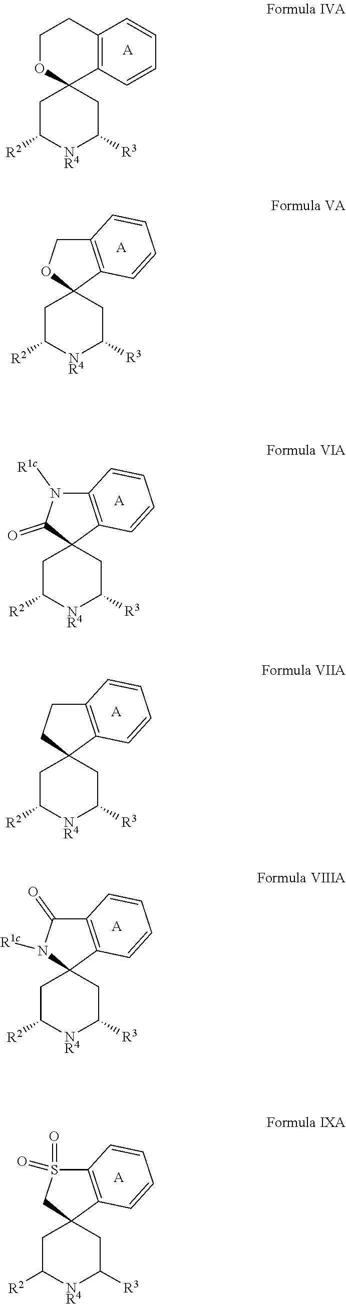
a tautomer thereof, a deuterated derivative of that compound or tautomer, or a pharmaceutically acceptable salt of any of the foregoing, wherein:
Ring A is optionally substituted by 1 or 2 R¹ groups; and
all variables not specifically defined herein are as defined in any one of claims 1 to 5.

11. The compound, tautomer, deuterated derivative, or pharmaceutically acceptable salt according to any one of claims 1 to 5, wherein the compound is represented by one of the following structural formulae: 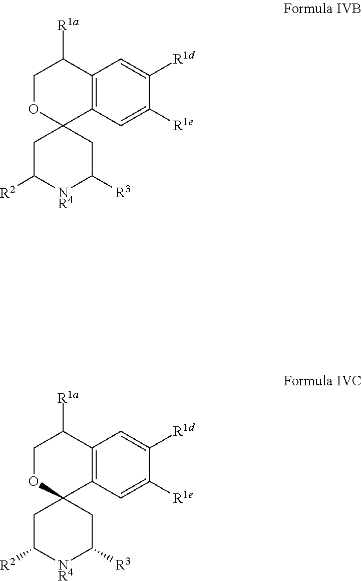
a tautomer thereof, a deuterated derivative of that compound or tautomer, or a pharmaceutically acceptable salt of any of the foregoing, wherein:
R^{1a} is chosen from hydrogen, halogen, —OH, and phenyl groups, wherein:
the phenyl of R^{1a} is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy,
—C(═O)NH₂, —C(═O)NH(C₁-C₄ alkyl), and —C(═O)N(C₁-C₄ alkyl)₂ groups;
R^{1d} and R^{1e} are each independently chosen from hydrogen, halogen, —OH, cyano,
C₁-C₄ alkyl, C₁-C₄ alkoxy, —C(═O)OR^{c}, —C(═O)N(R^{c})₂, and —OS(═O)₂R^{c} groups, wherein:
R^{c}, for each occurrence, is independently chosen from hydrogen, C₁-C₄ alkyl, and C₁-C₄ haloalkyl groups; and
the C₁-C₆ alkyl of R^{1d} and/or Re is optionally substituted with 1 to 3 groups independently chosen from halogen and —OH groups; and
all variables not specifically defined herein are as defined in any one of claims 1 to 5.

12. The compound, tautomer, deuterated derivative, or pharmaceutically acceptable salt according to any one of claims 1 to 5, wherein the compound is represented by one of the following structural formulae: 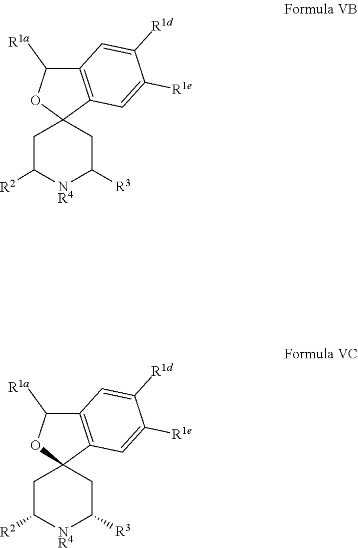
a tautomer thereof, a deuterated derivative of that compound or tautomer, or a pharmaceutically acceptable salt of any of the foregoing, wherein:
R^{1a} is chosen from hydrogen, phenyl, and C(═O)N(R^{c1})₂ groups, wherein:
the phenyl of R^{1a} is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups;
R^{c1}, for each occurrence, is independently chosen from hydrogen and C₁-C₄ alkyl groups;
R^{1d} and R^{1e} are each independently chosen from hydrogen and halogen groups; and
all variables not specifically defined herein are as defined in any one of claims 1 to 5.

13. The compound, tautomer, deuterated derivative, or pharmaceutically acceptable salt according to any one of claims 1 to 5, wherein the compound is represented by one of the following structural formulae: 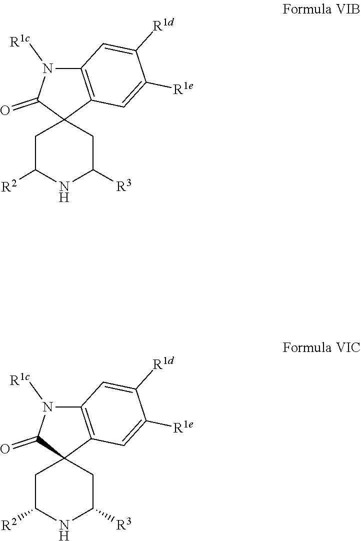
a tautomer thereof, a deuterated derivative of that compound or tautomer, or a pharmaceutically acceptable salt of any of the foregoing, wherein:
R^{1d} and R^{1e} are each independently chosen from hydrogen, halogen, C₁-C₄ alkyl, and C₁-C₄ haloalkyl groups; and
all variables not specifically defined herein are as defined in any one of claims 1 to 5.

14. The compound, tautomer, deuterated derivative, or pharmaceutically acceptable salt according to claim 1, wherein Ring A is an unsubstituted phenyl group.

15. The compound, tautomer, deuterated derivative, or pharmaceutically acceptable salt according to claim 1, wherein Ring A is phenyl substituted with 1 or 2 R¹ groups.

16. The compound, tautomer, deuterated derivative, or pharmaceutically acceptable salt according to claim 15, wherein each R¹ is independently chosen from halogen, cyano, —CH₃, —CH₂CH₃, —CH₂CF₂, —CFCF₂, —CF₂, —CF₃, —OCH₃, —OCF₂, and —OCF₃.

17. The compound, tautomer, deuterated derivative, or pharmaceutically acceptable salt according to claim 15, wherein each R¹ is independently chosen from Cl and F.

18. The compound, tautomer, deuterated derivative, or pharmaceutically acceptable salt according to any one of claims 1 and 14 to 17, wherein X is chosen from —C(O)—, —O—, —S—, —CR^{1a}R^{1b}—, and —NR^{1c}—, wherein R^{1a}, R^{1b}, and R^{1c} are as defined in claim 1.

19. The compound, tautomer, deuterated derivative, or pharmaceutically acceptable salt according to claim 18, wherein X is chosen from —CH₂— and —NH—.

20. The compound, tautomer, deuterated derivative, or pharmaceutically acceptable salt according to claim 18, wherein X is —CR^{1a}R^{1b}—, wherein R^{1a} is hydrogen and R^{1b} is chosen from R¹ groups.

20. The compound, tautomer, deuterated derivative, or pharmaceutically acceptable salt according to claim 18, wherein X is —CHOH—.

21. The compound, tautomer, deuterated derivative, or pharmaceutically acceptable salt according to claim 18, wherein X is —O—.

22. The compound, tautomer, deuterated derivative, or pharmaceutically acceptable salt according to claim 18, wherein X is —C(O)—.

23. The compound, tautomer, deuterated derivative, or pharmaceutically acceptable salt according to any one of claims 1, and 14 to 22, wherein Y is chosen from —CR^{1a}R^{1b}— and —NR^{1c}—.

24. The compound, tautomer, deuterated derivative, or pharmaceutically acceptable salt according to claim 23, wherein Y is —CH₂.

25. The compound, tautomer, deuterated derivative, or pharmaceutically acceptable salt according to claim 23, wherein Y is —CR^{1a}R^{1b}, wherein R^{1a} and R^{1b} are each R¹, and R¹ is —CH₃.

26. The compound, tautomer, deuterated derivative, or pharmaceutically acceptable salt according to claim 23, wherein Y is —CR^{1a}R^{1b}, R^{1a} is hydrogen, and R^{1b} is chosen from R¹ groups as defined in claim 1.

27. The compound, tautomer, deuterated derivative, or pharmaceutically acceptable salt according to claim 23, wherein Y is —CR^{1a}R^{1b}, R^{1a} is hydrogen, R^{1b} is R¹, and R¹ is chosen from —OH, —CH₃, —C(O)NH₂, C(O)NHCH₃.

28. The compound, tautomer, deuterated derivative, or pharmaceutically acceptable salt according to claim 23, wherein Y is —CR^{1a}R^{1b}, R^{1a} and R^{1b} are both R¹, and R¹ is chosen from
—OH and —CH3.

29. The compound, tautomer, deuterated derivative, or pharmaceutically acceptable salt according to claim 23, wherein Y is —NR^{1c}—, wherein R^{1c} is R¹, and R¹ is chosen from 4- to 6-membered heterocyclyl.

30. The compound, tautomer, deuterated derivative, or pharmaceutically acceptable salt according to claim 23, wherein Y is is —NR^{1c}—, wherein R^{1c} is R¹, and R¹ is

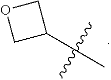 [SMILES: *C1COC1]

31. The compound, tautomer, deuterated derivative, or pharmaceutically acceptable salt according to claim 23, wherein Y is —N(CH₃)—.

32. The compound, tautomer, deuterated derivative, or pharmaceutically acceptable salt according to claim 23, wherein Y is —CH(OH)—.

33. The compound, tautomer, deuterated derivative, or pharmaceutically acceptable salt according to claim 23, wherein Y is —NH—.

33. The compound, tautomer, deuterated derivative, or pharmaceutically acceptable salt according to claim 23, wherein Y is —N(C(O)CH₃)—.

34. The compound, tautomer, deuterated derivative, or pharmaceutically acceptable salt according to claim 1, wherein X is chosen from —C(O)— and —CR^{1a}R^{1b}—, and Y is —NR^{1c}—, wherein R^{1c} is hydrogen.

35. The compound, tautomer, deuterated derivative, or pharmaceutically acceptable salt according to claim 1, wherein X is chosen from —C(O)— and —CR^{1a}R^{1b}—, and Y is —NR^{1c}—, wherein R^{1c} is R¹, and R¹ is chosen from C₁-C₆ alkyl and C₁-C₆ alkoxy optionally substituted with 1 to 3 halogen groups.

36. The compound, tautomer, deuterated derivative, or pharmaceutically acceptable salt according to claim 1, wherein X is chosen from —C(O)— and —CR^{1a}R^{1b}—, and Y is —NR^{1c}—, wherein R^{1c} is R¹, and R^{E} is chosen from —CH₃, —CH₂CH₃, —CH₂CH₂OH, —CH₂CF₂, —CH₂CF₃, and —CH₂CH₂OCH₃.

37. The compound, tautomer, deuterated derivative, or pharmaceutically acceptable salt according to any one of claims 4-36, Z is chosen from a bond, —CR^{1a}R^{1b}—, —NR^{1c}—, —C(O)—, —S(O)₂—, and —O—.

38. The compound, tautomer, deuterated derivative, or pharmaceutically acceptable salt according to claim 37, wherein Z is a bond.

39. The compound, tautomer, deuterated derivative, or pharmaceutically acceptable salt according to claim 37, wherein Z is chosen from —CR^{1a}R^{1b}—.

40. The compound, tautomer, deuterated derivative, or pharmaceutically acceptable salt according to claim 39, wherein R^{1a} and R^{1b} are hydrogen.

41. The compound, tautomer, deuterated derivative, or pharmaceutically acceptable salt according to claim 39, wherein R^{1a} and R^{1b} are fluorine.

42. The compound, tautomer, deuterated derivative, or pharmaceutically acceptable salt according to claim 39, wherein R^{1a} is hydrogen and R^{1b} is —OH.

43. The compound, tautomer, deuterated derivative, or pharmaceutically acceptable salt according to claim 39, wherein R² and R³ are independently chosen from hydrogen and CH₃.

44. The compound, tautomer, deuterated derivative, or pharmaceutically acceptable salt according to claim 39, wherein one of R² and R³ is hydrogen and the other is CH₃.

45. The compound, tautomer, deuterated derivative, or pharmaceutically acceptable salt according to any one of claims 1 to 44, wherein R⁴ is chosen from C₁-C₆ alkyl, optionally substituted with 1 to 5 groups independently chosen from halogen, cyano, —OH, —NH₂, —NH(C₁-C₄ alkyl), —N(C₁-C₄ alkyl)₂, —C(═O)NH₂, —C(═O)(C₁-C₄ alkyl), —C(═O)OH, —C(═O)O(C₁-C₄ alkyl), —C(═O)NH(C₁-C₄ alkyl), —C(═O)N(C₁-C₄ alkyl)₂, C₁-C₄ alkoxy, C₃-C₆ carbocyclyl, C₆ aryl (optionally substituted with 1 to 3 groups independently chosen from halogen and C₁-C₄ haloalkyl groups), —O—(C₆ aryl) (optionally substituted with 1 to 3 groups independently chosen from halogen and C₁-C₄ haloalkyl groups), 5- to 10-membered heterocyclyl, and 5- to 10-membered heteroaryl groups.

46. The compound, tautomer, deuterated derivative, or pharmaceutically acceptable salt according to claim 45, wherein R⁴ is chosen from C₁-C₆ alkyl substituted with 1 to 2 groups independently chosen from —OH, phenyl, and phenyl further substituted with halogen.

47. The compound, tautomer, deuterated derivative, or pharmaceutically acceptable salt according to claim 45, wherein R⁴ is chosen from C₁-C₆ alkyl substituted with —OH and phenyl substituted with C₁.

48. The compound, tautomer, deuterated derivative, or pharmaceutically acceptable salt according to claim 45, wherein R⁴ is chosen from C₁-C₆ alkyl substituted with —OH and phenyl substituted with 2F.

49. The compound, tautomer, deuterated derivative, or pharmaceutically acceptable salt according to claim 45, wherein R⁴ is chosen from —C(═O)O(C₁-C₄ alkyl).

50. The compound, tautomer, deuterated derivative, or pharmaceutically acceptable salt according to any one of claims 1 to 44, wherein R⁴ is chosen from 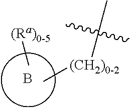
groups.

51. The compound, tautomer, deuterated derivative, or pharmaceutically acceptable salt according to claim 50, wherein R⁴ is chosen from 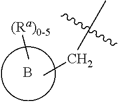
groups.

52. The compound, tautomer, deuterated derivative, or pharmaceutically acceptable salt according to claim 50 or claim 51, wherein Ring B is chosen from

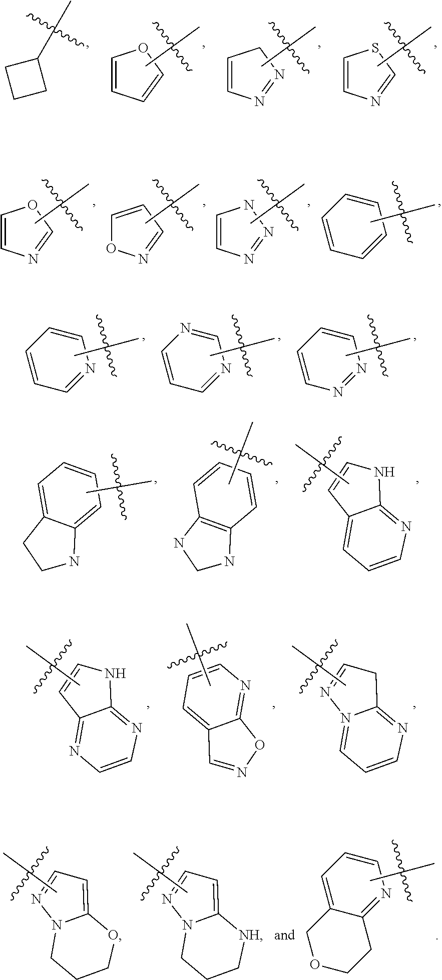

53. The compound, tautomer, deuterated derivative, or pharmaceutically acceptable salt according to any one of claims 1-44 and 50-52, wherein Ring B is unsubstituted.

54. The compound, tautomer, deuterated derivative, or pharmaceutically acceptable salt according to any one of claims 1-44 and 50-52, wherein Ring B is substituted with 1, 2, or 3 R^{a} groups.

55. The compound, tautomer, deuterated derivative, or pharmaceutically acceptable salt according to any one of claims 1-44 and 50-52, wherein each R^{a} group is independently selected from halogen, cyano, oxo, C₁-C₈ alkyl, C₁-C₆ haloalkyl, C₁-C₆ alkoxy, C₁-C₆ haloalkoxy, C₃-C₁₂ carbocyclyl, C₆ and C₁₀ aryl, 3- to 12-membered heterocyclyl, 5- to 10-membered heteroaryl, —C(═O)NR^{h}Rⁱ, —C(═O)OR^{k}, —NR^{h}Rⁱ, —NR^{h}C(═O)R^{k}, —NR^{h}C(O)OR^{k}, —NR^{h}S(═O)ₚR^{k}, —S(═O)ₚR^{k}, and —S(═O)ₚNR^{h}Rⁱ groups, wherein:
the C₁-C₈ alkyl, the C₁-C₆ haloalkyl, the C₁-C₆ alkoxy, and the C₂-C₅ alkenyl of R^{a} are each optionally substituted with 1 to 3 groups independently chosen from —C(═O)NR^{h}Rⁱ, =NOR^{k}, —NR^{h}Rⁱ, —NR^{h}C(═O)NRⁱR^{j}, —NR^{h}S(═O)ₚR^{k}, —OR^{k}, S(═O)ₚR^{k}, —S(═O)ₚNR^{h}Rⁱ, C₃-C₆ carbocyclyl (optionally substituted with 1 to 3 R^{m} groups), 4- to 10-membered heterocyclyl (optionally substituted with 1 to 3 R^{m} groups), and 5- to 10-membered heteroaryl (optionally substituted with 1 to 3 R^{m} groups) groups;
the C₃-C₁₂ carbocyclyl, the 3- to 12-membered heterocyclyl, the C₆ and C₁₀ aryl, and the 5- to 10-membered heteroaryl of R^{a} are each optionally substituted with 1 to 3 groups independently chosen from oxo, halogen, cyano, C₁-C₆ alkyl (optionally substituted with 1 to 3 R^{m} groups), —OR^{k}, —S(═O)ₚR^{k}, —S(═O)ₚNR^{h}Rⁱ, and 5- to 10-membered heterocyclyl groups, wherein:
R^{h}, Rⁱ, and R^{j}, for each occurrence, are each independently chosen from hydrogen, C₁-C₆ alkyl, 5- to 10-membered heteroaryl (optionally substituted with 1 to 3 R^{m} groups), and 5- to 10-membered heterocyclyl (optionally substituted with 1 to 3 R^{m} groups) groups, wherein:
the C₁-C₆ alkyl of any one of Rh, R¹, and Rⁱ is optionally substituted with 1 to 3 groups independently chosen from halogen, cyano, —OH, C₃-C₆ carbocyclyl (optionally substituted with 1 to 3 R^{m} groups);
R^{k}, for each occurrence, is independently chosen from hydrogen, C₁-C₆ alkyl, C₃-C₆ carbocyclyl, and 5- to 10-membered heteroaryl groups, wherein:
the C₁-C₆ alkyl of any one of R^{k} is optionally substituted with 1 to 5 groups independently chosen from halogen, cyano, —NH₂, and —OH; and
the 5- to 10-membered heteroaryl and 5- to 10-membered heterocyclyl of any one of R^{k}are each optionally substituted with 1 to 3 groups independently chosen from halogen, cyano, —OH, C₁-C₄ alkyl (optionally substituted by 1 to 3 —OH groups) C₁-C₄ haloalkyl, C₃-C₆ cycloalkyl, and C₁-C₄ alkoxy groups, wherein:
R^{m}, for each occurrence, is independently chosen from halogen, cyano, oxo, —C(═O)NH₂, —NH₂, —C₁-C₆ alkyl, C₁-C₆ alkoxy, —OR^{k}, and C₃-C₆ cycloalkyl, wherein:
the C₁-C₆ alkyl, the C₁-C₆ alkoxy, and the 5- to 10-membered heterocyclyl of any one of R^{m} is optionally substituted with 1 to 3 groups independently chosen from halogen, cyano, —OH, and C₁-C₄ alkoxy groups;
p, for each occurrence, is an integer independently chosen from 1 and 2.

56. The compound, tautomer, deuterated derivative, or pharmaceutically acceptable salt according to any one of claims 1-44, 50-52, and 54-55, wherein each R^{a} group is independently selected from
-halogen;
-oxo;
-cyano;
—C₁ haloalkoxy;
—CH₃;
—CF₂;
—C₁ alkyl substituted with —OR^{k}, wherein R^{k} is —CH₃;
—C₁ alkyl substituted with —C(═O)NR^{h}Rⁱ, wherein R^{h} and Rⁱ are both CH₃;
—C₁ alkyl substituted with —C(═O)NR^{h}Rⁱ, wherein R^{h} is hydrogen and Rⁱ is CH₃;
—C₁ alkyl substituted with —C(═O)NR^{h}Rⁱ, wherein R^{h} is hydrogen and Rⁱ is CH₃ substituted with C₃-C₆ carbocyclyl optionally substituted cyano;
—C₁ alkyl substituted with —C(═O)NR^{h}Rⁱ, wherein R^{h} is hydrogen and Rⁱ is CH₃ substituted with a 5-membered heteroaryl;
—C₁ alkyl substituted with a cyclopropyl, which is further substituted with R^{m}, wherein R^{m} is selected from CH₃, CH₂OH, and CH₂C(═O)NH₂;
—C₁ alkyl substituted with a 4-membered heterocycle, 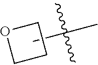
which is further substituted with R^{m}, wherein R^{m} is C(═O)NH₂;
—C₁ alkyl substituted with a 5-membered optionally substituted heterocycle;
—C₁ alkyl substituted with 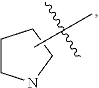
which is optionally substituted with oxo and CH₃;
—C₁ alkyl substituted with a 5-membered optionally substituted heteroaryl;
—C₁ alkyl substituted with 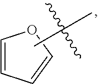
which is optionally substituted with —OH or C(═O)NH₂;
—C₁ alkyl substituted with 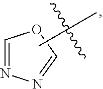
which is optionally substituted with —C₁ to —C₃ alkyl;
—C₁ alkyl substituted with 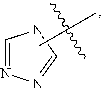
which is optionally substituted with N(CH₃)₂;
—C₁ alkyl substituted with 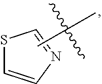
which is optionally substituted with 1 to 3 R^{m} groups independently selected from CH₃, CH₂OH, and C(═O)NH₂;
—C₁ alkyl substituted with a 6-membered optionally substituted heteroaryl;
—C₁ alkyl substituted with 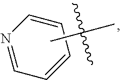
which is optionally substituted with 1 to 3 R^{m} groups independently selected from oxo, CH₃, and CH₂OH;
—C₁ alkyl substituted with 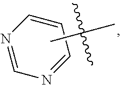
which is optionally substituted with 1 to 3 R^{m} groups independently selected from oxo, CH₃, —C₃ branched alkyl, cyclopropyl, and NHCH₃;
—C₁ alkyl substituted with 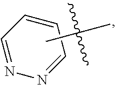
which is optionally substituted with 1 to 3 R^{m} groups independently selected from —OH, oxo, and C₁-C₃ alkyl;
—C₁ alkyl substituted with 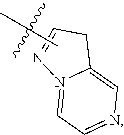
optionally substituted with 1 to 2 R^{m} groups independently selected from oxo and CH₃;
—C₁ alkyl substituted with 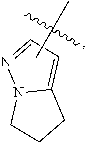
optionally substituted with 1 to 2 R^{m} groups independently selected from oxo and CH₃:
—C₁ alkyl substituted with 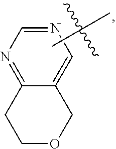
optionally substituted with 1 to 2 R^{m} groups;
—C₁-C₃ alkoxy;
—C₂ alkyl;
—C₂ alkyl substituted with —OH and —C₃-C₆ carbocyclyl (optionally substituted with —OH);
—C₂ alkyl substituted with C₁ alkoxy;
—C₂ alkyl substituted with —OH;
—C₂ alkyl substituted with —NR^{h}Rⁱ, wherein R^{h} is hydrogen, and R is a 6-membered heteroaryl optionally substituted with an R^{m} group selected from —C₁-C₃ alkyl;
—C₂ alkyl substituted with ═NOR^{k}, wherein R^{k} is —C₂ alkyl;
—C₂ alkyl substituted with —NR^{h}S(═O)ₚR^{k}, wherein R^{h} is hydrogen, p is 2 and R^{k} is cyclopropyl;
—C₂ alkyl substituted with —NR^{h}C(═O)NRⁱR^{j}, wherein R^{h} and Rⁱ are hydrogen, and Rⁱ is —C₂ alkyl;
—C₂ alkyl substituted with S(═O)ₚR^{k} wherein p is 2 and R^{k} is CH₃;
—C₂ alkyl substituted with —S(═O)ₚNR^{h}Rⁱ, wherein p is 2 and R^{h} and Rⁱ both hydrogen;
—C₂ alkyl substituted with —S(═O)ₚNR^{h}Rⁱ, wherein R^{h} is hydrogen and Rⁱ is CH₃;
—C₂ alkyl substituted with —OH and 6 membered heterocycle (optionally further substituted with —OH);
—C₂ alkyl substituted with a 6-membered heterocycle, 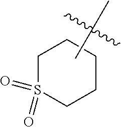
which is further substituted with R^{m}, wherein R¹ is —OR^{k}, wherein R^{k} is —OH;
—C₂ alkyl substituted with —OH and optionally substituted 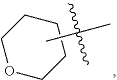
which is further substituted with OH;
—C₂ alkyl substituted with a 5-membered, optionally-substituted heteroaryl;
—C₂ alkyl substituted with 1 to 2 halogen groups and 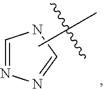
which is optionally substituted with C₁-C₃ alkyl;
—C₂ alkyl substituted with a 6-membered, optionally substituted heteroaryl;
—C₂ alkyl substituted with 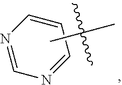
which is optionally substituted with oxo;
—C₃ alkyl optionally substituted with 1-2 —OH groups;
—C₃ haloalkyl substituted with —OH;
—C₃ carbocycle;
—C₃ alkyl substituted with 5-membered, optionally-substitute heteroaryl;
—C₃ alkyl substituted with 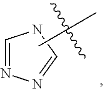
which is optionally substituted with oxo;
—C₃ alkyl substituted with 6-membered heteroaryl, optionally substituted with 1 to 2 oxo groups;
—C₃ alkyl substituted with 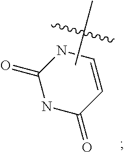
—C₄ alkyl substituted with two —OH groups;
—C₄ branched alkyl substituted with —OH;
—C₄ branched alkyl substituted with —NR^{h}S(═O)ₚR^{k}, wherein R^{h} is hydrogen, p is 2 and R^{k} is CH₃;
—C₄ branched alkoxy substituted with —NR^{h}Rⁱ, wherein wherein R^{h} and Rⁱ both hydrogen;
—C₅ branched alkyl substituted with two —OH groups;
—C₅ branched alkyl substituted with —S(═O)ₚNR^{h}Rⁱ, wherein p is 2 and R^{h} and Rⁱ both hydrogen;
—C₆ aryl, optionally substituted with CF₃;
—C(═O)OR^{k}, wherein R^{k} is CH₃;
—C(═O)NR^{h}Rⁱ, wherein wherein R^{h} is hydrogen and Rⁱ is CH₃;
—C(═O)NR^{h}Rⁱ, wherein wherein R^{h} and Rⁱ are both hydrogen;
—NR^{h}Rⁱ, wherein wherein R^{h} and Rⁱ both hydrogen;
—NR^{h}Rⁱ, wherein R^{h} is hydrogen and Rⁱ is CH₃;
—NR^{h}Rⁱ, wherein wherein R^{h} is hydrogen and Rⁱ is branched —C₄ alkyl substituted with —OH;
—NR^{h}C(═O)R^{k}, wherein R^{h} is hydrogen and R^{k} is CH₃;
—NR^{h}C(═O)R^{k}, wherein wherein R^{h} is hydrogen and R^{k} is a 5 membered heteroaryl, 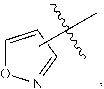
substituted with a group chosen from Cl and cyclopropyl;
—NR^{h}C(═O)R^{k}, wherein R^{h} is hydrogen and R^{k} is unsubstituted 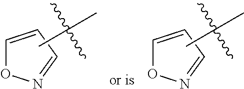
substituted with CH₃;
—NR^{h}S(═O)ₚR^{k}, wherein R^{h} is hydrogen, p is 2, and R^{k} is —C₂ alkyl;
—NR^{h}S(═O)ₚR^{k}, wherein R^{h} is hydrogen, p is 2, and R^{k} is cyclopropyl;
—NR^{h}S(═O)ₚR^{k}, wherein R^{h} is hydrogen, p is 2, and R^{k} is -cyano;
—S(═O)ₚR^{k}, wherein R^{k} is CH₃;
—S(═O)ₚNR^{h}Rⁱ, wherein p is 2, and R^{h} and Rⁱ, are both hydrogen;
4-membered heterocycle, 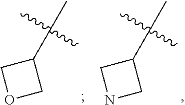 [SMILES: *C1COC1 ; *C1CNC1]
optionally substituted with S(O)₂CH₃;
5-membered heterocycle; 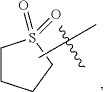
optionally substituted with CH₃; 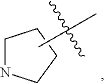
optionally substituted with oxo and CH₃;
6 membered heterocycle;
optionally substituted 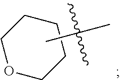 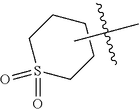
optionally substituted with —OH;
5-membered heteroaryl;
6 membered heteroaryl; 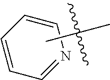
optionally substituted with —C₁-C₃ alkyl; and 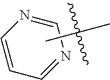
optionally substituted with —C₁-C₃ alkyl.

57. A pharmaceutical composition comprising a compound according to any one of claims 1 to 56.

58. A method of treating a disease mediated by ApoL1, comprising administering a compound according to any one of claims 1 to 56 or a pharmaceutical composition according to claim 57.

59. The method of treating focal segmental glomerulosclerosis (FSGS),comprising administering a compound according to any one of claims 1 to 56 or a pharmaceutical composition according to claim 57.

60. The method of treating non-diabetic kidney disease (NDKD), comprising administering a compound according to any one of claims 1 to 56 or a pharmaceutical composition according to claim 57.

61. The method of treating cancer mediated by ApoL1, comprising administering a compound according to any one of claims 1 to 56 or a pharmaceutical composition according to claim 57.

62. The method of treating cancer according to claim 61, wherein the cancer is pancreatic cancer.

63. The method of treating according to any one of claims 58 to 62, wherein the patient to be treated possesses an APOL1 genetic variants

64. The method of treating according to claim 63, wherein the genetic variant is chosen from G1: S342G:I384M and G2: N388del:Y389del.

65. A method of inhibiting APOL1 activity comprising contacting said APOL1 with at least one compound, tautomer, deuterated derivative, or pharmaceutically acceptable salt according to any one of claims 1 to 56, or a pharmaceutical composition according to claim 57.

66. Use of a compound according to any one of claims 1 to 56 in the manufacture of a medicament for the treatment of an ApoL1 mediated disease.

67. Use of a compound according to any one of claims 1 to 56 in the manufacture of a medicament for the treatment of FSGS.

68. Use of a compound according to any one of claims 1 to 56 in the manufacture of a medicament for the treatment of NDKD.

69. Use of a compound according to any one of claims 1 to 56 in the manufacture of a medicament for the treatment of cancer mediated by ApoL1.

70. Use of a compound according to any one of claims 1 to 56 in the manufacture of a medicament for the treatment of pancreatic cancer mediated by ApoL1.

71. Use of a compound according to any one of claims 1 to 56 in the manufacture of a medicament for inhibiting the activity of ApoL1 in a patient in need thereof.

72. A compound according to any one of claims 1 to 56, or a pharmaceutical composition according to claim 57, for use in inhibiting the activity of ApoL1 in a patient in need thereof.

73. A compound according to any one of claims 1 to 56, or a pharmaceutical composition according to claim 57, for use in treating an ApoL1 mediated disorder.

74. A compound according to any one of claims 1 to 56, or a pharmaceutical composition according to claim 57, for use in treating FSGS.

75. A compound according to any one of claims 1 to 56, or a pharmaceutical composition according to claim 57, for use in treating NDKD.

76. A compound according to any one of claims 1 to 56, or a pharmaceutical composition according to claim 57, for use in treating cancer mediated by ApoL1.

77. A compound according to any one of claims 1 to 56, or a pharmaceutical composition according to claim 57, for use in treating pancreatic cancer mediated by ApoL1.
